(19) United States
(12) Patent Application Publication
(10) Pub. No.: US 2024/0130230 A1
(43) Pub. Date: Apr. 18, 2024

(54) PLURALITY OF HOST MATERIALS, ORGANIC ELECTROLUMINESCENT COMPOUND, AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: Doo-Hyeon MOON, Gyeonggi-do (KR); DaiKyu KIM, Gyeonggi-do (KR); Hyo-Jung LEE, Gyeonggi-do (KR); Yea-Mi SONG, Gyeonggi-do (KR); Kyoung-Jin PARK, Gyeonggi-do (KR); Hyun-Ju KANG, Gyeonggi-do (KR); Ga-Won LEE, Gyeonggi-do (KR); Mi-Ja LEE, Gyeonggi-do (KR); Kyung-Hoon CHOI, Gyeonggi-do (KR)

(21) Appl. No.: 18/446,086

(22) Filed: Aug. 8, 2023

(30) Foreign Application Priority Data
Aug. 25, 2022 (KR) 10-2022-0106869
Jul. 31, 2023 (KR) 10-2023-0099502

(51) Int. Cl. H10K 85/60; C07D 403/04; C07D 403/10; C07D 405/14; C09K 11/06

(52) U.S. Cl. CPC H10K 85/6572; C07D 403/04; C07D 403/10; C07D 405/14; C09K 11/06; H10K 85/654; H10K 85/6574; H10K 50/11

(57) ABSTRACT

The present disclosure relates to a plurality of host materials, organic electroluminescent compounds, and organic electroluminescent devices comprising the same. By comprising specific combinations of compounds according to the present disclosure as the plurality of host materials or by comprising a compound according to the present disclosure, it is possible to provide organic electroluminescent devices having improved lifespan characteristics compared to conventional organic electroluminescent devices.

### TECHNICAL FIELD

The present disclosure relates to a plurality of host materials, an organic electroluminescent compound, and an organic electroluminescent device comprising the same.

### BACKGROUND ART

Since Tang et al. of Eastman Kodak first developed a TPD/Alq₃ bilayer small molecule green organic field emitting device (OLED) consisting of a light emitting layer and a charge transfer layer in 1987, research on organic field emitting devices has rapidly progressed to commercialization. Currently, organic electroluminescent devices mainly use phosphor materials with high luminous efficiency in panel implementation. In many applications, such as TV and lighting, OLEDs face the problem of insufficient lifespan, and high efficiency is still required. In general, the higher the brightness of the OLED, the shorter the lifespan of the OLED. Therefore, long-life OLEDs are required for longer use and higher resolution of the display.

On the other hand, Korean Public Patent Publication Nos. 2019-0113663 and 2015-0036736 disclose carbazole derivative compounds, but do not specifically disclose the specific compounds and specific combinations of host materials claimed herein. Furthermore, there is an ongoing need to develop light emitting materials with improved performance, such as improved lifespan characteristics, compared to previously disclosed organic field emitting devices.

### DISCLOSURE OF THE INVENTION

### Technical Problem

It is an object of the present disclosure to provide a plurality of host materials capable of providing organic electroluminescent devices with improved lifespan characteristics. Another object of the present disclosure is to provide organic electroluminescent compounds with novel structures suitable for application in organic electroluminescent devices. Another object of the present disclosure is to provide an organic electroluminescent device having significantly improved lifespan characteristics by including a compound of the present disclosure or a particular combination of compounds.

### Solution to Problems

As a result of intensive studies to solve the technical problem above, the present inventors found that a plurality of host materials comprising at least one first host compound represented by the following Formula 1 and at least one second host compound represented by the following Formula 2, wherein at least one of the first host compound and the second host compound comprises deuterium; a plurality of host materials comprising at least one first host compound represented by Formula 12 and at least one second host compound represented by Formula 13, wherein the first host compound and the second host compound do not contain deuterium; or a compound represented by Formula 11, so that the present invention was completed.

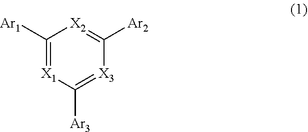

Wherein,
X₁ to X₃ each independently represent N or CRₐ; provided that two of X₁ to X₃ represent N;
Rₐ represents hydrogen, deuterium, or a carbazole group represented by the following Formula 1-1;
Ar₁ to Ar₃ each independently represent hydrogen, deuterium, a substituted or unsubstituted (C6-C30)aryl, or a carbazole group represented by the following Formula 1-1;
Provided that at least one of Rₐ and Ar₁ to Ar₃ represent(s) a carbazole group represented by the following Formula 1-1;

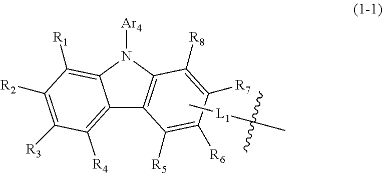

Wherein,
L₁ represents a single bond, or a substituted or unsubstituted (C6-C30)arylene;
one of R₅ to R₈ is connected to L₁, and R₁ to R₄, and R₅ to R₈ not connected to L₁ each independently represent hydrogen, deuterium, a substituted or unsubstituted (C1-C30)alkyl, a substituted or unsubstituted (C6-C30)aryl, or a substituted or unsubstituted (3-to 30-membered)heteroaryl; and
Ar₄ represents a substituted or unsubstituted (C6-C30)aryl, or a substituted or unsubstituted (3- to 30-membered)heteroaryl;

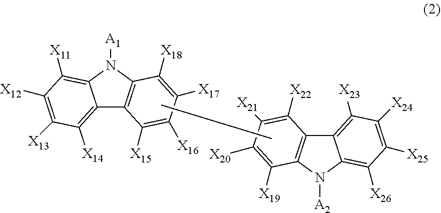

Wherein,
A₁ and A₂ each independently represent a substituted or unsubstituted (C6-C30)aryl, a substituted or unsubstituted dibenzofuranyl, a substituted or unsubstituted dibenzothiophenyl, or a substituted or unsubstituted carbazoly;
any one of X₁₅ to X₁₈ and any one of X₁₉ to X₂₂ are connected to each other to form a single bond; and
X₁₁ to X₁₄, X₂₃ to X₂₆, and X₁₅ to X₂₂, which do not form a single bond, each independently represent hydrogen, deuterium, a substituted or unsubstituted (C6-C30)aryl, or a substituted or unsubstituted (3- to 30-membered)heteroaryl; or may be linked to an adjacent substituent to form a ring(s).

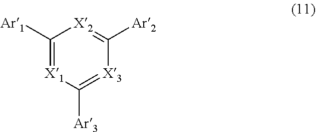

Wherein,
X′₁ to X′₃ each independently represent N or CR′ₐ; provided that at least two of X′₁ to X′₃ represent N;
R′ₐ represents hydrogen or deuterium;
Ar′₁ to Ar′₃ each independently represent (C6-C30)aryl unsubstituted or substituted with at least one of deuterium and (C6-C30)aryl, or are represented by the following Formula A; provided that at least one of Ar′₁ to Ar′₃ is(are) represented by the following Formula A;

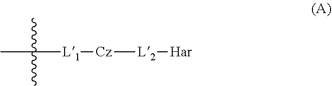

Wherein,
L′₁ represents a single bond, or a substituted or unsubstituted (C6-C12)arylene;
Cz represents a substituted or unsubstituted carbazolylene;
L′₂ represents a substituted or unsubstituted (C6-C12)arylene; and
HAr represents a substituted or unsubstituted dibenzofuranyl, a substituted or unsubstituted carbazolyl, or a substituted or unsubstituted dibenzothiophenyl.

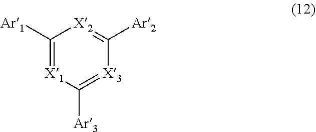

Wherein,
X′₁ to X′₃ each independently represent N or CH; provided that at least two of X₁ to X′₃ represent N;
Ar′₁ to Ar′₃ each independently represent (C6-C30)aryl unsubstituted or substituted with (C6-C30)aryl, or are represented by the following Formula A; provided that at least one of Ar′₁ to Ar′₃ is(are) represented by the following Formula A;

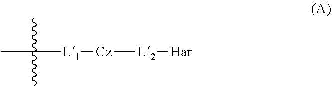

Wherein,
L′₁ represents a single bond, or a substituted or unsubstituted (C6-C12)arylene;
Cz represents a substituted or unsubstituted carbazolyene;
L′₂ represents a substituted or unsubstituted (C6-C12)arylene; and
HAr represents a substituted or unsubstituted dibenzofuranyl, a substituted or unsubstituted carbazolyl, or a substituted or unsubstituted dibenzothiophenyl.

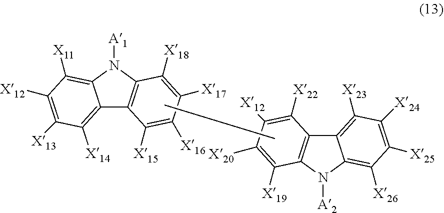

Wherein,
A′₁ and A′₂ each independently represent a substituted or unsubstituted (C6-C30)aryl, a substituted or unsubstituted dibenzofuranyl, a substituted or unsubstituted dibenzothiophenyl, or a substituted or unsubstituted carbazoly;
any one of X′₁₅ to X′₁₈ and any one of X′₁₉ to X′₂₂ are connected to each other to form a single bond; and
X′₁₁ to X′₁₄, X′₂₃ to X′₂₆, and X′₁₅ to X′₂₂, which do not form a single bond, each independently represent hydrogen, a substituted or unsubstituted (C6-C30)aryl, or a substituted or unsubstituted (3- to 30-membered)heteroaryl; or may be linked to an adjacent substituent to form a ring(s).

### Advantageous Effects of Invention

By comprising specific combinations of compounds according to the present disclosure as a plurality of host materials, or by comprising a compound according to the present disclosure, an organic electroluminescent device can be prepared that exhibit significantly improved lifespan characteristics compared to conventional organic electroluminescent devices.

### EMBODIMENTS OF THE INVENTION

Hereinafter, the present disclosure will be described in detail. However, the following description is intended to explain the invention, and is not meant in any way to restrict the scope of the invention.

The present disclosure relates to a plurality of host materials including a first host compound comprising at least one compound represented by Formula 1 and a second host compound comprising at least one compound represented by Formula 2, wherein at least one of the first host compound and the second host compound comprises deuterium, and an organic electroluminescent device comprising said host materials. According to one embodiment of the present disclosure, the first host compound does not contain deuterium, and the second host compound may contain deuterium. According to another embodiment of the present disclosure, both the first host compound and the second host compound may comprise deuterium.

The present disclosure relates to an organic electroluminescent compound represented by Formula 11, an organic electroluminescent material comprising the same, and an organic electroluminescent device.

The present disclosure relates to a plurality of host materials including a first host compound comprising at least one compound represented by Formula 12 and a second host compound comprising at least one compound represented by Formula 13, and an organic electroluminescent device comprising said host materials. According to one embodiment of the present disclosure, the first host compound comprising a compound represented by Formula 12 and the second host compound comprising a compound represented by Formula 13 do not contain deuterium.

Herein, “organic electroluminescent compound” means a compound that may be used in an organic electroluminescent device, and may be comprised in any material layer constituting an organic electroluminescent device, as necessary.

Herein, “organic electroluminescent material” means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transfer material, a hole auxiliary material, a light emitting auxiliary material, an electron blocking material, a light emitting material (including a host material and a dopant material), an electron buffer material, a hole blocking material, an electron transfer material, an electron injection material, etc.

Herein, “a plurality of host materials” means an organic electroluminescent material comprising a combination of at least two host materials. It may mean both a material before being comprised in an organic electroluminescent device (e.g., before vapor deposition) and a material after being comprised in an organic electroluminescent device (e.g., after vapor deposition). A plurality of host materials of the present disclosure may be comprised in any light-emitting layer constituting an organic electroluminescent device. The at least two compounds comprised in a plurality of host materials may be comprised together in one light-emitting layer, or may each be comprised in separate light-emitting layers. When at least two compounds are comprised in one light-emitting layer, the at least two compounds may be mixture-evaporated to form a layer or may be individually and simultaneously co-evaporated to form a layer.

Herein, “(C1-C30)alkyl” means a straight-chain or branched-chain alkyl having from 1 to 30 carbons comprising the chain, preferably from 1 to 20 carbons, and more preferably from 1 to 10 carbons. Specific examples of said alkyl include methyl, ethyl, n-propyl, isopropyl, n-butyl, isobutyl, tert-butyl, sec-butyl, etc. Herein, “(C3-C30)cycloalkyl” means a monocyclic or polycyclic hydrocarbon having from 3 to 30 carbons in the ring skeleton, preferably from 3 to 20 carbons, more preferably from 3 to 7 carbons. Examples of said cycloalkyl include cyclopropyl, cyclobutyl, cyclopentyl, cyclohexyl, cyclopentylmethyl, cyclohexylmethyl, etc. Herein, the term “(3-7 membered) heterocycloalkyl” means a cycloalkyl having a ring skeleton number of 3 to 7 atoms and containing at least one heteroatom selected from the group consisting of B, N, O, S, Si, and P, preferably at least one heteroatom selected from O, S, and N. Examples include tetrahydrofuran, pyrrolidine, thiolane, and tetrahydropyran.

Herein, “(C6-C30)aryl(ren)” means a monocyclic or fused cyclic radical derived from an aromatic hydrocarbon having a ring skeleton of 6 to 30 carbons, which may be partially saturated. The aryl includes those having a spiro structure. Examples of said aryl include phenyl, biphenyl, terphenyl, naphthyl, non-naphthyl, phenylnaphthyl, naphthylphenyl, fluorenyl, phenylfluorenyl, diphenylfluorenyl, benzofluorenyl, dibenzofluorenyl, phenanthrenyl, phenylphenanthrenyl, anthracenyl, indenyl, triphenylenyl, pyrenyl, tetracenyl, perylenyl, chrysenyl, naphthacenyl, fluoranthenyl, spirobifluorenyl, spiro[fluorene-benzofluorene]yl, azulenyl, tetramethyldihydrophenanthrenyl, etc. Specifically, examples of aryl include phenyl, 1-naphthyl, 2-naphthyl, 1-anthryl, 2-anthryl, 9-anthryl, benzanthryl, 1-phenanthryl, 2-phenanthryl, 3-phenanthryl, 4-phenanthryl, 9-phenanthryl, naphthacenyl, pyrenyl, 1-chrysenyl, 2-chrysenyl, 3-chrysene, 4-chrysene, 5-chrysene, 6-chrysene, benzo[c]phenanthryl, benzo[g]chrysene, 1-triphenylenyl, 2-triphenylenyl, 3-triphenylenyl, 4-triphenylenyl, 1-fluorenyl, 2-fluorenyl, 3-fluorenyl, 4-fluorenyl, 9-fluorenyl, benzo[a]fluorenyl, benzo[b]fluorenyl, benzo[c]fluorenyl, dibenzofluorenyl, 2-biphenylyl, 3-biphenylyl, 4-biphenylyl, o-terphenyl, m-terphenyl-4-yl, m-terphenyl-3-yl, m-terphenyl-2-yl, p-terphenyl-4-yl, p-terphenyl-3-yl, p-tert-terphenyl-2-yl, m-quaterphenyl, 3-fluoranthenyl, 4-fluoranthenyl, 8-fluoranthenyl, 9-fluoranthenyl, benzofluoranthenyl, o-tolyl, m-tolyl, p-tolyl, 2,3-xylyl, 3,4-xylyl, 2,5-xylyl, mesityl, o-cumenyl, m-cumenyl, p-cumenyl, p-tert-Butylphenyl, p-(2-phenylpropyl)phenyl, 4′-methylbiphenyl, 4″-tert-Butyl-p-terphenyl-4-yl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 11,11-Dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-Dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-Dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-Dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-Dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-Diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-Diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-Diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-Diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc.

Herein, “(3- to 30-membered) heteroaryl” means an aryl group having a ring skeleton number of 3 to 30 atoms and containing one or more heteroatoms selected from the group consisting of B, N, O, S, Si, and P. The number of heteroatoms is preferably 1 to 4, either monocyclic or fused cyclic, and may be partially saturated. The number of heteroatoms is preferably from 1 to 4, and may be a single ring system or a fused ring system condensed with one or more benzene rings, and may be partially saturated. As used herein, heteroaryl also includes forms in which one or more heteroaryl or aryl groups are linked to a heteroaryl group by a single bond, and also includes those having a spiro structure. Examples of heteroaryls include furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidine, pyridazinyl, and other monocyclic heteroaryls, benzofuranyl, benzothiophenyl, and isobenzofuranyl, dibenzofuranyl, dibenzothiophenyl, dibenzoselenophenyl, naphthobenzofuranyl, naphthobenzothiophenyl, benzofuroquinolinyl, benzofuroquinazolinyl, benzofuronaphthyridinyl, benzofuropyrimidinyl, naphthofuropyrimidinyl, benzothienoquinolinyl, benzothienoquinazolinyl, benzothienonaphthyridinyl, benzothienopyrimidinyl, naphthothienopyrimidinyl, pyrimidoindolyl, benzopyrimidoindolyl, benzofurfurylpyrazinyl, naphthofurfurylpyrazinyl, benzothienopyrazinyl, naphthothienopyrazinyl, pyrazinodiol, benzopyrazinodiol, benzoimidazolyl, benzothiazolyl, benzoisothiazolyl, benzoisoxazolyl, benzooxazolyl, isoindolyl, indolyl, indazolyl, benzothiadiazolyl, quinolyl, isoquinolyl, cinnolyl, quinazolinyl, quinoxalinyl, carbazolyl, benzocarbazolyl, dibenzocarbazolyl, phenoxazinyl, phenanthridinyl, benzodioxolyl, dihydroacridinyl, benzotriazolyl, phenazineyl, imidazopyridyl, chromenoquinazolinyl, thiocromenoquinazolinyl, dimethylbenzopyrimidinyl, indolocarbazolyl, indenocarbazolyl, and fused cyclic heteroaryls. More specifically, examples of the above heteroaryls include 1-pyrrolyl, 2-pyrrolyl, 3-pyrrolyl, pyrazinyl, 2-pyridinyl, 2-pyrimidinyl, 4-pyrimidinyl, 5-pyrimidinyl, 6-pyrimidinyl, 1,2,3-triazine-4-yl, 1,2,4-triazine-3-yl, 1,3,5-triazine-2-yl, 1-imidazolyl, 2-imidazolyl, 1-pyrazolyl, 1-indolidinyl, 2-indolidinyl, 3-indolidinyl, 5-indolidinyl, 6-indolidinyl, 7-indolidinyl, 8-indolidinyl, 2-imidazopyridinyl, 3-imidazopyridinyl, 5-imidazopyridinyl, 6-imidazopyridinyl, 7-imidazopyridinyl, 8-imidazopyridinyl, 3-pyridinyl, 4-pyridinyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-Isobenzofuranyl, 7-Isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-Carbazolyl, 9-Carbazolyl, Azacarbazolyl-1-yl, Azacarbazolyl-2-yl, Azacarbazolyl-3-yl, Azacarbazolyl-4-yl, Azacarbazolyl-5-yl, Azacarbazolyl-6-yl, Azacarbazolyl-7-yl, Azacarbazolyl-8-yl, Azacarbazolyl-9-yl, 1-Phenanthridinyl, 2-Phenanthridinyl, 3-Phenanthridinyl, 4-Phenanthridinyl, 6-Phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrole-5-yl, 3-methylpyrrole-1-yl, 3-methylpyrrole-2-yl, 3-methylpyrrole-4-yl, 3-methylpyrrole-5-yl, 2-tert-butylpyrrole-4-yl, 3-(2-phenylpropyl)pyrrole-1-yl, 2-methyl-1-indolyl, 4-methyl-1-indolyl, 2-methyl-3-indolyl, 4-methyl-3-indolyl, 2-tert-butyl-1-indolyl, 4-tert-butyl-1-indolyl, 2-tert-butyl-3-indolyl, 4-tert-butyl-3-indolyl, 1-Dibenzofuranyl, 2-Dibenzofuranyl, 3-Dibenzofuranyl, 4-Dibenzofuranyl, 1-Dibenzothiophenyl, 2-Dibenzothiophenyl, 3-Dibenzothiophenyl, 4-Dibenzothiophenyl, 1-Naphtho-[1,2-b]-benzofuranyl, 2-Naphtho-[1,2-b]-benzofuranyl, 3-Naphtho-[1,2-b]-benzofuranyl, 4-Naphtho-[1,2-b]-benzofuranyl, 5-Naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-Naphtho-[2,1-b]-benzofuranyl, 2-Naphtho-[2,1-b]-benzofuranyl, 3-Naphtho-[2,1-b]-benzofuranyl, 4-Naphtho-[2,1-b]-benzofuranyl, 5-Naphtho-[2,1-b]-benzofuranyl, 6-Naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-Naphtho-[2,1-b]-benzothiophenyl, 2-Benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzocelenophenyl, 2-dibenzocelenophenyl, 3-dibenzocelenophenyl, 4-dibenzocelenophenyl, etc. Herein, “halogen” includes F, Cl, Br, and I atoms.

In addition, “ortho; o-”, “meta; m-”, and “para; p-” are prefixes that refer to the relative positions of substituents. Ortho indicates that two substituents are adjacent to each other; for example, in a benzene substituent, when the substituents are at positions 1 and 2, they are said to be in ortho position. Meta indicates that two substituents are at positions 1 and 3, for example, in a benzene substituent when the substituents are at positions 1 and 3, it is called the meta position. Para indicates that two substituents are at positions 1 and 4, for example, in a benzene substituent the substituents are at positions 1 and 4, which is called the para position.

Herein, the term “substituted or unsubstituted” refers to the replacement of a hydrogen atom in a functional group with another atom or another functional group (i.e., a substituent), including the replacement of a substituent with a group having two or more substituents. For example, a “substituent having two or more substituents connected” may be a pyridine-triazine, i.e., a pyridine-triazine may be interpreted as having one heteroaryl substituent, or it may be interpreted as having two heteroaryl substituents connected. In the formulas herein, substituted alkyl, substituted aryl, substituted arylene, substituted heteroaryl, substituted dibenzofuranyl, substituted dibenzothiophenyl, substituted carbazolyl, substituted carbazolyl, and substituted carbazolylene may each be independently selected from deuterium, halogen, cyano, carboxyl, nitro, hydroxy, (C1-C30)alkyl, halo(C1-C30)alkyl, (C2-C30)alkenyl, (C2-C30)alkynyl, (C1-C30)alkoxy, (C1-C30)alkylthio, (C3-C30)cycloalkyl, (C3-C30)cycloalkenyl, (3-7 membered)heterocycloalkyl, (C6-C30)aryloxy, (C6-C30)arylthio, (3- to 30-membered)heteroaryl unsubstituted or substituted with at least one of deuterium and (C6-C30)aryl, (C6-C30)aryl unsubstituted or substituted with deuterium and at least one of (3- to 30-membered) heteroaryl, tri(C1-C30)alkylsilyl, tri(C6-C30)arylsilyl, di(C1-C30)alkyl(C6-C30)arylsilyl, (C1-C30)alkyldi(C6-C30)arylsilyl, fused ring of aliphatic ring at (C3-C30) and aromatic ring at (C6-C30), amino, mono- or di-(C1-C30)alkylamino, mono- or di-(C2-C30)alkenylamino, (C1-C30)alkyl(C2-C30)alkenylamino, Substituted or unsubstituted mono- or di-(C6-C30)arylamino, (C1-C30)alkyl(C6-C30)arylamino, mono- or di-(3- to 30-membered)heteroarylamino, (C1-C30)alkyl(3- to 30-membered)heteroarylamino, (C2-C30)alkenyl(C6-C30)arylamino, (C2-C30)alkenyl (3- to 30-membered) heteroaryl amino, (C6-C30)aryl (3- to 30-membered) heteroaryl amino, (C1-C30)alkylcarbonyl, (C1-C30)alkoxycarbonyl, (C6-C30)arylcarbonyl, (C6-C30)arylphosphinyl, di(C6-C30)arylboronyl, di(C1-C30)alkylboronyl, (C1-C30)alkyl(C6-C30)arylboronyl, (C6-C30)ar(C1-C30)alkyl, and (C1-C30)alkyl(C6-C30)aryl, which may be further substituted with deuterium. According to one embodiment of the present disclosure, said substituent is each independently one or more selected from the group consisting of deuterium; (C6-C25)aryl unsubstituted or substituted with deuterium; and (5- to 25-membered)heteroaryl unsubstituted or substituted with deuterium. According to another embodiment of the present disclosure, said substituent is each independently at least one selected from the group consisting of deuterium; (C6-C18)aryl unsubstituted or substituted with deuterium; and (5- to 20-membered)heteroaryl unsubstituted or substituted with deuterium. For example, said substituent may be one or more selected from the group consisting of deuterium, phenyl, naphthyl, triphenylenyl, dibenzofuranyl, and dibenzothiophenyl, which may be further substituted with one or more deuterium.

Herein, the term “a ring formed in linking to an adjacent substituent” means a substituted or unsubstituted (3- to 30-membered) mono- or polycyclic, alicyclic, aromatic ring, or a combination thereof, formed by linking or fusing two or more adjacent substituents, preferably a substituted or unsubstituted (3- to 25-membered) mono- or polycyclic, alicyclic, aromatic ring, or a combination thereof, more preferably (5-25 membered) mono- or polycyclic aromatic ring unsubstituted or substituted with one or more of (C6-C18)aryl and (5- to 25-membered)heteroaryl.

Furthermore, the formed ring may comprise one or more heteroatoms selected from B, N, O, S, Si, and P, preferably one or more heteroatoms selected from N, O, and S. For example, the ring may be a benzene ring, a cyclopentane ring, an indane ring, a fluorene ring unsubstituted or substituted with one or more phenyl(s), a phenanthrene ring, an indole ring, a xanthene ring, etc.

Herein, heteroaryl and heterocycloalkyl may each independently comprise one or more heteroatoms selected from B, N, O, S, Si, and P. Further, said heteroatoms may be combined with one or more selected from the group consisting of hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted mono- or di-(C6-C30)arylamino, a substituted or unsubstituted mono- or di-(3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, and a substituted or unsubstituted (C6-C30)aryl(3- to 30-membered)heteroarylamino.

The compound represented by Formula 1 of the present application will be described in more detail as follows.

In Formula 1 above, X₁ to X₃ each independently represent N or CRₐ; provided that at least two of X₁ to X₃ are N. According to one embodiment of the present disclosure, any two of X₁ to X₃ are N and the other is CRₐ. According to another embodiment of the present disclosure, all of X₁ to X₃ are N.

In Formula 1 above, Rₐ represents hydrogen, deuterium, or a carbazole group represented by Formula 1-1 above.

In Formula 1 above, Ar₁ to Ar₃ each independently represent hydrogen, deuterium, substituted or unsubstituted (C6-C30)aryl, or a carbazole group represented by Formula 1-1 above. According to one embodiment of the present disclosure, Ar₁ and Ar₃ each independently represent hydrogen, deuterium, (C6-C25)aryl unsubstituted or substituted with at least one of deuterium and (3- to 30-membered) heteroaryl, or a carbazole group represented by Formula 1-1 above. Specifically, the (C6-C30)aryl of Ar₁ to Ar₃ each independently represent phenyl, biphenyl, terphenyl, quaterphenyl, naphthyl, phenylnaphthyl, naphthylphenyl, triphenylenyl, phenanthrenyl, or a combination thereof. The substituent of the substituted (C6-C30)aryl of Ar₁ to Ar₃ may be one or more selected from the group consisting of deuterium and (3- to 30-membered) heteroaryl. For example, Ar₁ to Ar₃ may be each independently hydrogen, deuterium, phenyl unsubstituted or substituted with dibenzofuranyl or dibenzothiophenyl, biphenyl, terphenyl, or quaterphenyl, etc. They may be further substituted with deuterium.

In Formula 1 above, at least one of Rₐ and Ar₁ to Ar₃ is(are) a carbazole group represented by Formula 1-1 above. According to one embodiment of the present disclosure, any one of Rₐ and Ar₁ to Ar₃ is a carbazole group represented by Formula 1-1 above.

In Formula 1-1 above, L₁ represents a single bond, or a substituted or unsubstituted (C6-C30)arylene. According to one embodiment of the invention, L₁ represents a single bond, or (C6-C25)arylene unsubstituted or substituted with at least one of deuterium and (C6-C30)aryl. According to another embodiment of the present disclosure, L₁ is a single bond, or (C6-C18)arylene unsubstituted or substituted with at least one of deuterium and (C6-C18)aryl. For example, L₁ may be a single bond, phenylene, or biphenylene unsubstituted or substituted with phenyl, etc. They may be further substituted with deuterium.

In Formula 1-1 above, any one of R₅ to R₈ is connected to L₁, and R₁ to R₄, and R₅ to R₈ not connected to L₁ each independently represent hydrogen, deuterium, a substituted or unsubstituted (C1-C30)alkyl, a substituted or unsubstituted (C6-C30)aryl, or a substituted or unsubstituted (3- to 30-membered)heteroaryl. According to one embodiment of the present disclosure, R₁ to R₃ each independently represent hydrogen, deuterium, (C6-C25)aryl unsubstituted or substituted with deuterium, or (5- to 25-membered)heteroaryl unsubstituted or substituted with deuterium. According to another embodiment of the present disclosure, R₁ to R₈ each independently represent hydrogen, deuterium, (C6-C25)aryl unsubstituted or substituted with deuterium, or (13-membered)heteroaryl unsubstituted or substituted with deuterium. For example, R₁ to R₈ may be each independently hydrogen, deuterium, phenyl, biphenyl, phenanthrenyl, terphenyl, quaterphenyl, naphthyl, phenylnaphthyl, naphthylphenyl, dibenzofuranyl, carbazolyl, dibenzothiophenyl, or a combination thereof. They may be further substituted with deuterium.

In Formula 1-1 above, Ar₄ represents a substituted or unsubstituted (C6-C30)aryl, or a substituted or unsubstituted (3- to 30-membered)heteroaryl. According to one embodiment of the present disclosure, Ar₄ represents (C6-C25)aryl unsubstituted or substituted with at least one of deuterium and (3- to 30-membered)heteroaryl, or (5- to 25-membered)heteroaryl unsubstituted or substituted with deuterium. According to another embodiment of the present disclosure, Ar₄ represents (C6-C18)aryl unsubstituted or substituted with at least one of deuterium and (13-membered)heteroaryl, or (13-membered)heteroaryl unsubstituted or substituted with deuterium. Specifically, Ar₄ may be a substituted or unsubstituted, phenyl, biphenyl, terphenyl, dibenzofuranyl, or dibenzothiophenyl, etc. These substituents may be each independently one selected from the group consisting of deuterium, dibenzofuranyl unsubstituted or substituted with deuterium, dibenzothiophenyl unsubstituted or substituted with deuterium, and carbazolyl unsubstituted or substituted with deuterium. For example, Ar₄ may be phenyl unsubstituted or substituted with dibenzofuranyl or dibenzothiophenyl, biphenyl, terphenyl, dibenzofuranyl, or dibenzothiophenyl, etc. They may be further substituted with deuterium.

According to one embodiment of the present disclosure, the above Formula 1-1 may be represented by the following Formula A-1.

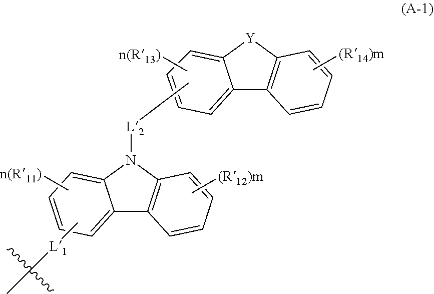

in Formula A-1,
L′₁ represents a single bond or substituted or unsubstituted (C6-C12)arylene;
L′₂ represents a substituted or unsubstituted (C6-C12)arylene;
Y represents —O—, —S—, or —N(R′₁)—;
R′₁ represents a substituted or unsubstituted (C6-C12)aryl, or a substituted or unsubstituted dibenzofuranyl, a substituted or unsubstituted carbazolyl, or a substituted or unsubstituted dibenzothiophenyl;
R′₁₁ to R′₁₄ each independently represent hydrogen, deuterium, a substituted or unsubstituted (C6-C12)aryl, or a substituted or unsubstituted dibenzofuranyl, a substituted or unsubstituted carbazolyl, or a substituted or unsubstituted dibenzothiophenyl;
n is an integer of 1 to 3, m is an integer of 1 to 4;
when m and n are 2 or more, each of R′₁₁ to R′₁₄ may be the same or different from each other.

According to one embodiment of the present disclosure, the deuterium substitution rate in one compound represented by Formula 1 may be from about 30% to about 100%, preferably from about 40% to about 100%, more preferably from about 50% to about 100%, and still more preferably from about 60% to about 100%. According to another embodiment of the present disclosure, Formula 1 may comprise more than one hydrogen, i.e. in one compound represented by Formula 1, the deuterium substitution rate may be from about 30% to less than 100%, preferably from about 40% to less than 100%, more preferably from about 50% to less than 100%, more preferably from about 60% to less than 100%. The upper limit of said substitution rate may be 100%, or may be less than 100%, for example, about 99%. In other words, the compound of Formula 1 may be a compound in which hydrogen is wholly or partially replaced by deuterium. A compound of Formula 1 substituted at said deuterium substitution rate may increase the stability of the compound due to increased bond dissociation energy upon deuteration, and an organic electroluminescent device comprising said compound may exhibit improved lifespan characteristics.

According to one embodiment of the present disclosure, the compound represented by Formula 1 may be one or more selected from the following compounds, but is not limited thereto.

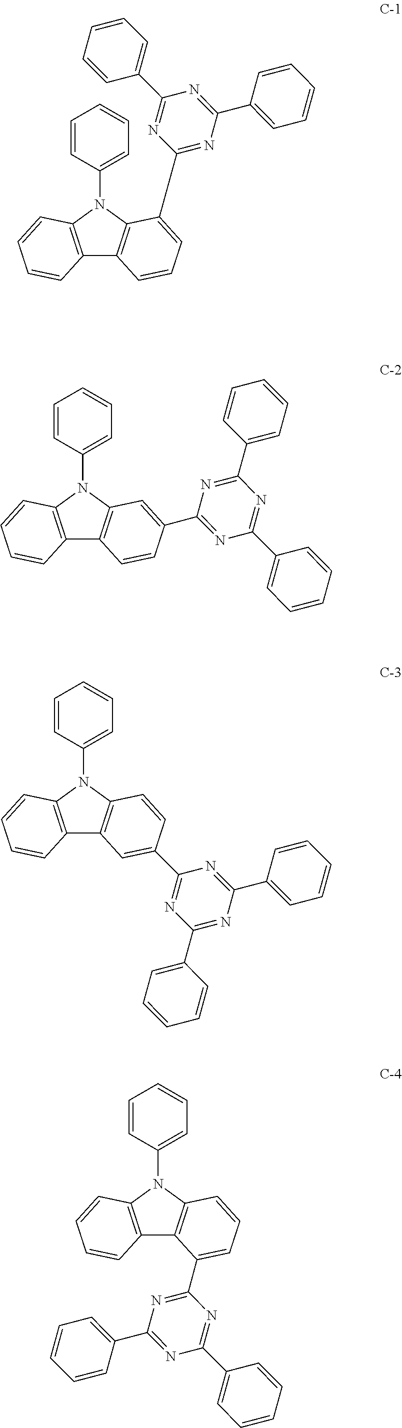 [SMILES: c1ccc(-c2nc(-c3ccccc3)nc(-c3cccc4c5ccccc5n(-c5ccccc5)c34)n2)cc1 ; c1ccc(-c2nc(-c3ccccc3)nc(-c3ccc4c5ccccc5n(-c5ccccc5)c4c3)n2)cc1 ; c1ccc(-c2nc(-c3ccccc3)nc(-c3ccc4c(c3)c3ccccc3n4-c3ccccc3)n2)cc1 ; c1ccc(-c2nc(-c3ccccc3)nc(-c3cccc4c3c3ccccc3n4-c3ccccc3)n2)cc1]

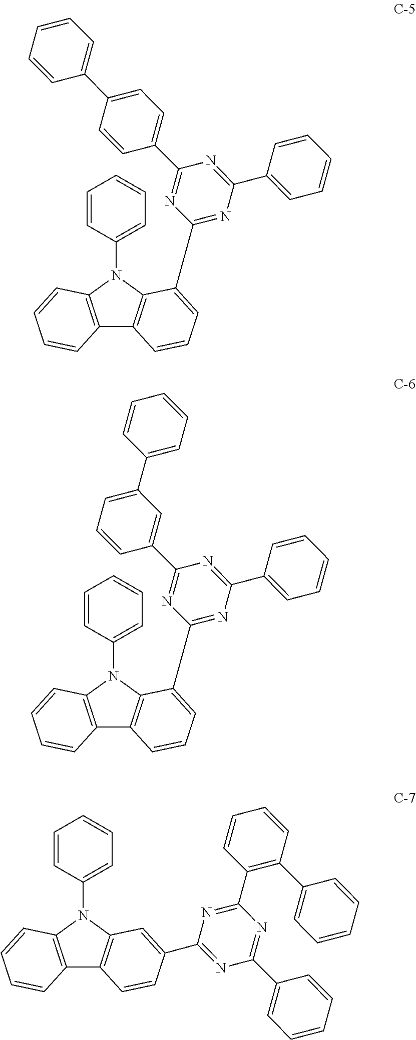

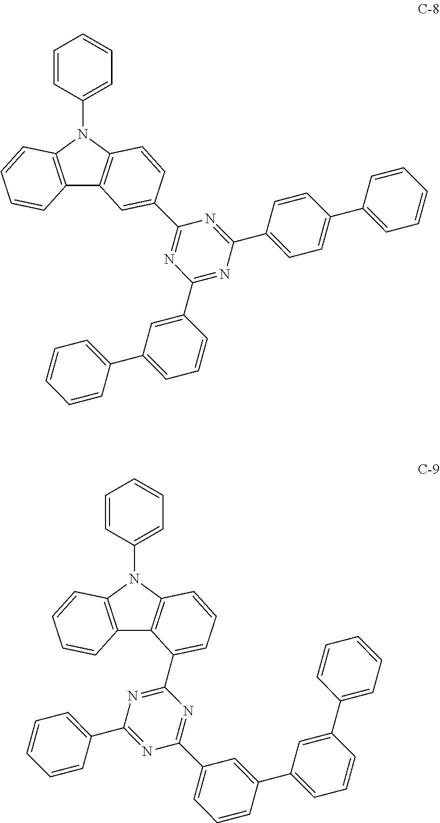 [SMILES: c1ccc(-c2ccc(-c3nc(-c4cccc(-c5ccccc5)c4)nc(-c4ccc5c(c4)c4ccccc4n5-c4ccccc4)n3)cc2)cc1 ; c1ccc(-c2cccc(-c3cccc(-c4nc(-c5ccccc5)nc(-c5cccc6c5c5ccccc5n6-c5ccccc5)n4)c3)c2)cc1]

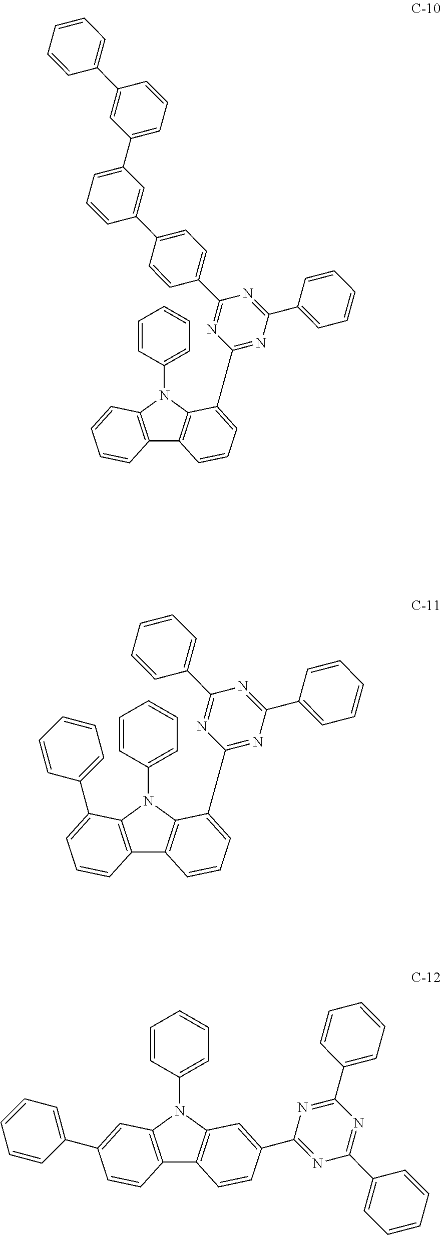 [SMILES: c1ccc(-c2cccc(-c3cccc(-c4ccc(-c5nc(-c6ccccc6)nc(-c6cccc7c8ccccc8n(-c8ccccc8)c67)n5)cc4)c3)c2)cc1 ; c1ccc(-c2nc(-c3ccccc3)nc(-c3cccc4c5cccc(-c6ccccc6)c5n(-c5ccccc5)c34)n2)cc1 ; c1ccc(-c2ccc3c4ccc(-c5nc(-c6ccccc6)nc(-c6ccccc6)n5)cc4n(-c4ccccc4)c3c2)cc1]

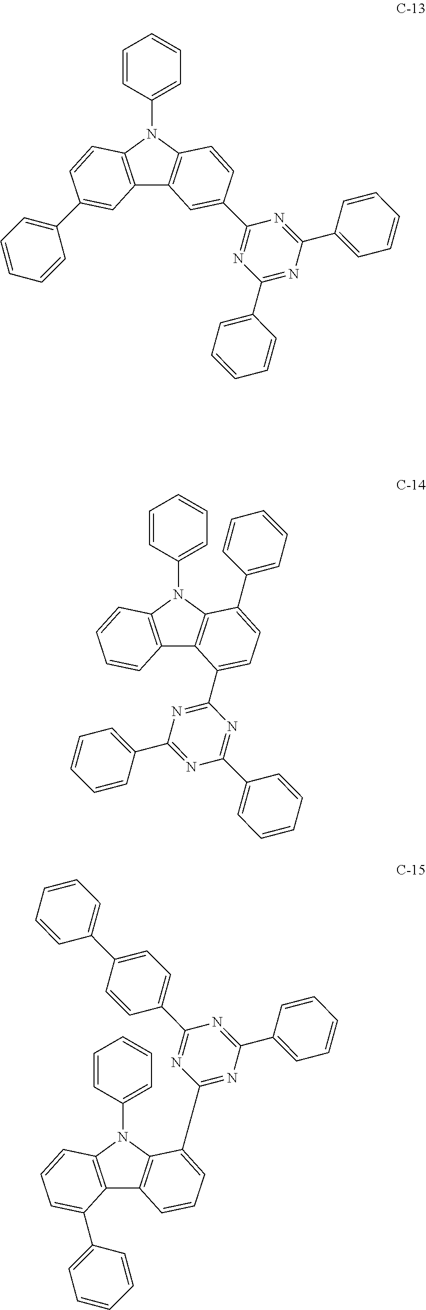 [SMILES: c1ccc(-c2ccc3c(c2)c2cc(-c4nc(-c5ccccc5)nc(-c5ccccc5)n4)ccc2n3-c2ccccc2)cc1 ; c1ccc(-c2nc(-c3ccccc3)nc(-c3ccc(-c4ccccc4)c4c3c3ccccc3n4-c3ccccc3)n2)cc1 ; c1ccc(-c2ccc(-c3nc(-c4ccccc4)nc(-c4cccc5c6c(-c7ccccc7)cccc6n(-c6ccccc6)c45)n3)cc2)cc1]

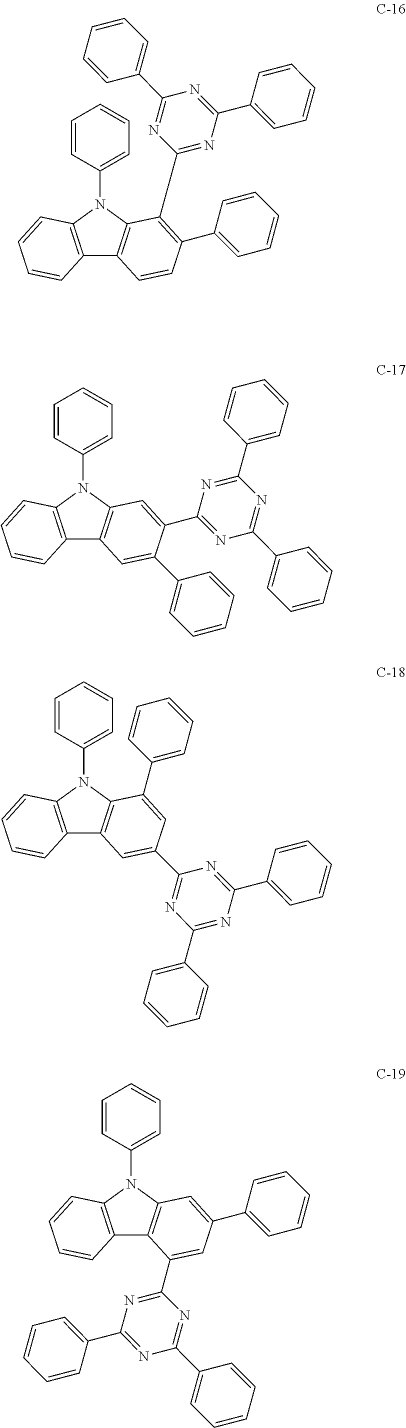 [SMILES: c1ccc(-c2nc(-c3ccccc3)nc(-c3c(-c4ccccc4)ccc4c5ccccc5n(-c5ccccc5)c34)n2)cc1 ; c1ccc(-c2nc(-c3ccccc3)nc(-c3cc4c(cc3-c3ccccc3)c3ccccc3n4-c3ccccc3)n2)cc1 ; c1ccc(-c2nc(-c3ccccc3)nc(-c3cc(-c4ccccc4)c4c(c3)c3ccccc3n4-c3ccccc3)n2)cc1 ; c1ccc(-c2cc(-c3nc(-c4ccccc4)nc(-c4ccccc4)n3)c3c4ccccc4n(-c4ccccc4)c3c2)cc1]

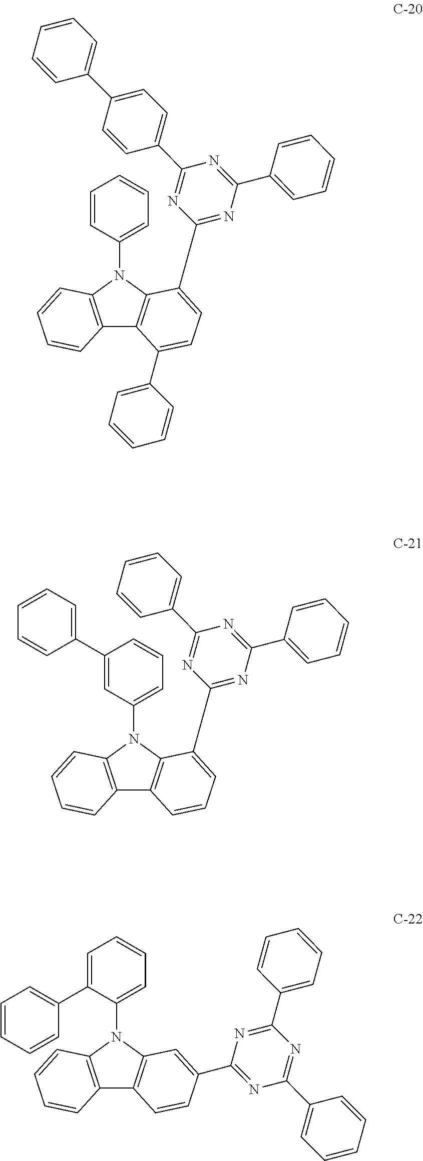 [SMILES: c1ccc(-c2ccc(-c3nc(-c4ccccc4)nc(-c4ccc(-c5ccccc5)c5c6ccccc6n(-c6ccccc6)c45)n3)cc2)cc1 ; c1ccc(-c2cccc(-n3c4ccccc4c4cccc(-c5nc(-c6ccccc6)nc(-c6ccccc6)n5)c43)c2)cc1 ; c1ccc(-c2nc(-c3ccccc3)nc(-c3ccc4c5ccccc5n(-c5ccccc5-c5ccccc5)c4c3)n2)cc1]

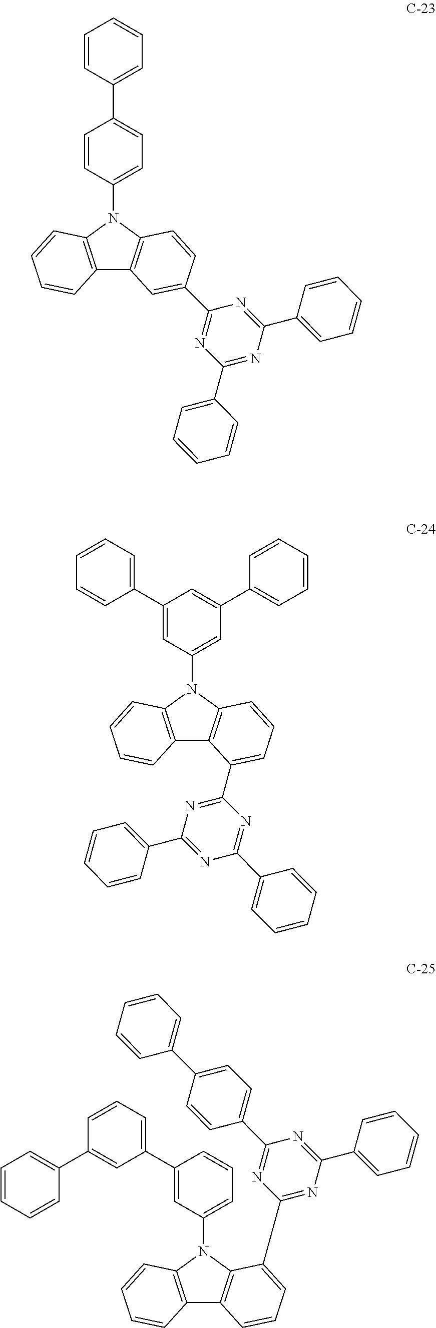 [SMILES: c1ccc(-c2ccc(-n3c4ccccc4c4cc(-c5nc(-c6ccccc6)nc(-c6ccccc6)n5)ccc43)cc2)cc1 ; c1ccc(-c2cc(-c3ccccc3)cc(-n3c4ccccc4c4c(-c5nc(-c6ccccc6)nc(-c6ccccc6)n5)cccc43)c2)cc1 ; c1ccc(-c2ccc(-c3nc(-c4ccccc4)nc(-c4cccc5c6ccccc6n(-c6cccc(-c7cccc(-c8ccccc8)c7)c6)c45)n3)cc2)cc1]

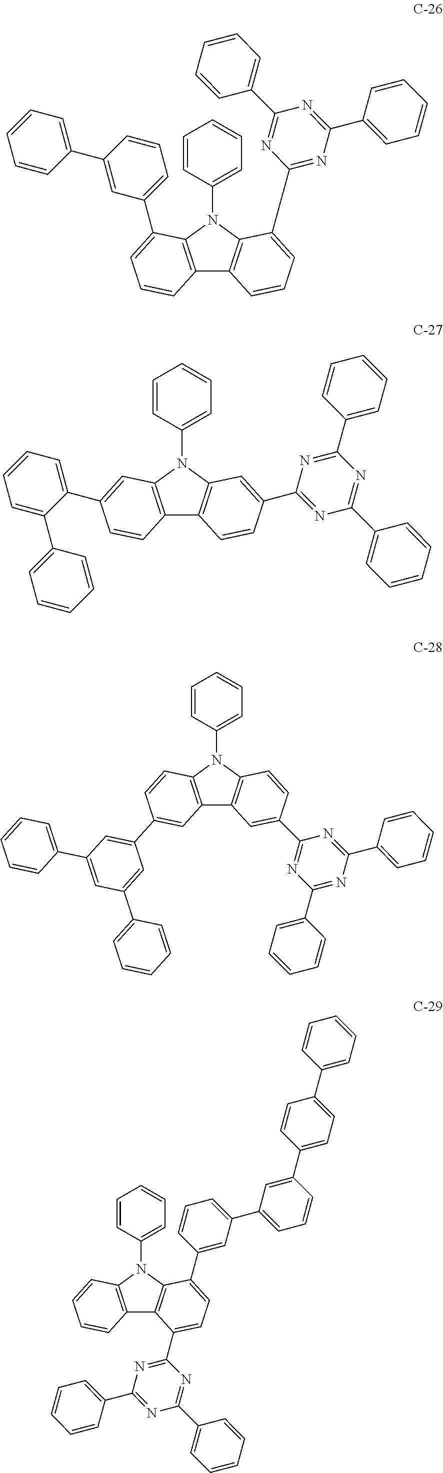 [SMILES: c1ccc(-c2cccc(-c3cccc4c5cccc(-c6nc(-c7ccccc7)nc(-c7ccccc7)n6)c5n(-c5ccccc5)c34)c2)cc1 ; c1ccc(-c2nc(-c3ccccc3)nc(-c3ccc4c5ccc(-c6ccccc6-c6ccccc6)cc5n(-c5ccccc5)c4c3)n2)cc1 ; c1ccc(-c2cc(-c3ccccc3)cc(-c3ccc4c(c3)c3cc(-c5nc(-c6ccccc6)nc(-c6ccccc6)n5)ccc3n4-c3ccccc3)c2)cc1 ; c1ccc(-c2ccc(-c3cccc(-c4cccc(-c5ccc(-c6nc(-c7ccccc7)nc(-c7ccccc7)n6)c6c7ccccc7n(-c7ccccc7)c56)c4)c3)cc2)cc1]

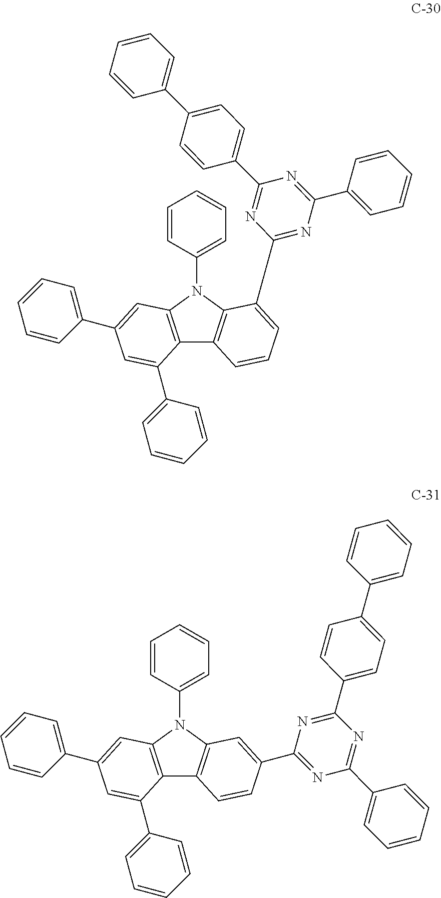 [SMILES: c1ccc(-c2ccc(-c3nc(-c4ccccc4)nc(-c4cccc5c6c(-c7ccccc7)cc(-c7ccccc7)cc6n(-c6ccccc6)c45)n3)cc2)cc1 ; c1ccc(-c2ccc(-c3nc(-c4ccccc4)nc(-c4ccc5c6c(-c7ccccc7)cc(-c7ccccc7)cc6n(-c6ccccc6)c5c4)n3)cc2)cc1]

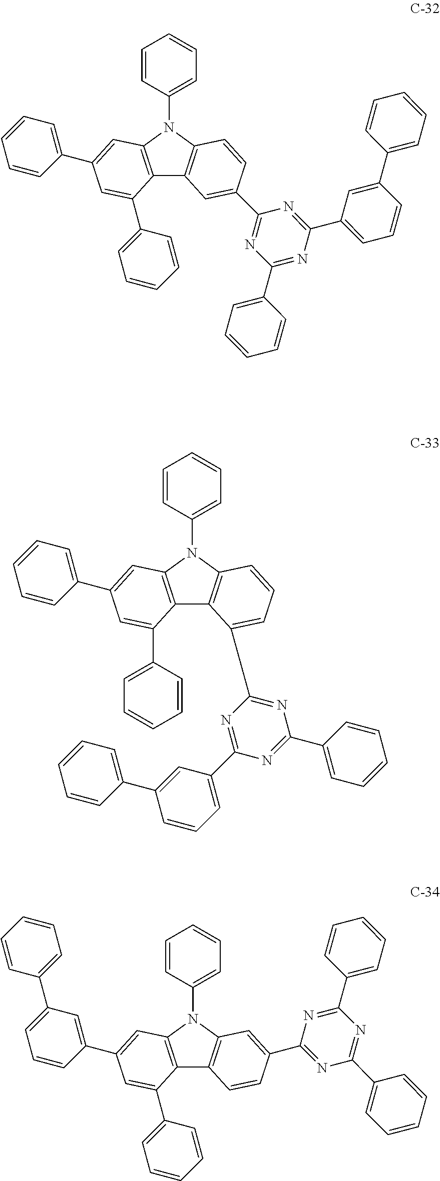 [SMILES: c1ccc(-c2cccc(-c3nc(-c4ccccc4)nc(-c4ccc5c(c4)c4c(-c6ccccc6)cc(-c6ccccc6)cc4n5-c4ccccc4)n3)c2)cc1 ; c1ccc(-c2cccc(-c3nc(-c4ccccc4)nc(-c4cccc5c4c4c(-c6ccccc6)cc(-c6ccccc6)cc4n5-c4ccccc4)n3)c2)cc1 ; c1ccc(-c2cccc(-c3cc(-c4ccccc4)c4c5ccc(-c6nc(-c7ccccc7)nc(-c7ccccc7)n6)cc5n(-c5ccccc5)c4c3)c2)cc1]

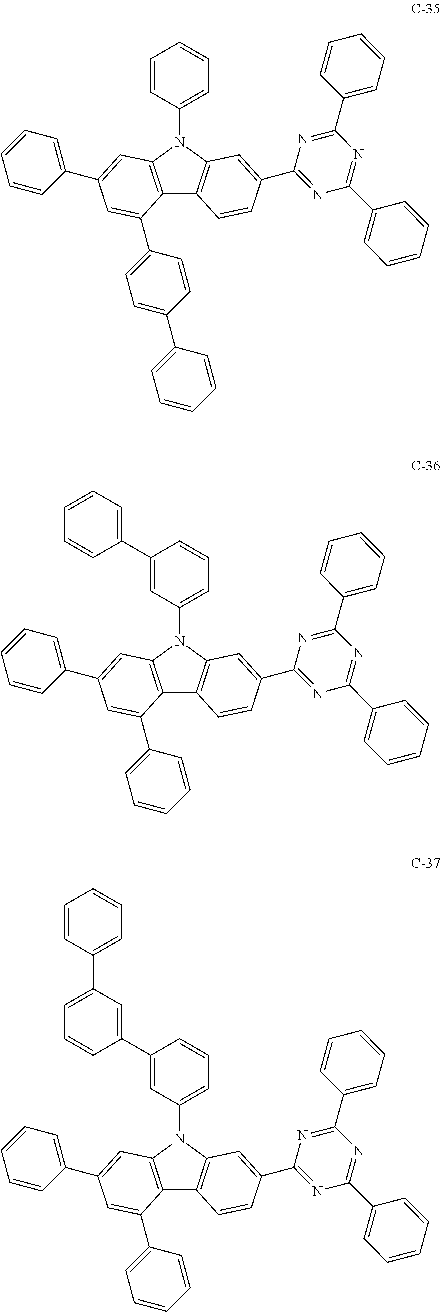 [SMILES: c1ccc(-c2ccc(-c3cc(-c4ccccc4)cc4c3c3ccc(-c5nc(-c6ccccc6)nc(-c6ccccc6)n5)cc3n4-c3ccccc3)cc2)cc1 ; c1ccc(-c2cccc(-n3c4cc(-c5nc(-c6ccccc6)nc(-c6ccccc6)n5)ccc4c4c(-c5ccccc5)cc(-c5ccccc5)cc43)c2)cc1 ; c1ccc(-c2cccc(-c3cccc(-n4c5cc(-c6nc(-c7ccccc7)nc(-c7ccccc7)n6)ccc5c5c(-c6ccccc6)cc(-c6ccccc6)cc54)c3)c2)cc1]

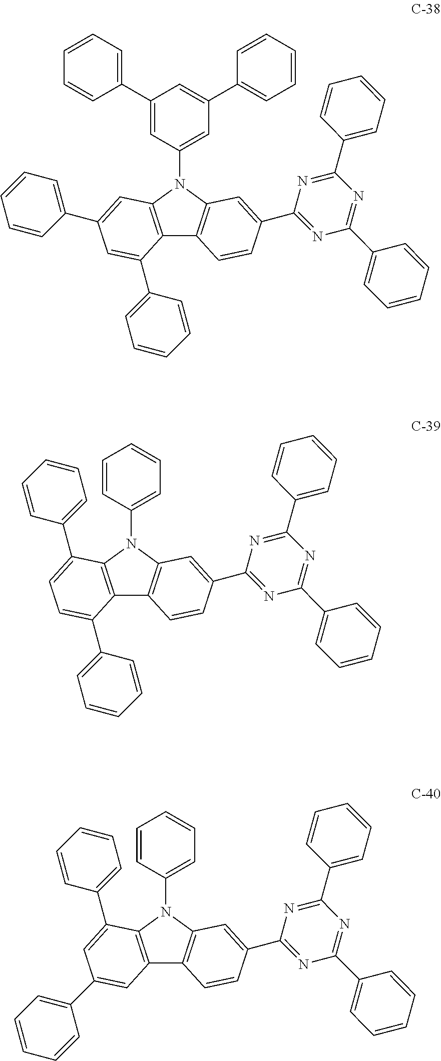 [SMILES: c1ccc(-c2cc(-c3ccccc3)cc(-n3c4cc(-c5nc(-c6ccccc6)nc(-c6ccccc6)n5)ccc4c4c(-c5ccccc5)cc(-c5ccccc5)cc43)c2)cc1 ; c1ccc(-c2nc(-c3ccccc3)nc(-c3ccc4c5c(-c6ccccc6)ccc(-c6ccccc6)c5n(-c5ccccc5)c4c3)n2)cc1 ; c1ccc(-c2cc(-c3ccccc3)c3c(c2)c2ccc(-c4nc(-c5ccccc5)nc(-c5ccccc5)n4)cc2n3-c2ccccc2)cc1]

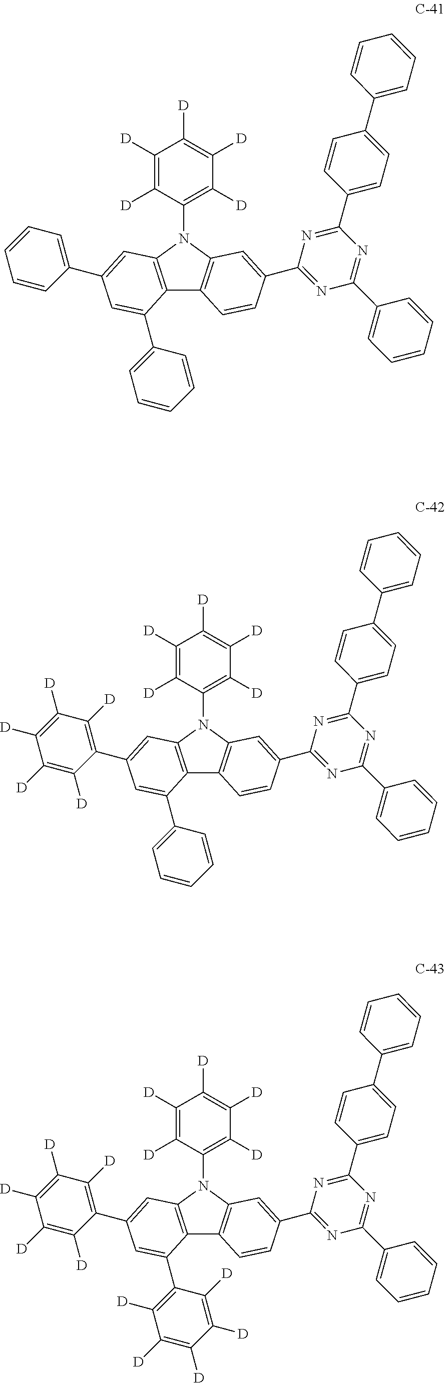 [SMILES: [2H]c1c([2H])c([2H])c(-n2c3cc(-c4nc(-c5ccccc5)nc(-c5ccc(-c6ccccc6)cc5)n4)ccc3c3c(-c4ccccc4)cc(-c4ccccc4)cc32)c([2H])c1[2H] ; [2H]c1c([2H])c([2H])c(-c2cc(-c3ccccc3)c3c4ccc(-c5nc(-c6ccccc6)nc(-c6ccc(-c7ccccc7)cc6)n5)cc4n(-c4c([2H])c([2H])c([2H])c([2H])c4[2H])c3c2)c([2H])c1[2H] ; [2H]c1c([2H])c([2H])c(-c2cc(-c3c([2H])c([2H])c([2H])c([2H])c3[2H])c3c4ccc(-c5nc(-c6ccccc6)nc(-c6ccc(-c7ccccc7)cc6)n5)cc4n(-c4c([2H])c([2H])c([2H])c([2H])c4[2H])c3c2)c([2H])c1[2H]]

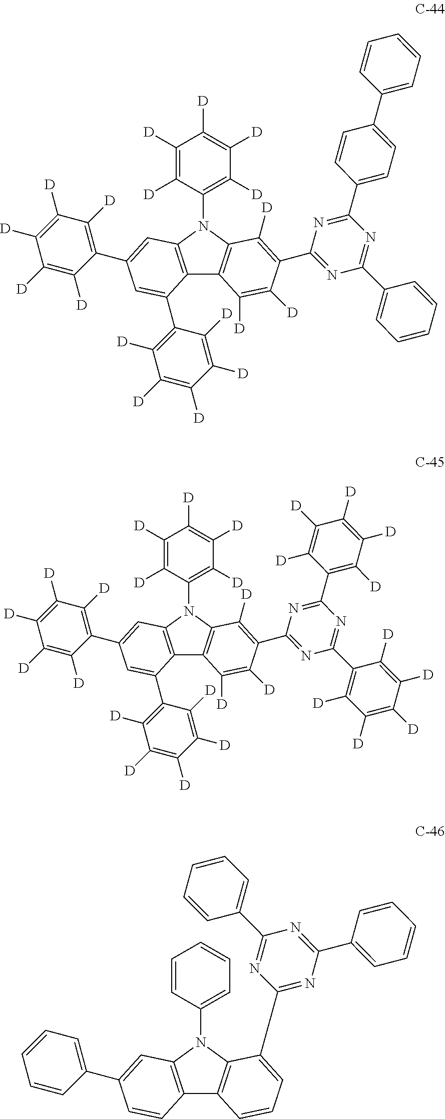

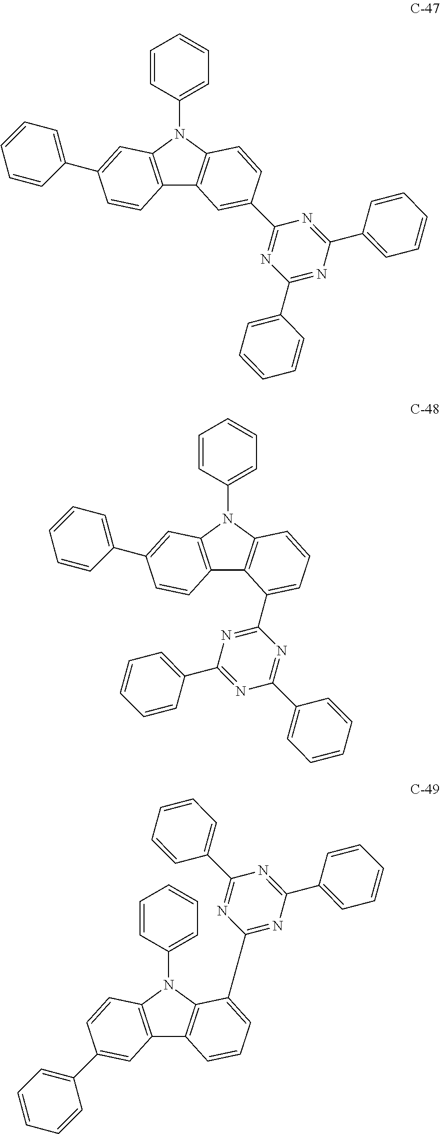 [SMILES: c1ccc(-c2ccc3c4cc(-c5nc(-c6ccccc6)nc(-c6ccccc6)n5)ccc4n(-c4ccccc4)c3c2)cc1 ; c1ccc(-c2ccc3c4c(-c5nc(-c6ccccc6)nc(-c6ccccc6)n5)cccc4n(-c4ccccc4)c3c2)cc1 ; c1ccc(-c2ccc3c(c2)c2cccc(-c4nc(-c5ccccc5)nc(-c5ccccc5)n4)c2n3-c2ccccc2)cc1]

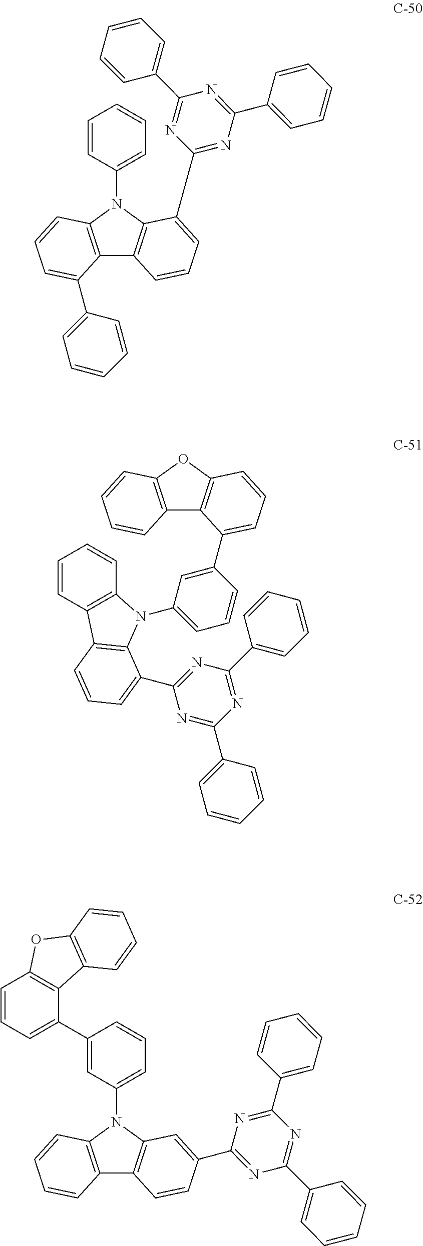 [SMILES: c1ccc(-c2nc(-c3ccccc3)nc(-c3cccc4c5c(-c6ccccc6)cccc5n(-c5ccccc5)c34)n2)cc1 ; c1ccc(-c2nc(-c3ccccc3)nc(-c3cccc4c5ccccc5n(-c5cccc(-c6cccc7oc8ccccc8c67)c5)c34)n2)cc1 ; c1ccc(-c2nc(-c3ccccc3)nc(-c3ccc4c5ccccc5n(-c5cccc(-c6cccc7oc8ccccc8c67)c5)c4c3)n2)cc1]

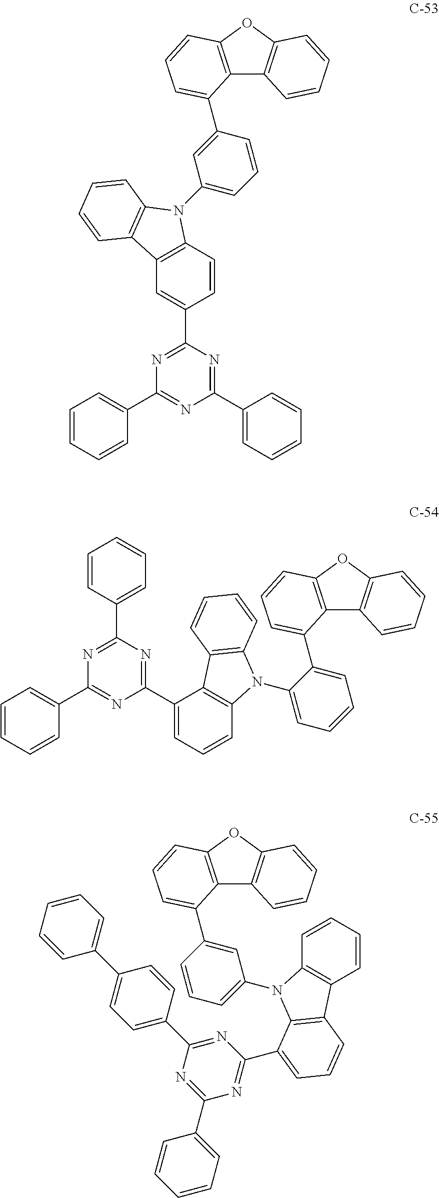 [SMILES: c1ccc(-c2nc(-c3ccccc3)nc(-c3ccc4c(c3)c3ccccc3n4-c3cccc(-c4cccc5oc6ccccc6c45)c3)n2)cc1 ; c1ccc(-c2nc(-c3ccccc3)nc(-c3cccc4c3c3ccccc3n4-c3ccccc3-c3cccc4oc5ccccc5c34)n2)cc1 ; c1ccc(-c2ccc(-c3nc(-c4ccccc4)nc(-c4cccc5c6ccccc6n(-c6cccc(-c7cccc8oc9ccccc9c78)c6)c45)n3)cc2)cc1]

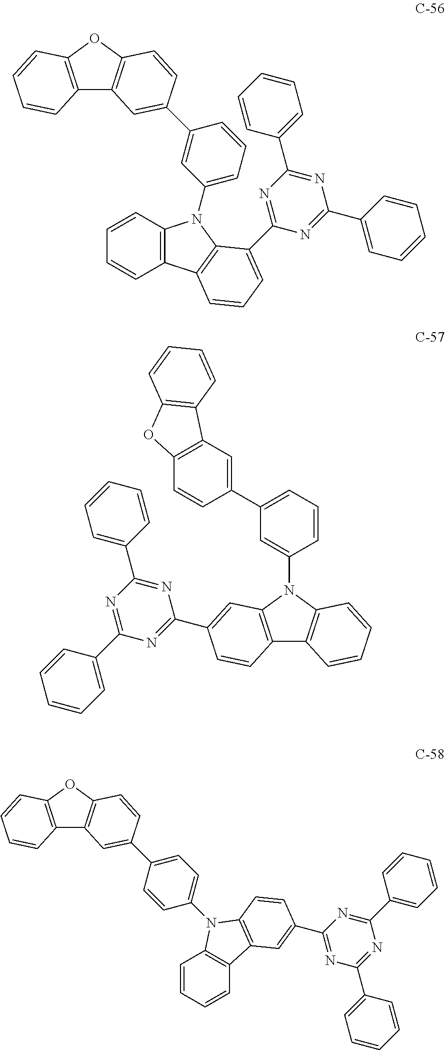 [SMILES: c1ccc(-c2nc(-c3ccccc3)nc(-c3cccc4c5ccccc5n(-c5cccc(-c6ccc7oc8ccccc8c7c6)c5)c34)n2)cc1 ; c1ccc(-c2nc(-c3ccccc3)nc(-c3ccc4c5ccccc5n(-c5cccc(-c6ccc7oc8ccccc8c7c6)c5)c4c3)n2)cc1 ; c1ccc(-c2nc(-c3ccccc3)nc(-c3ccc4c(c3)c3ccccc3n4-c3ccc(-c4ccc5oc6ccccc6c5c4)cc3)n2)cc1]

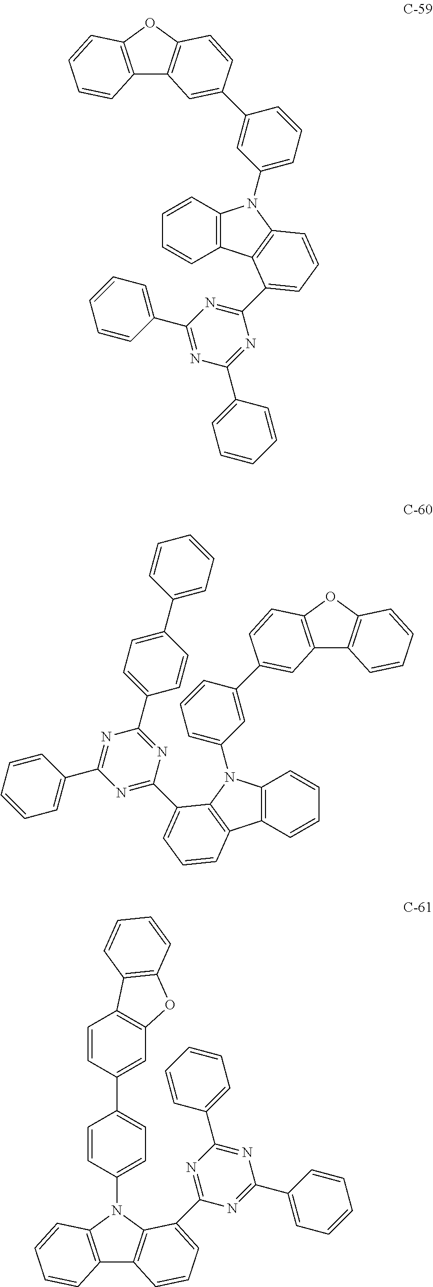 [SMILES: c1ccc(-c2nc(-c3ccccc3)nc(-c3cccc4c3c3ccccc3n4-c3cccc(-c4ccc5oc6ccccc6c5c4)c3)n2)cc1 ; c1ccc(-c2ccc(-c3nc(-c4ccccc4)nc(-c4cccc5c6ccccc6n(-c6cccc(-c7ccc8oc9ccccc9c8c7)c6)c45)n3)cc2)cc1 ; c1ccc(-c2nc(-c3ccccc3)nc(-c3cccc4c5ccccc5n(-c5ccc(-c6ccc7c(c6)oc6ccccc67)cc5)c34)n2)cc1]

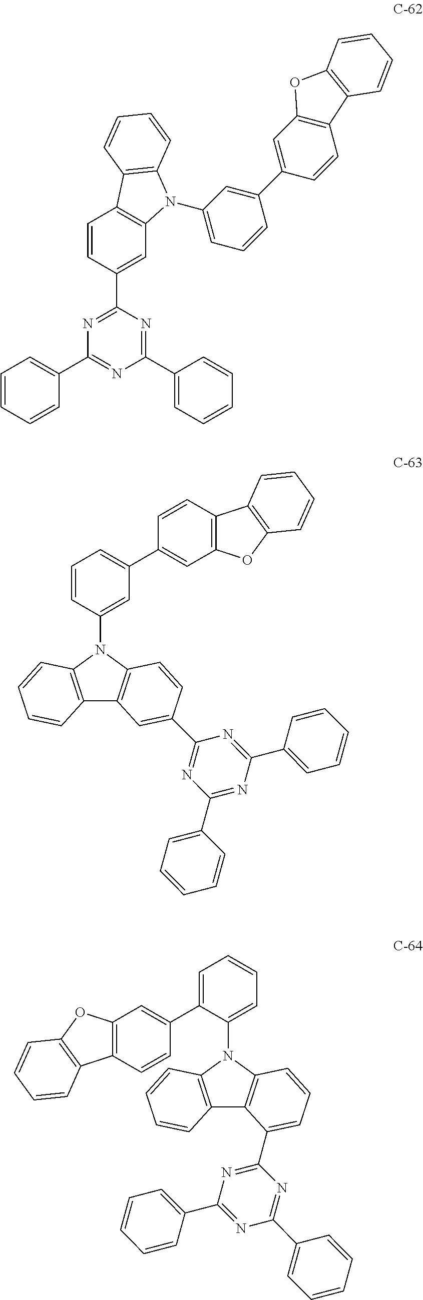 [SMILES: c1ccc(-c2nc(-c3ccccc3)nc(-c3ccc4c5ccccc5n(-c5cccc(-c6ccc7c(c6)oc6ccccc67)c5)c4c3)n2)cc1 ; c1ccc(-c2nc(-c3ccccc3)nc(-c3ccc4c(c3)c3ccccc3n4-c3cccc(-c4ccc5c(c4)oc4ccccc45)c3)n2)cc1 ; c1ccc(-c2nc(-c3ccccc3)nc(-c3cccc4c3c3ccccc3n4-c3ccccc3-c3ccc4c(c3)oc3ccccc34)n2)cc1]

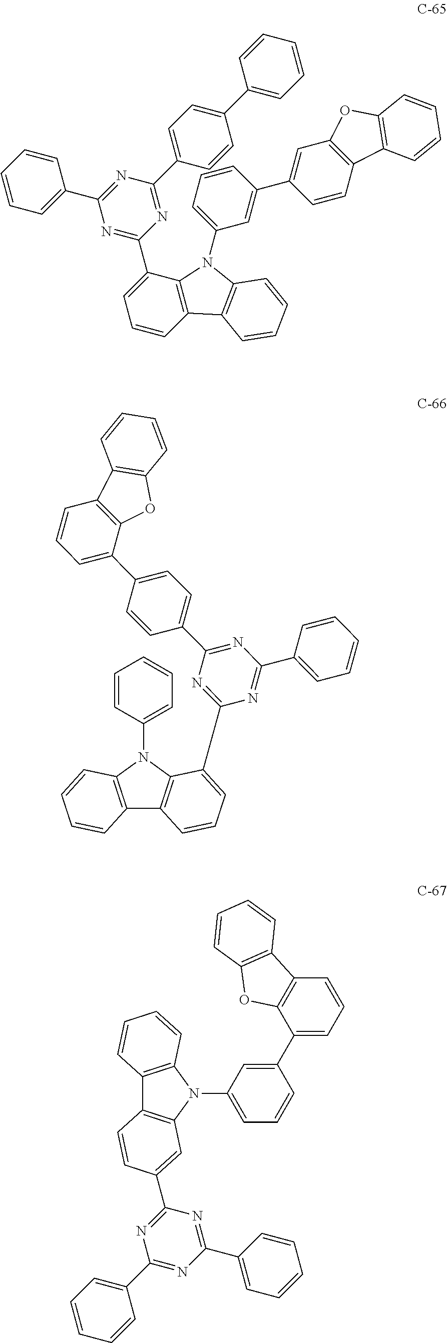 [SMILES: c1ccc(-c2ccc(-c3nc(-c4ccccc4)nc(-c4cccc5c6ccccc6n(-c6cccc(-c7ccc8c(c7)oc7ccccc78)c6)c45)n3)cc2)cc1 ; c1ccc(-c2nc(-c3ccc(-c4cccc5c4oc4ccccc45)cc3)nc(-c3cccc4c5ccccc5n(-c5ccccc5)c34)n2)cc1 ; c1ccc(-c2nc(-c3ccccc3)nc(-c3ccc4c5ccccc5n(-c5cccc(-c6cccc7c6oc6ccccc67)c5)c4c3)n2)cc1]

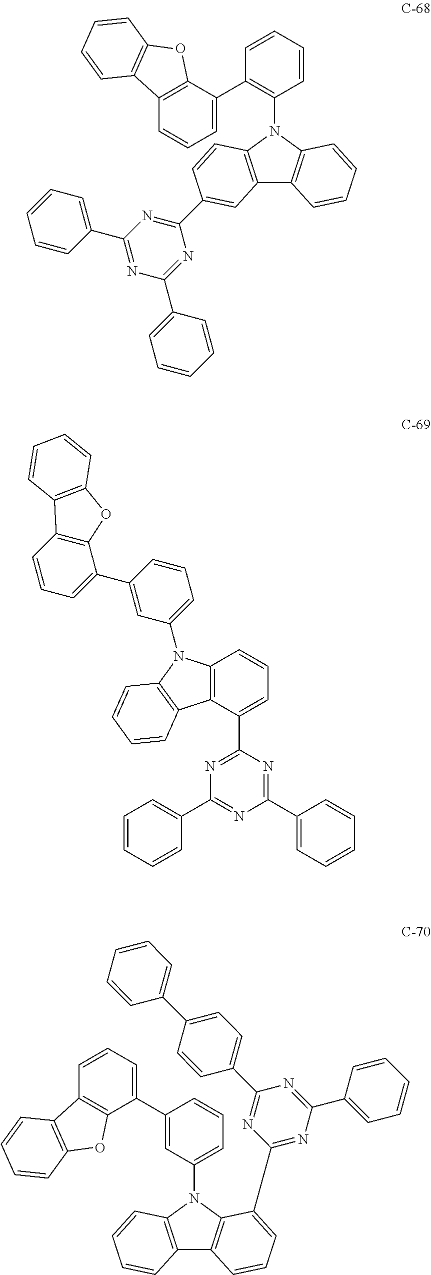 [SMILES: c1ccc(-c2nc(-c3ccccc3)nc(-c3ccc4c(c3)c3ccccc3n4-c3ccccc3-c3cccc4c3oc3ccccc34)n2)cc1 ; c1ccc(-c2nc(-c3ccccc3)nc(-c3cccc4c3c3ccccc3n4-c3cccc(-c4cccc5c4oc4ccccc45)c3)n2)cc1 ; c1ccc(-c2ccc(-c3nc(-c4ccccc4)nc(-c4cccc5c6ccccc6n(-c6cccc(-c7cccc8c7oc7ccccc78)c6)c45)n3)cc2)cc1]

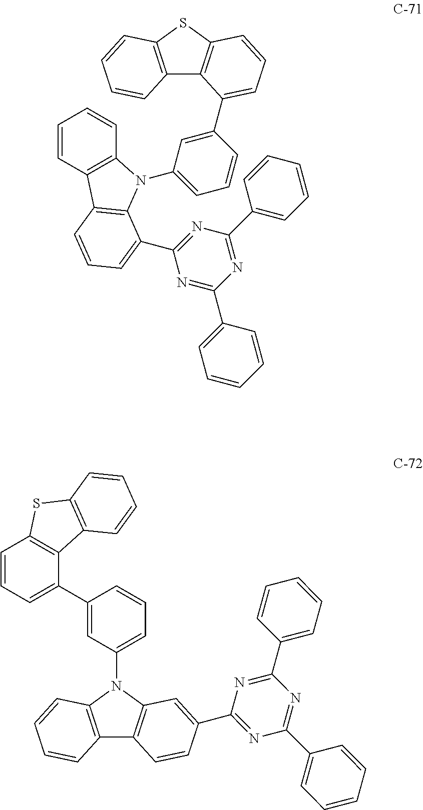 [SMILES: c1ccc(-c2nc(-c3ccccc3)nc(-c3cccc4c5ccccc5n(-c5cccc(-c6cccc7sc8ccccc8c67)c5)c34)n2)cc1 ; c1ccc(-c2nc(-c3ccccc3)nc(-c3ccc4c5ccccc5n(-c5cccc(-c6cccc7sc8ccccc8c67)c5)c4c3)n2)cc1]

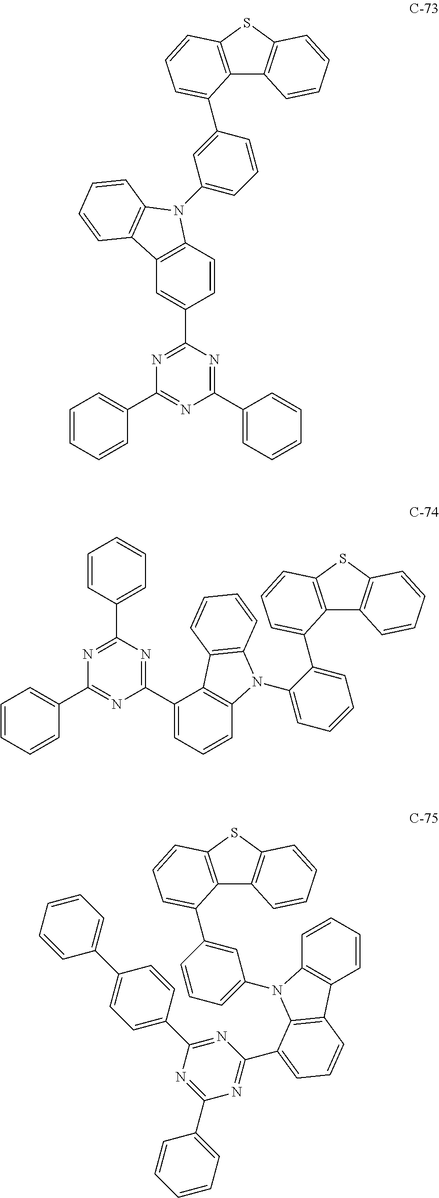 [SMILES: c1ccc(-c2nc(-c3ccccc3)nc(-c3ccc4c(c3)c3ccccc3n4-c3cccc(-c4cccc5sc6ccccc6c45)c3)n2)cc1 ; c1ccc(-c2nc(-c3ccccc3)nc(-c3cccc4c3c3ccccc3n4-c3ccccc3-c3cccc4sc5ccccc5c34)n2)cc1 ; c1ccc(-c2ccc(-c3nc(-c4ccccc4)nc(-c4cccc5c6ccccc6n(-c6cccc(-c7cccc8sc9ccccc9c78)c6)c45)n3)cc2)cc1]

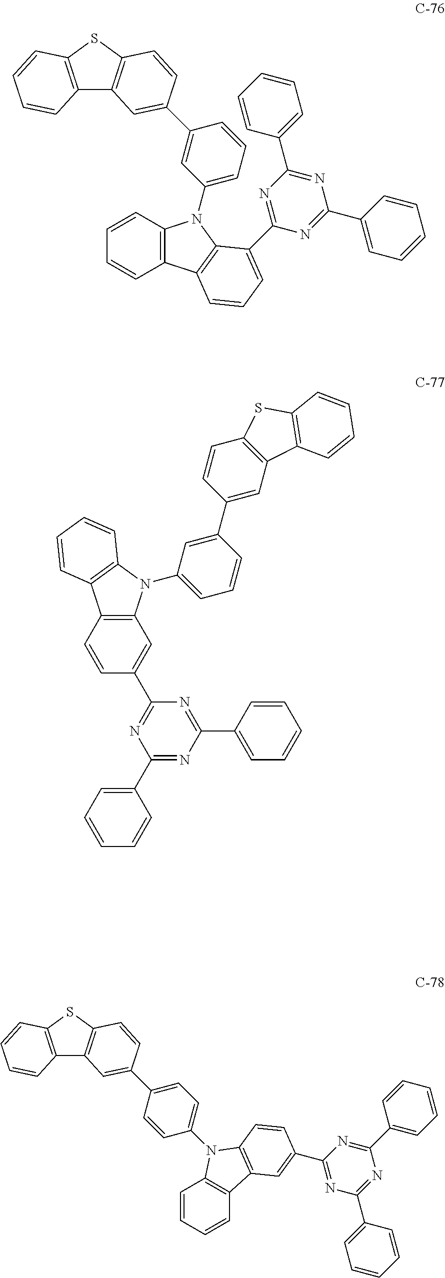 [SMILES: c1ccc(-c2nc(-c3ccccc3)nc(-c3cccc4c5ccccc5n(-c5cccc(-c6ccc7sc8ccccc8c7c6)c5)c34)n2)cc1 ; c1ccc(-c2nc(-c3ccccc3)nc(-c3ccc4c5ccccc5n(-c5cccc(-c6ccc7sc8ccccc8c7c6)c5)c4c3)n2)cc1 ; c1ccc(-c2nc(-c3ccccc3)nc(-c3ccc4c(c3)c3ccccc3n4-c3ccc(-c4ccc5sc6ccccc6c5c4)cc3)n2)cc1]

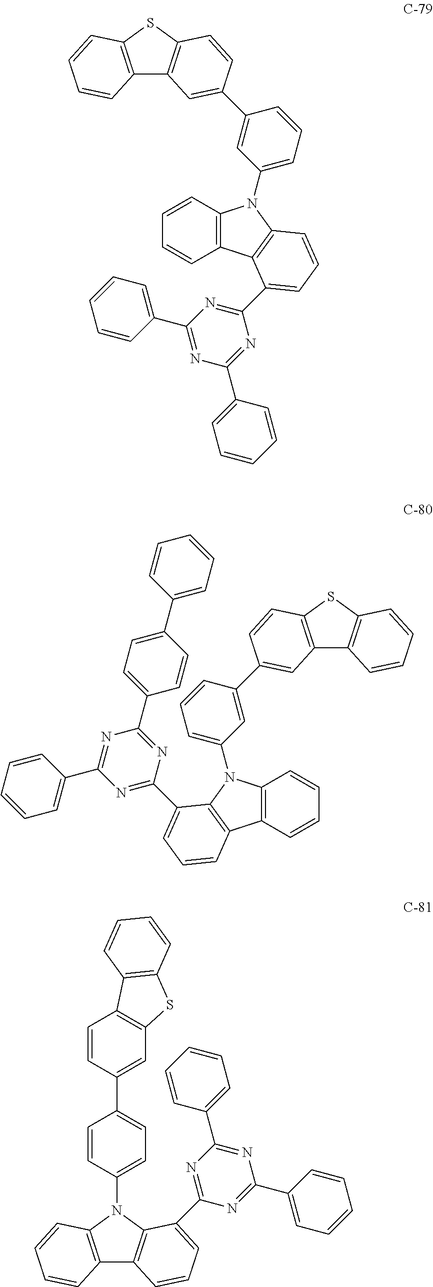 [SMILES: c1ccc(-c2nc(-c3ccccc3)nc(-c3cccc4c3c3ccccc3n4-c3cccc(-c4ccc5sc6ccccc6c5c4)c3)n2)cc1 ; c1ccc(-c2ccc(-c3nc(-c4ccccc4)nc(-c4cccc5c6ccccc6n(-c6cccc(-c7ccc8sc9ccccc9c8c7)c6)c45)n3)cc2)cc1 ; c1ccc(-c2nc(-c3ccccc3)nc(-c3cccc4c5ccccc5n(-c5ccc(-c6ccc7c(c6)sc6ccccc67)cc5)c34)n2)cc1]

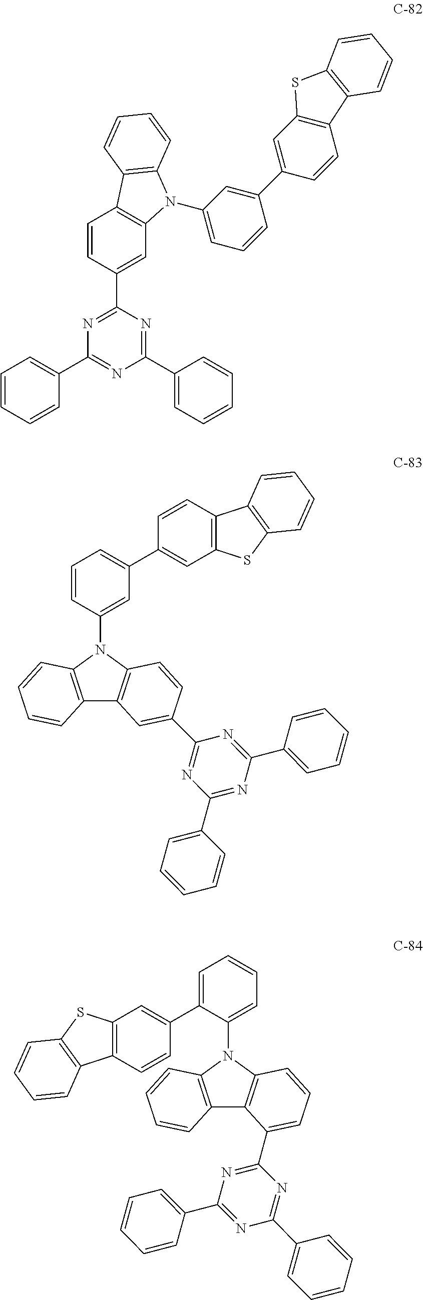 [SMILES: c1ccc(-c2nc(-c3ccccc3)nc(-c3ccc4c5ccccc5n(-c5cccc(-c6ccc7c(c6)sc6ccccc67)c5)c4c3)n2)cc1 ; c1ccc(-c2nc(-c3ccccc3)nc(-c3ccc4c(c3)c3ccccc3n4-c3cccc(-c4ccc5c(c4)sc4ccccc45)c3)n2)cc1 ; c1ccc(-c2nc(-c3ccccc3)nc(-c3cccc4c3c3ccccc3n4-c3ccccc3-c3ccc4c(c3)sc3ccccc34)n2)cc1]

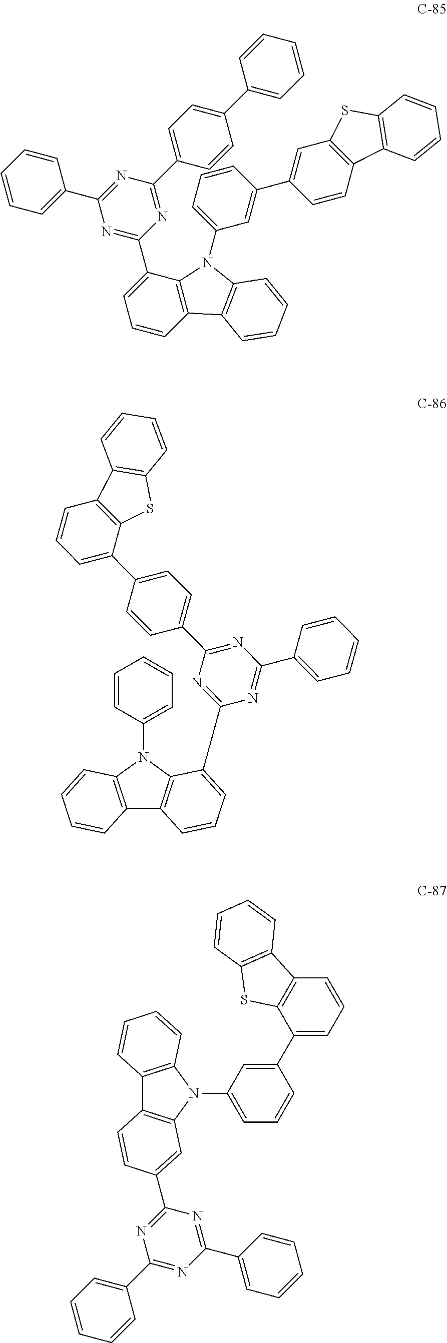 [SMILES: c1ccc(-c2ccc(-c3nc(-c4ccccc4)nc(-c4cccc5c6ccccc6n(-c6cccc(-c7ccc8c(c7)sc7ccccc78)c6)c45)n3)cc2)cc1 ; c1ccc(-c2nc(-c3ccc(-c4cccc5c4sc4ccccc45)cc3)nc(-c3cccc4c5ccccc5n(-c5ccccc5)c34)n2)cc1 ; c1ccc(-c2nc(-c3ccccc3)nc(-c3ccc4c5ccccc5n(-c5cccc(-c6cccc7c6sc6ccccc67)c5)c4c3)n2)cc1]

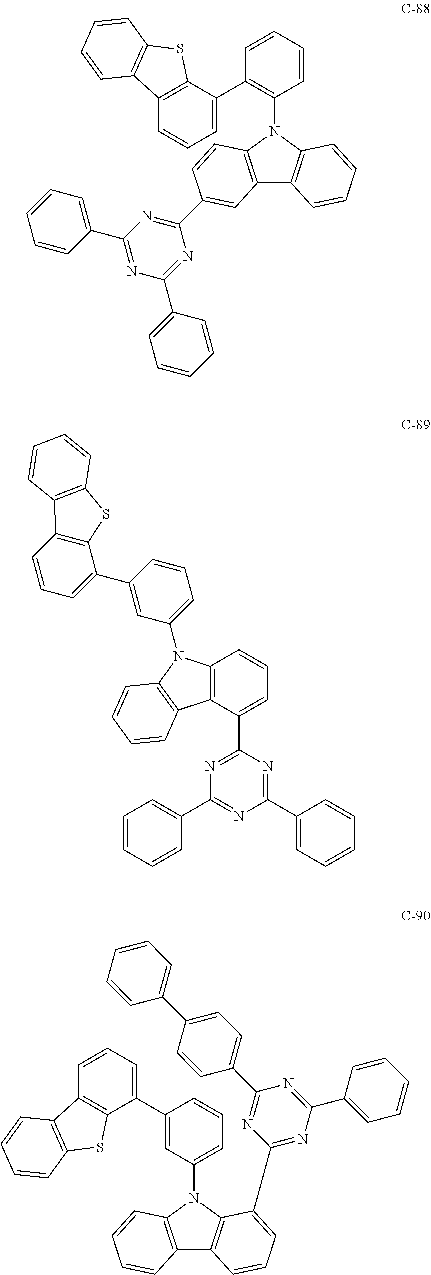 [SMILES: c1ccc(-c2nc(-c3ccccc3)nc(-c3ccc4c(c3)c3ccccc3n4-c3ccccc3-c3cccc4c3sc3ccccc34)n2)cc1 ; c1ccc(-c2nc(-c3ccccc3)nc(-c3cccc4c3c3ccccc3n4-c3cccc(-c4cccc5c4sc4ccccc45)c3)n2)cc1 ; c1ccc(-c2ccc(-c3nc(-c4ccccc4)nc(-c4cccc5c6ccccc6n(-c6cccc(-c7cccc8c7sc7ccccc78)c6)c45)n3)cc2)cc1]

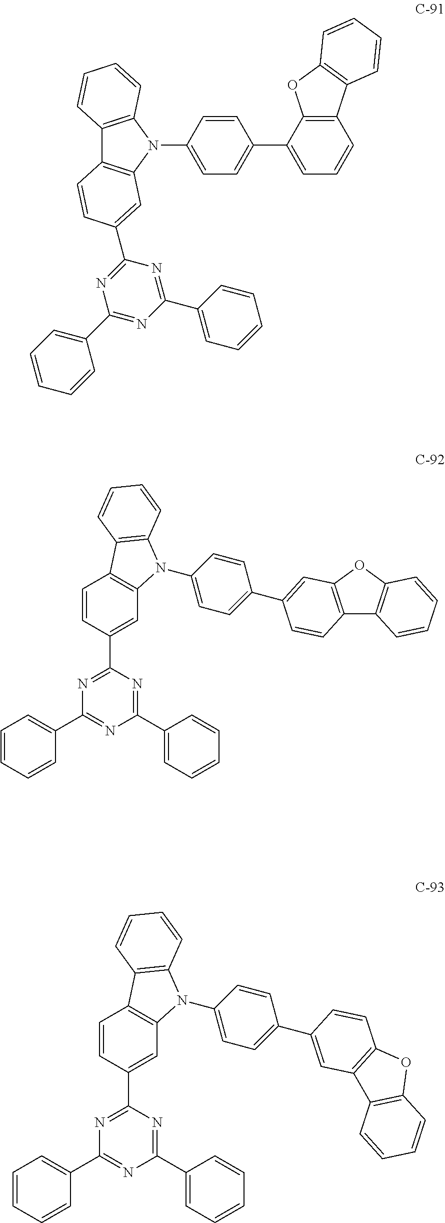 [SMILES: c1ccc(-c2nc(-c3ccccc3)nc(-c3ccc4c5ccccc5n(-c5ccc(-c6cccc7c6oc6ccccc67)cc5)c4c3)n2)cc1 ; c1ccc(-c2nc(-c3ccccc3)nc(-c3ccc4c5ccccc5n(-c5ccc(-c6ccc7c(c6)oc6ccccc67)cc5)c4c3)n2)cc1 ; c1ccc(-c2nc(-c3ccccc3)nc(-c3ccc4c5ccccc5n(-c5ccc(-c6ccc7oc8ccccc8c7c6)cc5)c4c3)n2)cc1]

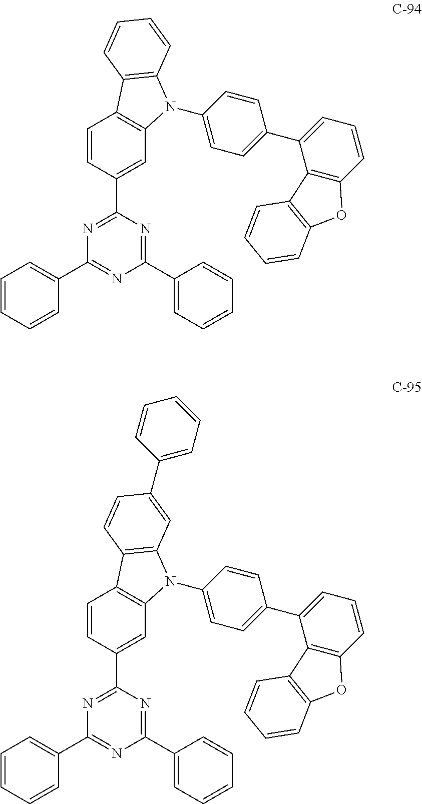 [SMILES: c1ccc(-c2nc(-c3ccccc3)nc(-c3ccc4c5ccccc5n(-c5ccc(-c6cccc7oc8ccccc8c67)cc5)c4c3)n2)cc1 ; c1ccc(-c2ccc3c4ccc(-c5nc(-c6ccccc6)nc(-c6ccccc6)n5)cc4n(-c4ccc(-c5cccc6oc7ccccc7c56)cc4)c3c2)cc1]

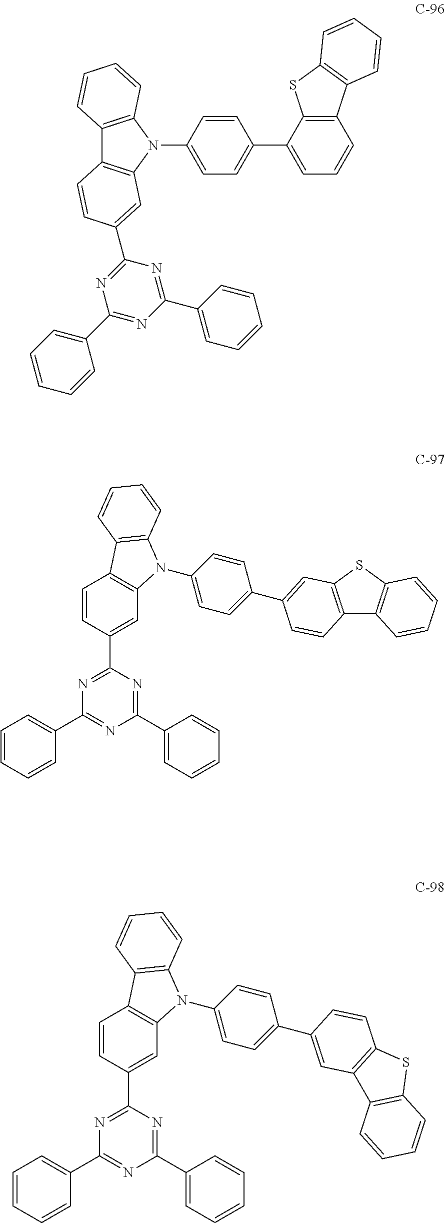 [SMILES: c1ccc(-c2nc(-c3ccccc3)nc(-c3ccc4c5ccccc5n(-c5ccc(-c6cccc7c6sc6ccccc67)cc5)c4c3)n2)cc1 ; c1ccc(-c2nc(-c3ccccc3)nc(-c3ccc4c5ccccc5n(-c5ccc(-c6ccc7c(c6)sc6ccccc67)cc5)c4c3)n2)cc1 ; c1ccc(-c2nc(-c3ccccc3)nc(-c3ccc4c5ccccc5n(-c5ccc(-c6ccc7sc8ccccc8c7c6)cc5)c4c3)n2)cc1]

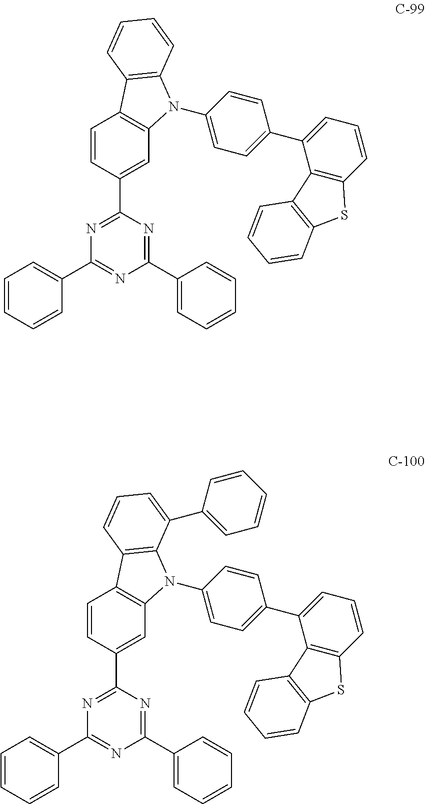 [SMILES: c1ccc(-c2nc(-c3ccccc3)nc(-c3ccc4c5ccccc5n(-c5ccc(-c6cccc7sc8ccccc8c67)cc5)c4c3)n2)cc1 ; c1ccc(-c2nc(-c3ccccc3)nc(-c3ccc4c5cccc(-c6ccccc6)c5n(-c5ccc(-c6cccc7sc8ccccc8c67)cc5)c4c3)n2)cc1]

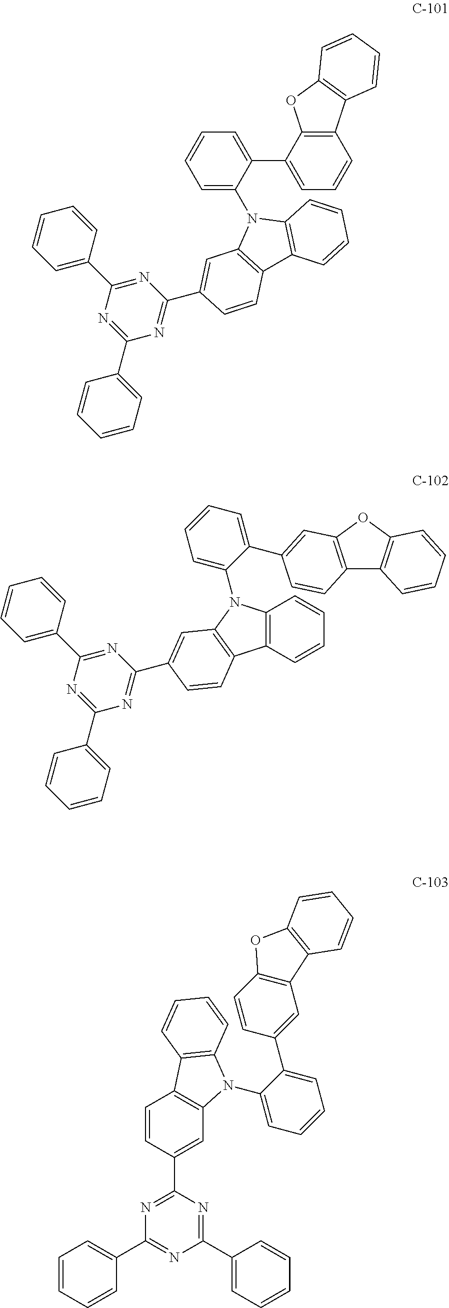 [SMILES: c1ccc(-c2nc(-c3ccccc3)nc(-c3ccc4c5ccccc5n(-c5ccccc5-c5cccc6c5oc5ccccc56)c4c3)n2)cc1 ; c1ccc(-c2nc(-c3ccccc3)nc(-c3ccc4c5ccccc5n(-c5ccccc5-c5ccc6c(c5)oc5ccccc56)c4c3)n2)cc1 ; c1ccc(-c2nc(-c3ccccc3)nc(-c3ccc4c5ccccc5n(-c5ccccc5-c5ccc6oc7ccccc7c6c5)c4c3)n2)cc1]

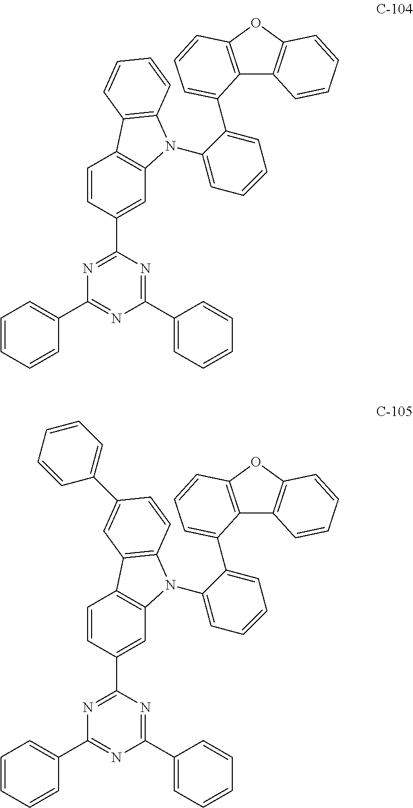 [SMILES: c1ccc(-c2nc(-c3ccccc3)nc(-c3ccc4c5ccccc5n(-c5ccccc5-c5cccc6oc7ccccc7c56)c4c3)n2)cc1 ; c1ccc(-c2ccc3c(c2)c2ccc(-c4nc(-c5ccccc5)nc(-c5ccccc5)n4)cc2n3-c2ccccc2-c2cccc3oc4ccccc4c23)cc1]

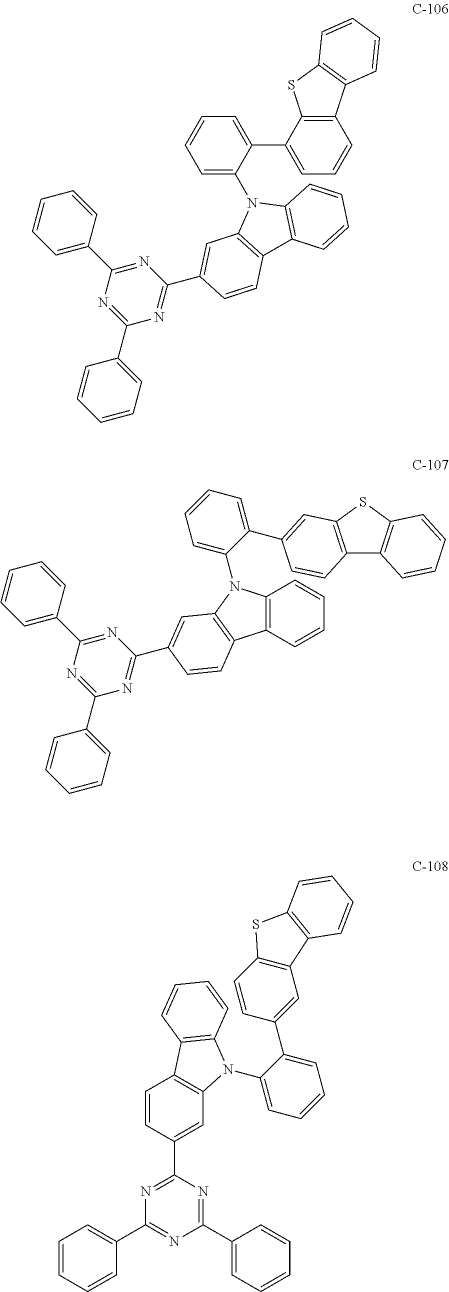 [SMILES: c1ccc(-c2nc(-c3ccccc3)nc(-c3ccc4c5ccccc5n(-c5ccccc5-c5cccc6c5sc5ccccc56)c4c3)n2)cc1 ; c1ccc(-c2nc(-c3ccccc3)nc(-c3ccc4c5ccccc5n(-c5ccccc5-c5ccc6c(c5)sc5ccccc56)c4c3)n2)cc1 ; c1ccc(-c2nc(-c3ccccc3)nc(-c3ccc4c5ccccc5n(-c5ccccc5-c5ccc6sc7ccccc7c6c5)c4c3)n2)cc1]

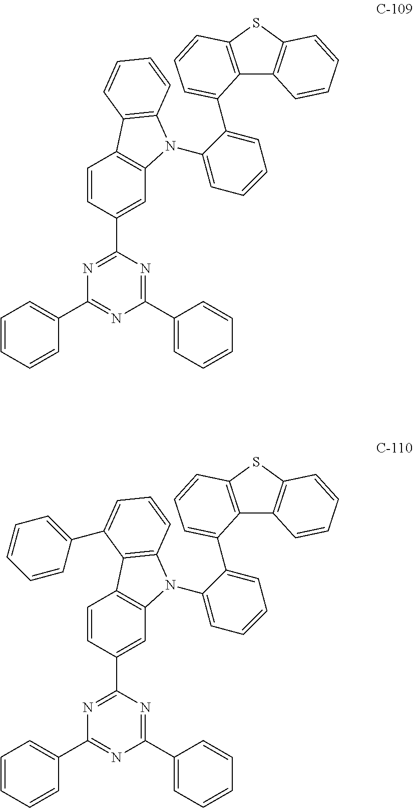 [SMILES: c1ccc(-c2nc(-c3ccccc3)nc(-c3ccc4c5ccccc5n(-c5ccccc5-c5cccc6sc7ccccc7c56)c4c3)n2)cc1 ; c1ccc(-c2nc(-c3ccccc3)nc(-c3ccc4c5c(-c6ccccc6)cccc5n(-c5ccccc5-c5cccc6sc7ccccc7c56)c4c3)n2)cc1]

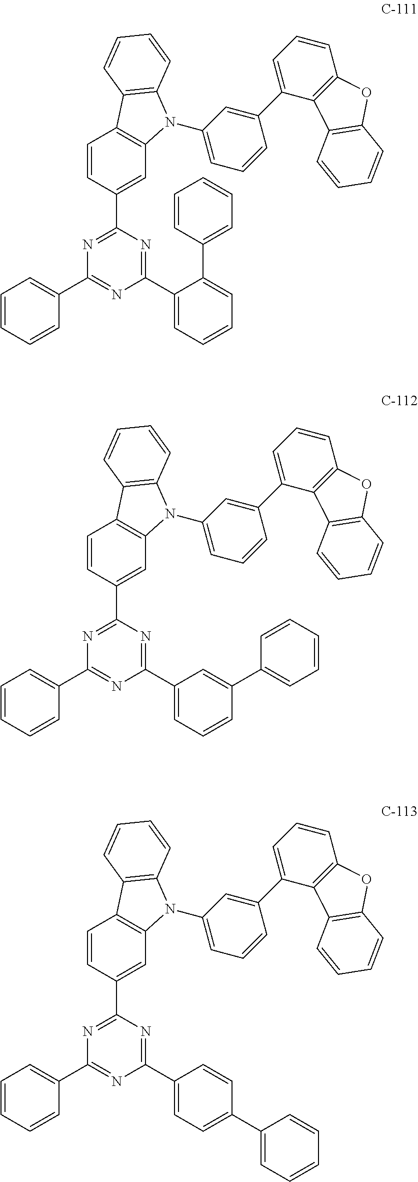 [SMILES: c1ccc(-c2nc(-c3ccc4c5ccccc5n(-c5cccc(-c6cccc7oc8ccccc8c67)c5)c4c3)nc(-c3ccccc3-c3ccccc3)n2)cc1 ; c1ccc(-c2cccc(-c3nc(-c4ccccc4)nc(-c4ccc5c6ccccc6n(-c6cccc(-c7cccc8oc9ccccc9c78)c6)c5c4)n3)c2)cc1 ; c1ccc(-c2ccc(-c3nc(-c4ccccc4)nc(-c4ccc5c6ccccc6n(-c6cccc(-c7cccc8oc9ccccc9c78)c6)c5c4)n3)cc2)cc1]

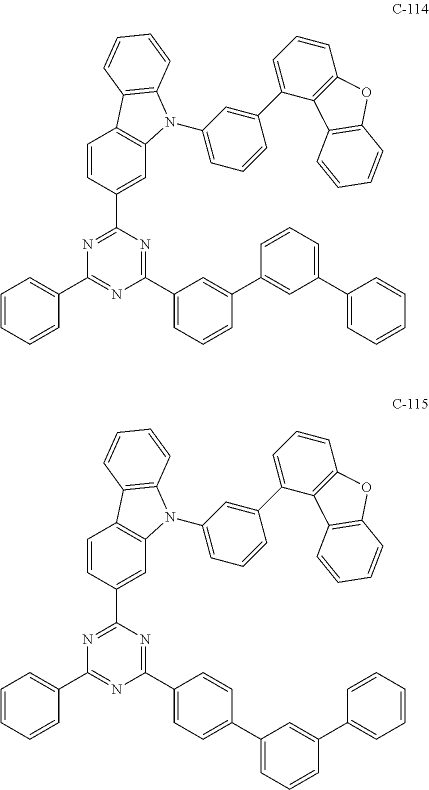 [SMILES: c1ccc(-c2cccc(-c3cccc(-c4nc(-c5ccccc5)nc(-c5ccc6c7ccccc7n(-c7cccc(-c8cccc9oc%10ccccc%10c89)c7)c6c5)n4)c3)c2)cc1 ; c1ccc(-c2cccc(-c3ccc(-c4nc(-c5ccccc5)nc(-c5ccc6c7ccccc7n(-c7cccc(-c8cccc9oc%10ccccc%10c89)c7)c6c5)n4)cc3)c2)cc1]

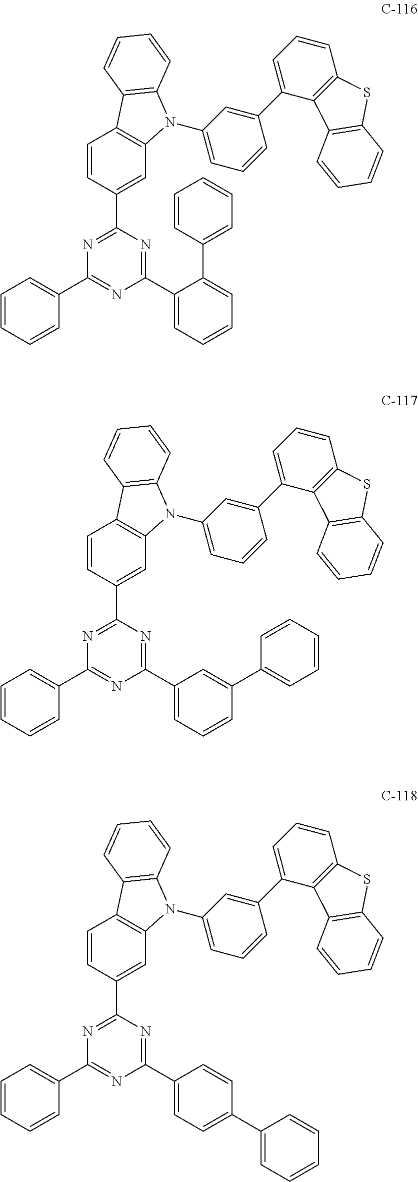 [SMILES: c1ccc(-c2nc(-c3ccc4c5ccccc5n(-c5cccc(-c6cccc7sc8ccccc8c67)c5)c4c3)nc(-c3ccccc3-c3ccccc3)n2)cc1 ; c1ccc(-c2cccc(-c3nc(-c4ccccc4)nc(-c4ccc5c6ccccc6n(-c6cccc(-c7cccc8sc9ccccc9c78)c6)c5c4)n3)c2)cc1 ; c1ccc(-c2ccc(-c3nc(-c4ccccc4)nc(-c4ccc5c6ccccc6n(-c6cccc(-c7cccc8sc9ccccc9c78)c6)c5c4)n3)cc2)cc1]

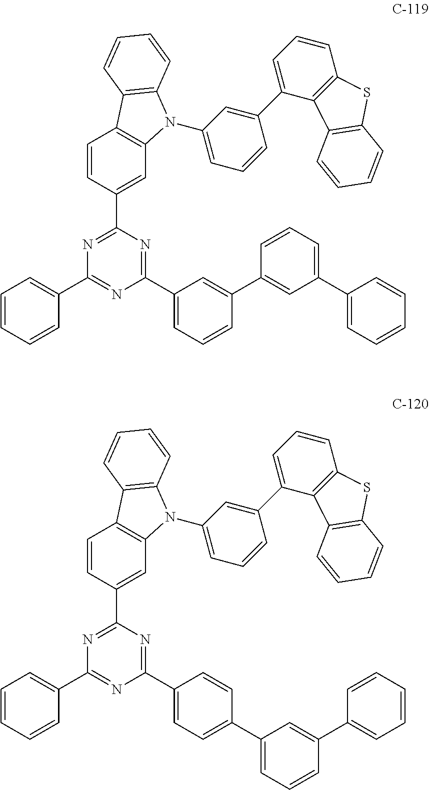 [SMILES: c1ccc(-c2cccc(-c3cccc(-c4nc(-c5ccccc5)nc(-c5ccc6c7ccccc7n(-c7cccc(-c8cccc9sc%10ccccc%10c89)c7)c6c5)n4)c3)c2)cc1 ; c1ccc(-c2cccc(-c3ccc(-c4nc(-c5ccccc5)nc(-c5ccc6c7ccccc7n(-c7cccc(-c8cccc9sc%10ccccc%10c89)c7)c6c5)n4)cc3)c2)cc1]

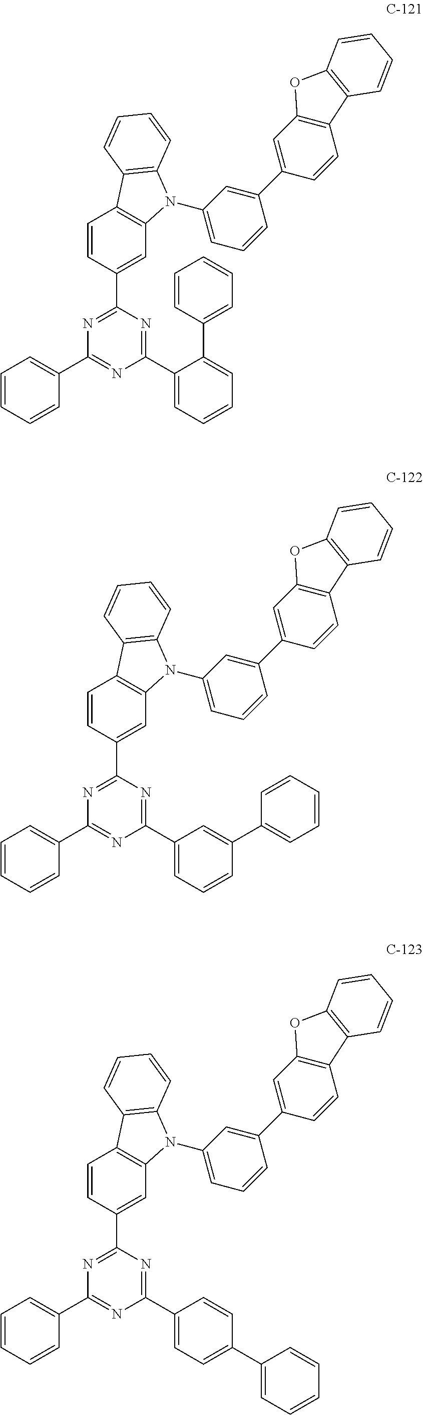 [SMILES: c1ccc(-c2nc(-c3ccc4c5ccccc5n(-c5cccc(-c6ccc7c(c6)oc6ccccc67)c5)c4c3)nc(-c3ccccc3-c3ccccc3)n2)cc1 ; c1ccc(-c2cccc(-c3nc(-c4ccccc4)nc(-c4ccc5c6ccccc6n(-c6cccc(-c7ccc8c(c7)oc7ccccc78)c6)c5c4)n3)c2)cc1 ; c1ccc(-c2ccc(-c3nc(-c4ccccc4)nc(-c4ccc5c6ccccc6n(-c6cccc(-c7ccc8c(c7)oc7ccccc78)c6)c5c4)n3)cc2)cc1]

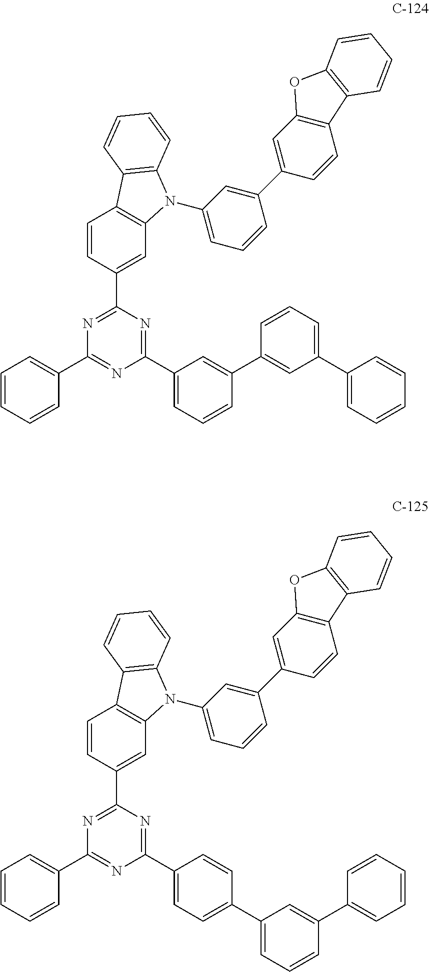 [SMILES: c1ccc(-c2cccc(-c3cccc(-c4nc(-c5ccccc5)nc(-c5ccc6c7ccccc7n(-c7cccc(-c8ccc9c(c8)oc8ccccc89)c7)c6c5)n4)c3)c2)cc1 ; c1ccc(-c2cccc(-c3ccc(-c4nc(-c5ccccc5)nc(-c5ccc6c7ccccc7n(-c7cccc(-c8ccc9c(c8)oc8ccccc89)c7)c6c5)n4)cc3)c2)cc1]

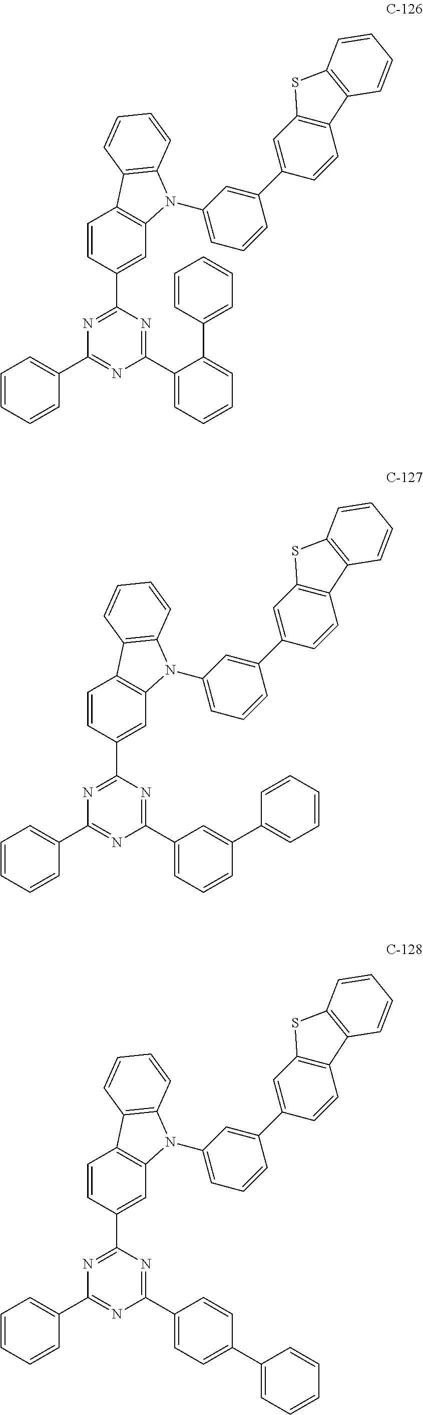 [SMILES: c1ccc(-c2nc(-c3ccc4c5ccccc5n(-c5cccc(-c6ccc7c(c6)sc6ccccc67)c5)c4c3)nc(-c3ccccc3-c3ccccc3)n2)cc1 ; c1ccc(-c2cccc(-c3nc(-c4ccccc4)nc(-c4ccc5c6ccccc6n(-c6cccc(-c7ccc8c(c7)sc7ccccc78)c6)c5c4)n3)c2)cc1 ; c1ccc(-c2ccc(-c3nc(-c4ccccc4)nc(-c4ccc5c6ccccc6n(-c6cccc(-c7ccc8c(c7)sc7ccccc78)c6)c5c4)n3)cc2)cc1]

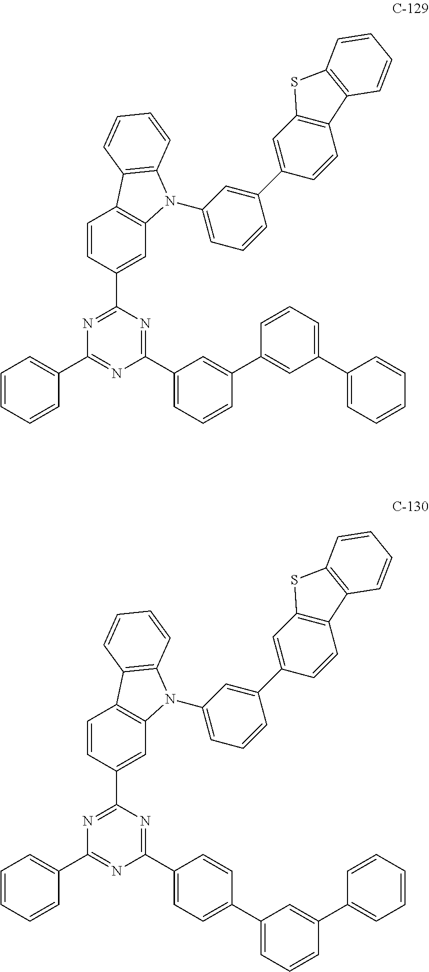 [SMILES: c1ccc(-c2cccc(-c3cccc(-c4nc(-c5ccccc5)nc(-c5ccc6c7ccccc7n(-c7cccc(-c8ccc9c(c8)sc8ccccc89)c7)c6c5)n4)c3)c2)cc1 ; c1ccc(-c2cccc(-c3ccc(-c4nc(-c5ccccc5)nc(-c5ccc6c7ccccc7n(-c7cccc(-c8ccc9c(c8)sc8ccccc89)c7)c6c5)n4)cc3)c2)cc1]

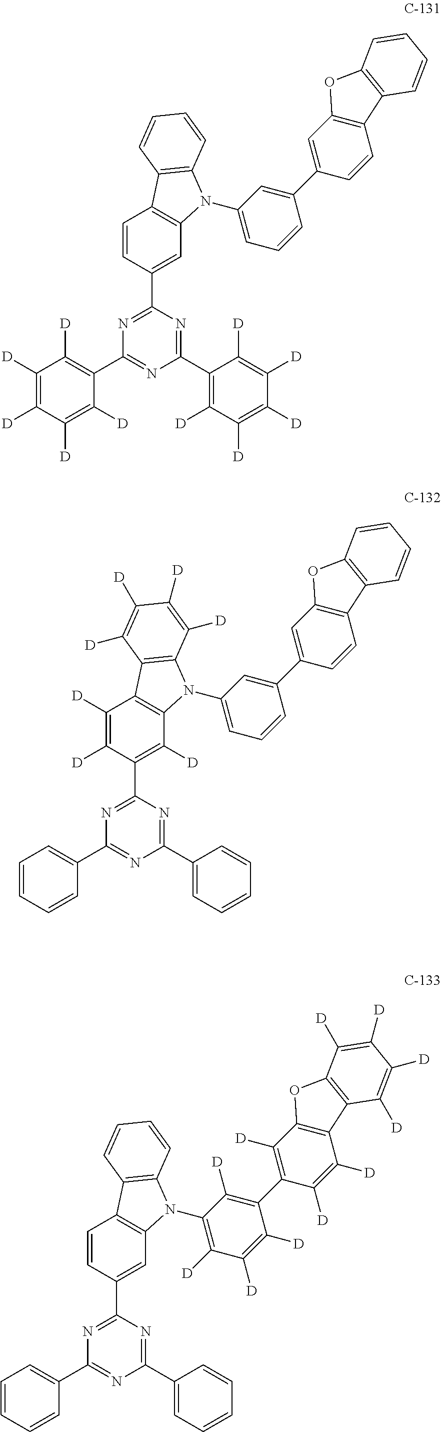 [SMILES: [2H]c1c([2H])c([2H])c(-c2nc(-c3ccc4c5ccccc5n(-c5cccc(-c6ccc7c(c6)oc6ccccc67)c5)c4c3)nc(-c3c([2H])c([2H])c([2H])c([2H])c3[2H])n2)c([2H])c1[2H] ; [2H]c1c([2H])c([2H])c2c(c1[2H])c1c([2H])c([2H])c(-c3nc(-c4ccccc4)nc(-c4ccccc4)n3)c([2H])c1n2-c1cccc(-c2ccc3c(c2)oc2ccccc23)c1 ; [2H]c1c([2H])c(-c2c([2H])c([2H])c3c(oc4c([2H])c([2H])c([2H])c([2H])c43)c2[2H])c([2H])c(-n2c3ccccc3c3ccc(-c4nc(-c5ccccc5)nc(-c5ccccc5)n4)cc32)c1[2H]]

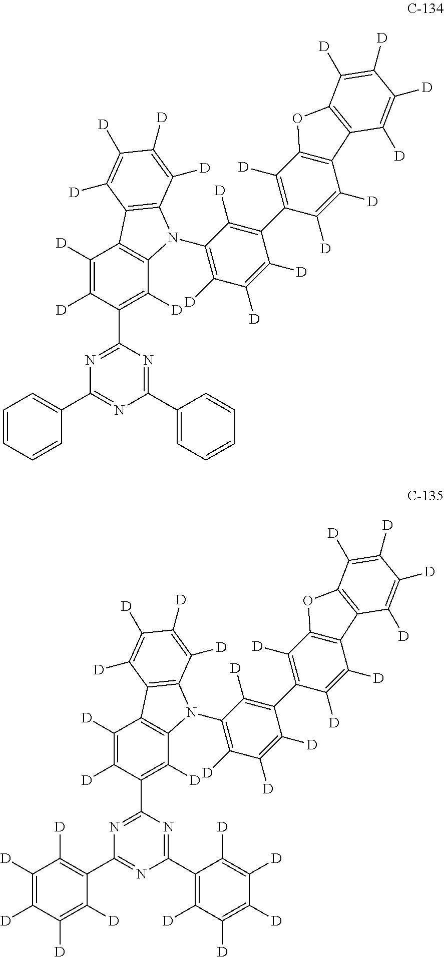 [SMILES: [2H]c1c([2H])c(-c2c([2H])c([2H])c3c(oc4c([2H])c([2H])c([2H])c([2H])c43)c2[2H])c([2H])c(-n2c3c([2H])c([2H])c([2H])c([2H])c3c3c([2H])c([2H])c(-c4nc(-c5ccccc5)nc(-c5ccccc5)n4)c([2H])c32)c1[2H] ; [2H]c1c([2H])c([2H])c(-c2nc(-c3c([2H])c([2H])c([2H])c([2H])c3[2H])nc(-c3c([2H])c([2H])c4c5c([2H])c([2H])c([2H])c([2H])c5n(-c5c([2H])c([2H])c([2H])c(-c6c([2H])c([2H])c7c(oc8c([2H])c([2H])c([2H])c([2H])c87)c6[2H])c5[2H])c4c3[2H])n2)c([2H])c1[2H]]

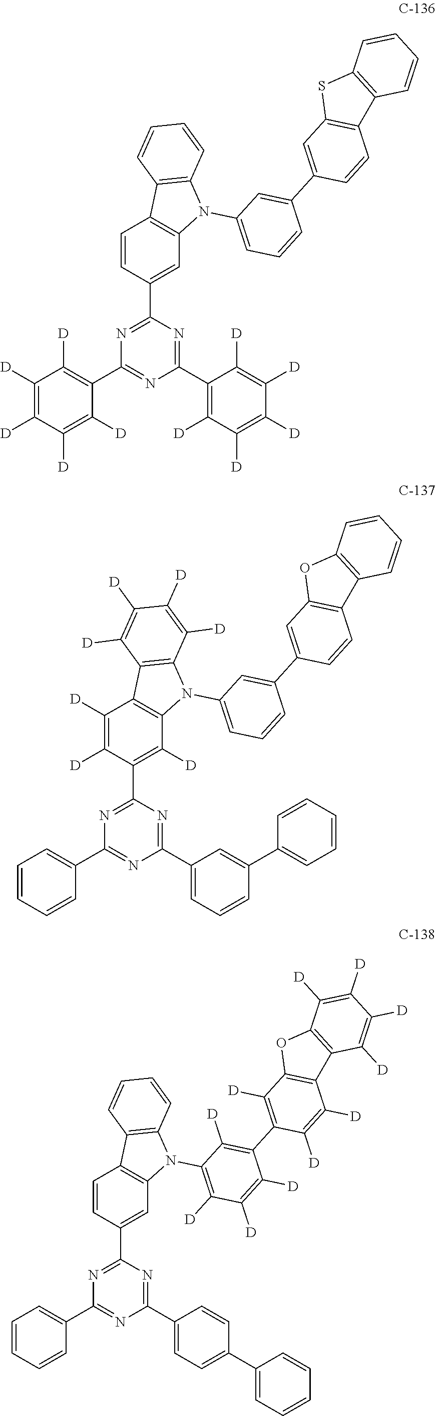 [SMILES: [2H]c1c([2H])c([2H])c(-c2nc(-c3ccc4c5ccccc5n(-c5cccc(-c6ccc7c(c6)sc6ccccc67)c5)c4c3)nc(-c3c([2H])c([2H])c([2H])c([2H])c3[2H])n2)c([2H])c1[2H] ; [2H]c1c([2H])c([2H])c2c(c1[2H])c1c([2H])c([2H])c(-c3nc(-c4ccccc4)nc(-c4cccc(-c5ccccc5)c4)n3)c([2H])c1n2-c1cccc(-c2ccc3c(c2)oc2ccccc23)c1 ; [2H]c1c([2H])c(-c2c([2H])c([2H])c3c(oc4c([2H])c([2H])c([2H])c([2H])c43)c2[2H])c([2H])c(-n2c3ccccc3c3ccc(-c4nc(-c5ccccc5)nc(-c5ccc(-c6ccccc6)cc5)n4)cc32)c1[2H]]

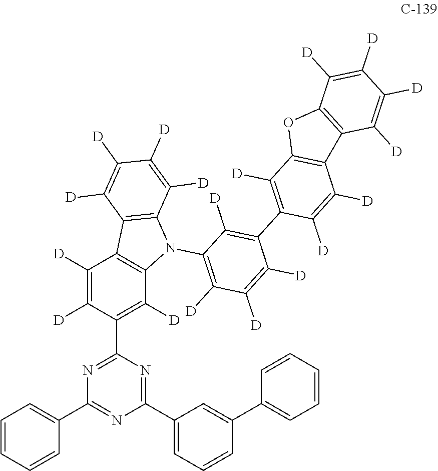 [SMILES: [2H]c1c([2H])c(-c2c([2H])c([2H])c3c(oc4c([2H])c([2H])c([2H])c([2H])c43)c2[2H])c([2H])c(-n2c3c([2H])c([2H])c([2H])c([2H])c3c3c([2H])c([2H])c(-c4nc(-c5ccccc5)nc(-c5cccc(-c6ccccc6)c5)n4)c([2H])c32)c1[2H]]

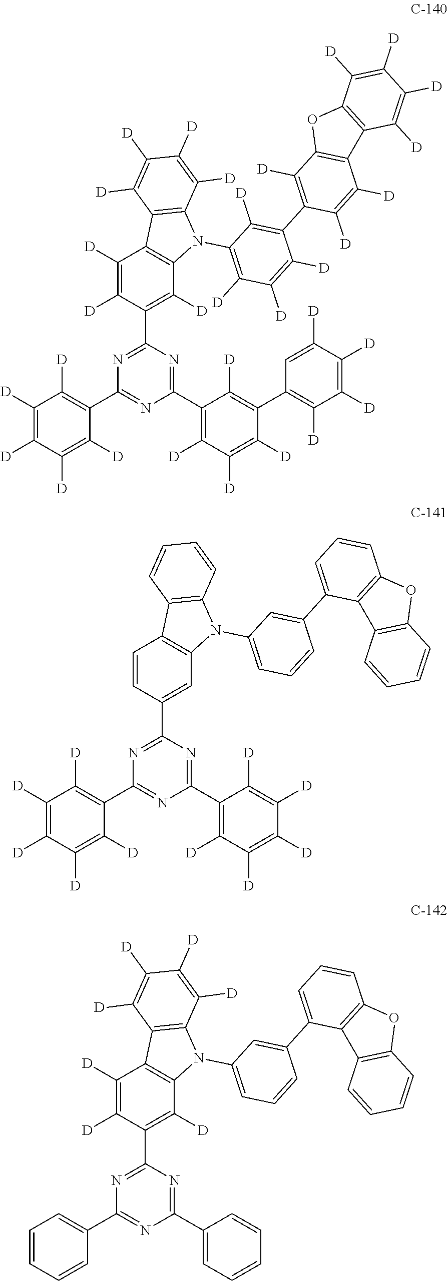

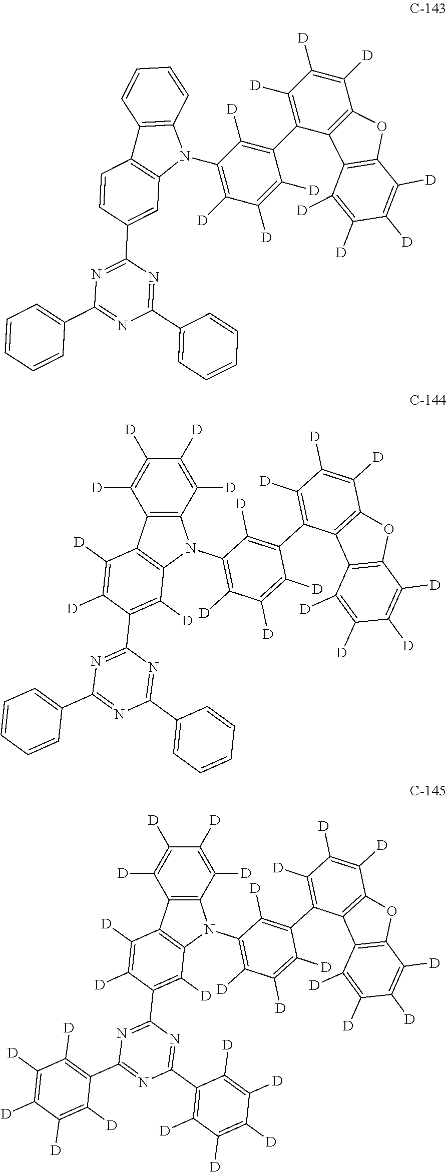

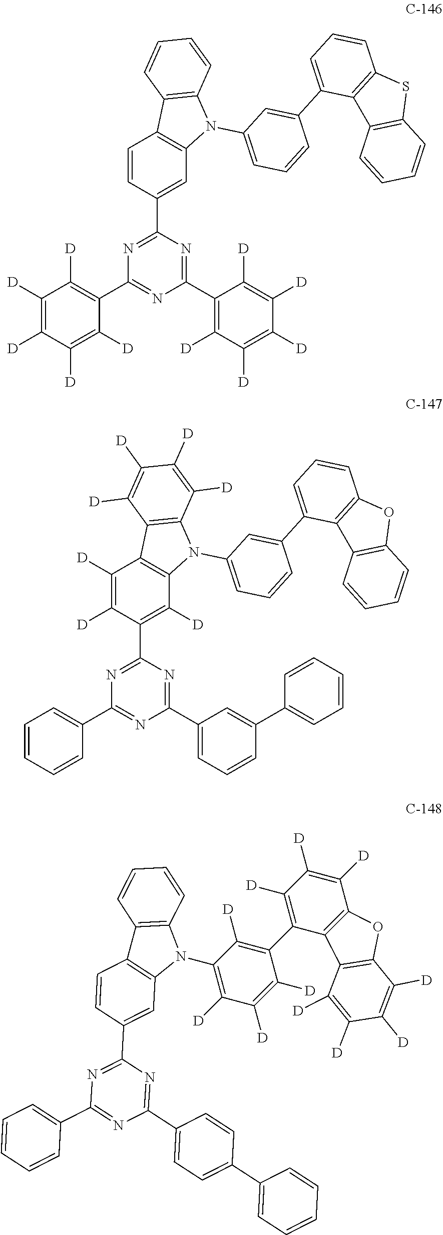 [SMILES: [2H]c1c([2H])c([2H])c(-c2nc(-c3ccc4c5ccccc5n(-c5cccc(-c6cccc7sc8ccccc8c67)c5)c4c3)nc(-c3c([2H])c([2H])c([2H])c([2H])c3[2H])n2)c([2H])c1[2H] ; [2H]c1c([2H])c([2H])c2c(c1[2H])c1c([2H])c([2H])c(-c3nc(-c4ccccc4)nc(-c4cccc(-c5ccccc5)c4)n3)c([2H])c1n2-c1cccc(-c2cccc3oc4ccccc4c23)c1 ; [2H]c1c([2H])c(-c2c([2H])c([2H])c([2H])c3oc4c([2H])c([2H])c([2H])c([2H])c4c23)c([2H])c(-n2c3ccccc3c3ccc(-c4nc(-c5ccccc5)nc(-c5ccc(-c6ccccc6)cc5)n4)cc32)c1[2H]]

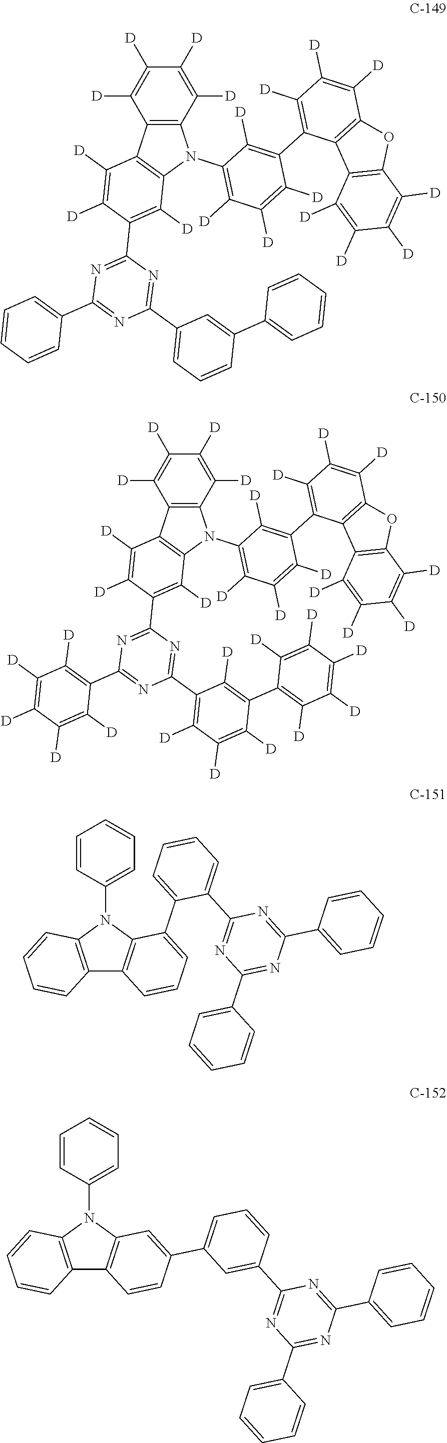

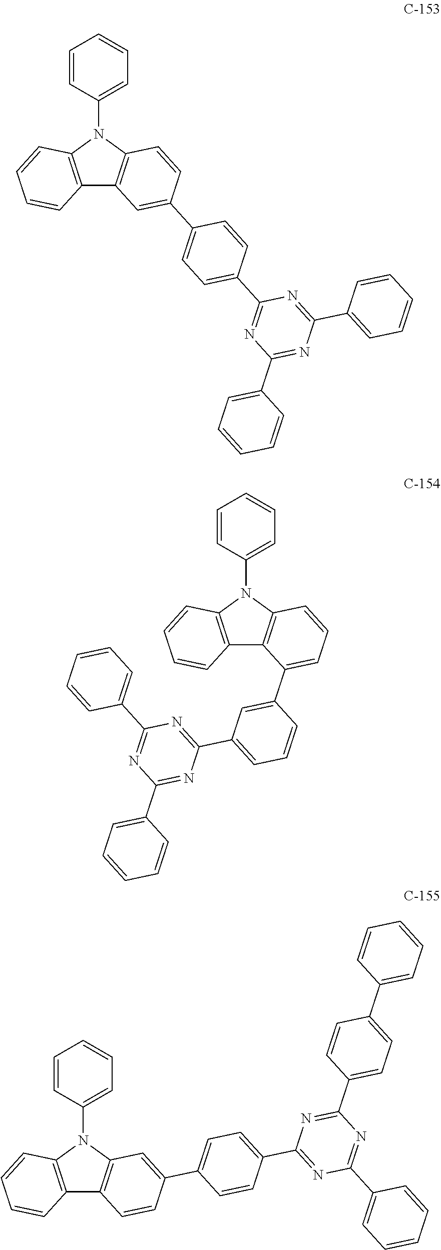

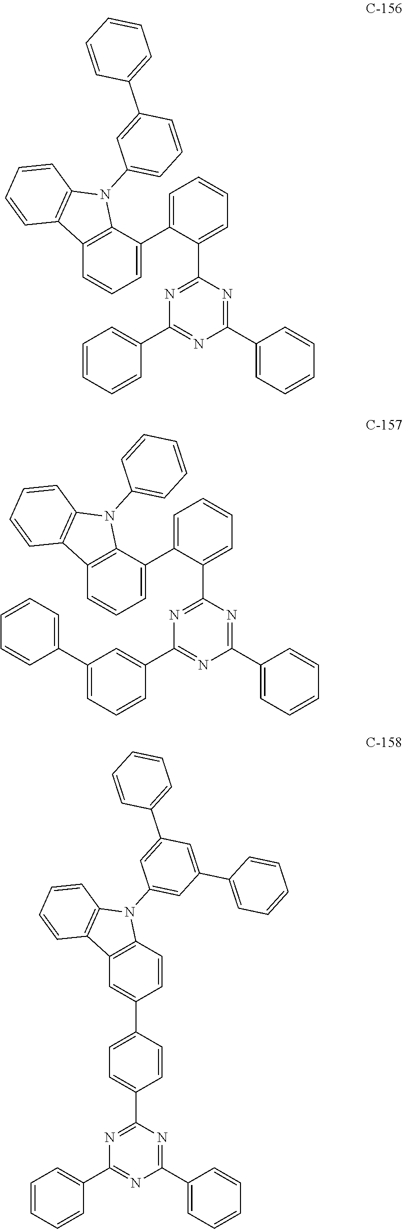

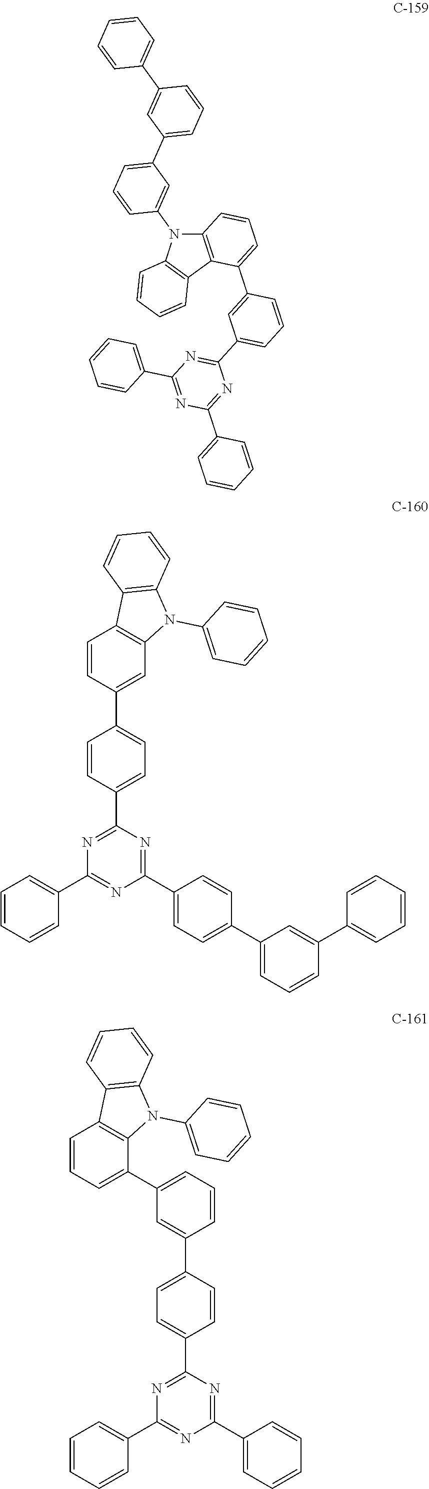 [SMILES: c1ccc(-c2cccc(-c3cccc(-n4c5ccccc5c5c(-c6cccc(-c7nc(-c8ccccc8)nc(-c8ccccc8)n7)c6)cccc54)c3)c2)cc1 ; c1ccc(-c2cccc(-c3ccc(-c4nc(-c5ccccc5)nc(-c5ccc(-c6ccc7c8ccccc8n(-c8ccccc8)c7c6)cc5)n4)cc3)c2)cc1 ; c1ccc(-c2nc(-c3ccccc3)nc(-c3ccc(-c4cccc(-c5cccc6c7ccccc7n(-c7ccccc7)c56)c4)cc3)n2)cc1]

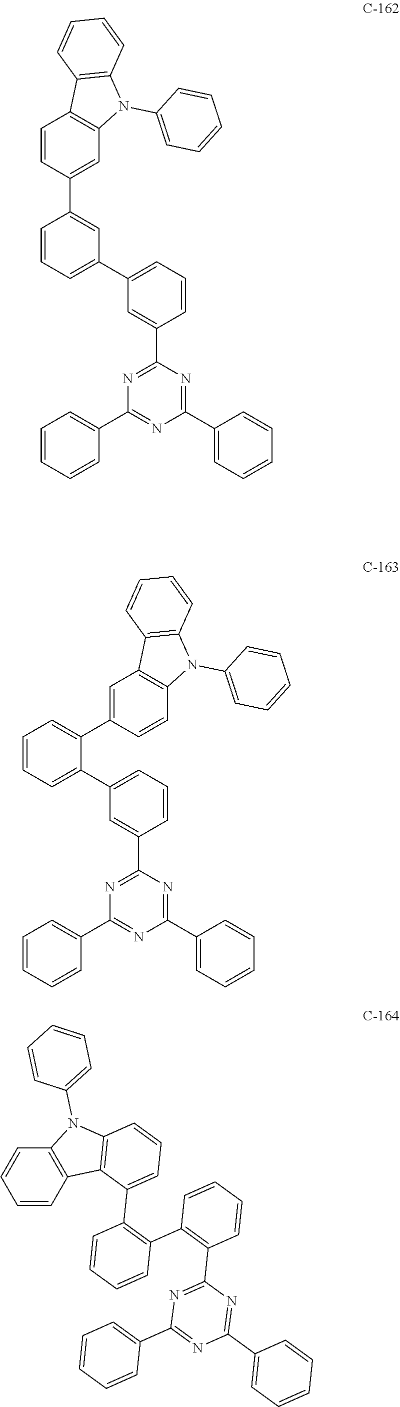

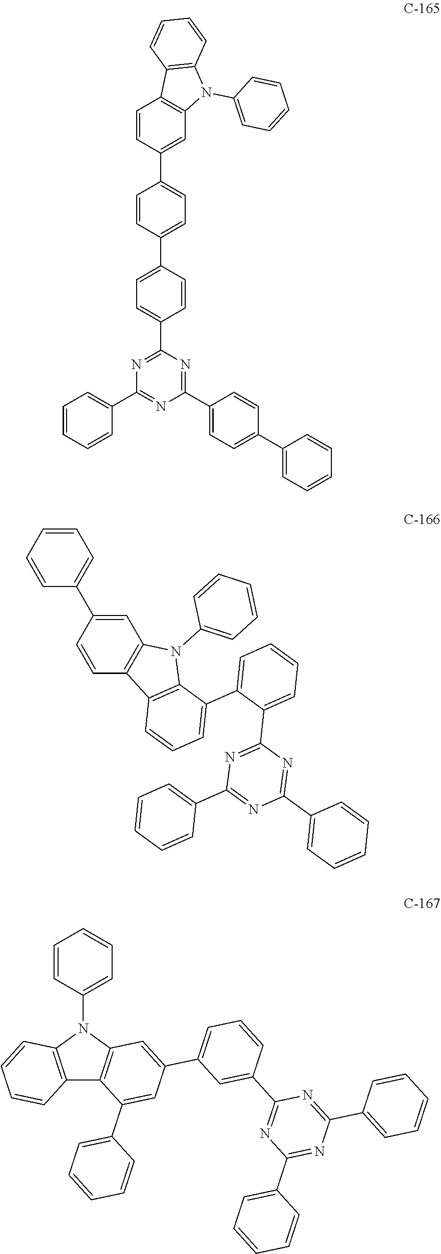 [SMILES: c1ccc(-c2ccc(-c3nc(-c4ccccc4)nc(-c4ccc(-c5ccc(-c6ccc7c8ccccc8n(-c8ccccc8)c7c6)cc5)cc4)n3)cc2)cc1 ; c1ccc(-c2ccc3c4cccc(-c5ccccc5-c5nc(-c6ccccc6)nc(-c6ccccc6)n5)c4n(-c4ccccc4)c3c2)cc1 ; c1ccc(-c2nc(-c3ccccc3)nc(-c3cccc(-c4cc(-c5ccccc5)c5c6ccccc6n(-c6ccccc6)c5c4)c3)n2)cc1]

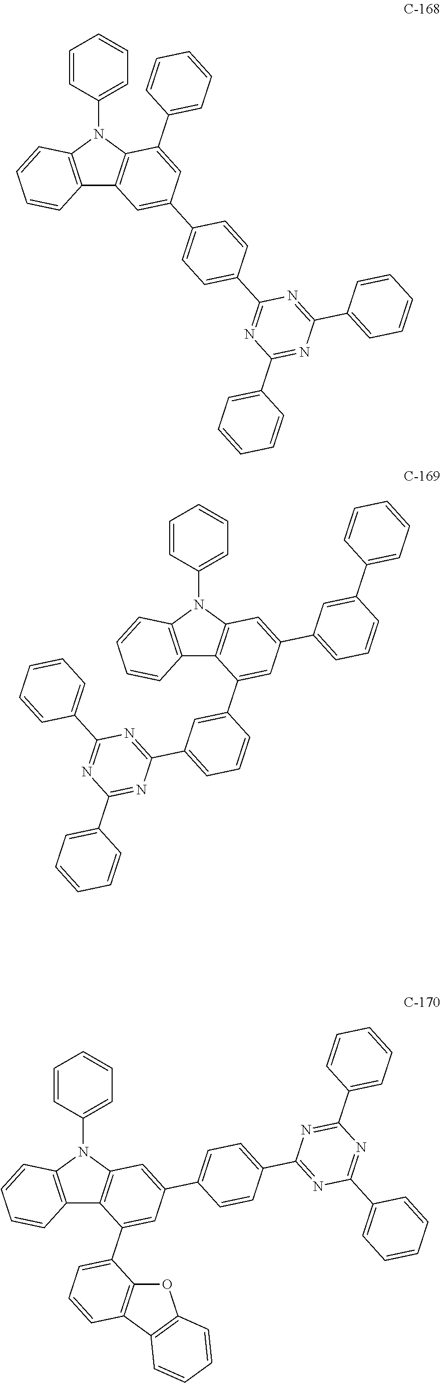 [SMILES: c1ccc(-c2nc(-c3ccccc3)nc(-c3ccc(-c4cc(-c5ccccc5)c5c(c4)c4ccccc4n5-c4ccccc4)cc3)n2)cc1 ; c1ccc(-c2cccc(-c3cc(-c4cccc(-c5nc(-c6ccccc6)nc(-c6ccccc6)n5)c4)c4c5ccccc5n(-c5ccccc5)c4c3)c2)cc1 ; c1ccc(-c2nc(-c3ccccc3)nc(-c3ccc(-c4cc(-c5cccc6c5oc5ccccc56)c5c6ccccc6n(-c6ccccc6)c5c4)cc3)n2)cc1]

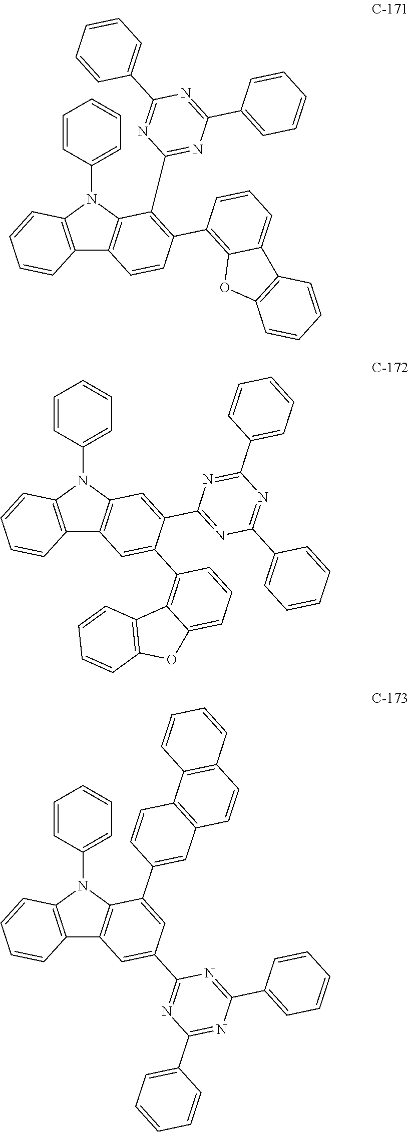

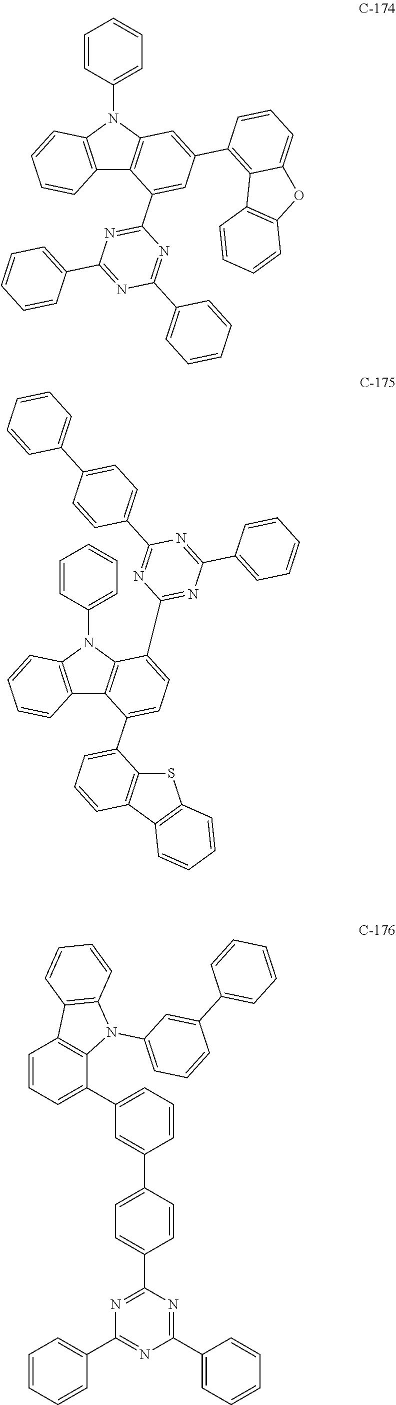 [SMILES: c1ccc(-c2nc(-c3ccccc3)nc(-c3cc(-c4cccc5oc6ccccc6c45)cc4c3c3ccccc3n4-c3ccccc3)n2)cc1 ; c1ccc(-c2ccc(-c3nc(-c4ccccc4)nc(-c4ccc(-c5cccc6c5sc5ccccc56)c5c6ccccc6n(-c6ccccc6)c45)n3)cc2)cc1 ; c1ccc(-c2cccc(-n3c4ccccc4c4cccc(-c5cccc(-c6ccc(-c7nc(-c8ccccc8)nc(-c8ccccc8)n7)cc6)c5)c43)c2)cc1]

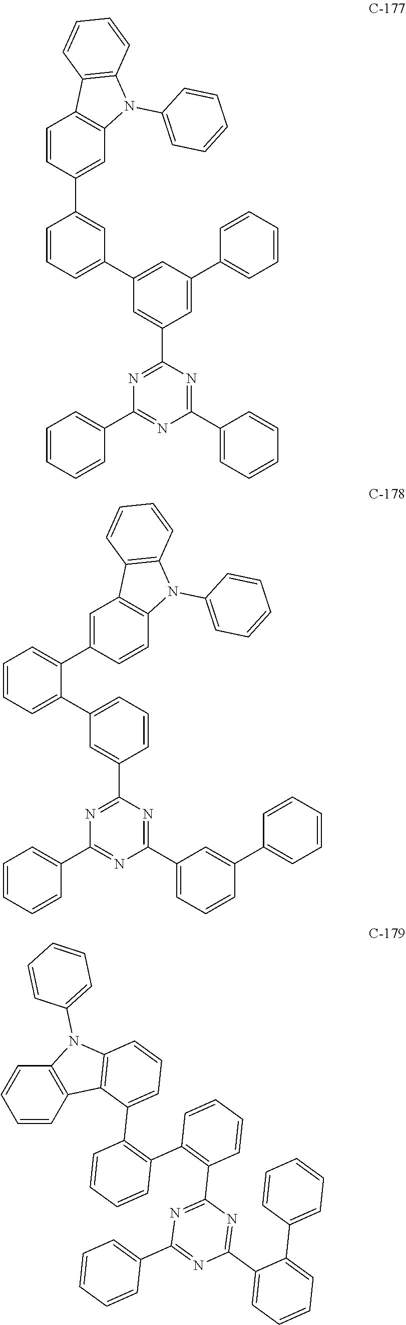

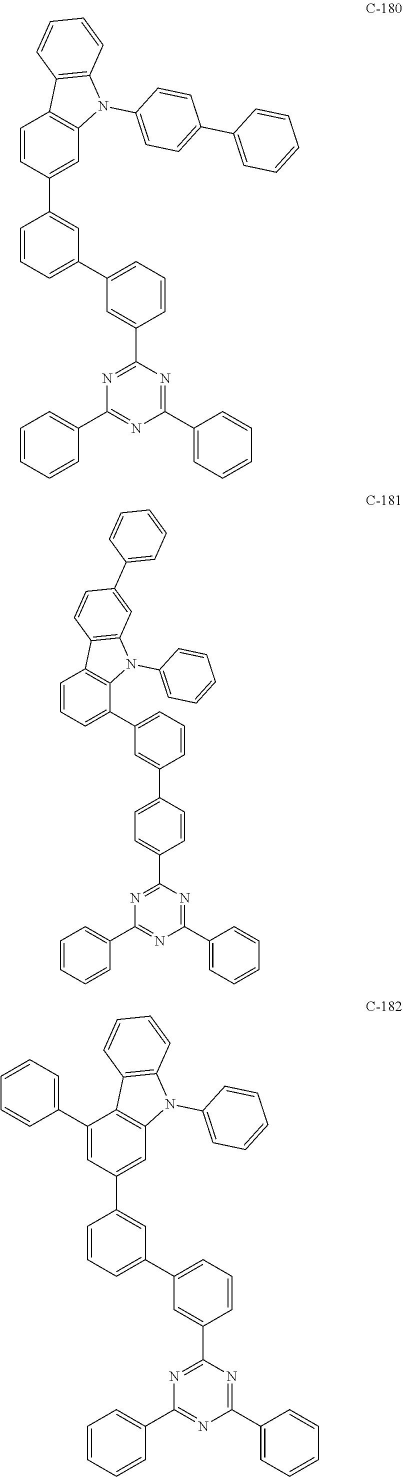 [SMILES: c1ccc(-c2ccc(-n3c4ccccc4c4ccc(-c5cccc(-c6cccc(-c7nc(-c8ccccc8)nc(-c8ccccc8)n7)c6)c5)cc43)cc2)cc1 ; c1ccc(-c2ccc3c4cccc(-c5cccc(-c6ccc(-c7nc(-c8ccccc8)nc(-c8ccccc8)n7)cc6)c5)c4n(-c4ccccc4)c3c2)cc1 ; c1ccc(-c2nc(-c3ccccc3)nc(-c3cccc(-c4cccc(-c5cc(-c6ccccc6)c6c7ccccc7n(-c7ccccc7)c6c5)c4)c3)n2)cc1]

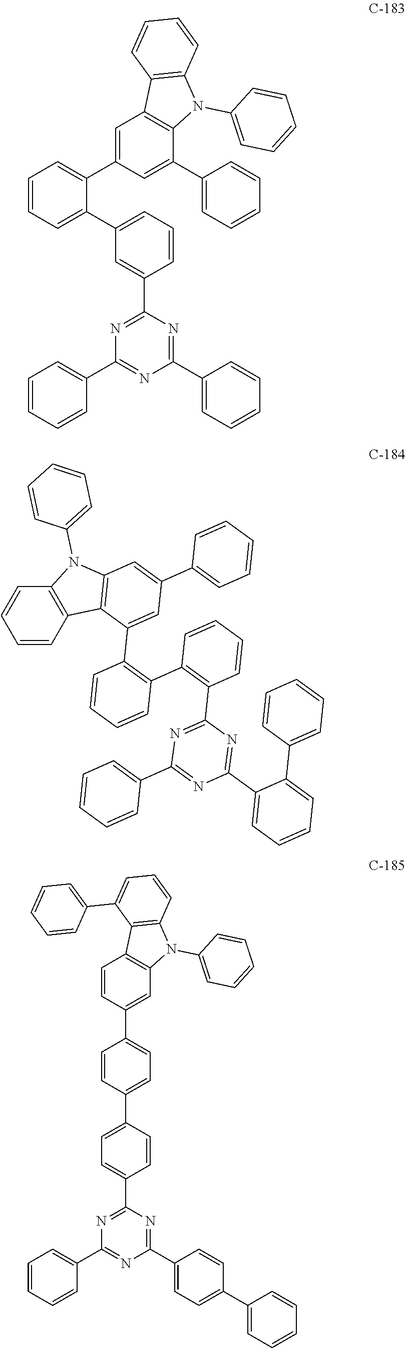

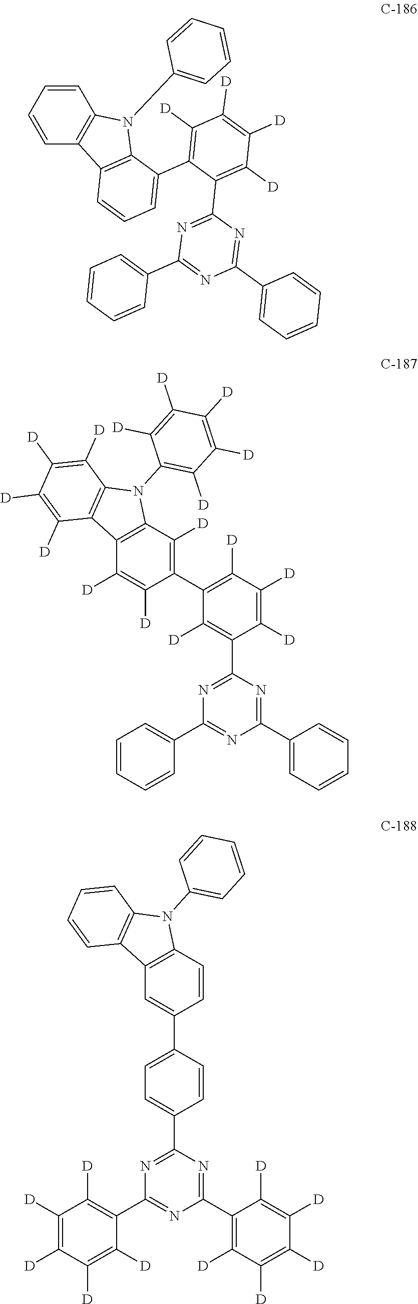 [SMILES: [2H]c1c([2H])c([2H])c(-c2cccc3c4ccccc4n(-c4ccccc4)c23)c(-c2nc(-c3ccccc3)nc(-c3ccccc3)n2)c1[2H] ; [2H]c1c([2H])c([2H])c(-n2c3c([2H])c([2H])c([2H])c([2H])c3c3c([2H])c([2H])c(-c4c([2H])c([2H])c([2H])c(-c5nc(-c6ccccc6)nc(-c6ccccc6)n5)c4[2H])c([2H])c32)c([2H])c1[2H] ; [2H]c1c([2H])c([2H])c(-c2nc(-c3ccc(-c4ccc5c(c4)c4ccccc4n5-c4ccccc4)cc3)nc(-c3c([2H])c([2H])c([2H])c([2H])c3[2H])n2)c([2H])c1[2H]]

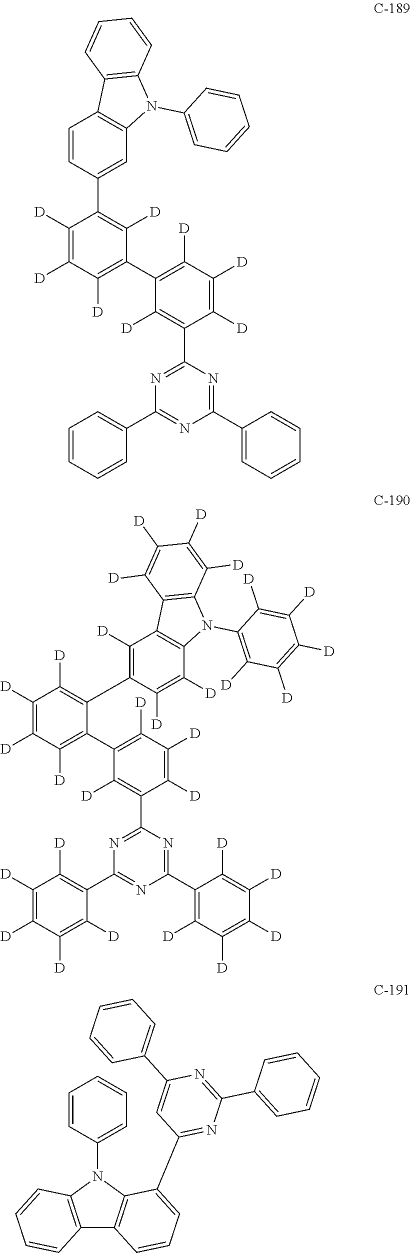 [SMILES: [2H]c1c([2H])c(-c2ccc3c4ccccc4n(-c4ccccc4)c3c2)c([2H])c(-c2c([2H])c([2H])c([2H])c(-c3nc(-c4ccccc4)nc(-c4ccccc4)n3)c2[2H])c1[2H] ; [2H]c1c([2H])c([2H])c(-c2nc(-c3c([2H])c([2H])c([2H])c([2H])c3[2H])nc(-c3c([2H])c([2H])c([2H])c(-c4c([2H])c([2H])c([2H])c([2H])c4-c4c([2H])c([2H])c5c(c4[2H])c4c([2H])c([2H])c([2H])c([2H])c4n5-c4c([2H])c([2H])c([2H])c([2H])c4[2H])c3[2H])n2)c([2H])c1[2H] ; c1ccc(-c2cc(-c3cccc4c5ccccc5n(-c5ccccc5)c34)nc(-c3ccccc3)n2)cc1]

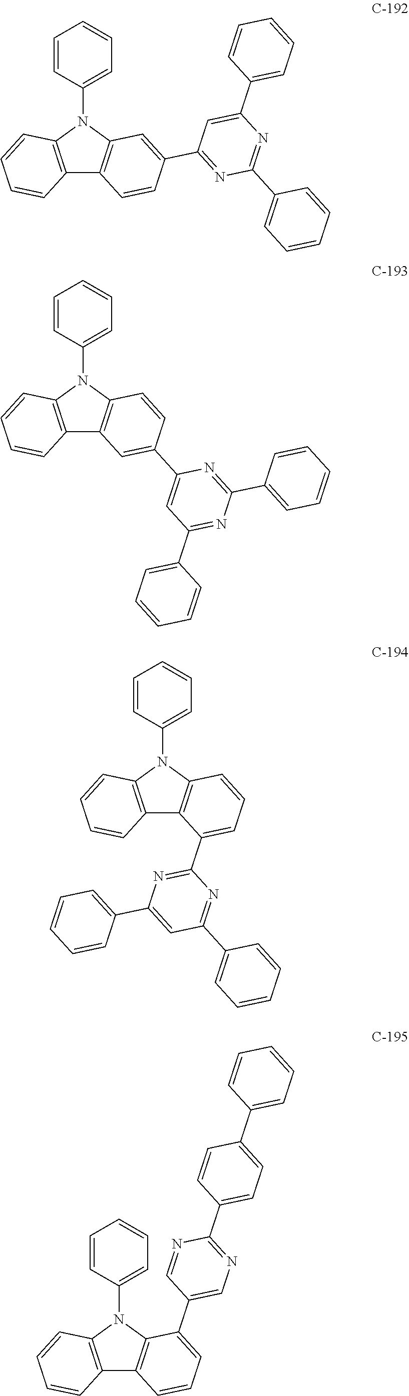

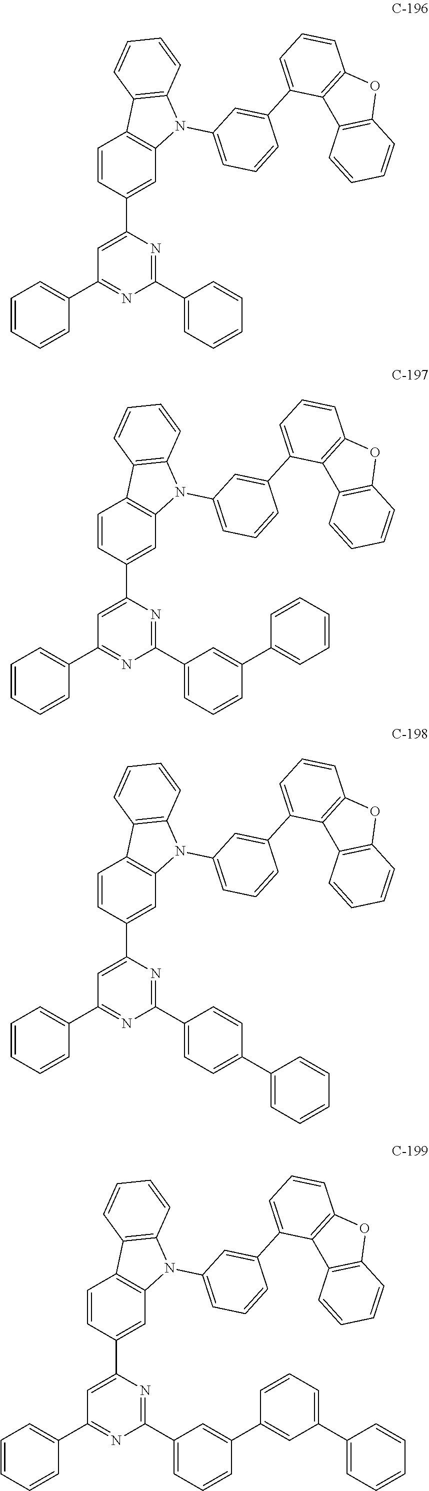 [SMILES: c1ccc(-c2cc(-c3ccc4c5ccccc5n(-c5cccc(-c6cccc7oc8ccccc8c67)c5)c4c3)nc(-c3ccccc3)n2)cc1 ; c1ccc(-c2cccc(-c3nc(-c4ccccc4)cc(-c4ccc5c6ccccc6n(-c6cccc(-c7cccc8oc9ccccc9c78)c6)c5c4)n3)c2)cc1 ; c1ccc(-c2ccc(-c3nc(-c4ccccc4)cc(-c4ccc5c6ccccc6n(-c6cccc(-c7cccc8oc9ccccc9c78)c6)c5c4)n3)cc2)cc1 ; c1ccc(-c2cccc(-c3cccc(-c4nc(-c5ccccc5)cc(-c5ccc6c7ccccc7n(-c7cccc(-c8cccc9oc%10ccccc%10c89)c7)c6c5)n4)c3)c2)cc1]

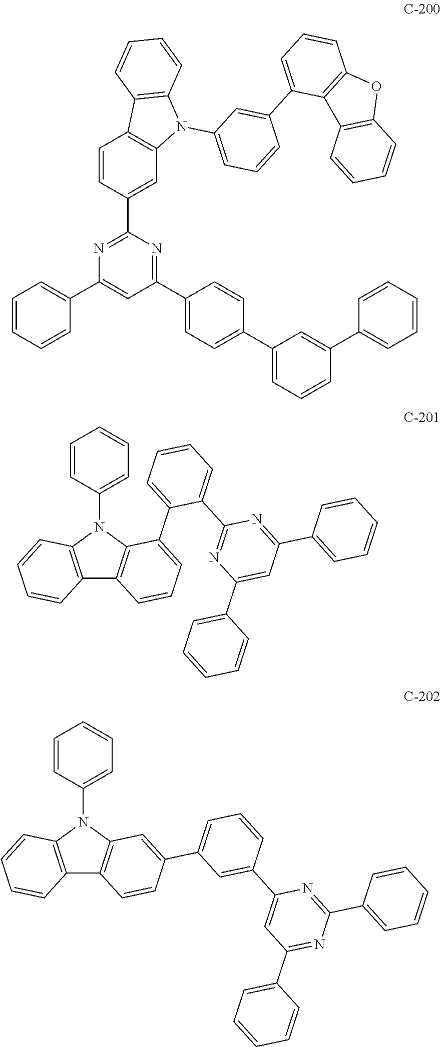 [SMILES: c1ccc(-c2cccc(-c3ccc(-c4cc(-c5ccccc5)nc(-c5ccc6c7ccccc7n(-c7cccc(-c8cccc9oc%10ccccc%10c89)c7)c6c5)n4)cc3)c2)cc1 ; c1ccc(-c2cc(-c3ccccc3)nc(-c3ccccc3-c3cccc4c5ccccc5n(-c5ccccc5)c34)n2)cc1 ; c1ccc(-c2cc(-c3cccc(-c4ccc5c6ccccc6n(-c6ccccc6)c5c4)c3)nc(-c3ccccc3)n2)cc1]

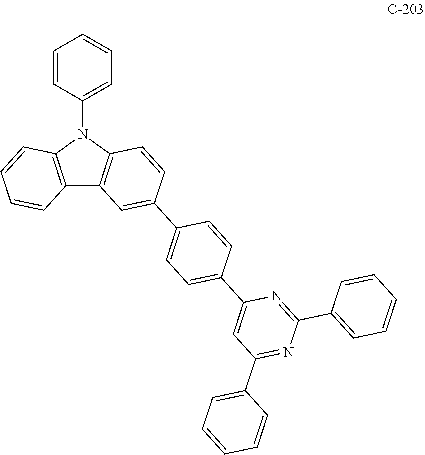 [SMILES: c1ccc(-c2cc(-c3ccc(-c4ccc5c(c4)c4ccccc4n5-c4ccccc4)cc3)nc(-c3ccccc3)n2)cc1]

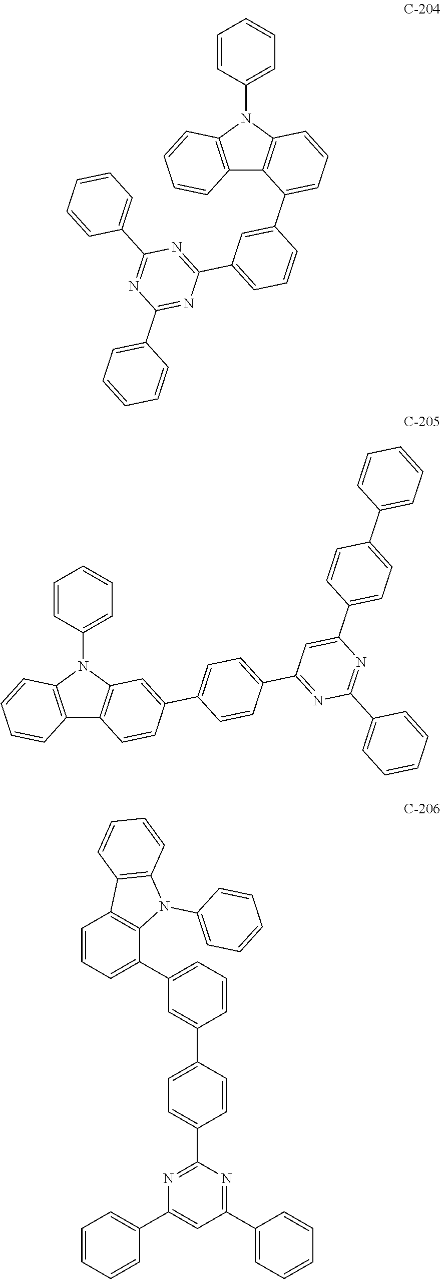 [SMILES: c1ccc(-c2nc(-c3ccccc3)nc(-c3cccc(-c4cccc5c4c4ccccc4n5-c4ccccc4)c3)n2)cc1 ; c1ccc(-c2ccc(-c3cc(-c4ccc(-c5ccc6c7ccccc7n(-c7ccccc7)c6c5)cc4)nc(-c4ccccc4)n3)cc2)cc1 ; c1ccc(-c2cc(-c3ccccc3)nc(-c3ccc(-c4cccc(-c5cccc6c7ccccc7n(-c7ccccc7)c56)c4)cc3)n2)cc1]

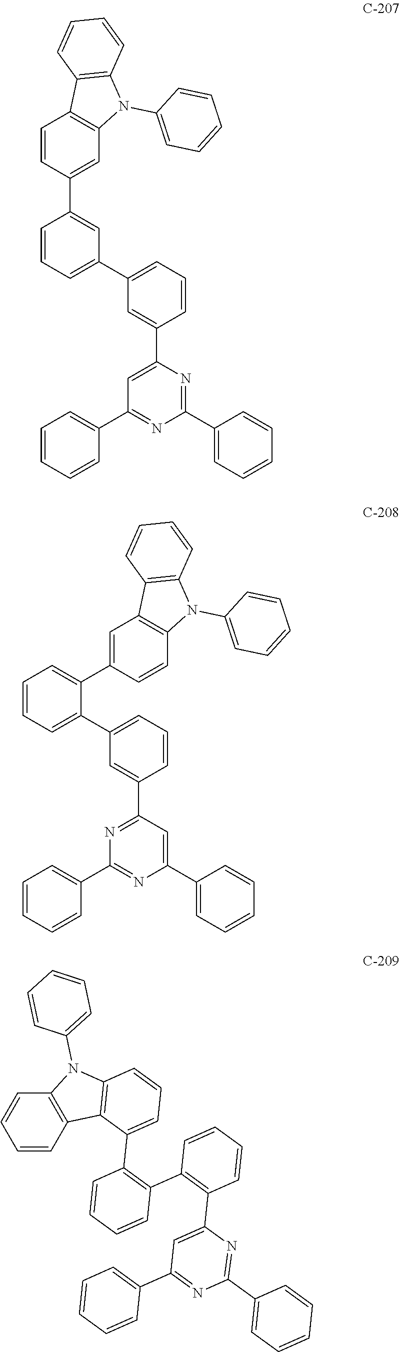

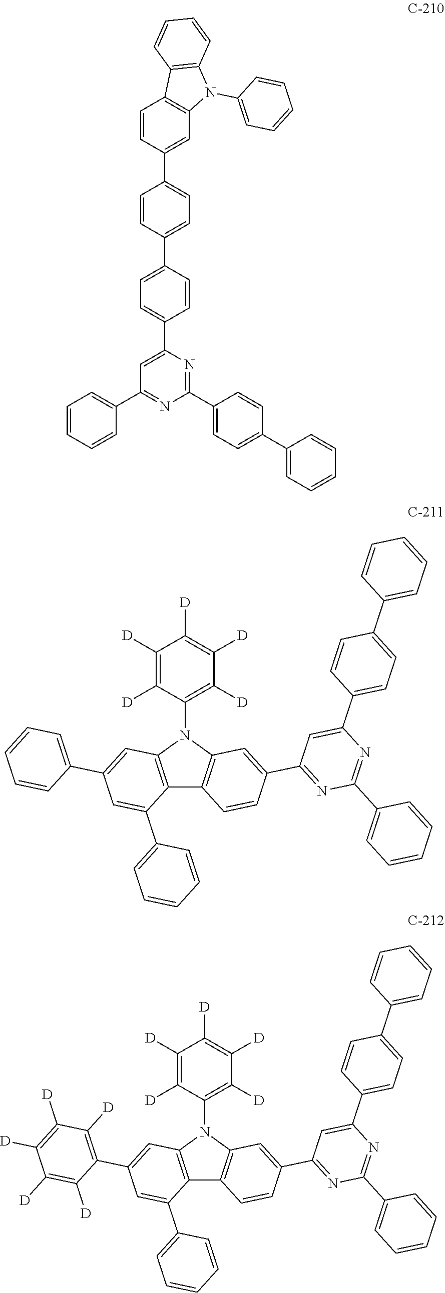 [SMILES: c1ccc(-c2ccc(-c3nc(-c4ccccc4)cc(-c4ccc(-c5ccc(-c6ccc7c8ccccc8n(-c8ccccc8)c7c6)cc5)cc4)n3)cc2)cc1 ; [2H]c1c([2H])c([2H])c(-n2c3cc(-c4cc(-c5ccc(-c6ccccc6)cc5)nc(-c5ccccc5)n4)ccc3c3c(-c4ccccc4)cc(-c4ccccc4)cc32)c([2H])c1[2H] ; [2H]c1c([2H])c([2H])c(-c2cc(-c3ccccc3)c3c4ccc(-c5cc(-c6ccc(-c7ccccc7)cc6)nc(-c6ccccc6)n5)cc4n(-c4c([2H])c([2H])c([2H])c([2H])c4[2H])c3c2)c([2H])c1[2H]]

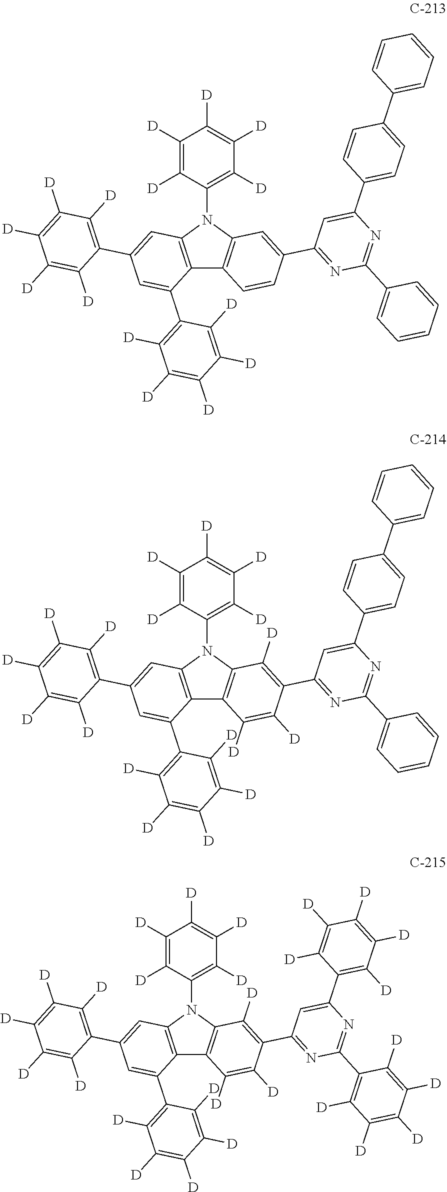

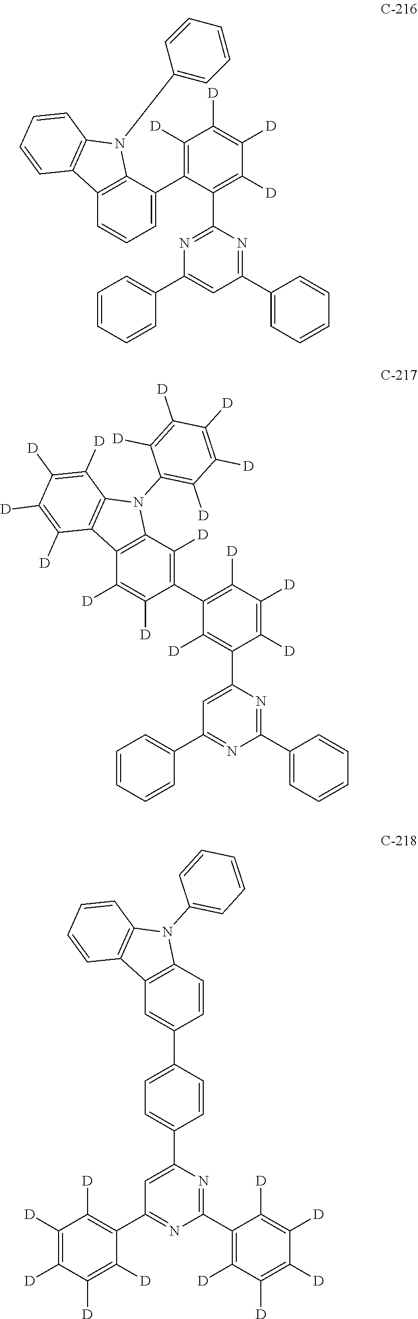

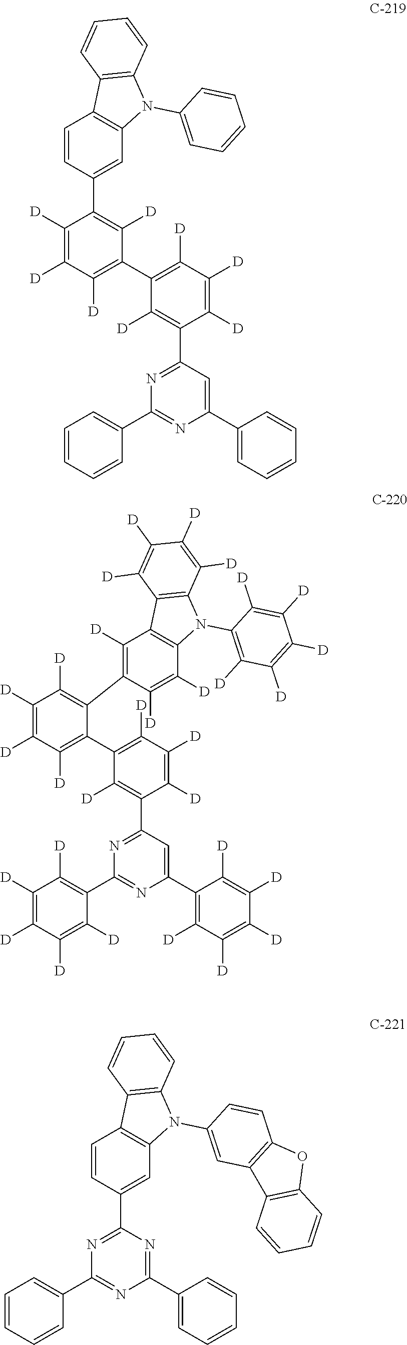 [SMILES: [2H]c1c([2H])c(-c2ccc3c4ccccc4n(-c4ccccc4)c3c2)c([2H])c(-c2c([2H])c([2H])c([2H])c(-c3cc(-c4ccccc4)nc(-c4ccccc4)n3)c2[2H])c1[2H] ; [2H]c1c([2H])c([2H])c(-c2cc(-c3c([2H])c([2H])c([2H])c(-c4c([2H])c([2H])c([2H])c([2H])c4-c4c([2H])c([2H])c5c(c4[2H])c4c([2H])c([2H])c([2H])c([2H])c4n5-c4c([2H])c([2H])c([2H])c([2H])c4[2H])c3[2H])nc(-c3c([2H])c([2H])c([2H])c([2H])c3[2H])n2)c([2H])c1[2H] ; c1ccc(-c2nc(-c3ccccc3)nc(-c3ccc4c5ccccc5n(-c5ccc6oc7ccccc7c6c5)c4c3)n2)cc1]

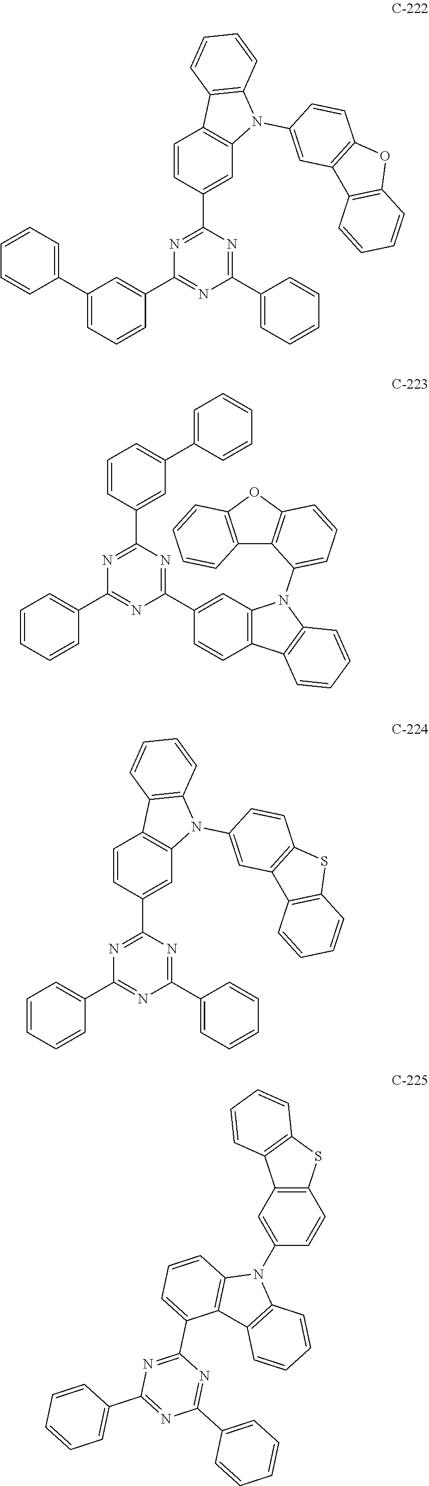 [SMILES: c1ccc(-c2cccc(-c3nc(-c4ccccc4)nc(-c4ccc5c6ccccc6n(-c6ccc7oc8ccccc8c7c6)c5c4)n3)c2)cc1 ; c1ccc(-c2cccc(-c3nc(-c4ccccc4)nc(-c4ccc5c6ccccc6n(-c6cccc7oc8ccccc8c67)c5c4)n3)c2)cc1 ; c1ccc(-c2nc(-c3ccccc3)nc(-c3ccc4c5ccccc5n(-c5ccc6sc7ccccc7c6c5)c4c3)n2)cc1 ; c1ccc(-c2nc(-c3ccccc3)nc(-c3cccc4c3c3ccccc3n4-c3ccc4sc5ccccc5c4c3)n2)cc1]

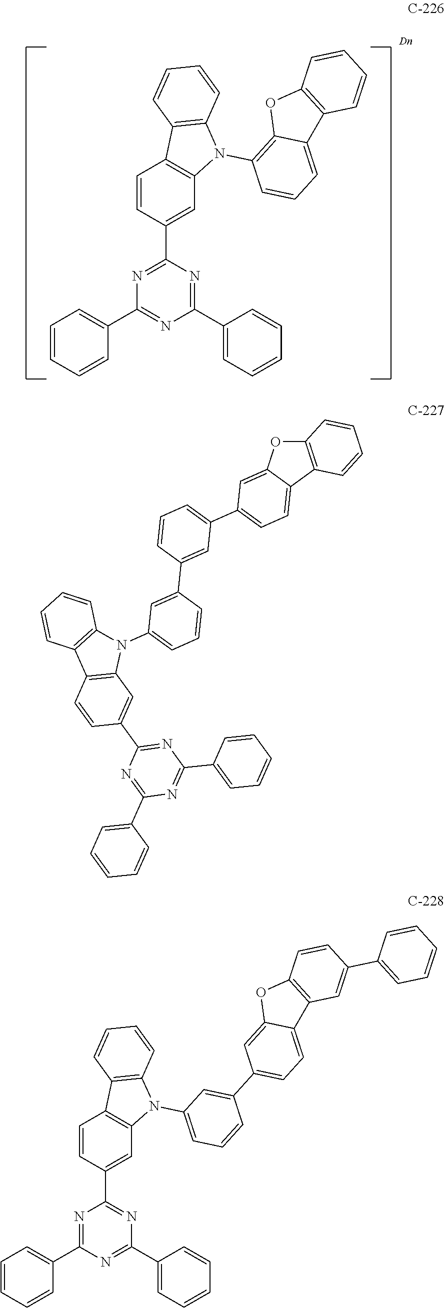

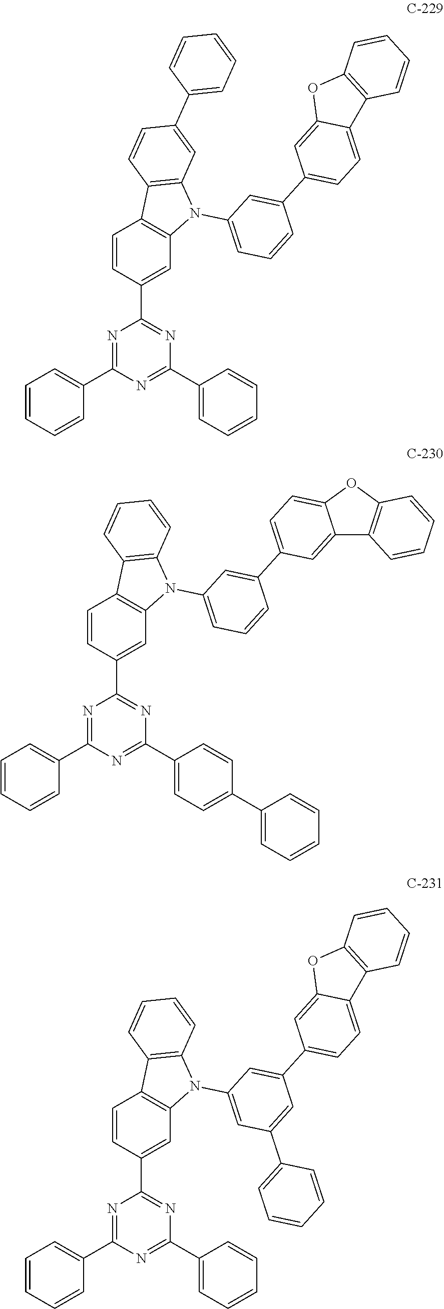 [SMILES: c1ccc(-c2ccc3c4ccc(-c5nc(-c6ccccc6)nc(-c6ccccc6)n5)cc4n(-c4cccc(-c5ccc6c(c5)oc5ccccc56)c4)c3c2)cc1 ; c1ccc(-c2ccc(-c3nc(-c4ccccc4)nc(-c4ccc5c6ccccc6n(-c6cccc(-c7ccc8oc9ccccc9c8c7)c6)c5c4)n3)cc2)cc1 ; c1ccc(-c2cc(-c3ccc4c(c3)oc3ccccc34)cc(-n3c4ccccc4c4ccc(-c5nc(-c6ccccc6)nc(-c6ccccc6)n5)cc43)c2)cc1]

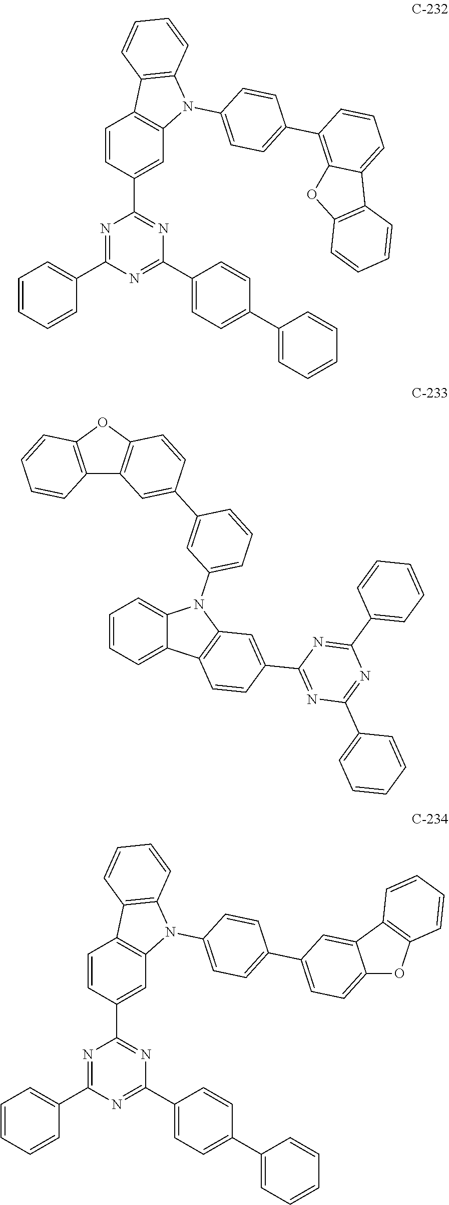 [SMILES: c1ccc(-c2ccc(-c3nc(-c4ccccc4)nc(-c4ccc5c6ccccc6n(-c6ccc(-c7cccc8c7oc7ccccc78)cc6)c5c4)n3)cc2)cc1 ; c1ccc(-c2nc(-c3ccccc3)nc(-c3ccc4c5ccccc5n(-c5cccc(-c6ccc7oc8ccccc8c7c6)c5)c4c3)n2)cc1 ; c1ccc(-c2ccc(-c3nc(-c4ccccc4)nc(-c4ccc5c6ccccc6n(-c6ccc(-c7ccc8oc9ccccc9c8c7)cc6)c5c4)n3)cc2)cc1]

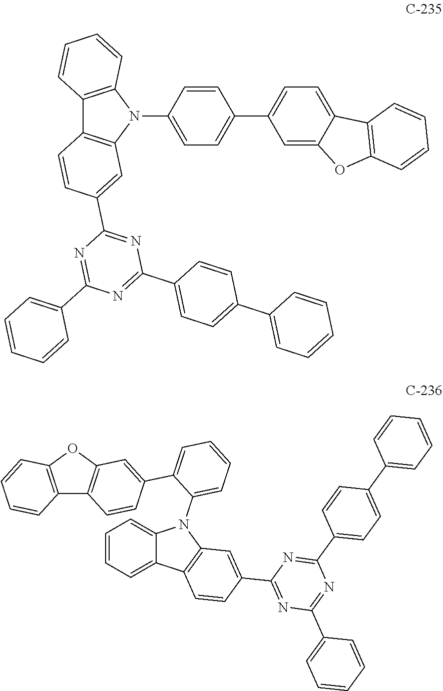 [SMILES: c1ccc(-c2ccc(-c3nc(-c4ccccc4)nc(-c4ccc5c6ccccc6n(-c6ccc(-c7ccc8c(c7)oc7ccccc78)cc6)c5c4)n3)cc2)cc1 ; c1ccc(-c2ccc(-c3nc(-c4ccccc4)nc(-c4ccc5c6ccccc6n(-c6ccccc6-c6ccc7c(c6)oc6ccccc67)c5c4)n3)cc2)cc1]

The compound represented by Formula 2 of the present disclosure will be described in more detail as follows.

In Formula 2 above, A₁ and A₂ may be each independently a substituted or unsubstituted, (C6-C30)aryl, dibenzofuranyl, dibenzothiophenyl, or carbazolyl, and preferably a substituted or unsubstituted, (C6-C25)aryl, dibenzofuranyl, dibenzothiophenyl, or carbazolyl. According to one embodiment of the present disclosure, A₁ and A₂ may be each independently phenyl unsubstituted or substituted with deuterium, biphenyl unsubstituted or substituted with deuterium, terphenyl unsubstituted or substituted with deuterium, naphthyl unsubstituted or substituted with deuterium, fluorenyl unsubstituted or substituted with at least one of deuterium, (C1-C30)alkyl, and (C6-C30)aryl, benzofluorenyl unsubstituted or substituted with at least one of deuterium, (C1-C30)alkyl, and (C6-C30)aryl, triphenylenyl unsubstituted or substituted with deuterium, fluoranthenyl unsubstituted or substituted with deuterium, phenanthrenyl unsubstituted or substituted with deuterium, dibenzofuranyl unsubstituted or substituted with deuterium, carbazolyl unsubstituted or substituted with deuterium, dibenzothiophenyl unsubstituted or substituted with deuterium, or a combination thereof. Specifically, A₁ and A₂ may be each independently, a substituted or unsubstituted, phenyl, p-biphenyl, m-biphenyl, o-biphenyl, p-terphenyl, m-terphenyl, o-terphenyl, naphthyl, fluorenyl, benzofluorenyl, triphenylenyl, fluoranthenyl, phenanthrenyl, dibenzofuranyl, dibenzothiophenyl, or carbazolyl. The substituents of the above substituents may be one or more of deuterium, (C6-C30)aryl, and (3- to 30-membered)heteroaryl, and preferably one or more of deuterium, (C6-C18)aryl, and (5- to 20-membered)heteroaryl. For example, A₁ and A₂ may be each independently phenyl unsubstituted or substituted with one or more of naphthyl, triphenylenyl, dibenzofuranyl, and dibenzothiophenyl; naphthyl unsubstituted or substituted with phenyl; p-biphenyl; m-biphenyl; o-biphenyl; o-terphenyl; m-terphenyl; p-terphenyl; triphenylenyl; dibenzofuranyl unsubstituted or substituted with phenyl; dibenzothiophenyl unsubstituted or substituted with phenyl; or carbazolyl unsubstituted or substituted with at least one of phenyl and naphthyl. They may be further substituted with deuterium.

In Formula 2 above, any one of X₁₅ to X₁₈ and any one of X₁₉ to X₂₂ are connected to each other to form a single bond. X₁₁ to X₁₄, X₂₃ to X₂₆, and X₁₅ to X₂₂, which do not form a single bond, each independently represent hydrogen, deuterium, a substituted or unsubstituted (C6-C30)aryl, or a substituted or unsubstituted (3- to 30-membered)heteroaryl; or may be linked to an adjacent substituent to form a ring(s). According to one embodiment, X₁₁ to X₁₄, X₂₃ to X₂₆, and X₁₅ to X₂₂, which do not form a single bond, may be each independently hydrogen, deuterium, a substituted or unsubstituted (C6-C30)aryl, or a substituted or unsubstituted (5- to 30-membered)heteroaryl. For example, X₁₁ to X₁₄, X₂₃ to X₂₆, and X₁₅ to X₂₂, which do not form a single bond, may be hydrogen or deuterium. In one embodiment, at least four of X₁₁ to X₂₆ may be deuterium.

According to one embodiment of the present disclosure, at least one of X₁₁, X₁₈, X₁₉ and X₂₆ may be deuterium, preferably at least two, more preferably at least three, and still more preferably may be all deuterium.

According to one embodiment of the present disclosure, in one compound represented by Formula 2, the deuterium substitution rate is from about 40% to about 100%, preferably from about 50% to about 100%, more preferably from about 60% to about 100%, and still more preferably from about 70% to about 100%. According to another embodiment of the present disclosure, Formula 2 may comprise more than one deuterium, i.e., in one compound represented by Formula 2, the deuterium substitution rate is from about 40% to less than 100%, preferably from about 50% to less than 100%, more preferably from about 60% to less than 100%, and even more preferably from about 70% to less than 100%. The upper limit of said substitution rate may be 100%, or may be less than 100%, such as about 99%. In other words, a compound of Formula 2 may be a compound in which all or part of hydrogen is substituted with deuterium.

According to one embodiment of the present disclosure, the deuterium substitution rate of X₁₁ to X₂₆ may be from about 25% to about 100%, preferably from about 35% to about 100%, more preferably from about 45% to about 100%, and even more preferably from about 55% to about 100%. According to another embodiment of the present disclosure, one or more of X₁₁ to X₂₆ may be hydrogen, i.e., the deuterium substitution rate of X₁₁ to X₂₆ may be from about 25% to less than 100%, preferably from about 35% to less than 100%, more preferably from about 45% to less than 100%, even more preferably from about 55% to less than 100%. The upper limit of said substitution rate may be 100%, or may be less than 100%, for example, may be about 99%.

According to one embodiment of the present disclosure, the Formula 2 may be represented by any one of the following formulas 2-1 to 2-8.

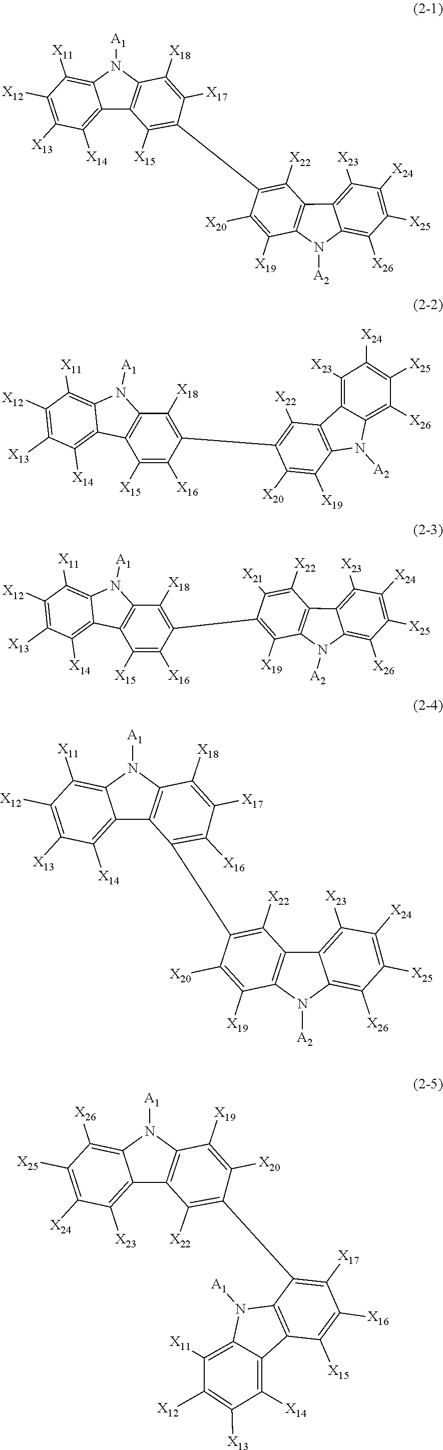

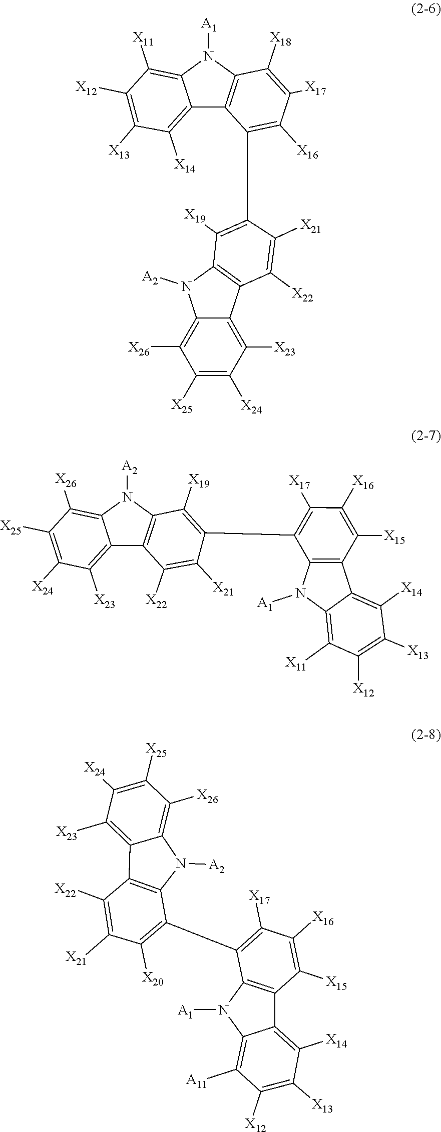

In formulas 2-1 to 2-8 above, A₁, A₂, and X₁₁ to X₂₆ are as defined in Formula 2 above.

According to one embodiment of the present disclosure, the compound represented by Formula 2 may be one or more selected from the following compounds, but is not limited thereto.

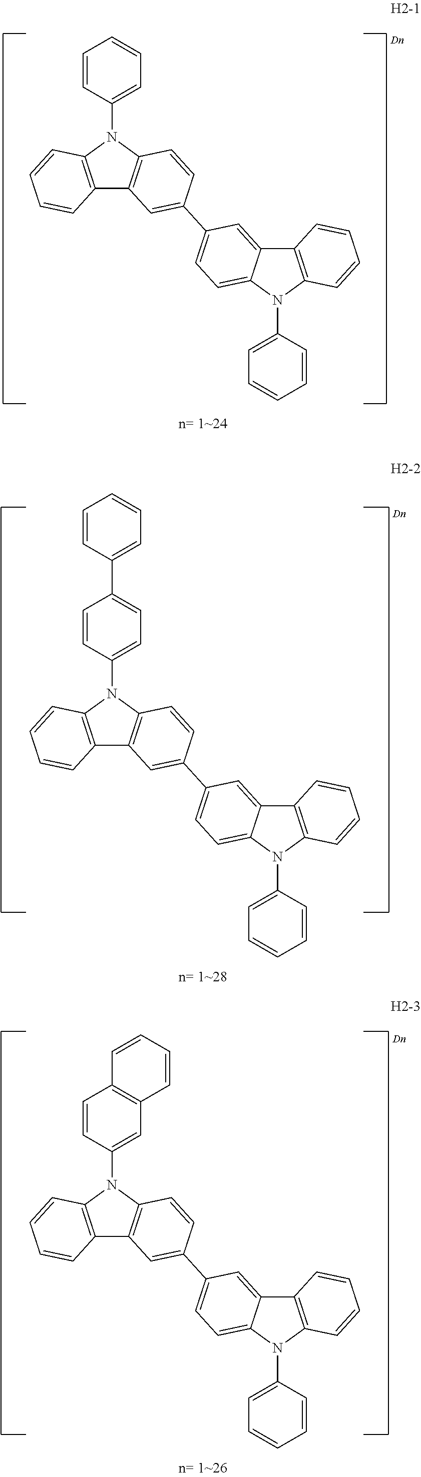

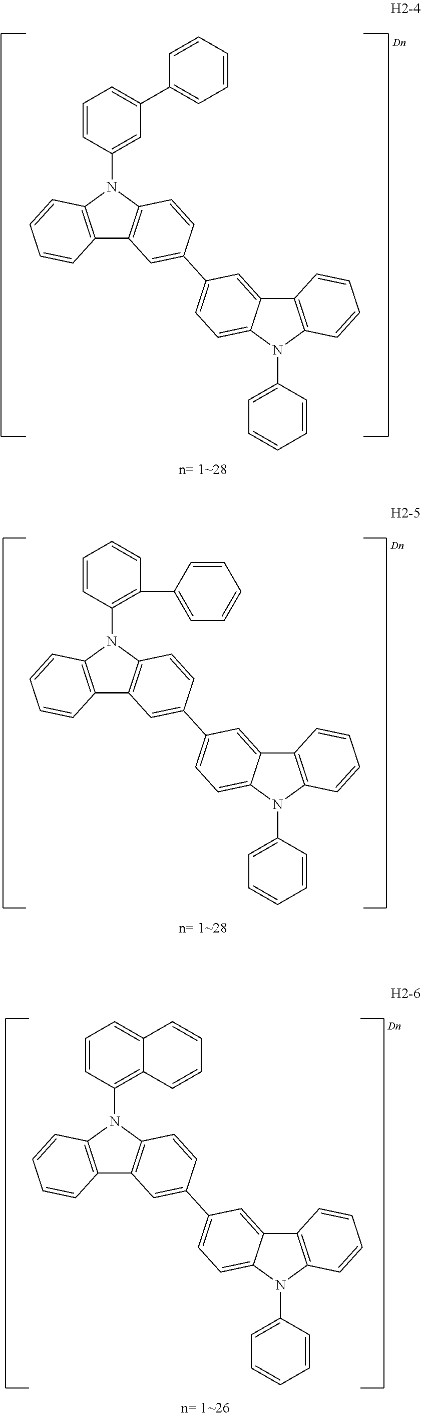

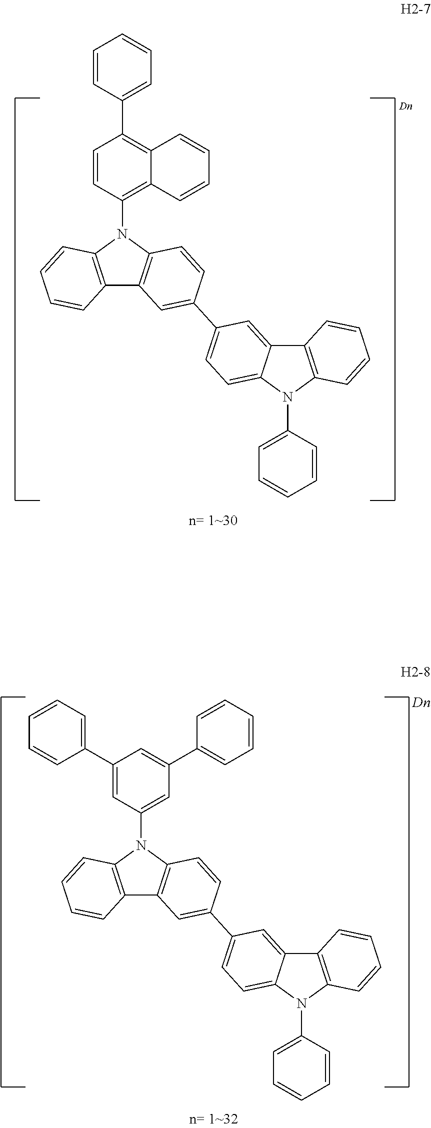

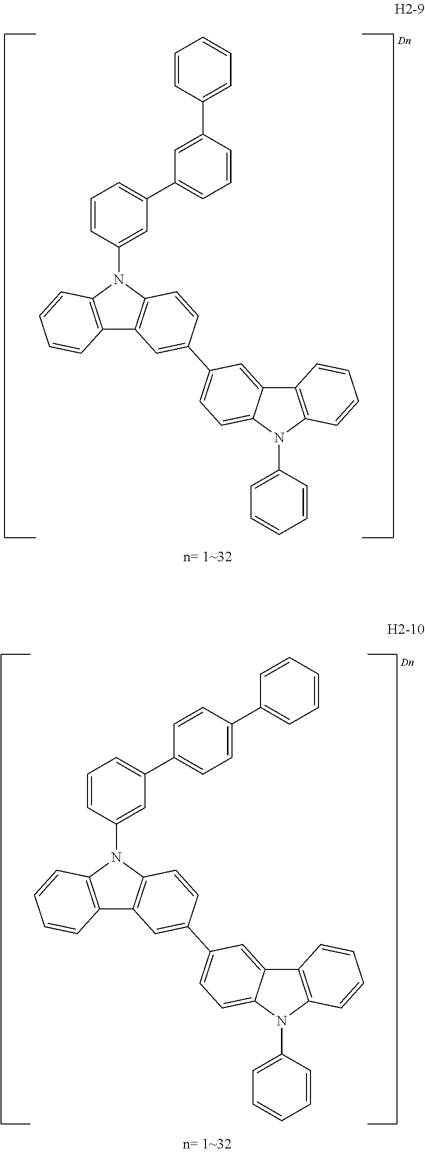

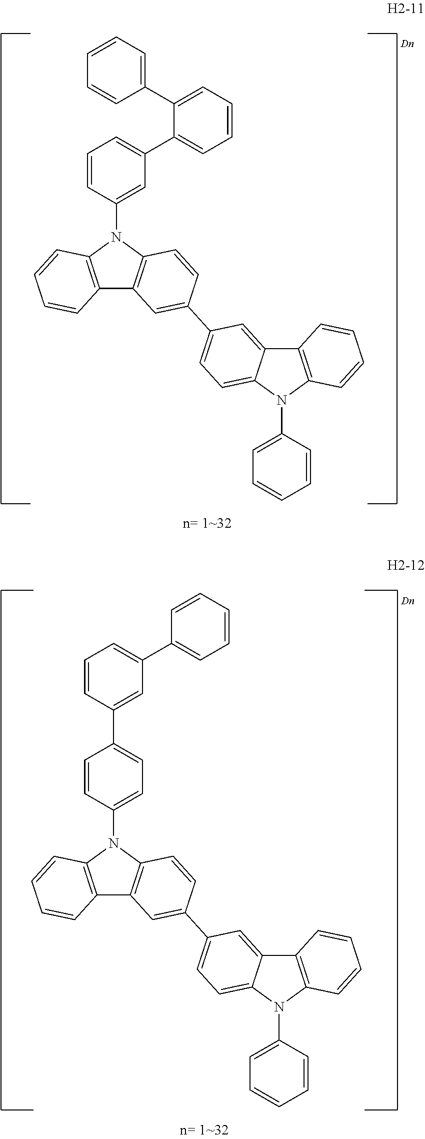

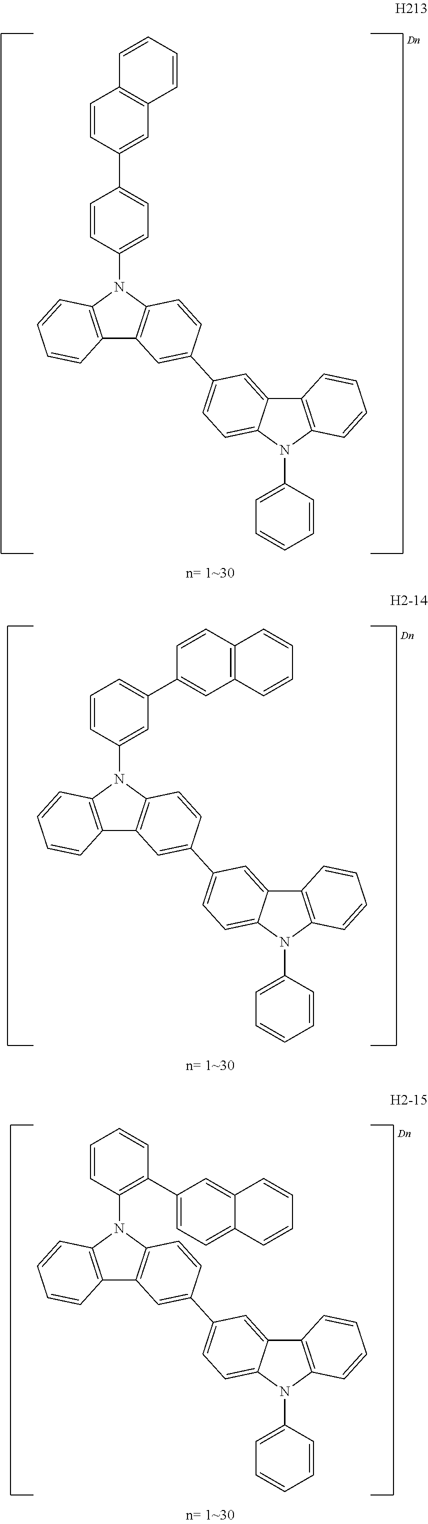

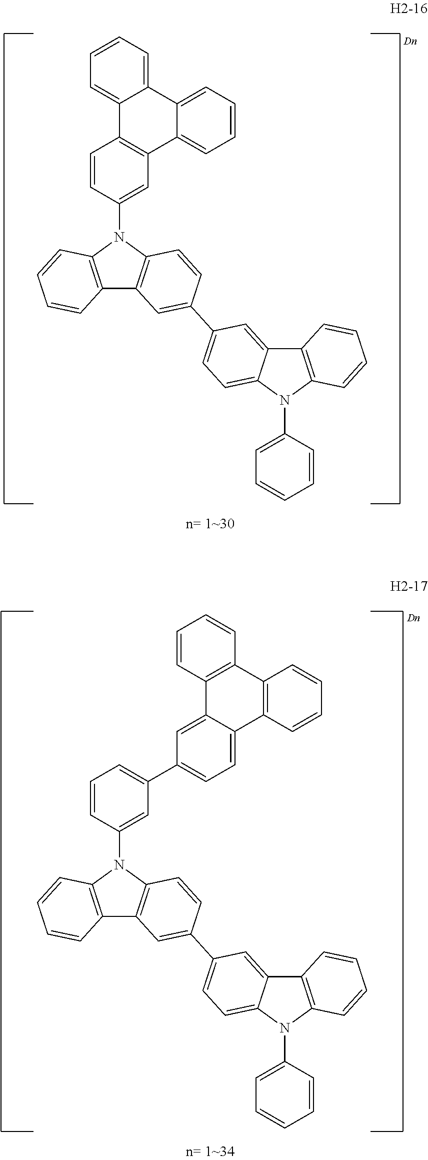

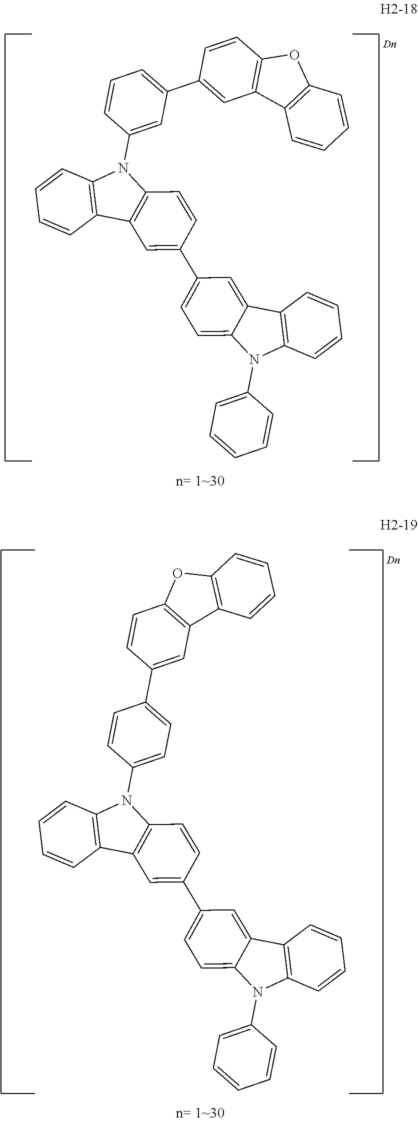

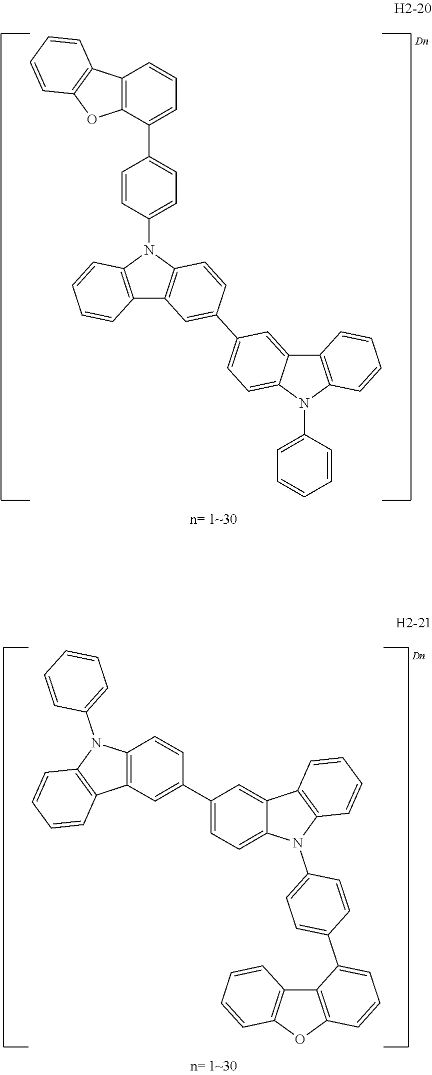

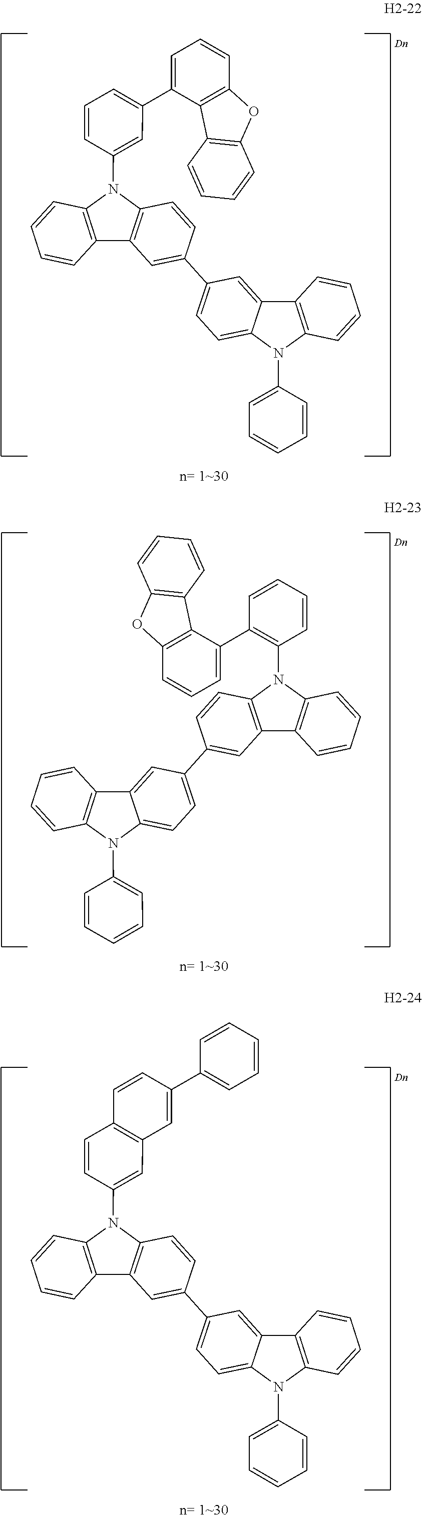

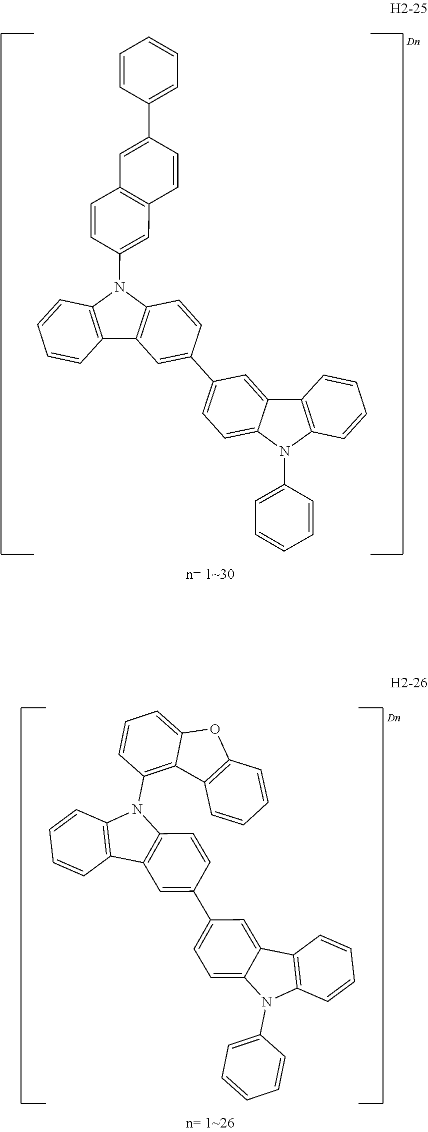

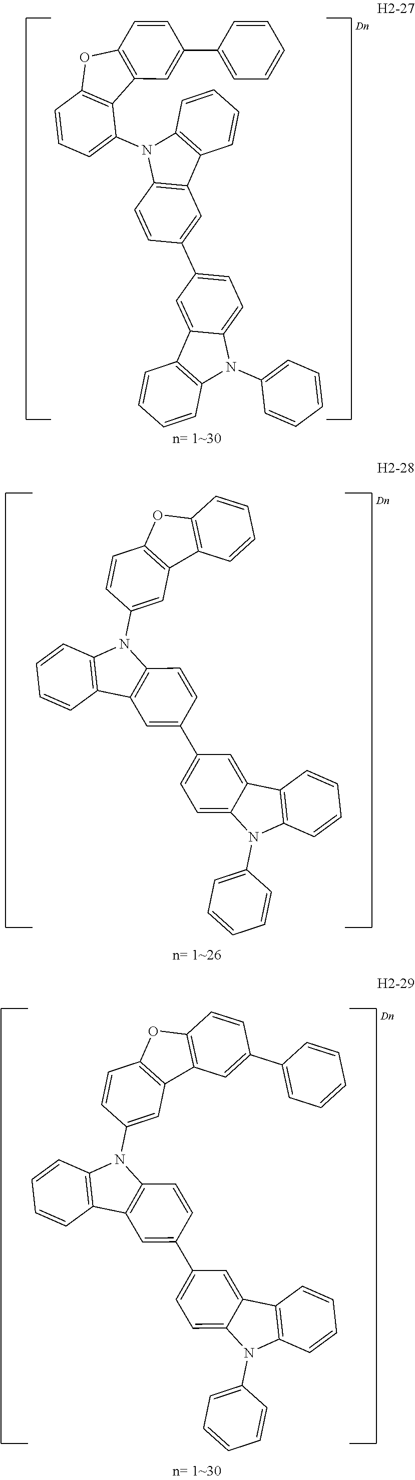

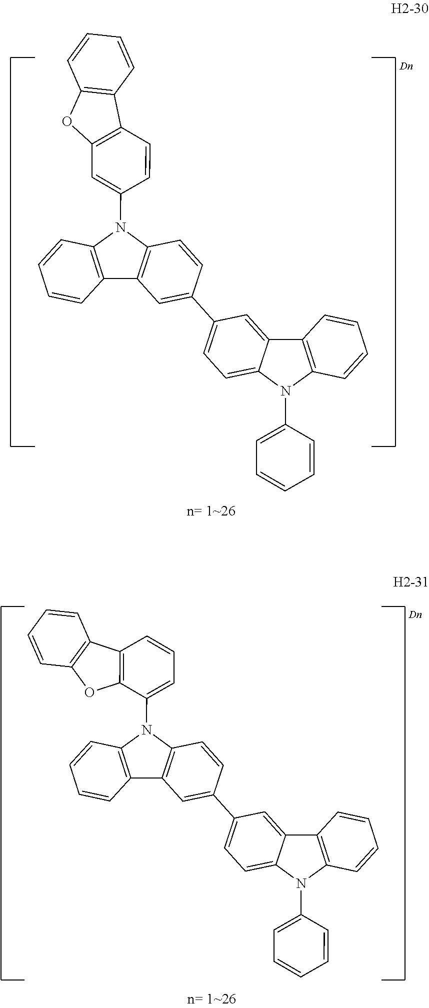

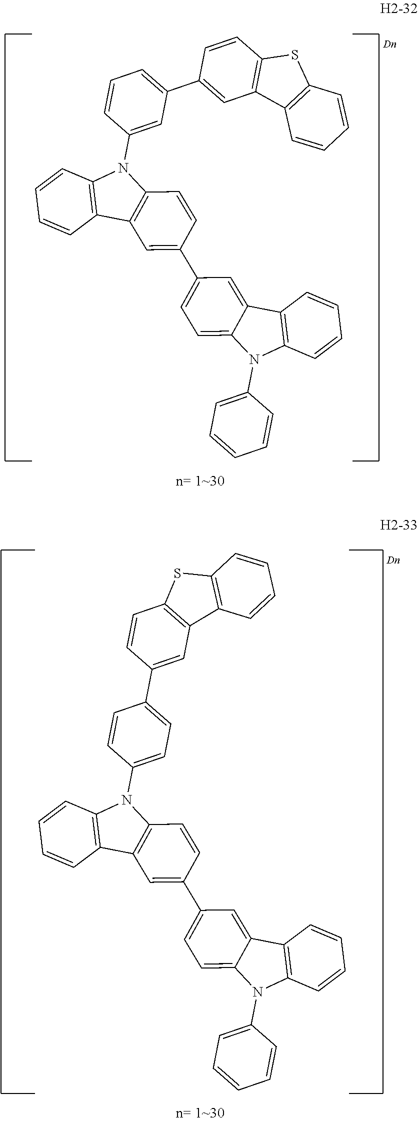

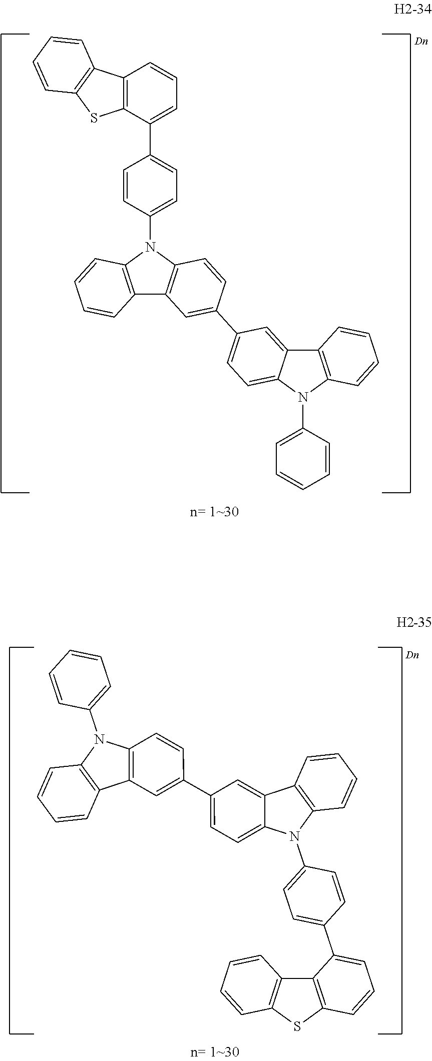

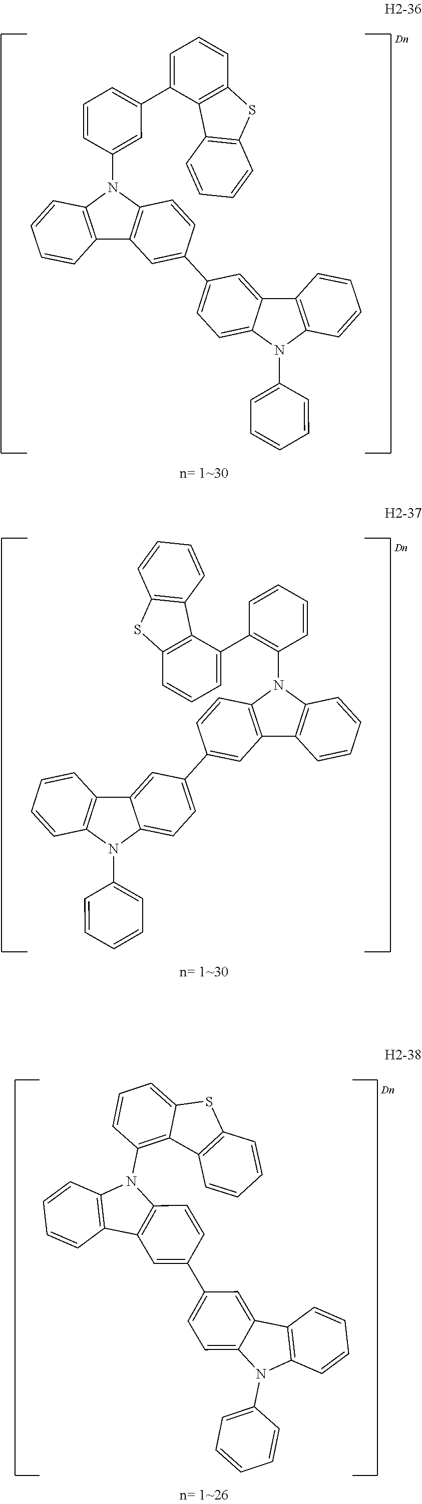

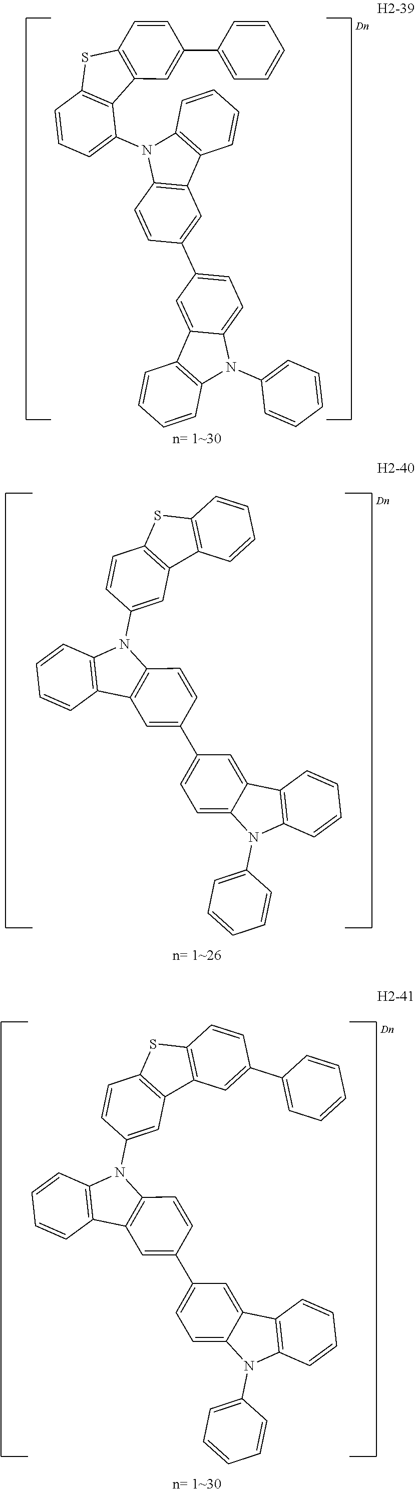

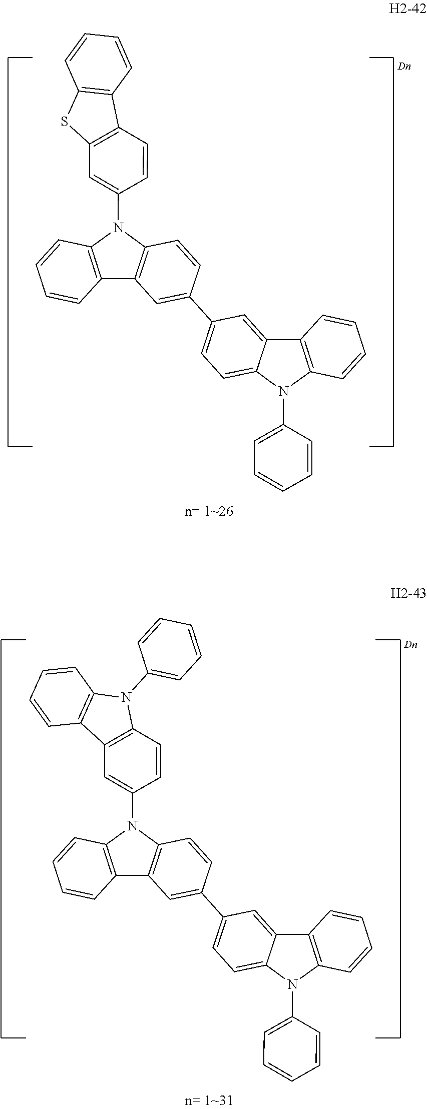

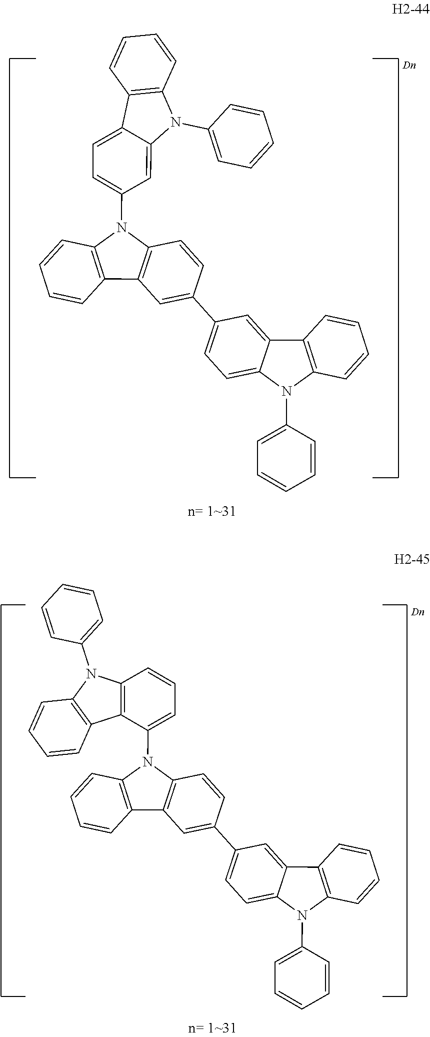

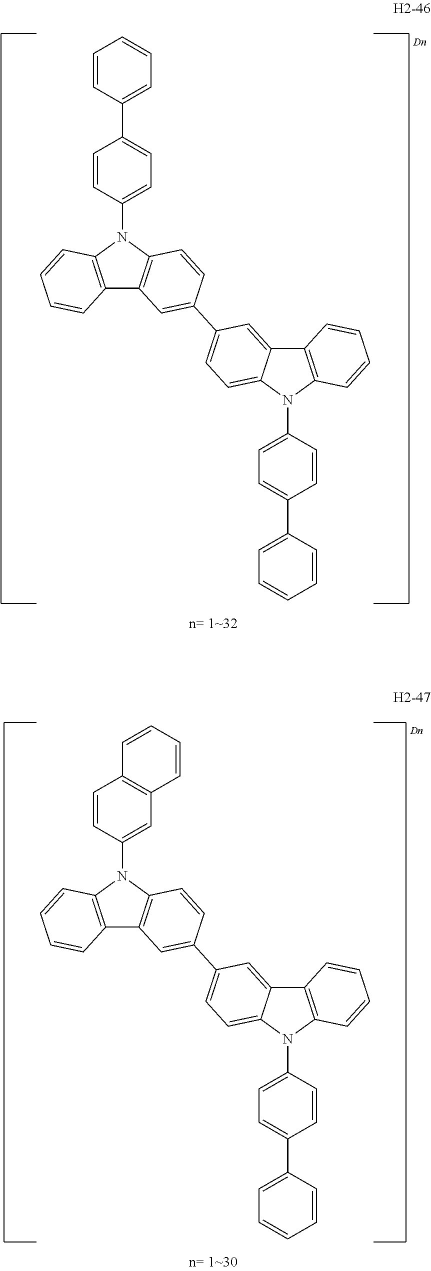

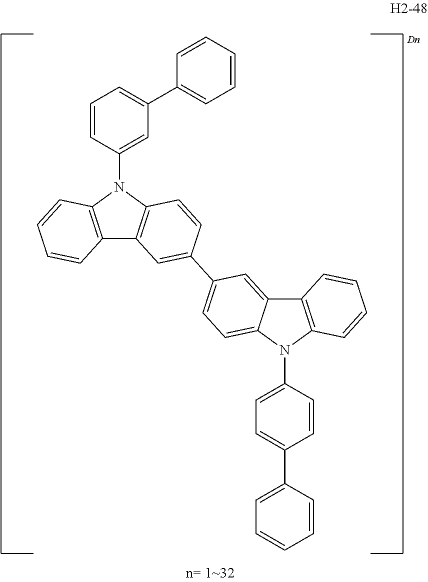

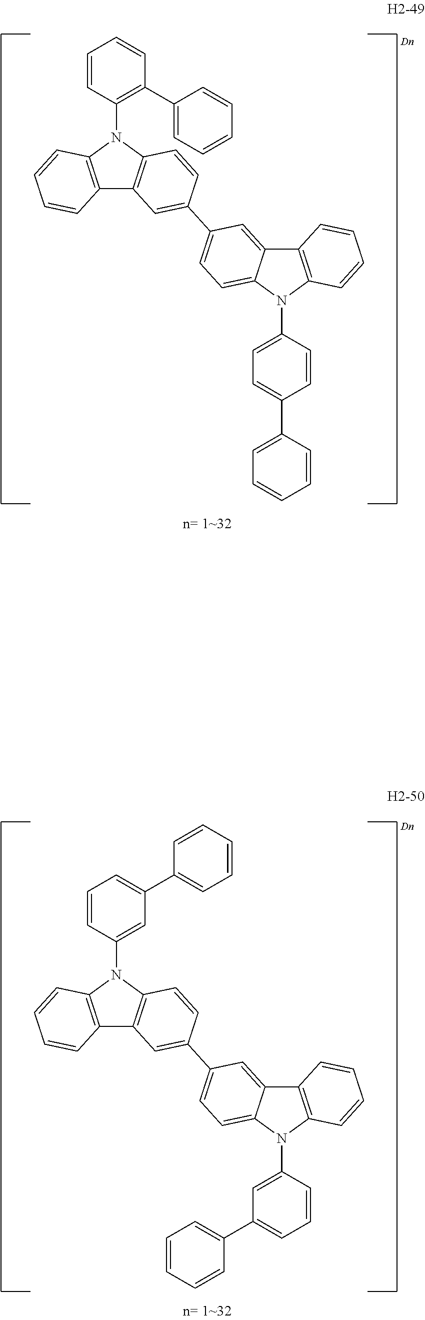

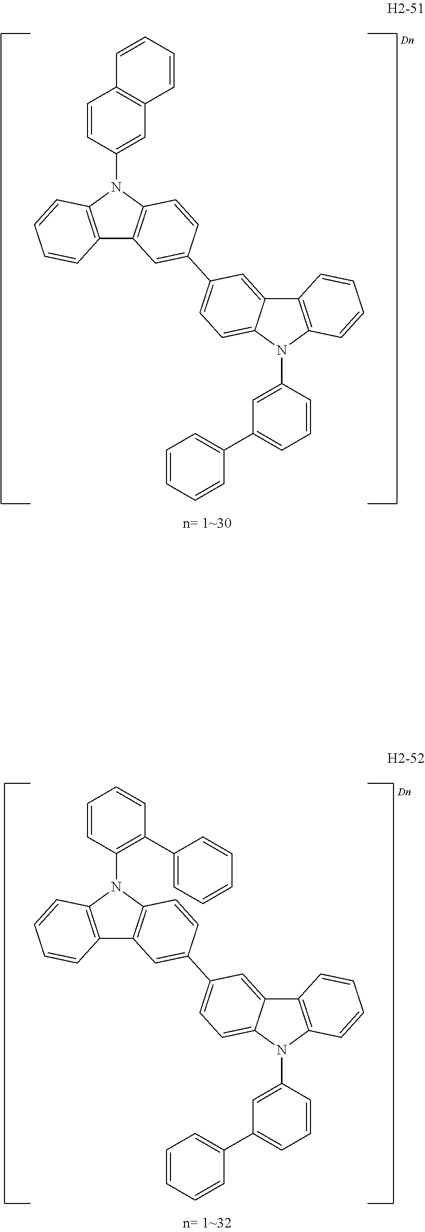

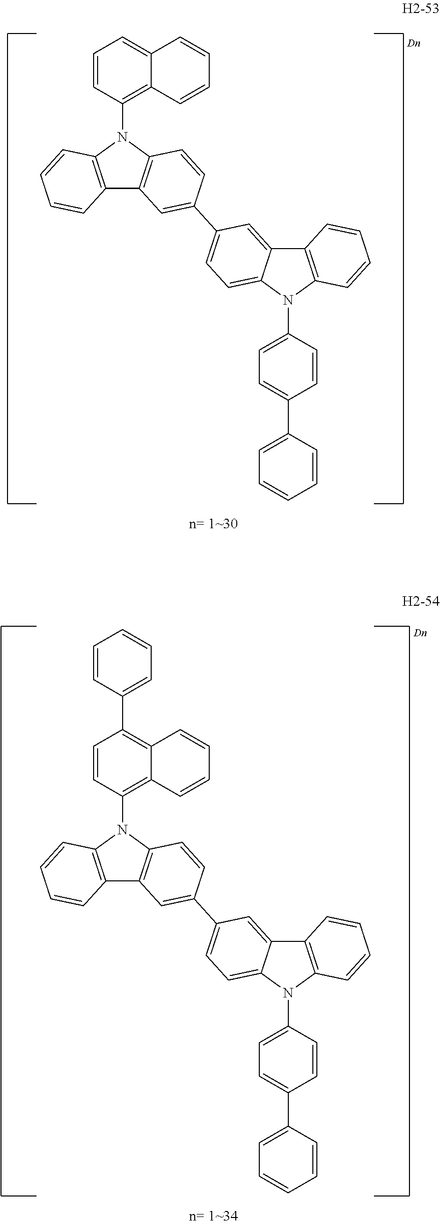

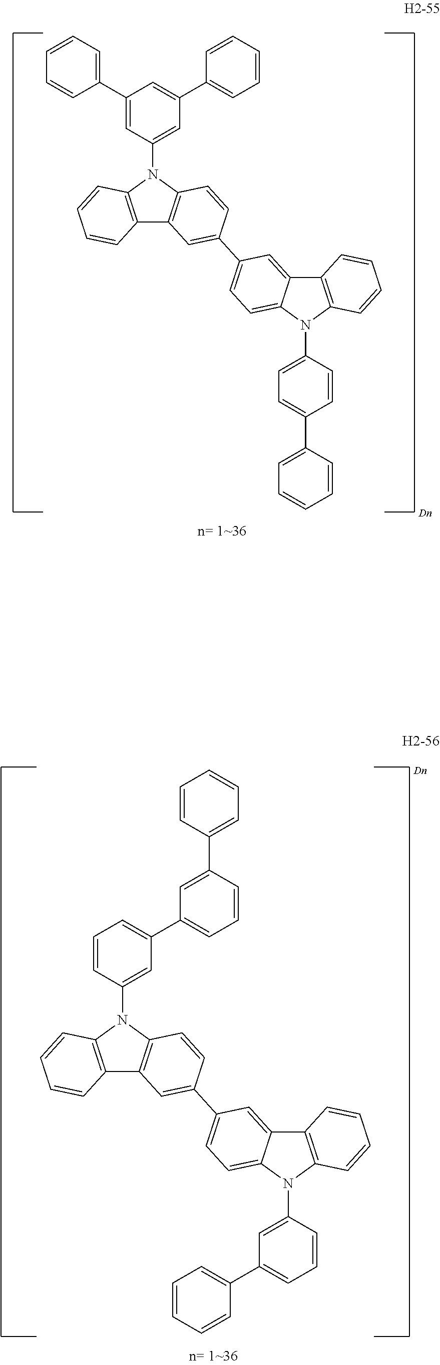

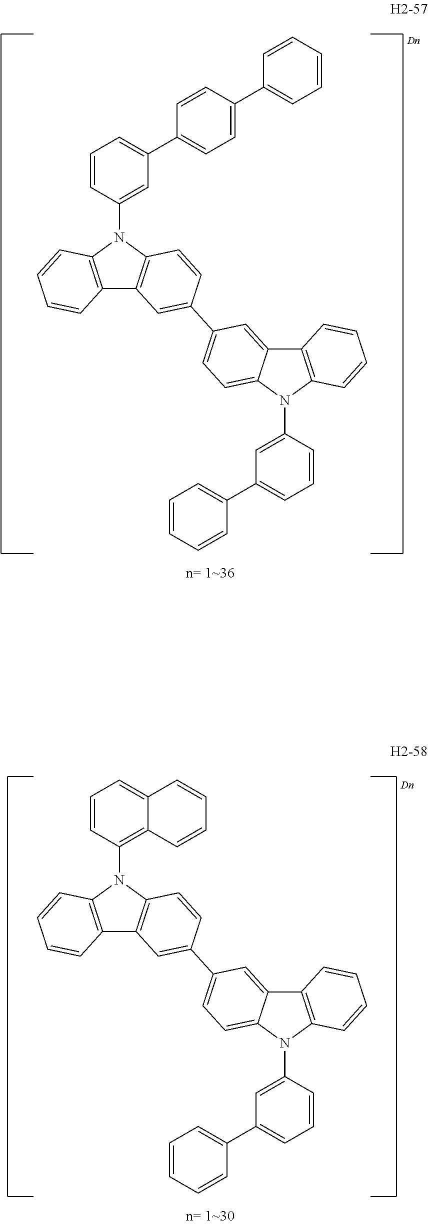

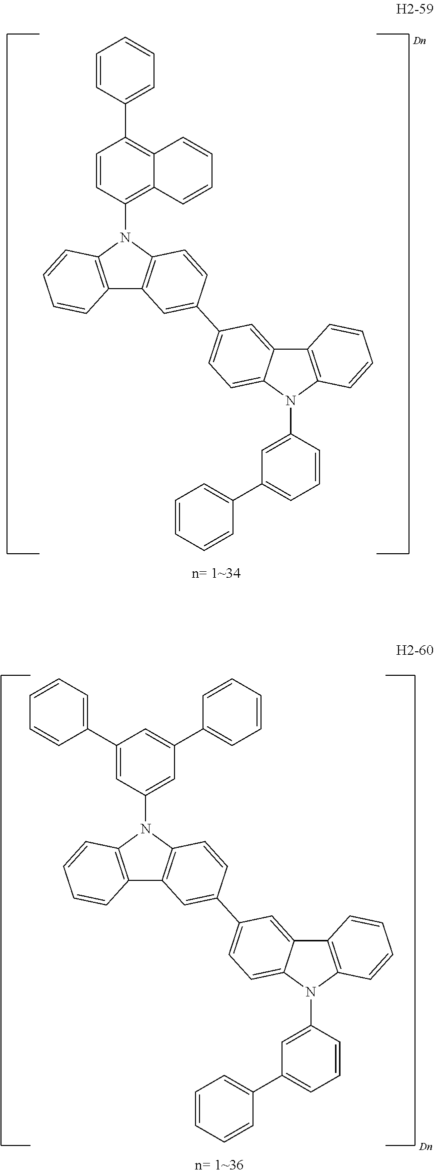

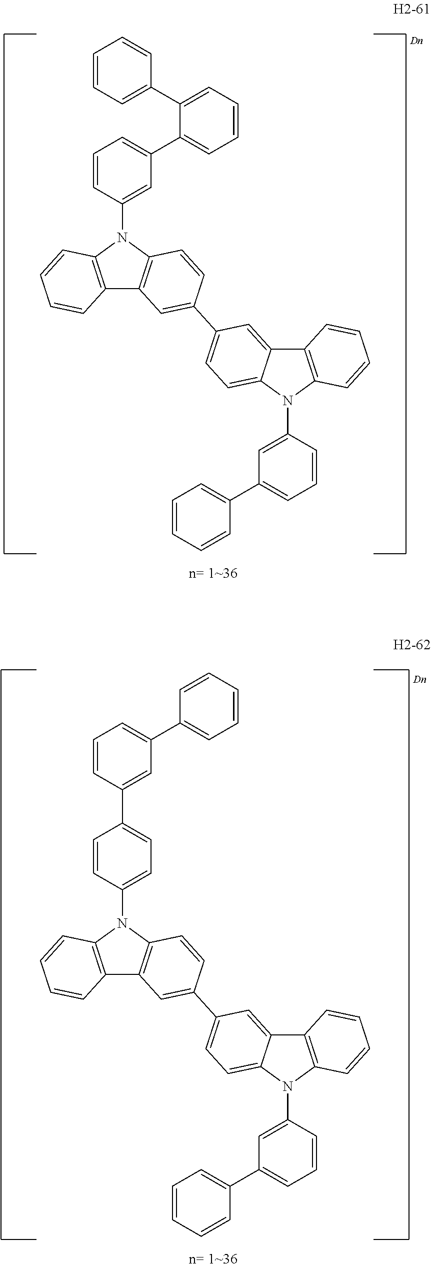

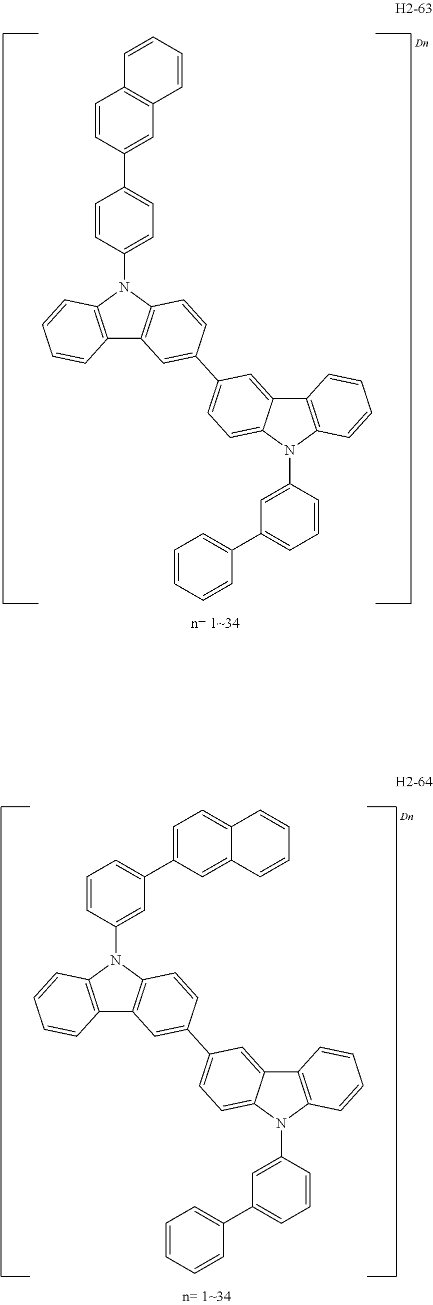

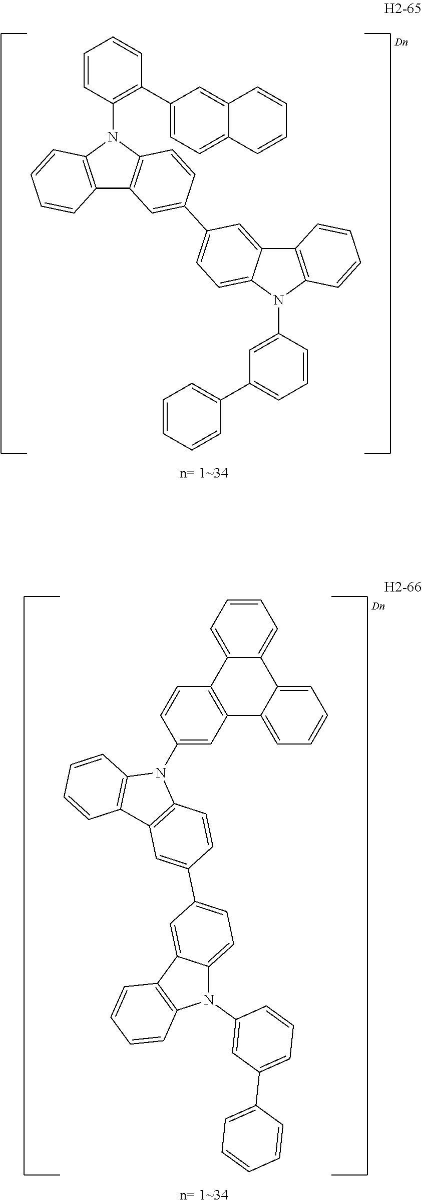

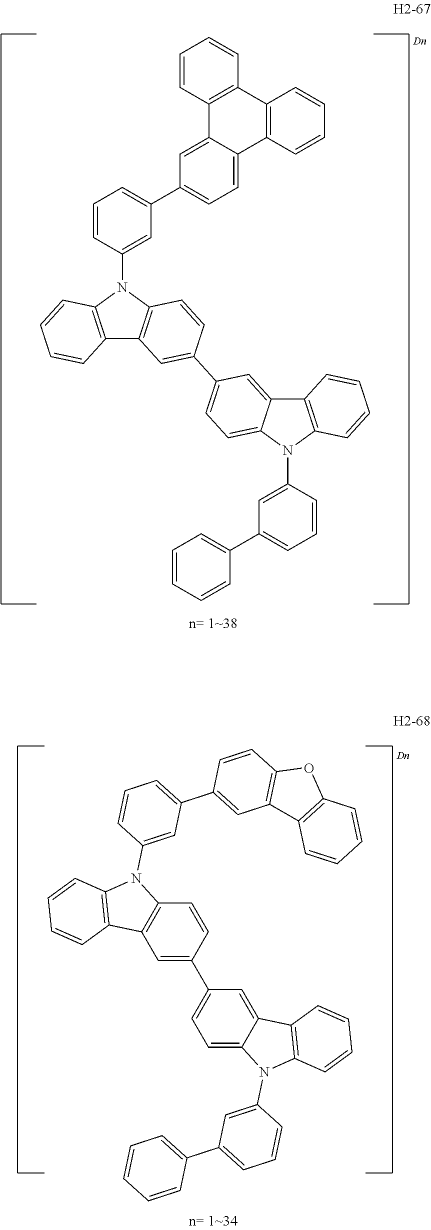

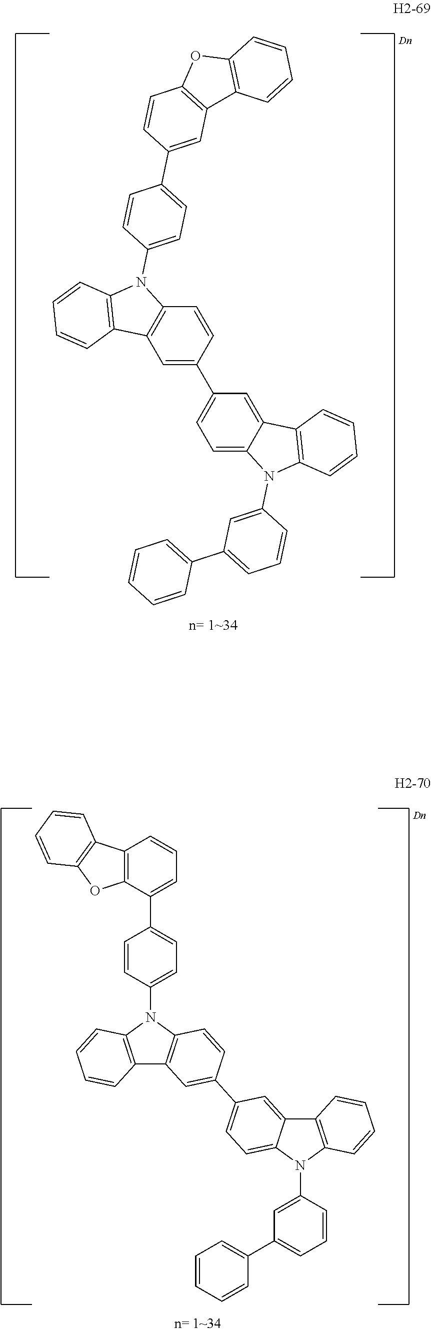

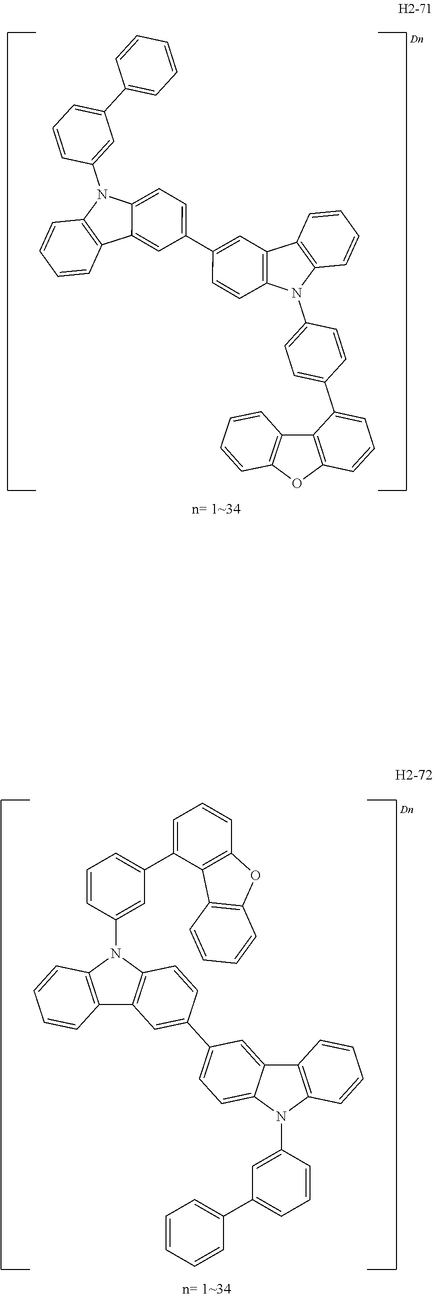

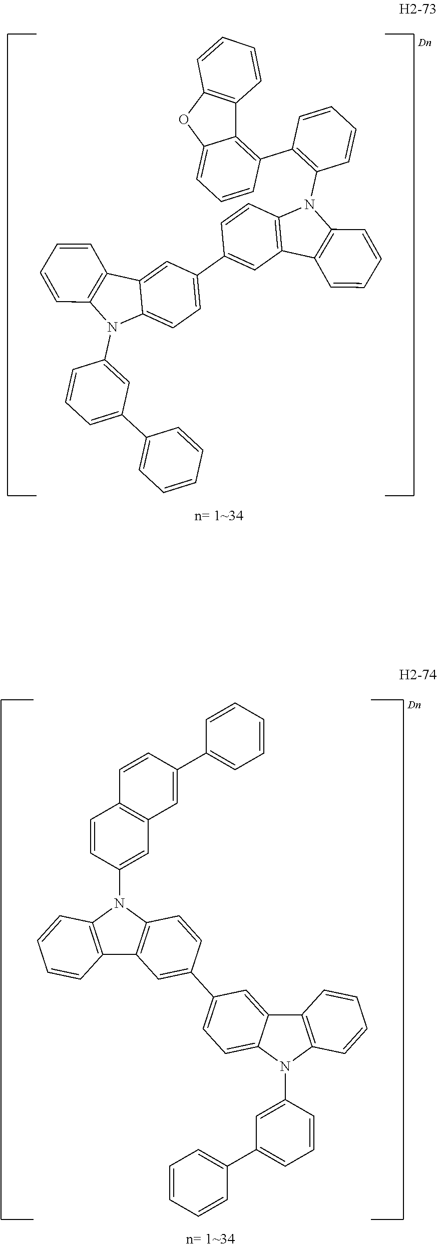

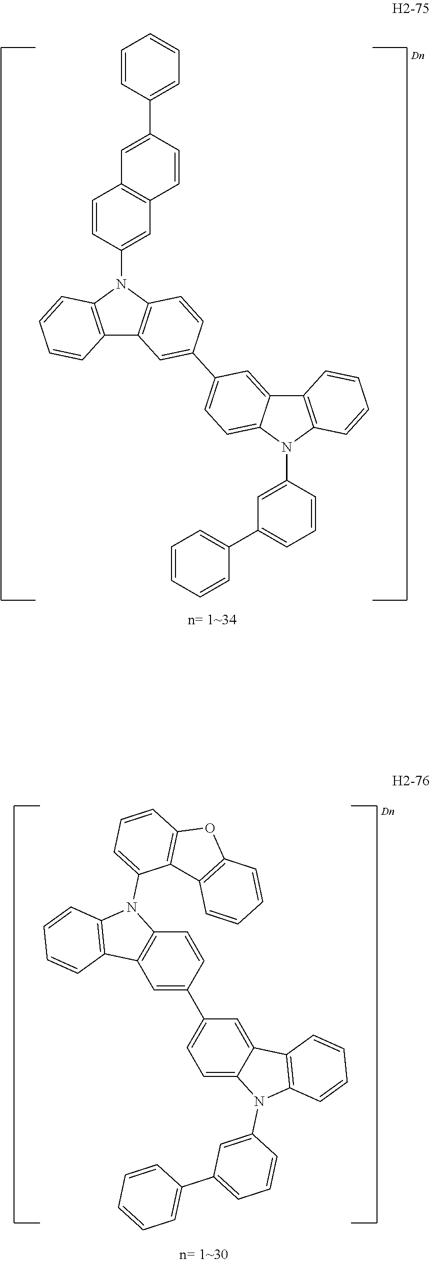

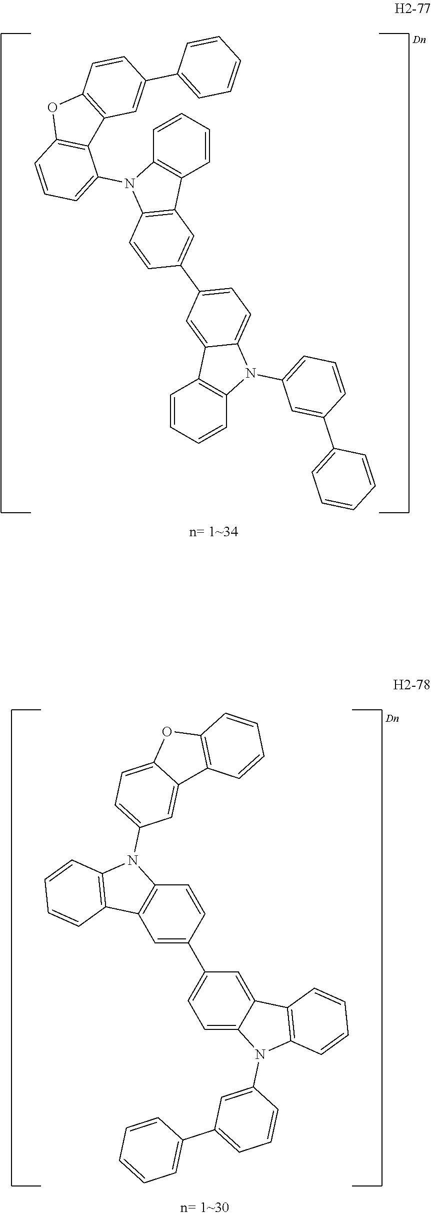

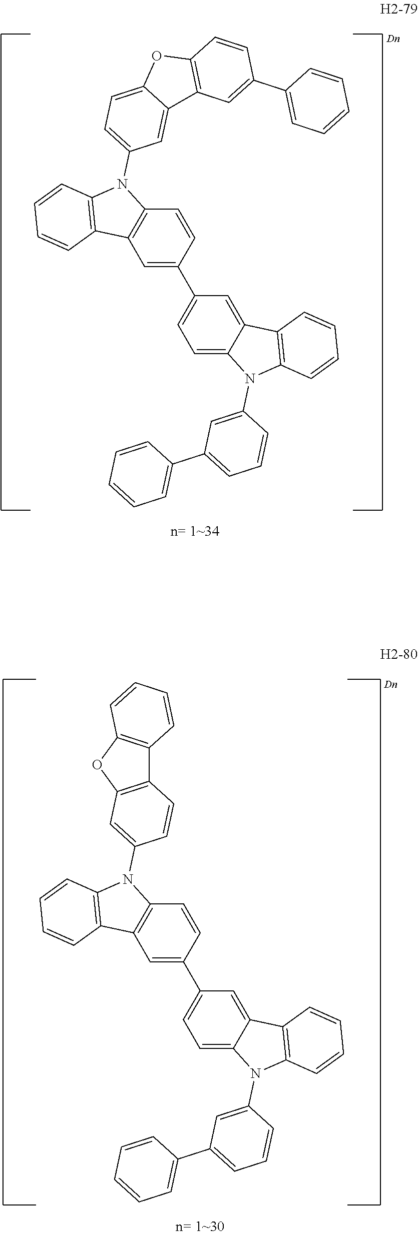

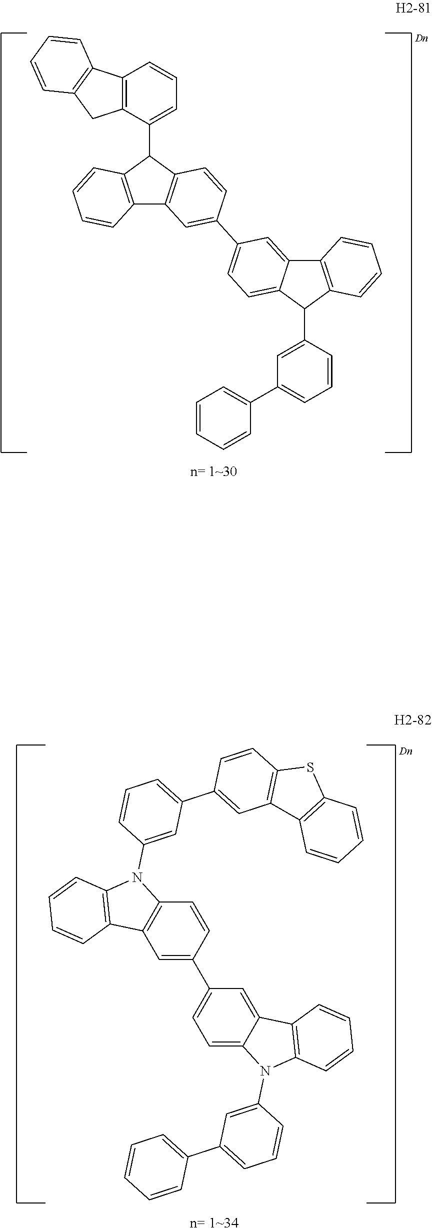

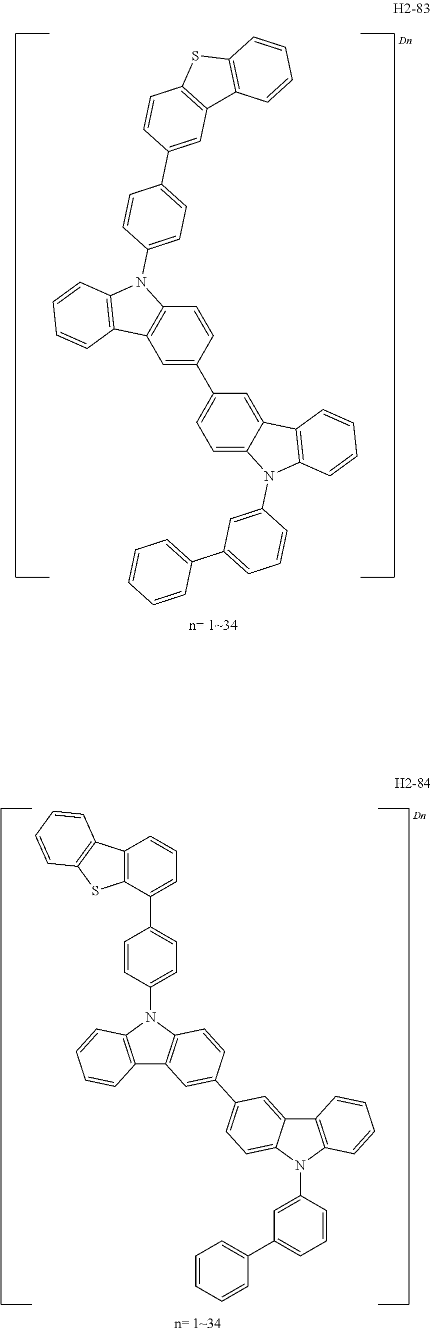

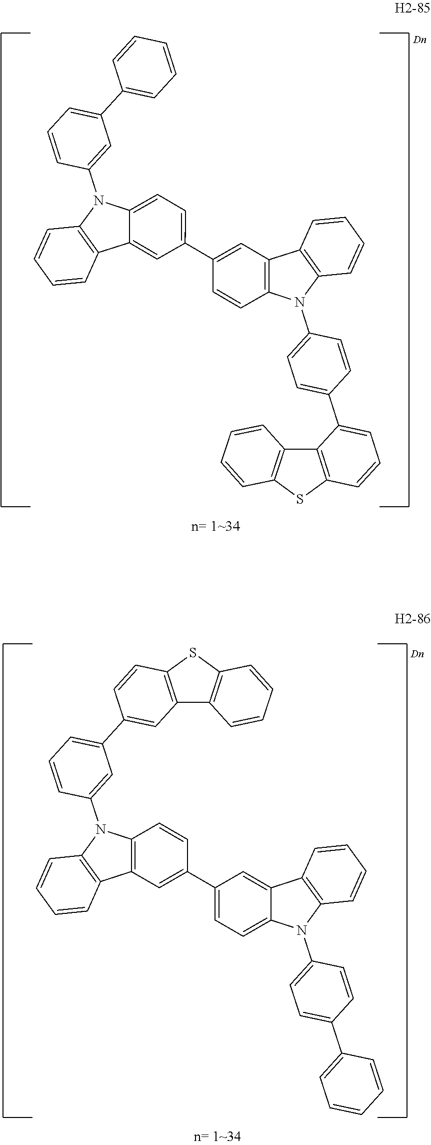

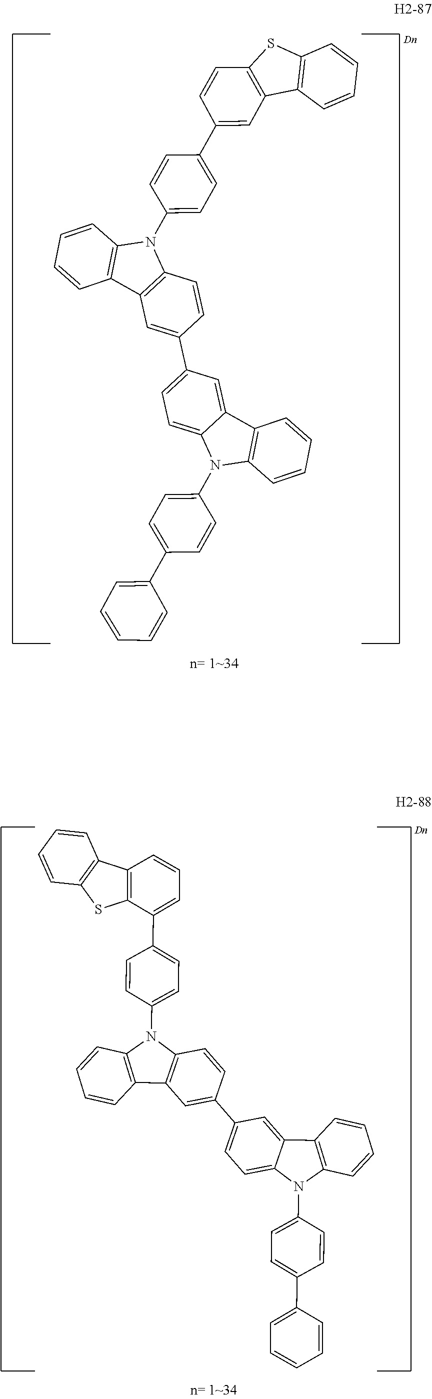

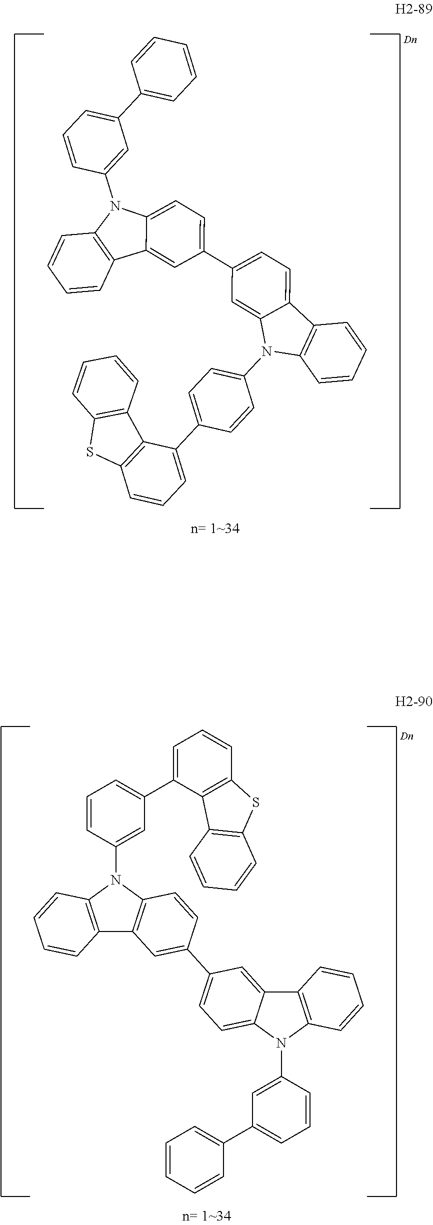

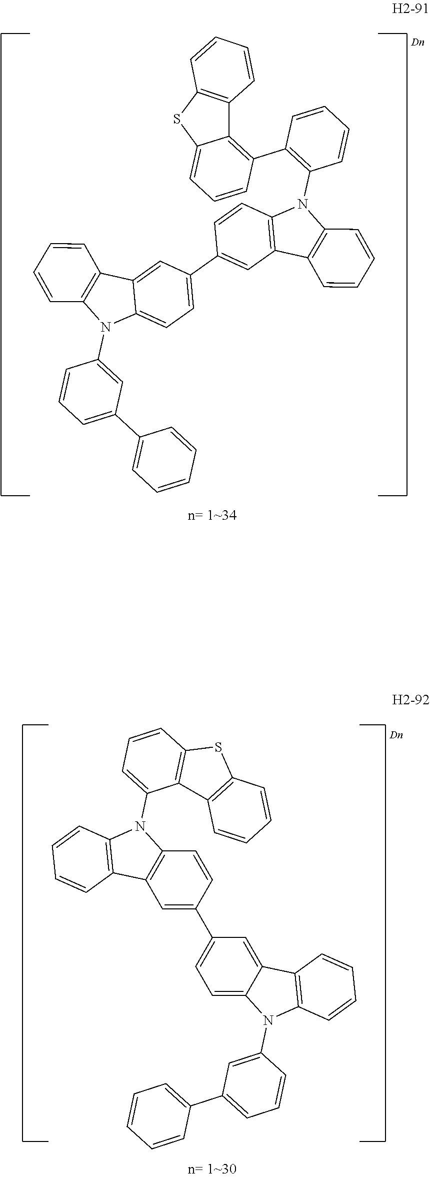

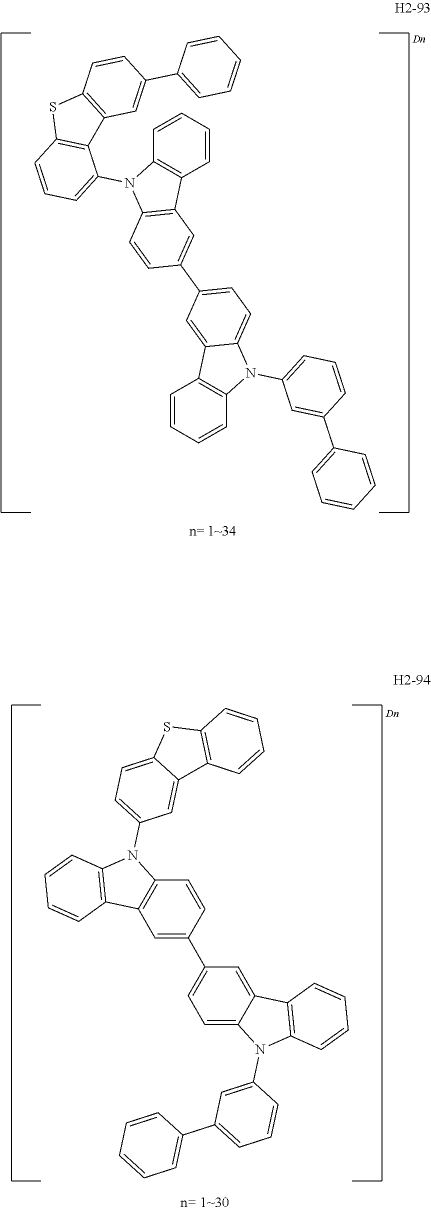

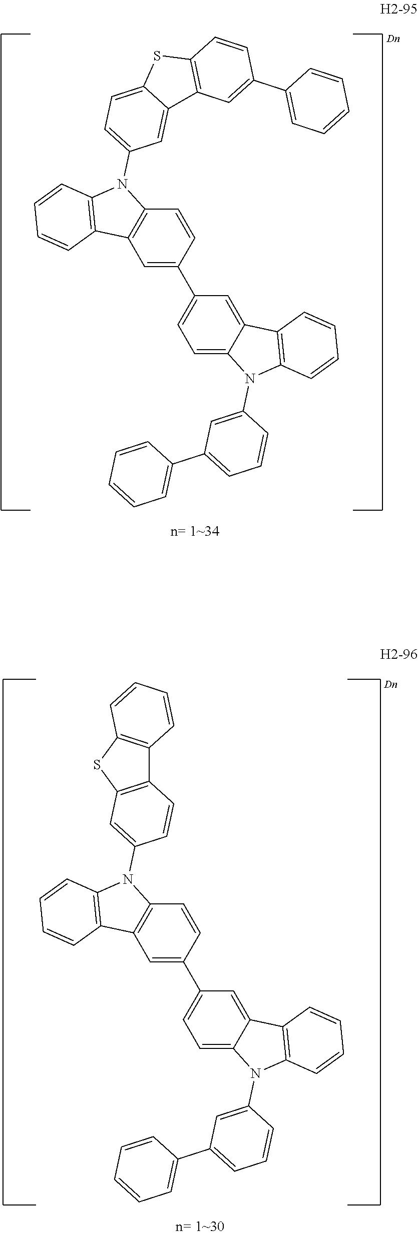

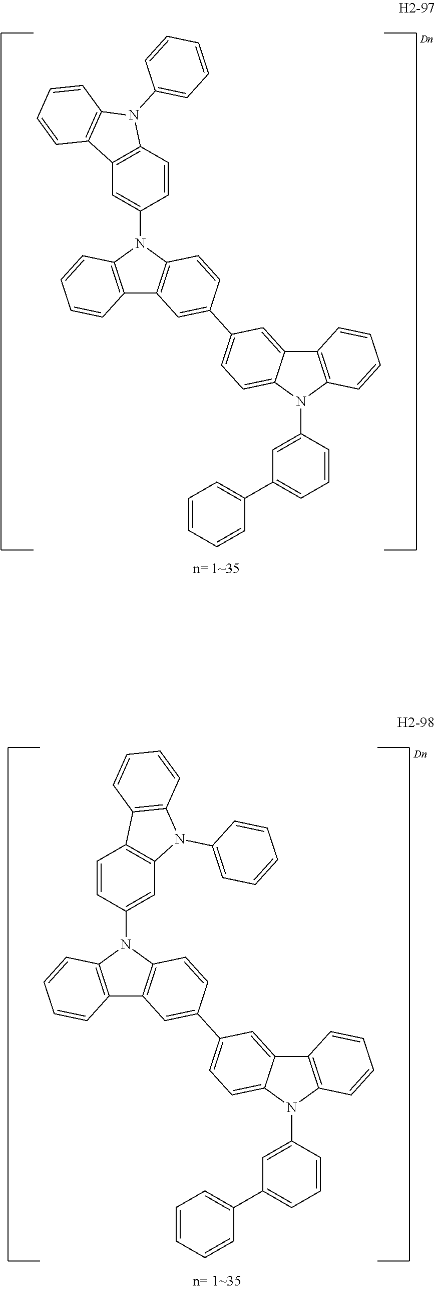

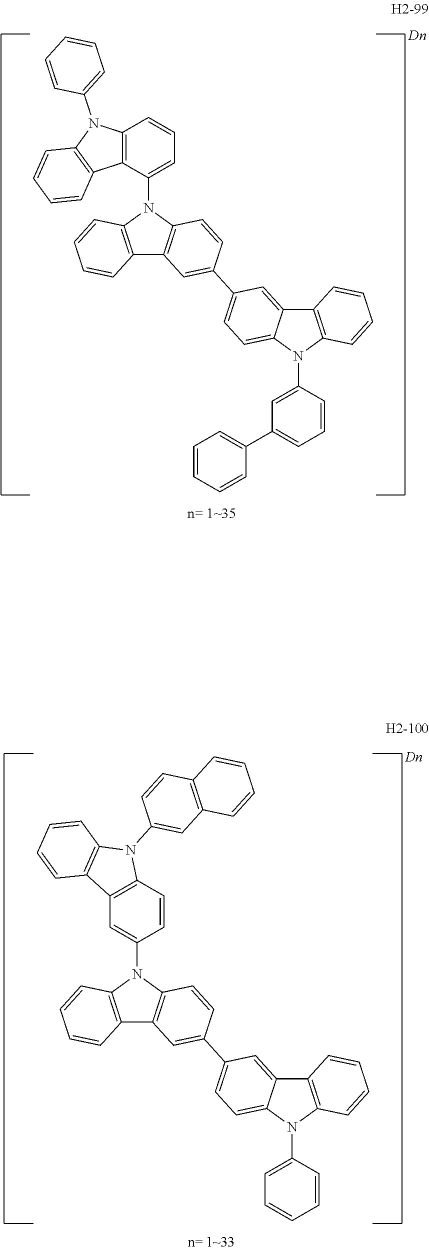

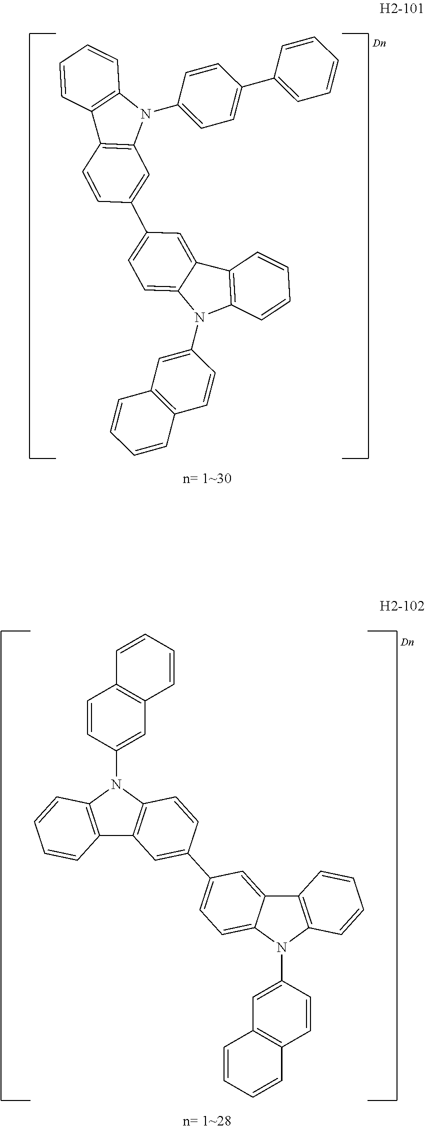

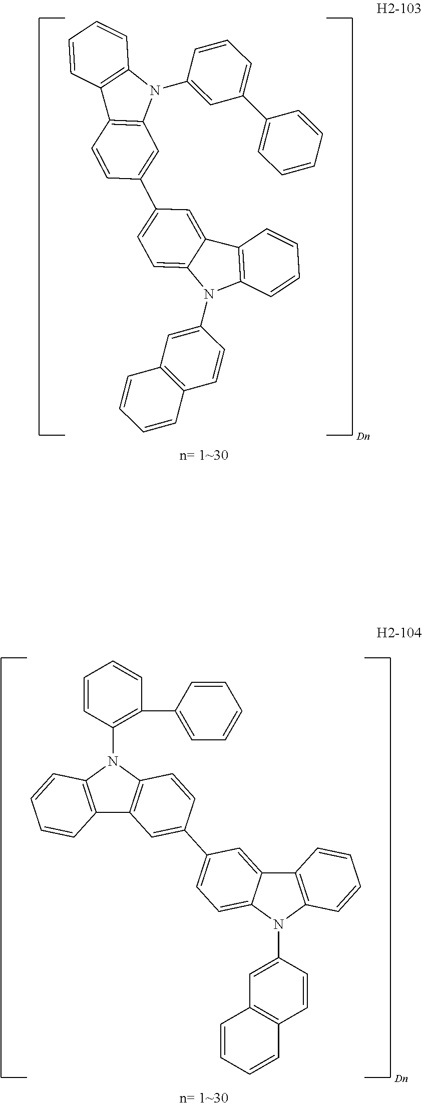

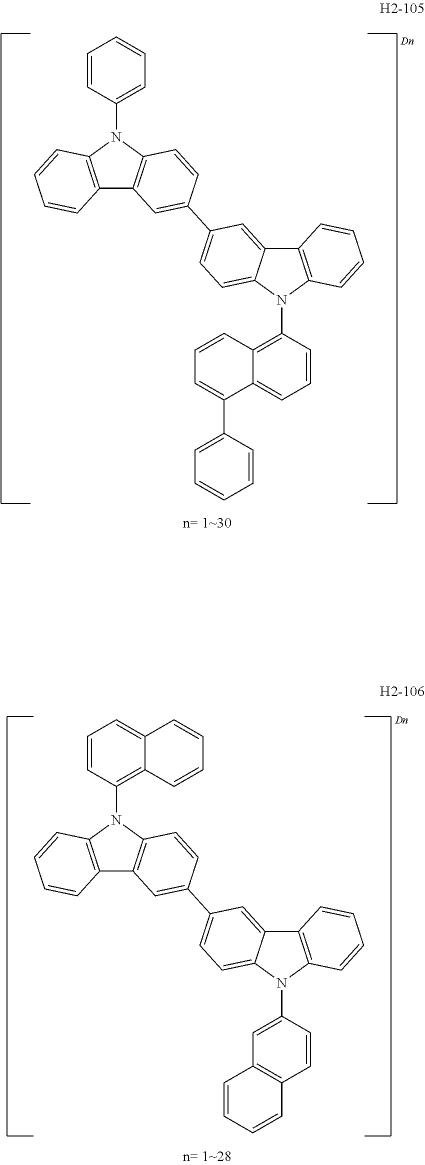

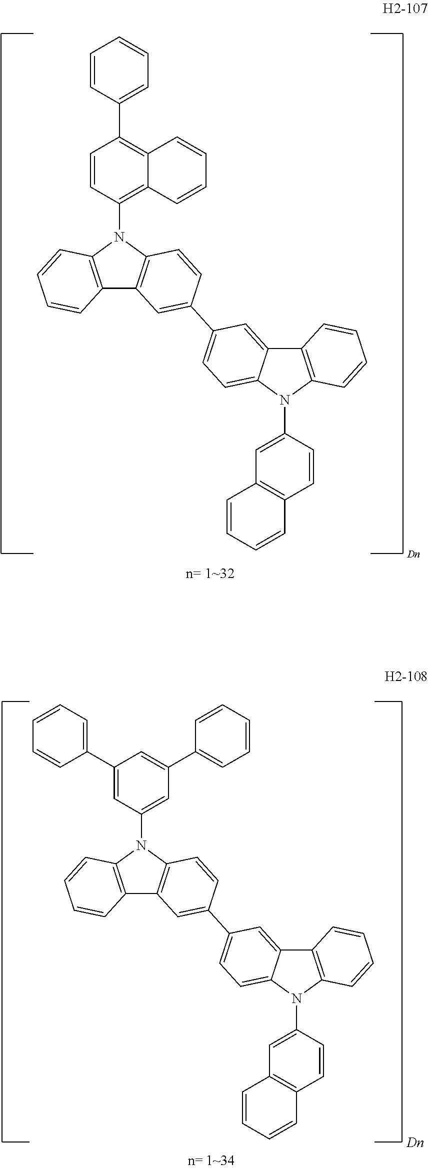

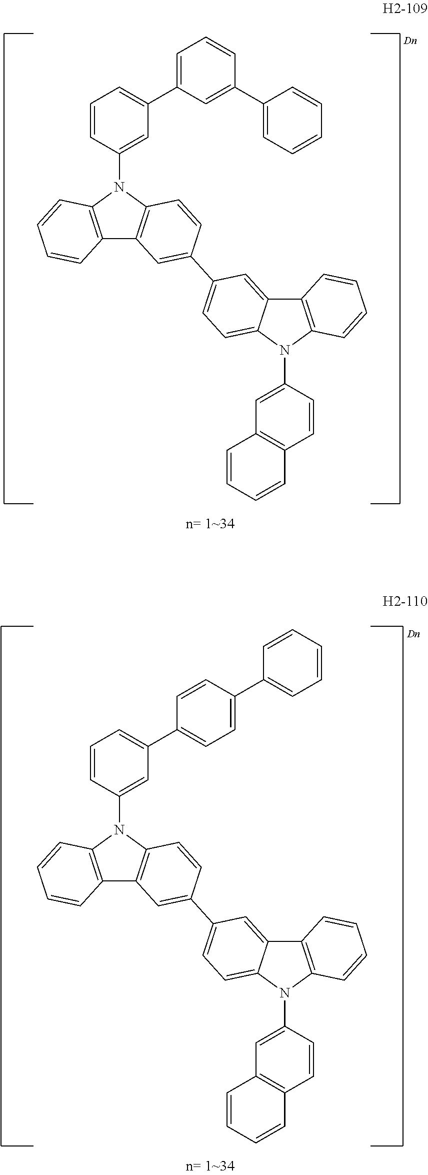

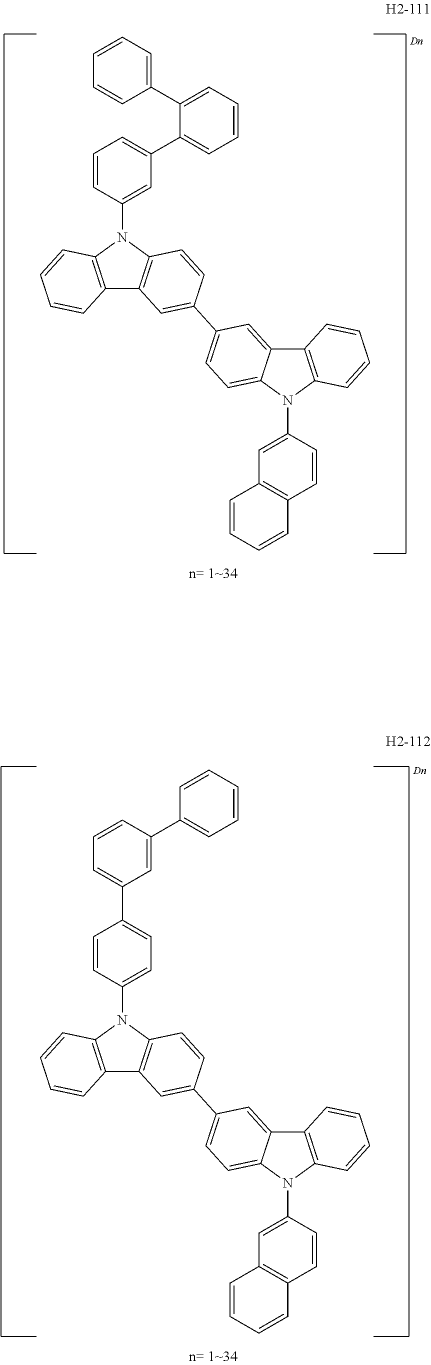

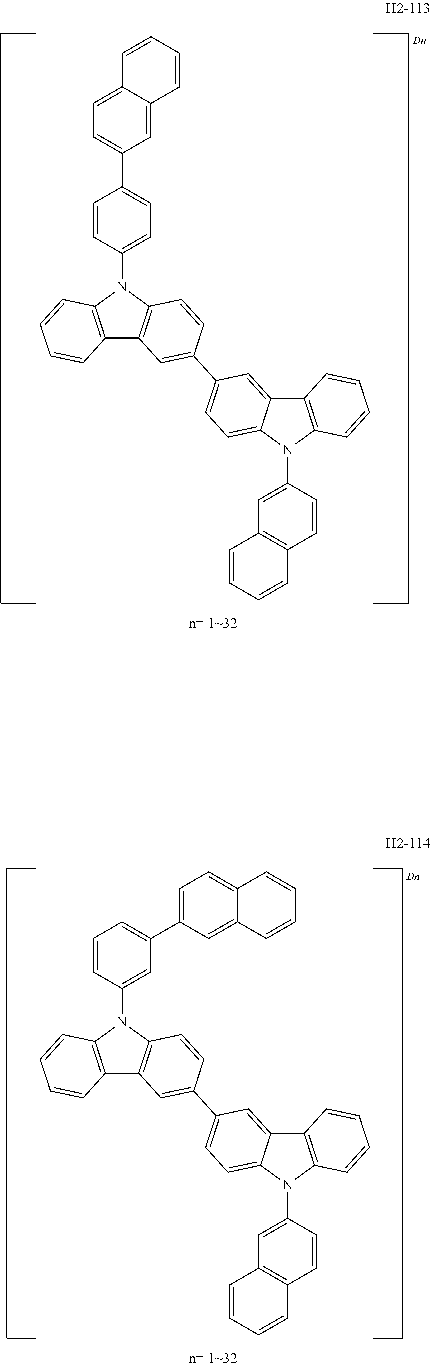

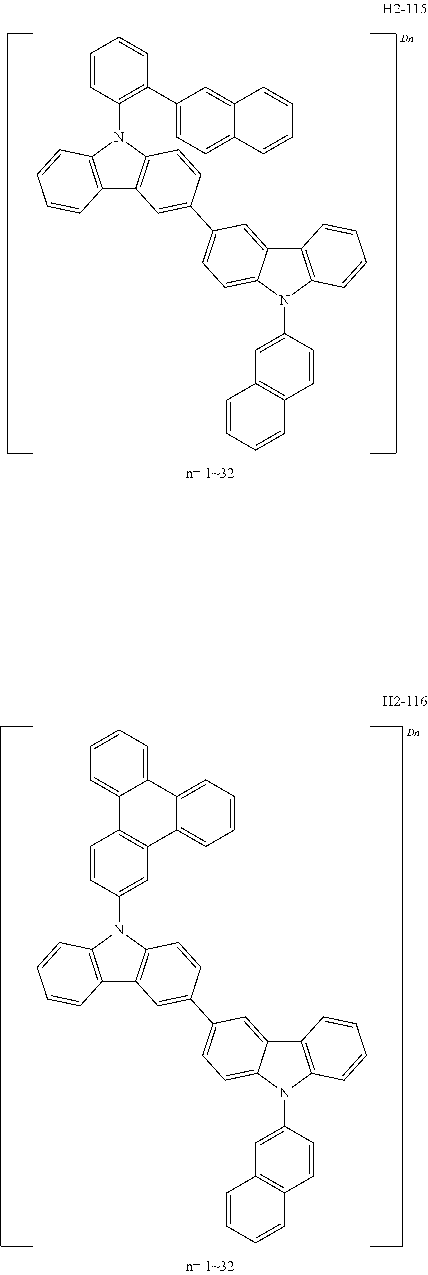

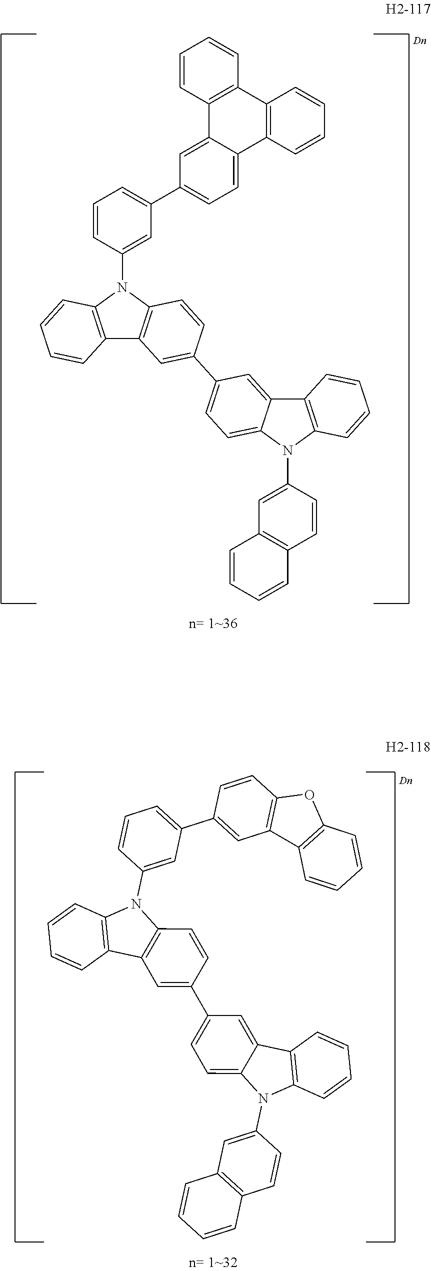

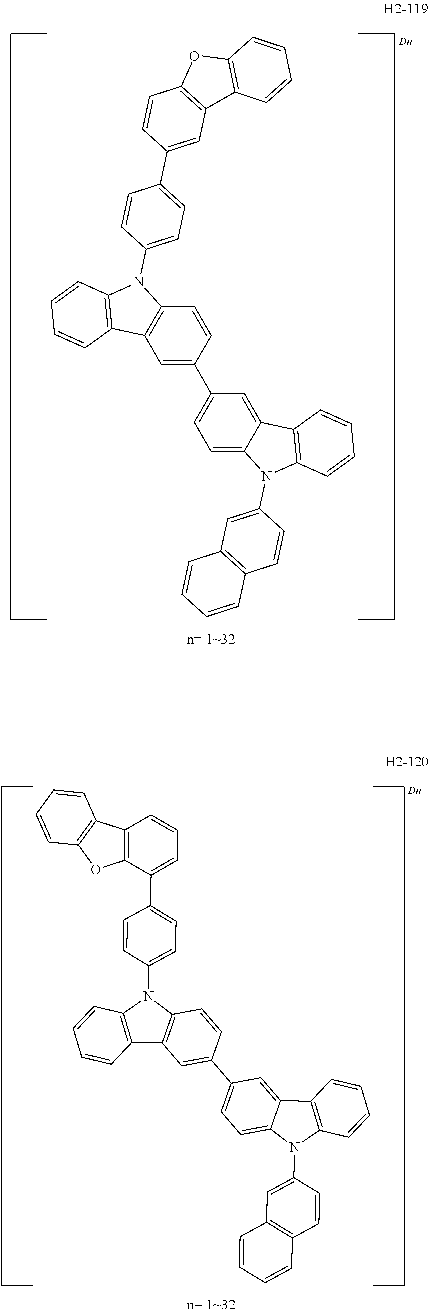

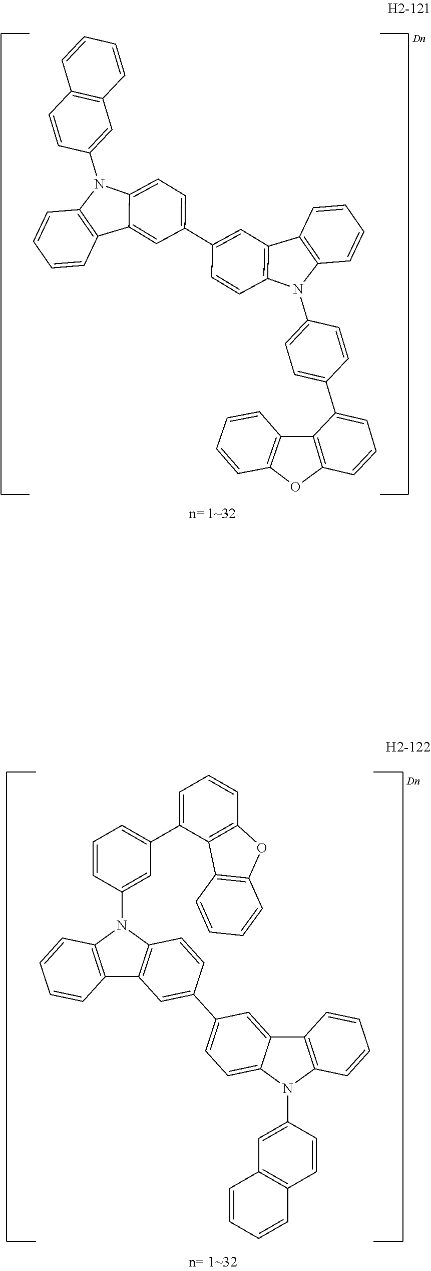

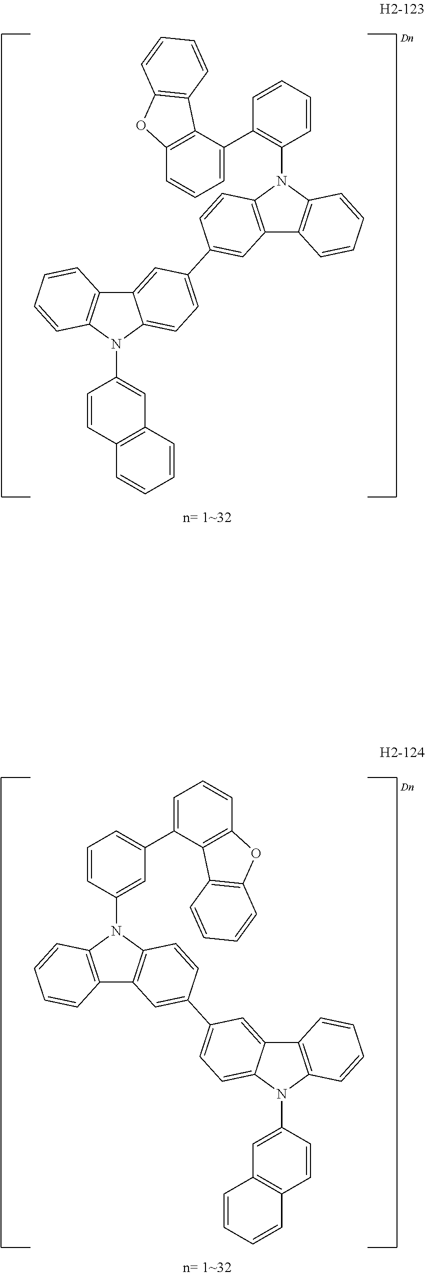

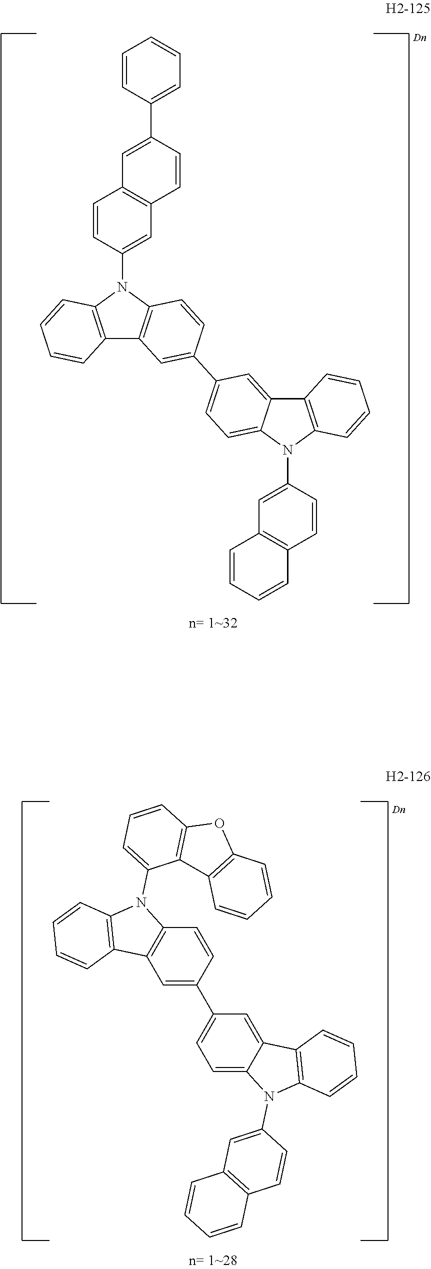

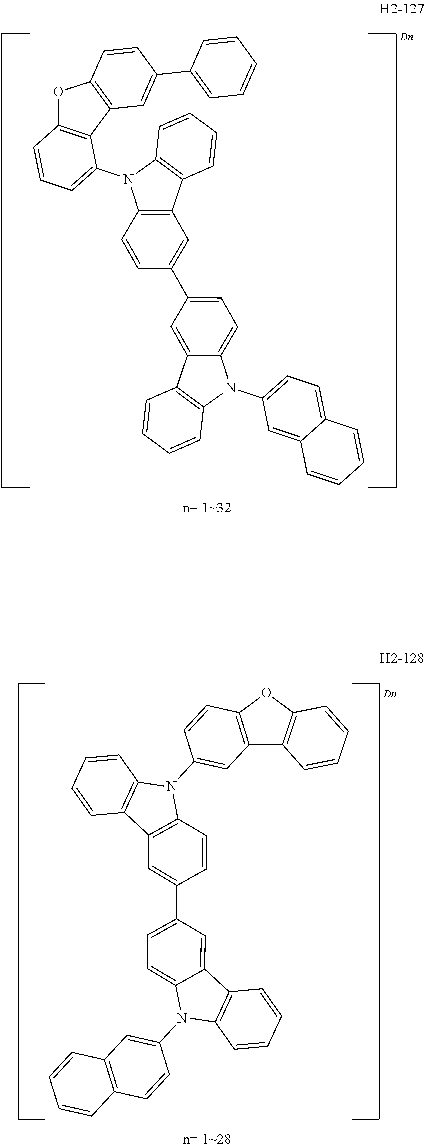

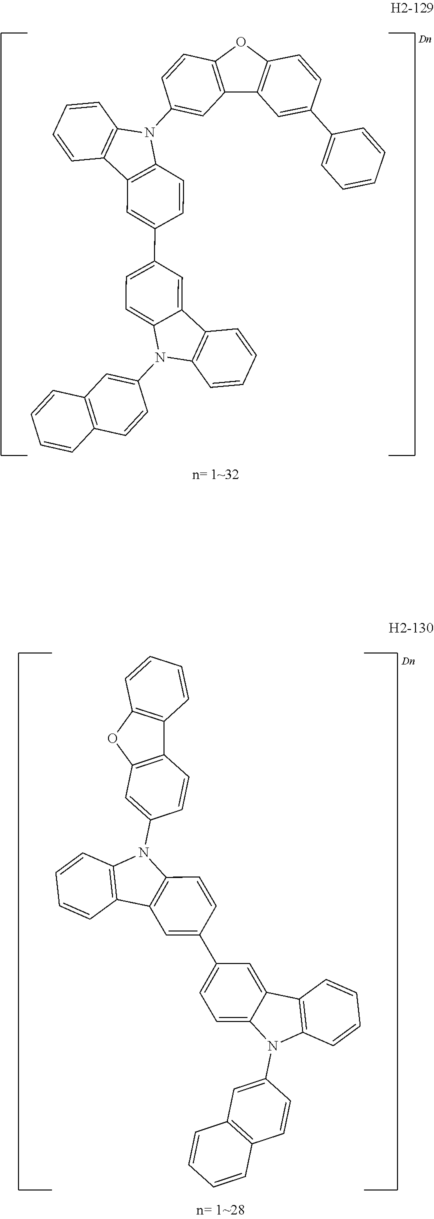

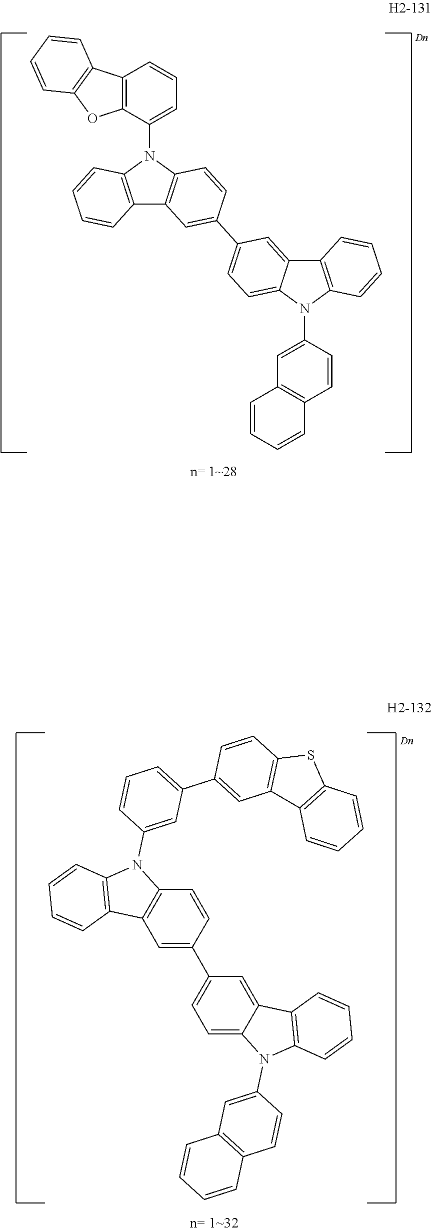

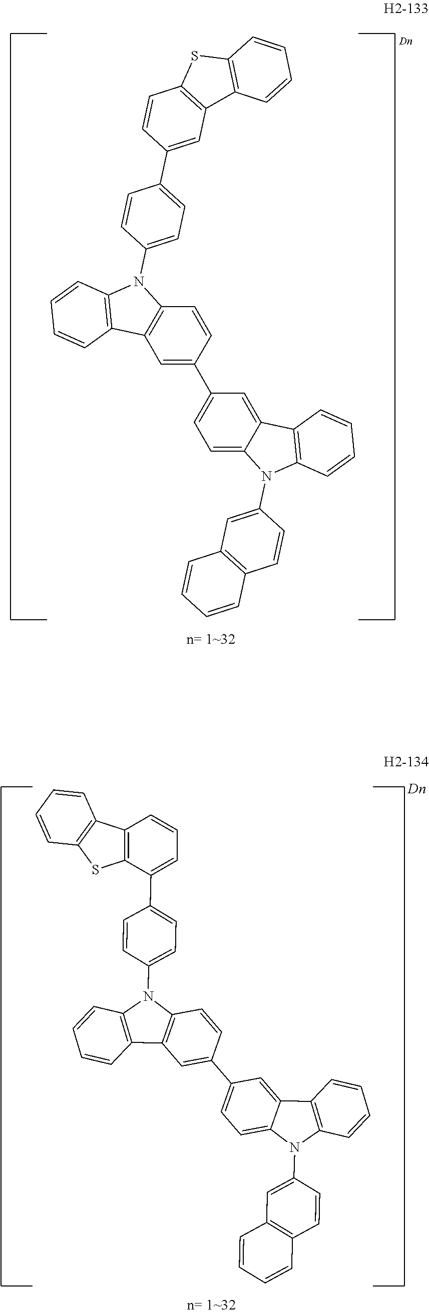

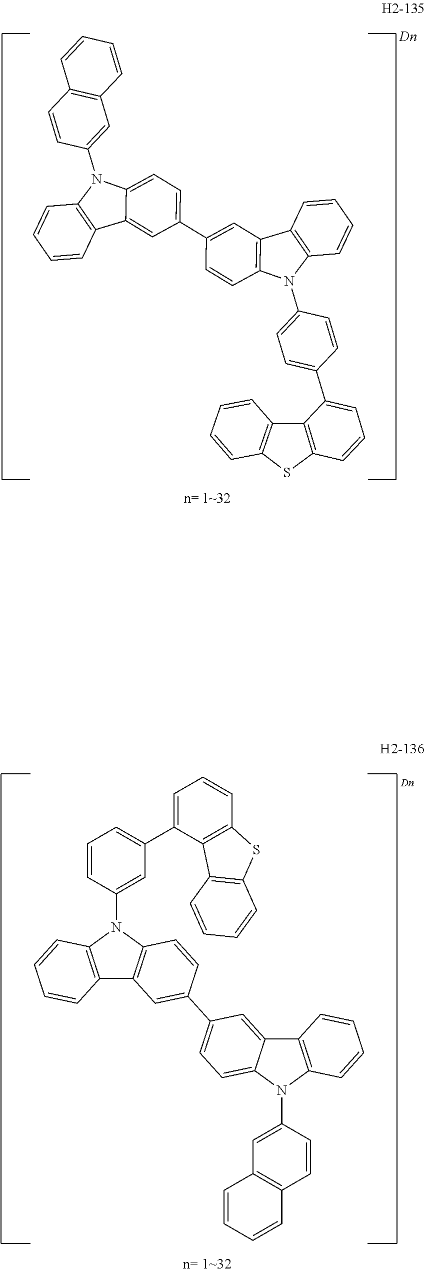

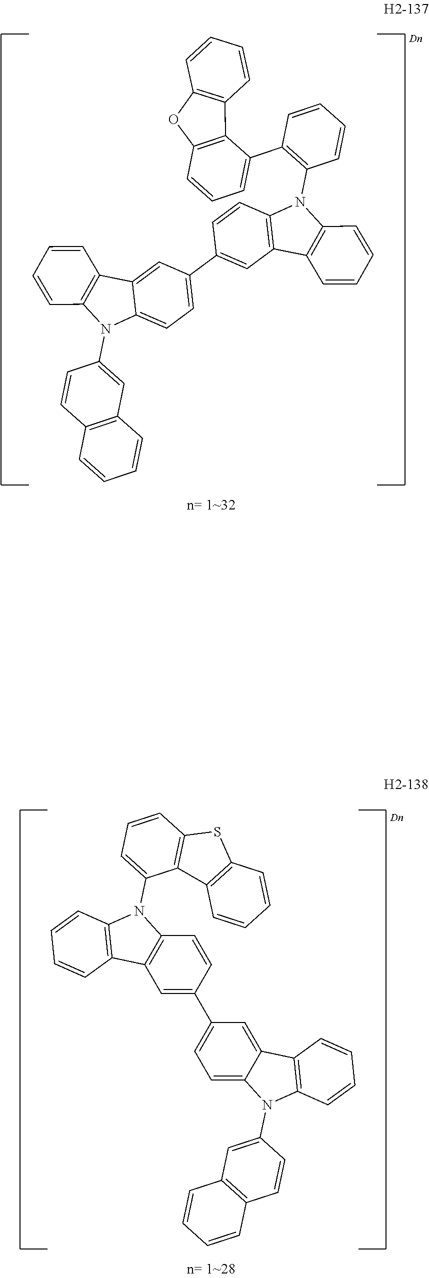

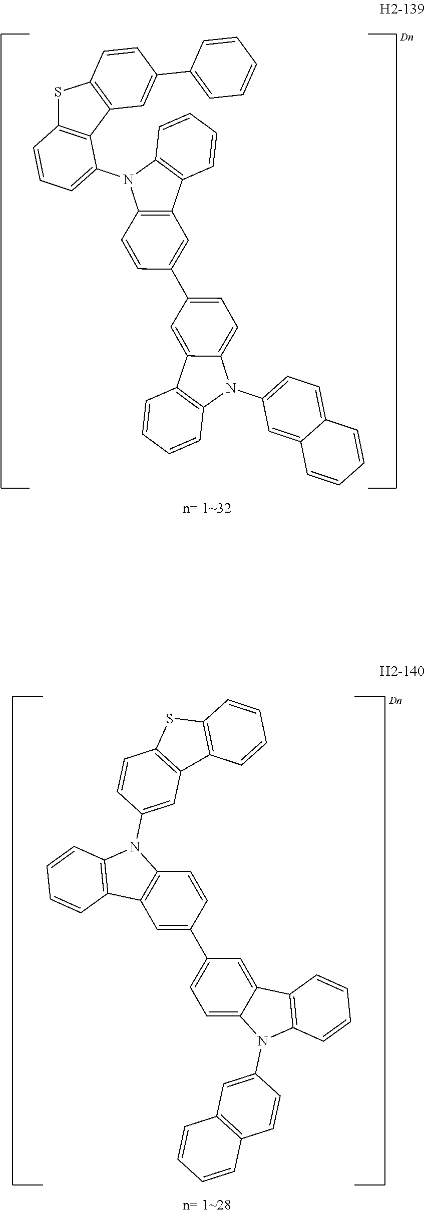

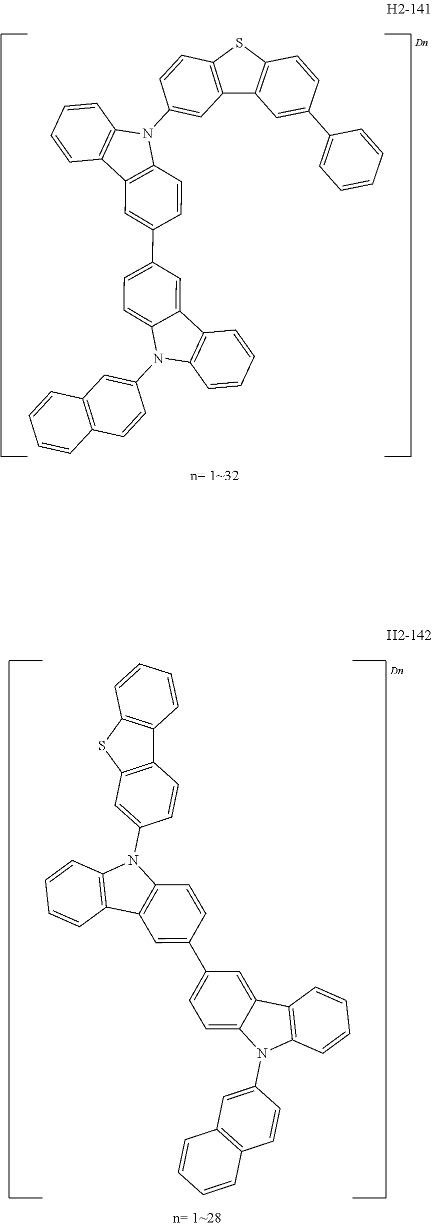

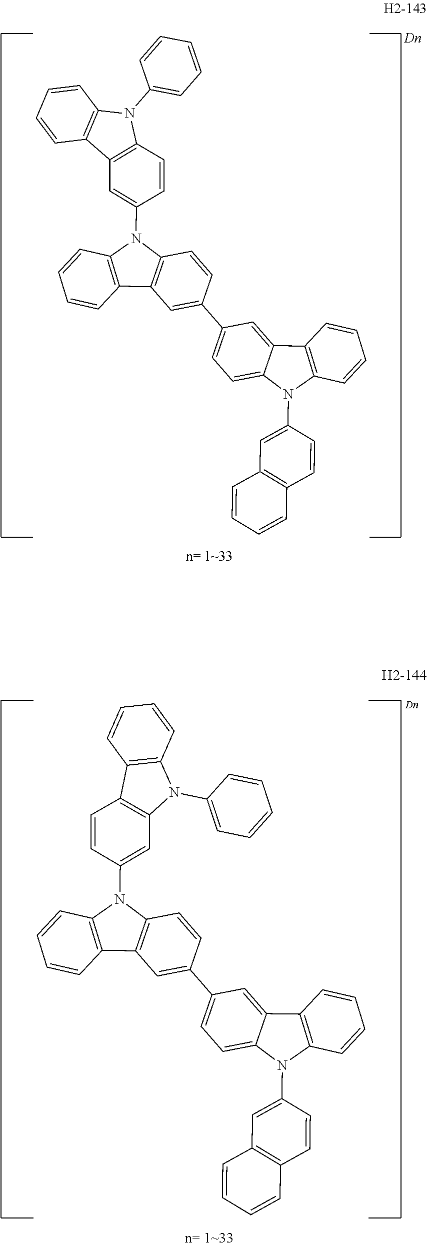

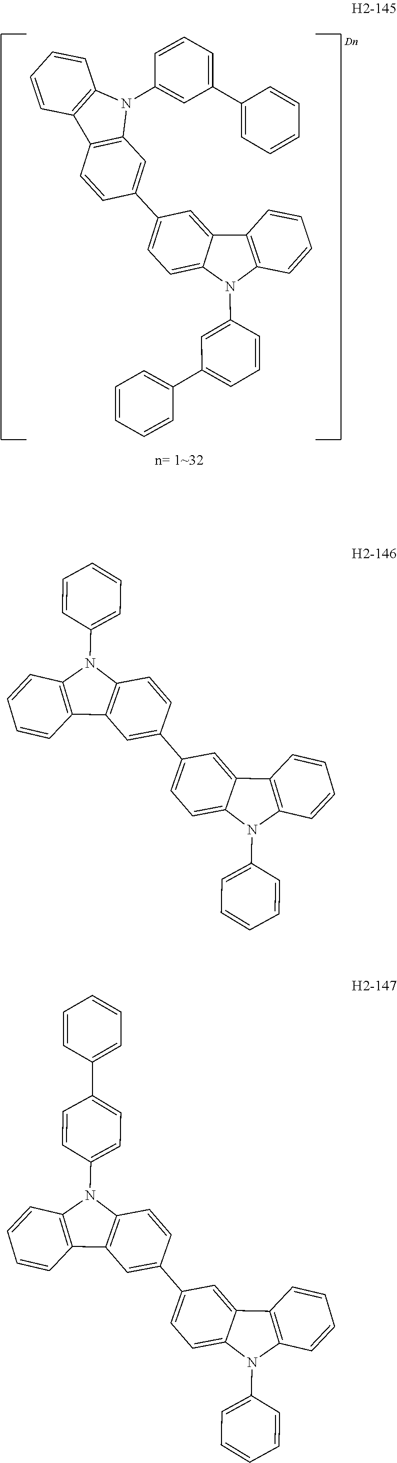

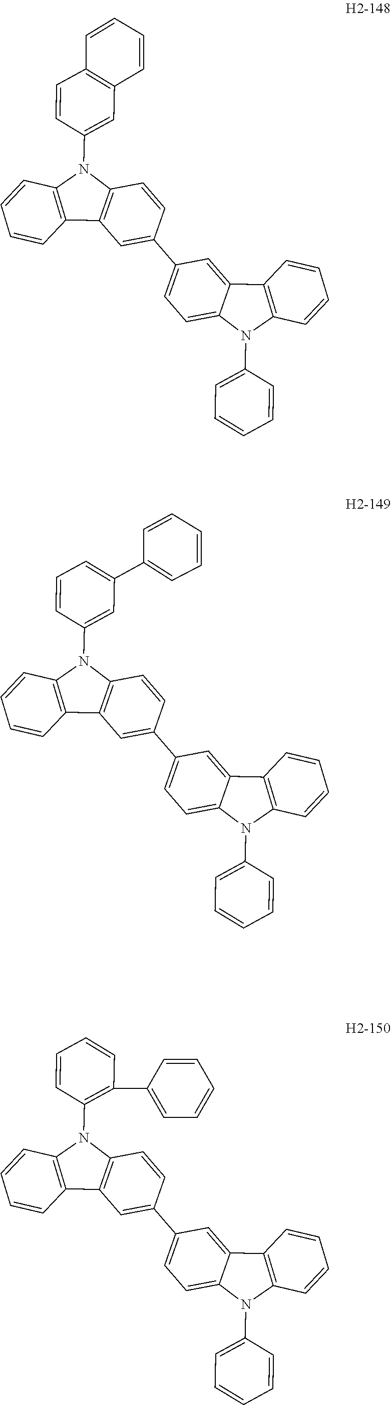 [SMILES: c1ccc(-n2c3ccccc3c3cc(-c4ccc5c(c4)c4ccccc4n5-c4ccc5ccccc5c4)ccc32)cc1 ; c1ccc(-c2cccc(-n3c4ccccc4c4cc(-c5ccc6c(c5)c5ccccc5n6-c5ccccc5)ccc43)c2)cc1 ; c1ccc(-c2ccccc2-n2c3ccccc3c3cc(-c4ccc5c(c4)c4ccccc4n5-c4ccccc4)ccc32)cc1]

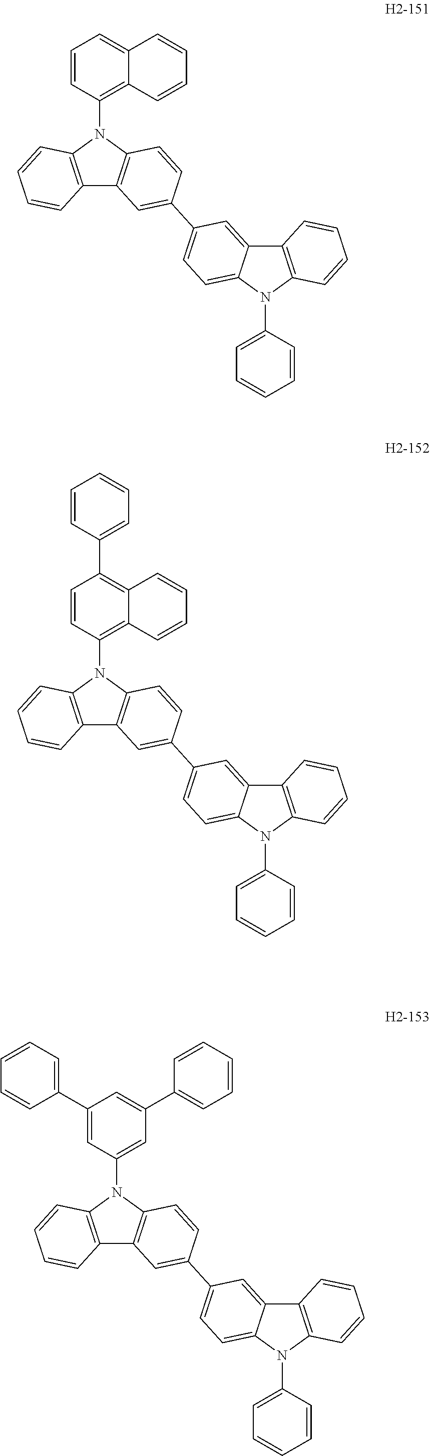 [SMILES: c1ccc(-n2c3ccccc3c3cc(-c4ccc5c(c4)c4ccccc4n5-c4cccc5ccccc45)ccc32)cc1 ; c1ccc(-c2ccc(-n3c4ccccc4c4cc(-c5ccc6c(c5)c5ccccc5n6-c5ccccc5)ccc43)c3ccccc23)cc1 ; c1ccc(-c2cc(-c3ccccc3)cc(-n3c4ccccc4c4cc(-c5ccc6c(c5)c5ccccc5n6-c5ccccc5)ccc43)c2)cc1]

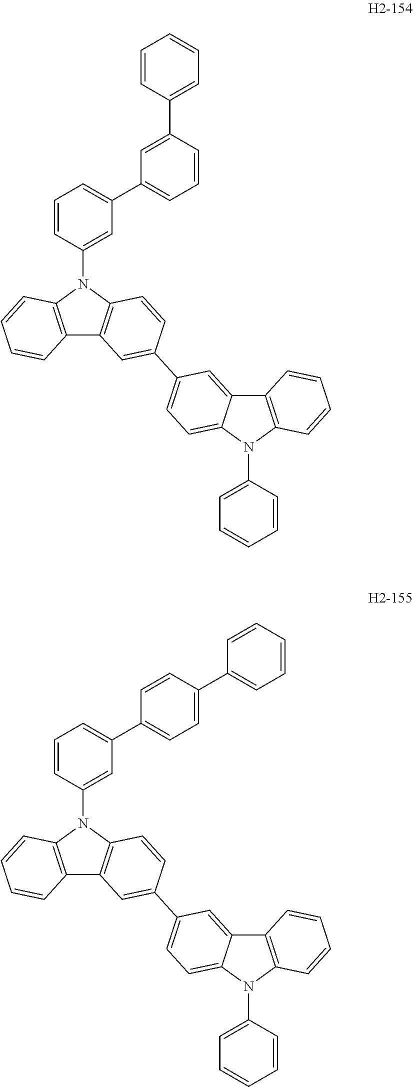 [SMILES: c1ccc(-c2cccc(-c3cccc(-n4c5ccccc5c5cc(-c6ccc7c(c6)c6ccccc6n7-c6ccccc6)ccc54)c3)c2)cc1 ; c1ccc(-c2ccc(-c3cccc(-n4c5ccccc5c5cc(-c6ccc7c(c6)c6ccccc6n7-c6ccccc6)ccc54)c3)cc2)cc1]

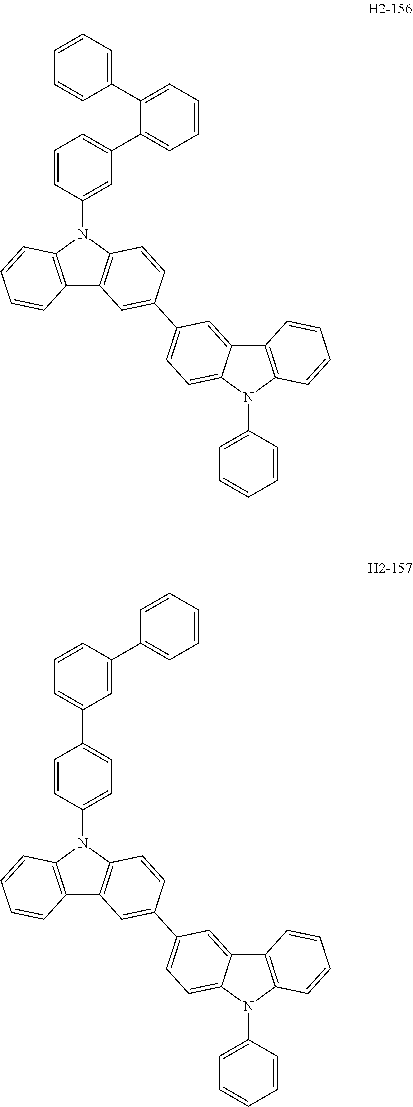 [SMILES: c1ccc(-c2ccccc2-c2cccc(-n3c4ccccc4c4cc(-c5ccc6c(c5)c5ccccc5n6-c5ccccc5)ccc43)c2)cc1 ; c1ccc(-c2cccc(-c3ccc(-n4c5ccccc5c5cc(-c6ccc7c(c6)c6ccccc6n7-c6ccccc6)ccc54)cc3)c2)cc1]

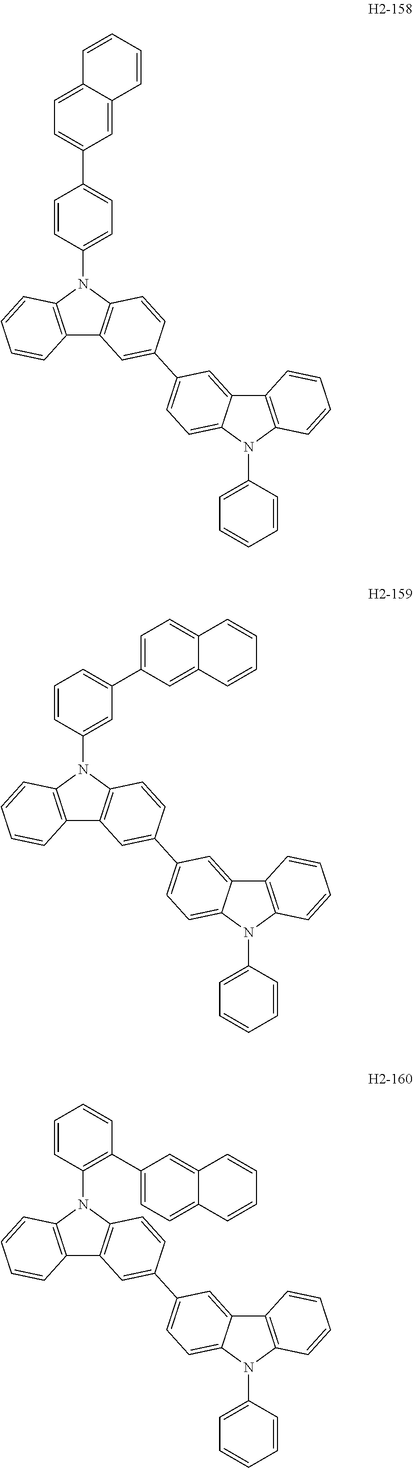 [SMILES: c1ccc(-n2c3ccccc3c3cc(-c4ccc5c(c4)c4ccccc4n5-c4ccc(-c5ccc6ccccc6c5)cc4)ccc32)cc1 ; c1ccc(-n2c3ccccc3c3cc(-c4ccc5c(c4)c4ccccc4n5-c4cccc(-c5ccc6ccccc6c5)c4)ccc32)cc1 ; c1ccc(-n2c3ccccc3c3cc(-c4ccc5c(c4)c4ccccc4n5-c4ccccc4-c4ccc5ccccc5c4)ccc32)cc1]

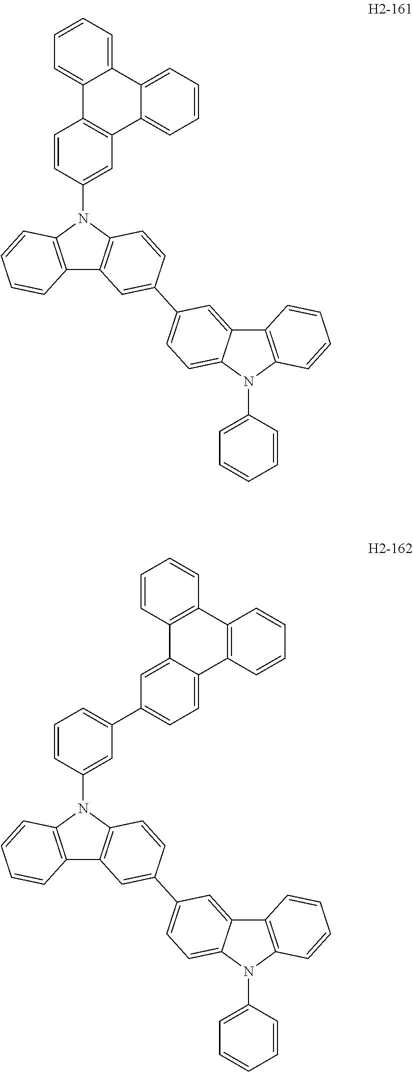 [SMILES: c1ccc(-n2c3ccccc3c3cc(-c4ccc5c(c4)c4ccccc4n5-c4ccc5c6ccccc6c6ccccc6c5c4)ccc32)cc1 ; c1ccc(-n2c3ccccc3c3cc(-c4ccc5c(c4)c4ccccc4n5-c4cccc(-c5ccc6c7ccccc7c7ccccc7c6c5)c4)ccc32)cc1]

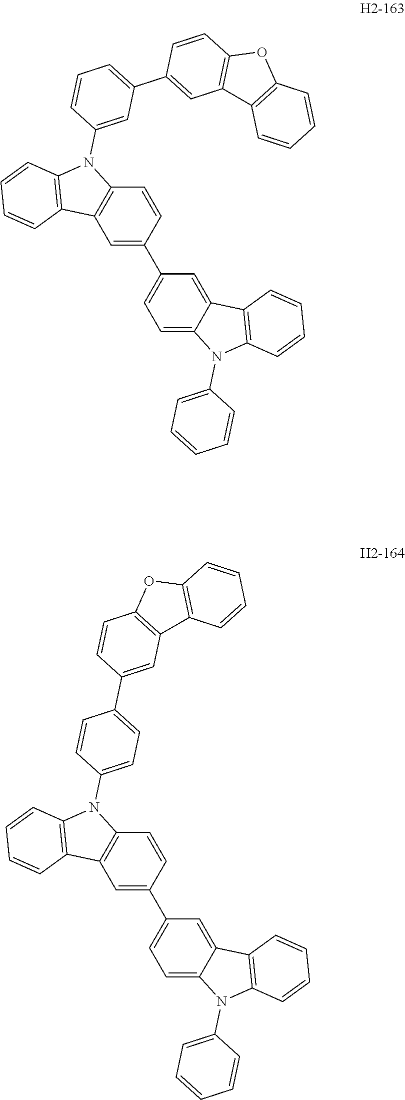 [SMILES: c1ccc(-n2c3ccccc3c3cc(-c4ccc5c(c4)c4ccccc4n5-c4cccc(-c5ccc6oc7ccccc7c6c5)c4)ccc32)cc1 ; c1ccc(-n2c3ccccc3c3cc(-c4ccc5c(c4)c4ccccc4n5-c4ccc(-c5ccc6oc7ccccc7c6c5)cc4)ccc32)cc1]

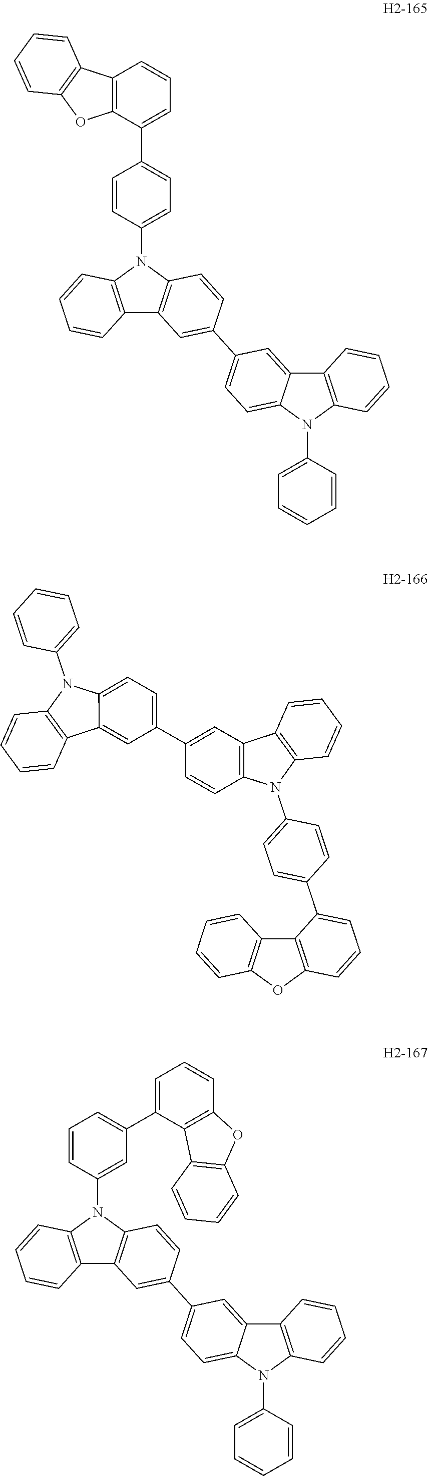 [SMILES: c1ccc(-n2c3ccccc3c3cc(-c4ccc5c(c4)c4ccccc4n5-c4ccc(-c5cccc6c5oc5ccccc56)cc4)ccc32)cc1 ; c1ccc(-n2c3ccccc3c3cc(-c4ccc5c(c4)c4ccccc4n5-c4ccc(-c5cccc6oc7ccccc7c56)cc4)ccc32)cc1 ; c1ccc(-n2c3ccccc3c3cc(-c4ccc5c(c4)c4ccccc4n5-c4cccc(-c5cccc6oc7ccccc7c56)c4)ccc32)cc1]

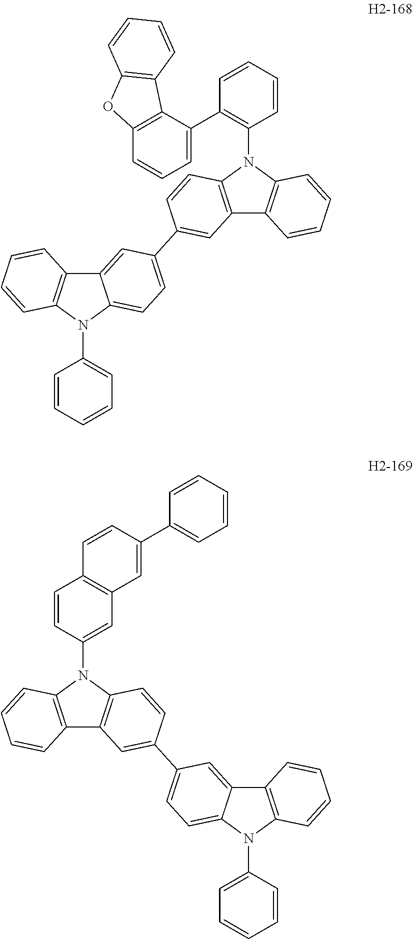 [SMILES: c1ccc(-n2c3ccccc3c3cc(-c4ccc5c(c4)c4ccccc4n5-c4ccccc4-c4cccc5oc6ccccc6c45)ccc32)cc1 ; c1ccc(-c2ccc3ccc(-n4c5ccccc5c5cc(-c6ccc7c(c6)c6ccccc6n7-c6ccccc6)ccc54)cc3c2)cc1]

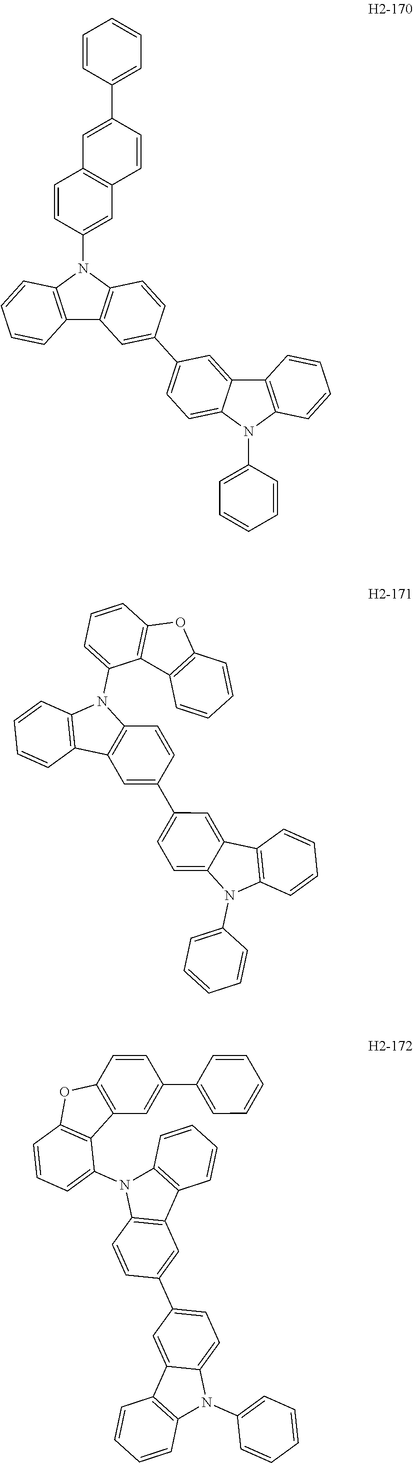 [SMILES: c1ccc(-c2ccc3cc(-n4c5ccccc5c5cc(-c6ccc7c(c6)c6ccccc6n7-c6ccccc6)ccc54)ccc3c2)cc1 ; c1ccc(-n2c3ccccc3c3cc(-c4ccc5c(c4)c4ccccc4n5-c4cccc5oc6ccccc6c45)ccc32)cc1 ; c1ccc(-c2ccc3oc4cccc(-n5c6ccccc6c6cc(-c7ccc8c(c7)c7ccccc7n8-c7ccccc7)ccc65)c4c3c2)cc1]

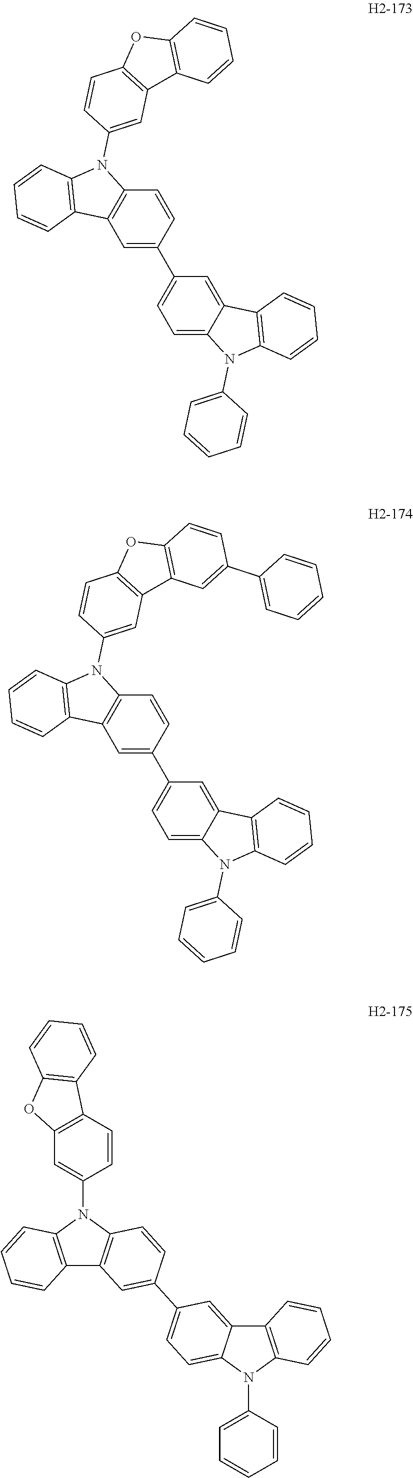 [SMILES: c1ccc(-n2c3ccccc3c3cc(-c4ccc5c(c4)c4ccccc4n5-c4ccc5oc6ccccc6c5c4)ccc32)cc1 ; c1ccc(-c2ccc3oc4ccc(-n5c6ccccc6c6cc(-c7ccc8c(c7)c7ccccc7n8-c7ccccc7)ccc65)cc4c3c2)cc1 ; c1ccc(-n2c3ccccc3c3cc(-c4ccc5c(c4)c4ccccc4n5-c4ccc5c(c4)oc4ccccc45)ccc32)cc1]

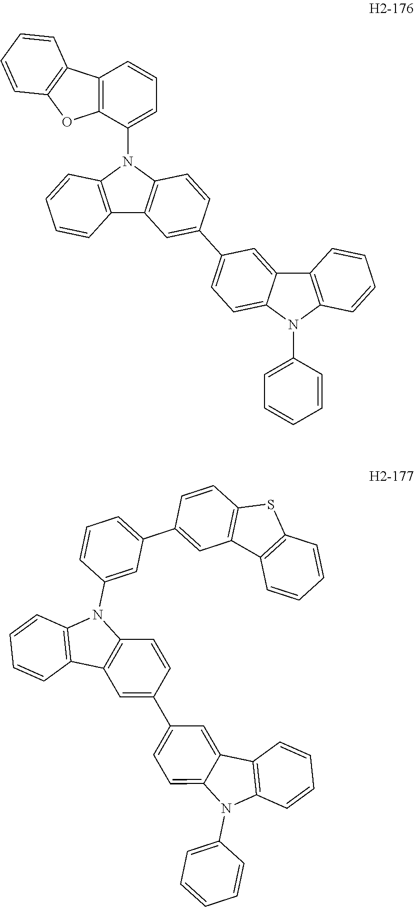 [SMILES: c1ccc(-n2c3ccccc3c3cc(-c4ccc5c(c4)c4ccccc4n5-c4cccc5c4oc4ccccc45)ccc32)cc1 ; c1ccc(-n2c3ccccc3c3cc(-c4ccc5c(c4)c4ccccc4n5-c4cccc(-c5ccc6sc7ccccc7c6c5)c4)ccc32)cc1]

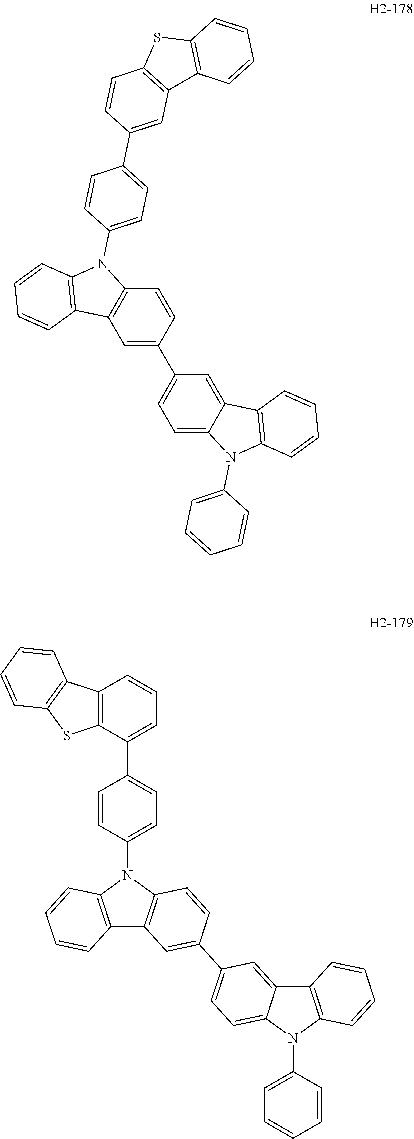 [SMILES: c1ccc(-n2c3ccccc3c3cc(-c4ccc5c(c4)c4ccccc4n5-c4ccc(-c5ccc6sc7ccccc7c6c5)cc4)ccc32)cc1 ; c1ccc(-n2c3ccccc3c3cc(-c4ccc5c(c4)c4ccccc4n5-c4ccc(-c5cccc6c5sc5ccccc56)cc4)ccc32)cc1]

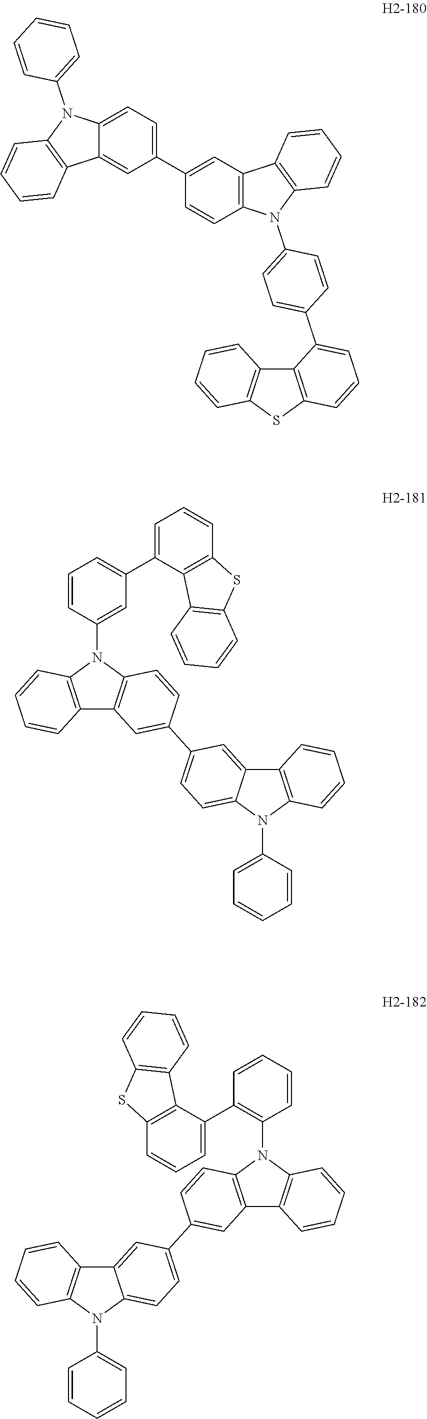 [SMILES: c1ccc(-n2c3ccccc3c3cc(-c4ccc5c(c4)c4ccccc4n5-c4ccc(-c5cccc6sc7ccccc7c56)cc4)ccc32)cc1 ; c1ccc(-n2c3ccccc3c3cc(-c4ccc5c(c4)c4ccccc4n5-c4cccc(-c5cccc6sc7ccccc7c56)c4)ccc32)cc1 ; c1ccc(-n2c3ccccc3c3cc(-c4ccc5c(c4)c4ccccc4n5-c4ccccc4-c4cccc5sc6ccccc6c45)ccc32)cc1]

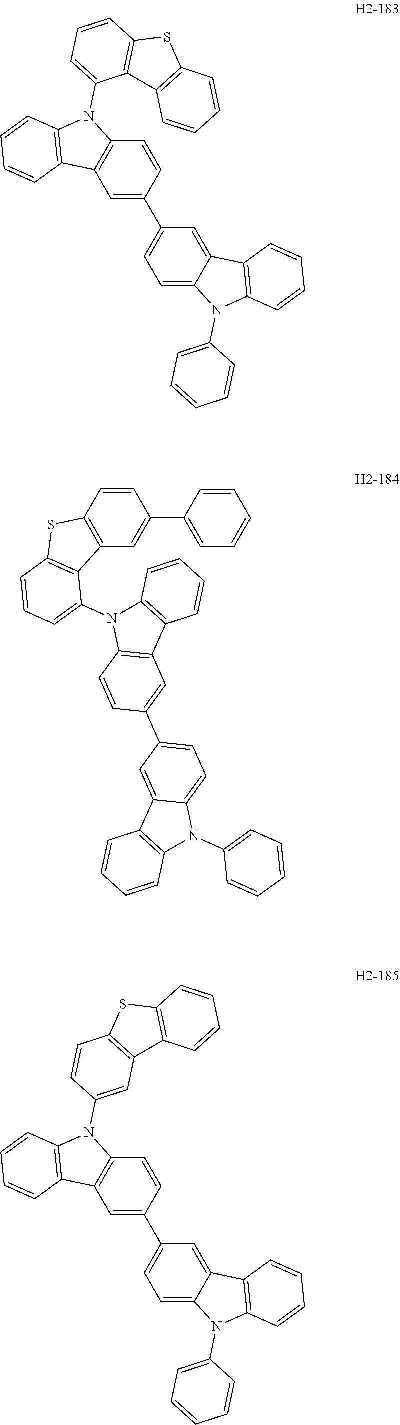 [SMILES: c1ccc(-n2c3ccccc3c3cc(-c4ccc5c(c4)c4ccccc4n5-c4cccc5sc6ccccc6c45)ccc32)cc1 ; c1ccc(-c2ccc3sc4cccc(-n5c6ccccc6c6cc(-c7ccc8c(c7)c7ccccc7n8-c7ccccc7)ccc65)c4c3c2)cc1 ; c1ccc(-n2c3ccccc3c3cc(-c4ccc5c(c4)c4ccccc4n5-c4ccc5sc6ccccc6c5c4)ccc32)cc1]

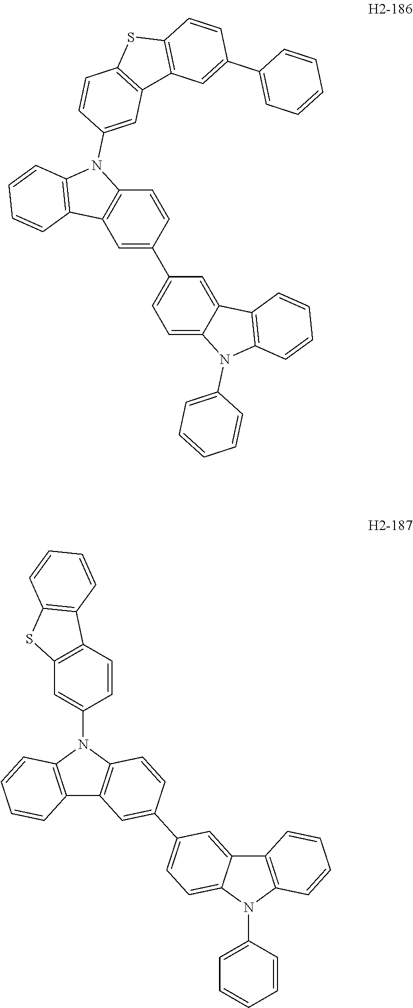 [SMILES: c1ccc(-c2ccc3sc4ccc(-n5c6ccccc6c6cc(-c7ccc8c(c7)c7ccccc7n8-c7ccccc7)ccc65)cc4c3c2)cc1 ; c1ccc(-n2c3ccccc3c3cc(-c4ccc5c(c4)c4ccccc4n5-c4ccc5c(c4)sc4ccccc45)ccc32)cc1]

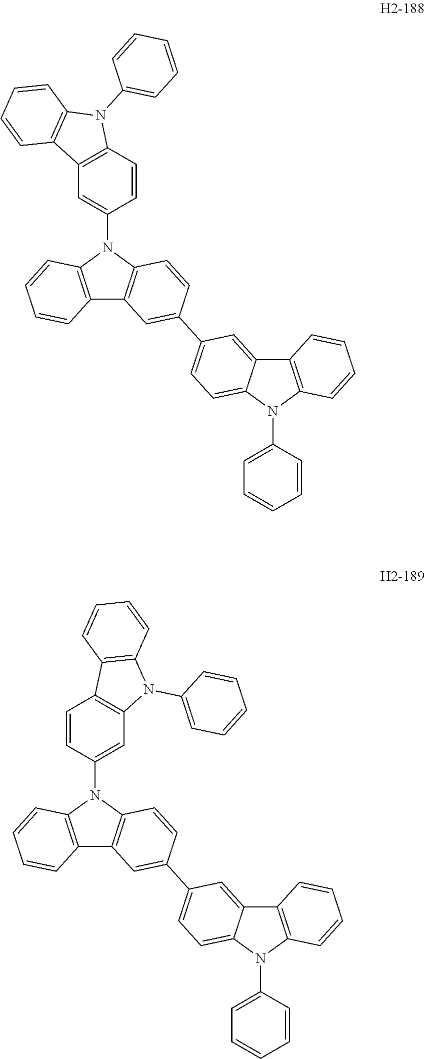 [SMILES: c1ccc(-n2c3ccccc3c3cc(-c4ccc5c(c4)c4ccccc4n5-c4ccc5c(c4)c4ccccc4n5-c4ccccc4)ccc32)cc1 ; c1ccc(-n2c3ccccc3c3cc(-c4ccc5c(c4)c4ccccc4n5-c4ccc5c6ccccc6n(-c6ccccc6)c5c4)ccc32)cc1]

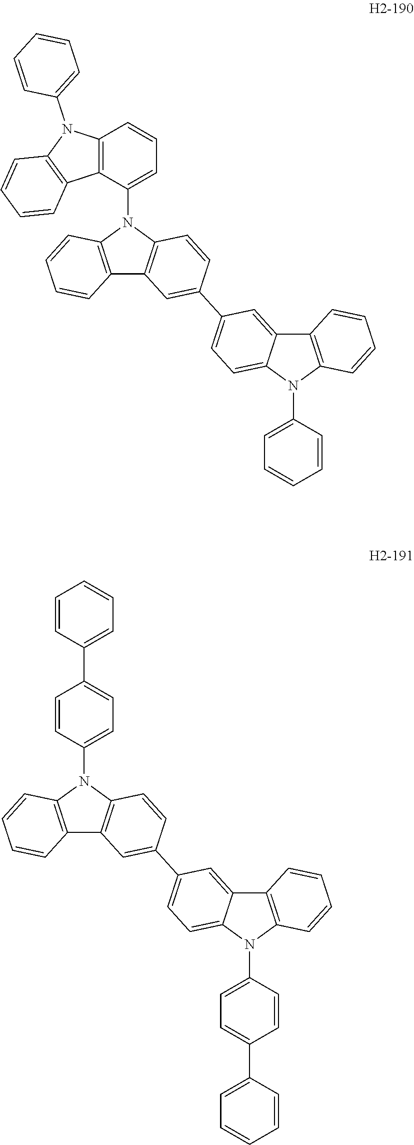 [SMILES: c1ccc(-n2c3ccccc3c3cc(-c4ccc5c(c4)c4ccccc4n5-c4cccc5c4c4ccccc4n5-c4ccccc4)ccc32)cc1 ; c1ccc(-c2ccc(-n3c4ccccc4c4cc(-c5ccc6c(c5)c5ccccc5n6-c5ccc(-c6ccccc6)cc5)ccc43)cc2)cc1]

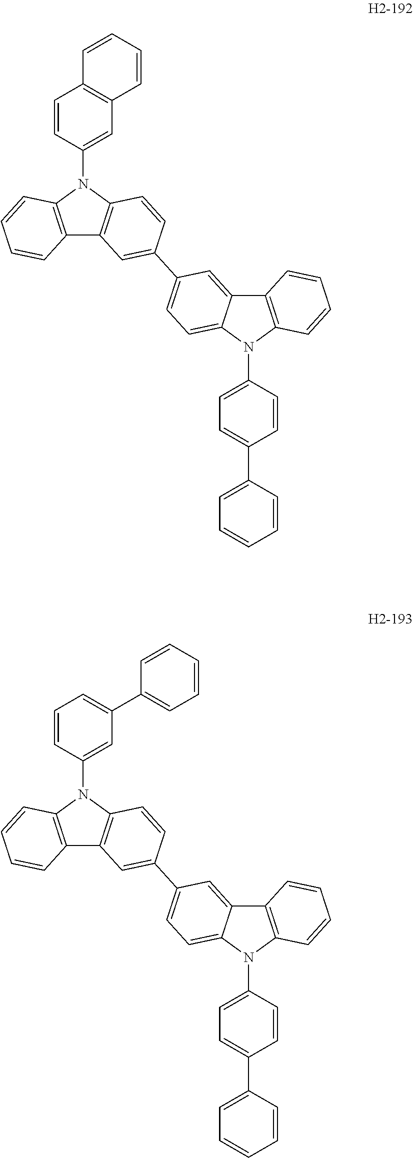 [SMILES: c1ccc(-c2ccc(-n3c4ccccc4c4cc(-c5ccc6c(c5)c5ccccc5n6-c5ccc6ccccc6c5)ccc43)cc2)cc1 ; c1ccc(-c2ccc(-n3c4ccccc4c4cc(-c5ccc6c(c5)c5ccccc5n6-c5cccc(-c6ccccc6)c5)ccc43)cc2)cc1]

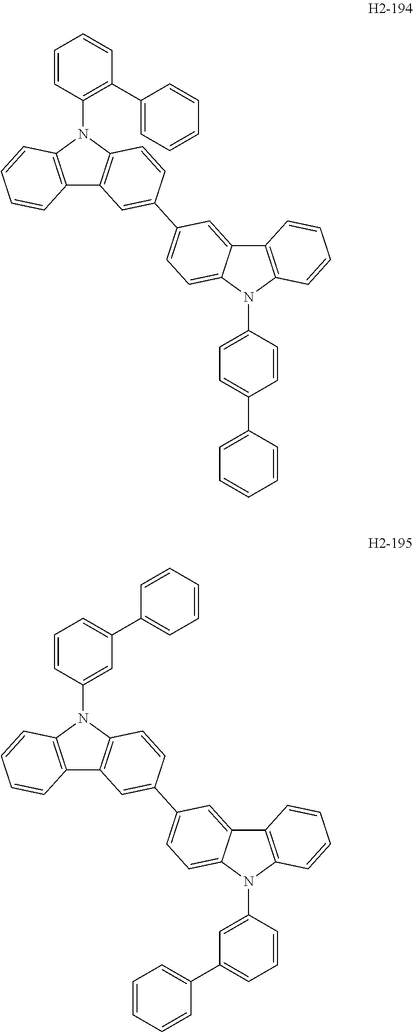 [SMILES: c1ccc(-c2ccc(-n3c4ccccc4c4cc(-c5ccc6c(c5)c5ccccc5n6-c5ccccc5-c5ccccc5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1]

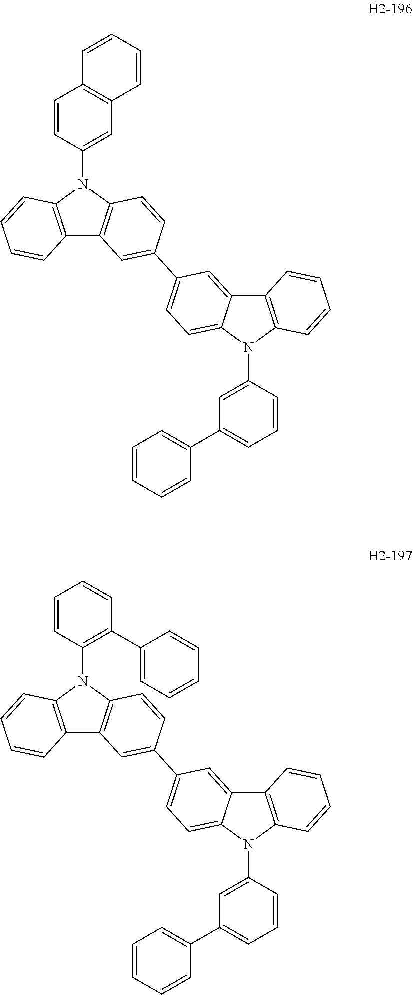 [SMILES: c1ccc(-c2cccc(-n3c4ccccc4c4cc(-c5ccc6c(c5)c5ccccc5n6-c5ccc6ccccc6c5)ccc43)c2)cc1 ; c1ccc(-c2cccc(-n3c4ccccc4c4cc(-c5ccc6c(c5)c5ccccc5n6-c5ccccc5-c5ccccc5)ccc43)c2)cc1]

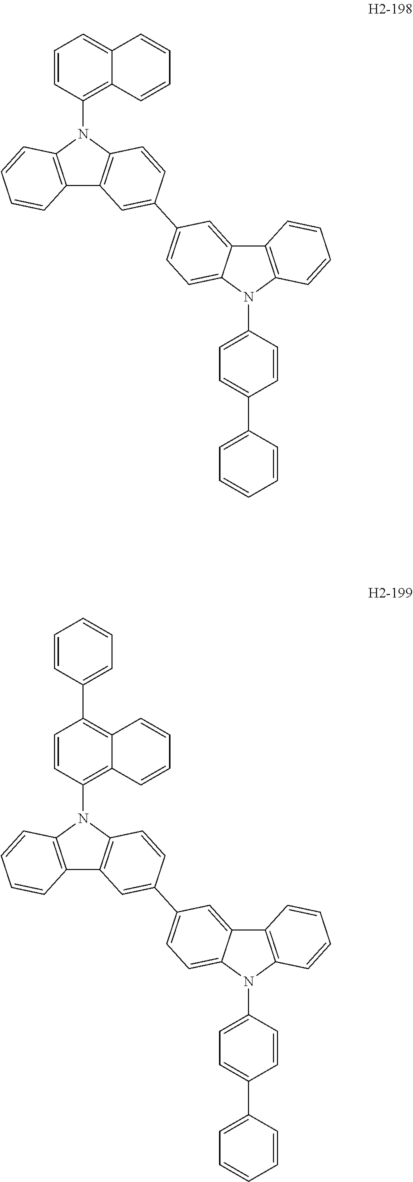 [SMILES: c1ccc(-c2ccc(-n3c4ccccc4c4cc(-c5ccc6c(c5)c5ccccc5n6-c5cccc6ccccc56)ccc43)cc2)cc1 ; c1ccc(-c2ccc(-n3c4ccccc4c4cc(-c5ccc6c(c5)c5ccccc5n6-c5ccc(-c6ccccc6)c6ccccc56)ccc43)cc2)cc1]

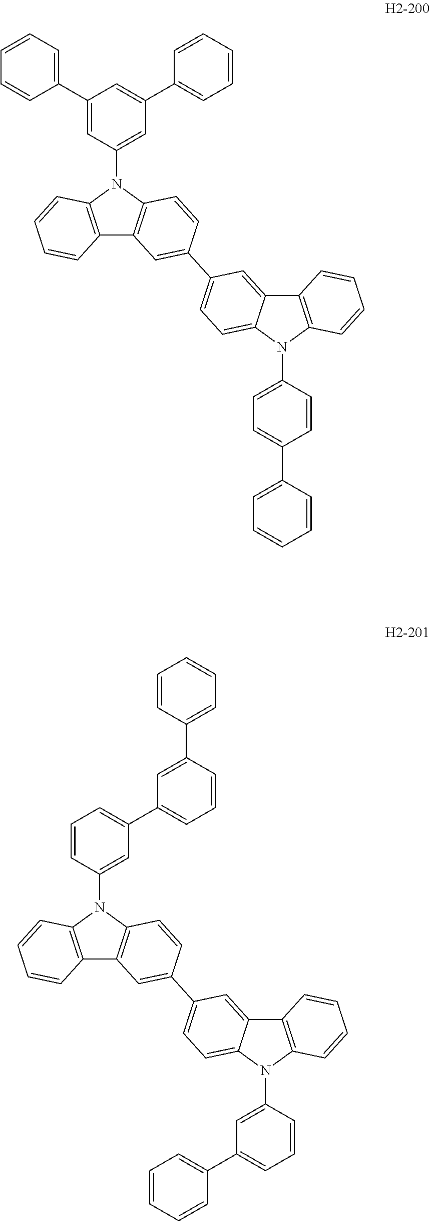 [SMILES: c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1 ; c1ccc(-c2cccc(-c3cccc(-n4c5ccccc5c5cc(-c6ccc7c(c6)c6ccccc6n7-c6cccc(-c7ccccc7)c6)ccc54)c3)c2)cc1]

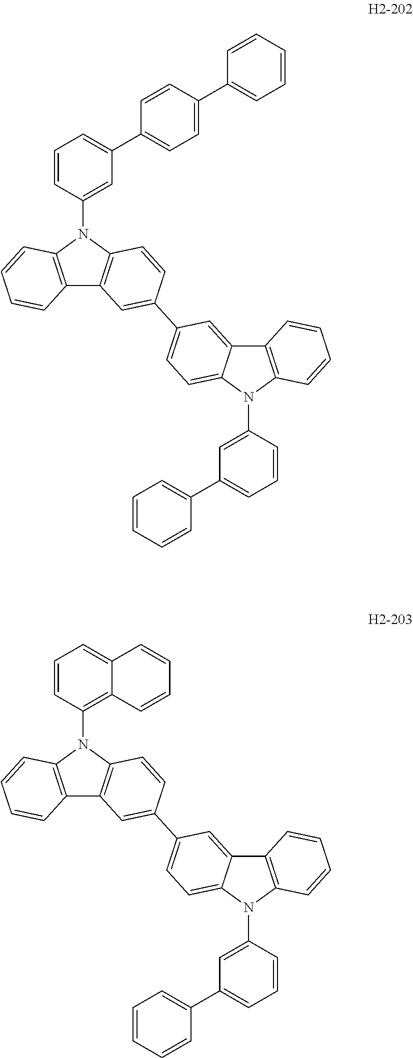 [SMILES: c1ccc(-c2ccc(-c3cccc(-n4c5ccccc5c5cc(-c6ccc7c(c6)c6ccccc6n7-c6cccc(-c7ccccc7)c6)ccc54)c3)cc2)cc1 ; c1ccc(-c2cccc(-n3c4ccccc4c4cc(-c5ccc6c(c5)c5ccccc5n6-c5cccc6ccccc56)ccc43)c2)cc1]

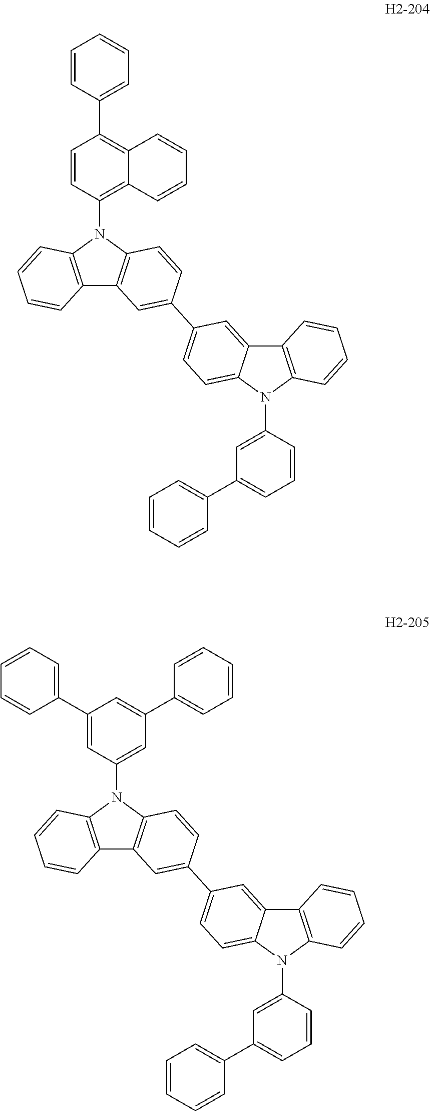 [SMILES: c1ccc(-c2cccc(-n3c4ccccc4c4cc(-c5ccc6c(c5)c5ccccc5n6-c5ccc(-c6ccccc6)c6ccccc56)ccc43)c2)cc1 ; c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1]

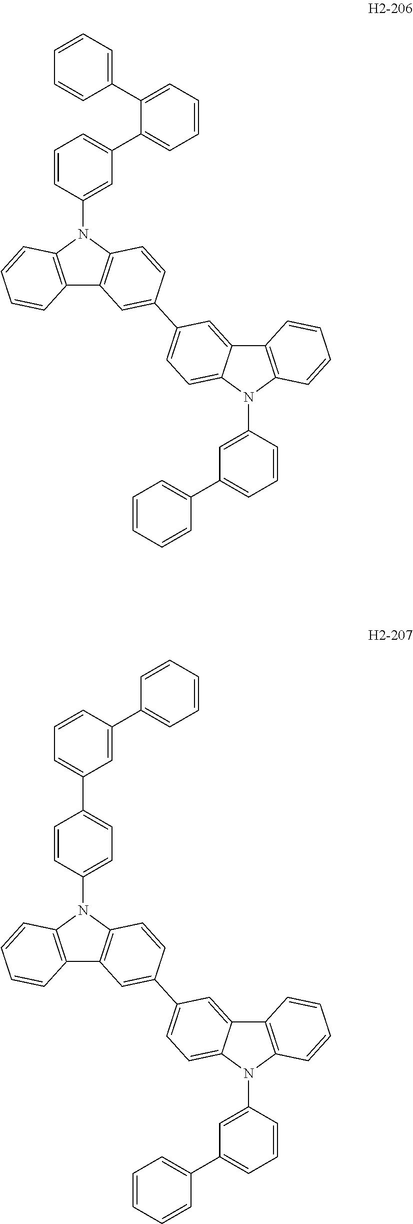 [SMILES: c1ccc(-c2cccc(-n3c4ccccc4c4cc(-c5ccc6c(c5)c5ccccc5n6-c5cccc(-c6ccccc6-c6ccccc6)c5)ccc43)c2)cc1 ; c1ccc(-c2cccc(-c3ccc(-n4c5ccccc5c5cc(-c6ccc7c(c6)c6ccccc6n7-c6cccc(-c7ccccc7)c6)ccc54)cc3)c2)cc1]

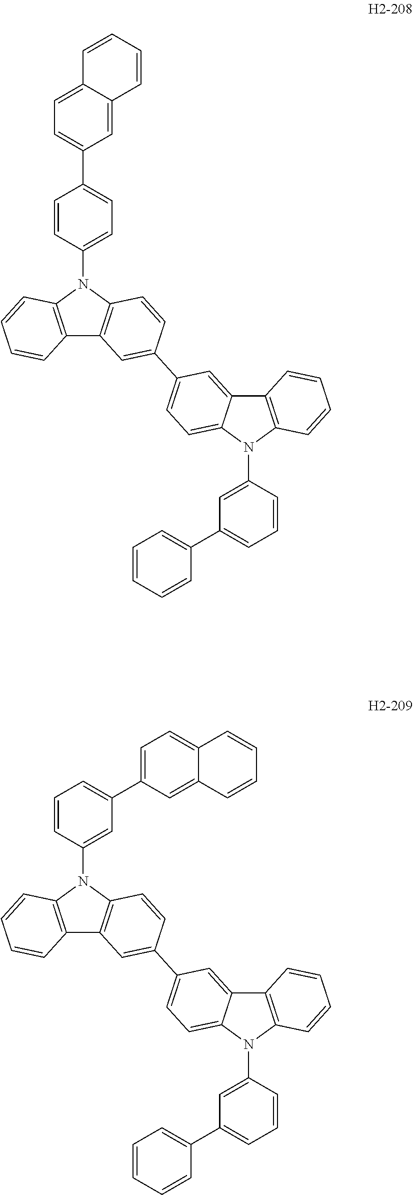 [SMILES: c1ccc(-c2cccc(-n3c4ccccc4c4cc(-c5ccc6c(c5)c5ccccc5n6-c5ccc(-c6ccc7ccccc7c6)cc5)ccc43)c2)cc1 ; c1ccc(-c2cccc(-n3c4ccccc4c4cc(-c5ccc6c(c5)c5ccccc5n6-c5cccc(-c6ccc7ccccc7c6)c5)ccc43)c2)cc1]

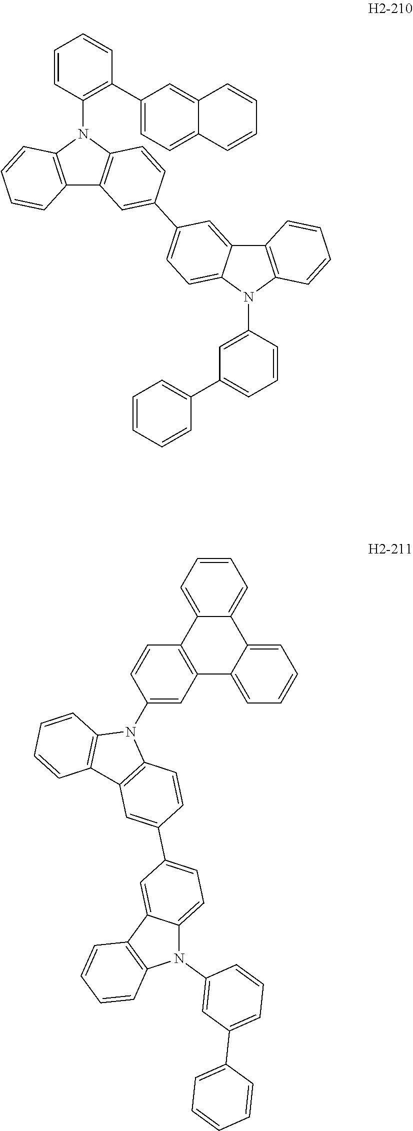 [SMILES: c1ccc(-c2cccc(-n3c4ccccc4c4cc(-c5ccc6c(c5)c5ccccc5n6-c5ccccc5-c5ccc6ccccc6c5)ccc43)c2)cc1 ; c1ccc(-c2cccc(-n3c4ccccc4c4cc(-c5ccc6c(c5)c5ccccc5n6-c5ccc6c7ccccc7c7ccccc7c6c5)ccc43)c2)cc1]

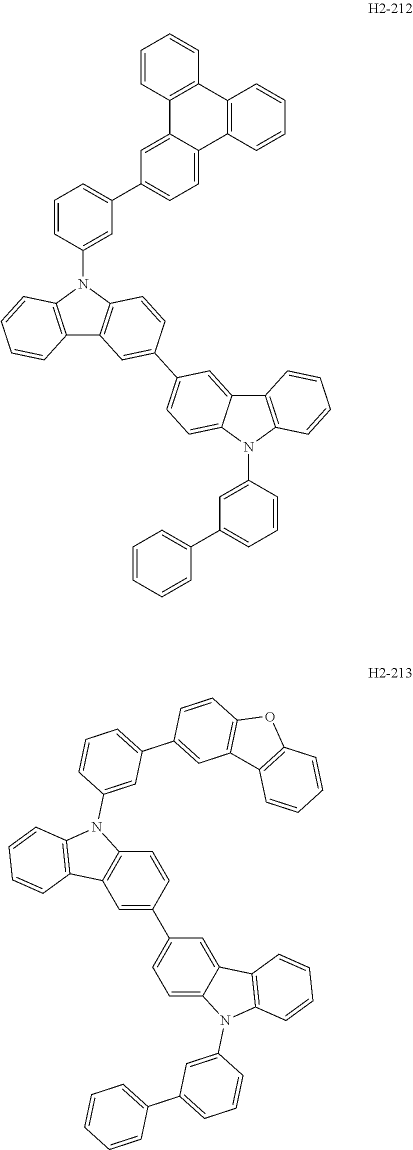 [SMILES: c1ccc(-c2cccc(-n3c4ccccc4c4cc(-c5ccc6c(c5)c5ccccc5n6-c5cccc(-c6ccc7c8ccccc8c8ccccc8c7c6)c5)ccc43)c2)cc1 ; c1ccc(-c2cccc(-n3c4ccccc4c4cc(-c5ccc6c(c5)c5ccccc5n6-c5cccc(-c6ccc7oc8ccccc8c7c6)c5)ccc43)c2)cc1]

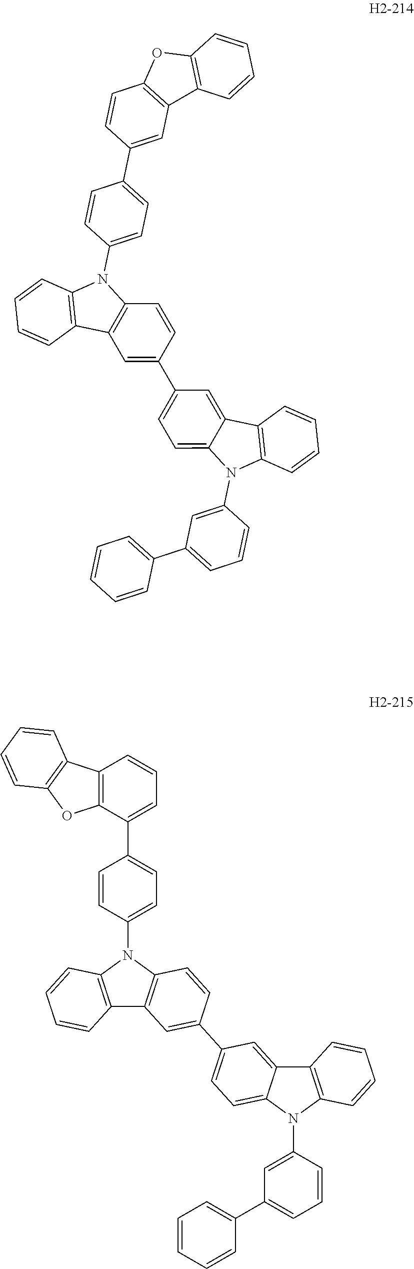 [SMILES: c1ccc(-c2cccc(-n3c4ccccc4c4cc(-c5ccc6c(c5)c5ccccc5n6-c5ccc(-c6ccc7oc8ccccc8c7c6)cc5)ccc43)c2)cc1 ; c1ccc(-c2cccc(-n3c4ccccc4c4cc(-c5ccc6c(c5)c5ccccc5n6-c5ccc(-c6cccc7c6oc6ccccc67)cc5)ccc43)c2)cc1]

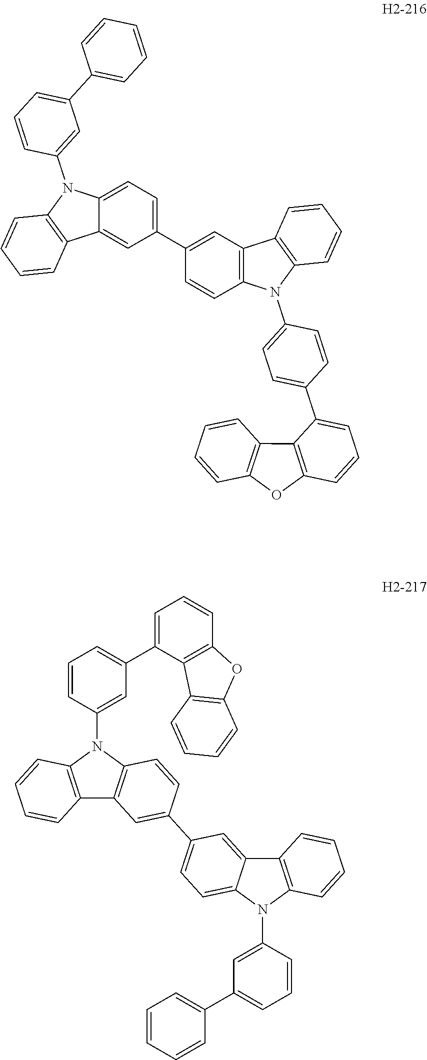 [SMILES: c1ccc(-c2cccc(-n3c4ccccc4c4cc(-c5ccc6c(c5)c5ccccc5n6-c5ccc(-c6cccc7oc8ccccc8c67)cc5)ccc43)c2)cc1 ; c1ccc(-c2cccc(-n3c4ccccc4c4cc(-c5ccc6c(c5)c5ccccc5n6-c5cccc(-c6cccc7oc8ccccc8c67)c5)ccc43)c2)cc1]

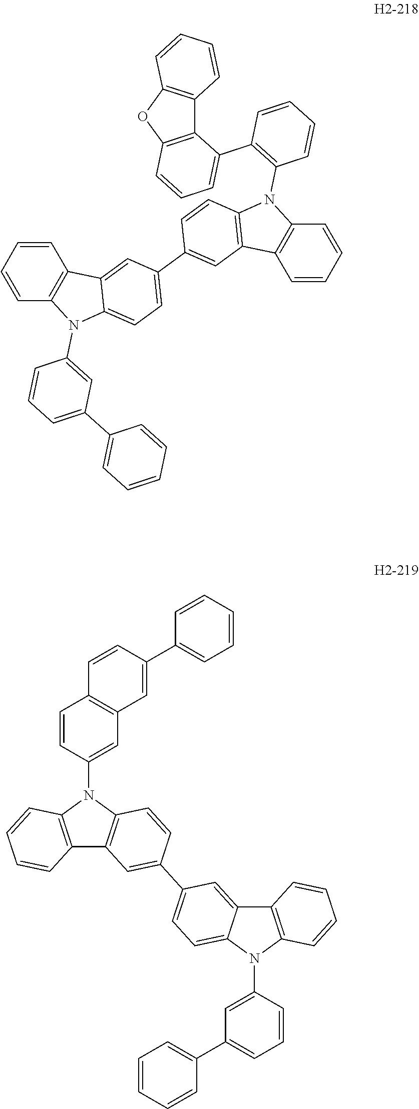 [SMILES: c1ccc(-c2cccc(-n3c4ccccc4c4cc(-c5ccc6c(c5)c5ccccc5n6-c5ccccc5-c5cccc6oc7ccccc7c56)ccc43)c2)cc1 ; c1ccc(-c2cccc(-n3c4ccccc4c4cc(-c5ccc6c(c5)c5ccccc5n6-c5ccc6ccc(-c7ccccc7)cc6c5)ccc43)c2)cc1]

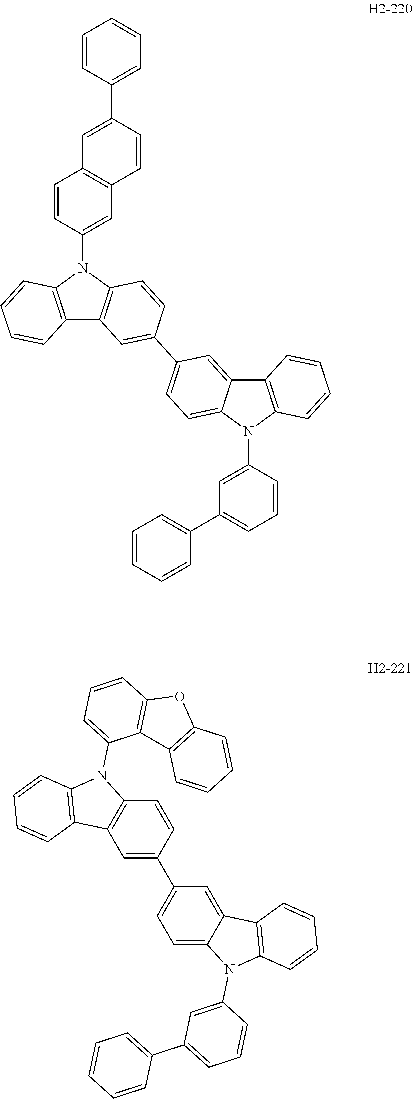 [SMILES: c1ccc(-c2cccc(-n3c4ccccc4c4cc(-c5ccc6c(c5)c5ccccc5n6-c5ccc6cc(-c7ccccc7)ccc6c5)ccc43)c2)cc1 ; c1ccc(-c2cccc(-n3c4ccccc4c4cc(-c5ccc6c(c5)c5ccccc5n6-c5cccc6oc7ccccc7c56)ccc43)c2)cc1]

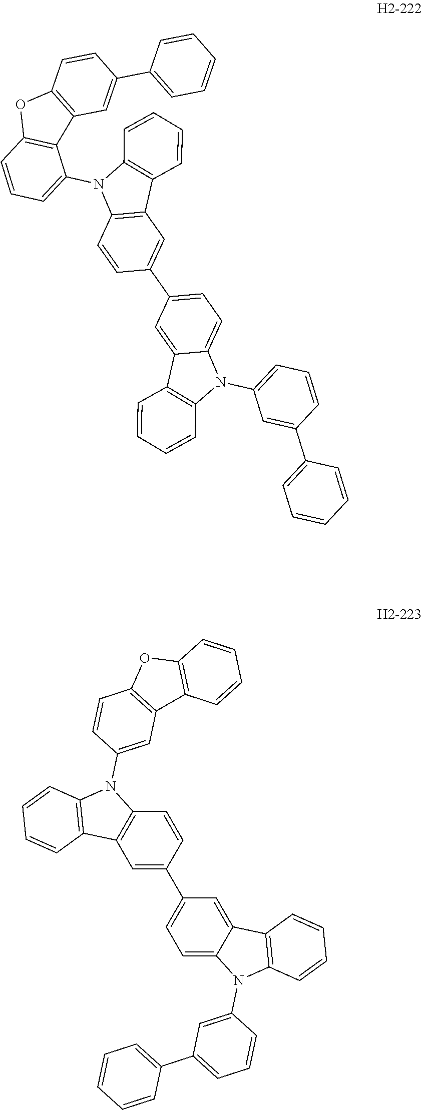 [SMILES: c1ccc(-c2cccc(-n3c4ccccc4c4cc(-c5ccc6c(c5)c5ccccc5n6-c5cccc6oc7ccc(-c8ccccc8)cc7c56)ccc43)c2)cc1 ; c1ccc(-c2cccc(-n3c4ccccc4c4cc(-c5ccc6c(c5)c5ccccc5n6-c5ccc6oc7ccccc7c6c5)ccc43)c2)cc1]

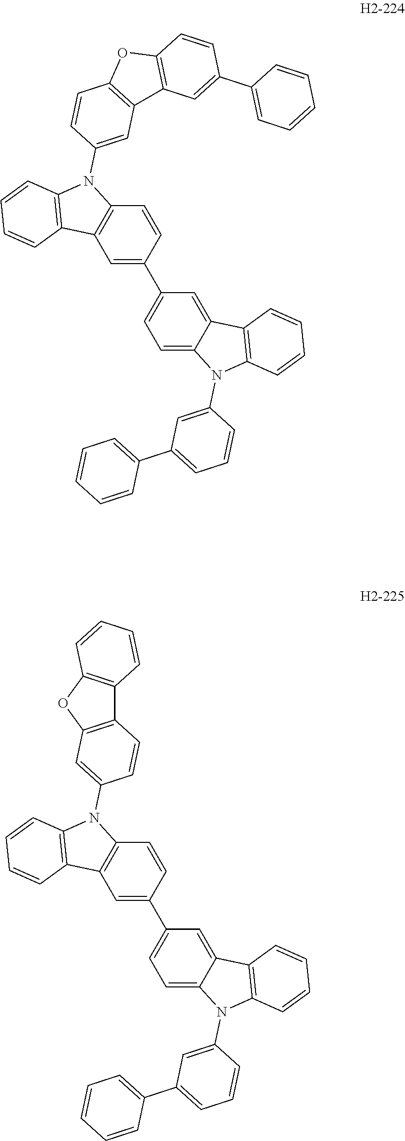 [SMILES: c1ccc(-c2cccc(-n3c4ccccc4c4cc(-c5ccc6c(c5)c5ccccc5n6-c5ccc6oc7ccc(-c8ccccc8)cc7c6c5)ccc43)c2)cc1 ; c1ccc(-c2cccc(-n3c4ccccc4c4cc(-c5ccc6c(c5)c5ccccc5n6-c5ccc6c(c5)oc5ccccc56)ccc43)c2)cc1]

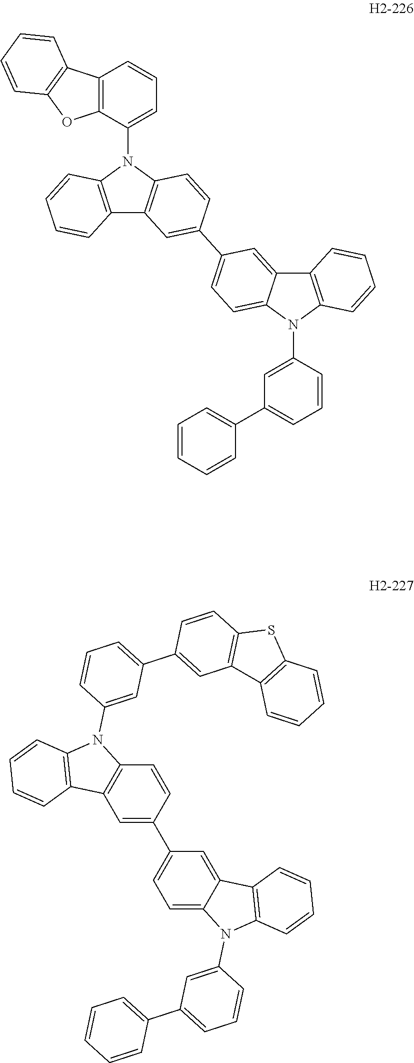 [SMILES: c1ccc(-c2cccc(-n3c4ccccc4c4cc(-c5ccc6c(c5)c5ccccc5n6-c5cccc6c5oc5ccccc56)ccc43)c2)cc1 ; c1ccc(-c2cccc(-n3c4ccccc4c4cc(-c5ccc6c(c5)c5ccccc5n6-c5cccc(-c6ccc7sc8ccccc8c7c6)c5)ccc43)c2)cc1]

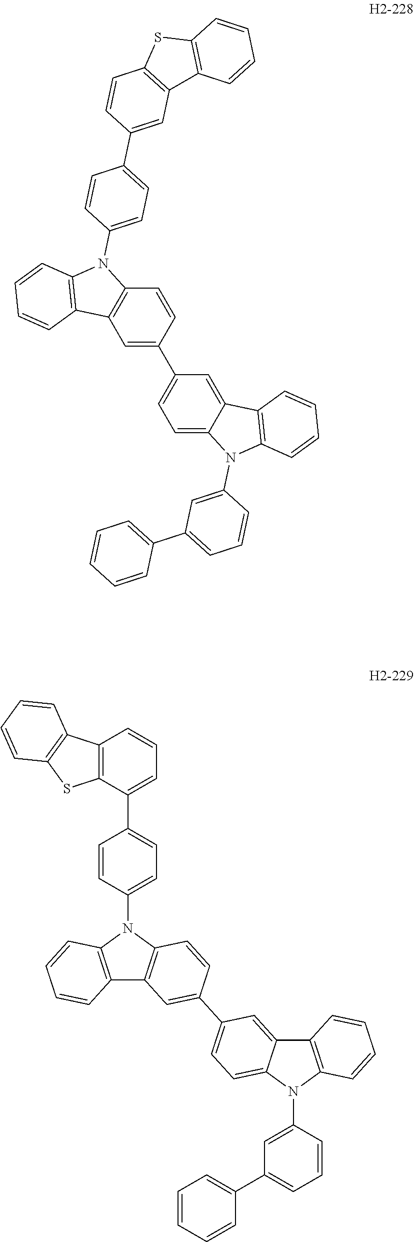 [SMILES: c1ccc(-c2cccc(-n3c4ccccc4c4cc(-c5ccc6c(c5)c5ccccc5n6-c5ccc(-c6ccc7sc8ccccc8c7c6)cc5)ccc43)c2)cc1 ; c1ccc(-c2cccc(-n3c4ccccc4c4cc(-c5ccc6c(c5)c5ccccc5n6-c5ccc(-c6cccc7c6sc6ccccc67)cc5)ccc43)c2)cc1]

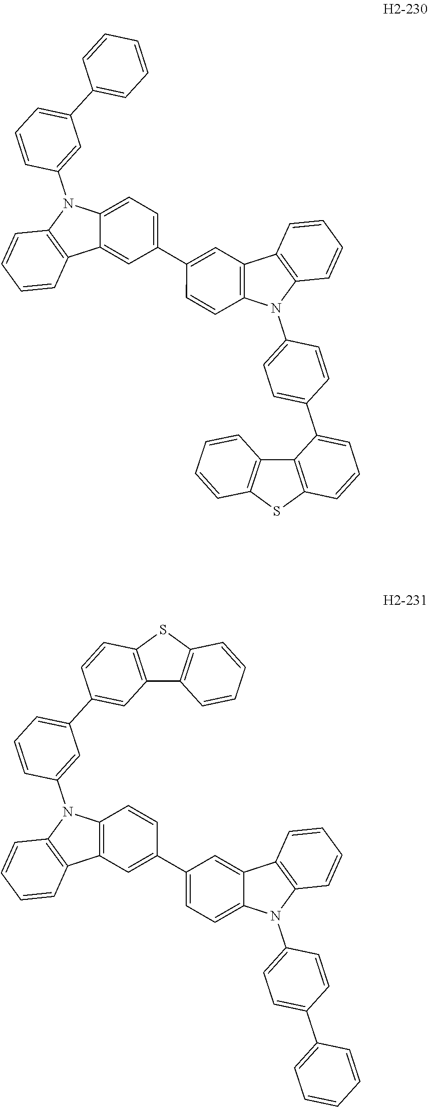 [SMILES: c1ccc(-c2cccc(-n3c4ccccc4c4cc(-c5ccc6c(c5)c5ccccc5n6-c5ccc(-c6cccc7sc8ccccc8c67)cc5)ccc43)c2)cc1 ; c1ccc(-c2ccc(-n3c4ccccc4c4cc(-c5ccc6c(c5)c5ccccc5n6-c5cccc(-c6ccc7sc8ccccc8c7c6)c5)ccc43)cc2)cc1]

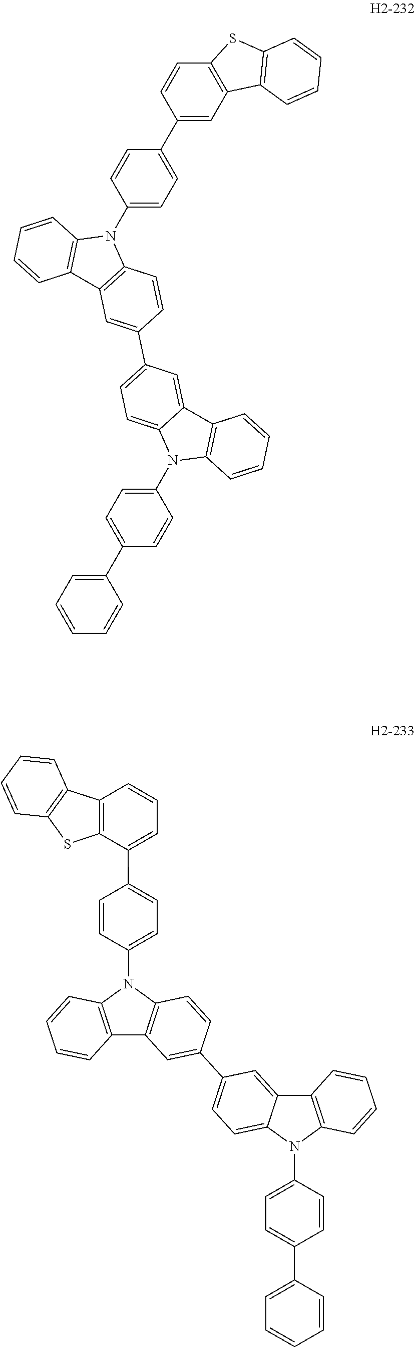 [SMILES: c1ccc(-c2ccc(-n3c4ccccc4c4cc(-c5ccc6c(c5)c5ccccc5n6-c5ccc(-c6ccc7sc8ccccc8c7c6)cc5)ccc43)cc2)cc1 ; c1ccc(-c2ccc(-n3c4ccccc4c4cc(-c5ccc6c(c5)c5ccccc5n6-c5ccc(-c6cccc7c6sc6ccccc67)cc5)ccc43)cc2)cc1]

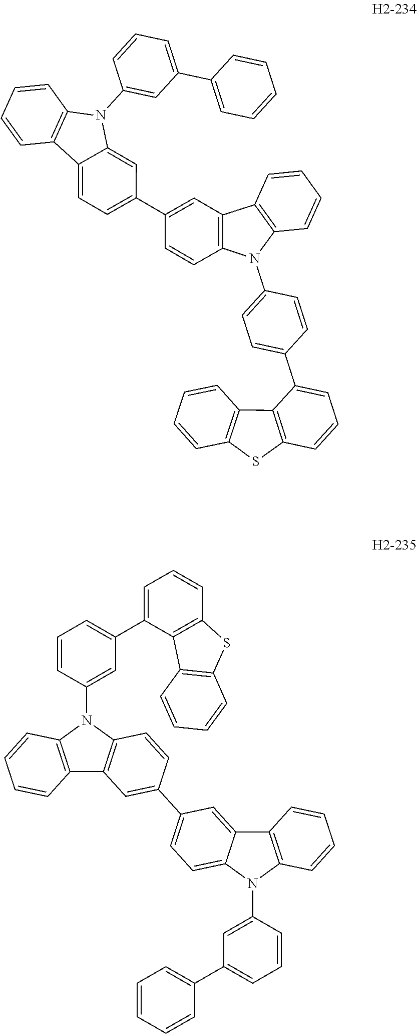 [SMILES: c1ccc(-c2cccc(-n3c4ccccc4c4ccc(-c5ccc6c(c5)c5ccccc5n6-c5ccc(-c6cccc7sc8ccccc8c67)cc5)cc43)c2)cc1 ; c1ccc(-c2cccc(-n3c4ccccc4c4cc(-c5ccc6c(c5)c5ccccc5n6-c5cccc(-c6cccc7sc8ccccc8c67)c5)ccc43)c2)cc1]

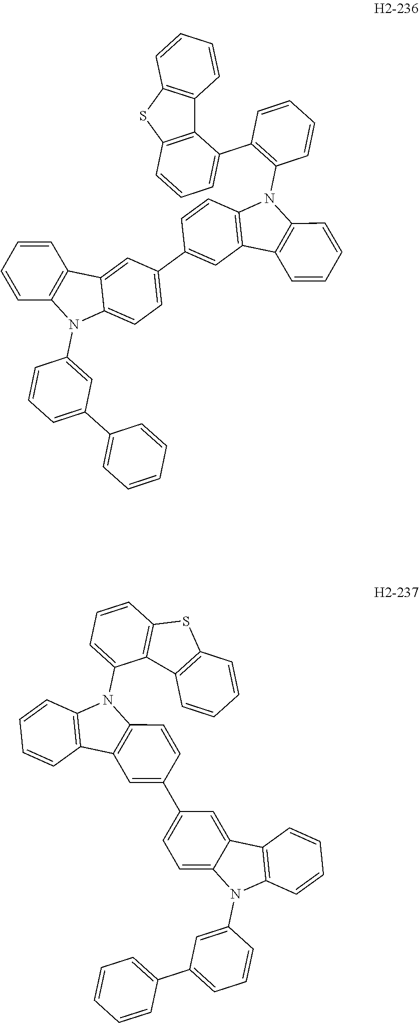 [SMILES: c1ccc(-c2cccc(-n3c4ccccc4c4cc(-c5ccc6c(c5)c5ccccc5n6-c5ccccc5-c5cccc6sc7ccccc7c56)ccc43)c2)cc1 ; c1ccc(-c2cccc(-n3c4ccccc4c4cc(-c5ccc6c(c5)c5ccccc5n6-c5cccc6sc7ccccc7c56)ccc43)c2)cc1]

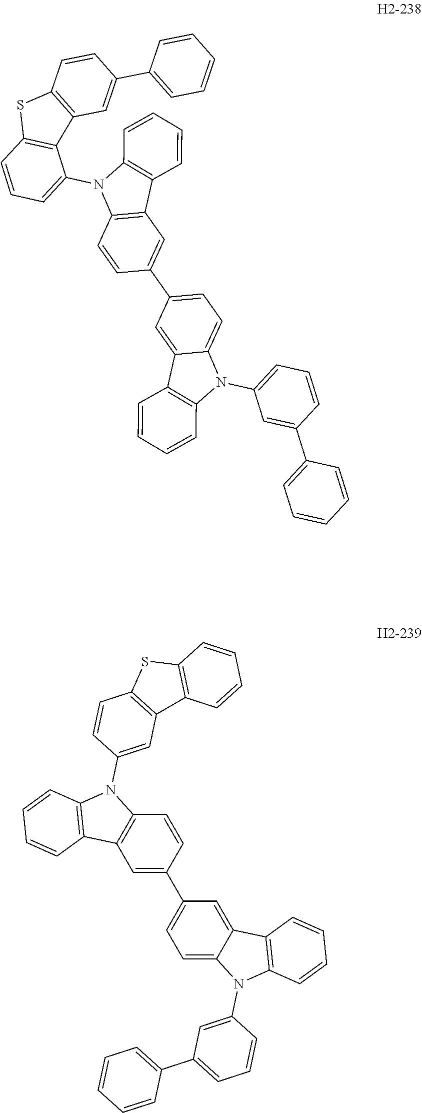 [SMILES: c1ccc(-c2cccc(-n3c4ccccc4c4cc(-c5ccc6c(c5)c5ccccc5n6-c5cccc6sc7ccc(-c8ccccc8)cc7c56)ccc43)c2)cc1 ; c1ccc(-c2cccc(-n3c4ccccc4c4cc(-c5ccc6c(c5)c5ccccc5n6-c5ccc6sc7ccccc7c6c5)ccc43)c2)cc1]

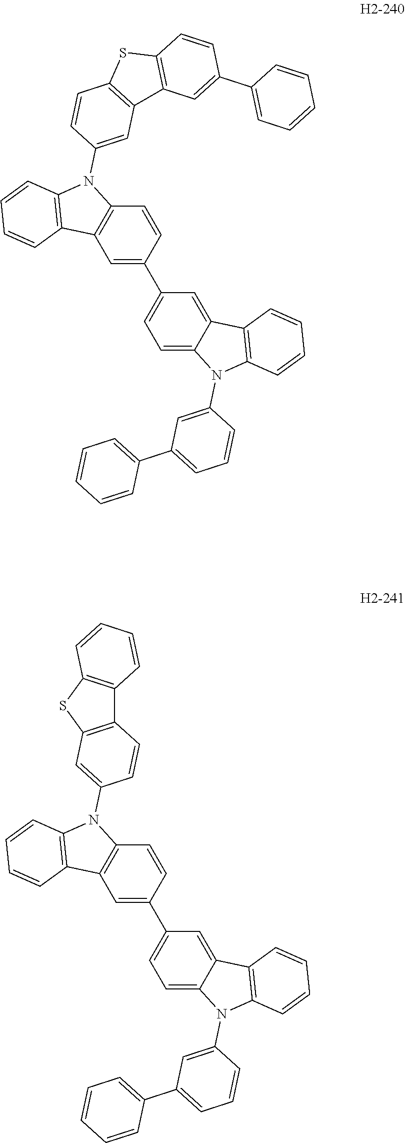 [SMILES: c1ccc(-c2cccc(-n3c4ccccc4c4cc(-c5ccc6c(c5)c5ccccc5n6-c5ccc6sc7ccc(-c8ccccc8)cc7c6c5)ccc43)c2)cc1 ; c1ccc(-c2cccc(-n3c4ccccc4c4cc(-c5ccc6c(c5)c5ccccc5n6-c5ccc6c(c5)sc5ccccc56)ccc43)c2)cc1]

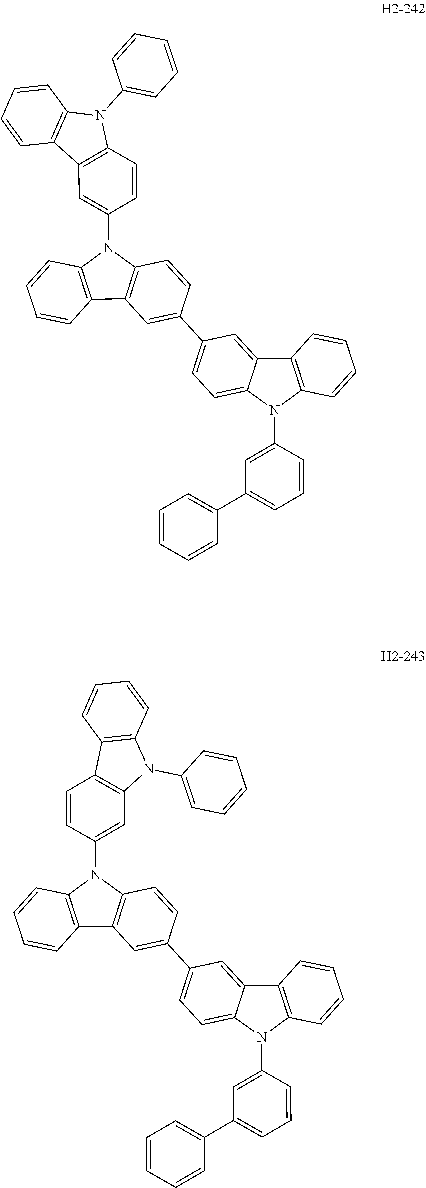 [SMILES: c1ccc(-c2cccc(-n3c4ccccc4c4cc(-c5ccc6c(c5)c5ccccc5n6-c5ccc6c(c5)c5ccccc5n6-c5ccccc5)ccc43)c2)cc1 ; c1ccc(-c2cccc(-n3c4ccccc4c4cc(-c5ccc6c(c5)c5ccccc5n6-c5ccc6c7ccccc7n(-c7ccccc7)c6c5)ccc43)c2)cc1]

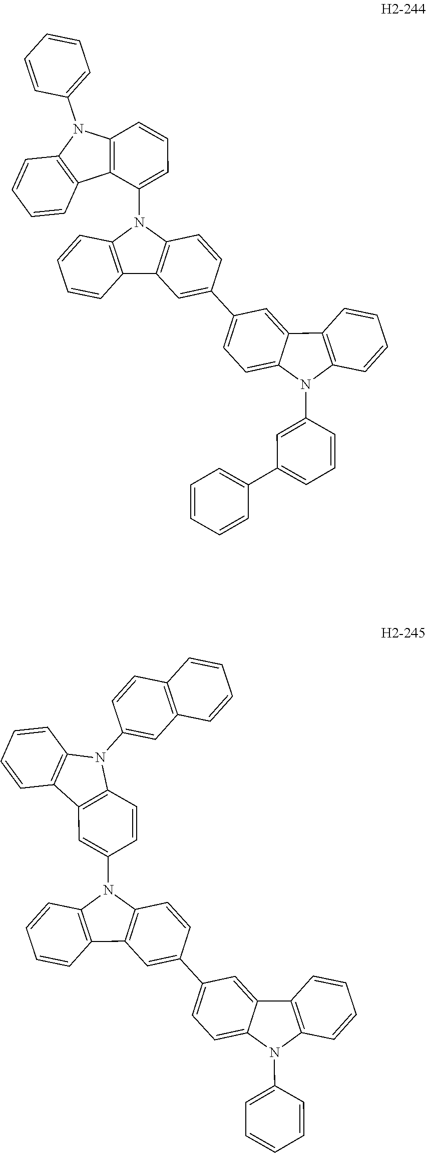 [SMILES: c1ccc(-c2cccc(-n3c4ccccc4c4cc(-c5ccc6c(c5)c5ccccc5n6-c5cccc6c5c5ccccc5n6-c5ccccc5)ccc43)c2)cc1 ; c1ccc(-n2c3ccccc3c3cc(-c4ccc5c(c4)c4ccccc4n5-c4ccc5c(c4)c4ccccc4n5-c4ccc5ccccc5c4)ccc32)cc1]

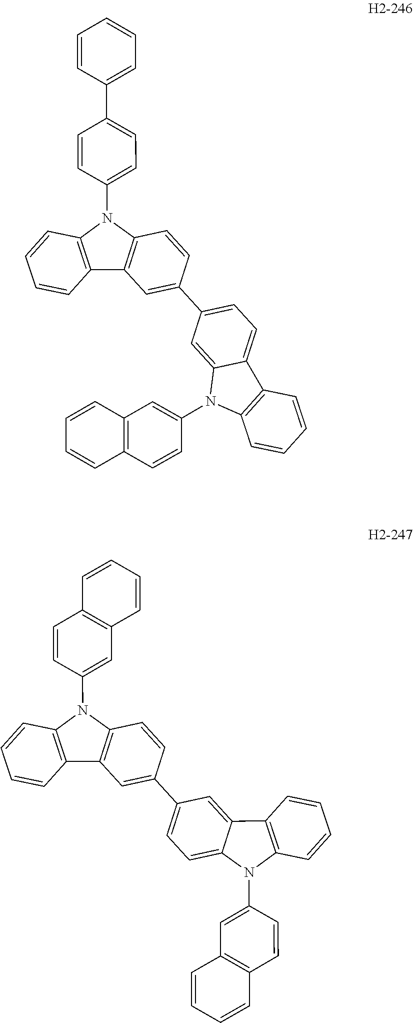 [SMILES: c1ccc(-c2ccc(-n3c4ccccc4c4cc(-c5ccc6c7ccccc7n(-c7ccc8ccccc8c7)c6c5)ccc43)cc2)cc1 ; c1ccc2cc(-n3c4ccccc4c4cc(-c5ccc6c(c5)c5ccccc5n6-c5ccc6ccccc6c5)ccc43)ccc2c1]

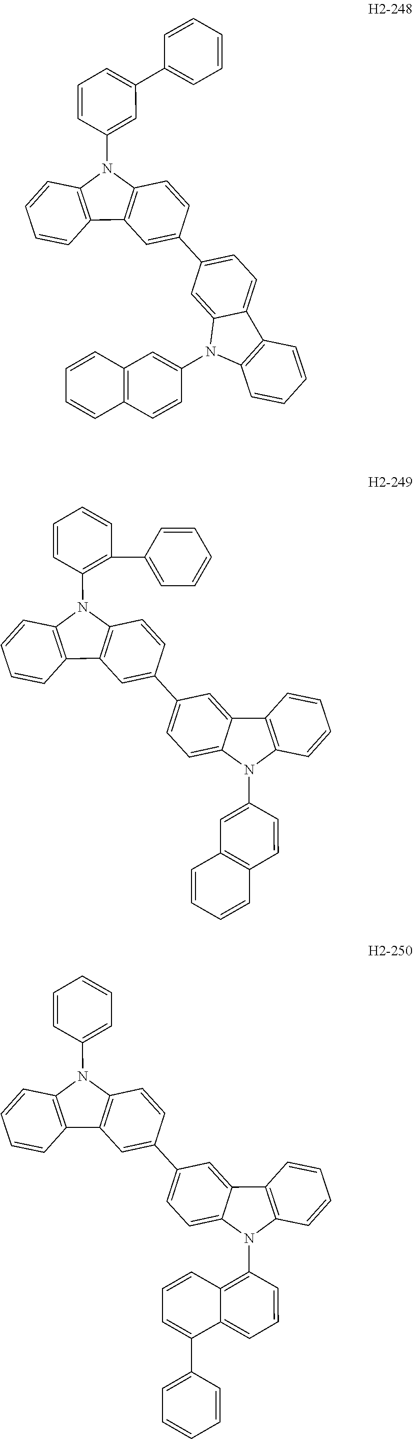 [SMILES: c1ccc(-c2cccc(-n3c4ccccc4c4cc(-c5ccc6c7ccccc7n(-c7ccc8ccccc8c7)c6c5)ccc43)c2)cc1 ; c1ccc(-c2ccccc2-n2c3ccccc3c3cc(-c4ccc5c(c4)c4ccccc4n5-c4ccc5ccccc5c4)ccc32)cc1 ; c1ccc(-c2cccc3c(-n4c5ccccc5c5cc(-c6ccc7c(c6)c6ccccc6n7-c6ccccc6)ccc54)cccc23)cc1]

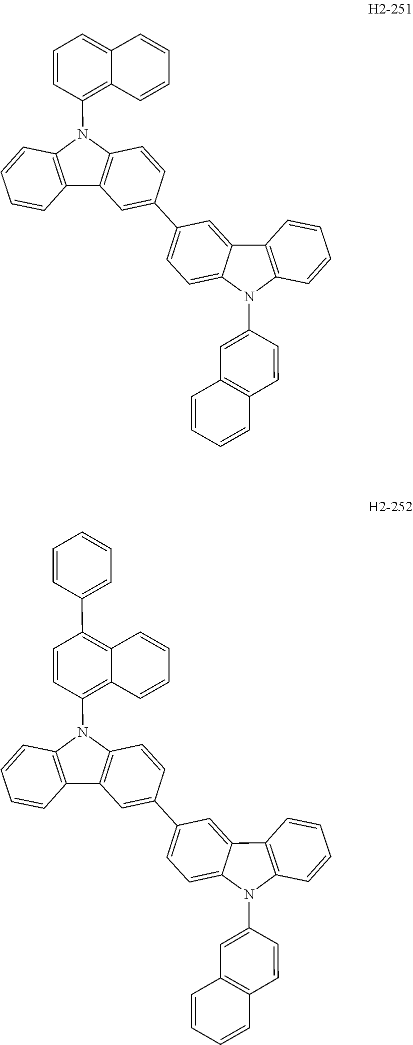 [SMILES: c1ccc2cc(-n3c4ccccc4c4cc(-c5ccc6c(c5)c5ccccc5n6-c5cccc6ccccc56)ccc43)ccc2c1 ; c1ccc(-c2ccc(-n3c4ccccc4c4cc(-c5ccc6c(c5)c5ccccc5n6-c5ccc6ccccc6c5)ccc43)c3ccccc23)cc1]

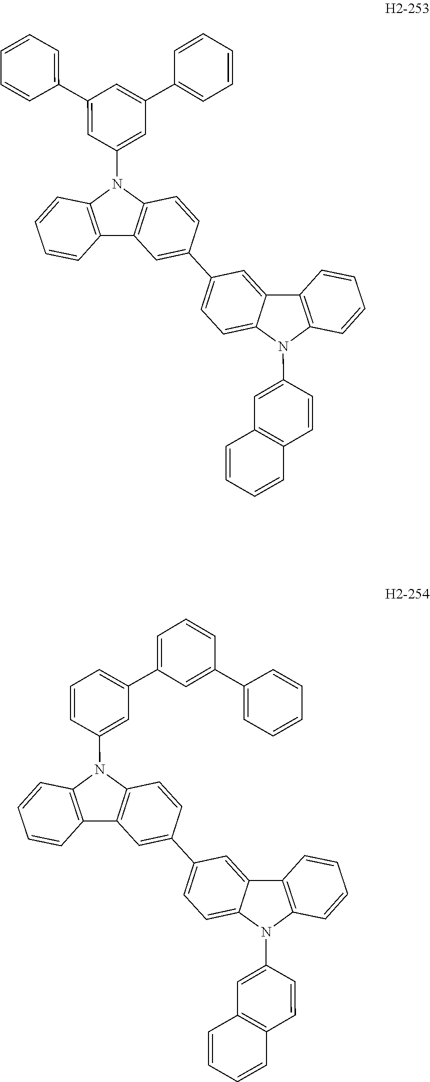 [SMILES: c1ccc(-c2cc(-c3ccccc3)cc(-n3c4ccccc4c4cc(-c5ccc6c(c5)c5ccccc5n6-c5ccc6ccccc6c5)ccc43)c2)cc1 ; c1ccc(-c2cccc(-c3cccc(-n4c5ccccc5c5cc(-c6ccc7c(c6)c6ccccc6n7-c6ccc7ccccc7c6)ccc54)c3)c2)cc1]

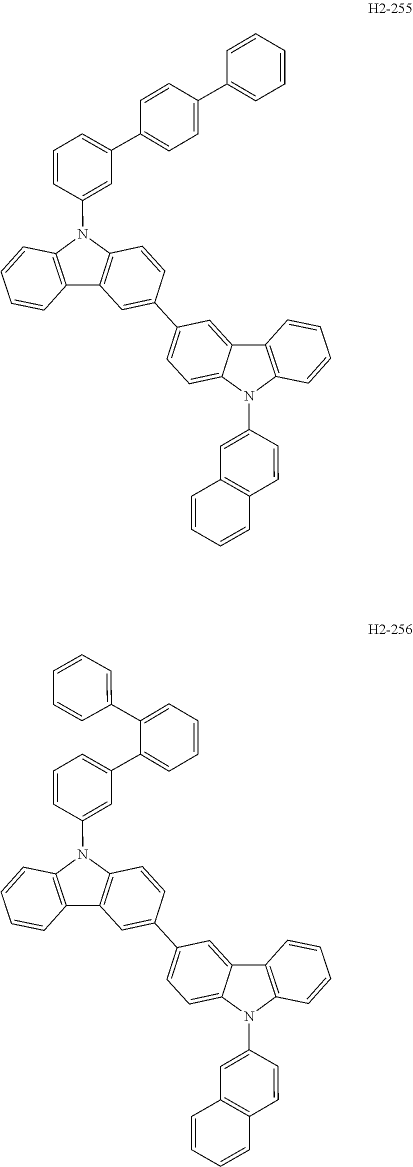 [SMILES: c1ccc(-c2ccc(-c3cccc(-n4c5ccccc5c5cc(-c6ccc7c(c6)c6ccccc6n7-c6ccc7ccccc7c6)ccc54)c3)cc2)cc1 ; c1ccc(-c2ccccc2-c2cccc(-n3c4ccccc4c4cc(-c5ccc6c(c5)c5ccccc5n6-c5ccc6ccccc6c5)ccc43)c2)cc1]

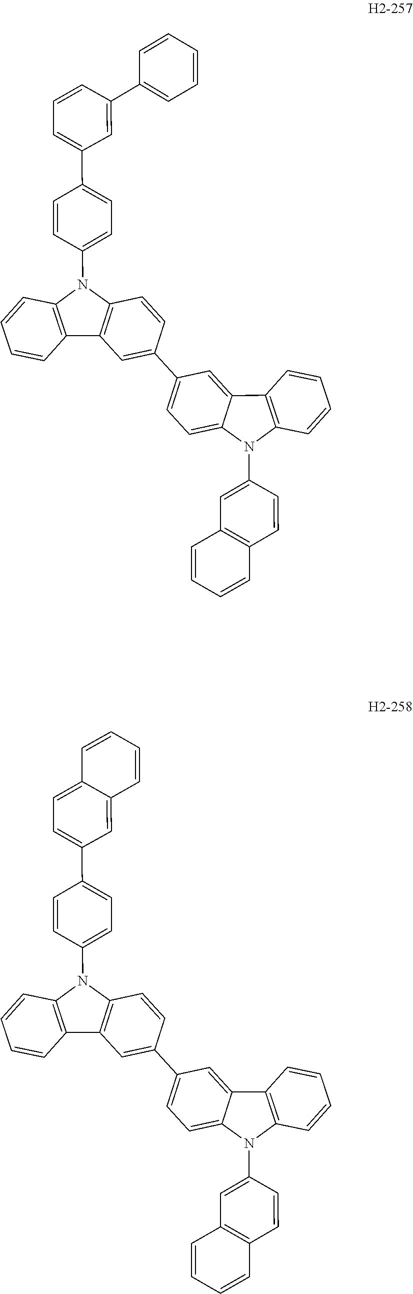 [SMILES: c1ccc(-c2cccc(-c3ccc(-n4c5ccccc5c5cc(-c6ccc7c(c6)c6ccccc6n7-c6ccc7ccccc7c6)ccc54)cc3)c2)cc1 ; c1ccc2cc(-c3ccc(-n4c5ccccc5c5cc(-c6ccc7c(c6)c6ccccc6n7-c6ccc7ccccc7c6)ccc54)cc3)ccc2c1]

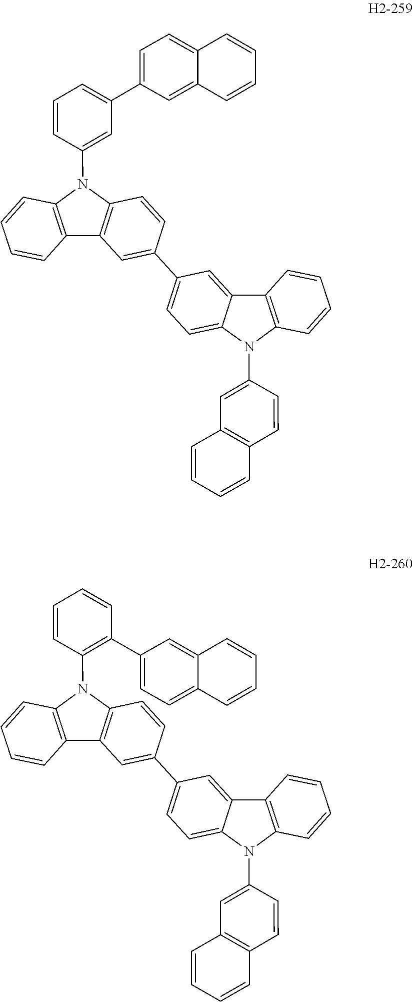 [SMILES: c1cc(-c2ccc3ccccc3c2)cc(-n2c3ccccc3c3cc(-c4ccc5c(c4)c4ccccc4n5-c4ccc5ccccc5c4)ccc32)c1 ; c1ccc(-n2c3ccccc3c3cc(-c4ccc5c(c4)c4ccccc4n5-c4ccc5ccccc5c4)ccc32)c(-c2ccc3ccccc3c2)c1]

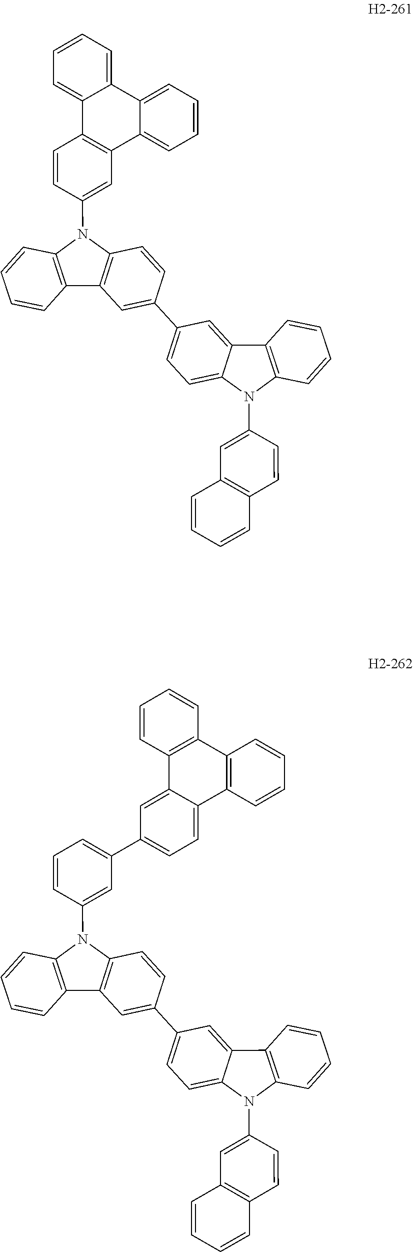 [SMILES: c1ccc2cc(-n3c4ccccc4c4cc(-c5ccc6c(c5)c5ccccc5n6-c5ccc6c7ccccc7c7ccccc7c6c5)ccc43)ccc2c1 ; c1cc(-c2ccc3c4ccccc4c4ccccc4c3c2)cc(-n2c3ccccc3c3cc(-c4ccc5c(c4)c4ccccc4n5-c4ccc5ccccc5c4)ccc32)c1]

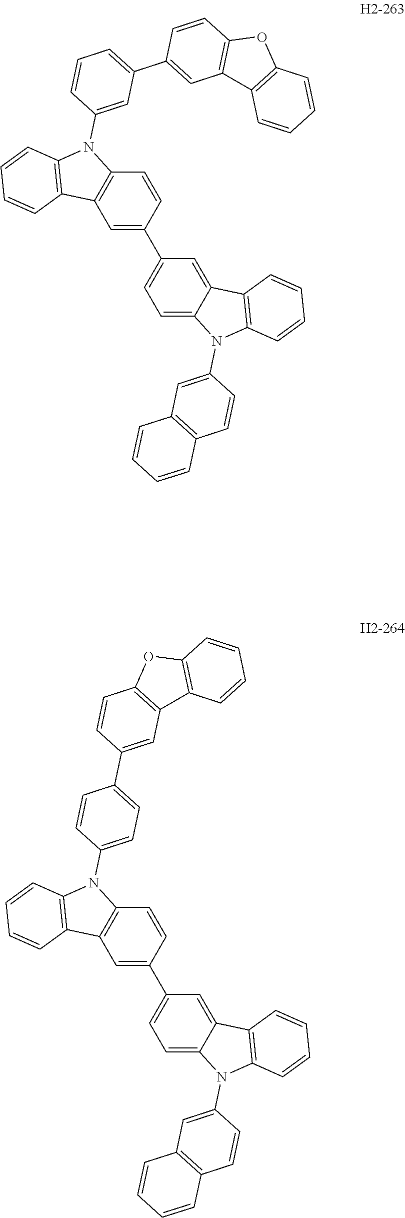 [SMILES: c1cc(-c2ccc3oc4ccccc4c3c2)cc(-n2c3ccccc3c3cc(-c4ccc5c(c4)c4ccccc4n5-c4ccc5ccccc5c4)ccc32)c1 ; c1ccc2cc(-n3c4ccccc4c4cc(-c5ccc6c(c5)c5ccccc5n6-c5ccc(-c6ccc7oc8ccccc8c7c6)cc5)ccc43)ccc2c1]

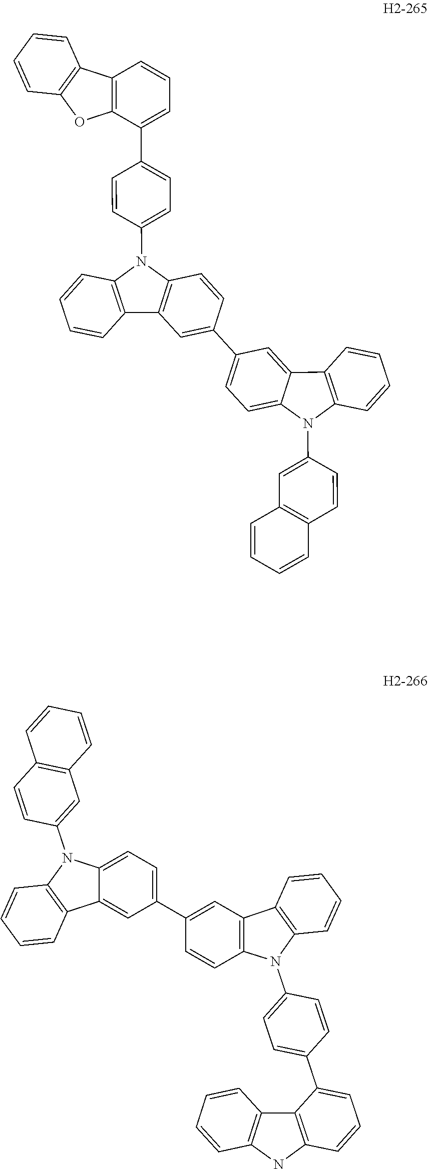 [SMILES: c1ccc2cc(-n3c4ccccc4c4cc(-c5ccc6c(c5)c5ccccc5n6-c5ccc(-c6cccc7c6oc6ccccc67)cc5)ccc43)ccc2c1 ; c1ccc2cc(-n3c4ccccc4c4cc(-c5ccc6c(c5)c5ccccc5n6-c5ccc(-c6cccc7[nH]c8ccccc8c67)cc5)ccc43)ccc2c1]

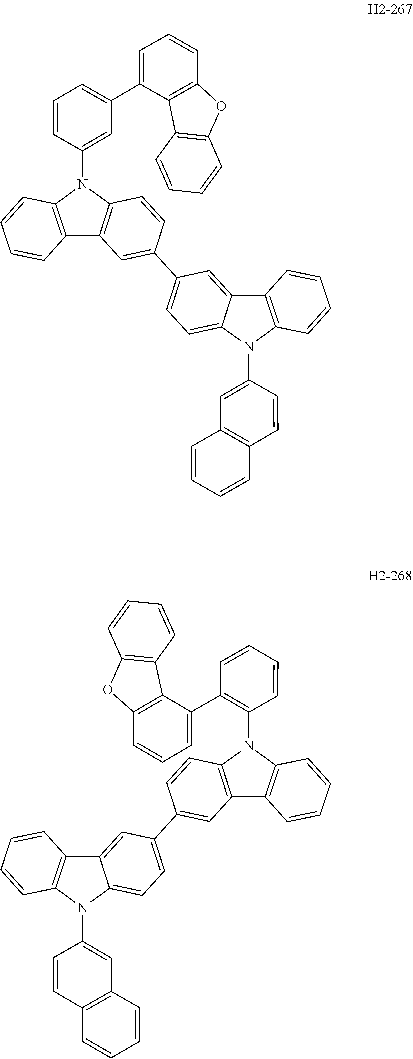 [SMILES: c1cc(-c2cccc3oc4ccccc4c23)cc(-n2c3ccccc3c3cc(-c4ccc5c(c4)c4ccccc4n5-c4ccc5ccccc5c4)ccc32)c1 ; c1ccc(-n2c3ccccc3c3cc(-c4ccc5c(c4)c4ccccc4n5-c4ccc5ccccc5c4)ccc32)c(-c2cccc3oc4ccccc4c23)c1]

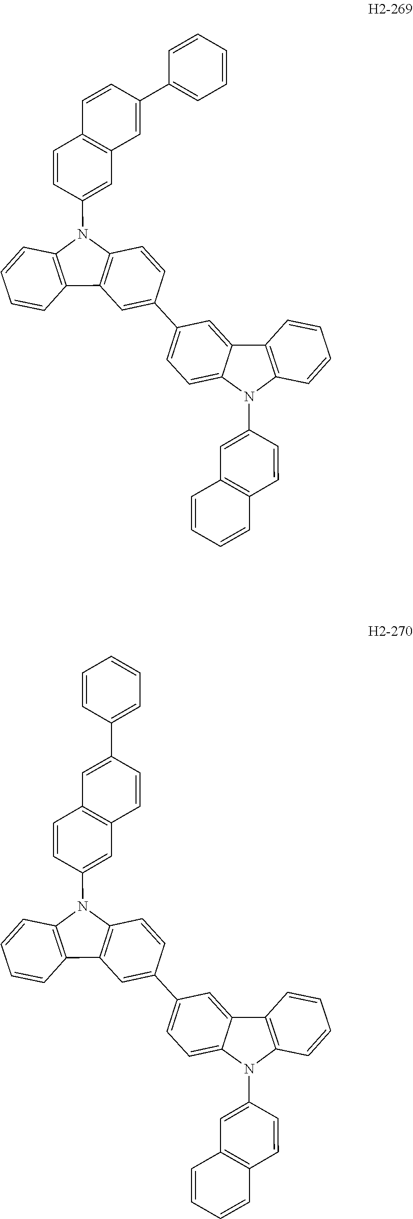 [SMILES: c1ccc(-c2ccc3ccc(-n4c5ccccc5c5cc(-c6ccc7c(c6)c6ccccc6n7-c6ccc7ccccc7c6)ccc54)cc3c2)cc1 ; c1ccc(-c2ccc3cc(-n4c5ccccc5c5cc(-c6ccc7c(c6)c6ccccc6n7-c6ccc7ccccc7c6)ccc54)ccc3c2)cc1]

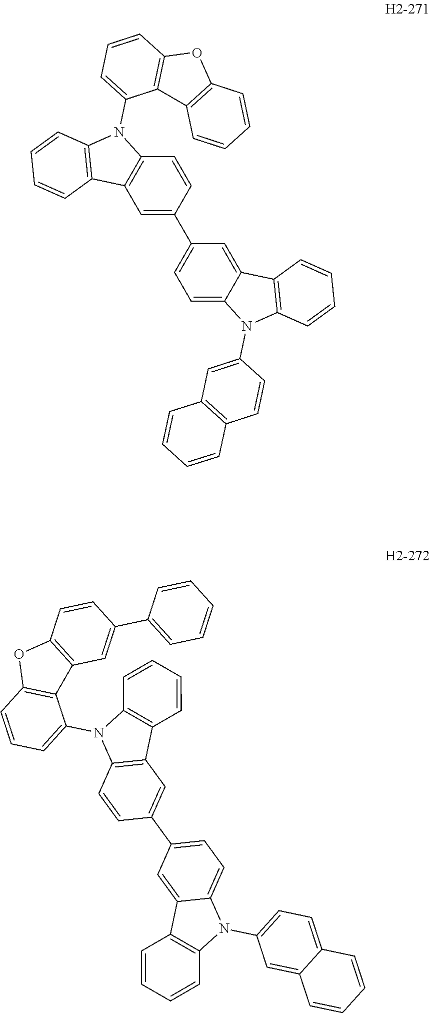 [SMILES: c1ccc2cc(-n3c4ccccc4c4cc(-c5ccc6c(c5)c5ccccc5n6-c5cccc6oc7ccccc7c56)ccc43)ccc2c1 ; c1ccc(-c2ccc3oc4cccc(-n5c6ccccc6c6cc(-c7ccc8c(c7)c7ccccc7n8-c7ccc8ccccc8c7)ccc65)c4c3c2)cc1]

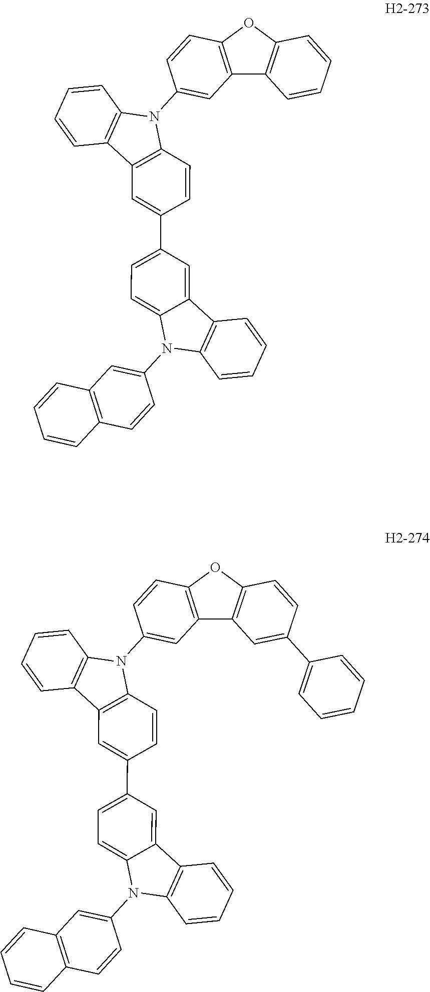 [SMILES: c1ccc2cc(-n3c4ccccc4c4cc(-c5ccc6c(c5)c5ccccc5n6-c5ccc6oc7ccccc7c6c5)ccc43)ccc2c1 ; c1ccc(-c2ccc3oc4ccc(-n5c6ccccc6c6cc(-c7ccc8c(c7)c7ccccc7n8-c7ccc8ccccc8c7)ccc65)cc4c3c2)cc1]

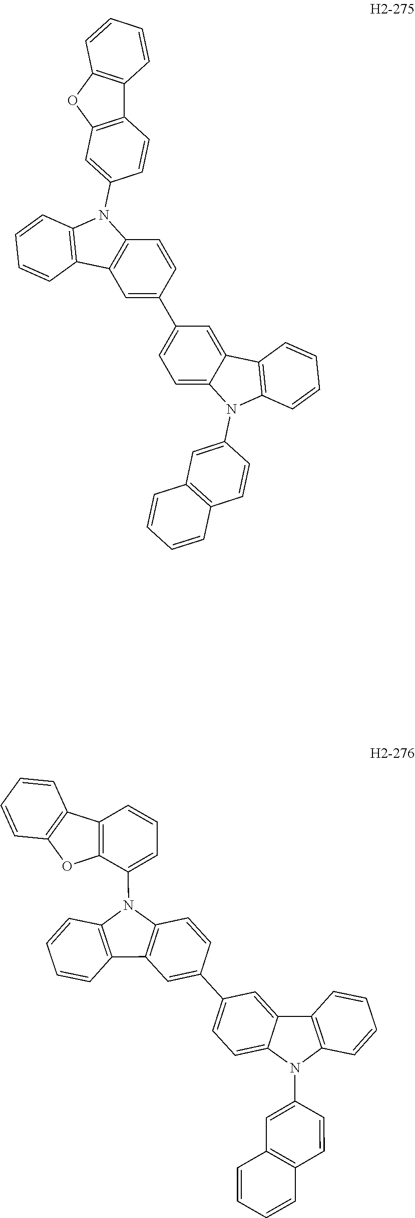 [SMILES: c1ccc2cc(-n3c4ccccc4c4cc(-c5ccc6c(c5)c5ccccc5n6-c5ccc6c(c5)oc5ccccc56)ccc43)ccc2c1 ; c1ccc2cc(-n3c4ccccc4c4cc(-c5ccc6c(c5)c5ccccc5n6-c5cccc6c5oc5ccccc56)ccc43)ccc2c1]

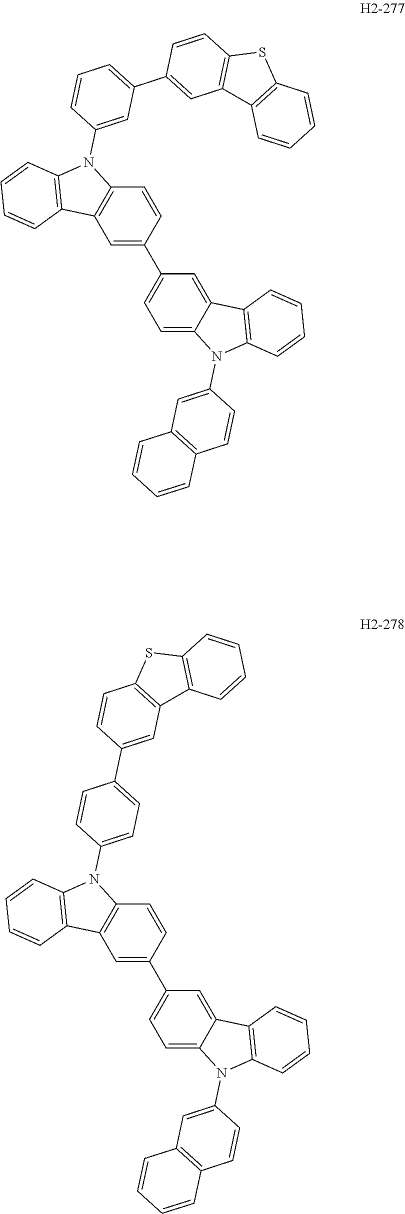 [SMILES: c1cc(-c2ccc3sc4ccccc4c3c2)cc(-n2c3ccccc3c3cc(-c4ccc5c(c4)c4ccccc4n5-c4ccc5ccccc5c4)ccc32)c1 ; c1ccc2cc(-n3c4ccccc4c4cc(-c5ccc6c(c5)c5ccccc5n6-c5ccc(-c6ccc7sc8ccccc8c7c6)cc5)ccc43)ccc2c1]

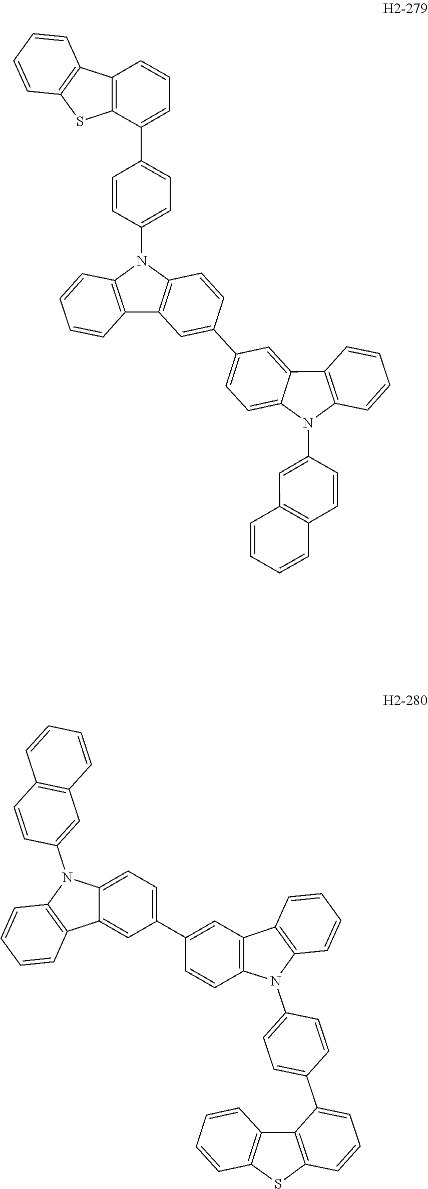 [SMILES: c1ccc2cc(-n3c4ccccc4c4cc(-c5ccc6c(c5)c5ccccc5n6-c5ccc(-c6cccc7c6sc6ccccc67)cc5)ccc43)ccc2c1 ; c1ccc2cc(-n3c4ccccc4c4cc(-c5ccc6c(c5)c5ccccc5n6-c5ccc(-c6cccc7sc8ccccc8c67)cc5)ccc43)ccc2c1]

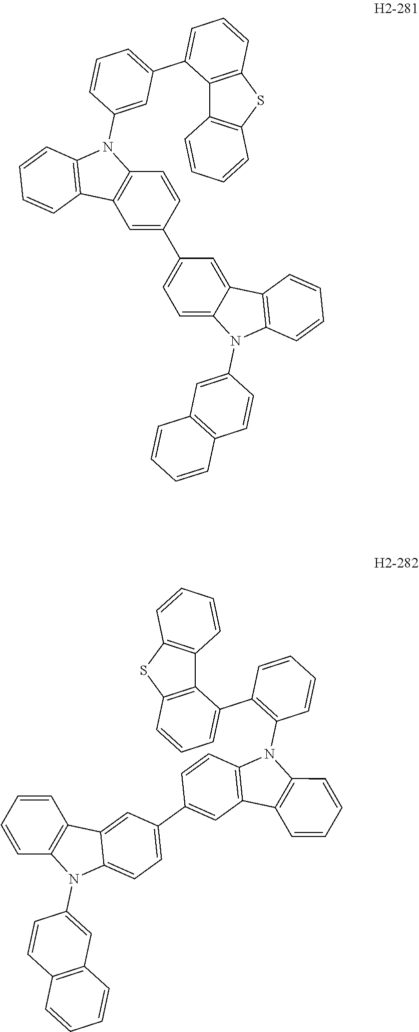 [SMILES: c1cc(-c2cccc3sc4ccccc4c23)cc(-n2c3ccccc3c3cc(-c4ccc5c(c4)c4ccccc4n5-c4ccc5ccccc5c4)ccc32)c1 ; c1ccc(-n2c3ccccc3c3cc(-c4ccc5c(c4)c4ccccc4n5-c4ccc5ccccc5c4)ccc32)c(-c2cccc3sc4ccccc4c23)c1]

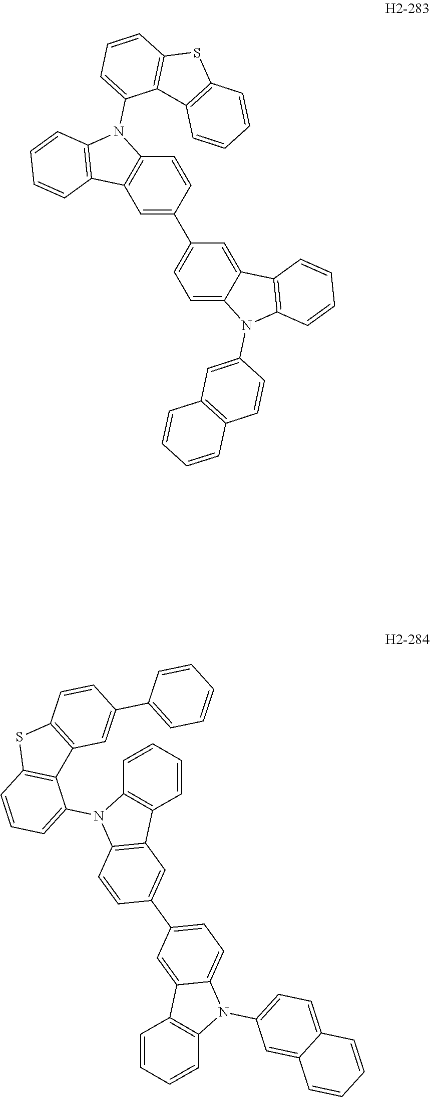 [SMILES: c1ccc2cc(-n3c4ccccc4c4cc(-c5ccc6c(c5)c5ccccc5n6-c5cccc6sc7ccccc7c56)ccc43)ccc2c1 ; c1ccc(-c2ccc3sc4cccc(-n5c6ccccc6c6cc(-c7ccc8c(c7)c7ccccc7n8-c7ccc8ccccc8c7)ccc65)c4c3c2)cc1]

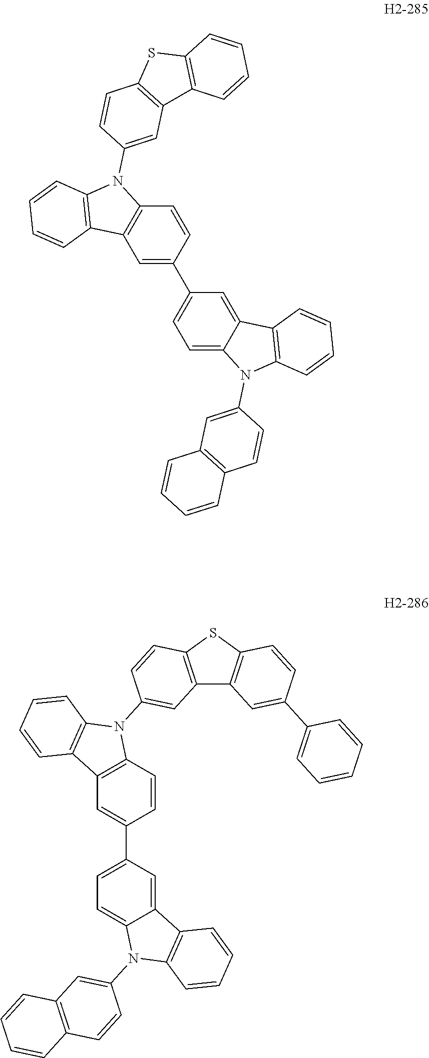 [SMILES: c1ccc2cc(-n3c4ccccc4c4cc(-c5ccc6c(c5)c5ccccc5n6-c5ccc6sc7ccccc7c6c5)ccc43)ccc2c1 ; c1ccc(-c2ccc3sc4ccc(-n5c6ccccc6c6cc(-c7ccc8c(c7)c7ccccc7n8-c7ccc8ccccc8c7)ccc65)cc4c3c2)cc1]

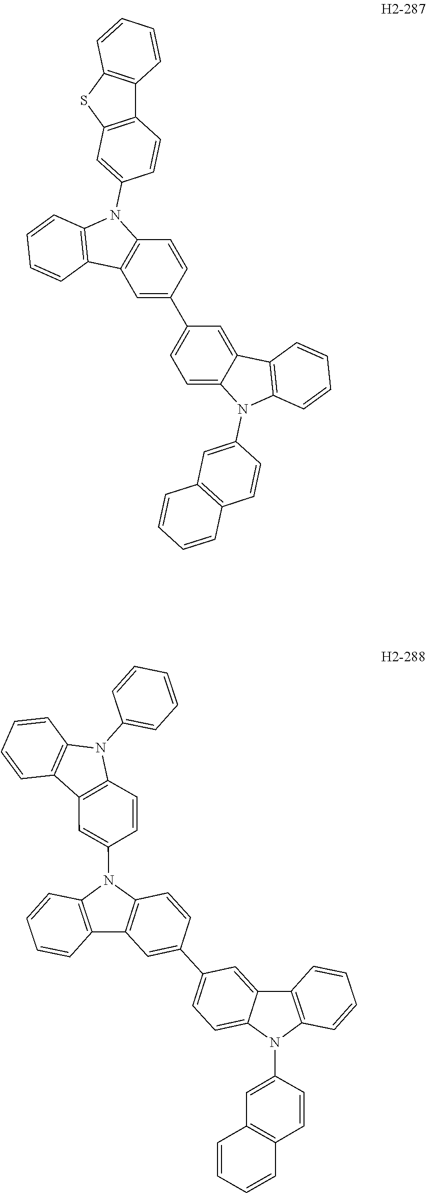 [SMILES: c1ccc2cc(-n3c4ccccc4c4cc(-c5ccc6c(c5)c5ccccc5n6-c5ccc6c(c5)sc5ccccc56)ccc43)ccc2c1 ; c1ccc(-n2c3ccccc3c3cc(-n4c5ccccc5c5cc(-c6ccc7c(c6)c6ccccc6n7-c6ccc7ccccc7c6)ccc54)ccc32)cc1]

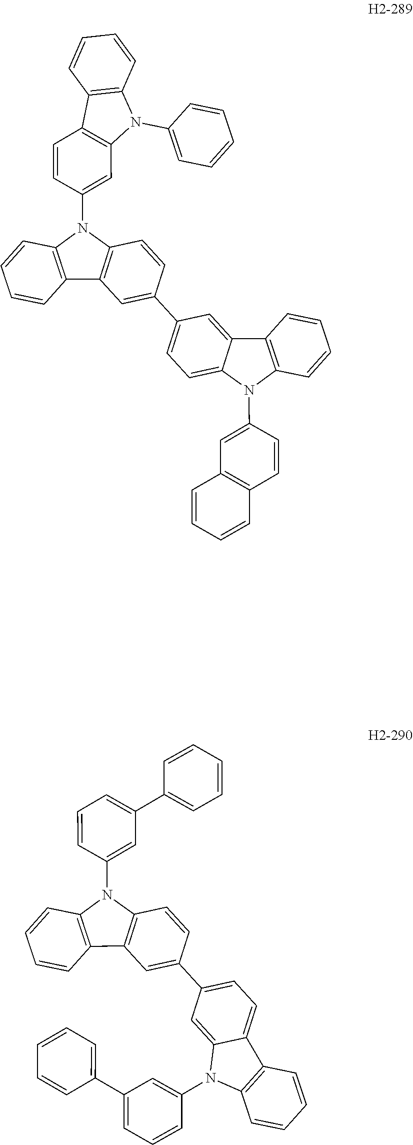 [SMILES: c1ccc(-n2c3ccccc3c3ccc(-n4c5ccccc5c5cc(-c6ccc7c(c6)c6ccccc6n7-c6ccc7ccccc7c6)ccc54)cc32)cc1 ; c1ccc(-c2cccc(-n3c4ccccc4c4cc(-c5ccc6c7ccccc7n(-c7cccc(-c8ccccc8)c7)c6c5)ccc43)c2)cc1]

In the above compounds, Dn means that n hydrogens have been substituted by deuterium.

According to another embodiment of the present disclosure, the present disclosure provides an organic electroluminescent compound represented by Formula 11. A more specific description of the compound represented by Formula 11 of the present disclosure is as follows.

In Formula 11 above, each of X′₁ to X′₃ each independently represent N or CR′ₐ; provided that at least two of X′₁ to X′₃ are N. According to one embodiment of the present disclosure, any two of X′₁ to X′₃ are N and the other is CR′ₐ. According to another embodiment of the present disclosure, all of X′₁ to X′₃ are N. R′ₐ is hydrogen or deuterium.

In Formula 11 above, Ar′₁ to Ar′₃ each independently represent (C6-C30)aryl unsubstituted or substituted with at least one of deuterium and (C6-C30)aryl, or are represented by Formula A above; provided that at least one of Ar′₁ to Ar′₃ is(are) represented by Formula A above. According to one embodiment of the present disclosure, Ar′₁ to Ar′₃ each independently represent (C6-C18)aryl unsubstituted or substituted with deuterium, or are represented by Formula A above. According to another embodiment of the present disclosure, any one of Ar′₁ to Ar′₃ is represented by Formula A above. For example, Ar′₁ to Ar′₃ may be each independently represented by Formula A above, or may be phenyl, biphenyl, or terphenyl, etc. They may be further substituted with deuterium.

In Formula A above, L′₁ is a single bond, or a substituted or unsubstituted (C6-C12)arylene. For example, L′₁ may be a single bond.

In Formula A above, Cz is a substituted or unsubstituted carbazolylene. According to one embodiment of the present disclosure, Cz is a carbazolylene unsubstituted or substituted with at least one of deuterium and (C6-C30)aryl. For example, Cz is a carbazolylene unsubstituted or substituted with at least one of deuterium and phenyl.

In Formula A above, L′₂ is a substituted or unsubstituted (C6-C12)arylene. According to one embodiment of the present disclosure, L′₂ is (C6-C12)arylene unsubstituted or substituted with deuterium. For example, L′₂ may be phenylene unsubstituted or substituted with deuterium.

In Formula A above, HAr is a substituted or unsubstituted dibenzofuranyl, a substituted or unsubstituted carbazolyl, or a substituted or unsubstituted dibenzothiophenyl. For example, HAr may be dibenzofuranyl or dibenzothiophenyl, which may be further substituted with deuterium.

According to one embodiment of the present disclosure, Formula A of the present disclosure may be represented by the following Formula A-1.

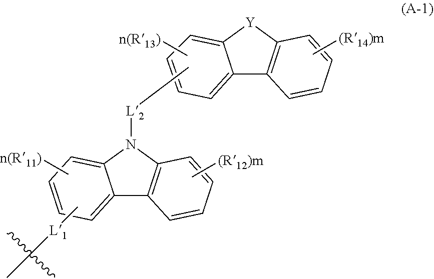

In Formula A-1 above, the definitions and preferred embodiments of L′₁ and L′₂ are the same as in Formula A above.

In Formula A-1 above, Y represents —O—, —S—, or —N(R′₁)—. For example, Y is —O— or —S—.

In Formula A-1 above, R′₁ represents a substituted or unsubstituted (C6-C12)aryl, or a substituted or unsubstituted dibenzofuranyl, a substituted or unsubstituted carbazolyl, or a substituted or unsubstituted dibenzothiophenyl.

In Formula A-1 above, R′₁₁ to R′₁₄ each independently represent hydrogen, deuterium, a substituted or unsubstituted (C6-C12)aryl, a substituted or unsubstituted dibenzofuranyl, a substituted or unsubstituted carbazolyl, or a substituted or unsubstituted dibenzothiophenyl. According to one embodiment of the present disclosure, R′₁₁ to R′₁₄ each independently represent hydrogen, deuterium, or (C6-C12)aryl unsubstituted or substituted with deuterium. For example, R′₁₁ to R′₁₄ may be each independently hydrogen, deuterium, or phenyl unsubstituted or substituted with deuterium.

In Formula A-1 above, n is each independently an integer from 1 to 3, m is each independently an integer from 1 to 4, and each of R′₁₁ to R′₁₄ may be the same or different from each other.

According to one embodiment of the present disclosure, the compound represented by Formula 11 may be selected from the group consisting of compounds C-51 to C-65, C-67 to C-85, C-87 to C-150, C-196 to C-200, and C-226 to C-236, but is not limited thereto.

According to another embodiment of the present disclosure, the present disclosure provides a plurality of host materials comprising at least one first host compound and at least one second host compound, wherein the first host compound is represented by Formula 12 of the present disclosure and the second host compound is represented by Formula 13 of the present disclosure, and wherein the first host compound and the second host compound do not contain deuterium.

The compound represented by Formula 12 of the present disclosure will be described in more detail as follows.

In Formula 12 above, X₁ to X′₃ each independently represent N or CH; provided that at least two of X₁ to X′₃ represent N. According to one embodiment of the present disclosure, all of X₁ to X′₃ are N.

In Formula 12 above, Ar′₁ to Ar′₃ each independently represent (C6-C30)aryl unsubstituted or substituted with C6-C30)aryl, or are represented by Formula A above; provided that at least one of Ar′₁ to Ar′₃ is represented by Formula A above. According to one embodiment of the present disclosure, Ar′₁ and Ar′₃ are each independently, unsubstituted (C6-C18)aryl or represented by Formula A above. According to another embodiment of the present disclosure, any one of Ar′₁ to Ar′₃ is represented by Formula A above. For example, Ar′₁ to Ar′₃ may be each independently represented by Formula A above, or may be phenyl, biphenyl, or terphenyl, etc.

In Formula A above, L′₁ represents a single bond, or a substituted or unsubstituted (C6-C12)arylene. For example, L′₁ may be a single bond.

In Formula A above, Cz is a substituted or unsubstituted carbazolylene. According to one embodiment of the present disclosure, Cz is a carbazolylene unsubstituted or substituted with at least one of (C6-C30)aryl. For example, Cz is a carbazolylene unsubstituted or substituted with phenyl.

In Formula A above, L′₂ is a substituted or unsubstituted (C6-C12)arylene. According to one embodiment of the present disclosure, L′₂ is an unsubstituted (C6-C12)arylene. For example, L′₂ may be an unsubstituted phenylene.

In Formula A above, HAr is a substituted or unsubstituted dibenzofuranyl, a substituted or unsubstituted carbazolyl, or a substituted or unsubstituted dibenzothiophenyl.

For example, HAr may be an unsubstituted dibenzofuranyl or an unsubstituted dibenzothiophenyl.

According to one embodiment of the present disclosure, the compound represented by Formula 12 may be selected from the group consisting of compounds C-51 to C-65, C-67 to C-85, C-87 to C-130, C-196 to C-200, and C-227 to C-236, but not limited thereto.

The compound represented by Formula 13 of the present disclosure will be described in more detail as follows.

In Formula 13 above, A′₁ and A′₂ may be each independently a substituted or unsubstituted, (C6-C30)aryl, dibenzofuranyl, dibenzothiophenyl, or carbazolyl, and preferably a substituted or unsubstituted, (C6-C25)aryl, dibenzofuranyl, dibenzothiophenyl, or carbazolyl. Specifically, A′₁ and A′₂ may be each independently a substituted or unsubstituted, phenyl, p-biphenyl, m-biphenyl, o-biphenyl, p-terphenyl, m-terphenyl, o-terphenyl, naphthyl, fluorenyl, benzofluorenyl, triphenylenyl, fluoranthenyl, phenanthrenyl, dibenzofuranyl, dibenzothiophenyl, or carbazolyl. The substituents of said substituents may be one or more of (C6-C30)aryl and (3- to 30-membered)heteroaryl, preferably one or more of (C6-C18)aryl and (5- to 20-membered)heteroaryl. For example, A′₁ and A′₂ may be each independently phenyl unsubstituted or substituted with at least one of naphthyl, triphenylenyl, dibenzofuranyl, and dibenzothiophenyl; naphthyl unsubstituted or substituted with phenyl; p-biphenyl; m-biphenyl; o-biphenyl; o-terphenyl; m-terphenyl; p-terphenyl; triphenylenyl; dibenzofuranyl unsubstituted or substituted with phenyl; dibenzothiophenyl unsubstituted or substituted with phenyl; or carbazolyl unsubstituted or substituted with one or more of phenyl, and naphthyl.

In Formula 13 above, any one of X′₁₅ to X′₁₈ and any one of X′₁₉ to X′₂₂ are linked together to form a single bond. X′₁₁ to X′₁₄, X′₂₃ to X′₂₆, and X′₁₅ to X′₂₂, which do not form a single bond, may be each independently hydrogen, a substituted or unsubstituted (C6-C30)aryl, or a substituted or unsubstituted (3- to 30-membered)heteroaryl; or may be linked to an adjacent substituent to form a ring. According to one embodiment of the present disclosure, X′₁ to X′₁₄, X′₂₃ to X′₂₆, and X′₁₅ to X′₂₂, which do not form a single bond, may be each independently hydrogen, a substituted or unsubstituted (C6-C30)aryl, or a substituted or unsubstituted (5- to 30-membered)heteroaryl. For example, X′₁ to X′₁₄, X′₂₃ to X′₂₆, and X′₁₅ to X′₂₂, which do not form a single bond, may be hydrogen.

According to one embodiment of the present disclosure, the compound represented by Formula 13 may be one or more selected from the group consisting of compounds H2-146 to H2-290, but is not limited thereto.

According to another embodiment of the present disclosure, the present disclosure provides three or more host materials comprising one or more first host compounds and one or more second host compounds. The structural backbone of the first host compounds and the second host compounds is the same, but the number of hydrogen and deuterium atoms in each compound is different

According to one embodiment of the present disclosure, the first host compound does not contain deuterium, and the second host compound may contain deuterium.

According to one embodiment, the structural backbone of the first host compound and the second host compound is represented by Formula 2 above.

According to one embodiment of the present disclosure, the plurality of host materials further comprises a third host compound, wherein the third host compound is represented by the following Formula 1-11.

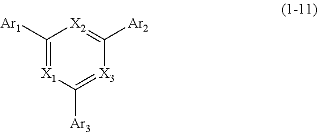

In Formula 1-11,
X₁ to X₃ each independently represent N or CRₐ; provided that at least two of X₁ to X₃ are N;
Rₐ represents hydrogen or deuterium; and
Ar₁ to Ar₃ each independently represent, a substituted or unsubstituted (C6-C30)aryl or a substituted or unsubstituted (3- to 30-membered)heteroaryl.

Hereinafter, an organic electroluminescent device to which the aforementioned plurality of host materials and/or organic electroluminescent compound is (are) applied, will be described.

The organic electroluminescent device according to one embodiment includes a first electrode; a second electrode; and at least one light-emitting layer(s) interposed between the first electrode and the second electrode, wherein at least one of the light-emitting layer(s) comprises a plurality of host materials of the present disclosure.

According to one embodiment of the present disclosure, the plurality of host materials of the present disclosure comprises at least one first host compound represented by Formula 1 and at least one second host compound represented by Formula 2, for example, at least one compound(s) of compounds C-1 to C-236, which is a first host compound, and at least one compound(s) of compounds H2-1 to H2-290, which is a second host compound. The plurality of host materials may be included in the same organic layer, for example the same light-emitting layer, or may be included in different light-emitting layers, respectively.

According to one embodiment of the present disclosure, the plurality of host materials of the present disclosure comprises at least one first host compound represented by Formula 12 and at least one second host compound represented by Formula 13, for example, at least one compound(s) of compounds C-51 to C-65, C-67 to C-85, C-87 to C-130, C-196 to C-200, and C-227 to C-236, which is a first host compound, and at least one compound(s) of compounds H2-146 to H2-290, which is a second host compound. The plurality of host materials may be included in the same organic layer, for example the same light-emitting layer, or may be included in different light-emitting layers, respectively.

According to another embodiment, the present disclosure may comprise an organic electroluminescent compound represented by Formula 11 in the light-emitting layer as a host material, an electron transport layer material, or an electron buffer layer material.

The organic layer may further comprise at least one layer selected from a hole injection layer, a hole transport layer, a hole auxiliary layer, a light-emitting auxiliary layer, an electron transport layer, an electron injection layer, an interlayer, a hole blocking layer, an electron blocking layer and an electron buffer layer, in addition to the light-emitting layer. The organic layer may further comprise an amine-based compound and/or an azine-based compound other than the light-emitting material according to the present disclosure. Specifically, the hole injection layer, the hole transport layer, the hole auxiliary layer, the light-emitting layer, the light-emitting auxiliary layer, or the electron blocking layer may contain the amine-based compound, e.g., an arylamine-based compound and a styrylarylamine-based compound, etc., as a hole injection material, a hole transport material, a hole auxiliary material, a light-emitting material, a light-emitting auxiliary material, or an electron blocking material. Also, the electron transport layer, the electron injection layer, the electron buffer layer, or the hole blocking layer may contain the azine-based compound as an electron transport material, an electron injection material, an electron buffer material, or a hole blocking material. Also, the organic layer may further comprise at least one metal selected from the group consisting of metals of Group 1, metals of Group 2, transition metals of the 4^{th} period, transition metals of the 5^{th} period, lanthanides, and organic metals of the d-transition elements of the Periodic Table, or at least one complex compound comprising such a metal.

The plurality of host materials according to one embodiment may be used as light-emitting materials for a white organic light-emitting device. The white organic light-emitting device has suggested various structures such as a parallel side-by-side arrangement method, a stacking arrangement method, or CCM (color conversion material) method, etc., according to the arrangement of R (Red), G (Green), YG (yellowish green), or B (blue) light-emitting units. In addition, the plurality of host materials according to one embodiment may also be applied to the organic electroluminescent device comprising a QD (quantum dot).

One of the first electrode and the second electrode may be an anode and the other may be a cathode. Wherein, the first electrode and the second electrode may each be formed as a transmissive conductive material, a transflective conductive material, or a reflective conductive material. The organic electroluminescent device may be a top emission type, a bottom emission type, or a both-sides emission type according to the kinds of the material forming the first electrode and the second electrode.

A hole injection layer, a hole transport layer, an electron blocking layer, or a combination thereof can be used between the anode and the light-emitting layer. The hole injection layer may be multi-layers in order to lower the hole injection barrier (or hole injection voltage) from the anode to the hole transport layer or the electron blocking layer, wherein each of the multi-layers may use two compounds simultaneously. Also, the hole injection layer may be doped as a p-dopant. Also, the electron blocking layer may be placed between the hole transport layer (or hole injection layer) and the light-emitting layer, and can confine the excitons within the light-emitting layer by blocking the overflow of electrons from the light-emitting layer to prevent a light-emitting leakage. The hole transport layer or the electron blocking layer may be multi-layers, and wherein each layer may use a plurality of compounds.

An electron buffer layer, a hole blocking layer, an electron transport layer, an electron injection layer, or a combination thereof can be used between the light-emitting layer and the cathode. The electron buffer layer may be multi-layers in order to control the injection of the electron and improve the interfacial properties between the light-emitting layer and the electron injection layer, wherein each of the multi-layers may use two compounds simultaneously. The hole blocking layer may be placed between the electron transport layer (or electron injection layer) and the light-emitting layer, and blocks the arrival of holes to the cathode, thereby improving the probability of recombination of electrons and holes in the light-emitting layer. The hole blocking layer or the electron transport layer may also be multi-layers, wherein each layer may use a plurality of compounds. Also, the electron injection layer may be doped as an n-dopant.

The light-emitting auxiliary layer may be placed between the anode and the light-emitting layer, or between the cathode and the light-emitting layer. When the light-emitting auxiliary layer is placed between the anode and the light-emitting layer, it can be used for promoting the hole injection and/or the hole transport, or for preventing the overflow of electrons. When the light-emitting auxiliary layer is placed between the cathode and the light-emitting layer, it can be used for promoting the electron injection and/or the electron transport, or for preventing the overflow of holes. In addition, the hole auxiliary layer may be placed between the hole transport layer (or hole injection layer) and the light-emitting layer, and may be effective to promote or block the hole transport rate (or the hole injection rate), thereby enabling the charge balance to be controlled. When an organic electroluminescent device includes two or more hole transport layers, the hole transport layer, which is further included, may be used as the hole auxiliary layer or the electron blocking layer. The light-emitting auxiliary layer, the hole auxiliary layer, or the electron blocking layer may have an effect of improving the efficiency and/or the lifespan of the organic electroluminescent device.

In the organic electroluminescent device of the present disclosure, preferably, at least one layer (hereinafter, “a surface layer”) selected from a chalcogenide layer, a halogenated metal layer, and a metal oxide layer may be placed on an inner surface(s) of one or both of a pair of electrodes. Specifically, a chalcogenide (including oxides) layer of silicon and aluminum is preferably placed on an anode surface of an electroluminescent medium layer, and a halogenated metal layer or a metal oxide layer is preferably placed on a cathode surface of an electroluminescent medium layer. The operation stability for the organic electroluminescent device may be obtained by the surface layer. Preferably, the chalcogenide includes SiO_{X}(1≤X≤2), AlO_{X}(1≤X≤1.5), SiON, SiAlON, etc.; the halogenated metal includes LiF, MgF₂, CaF₂, a rare earth metal fluoride, etc.; and the metal oxide includes Cs₂O, Li₂O, MgO, SrO, BaO, CaO, etc.

In addition, in the organic electroluminescent device of the present disclosure, a mixed region of an electron transport compound and a reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to an electroluminescent medium. Furthermore, the hole transport compound is oxidized to a cation, and thus it becomes easier to inject and transport holes from the mixed region to the electroluminescent medium. Preferably, the oxidative dopant includes various Lewis acids and acceptor compounds, and the reductive dopant includes alkali metals, alkali metal compounds, alkaline earth metals, rare-earth metals, and mixtures thereof. Also, a reductive dopant layer may be employed as a charge generating layer to prepare an organic electroluminescent device having two or more light-emitting layers and emitting white light.

An organic electroluminescent device according to one embodiment may further include one or more dopants in the light-emitting layer.

The dopant comprised in the organic electroluminescent device of the present disclosure may be at least one phosphorescent or fluorescent dopant, preferably a phosphorescent dopant. The phosphorescent dopant material applied to the organic electroluminescent device of the present disclosure is not particularly limited, but may be preferably a metallated complex compound(s) of a metal atom(s) selected from iridium (Ir), osmium (Os), copper (Cu), and platinum (Pt), more preferably an ortho-metallated complex compound(s) of a metal atom(s) selected from iridium (Ir), osmium (Os), copper (Cu), and platinum (Pt), and even more preferably ortho-metallated iridium complex compound(s).

The dopant comprised in the organic electroluminescent device of the present disclosure may use the compound represented by the following Formula 101, but is not limited thereto.

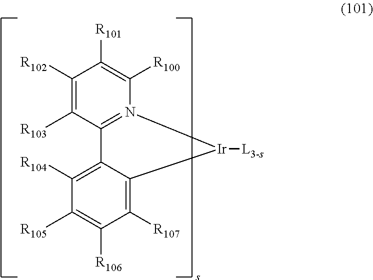

in Formula 101,
L is selected from any one of the following structures 1 to 3;

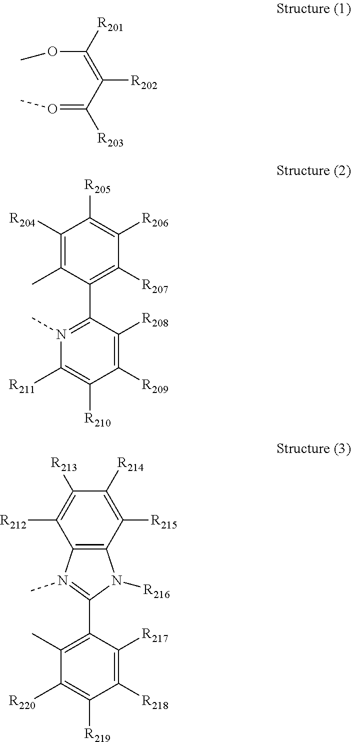

wherein,
R₁₀₀ to R₁₀₃ each independently represent, hydrogen, deuterium, halogen, (C1-C30)alkyl unsubstituted or substituted with deuterium and/or halogen, a substituted or unsubstituted (C3-C30)cycloalkyl, a substituted or unsubstituted (C6-C30)aryl, cyano, a substituted or unsubstituted (C3-C30) heteroaryl, or a substituted or unsubstituted (C1-C30)alkoxy; or R₁₀₀ to R₁₀₃ may be linked to the adjacent substituents to form a ring(s), for example, to form a ring(s) with a benzene, e.g., a substituted or unsubstituted quinoline, a substituted or unsubstituted benzofuropyridine, a substituted or unsubstituted benzothienopyridine, a substituted or unsubstituted indenopyridine, a substituted or unsubstituted benzofuroquinoline, a substituted or unsubstituted benzothienoquinoline, or a substituted or unsubstituted indenoquinoline;
R₁₀₄ to R₁₀₇ each independently represent, hydrogen, deuterium, halogen, (C1-C30)alkyl unsubstituted or substituted with deuterium and/or halogen, a substituted or unsubstituted (C3-C30)cycloalkyl, a substituted or unsubstituted (C6-C30)aryl, a substituted or unsubstituted (C3-C30)heteroaryl, cyano, or a substituted or unsubstituted (C1-C30)alkoxy; or R₁₀₄ to R₁₀₇ may be linked to the adjacent substituents to form a substituted or unsubstituted ring(s), for example to form a substituted or unsubstituted ring(s) with a benzene, e.g., a substituted or unsubstituted naphthalene, a substituted or unsubstituted fluorene, a substituted or unsubstituted dibenzothiophene, a substituted or unsubstituted dibenzofuran, a substituted or unsubstituted indenopyridine, a substituted or unsubstituted benzofuropyridine, or a substituted or unsubstituted benzothienopyridine;
R₂₀₁ to R₂₂₀ each independently represent hydrogen, deuterium, halogen, (C1-C30)alkyl unsubstituted or substituted with deuterium and/or halogen, a substituted or unsubstituted (C3-C30)cycloalkyl, or a substituted or unsubstituted (C6-C30)aryl; or R₂₀₁ to R₂₂₀ may be linked to an adjacent substituent(s) to form a substituted or unsubstituted ring(s); and
s represents an integer of 1 to 3.

Specifically, the specific examples of the dopant compound include the following, but are not limited thereto.

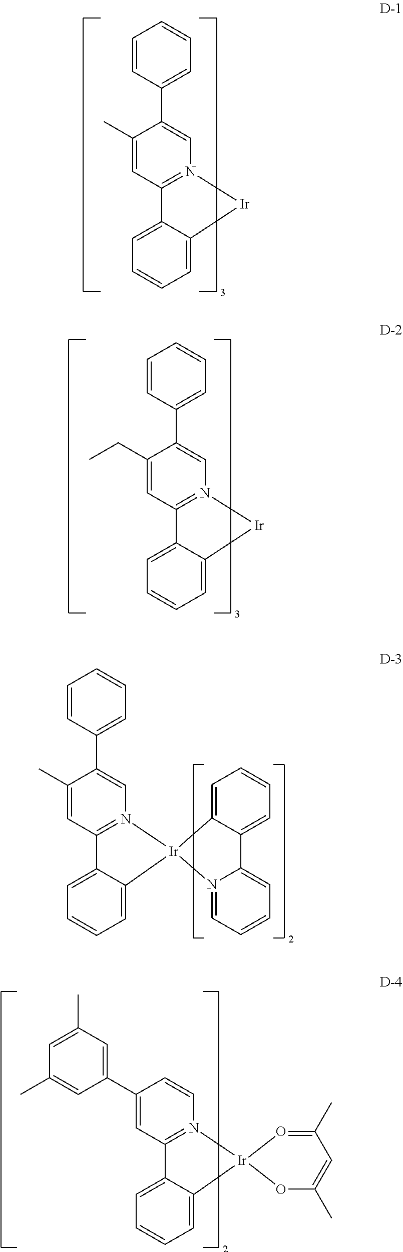

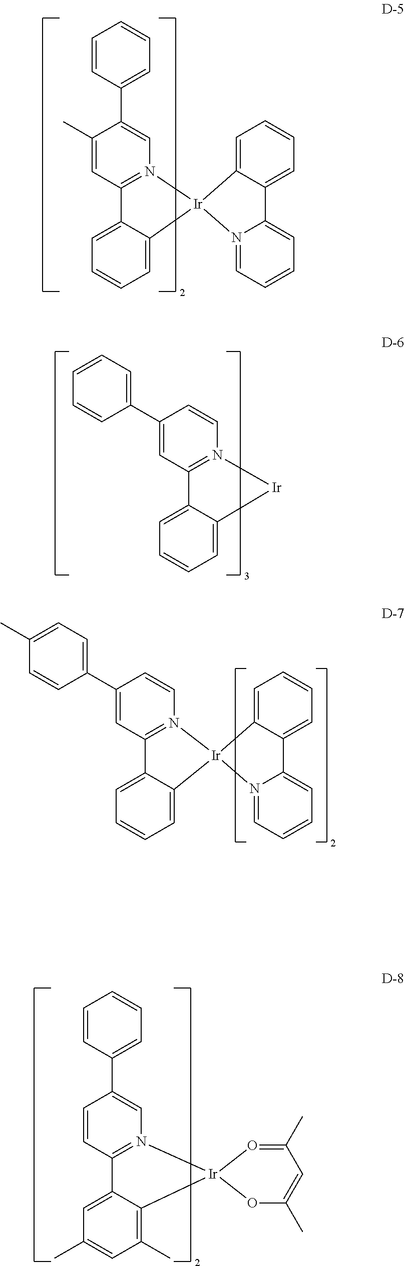

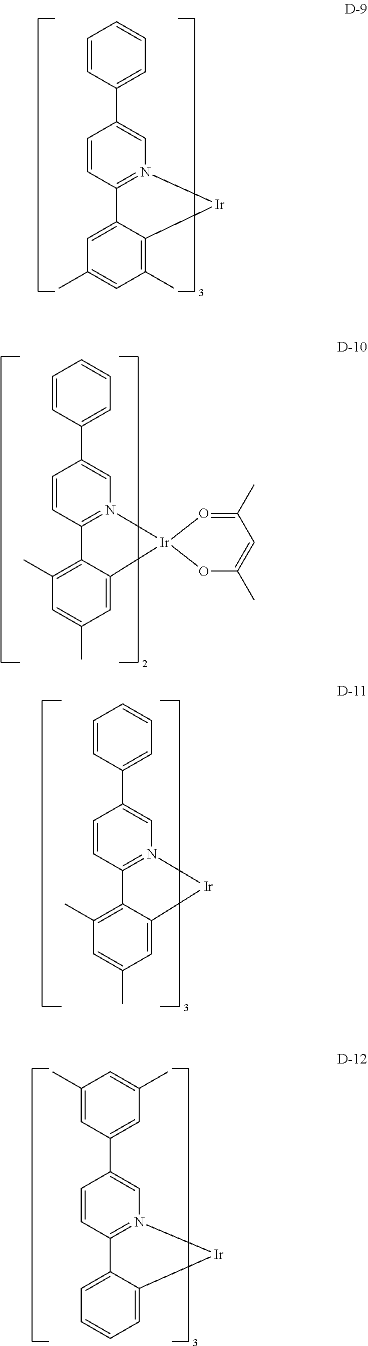

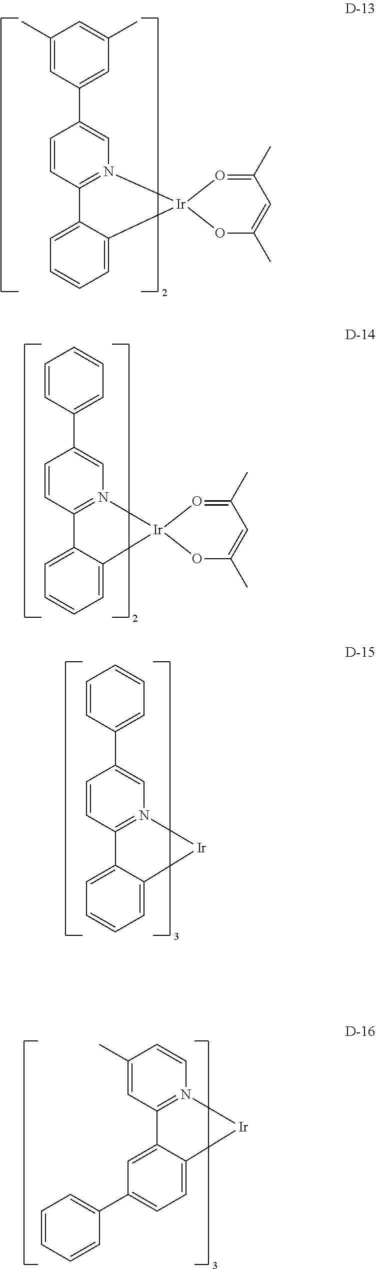

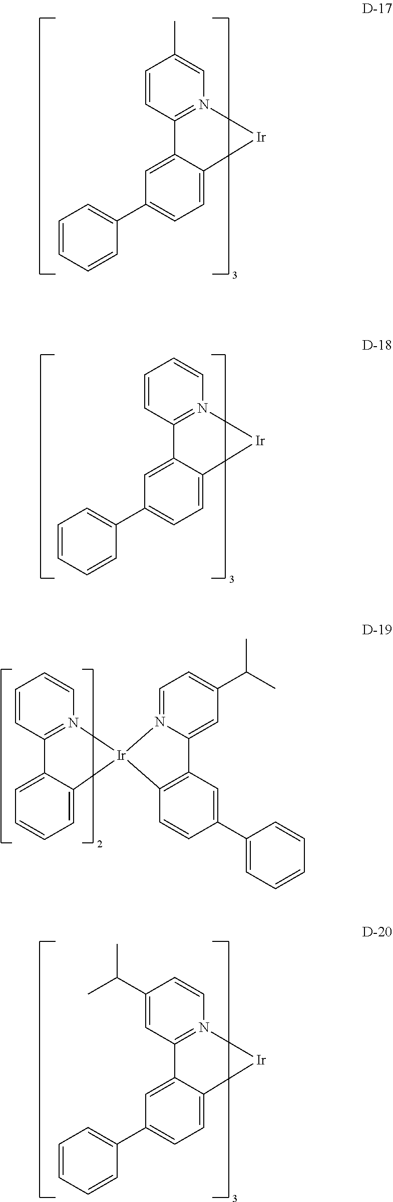

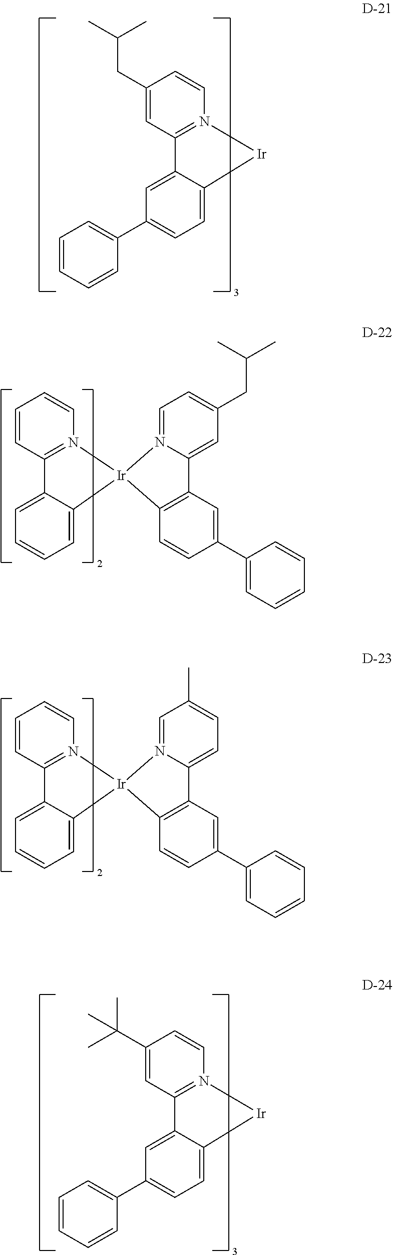

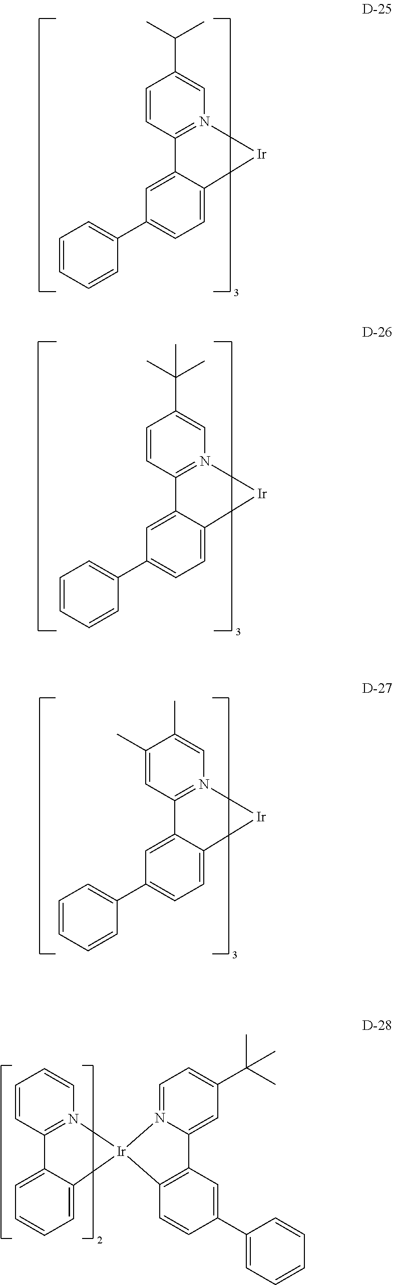

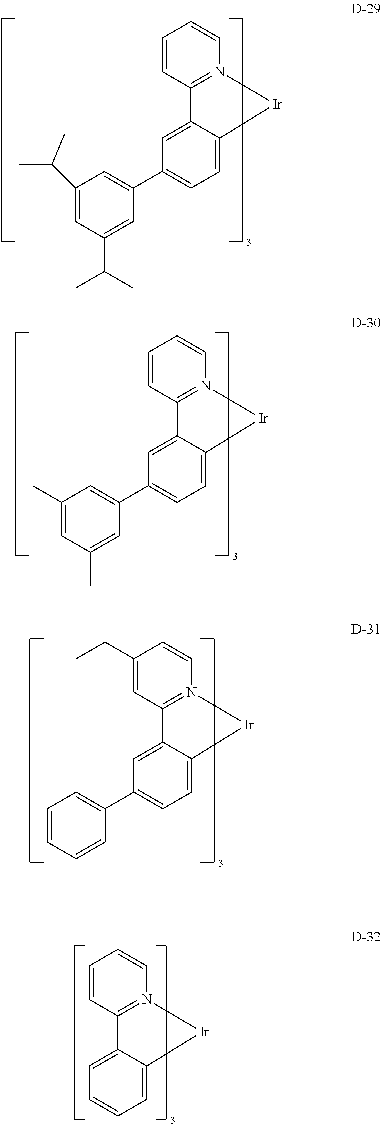

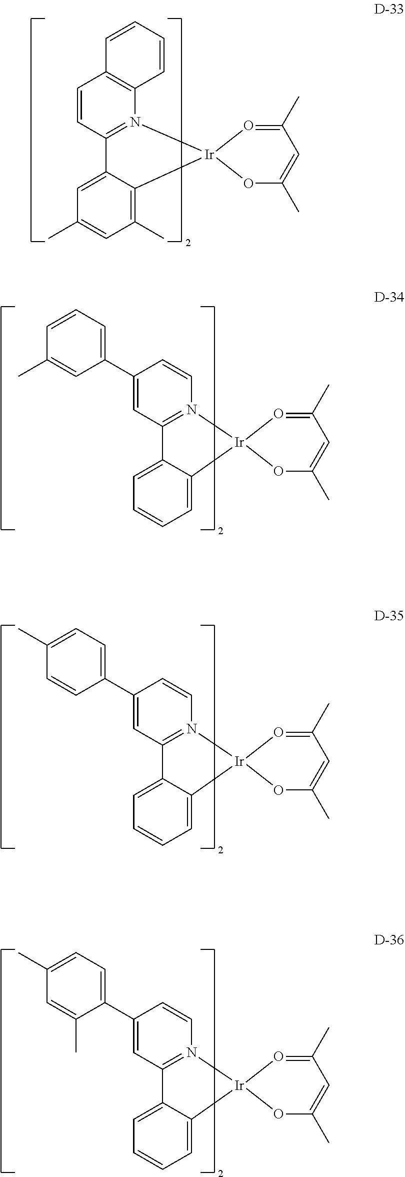

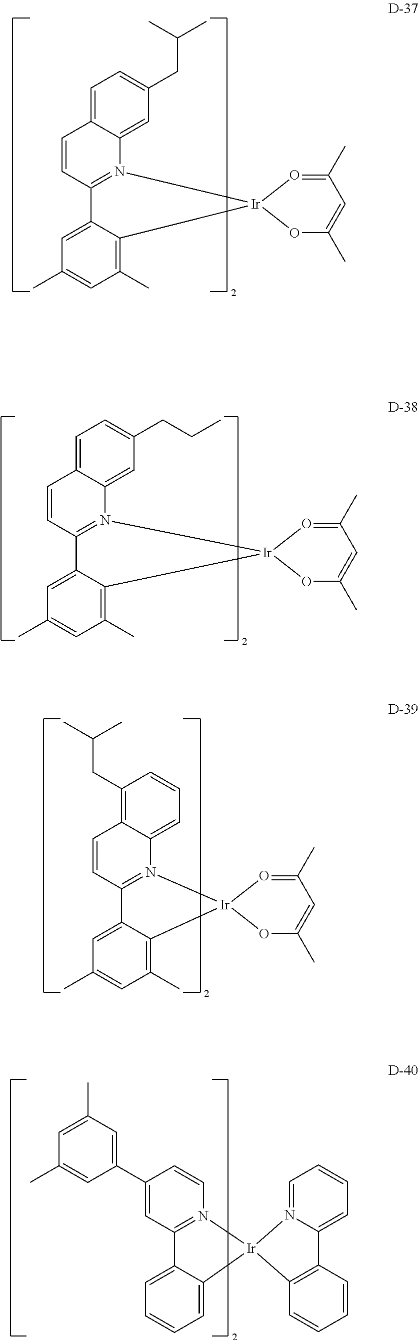

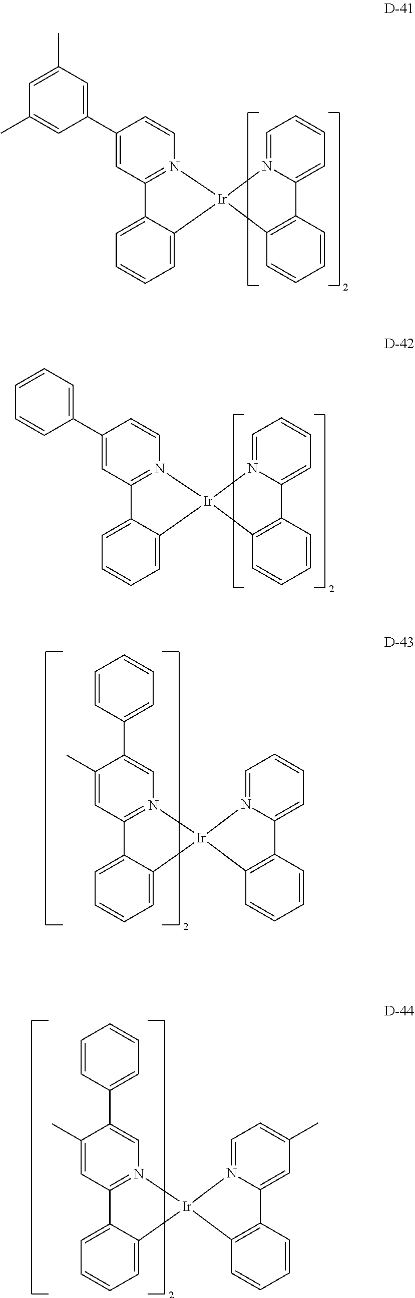

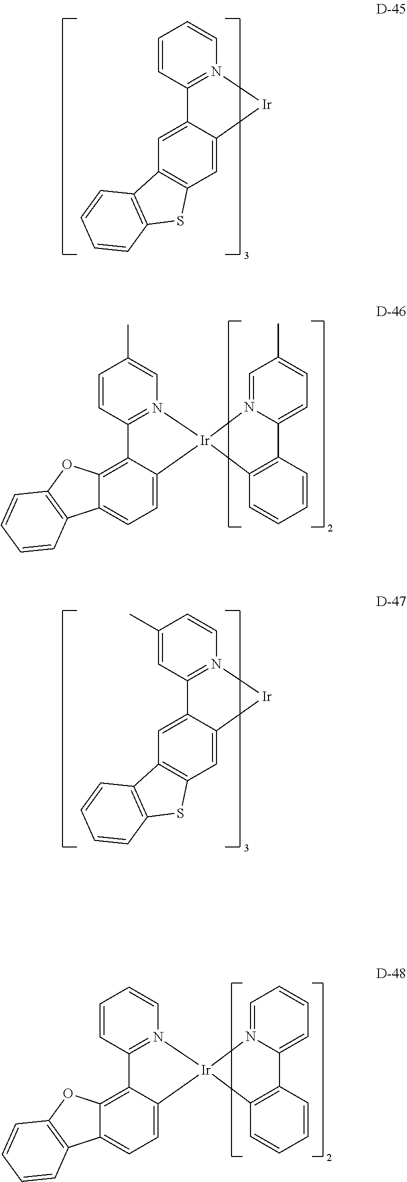

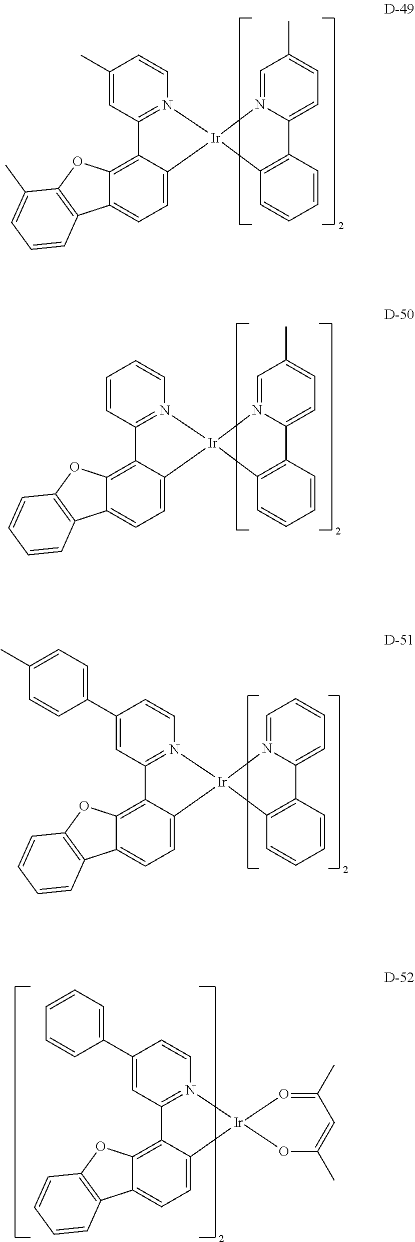

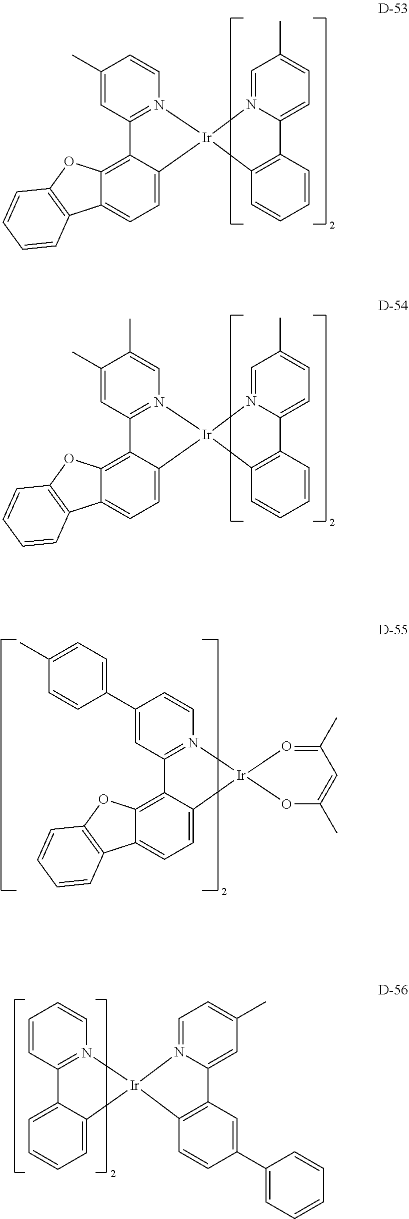

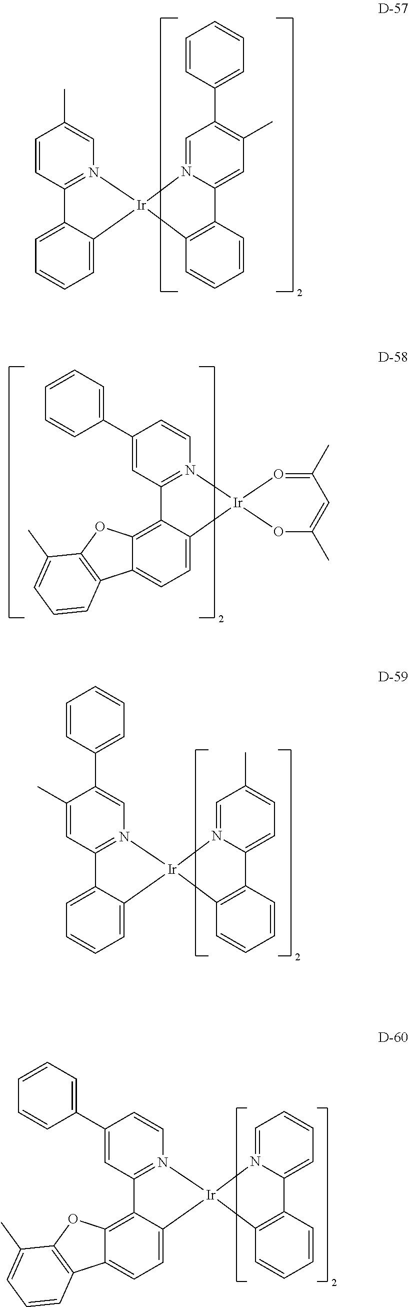

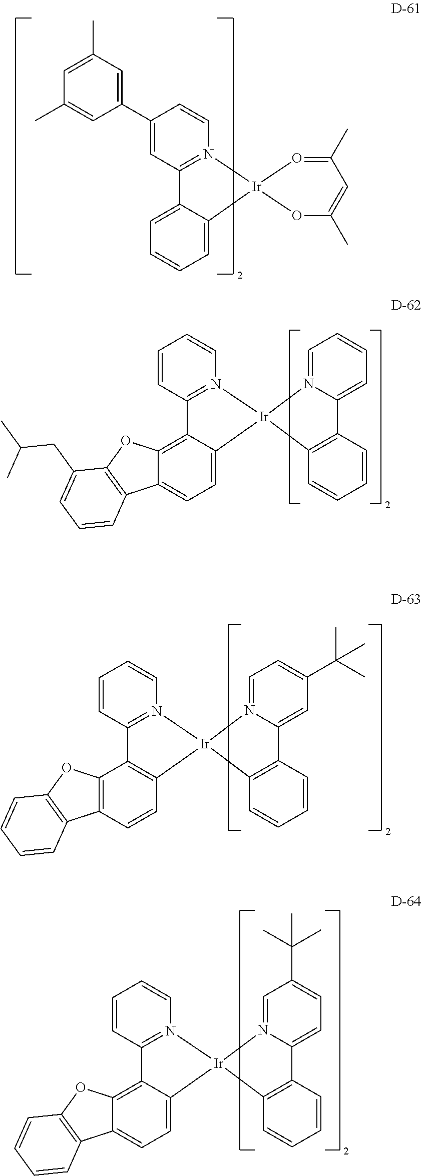

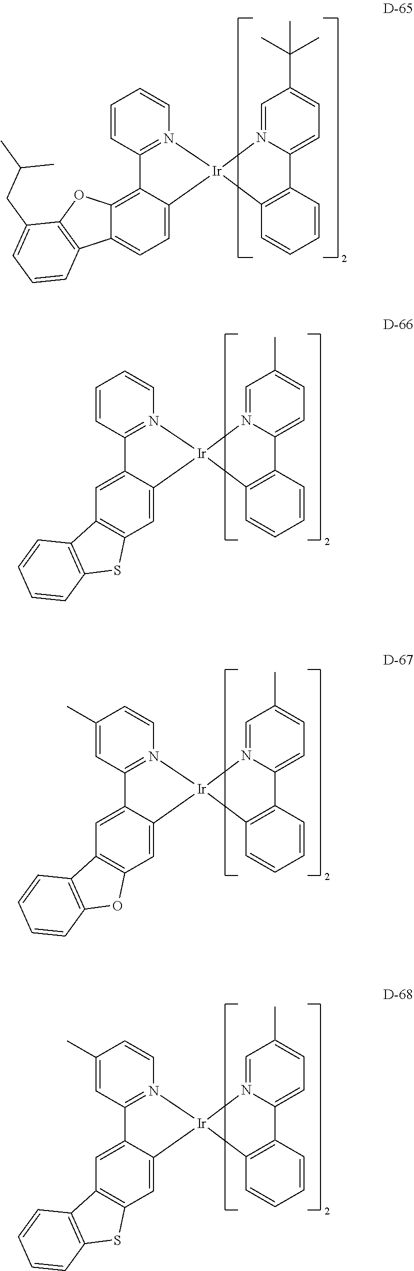

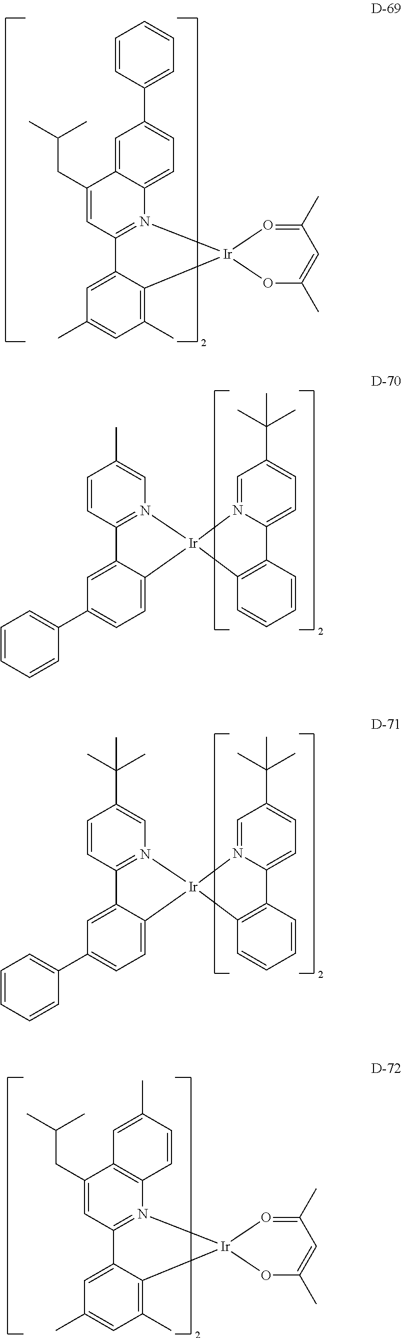

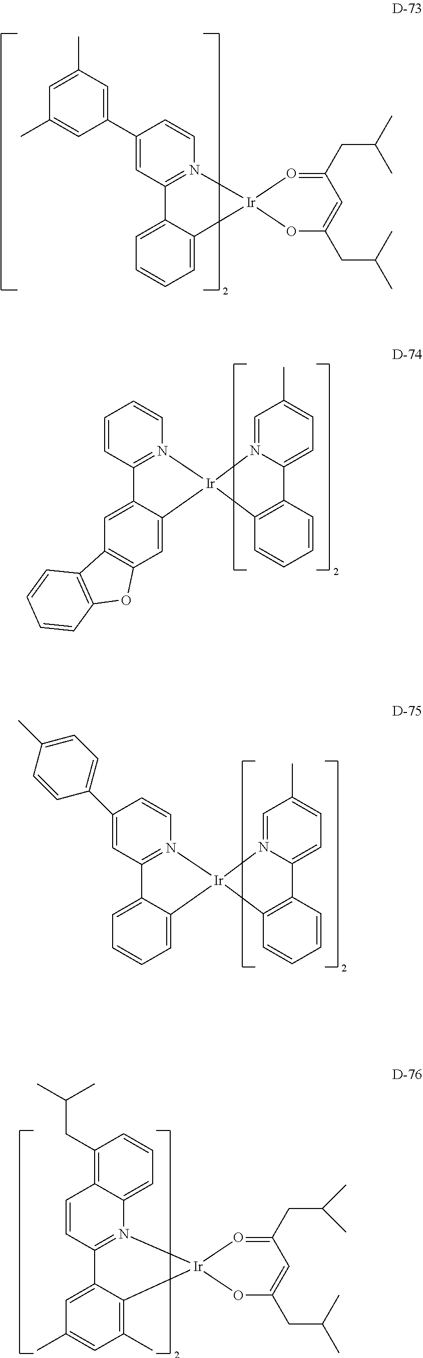

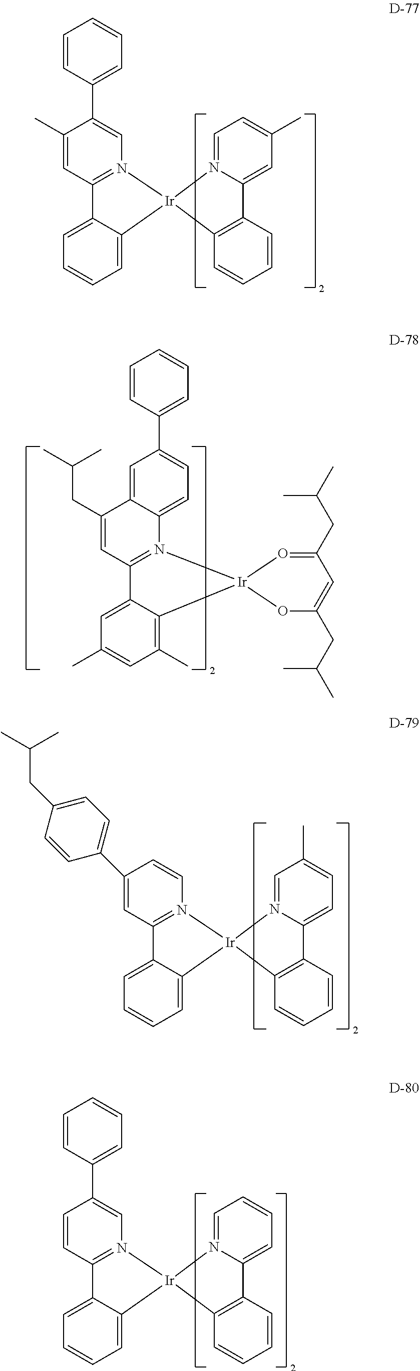

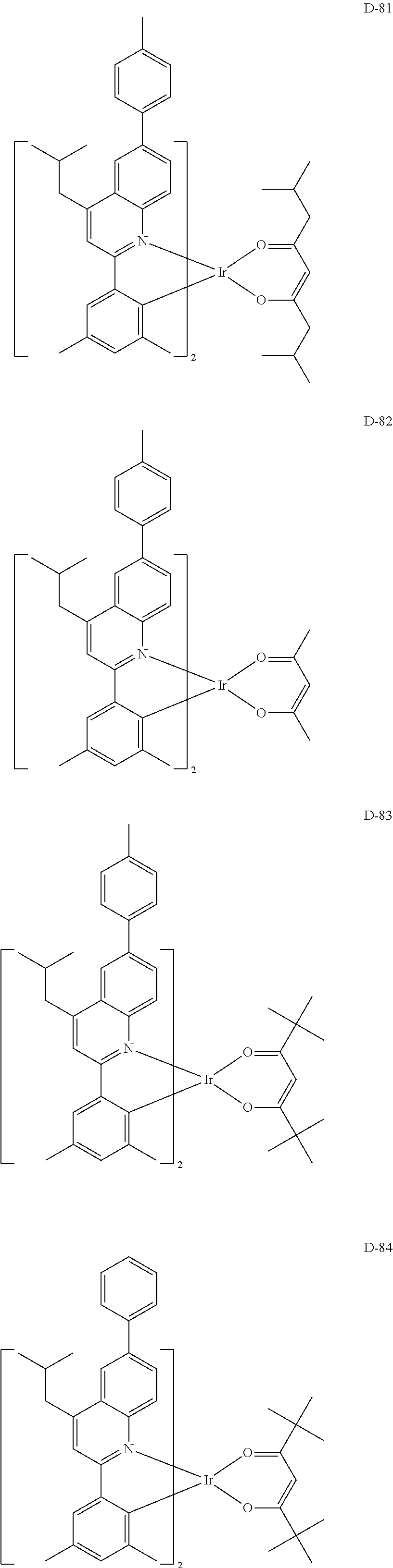

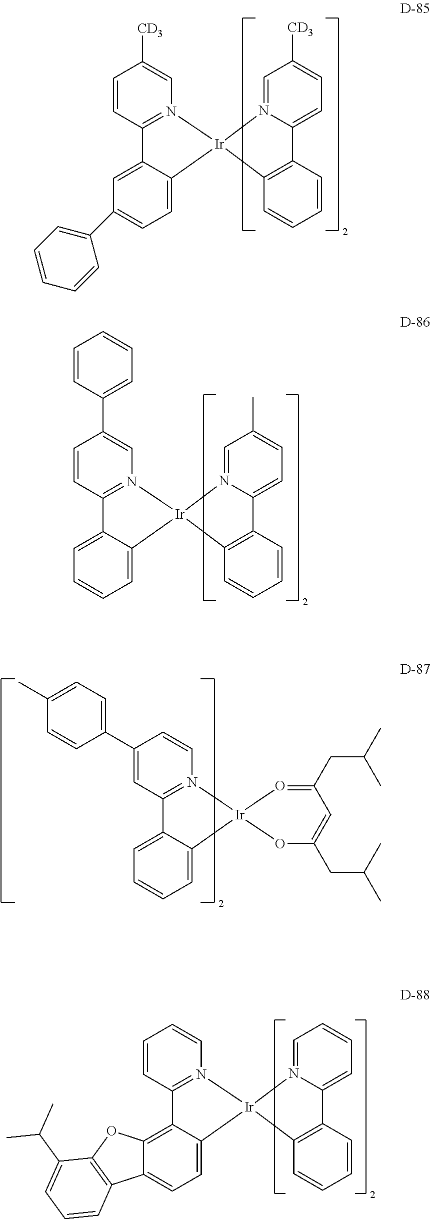

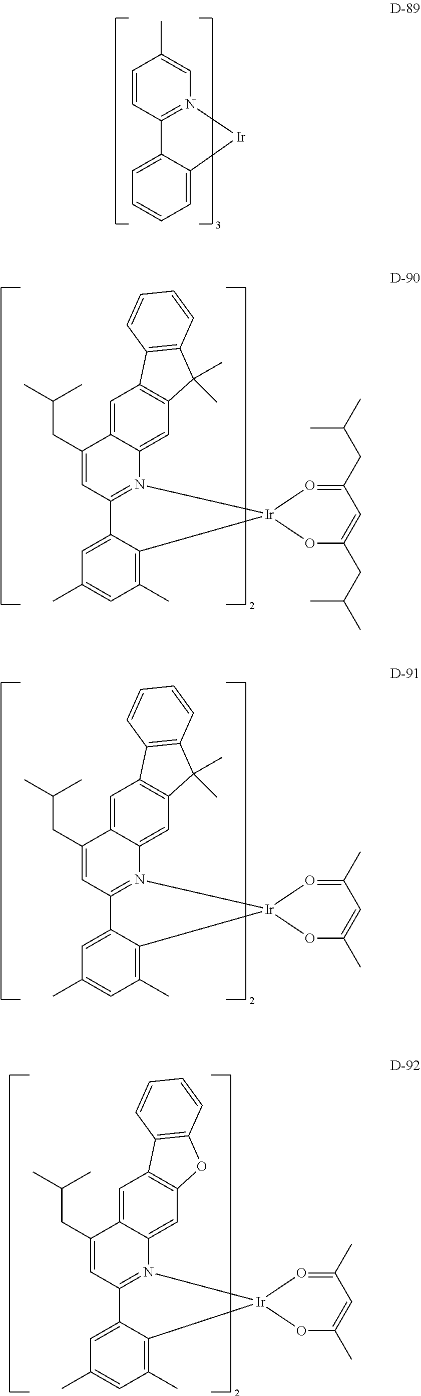

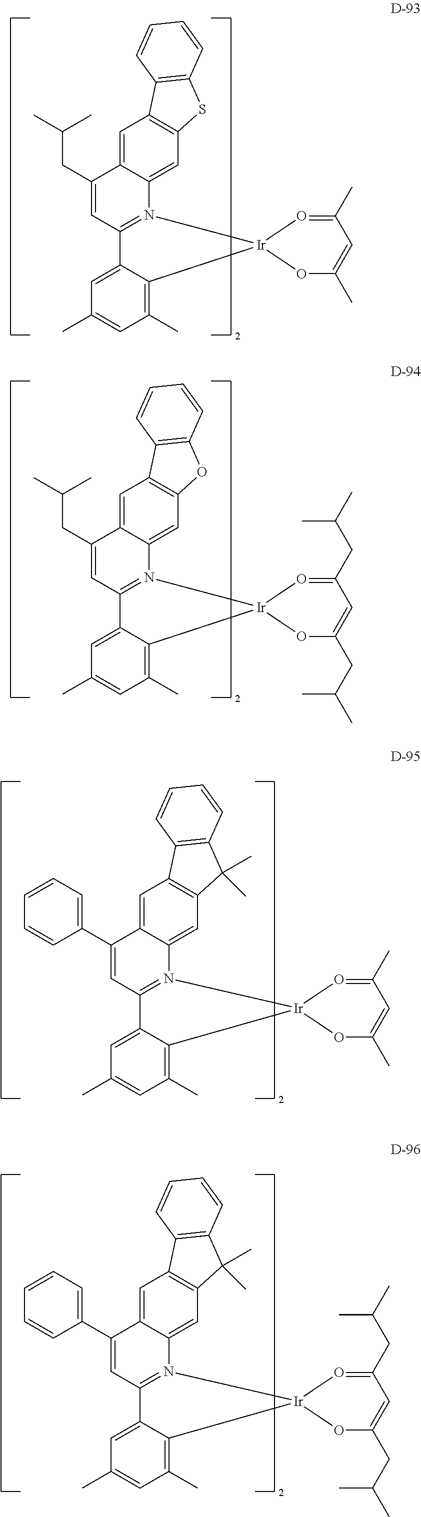

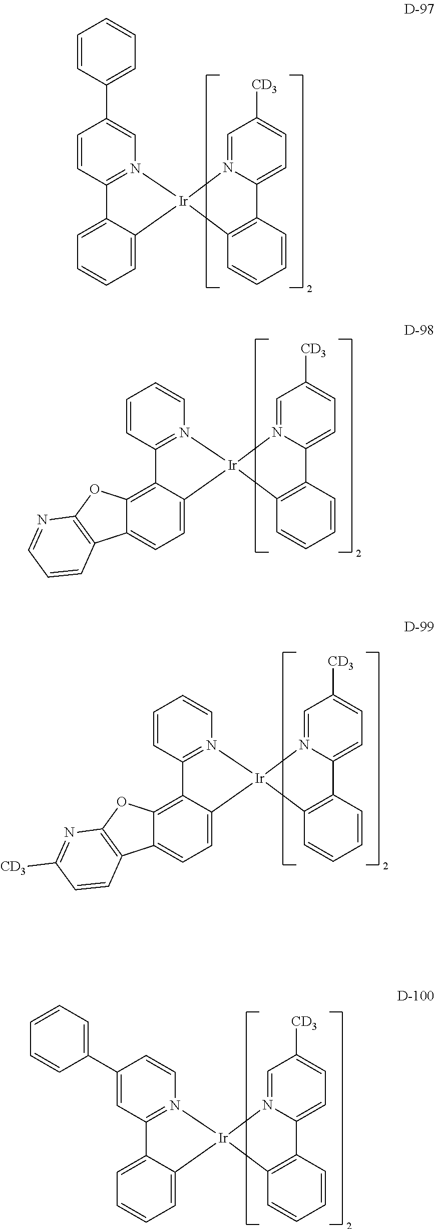

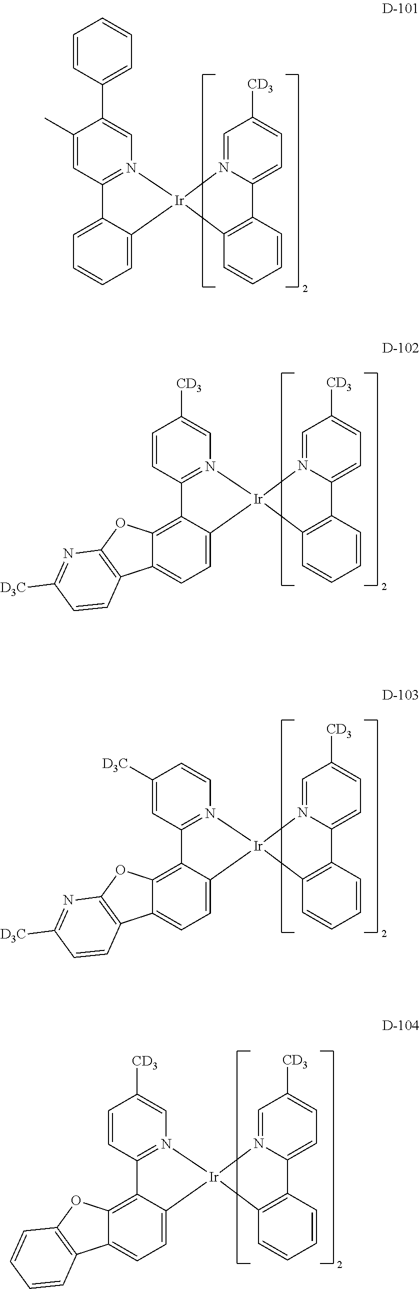

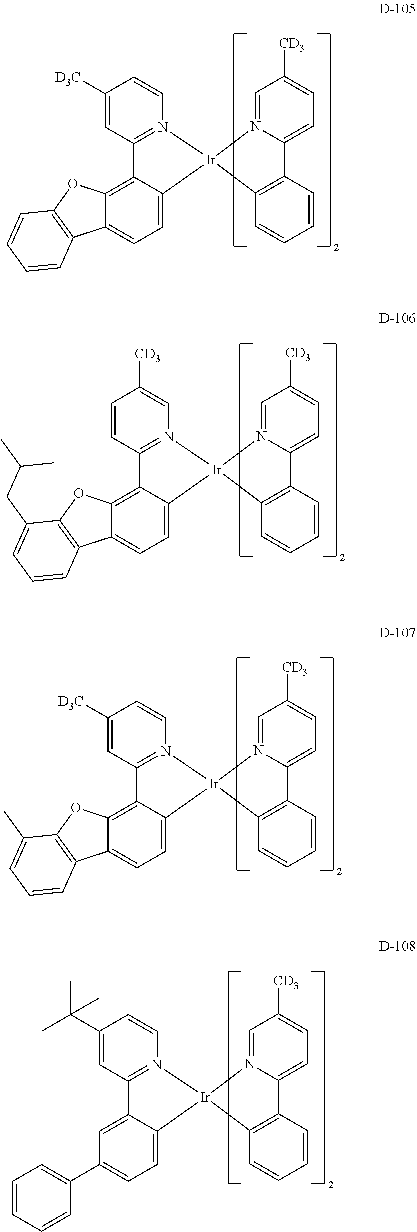

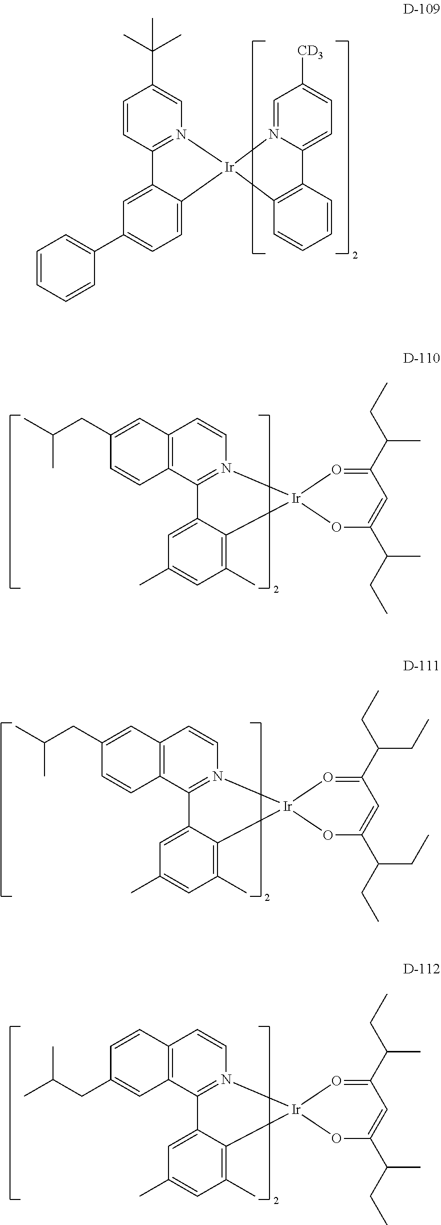

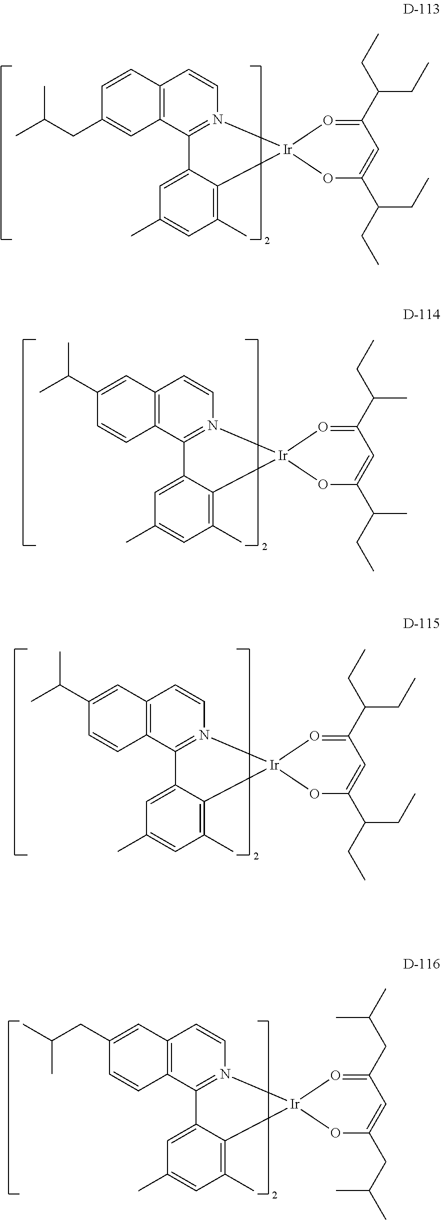

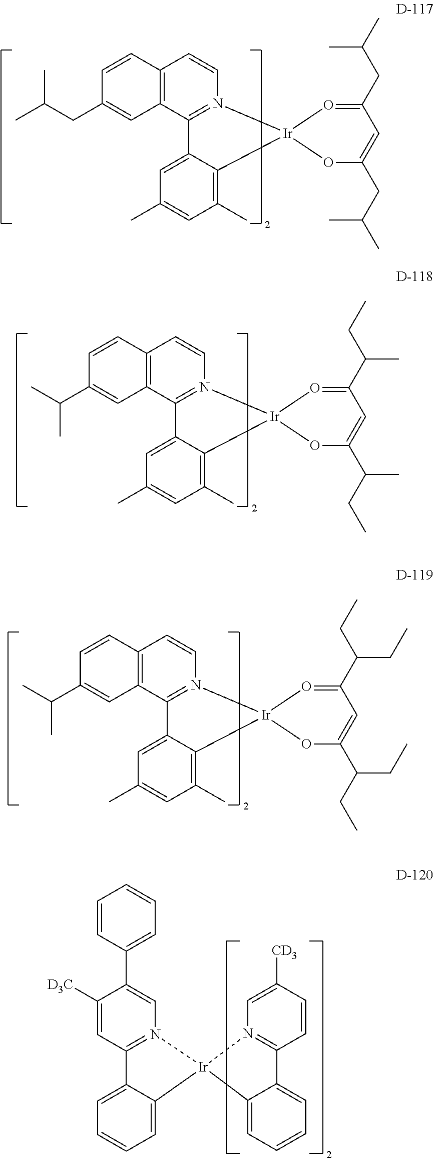

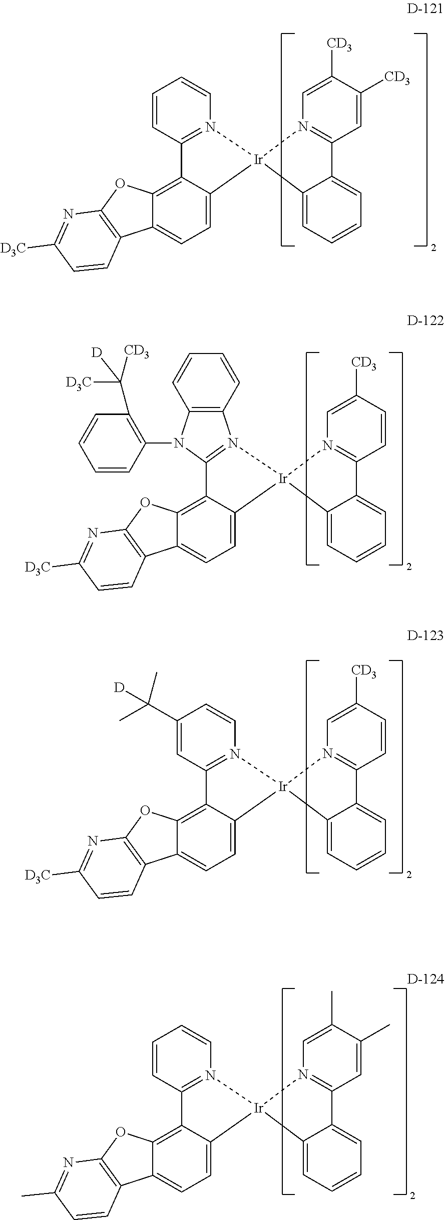

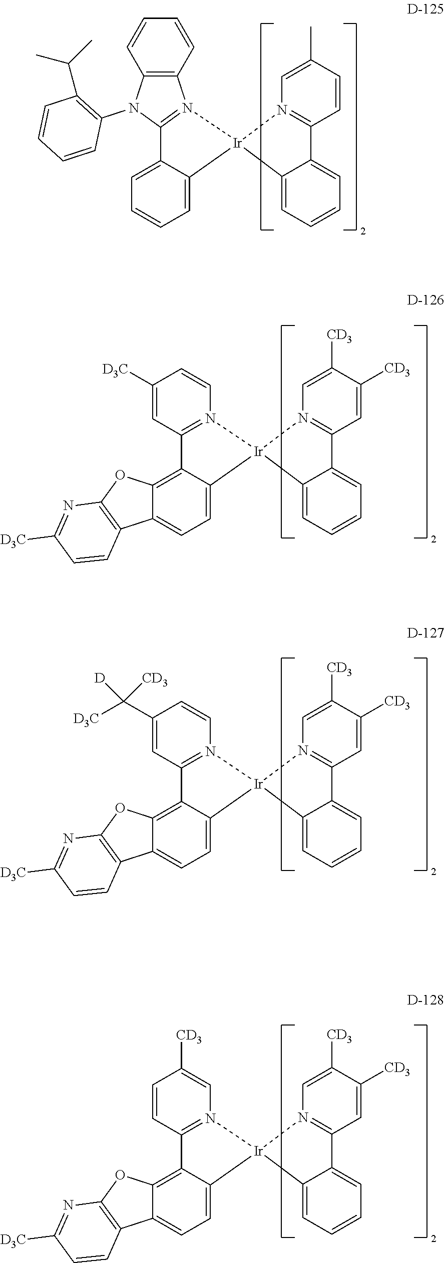

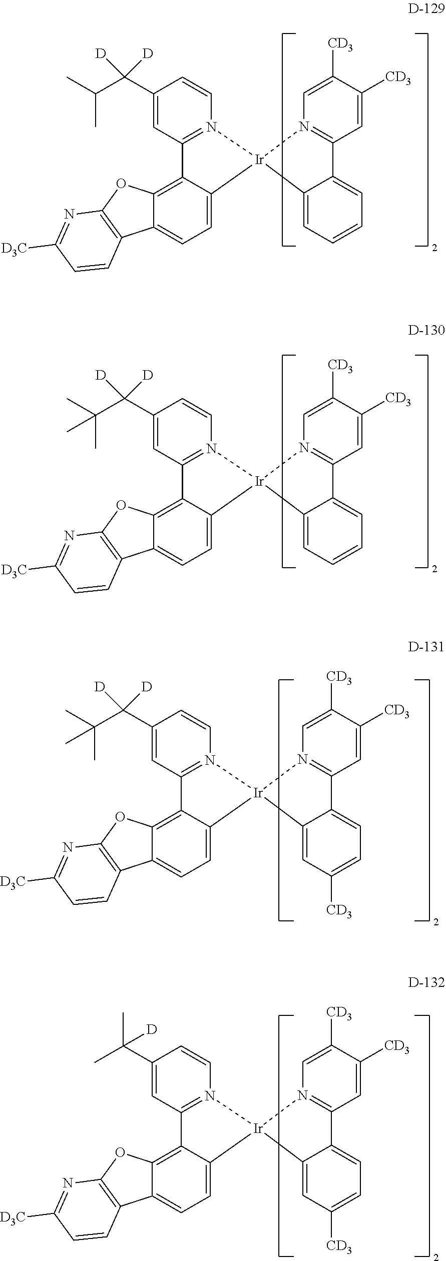

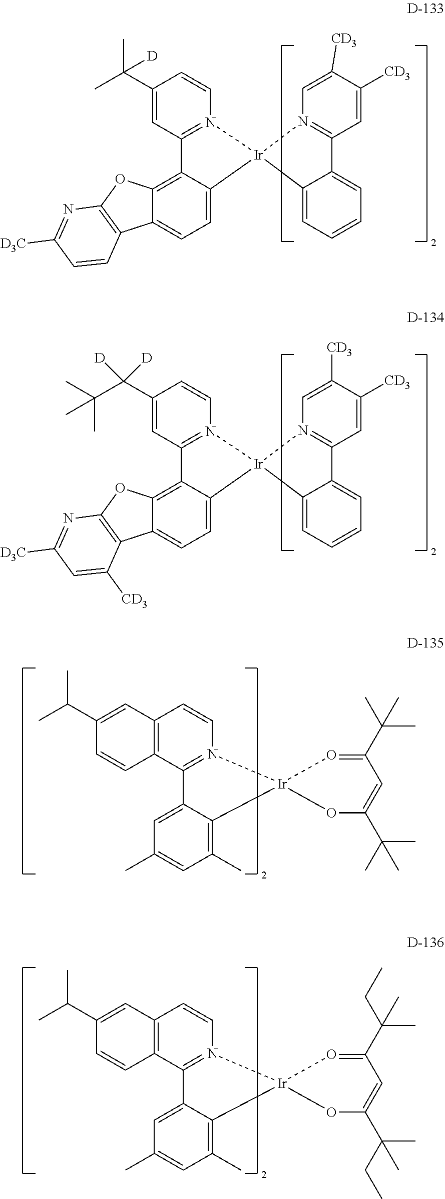

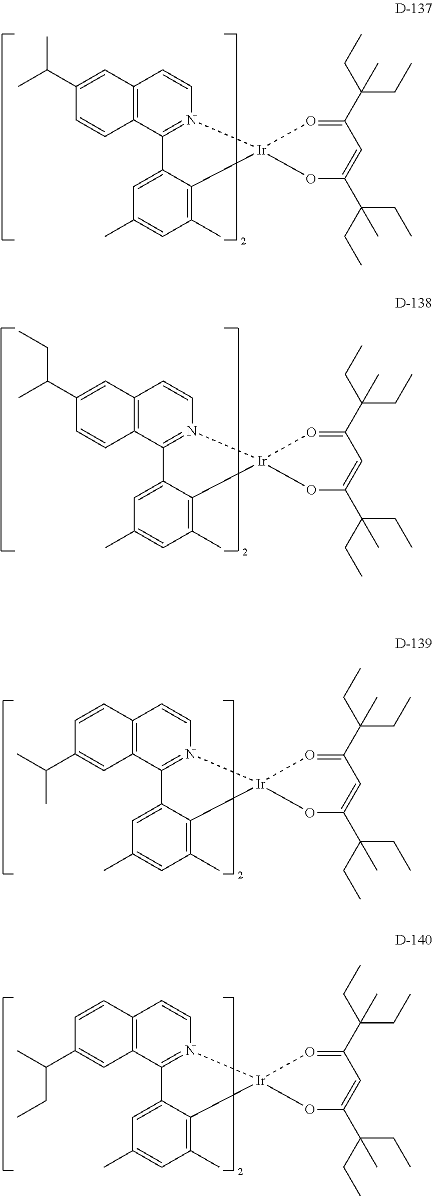

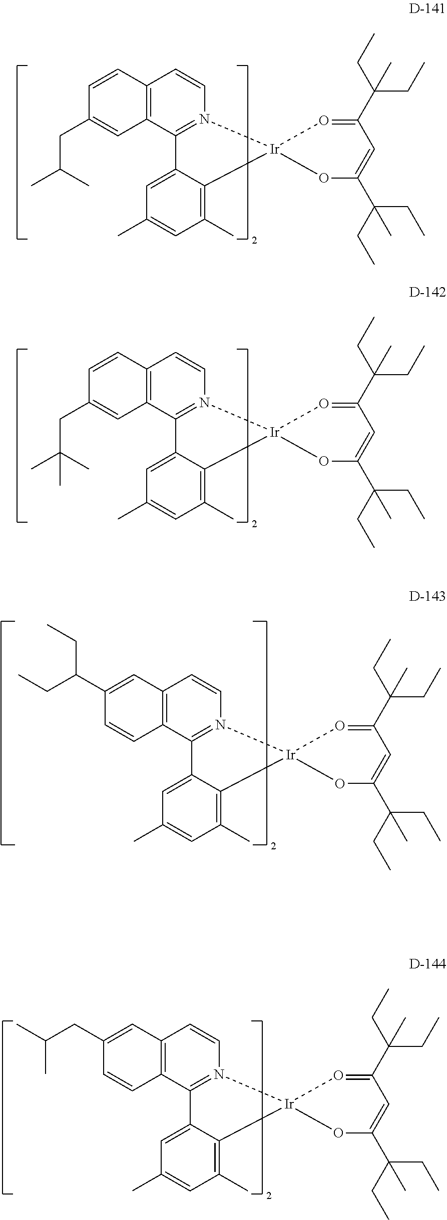

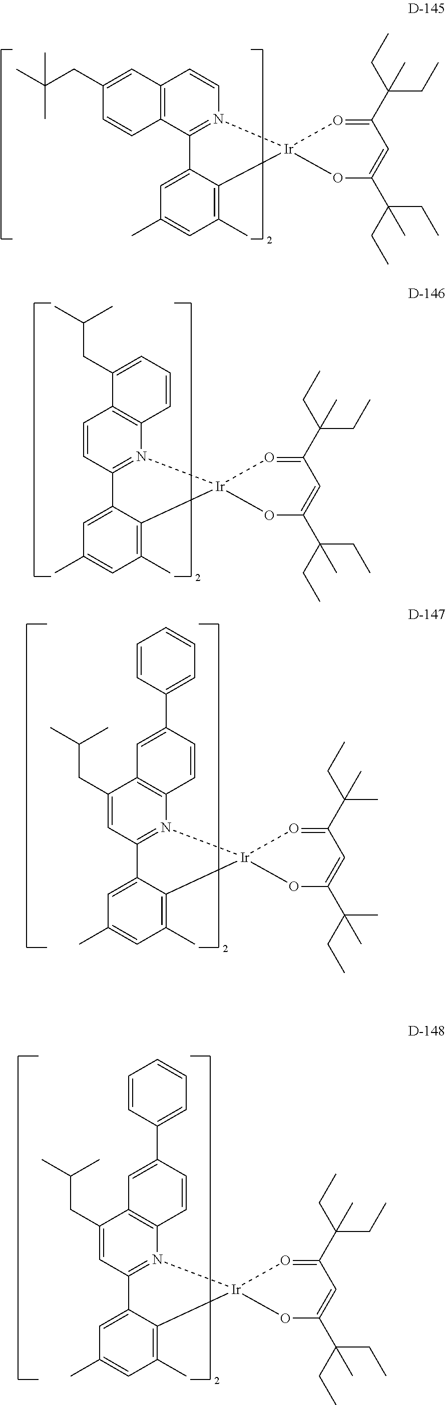

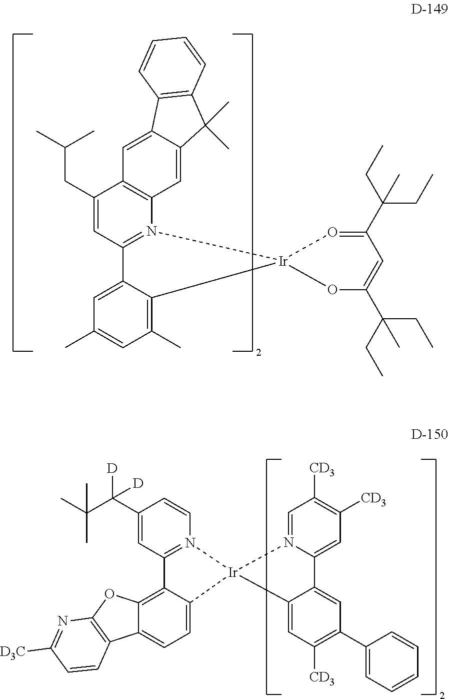

The compound represented by Formula 1, 11, or 12 according to the present disclosure may be produced by a synthetic method known to a person skilled in the art, for example, they may be prepared with reference to the following reaction schemes 1-1 to 1-3, but is not limited thereto.

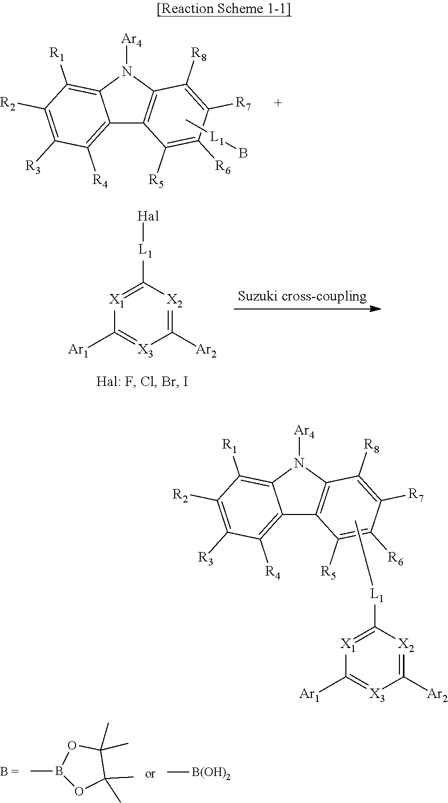

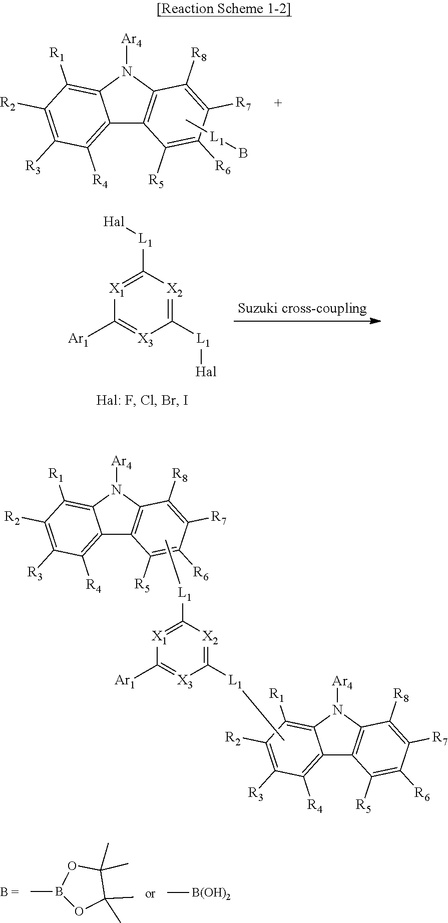

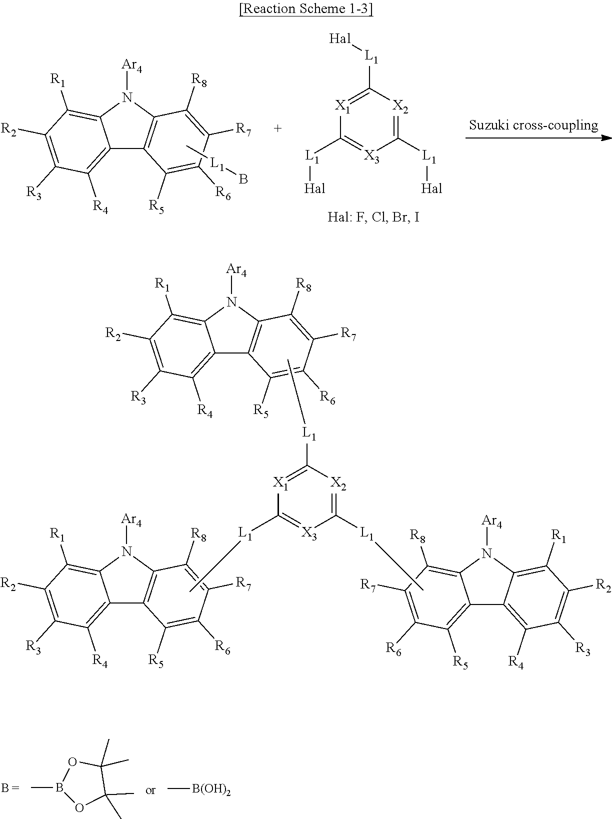

In reaction schemes 1-1 to 1-3, X₁ to X₃, R₁ to R₈, L₁, Ar₁, Ar₂, and Ar₄ each independently represent as defined in Formula 1.

The compound represented by Formula 2 or 13 according to the present disclosure may be produced by a synthetic method known to a person skilled in the art. For example, the compound represented by Formula 2 may be prepared with reference to the following reaction scheme 2, but is not limited thereto.

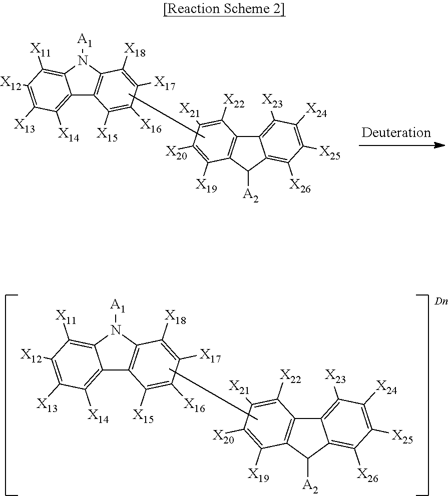

In Reaction Scheme 2, A₁, A₂, and X₁₁ to X₂₆ each independently represent as defined in Formula 2, Dn means that n number of hydrogens is replaced with deuterium, wherein n represents an integer of 1 or more and the upper limit of n is determined by the number of hydrogens that can be substituted in each compound.

As described above, exemplary synthesis examples of the compounds represented by Formula 1, 2, 11, 12 or 13 are described, but they are based on Buchwald-Hartwig cross coupling reaction, N-arylation reaction, H-mont-mediated etherification reaction, Miyaura borylation reaction, Suzuki cross-coupling reaction, Wittig reaction, Intramolecular acid-induced cyclization reaction, Pd(II)-catalyzed oxidative cyclization reaction, Grignard reaction, Heck reaction, Cyclic Dehydration reaction, SN₁ substitution reaction, SN₂ substitution reaction, and Phosphine-mediated reductive cyclization reaction etc. It will be understood by one skilled in the art that the above reaction proceeds even if other substituents defined in the Formula 1, 2, 11, 12, or 13 other than the substituents described in the specific synthesis examples are bonded.

In addition, the deuteriumized compound of formulas 1, 2, and 11 can be prepared using a deuteriumized precursor material in a similar manner, or more generally can be prepared by treating a non-deuteriumized compound with a deuteriumized solvent, D6-benzene in the presence of a Lewis acid H/D exchange catalyst such as aluminum trichloride or ethyl aluminum chloride. In addition, the degree of deuteriumization can be controlled by varying reaction conditions such as reaction temperature. For example, the number of deuterium in formulas 1, 2, and 11 can be adjusted by controlling the reaction temperature and time, the equivalent of acid, etc.

In order to form each layer of the organic electroluminescent device of the present disclosure, dry film-forming methods such as vacuum evaporation, sputtering, plasma, ion plating methods, etc., or wet film-forming methods such as ink jet printing, nozzle printing, slot coating, spin coating, dip coating, flow coating methods, etc., can be used. When forming a film by the organic electroluminescent compound of the present application or the first host compound and the second host compound, co-deposition or mixed-deposition is performed.

When using a wet film-forming method, a thin film may be formed by dissolving or diffusing materials forming each layer into any suitable solvent such as ethanol, chloroform, tetrahydrofuran, dioxane, etc. The solvent may be any solvent where the materials forming each layer can be dissolved or diffused, and where there are no problems in film-formation capability.

In addition, by using the organic electroluminescent device of the present disclosure, display devices such as smartphones, tablets, notebooks, PCs, TVs, or display devices for vehicles, or lighting devices such as outdoor or indoor lighting can be prepared.

Hereinafter, the preparation method of the compounds according to the present disclosure and the physical properties thereof, and light-emitting properties of an OLED comprising an organic electroluminescent compound or a plurality of host materials according to the present disclosure will be explained with reference to the synthesis method of a representative compound in order to understand the present disclosure in detail. However, the following examples are only for describing the properties of an OLED including an organic electroluminescent compound or a plurality of host materials according to the present disclosure in order to understand the present disclosure in detail, but the present disclosure is not limited to the following examples.

### [Example 1] Preparation of Compound C-52

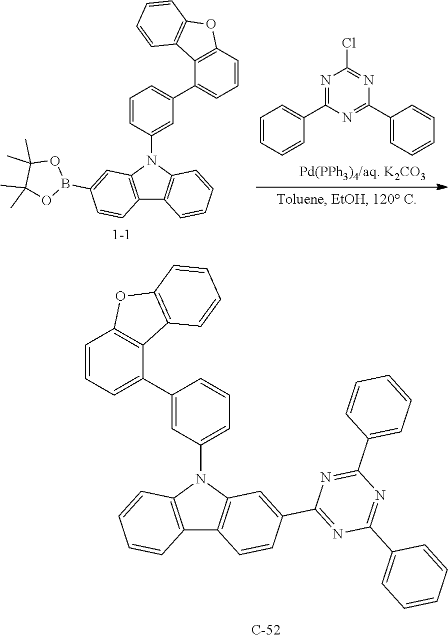

Compound 1-1 (7.0 g, 13.07 mmol), 2-chloro-4,6-diphenyl-1,3,5-triazine (3.8 g, 14.38 mmol), tetrakis(triphenylphosphine)palladium(0) (0.5 g, 0.39 mmol), potassium carbonate (4.5 g, 32.68 mmol), 65 mL of toluene, 16 mL of ethanol, and 16 mL of distilled water were added to a flask and dissolved, followed by stirred under reflux at 120° C. for 4 hours. At the end of the reaction, the organic layer was extracted with ethyl acetate and then separated by column chromatography to obtain Compound C-52 (3.2 g, yield: 38%).

| Compound | MW | M.P. |
|---|---|---|
| C-52 | 640.73 | 224° C. |

### [Example 2] Preparation of Compound C-112

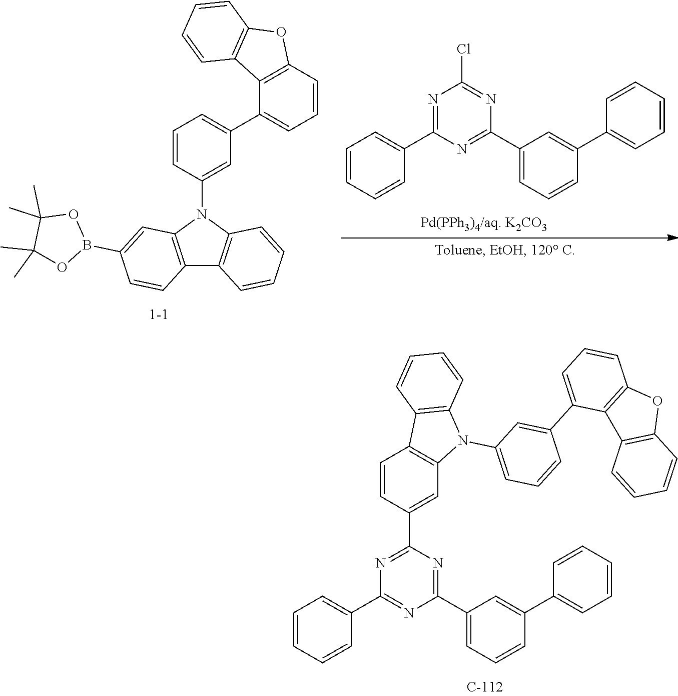

Compound 1-1 (4.6 g, 8.59 mmol), 2-([1,1′-biphenyl]-3-yl)-4-chloro-6-phenyl-1,3,5-triazine (3.3 g, 9.45 mmol), tetrakis(triphenylphosphine)palladium(0) (0.3 g, 0.26 mmol), potassium carbonate (3.0 g, 21.48 mmol), 43 mL of toluene, 10 mL of ethanol, and 10 mL of distilled water were added to a flask and dissolved, followed by stirred under reflux at 120° C. for 4 hours. After the reaction was completed, the organic layer was extracted with ethyl acetate and then separated by column chromatography to obtain Compound C-112 (2.0 g, yield: 33%).

| Compound | MW | M.P. |
|---|---|---|
| C-112 | 716.83 | 265° C. |

### [Example 3] Preparation of Compound C-2

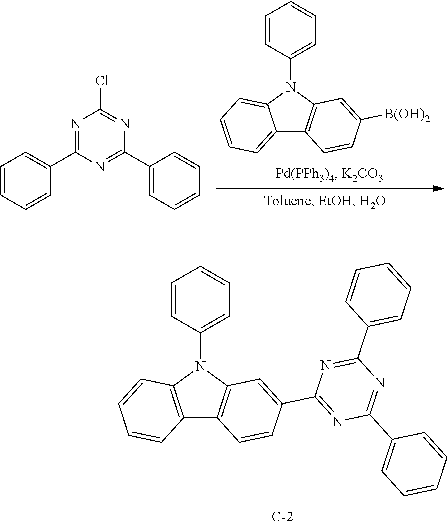

2-chloro-4,6-diphenyl-1,3,5-triazine (15 g, 56.0 mmol), (9-phenyl-9H-carbazol-2-yl)boronic acid (19.3 g, 19.3 mmol), tetrakis(triphenylphosphine)palladium(0) (3.2 g, 2.8 mmol), potassium carbonate (19.4 g, 140.0 mmol), 260 mL toluene, 65 mL ethanol, and 65 mL water were added to a flask and stirred at 120° C. for 2 hours. After the reaction was completed, the organic layer was extracted with ethyl acetate, the residual moisture was removed using magnesium sulfate, dried, and separated by column chromatography to obtain Compound C-2 (13.6 g, yield: 51%).

| Compound | MW | M.P |
|---|---|---|
| C-2 | 474.57 | 244° C. |

### [Example 4] Preparation of Compound C-152

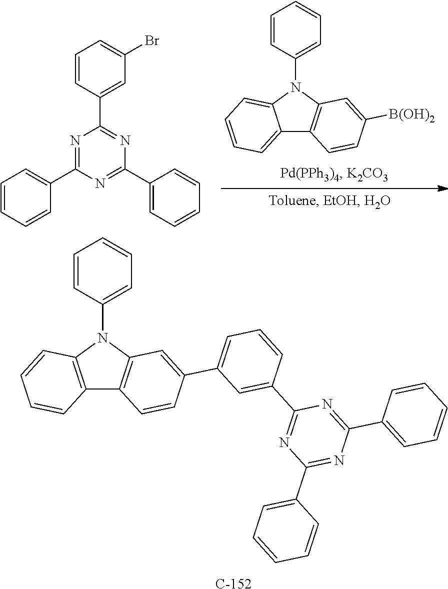 [SMILES: Brc1cccc(-c2nc(-c3ccccc3)nc(-c3ccccc3)n2)c1 ; OB(O)c1ccc2c3ccccc3n(-c3ccccc3)c2c1 ; c1ccc(-c2nc(-c3ccccc3)nc(-c3cccc(-c4ccc5c6ccccc6n(-c6ccccc6)c5c4)c3)n2)cc1 ; "C-152"]

2-(3-Bromophenyl)-4,6-diphenyl-1,3,5-triazine (10 g, 25.7 mmol), (9-phenyl-9H-carbazol-2-yl)boronic acid (8.9 g, 30.9 mmol), tetrakis(triphenylphosphine)palladium(0) (1.5 g, 1.3 mmol), potassium carbonate (9 g, 64.4 mmol), 130 mL of toluene, 32 mL of ethanol, and 32 mL of distilled water were added to a flask, and then stirred at 120° C. for 2 hours. After the reaction was completed, the organic layer was extracted with ethyl acetate and residual moisture was removed using magnesium sulfate and dried. Next, it was separated by column chromatography to obtain Compound C-152 (7.9 g, yield: 56%).

| Compound | MW | M.P. |
|---|---|---|
| C-152 | 550.66 | 178° C. |

### [Example 5] Preparation of Compound C-94

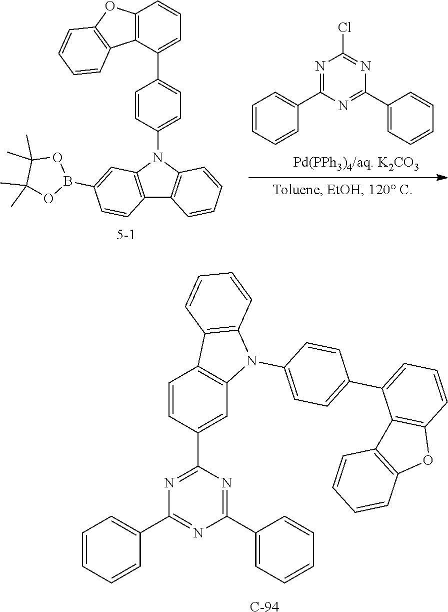

Compound 5-1 (5.9 g, 11.02 mmol), 2-chloro-4,6-diphenyl-1,3,5-triazine (3.0 g, 11.02 mmol), tetrakis(triphenylphosphine)palladium(0) (635 mg, 0.55 mmol), potassium carbonate (4.6 g, 33.06 mmol), 55 mL of toluene, 27 mL of ethanol, and 27 mL of distilled water were added to a flask, and then dissolved. Next, it was stirred under reflux at 120° C. for 4 hours. After the reaction was completed, the organic layer was extracted with ethyl acetate and separated by column chromatography to obtain Compound C-94 (4.7 g, yield: 67%).

| Compound | MW | M.P. |
|---|---|---|
| C-94 | 640.2 | 296.3° C. |

### [Example 6] Preparation of Compound C-62

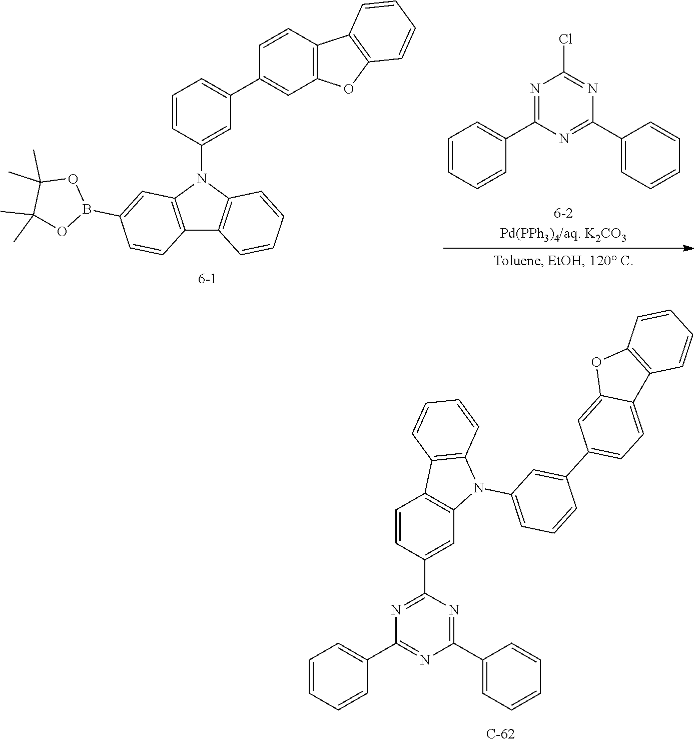

Compound 6-1 (6.0 g, 11.21 mmol), Compound 6-2 (3.9 g, 14.57 mmol), tetrakis(triphenylphosphine)palladium (390 mg, 0.34 mmol), potassium carbonate (3.9 g, 28.01 mmol), 56 mL of toluene, 14 mL of ethanol, and 14 mL of distilled water were added to the reaction vessel, and then stirred at 120° C. for 2 hours. After the reaction was completed, the reaction mixture was cooled to room temperature, the organic layer was extracted with ethyl acetate, and the extracted organic layer was dried over magnesium sulfate, and then the solvent was removed using a rotary evaporator. Then, it was purified by column chromatography to obtain Compound C-62 (4.9 g, yield: 68%).

| Compound | MW | M.P |
|---|---|---|
| C-62 | 640.73 | 294° C. |

### [Example 7] Preparation of Compound C-226-D17

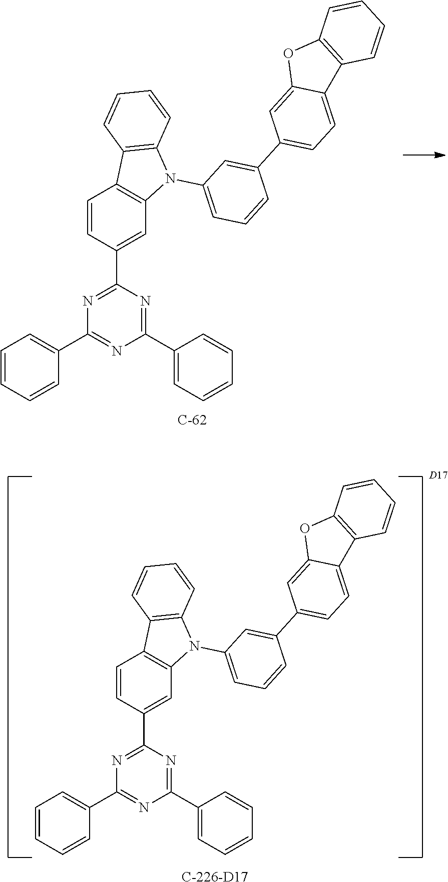

Compound C-62 was synthesized by selecting one among the deuteriumization methods disclosed in Korean Patent Nos. 10-2283849 and 10-1427457, and Compound C-226-D17 (34 g, yield: 77.2%) was obtained.

| Compound | MW | M.P |
|---|---|---|
| C-226-D17 | 657 | 310° C. |

### [Example 8] Preparation of Compound C-226-D10

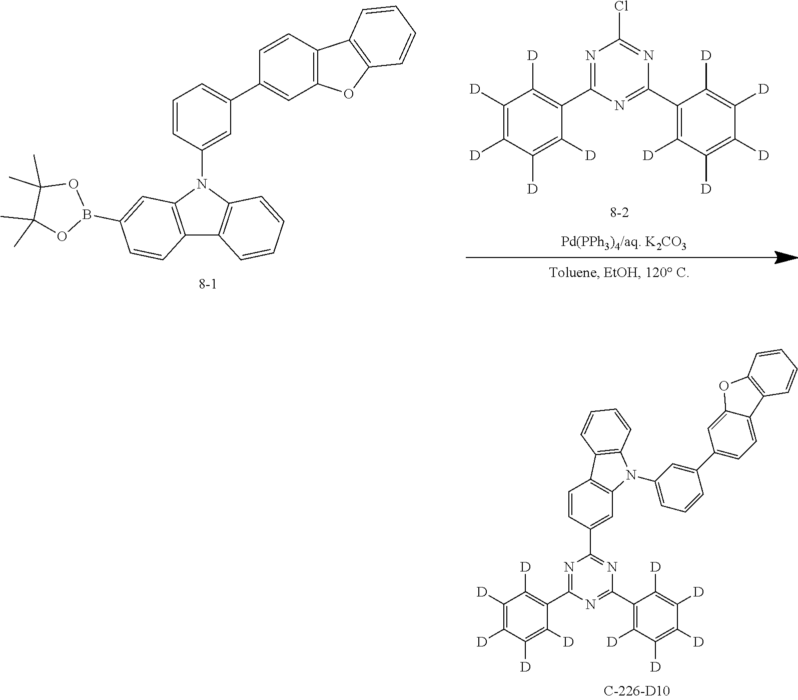 [SMILES: CC1(C)OB(c2ccc3c4ccccc4n(-c4cccc(-c5ccc6c(c5)oc5ccccc56)c4)c3c2)OC1(C)C ; [2H]c1c([2H])c([2H])c(-c2nc(Cl)nc(-c3c([2H])c([2H])c([2H])c([2H])c3[2H])n2)c([2H])c1[2H] ; [2H]c1c([2H])c([2H])c(-c2nc(-c3ccc4c5ccccc5n(-c5cccc(-c6ccc7c(c6)oc6ccccc67)c5)c4c3)nc(-c3c([2H])c([2H])c([2H])c([2H])c3[2H])n2)c([2H])c1[2H]]

Compound 8-1 (7.0 g, 13.07 mmol), Compound 8-2 (4.4 g, 15.69 mmol), tetrakis(triphenylphosphine)palladium (450 mg, 0.39 mmol), potassium carbonate (4.5 g, 32.68 mmol), 65 mL of toluene, 16 mL of ethanol, and 16 mL of distilled water were added to the reaction vessel, and then stirred at 120° C. for 2 hours. After the reaction was completed, the reaction mixture was cooled to room temperature, the organic layer was extracted with ethyl acetate, and the extracted organic layer was dried over magnesium sulfate, and then the solvent was removed using a rotary evaporator. Then, it was purified by column chromatography to obtain Compound C-226-D10 (3.3 g, yield: 39%).

| Compound | MW | M.P |
|---|---|---|
| C-226-D10 | 650.81 | 295° C. |

### [Example 9] Preparation of Compound C-227

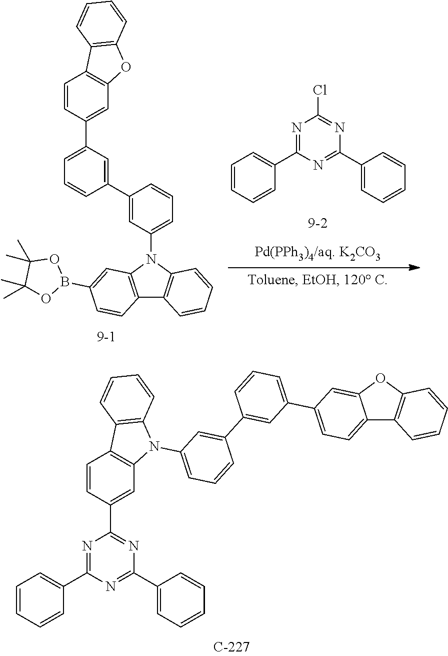 [SMILES: CC1(C)OB(c2ccc3c4ccccc4n(-c4cccc(-c5cccc(-c6ccc7c(c6)oc6ccccc67)c5)c4)c3c2)OC1(C)C ; Clc1nc(-c2ccccc2)nc(-c2ccccc2)n1 ; c1ccc(-c2nc(-c3ccccc3)nc(-c3ccc4c5ccccc5n(-c5cccc(-c6cccc(-c7ccc8c(c7)oc7ccccc78)c6)c5)c4c3)n2)cc1]

Compound 9-1 (9.5 g, 15.53 mmol), Compound 9-2 (5.0 g, 18.64 mmol), tetrakis(triphenylphosphine)palladium (540 mg, 0.47 mmol), potassium carbonate (5.4 g, 38.84 mmol), 77 mL of toluene, 19 mL of ethanol, and 19 mL of distilled water were added to the reaction vessel and then stirred at 120° C. for 2 hours. After the reaction was completed, the reaction mixture was cooled to room temperature, the organic layer was extracted with ethyl acetate, and the extracted organic layer was dried over magnesium sulfate, and then the solvent was removed using a rotary evaporator. Then, it was purified by column chromatography to obtain Compound C-227 (8.5 g, yield: 76%).

| Compound | MW | M.P |
|---|---|---|
| C-227 | 716.84 | 270° C. |

### [Example 10] Preparation of Compound C-228

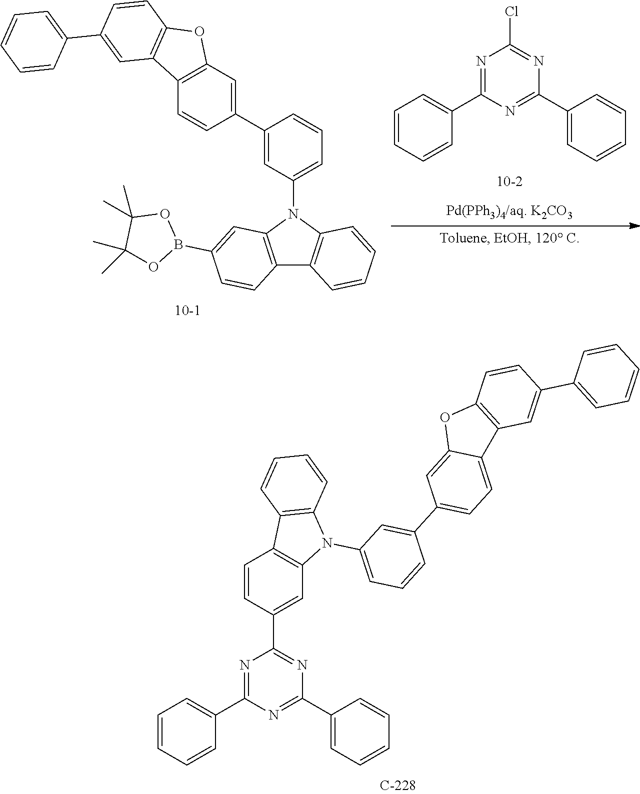 [SMILES: CC1(C)OB(c2ccc3c4ccccc4n(-c4cccc(-c5ccc6c(c5)oc5ccc(-c7ccccc7)cc56)c4)c3c2)OC1(C)C ; Clc1nc(-c2ccccc2)nc(-c2ccccc2)n1 ; c1ccc(-c2ccc3oc4cc(-c5cccc(-n6c7ccccc7c7ccc(-c8nc(-c9ccccc9)nc(-c9ccccc9)n8)cc76)c5)ccc4c3c2)cc1]

Compound 10-1 (7.0 g, 11.45 mmol), Compound 10-2 (3.7 g, 13.74 mmol), tetrakis(triphenylphosphine)palladium (400 mg, 0.34 mmol), potassium carbonate (4.0 g, 28.62 mmol), 57 mL of toluene, 14 mL of ethanol, and 14 mL of distilled water were added to the reaction vessel and then stirred at 120° C. for 2 hours. After the reaction was completed, the reaction mixture was cooled to room temperature, the organic layer was extracted with ethyl acetate, and the extracted organic layer was dried over magnesium sulfate, and then the solvent was removed using a rotary evaporator. Then, it was purified by column chromatography to obtain Compound C-228 (3.0 g, yield: 37%).

| Compound | MW | M.P |
|---|---|---|
| C-228 | 716.84 | 285° C. |

### [Example 11] Preparation of Compound C-229

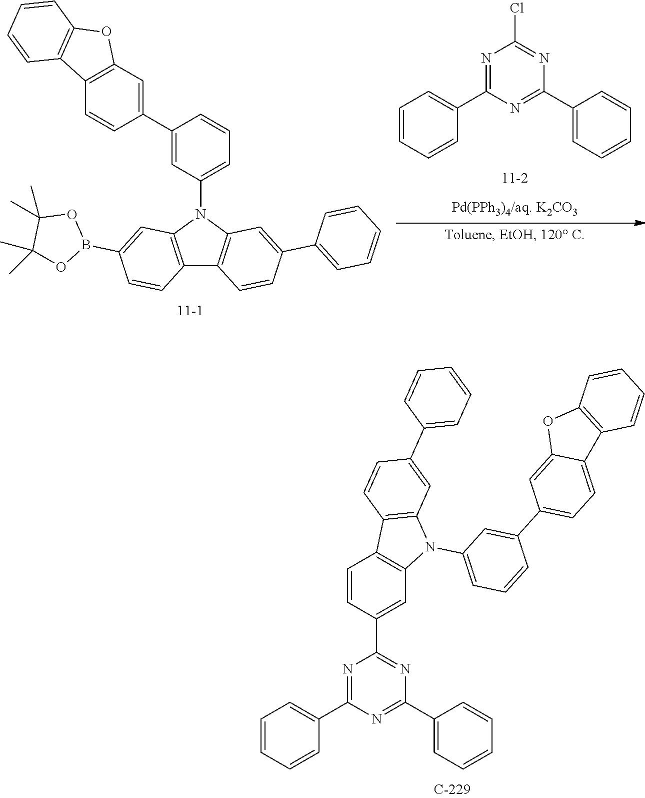 [SMILES: CC1(C)OB(c2ccc3c4ccc(-c5ccccc5)cc4n(-c4cccc(-c5ccc6c(c5)oc5ccccc56)c4)c3c2)OC1(C)C ; Clc1nc(-c2ccccc2)nc(-c2ccccc2)n1 ; c1ccc(-c2ccc3c4ccc(-c5nc(-c6ccccc6)nc(-c6ccccc6)n5)cc4n(-c4cccc(-c5ccc6c(c5)oc5ccccc56)c4)c3c2)cc1]

Compound 11-1 (4.3 g, 7.03 mmol), Compound 11-2 (2.3 g, 8.44 mmol), tetrakis(triphenylphosphine)palladium (240 mg, 0.21 mmol), potassium carbonate (2.4 g, 17.58 mmol), 35 mL of toluene, 9 mL of ethanol, and 9 mL of distilled water were added to the reaction vessel and then stirred at 120° C. for 2 hours. After the reaction was completed, the reaction mixture was cooled to room temperature, the organic layer was extracted with ethyl acetate, and the extracted organic layer was dried over magnesium sulfate, and then the solvent was removed using a rotary evaporator. Then, it was purified by column chromatography to obtain Compound C-229 (2.0 g, yield: 40%).

| Compound | MW | M.P |
|---|---|---|
| C-229 | 716.84 | 314° C. |

### [Example 12] Preparation of Compound C-230

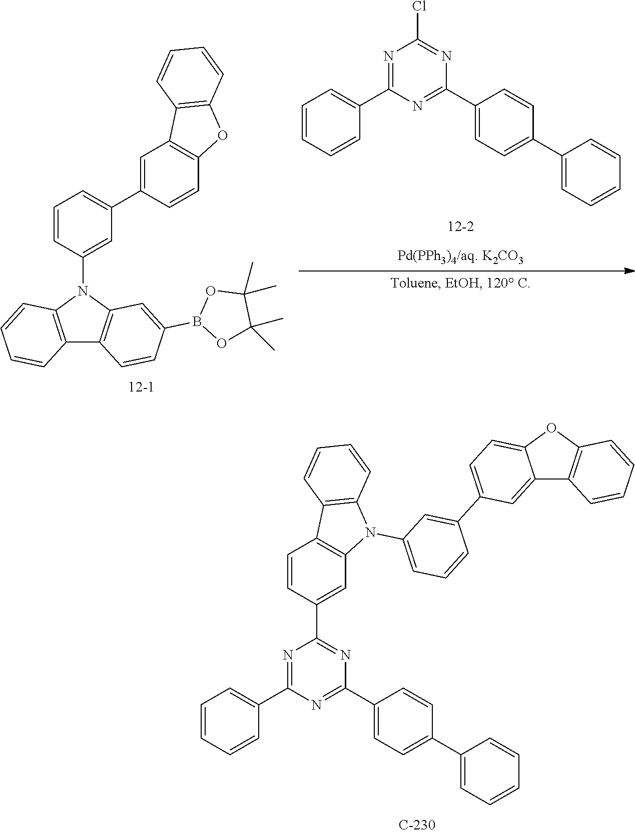 [SMILES: CC1(C)OB(c2ccc3c4ccccc4n(-c4cccc(-c5ccc6oc7ccccc7c6c5)c4)c3c2)OC1(C)C ; Clc1nc(-c2ccccc2)nc(-c2ccc(-c3ccccc3)cc2)n1 ; c1ccc(-c2ccc(-c3nc(-c4ccccc4)nc(-c4ccc5c6ccccc6n(-c6cccc(-c7ccc8oc9ccccc9c8c7)c6)c5c4)n3)cc2)cc1]

Compound 12-1 (3.0 g, 5.60 mmol), Compound 12-2 (2.5 g, 7.28 mmol), tetrakis(triphenylphosphine)palladium (190 mg, 0.17 mmol), potassium carbonate (1.9 g, 14.01 mmol), 28 mL of toluene, 7 mL of ethanol, and 7 mL of distilled water were added to the reaction vessel and then stirred at 120° C. for 2 hours. After the reaction was completed, the reaction mixture was cooled to room temperature, the organic layer was extracted with ethyl acetate, and the extracted organic layer was dried over magnesium sulfate, and then the solvent was removed using a rotary evaporator. Then, it was purified by column chromatography to obtain Compound C-230 (2.7 g, yield: 67%).

| Compound | MW | M.P |
|---|---|---|
| C-230 | 716.84 | 253° C. |

### [Example 13] Preparation of Compound C-231

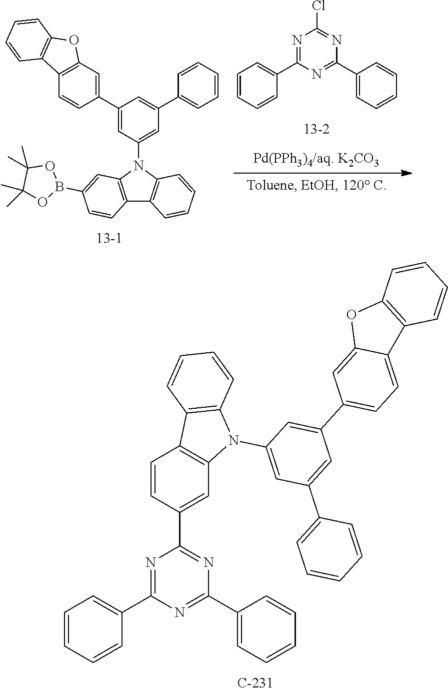

Compound 13-1 (4.3 g, 7.03 mmol), Compound 13-2 (2.3 g, 8.44 mmol), tetrakis(triphenylphosphine)palladium (240 mg, 0.21 mmol), potassium carbonate (2.4 g, 17.58 mmol), 35 mL of toluene, 9 mL of ethanol, and 9 mL of distilled water were added to the reaction vessel and then stirred at 120° C. for 2 hours. After the reaction was completed, the reaction mixture was cooled to room temperature, the organic layer was extracted with ethyl acetate, and the extracted organic layer was dried over magnesium sulfate, and then the solvent was removed using a rotary evaporator. Then, it was purified by column chromatography to obtain Compound C-231 (2.0 g, yield: 40%).

| Compound | MW | M.P |
|---|---|---|
| C-231 | 716.84 | 245° C. |

### [Example 14] Preparation of Compound C-91

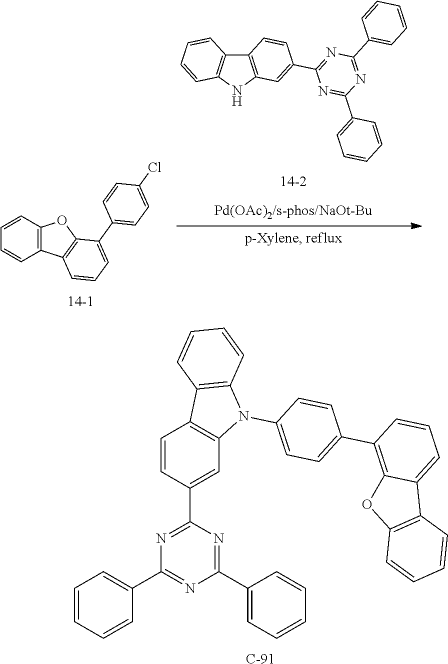

Compound 14-1 (3.9 g, 13.80 mmol), Compound 14-2 (5.0 g, 12.66 mmol), palladium acetate (140 mg, 0.63 mmol), 2-dicyclohexylphosphino-2′,6′-dimethoxybiphenyl (520 mg, 1.25 mmol), sodium tert-butoxide (1.8 g, 18.82 mmol), and 62 mL of o-xylene were added to the reaction vessel and then stirred under reflux for 2 hours. After the reaction was completed, the reaction mixture was cooled to room temperature, the organic layer was extracted with ethyl acetate, and the extracted organic layer was dried over magnesium sulfate, and then the solvent was removed using a rotary evaporator. Then, it was purified by column chromatography to obtain Compound C-91 (6.4 g, yield: 80%).

| Compound | MW | M.P |
|---|---|---|
| C-91 | 640.75 | 345° C. |

### [Example 15] Preparation of Compound C-232

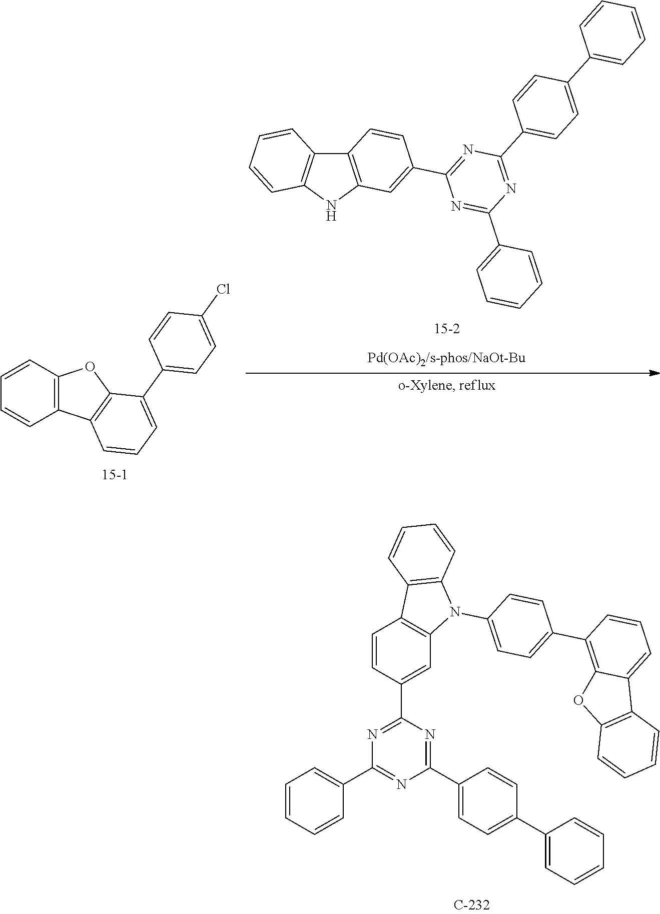 [SMILES: Clc1ccc(-c2cccc3c2oc2ccccc23)cc1 ; c1ccc(-c2ccc(-c3nc(-c4ccccc4)nc(-c4ccc5c(c4)[nH]c4ccccc45)n3)cc2)cc1 ; c1ccc(-c2ccc(-c3nc(-c4ccccc4)nc(-c4ccc5c6ccccc6n(-c6ccc(-c7cccc8c7oc7ccccc78)cc6)c5c4)n3)cc2)cc1]

Compound 15-1 (3.5 g, 12.64 mmol), Compound 15-2 (5.0 g, 10.54 mmol), palladium acetate (240 mg, 1.05 mmol), 2-dicyclohexylphosphino-2′,6′-dimethoxybiphenyl (870 mg, 2.11 mmol), sodium tert-butoxide (2.0 g, 21.07 mmol), and 53 mL of o-xylene were added to the reaction vessel and then stirred for reflux for 2 hours. After the reaction was completed, the reaction mixture was cooled to room temperature, the organic layer was extracted with ethyl acetate, and the extracted organic layer was dried over magnesium sulfate, and then the solvent was removed using a rotary evaporator. Then, it was purified by column chromatography to obtain Compound C-232 (6.4 g, yield: 80%).

| Compound | MW | M.P |
|---|---|---|
| C-232 | 716.84 | 311° C. |

### [Example 16] Preparation of Compound C-123

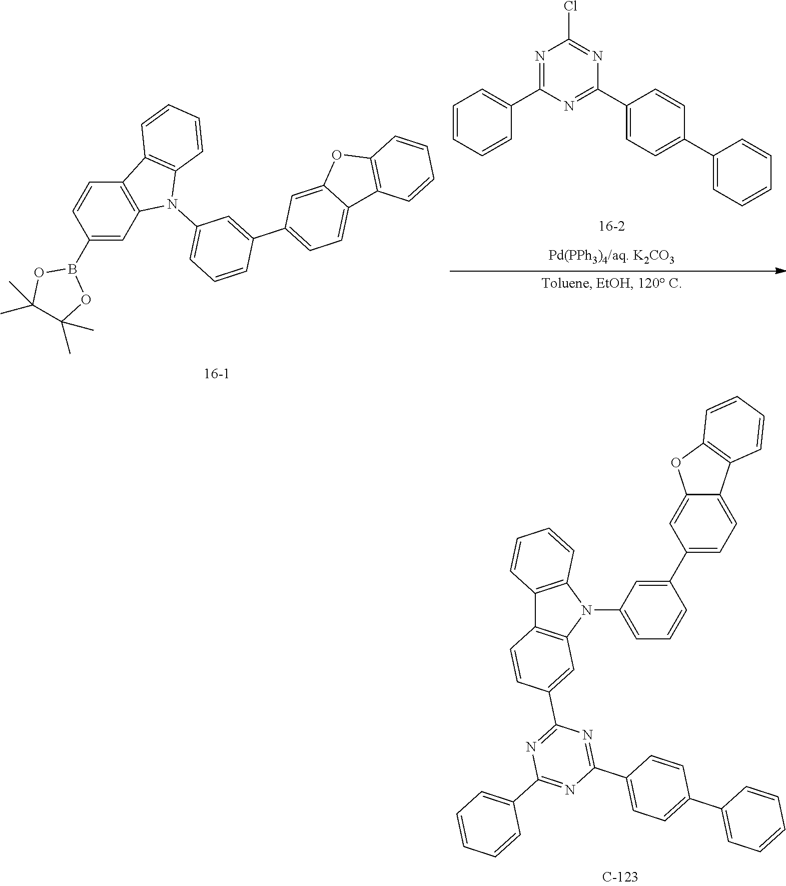

Compound 16-1 (5 g, 9.34 mmol), Compound 16-2 (3.5 g, 10.27 mmol), tetrakis(triphenylphosphine)palladium (320 mg, 0.28 mmol), potassium carbonate (3.2 g, 23.34 mmol), 47 mL of toluene, 12 mL of ethanol, and 12 mL of distilled water were added to the reaction vessel and then stirred at 120° C. for 2 hours. After the reaction was completed, the reaction mixture was cooled to room temperature, the organic layer was extracted with ethyl acetate, and the extracted organic layer was dried over magnesium sulfate, and then the solvent was removed using a rotary evaporator. Then, it was purified by column chromatography to obtain Compound C-123 (3.5 g, yield: 52%).

| Compound | MW | M.P |
|---|---|---|
| C-123 | 716.84 | 299° C. |

### [Example 17] Preparation of Compound C-122

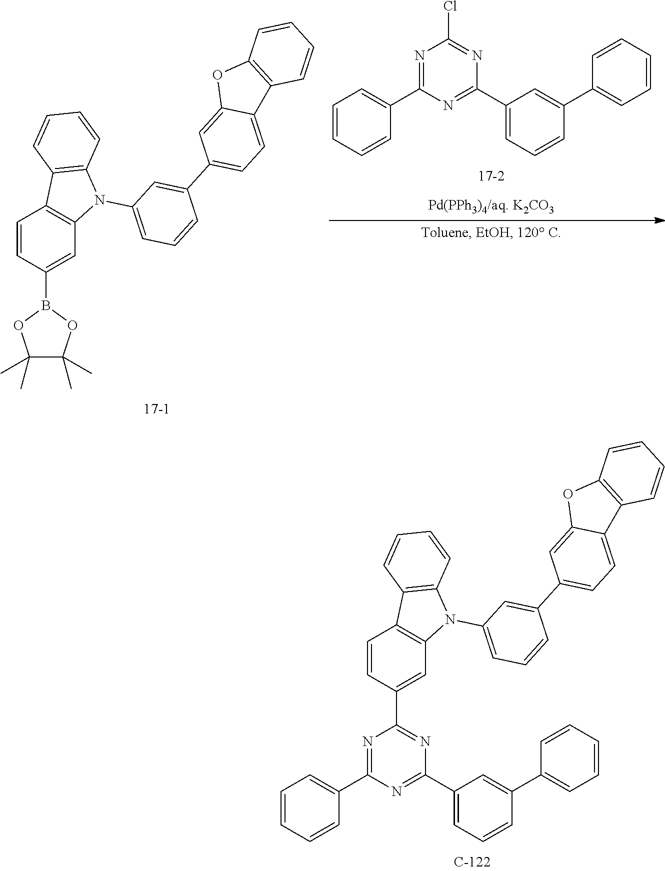

Compound 17-1 (5.0 g, 9.34 mmol), Compound 17-2 (3.85 g, 11.2 mmol), tetrakis(triphenylphosphine)palladium (0.32 g, 0.28 mmol), potassium carbonate (3.2 g, 23.35 mmol), 46 mL of toluene, 12 mL of ethanol, and 12 mL of distilled water were added to a flask, and then dissolved. Next, it was stirred under reflux at 4 hours. After the reaction was completed, the organic layer was extracted with ethyl acetate and separated by column chromatography to obtain Compound C-122 (3.4 g, yield: 51%).

| Compound | MW | M.P |
|---|---|---|
| C-122 | 716.8 | 233° C. |

### [Example 18] Preparation of Compound C-236

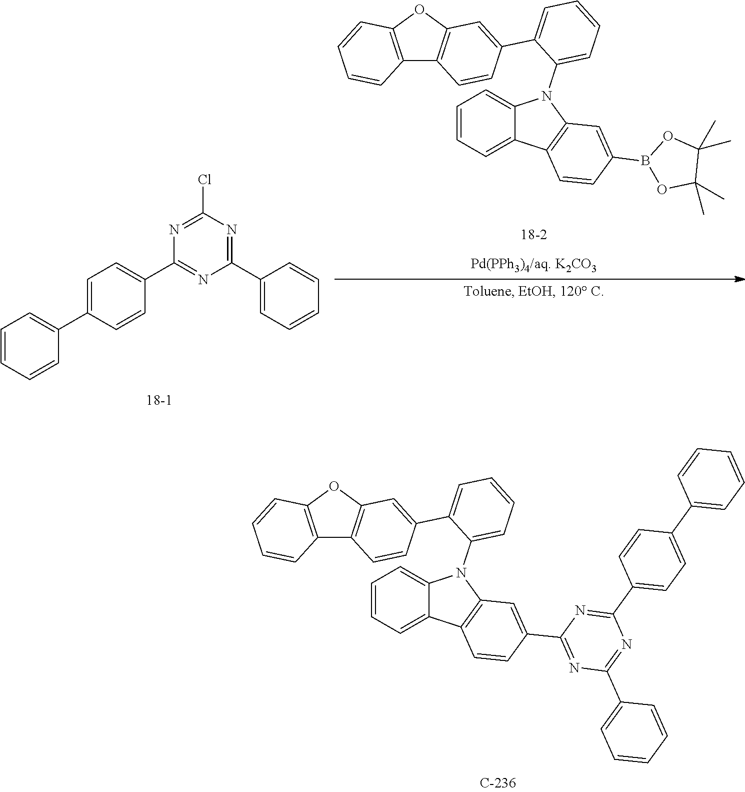 [SMILES: Clc1nc(-c2ccccc2)nc(-c2ccc(-c3ccccc3)cc2)n1 ; CC1(C)OB(c2ccc3c4ccccc4n(-c4ccccc4-c4ccc5c(c4)oc4ccccc45)c3c2)OC1(C)C ; c1ccc(-c2ccc(-c3nc(-c4ccccc4)nc(-c4ccc5c6ccccc6n(-c6ccccc6-c6ccc7c(c6)oc6ccccc67)c5c4)n3)cc2)cc1]

Compound 18-1 (4.5 g, 13.07 mmol), Compound 18-2 (7 g, 13.07 mmol), tetrakis(triphenylphosphine)palladium (750 mg, 0.653 mmol), potassium carbonate (5.4 g, 39.22 mmol), 80 mL of toluene, 20 mL of ethanol, and 20 mL of distilled water were added to the reaction vessel, and then stirred under reflux at 140′C for 2 hours. After the reaction was completed, the reaction mixture was cooled to room temperature, the organic layer was extracted with ethyl acetate, and the extracted organic layer was dried over magnesium sulfate, and then the solvent was removed using a rotary evaporator. Then, it was purified by column chromatography to obtain Compound C-236 (1.4 g, yield: 14%).

| Compound | MW | M.P |
|---|---|---|
| C-236 | 716.8 | 244.5° C. |

### [Example 19] Preparation of Compound C-233

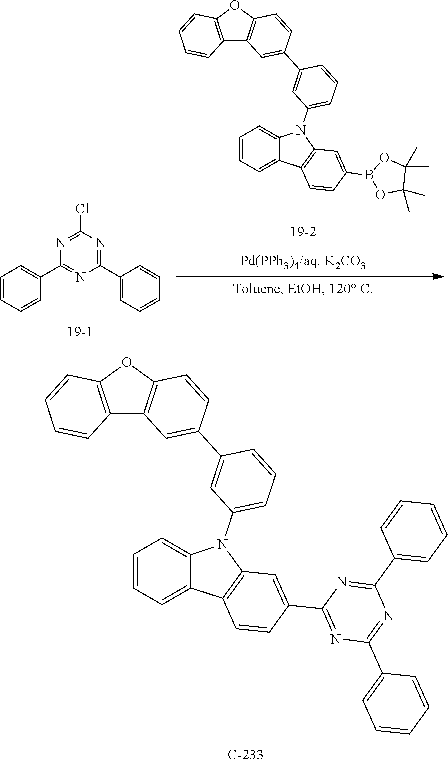 [SMILES: Clc1nc(-c2ccccc2)nc(-c2ccccc2)n1 ; CC1(C)OB(c2ccc3c4ccccc4n(-c4cccc(-c5ccc6oc7ccccc7c6c5)c4)c3c2)OC1(C)C ; c1ccc(-c2nc(-c3ccccc3)nc(-c3ccc4c5ccccc5n(-c5cccc(-c6ccc7oc8ccccc8c7c6)c5)c4c3)n2)cc1]

Compound 19-1 (1.9 g, 7.097 mmol), Compound 19-2 (3.8 g, 7.097 mmol), tetrakis(triphenylphosphine)palladium (410 mg, 0.354 mmol), potassium carbonate (3 g, 21.29 mmol), 40 mL of toluene, 10 mL of ethanol, and 10 mL of distilled water were added to the reaction vessel and then stirred under reflux at 140° C. for 2 hours. After the reaction was completed, the reaction mixture was cooled to room temperature, the organic layer was extracted with ethyl acetate, and the extracted organic layer was dried over magnesium sulfate, and then the solvent was removed using a rotary evaporator. Then, it was purified by column chromatography to obtain Compound C-233 (3 g, yield: 66%).

| Compound | MW | M.P |
|---|---|---|
| C-233 | 640.7 | 255.8° C. |

### [Example 20] Preparation of Compound C-93

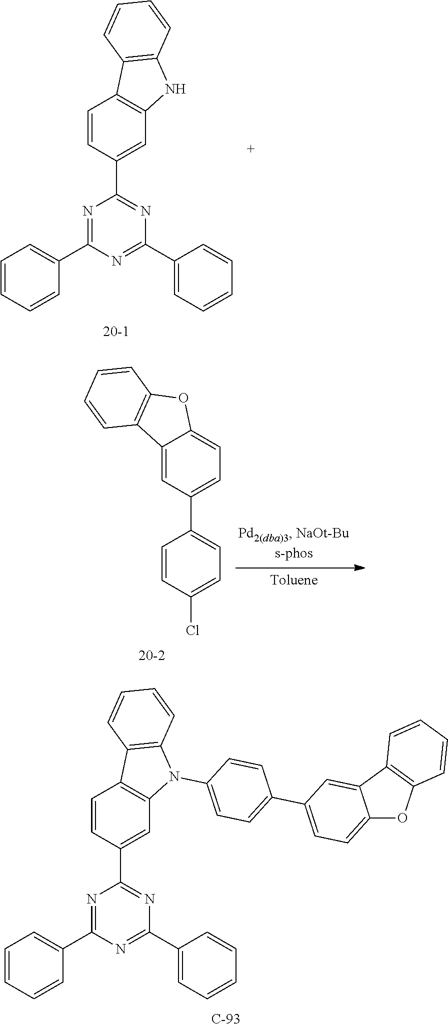

Compound 20-1 (5 g, 12.55 mmol), Compound 20-2 (3.8 g, 13.80 mmol), tris(dibenzylideneacetone)dipalladium(0) (0.57 g, 0.627 mmol), sodium tert-butoxide (1.8 g, 18.82 mmol), and 2-dicyclohexylphosphino-2′,6′-dimethoxybiphenyl (0.51 g, 1.255 mmol) were added to 60 mL of toluene, and then stirred under reflux at 120° C. for 1 hour. After completion of the reaction, the mixture was cooled to room temperature and filtered through Celite. After distillation under reduced pressure, it was separated by column chromatography to obtain Compound C-93 (2 g, yield: 24%).

| Compound | MW | M.P |
|---|---|---|
| C-93 | 640.7 | 277.3° C. |

### [Example 21] Preparation of Compound C-92

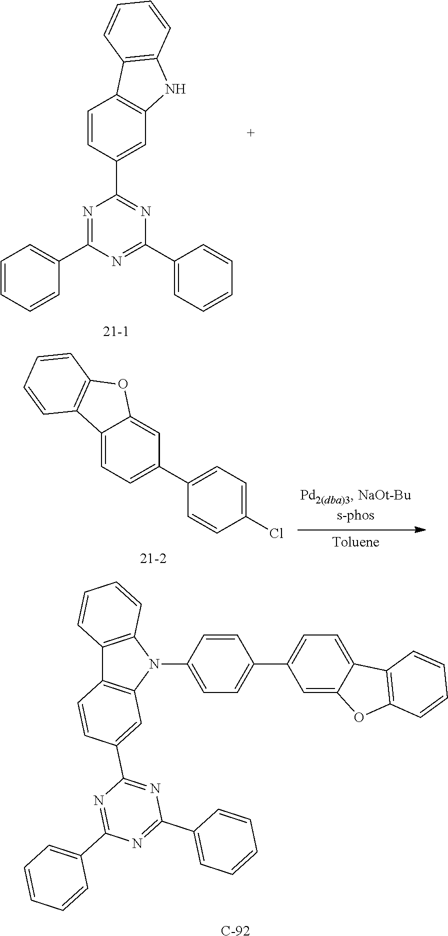

Compound 21-1 (5 g, 12.55 mmol), Compound 21-2 (3.8 g, 13.80 mmol), tris(dibenzylideneacetone)dipalladium(0) (0.57 g, 0.627 mmol), sodium tert-butoxide (1.8 g, 18.82 mmol), and 2-dicyclohexylphosphino-2′,6′-dimethoxybiphenyl (0.51 g, 1.255 mmol) were added to 60 mL of toluene, and then stirred under reflux at 120° C. for 1 hour. After completion of the reaction, the reaction mixture was cooled to room temperature and filtered through Celite. After distillation under reduced pressure, it was separated by column chromatography to obtain Compound C-92 (4.4 g, yield: 54%).

| Compound | MW | M.P |
|---|---|---|
| C-92 | 640.7 | 309.9° C. |

### [Example 22] Preparation of Compound C-234

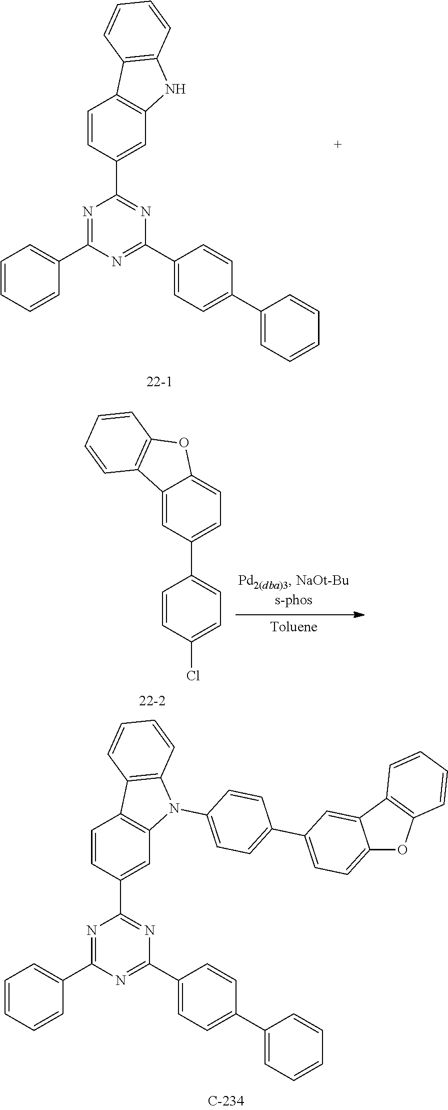 [SMILES: c1ccc(-c2ccc(-c3nc(-c4ccccc4)nc(-c4ccc5c(c4)[nH]c4ccccc45)n3)cc2)cc1 ; Clc1ccc(-c2ccc3oc4ccccc4c3c2)cc1 ; c1ccc(-c2ccc(-c3nc(-c4ccccc4)nc(-c4ccc5c6ccccc6n(-c6ccc(-c7ccc8oc9ccccc9c8c7)cc6)c5c4)n3)cc2)cc1]

Compound 22-1 (5 g, 10.53 mmol), Compound 22-2 (3.2 g, 11.59 mmol), tris(dibenzylideneacetone)dipalladium(0) (0.48 g, 0.526 mmol), sodium tert-butoxide (1.5 g, 15.80 mmol), 2-dicyclohexylphosphino-2′,6′-dimethoxybiphenyl (0.43 g, 1.053 mmol) were added to 55 mL of toluene, and then stirred under reflux at 120° C. for 1 hour. After completion of the reaction, the reaction mixture was cooled to room temperature and filtered through Celite. After distillation under reduced pressure, it was separated by column chromatography to obtain Compound C-234 (4.5 g, yield: 59%).

| Compound | MW | M.P |
|---|---|---|
| C-234 | 716.8 | 290° C. |

### [Example 23] Preparation of Compound C-235

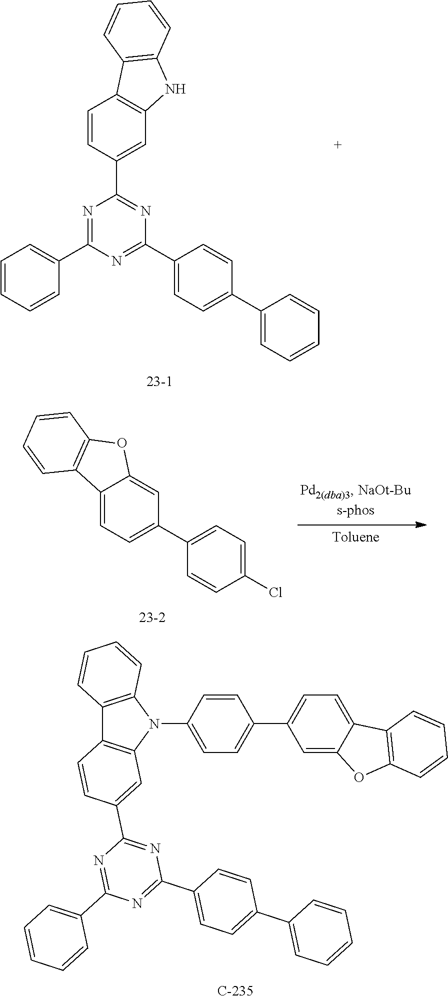 [SMILES: c1ccc(-c2ccc(-c3nc(-c4ccccc4)nc(-c4ccc5c(c4)[nH]c4ccccc45)n3)cc2)cc1 ; Clc1ccc(-c2ccc3c(c2)oc2ccccc23)cc1 ; c1ccc(-c2ccc(-c3nc(-c4ccccc4)nc(-c4ccc5c6ccccc6n(-c6ccc(-c7ccc8c(c7)oc7ccccc78)cc6)c5c4)n3)cc2)cc1]

Compound 23-1 (5 g, 10.53 mmol), Compound 23-2 (3.2 g, 11.59 mmol), tris(dibenzylideneacetone)dipalladium(0) (0.48 g, 0.526 mmol), sodium tert-butoxide (1.5 g, 15.80 mmol), 2-dicyclohexylphosphino-2′,6′-dimethoxybiphenyl (0.43 g, 1.053 mmol) were added to 55 mL of toluene, and then stirred under reflux at 120° C. for 1 hour. After completion of the reaction, the reaction mixture was cooled to room temperature and filtered through Celite. After distillation under reduced pressure, it was separated by column chromatography to obtain Compound C-235 (4.3 g, yield: 56%).

| Compound | MW | M.P |
|---|---|---|
| C-235 | 716.8 | 300° C. |

### [Device Examples 1 to 12] Preparation of Green OLEDs Deposited with a Plurality of Host Materials According to the Present Disclosure

OLEDs according to the present disclosure were produced. First, a transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and thereafter was stored in isopropyl alcohol and then used. Thereafter, the ITO substrate was mounted on a substrate holder of a vacuum vapor deposition apparatus. Then, Compound HI-1 was introduced into a cell of the vacuum vapor deposition apparatus, and Compound HT-1 was introduced into another cell. The two materials were evaporated at different rates and Compound HI-1 was deposited in a doping amount of 3 wt % based on the total amount of the compounds HI-1 and HT-1 to form a hole injection layer having a thickness of 10 nm. Next, Compound HT-1 was deposited as a first hole transport layer having a thickness of 80 nm on the hole injection layer. Compound HT-2 was then introduced into another cell of the vacuum vapor deposition apparatus and was evaporated by applying an electric current to the cell, thereby forming a second hole transport layer having a thickness of 30 nm on the first hole transport layer. After forming the hole injection layer and the hole transport layers, a light-emitting layer was formed thereon as follows: The first host compound and the second host compound described in the following Table 1 were introduced into two cells of the vacuum vapor deposition apparatus as hosts, respectively, and Compound D-130 was introduced into another cell as a dopant. The two host materials were evaporated at a rate of 1:2 (the first host:the second host), and the dopant material was evaporated at a different rate, simultaneously and deposited in a doping amount of 10 wt % based on the total amount of the hosts and dopant to form a light-emitting layer having a thickness of 40 nm on the second hole transport layer. Next, compounds ETL-1 and EIL-1 as electron transport materials were deposited at a weight ratio of 40:60 to form an electron transport layer having a thickness of 35 nm on the light-emitting layer. After depositing Compound EIL-1 as an electron injection layer having a thickness of 2 nm on the electron transport layer, an Al cathode having a thickness of 80 nm was deposited on the electron injection layer by another vacuum vapor deposition apparatus. Thus, OLEDs were produced. Each compound used for all the materials were purified by vacuum sublimation under 10⁻⁶ torr.

### [Comparative Example 1] Preparation of an OLED Including Comparative Compound as Hosts

An OLED was manufactured in the same manner as in Device Example 1, except that each of the first host compound and the second host compound described in the following Table 1 was used as the hosts of the light-emitting layer.

The driving voltage, luminous efficiency, and the luminous color at a luminance of 1,000 nits and the time taken for luminance to decrease from 100% to 80% at a luminance of 60,000 nits (lifespan: T80) of the OLEDs of Device Examples 1 to 12 and Comparative Example 1 produced as described above, are measured, and then the results thereof are shown in the following Table 1.

TABLE 1

| | | | Driving | Luminous | | |
|---|---|---|---|---|---|---|
| | | | Voltage | Efficiency | Luminous | Lifespan |
| | First Host | Second Host | [V] | [cd/A] | Color | (T80) [hr] |
| Device Example 1 | 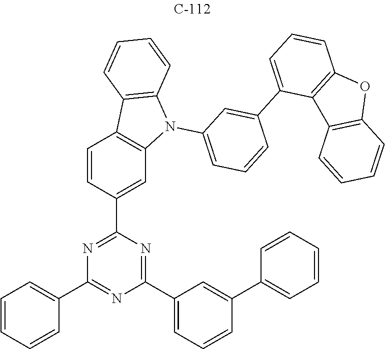 [SMILES: c1ccc(-c2cccc(-c3nc(-c4ccccc4)nc(-c4ccc5c6ccccc6n(-c6cccc(-c7cccc8oc9ccccc9c78)c6)c5c4)n3)c2)cc1] | 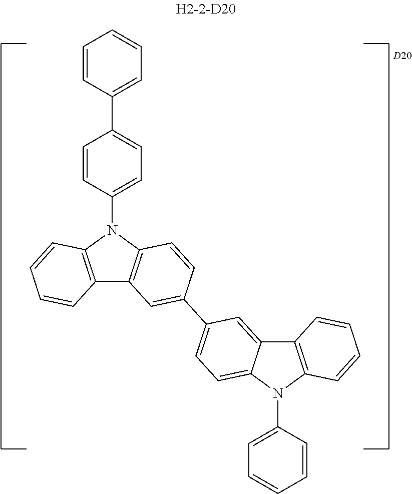 | 3.0 | 108.6 | Green | 135.0 |
| Device Example 2 | 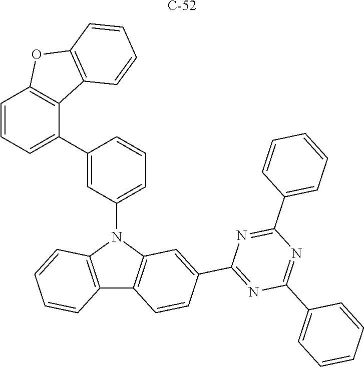 | 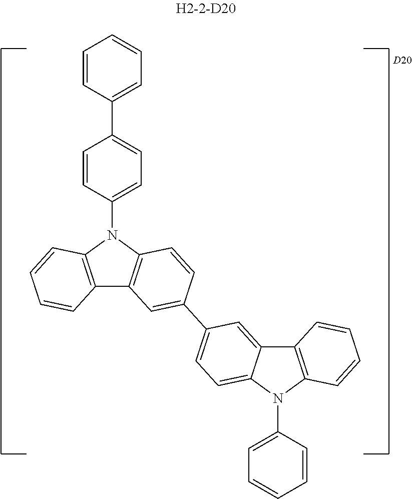 | 3.0 | 108.5 | Green | 124.0 |
| Device Example 3 | 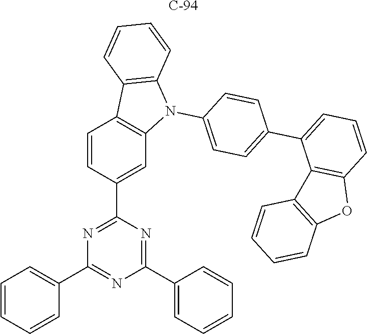 | 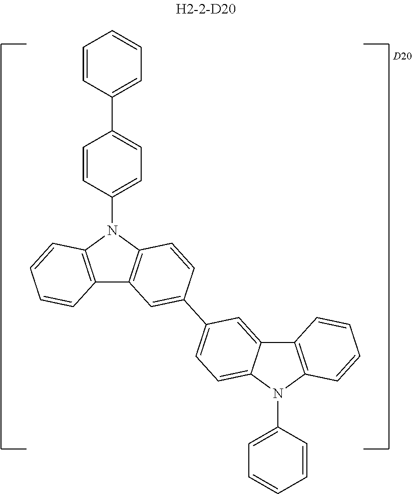 | 2.9 | 108.4 | Green | 114.6 |
| Device Example 4 | 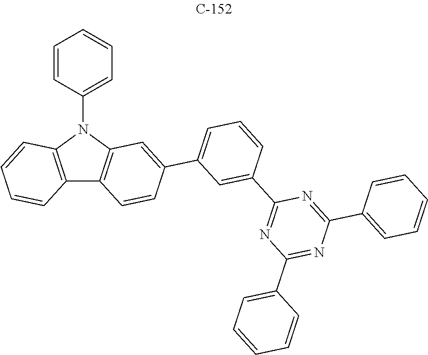 [SMILES: c1ccc(-c2nc(-c3ccccc3)nc(-c3cccc(-c4ccc5c6ccccc6n(-c6ccccc6)c5c4)c3)n2)cc1] | 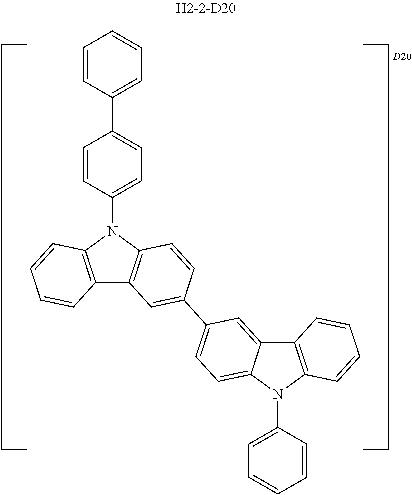 | 3.0 | 108.2 | Green | 87.6 |
| Device Example 5 | 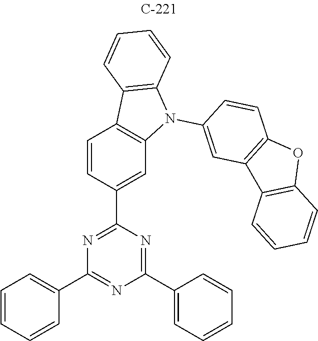 [SMILES: c1ccc(-c2nc(-c3ccccc3)nc(-c3ccc4c5ccccc5n(-c5ccc6oc7ccccc7c6c5)c4c3)n2)cc1] | 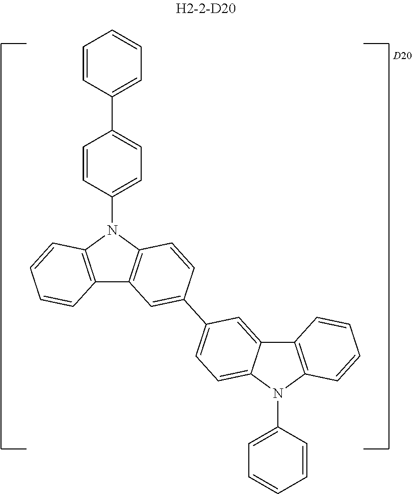 | 2.9 | 108.2 | Green | 82.6 |
| Device Example 6 | 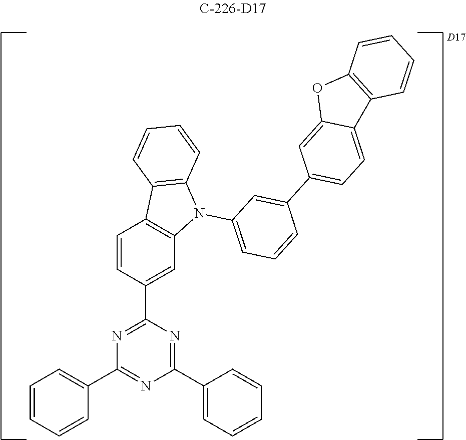 | 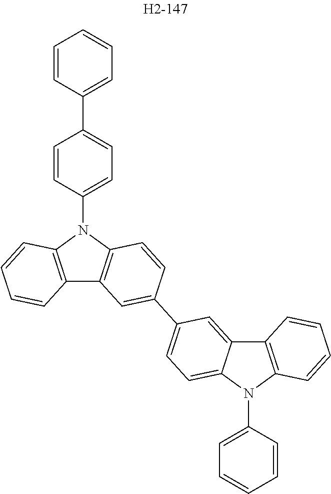 [SMILES: c1ccc(-c2ccc(-n3c4ccccc4c4cc(-c5ccc6c(c5)c5ccccc5n6-c5ccccc5)ccc43)cc2)cc1] | 3.0 | 106.4 | Green | 120.1 |
| Device Example 7 | 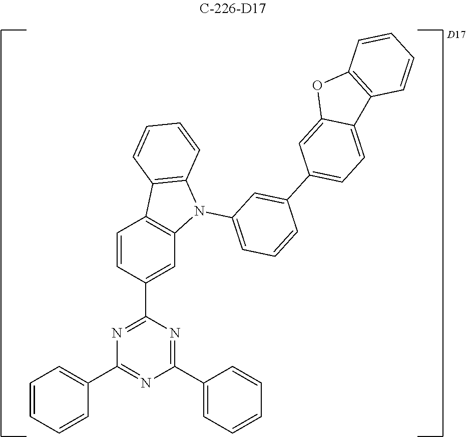 | 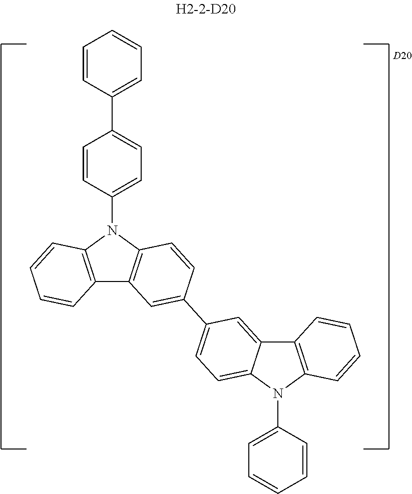 | 3.0 | 106.2 | Green | 178.3 |
| Device Example 8 | 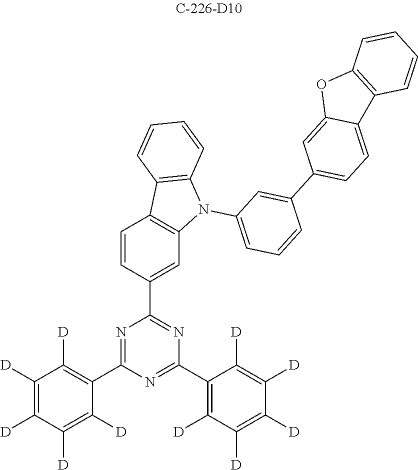 [SMILES: [2H]c1c([2H])c([2H])c(-c2nc(-c3ccc4c5ccccc5n(-c5cccc(-c6ccc7c(c6)oc6ccccc67)c5)c4c3)nc(-c3c([2H])c([2H])c([2H])c([2H])c3[2H])n2)c([2H])c1[2H]] | 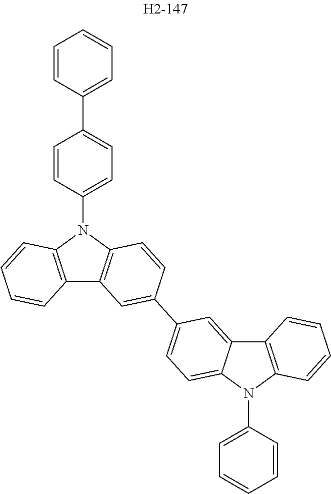 [SMILES: c1ccc(-c2ccc(-n3c4ccccc4c4cc(-c5ccc6c(c5)c5ccccc5n6-c5ccccc5)ccc43)cc2)cc1] | 3.0 | 106.8 | Green | 117.5 |
| Device Example 9 | 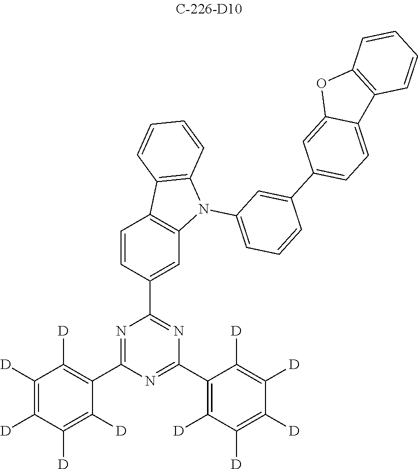 [SMILES: [2H]c1c([2H])c([2H])c(-c2nc(-c3ccc4c5ccccc5n(-c5cccc(-c6ccc7c(c6)oc6ccccc67)c5)c4c3)nc(-c3c([2H])c([2H])c([2H])c([2H])c3[2H])n2)c([2H])c1[2H]] | 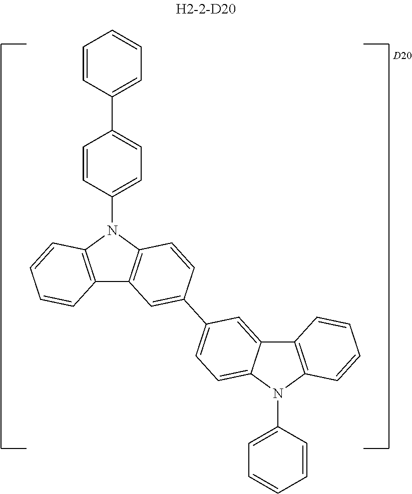 | 3.0 | 107.1 | Green | 156.7 |
| Device Example 10 | 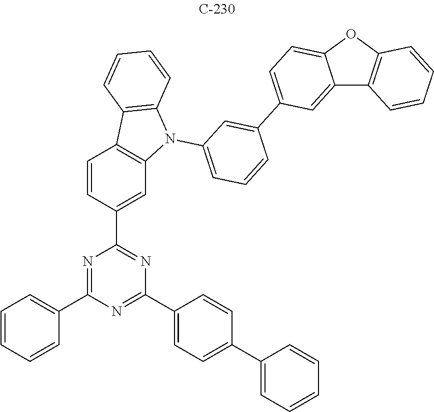 [SMILES: c1ccc(-c2ccc(-c3nc(-c4ccccc4)nc(-c4ccc5c6ccccc6n(-c6cccc(-c7ccc8oc9ccccc9c8c7)c6)c5c4)n3)cc2)cc1] | 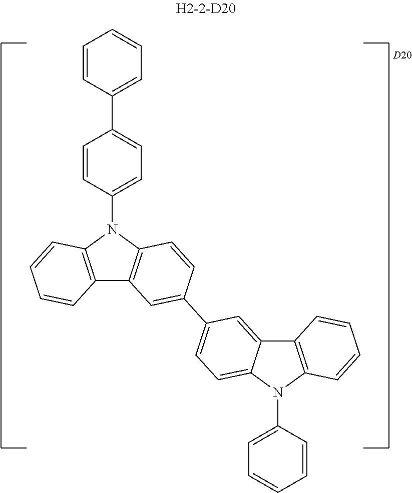 | 2.8 | 103.4 | Green | 89.2 |
| Device Example 11 | 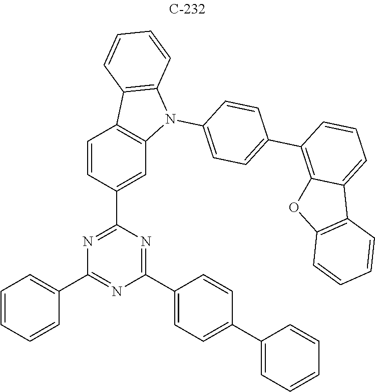 [SMILES: c1ccc(-c2ccc(-c3nc(-c4ccccc4)nc(-c4ccc5c6ccccc6n(-c6ccc(-c7cccc8c7oc7ccccc78)cc6)c5c4)n3)cc2)cc1] | 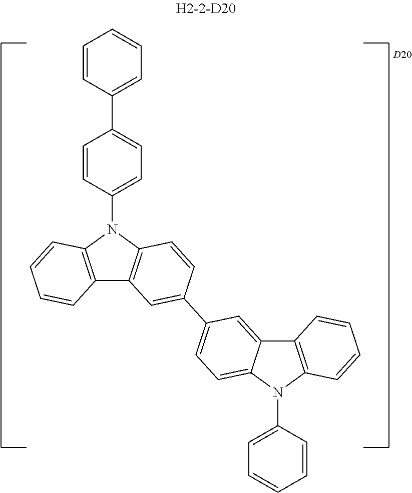 | 3.2 | 98.3 | Green | 139.3 |
| Device Example 12 | 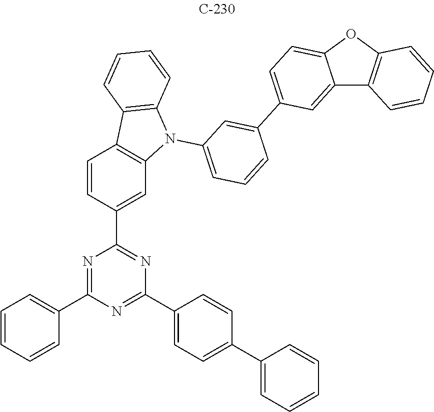 [SMILES: c1ccc(-c2ccc(-c3nc(-c4ccccc4)nc(-c4ccc5c6ccccc6n(-c6cccc(-c7ccc8oc9ccccc9c8c7)c6)c5c4)n3)cc2)cc1] | 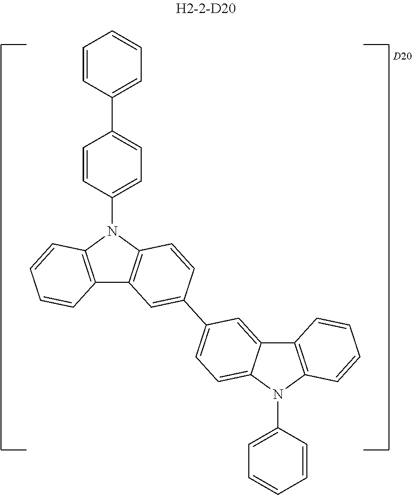 | 2.9 | 101.4 | Green | 107.2 |
| Comparative Example 1 | 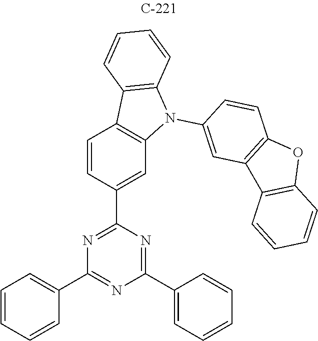 [SMILES: c1ccc(-c2nc(-c3ccccc3)nc(-c3ccc4c5ccccc5n(-c5ccc6oc7ccccc7c6c5)c4c3)n2)cc1] | 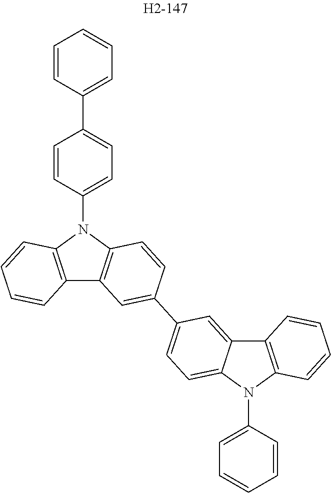 [SMILES: c1ccc(-c2ccc(-n3c4ccccc4c4cc(-c5ccc6c(c5)c5ccccc5n6-c5ccccc5)ccc43)cc2)cc1] | 2.9 | 108.2 | Green | 70.4 |

As shown in Table 1 above, it was confirmed that the organic electroluminescent devices using a plurality of host materials according to the present disclosure (Device Examples 1 to 12) show excellent lifespan properties compared to the organic electroluminescent device comprising a conventional host combination (Comparative Example 1).

Green light-emitting organic electroluminescent devices generally have a shorter lifespan than red light-emitting organic electroluminescent devices. In order to improve the lifespan properties of a green light-emitting organic electroluminescent device, a compound introduced with a deuterated moiety was used in the present disclosure. Although not limited by theory, when an organic electroluminescent compound is substituted with deuterium, the compound's stability can be increased by lowering the zero point vibration energy of the compound and increasing the bond dissociation energy (BDE) in the compound.

### [Device Examples 13 to 17] Preparation of Green OLEDs Deposited with an Organic Electroluminescent Compound According to the Present Disclosure

OLEDs were manufactured in the same manner as in Device Example 1, except that the light-emitting layer was deposited as follows.

The host compound described in the following Table 2 was introduced into a cell of the vacuum vapor deposition apparatus as a host and Compound D-130 was introduced into another cell as a dopant. The host material and the dopant material were evaporated at different rates, simultaneously and the dopant material was deposited in a doping amount of 10 wt % based on the total amount of the host and dopant to form a light-emitting layer having a thickness of 40 nm on the second hole transport layer.

### [Comparative Example 2] Preparation of an OLED Including Comparative Compound as a Host

An OLED was manufactured in the same manner as in Device Example 13, except that the host compound described in the following Table 2 was used as the host of the light-emitting layer.

The driving voltage, luminous efficiency, and the luminous color at a luminance of 1,000 nits and the time taken for luminance to decrease from 100% to 80% at a luminance of 20,000 nits (lifespan: T80) of the OLEDs of Device Examples 13 to 17 and Comparative Example 2 produced as described above, are measured, and then the results thereof are shown in the following Table 2.

TABLE 2

| | | Driving | Luminous | | |
|---|---|---|---|---|---|
| | | Voltage | Efficiency | Luminous | Lifespan |
| | Host | [V] | [cd/A] | Color | (T80) [hr] |
| Device Example 13 | 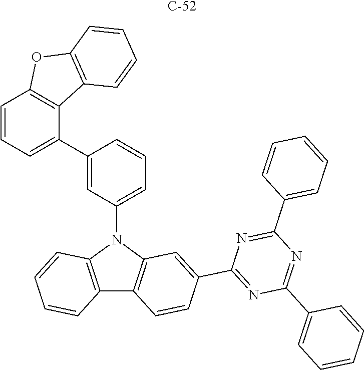 | 2.7 | 101.2 | Green | 106.0 |
| Device Example 14 | 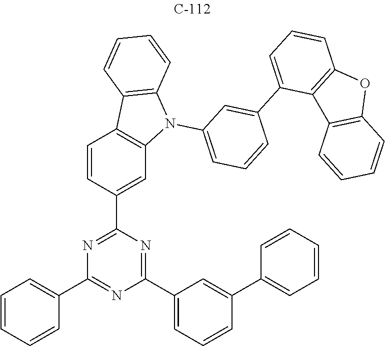 [SMILES: c1ccc(-c2cccc(-c3nc(-c4ccccc4)nc(-c4ccc5c6ccccc6n(-c6cccc(-c7cccc8oc9ccccc9c78)c6)c5c4)n3)c2)cc1] | 2.6 | 93.4 | Green | 87.2 |
| Device Example 15 | 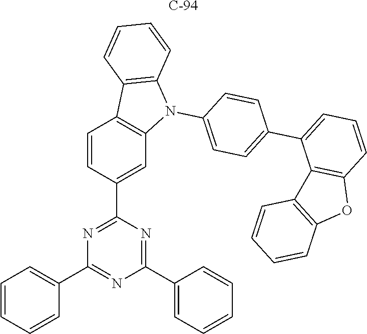 | 2.6 | 102.0 | Green | 87.2 |
| Device Example 16 | 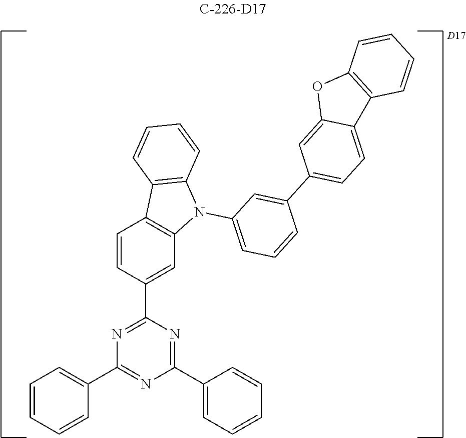 | 2.6 | 91.9 | Green | 143.5 |
| Device Example 17 | 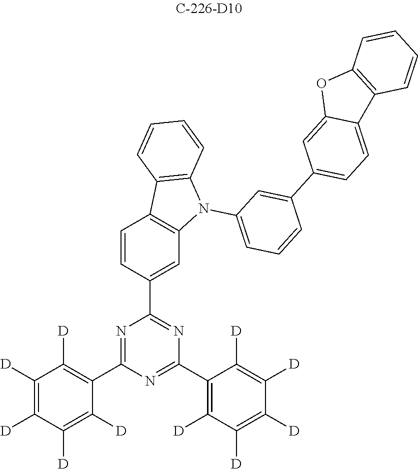 [SMILES: [2H]c1c([2H])c([2H])c(-c2nc(-c3ccc4c5ccccc5n(-c5cccc(-c6ccc7c(c6)oc6ccccc67)c5)c4c3)nc(-c3c([2H])c([2H])c([2H])c([2H])c3[2H])n2)c([2H])c1[2H]] | 2.7 | 92.6 | Green | 119.2 |
| Comparative Example 2 | 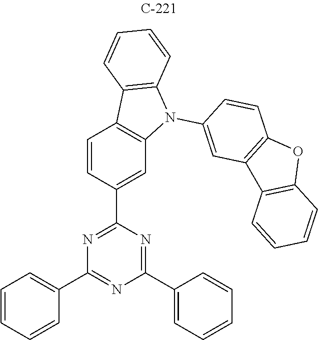 [SMILES: c1ccc(-c2nc(-c3ccccc3)nc(-c3ccc4c5ccccc5n(-c5ccc6oc7ccccc7c6c5)c4c3)n2)cc1] | 2.6 | 93.9 | Green | 69.6 |

As shown in Table 2 above, it was confirmed that the organic electroluminescent devices using an organic electroluminescent compound according to the present disclosure as a single host material (Device Examples 13 to 17) show excellent lifespan properties compared to the organic electroluminescent device comprising a conventional compound (Comparative Example 2).

### [Device Examples 18 to 21] Preparation of OLEDs Containing the Organic Electroluminescent Compound According to the Present Disclosure as a Host Material

OLEDs were manufactured in the same manner as in Device Example 1, except that the compound listed in the following Table 3 was used alone as a host material for the light emitting layer, and Compound D-150 was used as a dopant.

The driving voltage, luminous efficiency, and the luminous color at a luminance of 1,000 nits and the time taken for luminance to decrease from 100% to 80% at a luminance of 20,000 nits (lifespan: T80) of the OLEDs of Device Examples 18 to 21 produced as described above, are measured, and the results thereof are shown in the following Table 3.

TABLE 3

| | | Driving | Driving | | |
|---|---|---|---|---|---|
| | | Voltage | Voltage | Luminous | Lifespan |
| | Host | (V) | (cd/A) | Color | (T80, hr) |
| Device Example 18 | 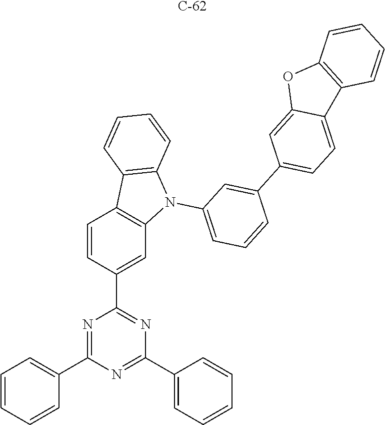 | 2.7 | 105.4 | Green | 85.9 |
| Device Example 19 | 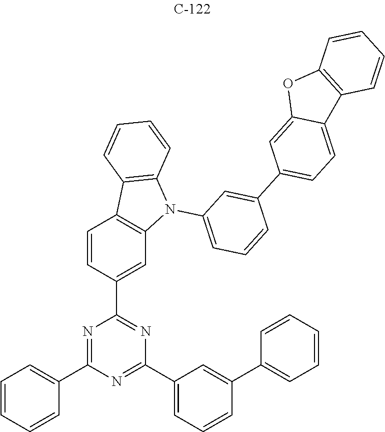 [SMILES: c1ccc(-c2cccc(-c3nc(-c4ccccc4)nc(-c4ccc5c6ccccc6n(-c6cccc(-c7ccc8c(c7)oc7ccccc78)c6)c5c4)n3)c2)cc1] | 2.6 | 104.5 | Green | 117.6 |
| Device Example 20 | 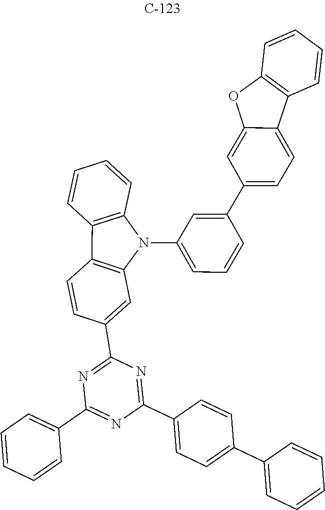 [SMILES: c1ccc(-c2ccc(-c3nc(-c4ccccc4)nc(-c4ccc5c6ccccc6n(-c6cccc(-c7ccc8c(c7)oc7ccccc78)c6)c5c4)n3)cc2)cc1] | 2.6 | 102.6 | Green | 95.7 |
| Device Example 21 | 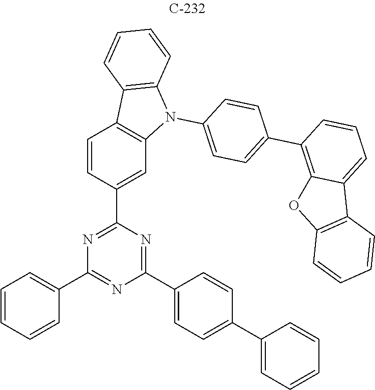 [SMILES: c1ccc(-c2ccc(-c3nc(-c4ccccc4)nc(-c4ccc5c6ccccc6n(-c6ccc(-c7cccc8c7oc7ccccc78)cc6)c5c4)n3)cc2)cc1] | 2.8 | 96.6 | Green | 361.6 |

### [Device Example 22 to 30] Preparation of Green OLEDs Deposited with a Plurality of Host Materials According to the Present Disclosure

OLEDs were manufactured in the same manner as in Device Example 1, except that each of the first host compound and the second host compound described in the following Table 4 was used as the hosts of the light-emitting layer.

### [Comparative Example 3] Preparation of an OILED Including the Comparative Compounds as Hosts

An OLED was manufactured in the same manner as in Device Example 25, except that each of the first host compound and the second host compound described in the following Table 4 was used as the hosts of the light-emitting layer.

The driving voltage, luminous efficiency, and the luminous color at a luminance of 1,000 nits and the time taken for luminance to decrease from 100% to 80% at a luminance of 60,000 nits (lifespan: T80) of the OLEDs of Device Examples 22 to 30 and Comparative Example 3 produced as described above, are measured, and then the results thereof are shown in the following Table 4.

TABLE 4

| | | | Driving | Luminous | | |
|---|---|---|---|---|---|---|
| | | | Voltage | Efficiency | Luminous | Lifespan |
| | First Host | Second Host | [V] | [cd/A] | Color | (T80) [hr] |
| Device Example 22 | 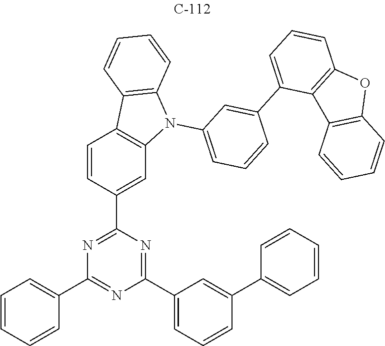 [SMILES: c1ccc(-c2cccc(-c3nc(-c4ccccc4)nc(-c4ccc5c6ccccc6n(-c6cccc(-c7cccc8oc9ccccc9c78)c6)c5c4)n3)c2)cc1] | 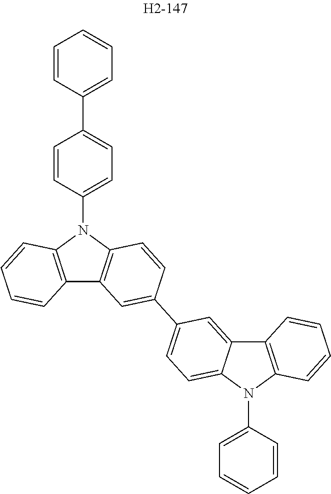 [SMILES: c1ccc(-c2ccc(-n3c4ccccc4c4cc(-c5ccc6c(c5)c5ccccc5n6-c5ccccc5)ccc43)cc2)cc1] | 3.0 | 109.6 | Green | 113.7 |
| Device Example 23 | 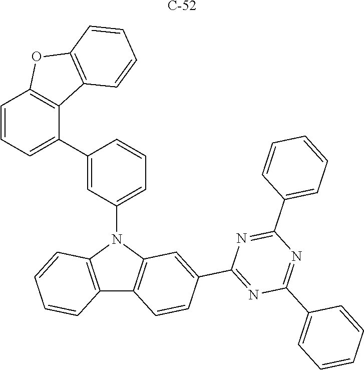 | 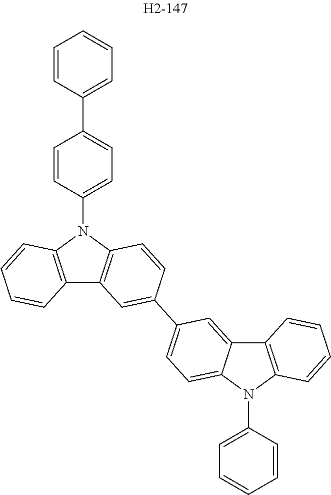 [SMILES: c1ccc(-c2ccc(-n3c4ccccc4c4cc(-c5ccc6c(c5)c5ccccc5n6-c5ccccc5)ccc43)cc2)cc1] | 3.0 | 108.6 | Green | 107.5 |
| Device Example 24 | 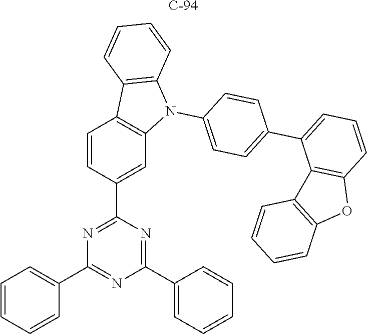 | 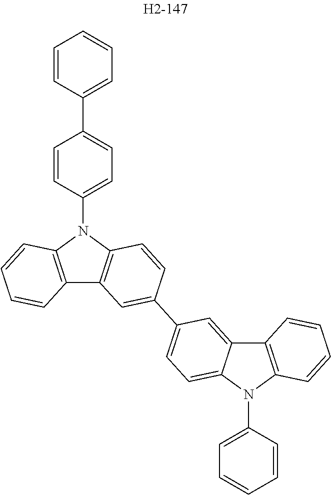 [SMILES: c1ccc(-c2ccc(-n3c4ccccc4c4cc(-c5ccc6c(c5)c5ccccc5n6-c5ccccc5)ccc43)cc2)cc1] | 2.9 | 108.6 | Green | 91.3 |
| Device Example 25 | 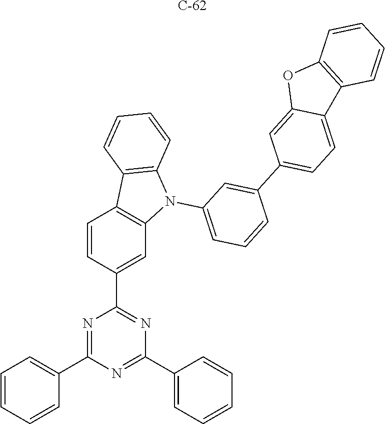 | 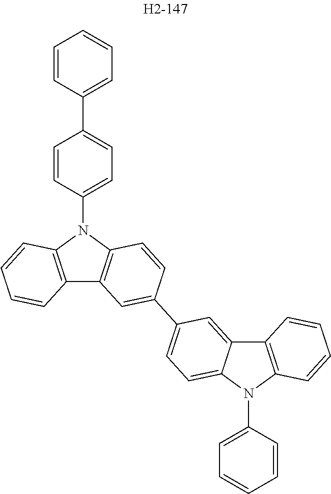 [SMILES: c1ccc(-c2ccc(-n3c4ccccc4c4cc(-c5ccc6c(c5)c5ccccc5n6-c5ccccc5)ccc43)cc2)cc1] | 3.0 | 103.6 | Green | 93.9 |
| Device Example 26 | 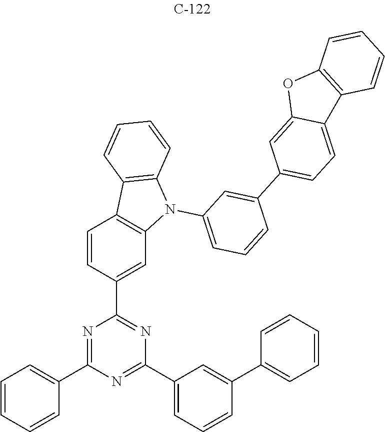 [SMILES: c1ccc(-c2cccc(-c3nc(-c4ccccc4)nc(-c4ccc5c6ccccc6n(-c6cccc(-c7ccc8c(c7)oc7ccccc78)c6)c5c4)n3)c2)cc1] | 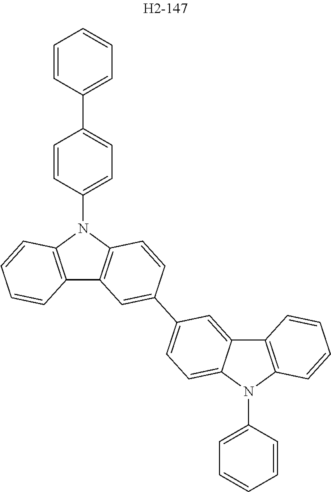 [SMILES: c1ccc(-c2ccc(-n3c4ccccc4c4cc(-c5ccc6c(c5)c5ccccc5n6-c5ccccc5)ccc43)cc2)cc1] | 3.0 | 104.8 | Green | 103.6 |
| Device Example 27 | 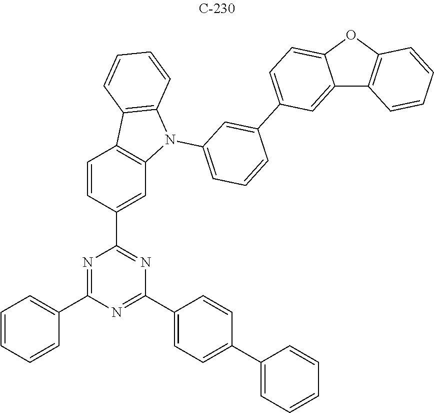 [SMILES: c1ccc(-c2ccc(-c3nc(-c4ccccc4)nc(-c4ccc5c6ccccc6n(-c6cccc(-c7ccc8oc9ccccc9c8c7)c6)c5c4)n3)cc2)cc1] | 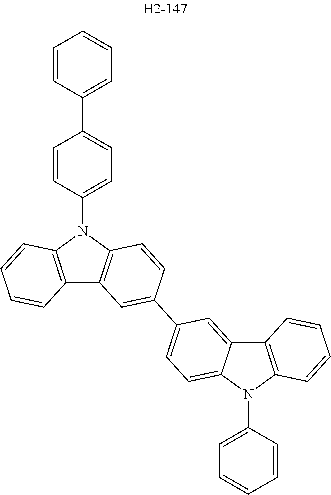 [SMILES: c1ccc(-c2ccc(-n3c4ccccc4c4cc(-c5ccc6c(c5)c5ccccc5n6-c5ccccc5)ccc43)cc2)cc1] | 2.9 | 104.9 | Green | 69.4 |
| Device Example 28 | 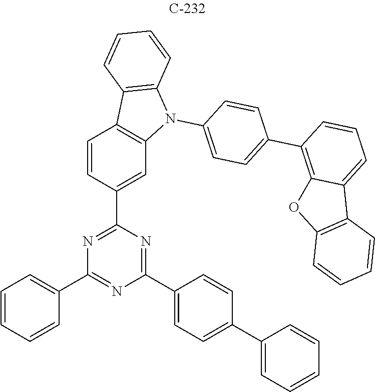 [SMILES: c1ccc(-c2ccc(-c3nc(-c4ccccc4)nc(-c4ccc5c6ccccc6n(-c6ccc(-c7cccc8c7oc7ccccc78)cc6)c5c4)n3)cc2)cc1] | 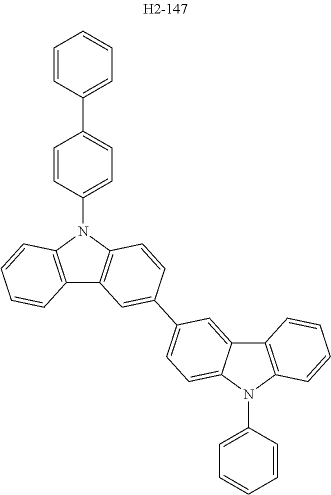 [SMILES: c1ccc(-c2ccc(-n3c4ccccc4c4cc(-c5ccc6c(c5)c5ccccc5n6-c5ccccc5)ccc43)cc2)cc1] | 3.2 | 99.0 | Green | 84.0 |
| Device Example 29 | 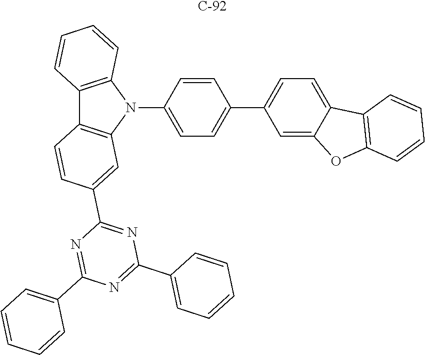 | 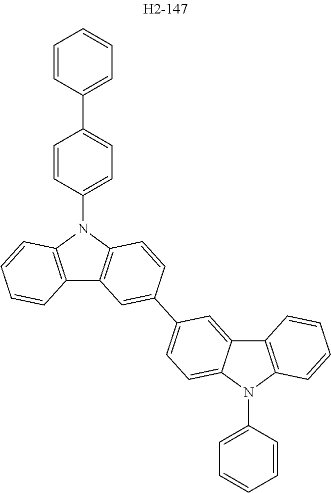 [SMILES: c1ccc(-c2ccc(-n3c4ccccc4c4cc(-c5ccc6c(c5)c5ccccc5n6-c5ccccc5)ccc43)cc2)cc1] | 2.9 | 103.1 | Green | 76.1 |
| Device Example 30 | 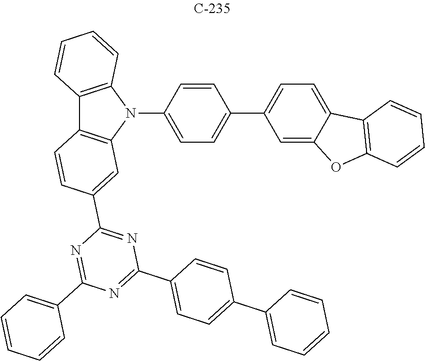 [SMILES: c1ccc(-c2ccc(-c3nc(-c4ccccc4)nc(-c4ccc5c6ccccc6n(-c6ccc(-c7ccc8c(c7)oc7ccccc78)cc6)c5c4)n3)cc2)cc1] | 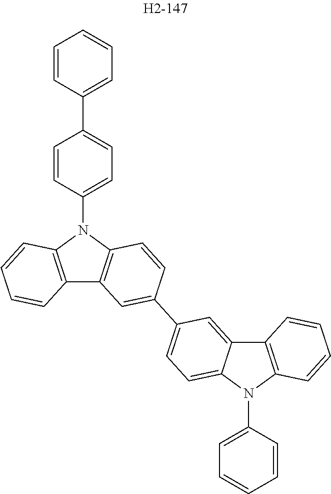 [SMILES: c1ccc(-c2ccc(-n3c4ccccc4c4cc(-c5ccc6c(c5)c5ccccc5n6-c5ccccc5)ccc43)cc2)cc1] | 2.9 | 103.0 | Green | 80.5 |
| Comparative Example 3 | 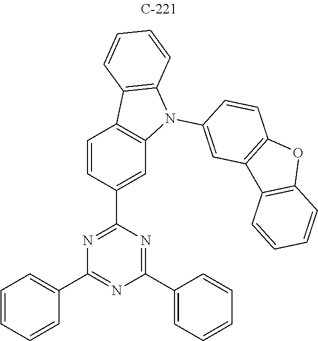 [SMILES: c1ccc(-c2nc(-c3ccccc3)nc(-c3ccc4c5ccccc5n(-c5ccc6oc7ccccc7c6c5)c4c3)n2)cc1] | 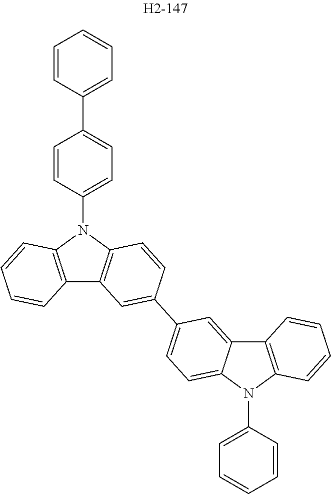 [SMILES: c1ccc(-c2ccc(-n3c4ccccc4c4cc(-c5ccc6c(c5)c5ccccc5n6-c5ccccc5)ccc43)cc2)cc1] | 2.9 | 108.2 | Green | 70.4 |

As shown in Table 4 above, it was confirmed that the organic electroluminescent devices using a plurality of host materials according to the present disclosure (Device Examples 22 to 30) show excellent lifespan properties compared to the organic electroluminescent devices comprising a conventional host combination (Comparative Example 3).

### [Device Examples 31 to 34] Preparation of Green OLEDs Deposited with a Plurality of Host Materials According to the Present Disclosure

OLEDs were manufactured in the same manner as in Device Example 1, except that each of the first host compound, the second host compound, and the third host compound (in a ratio of 1:1:1) described in the following Table 4 was used as the hosts of the light-emitting layer.

TABLE 5

| | | | | Driv- | Lumi- | | |
|---|---|---|---|---|---|---|---|
| | | | | ing | nous | Lu- | Life- |
| | | | | Volt- | Effi- | mi- | span |
| | | | | age | ciency | nous | (T80, |
| | First Host | Second Host | Third Host | (V) | (cd/A) | Color | hr) |
| De- vice Ex- am- ple 31 | 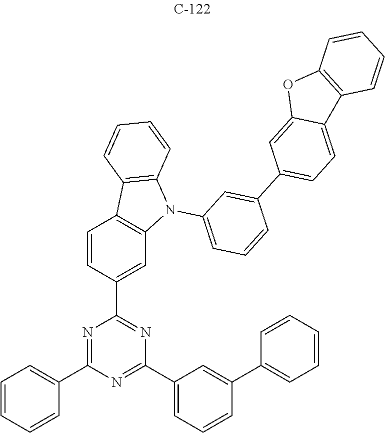 [SMILES: c1ccc(-c2cccc(-c3nc(-c4ccccc4)nc(-c4ccc5c6ccccc6n(-c6cccc(-c7ccc8c(c7)oc7ccccc78)c6)c5c4)n3)c2)cc1] | 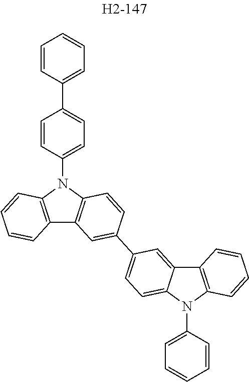 [SMILES: c1ccc(-c2ccc(-n3c4ccccc4c4cc(-c5ccc6c(c5)c5ccccc5n6-c5ccccc5)ccc43)cc2)cc1] | 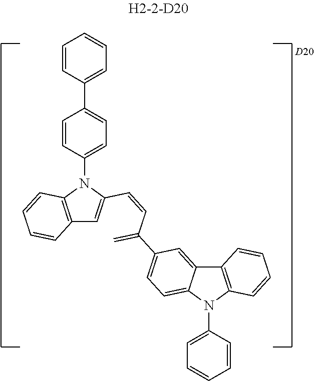 | 2.9 | 102.6 | Green | 118.5 |
| De- vice Ex- am- ple 32 | 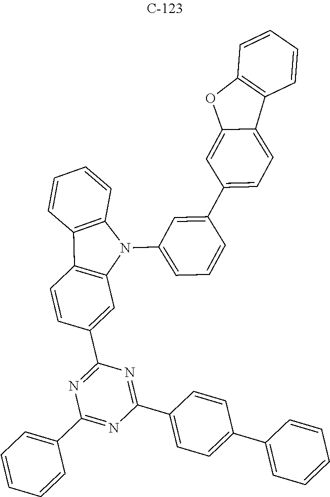 [SMILES: c1ccc(-c2ccc(-c3nc(-c4ccccc4)nc(-c4ccc5c6ccccc6n(-c6cccc(-c7ccc8c(c7)oc7ccccc78)c6)c5c4)n3)cc2)cc1] | 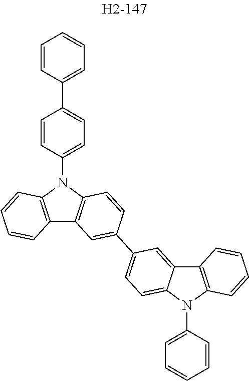 [SMILES: c1ccc(-c2ccc(-n3c4ccccc4c4cc(-c5ccc6c(c5)c5ccccc5n6-c5ccccc5)ccc43)cc2)cc1] | 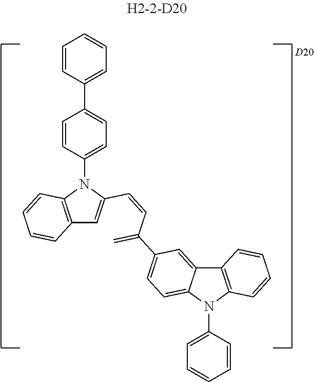 | 2.8 | 102.7 | Green | 105.1 |
| De- vice Ex- am- ple 33 | 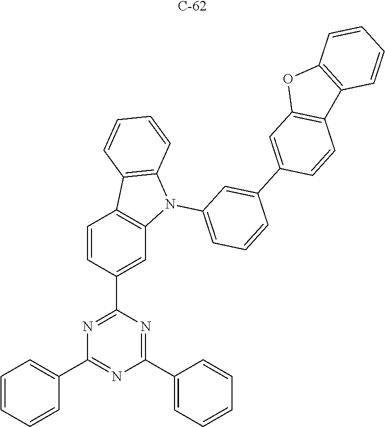 | 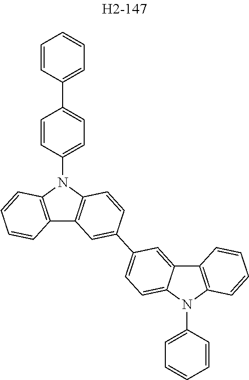 [SMILES: c1ccc(-c2ccc(-n3c4ccccc4c4cc(-c5ccc6c(c5)c5ccccc5n6-c5ccccc5)ccc43)cc2)cc1] | 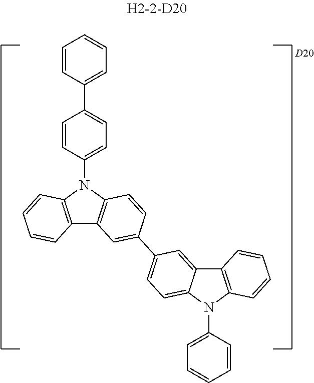 | 2.9 | 102.8 | Green | 95.4 |
| De- vice Ex- am- ple 34 | 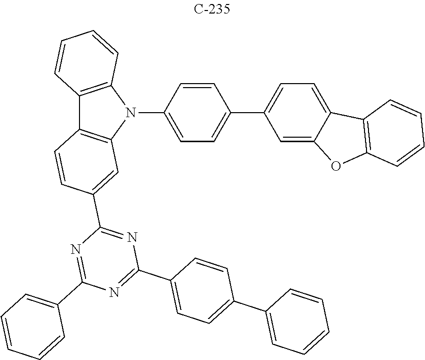 [SMILES: c1ccc(-c2ccc(-c3nc(-c4ccccc4)nc(-c4ccc5c6ccccc6n(-c6ccc(-c7ccc8c(c7)oc7ccccc78)cc6)c5c4)n3)cc2)cc1] | 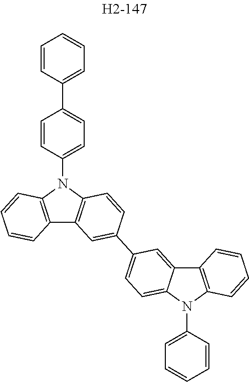 [SMILES: c1ccc(-c2ccc(-n3c4ccccc4c4cc(-c5ccc6c(c5)c5ccccc5n6-c5ccccc5)ccc43)cc2)cc1] | 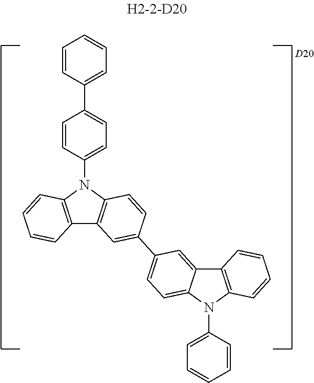 | 2.8 | 101.8 | Green | 81.3 |

The driving voltage, luminous efficiency, and the luminous color at a luminance of 1,000 nits and the time taken for luminance to decrease from 100% to 80% at a luminance of 60,000 nits (lifespan: T80) of the OLEDs of Device Examples 31 to 34 produced as described above, are measured, and the results thereof are shown in the following Table 5.

As shown in Table 5 above, an organic electroluminescent device using the same structure of compounds with a deuteriumated compound and a non-deuteriumated compound as one of the host materials of the light-emitting layer, has excellent driving voltage and/or high efficiency and/or long lifespan properties, and can be manufactured at a relatively reduced manufacturing cost.

The compounds used in Device Examples and Comparative Examples are specifically shown in the following Table 6.

TABLE 6

| | |
|---|---|
| Hole Injection Layer/ Hole Transport Layer | 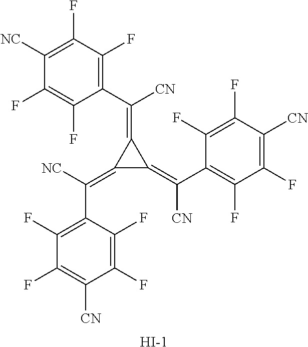 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; [I-]] |
| | 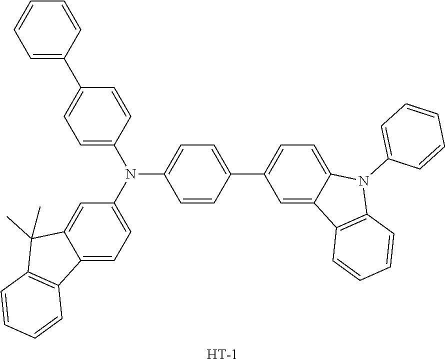 |
| | 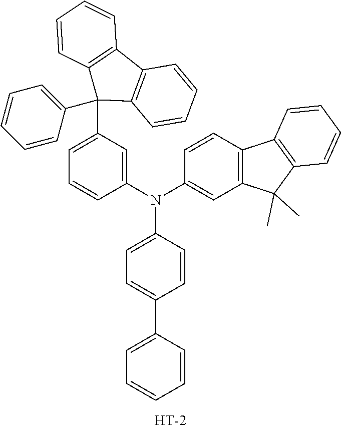 |
| Light-Emitting Layer | 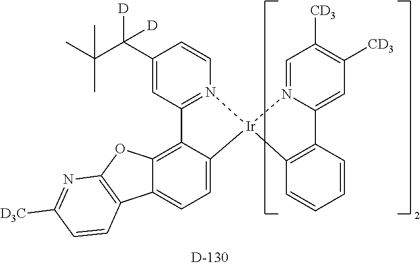 |
| | 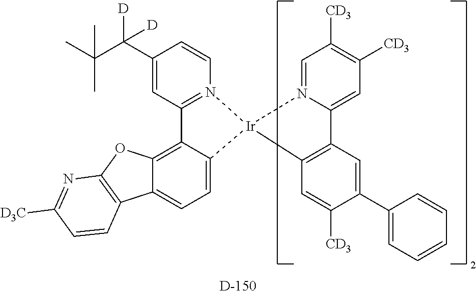 |
| Electron Transport Layer/ Electron Injection Layer | 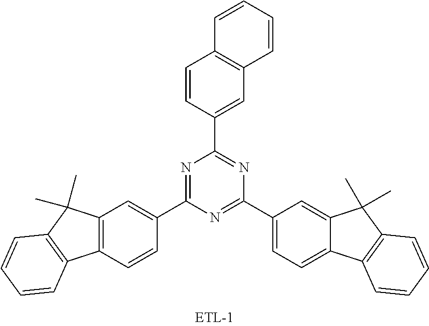 [SMILES: CC1(C)c2ccccc2-c2ccc(-c3nc(-c4ccc5c(c4)C(C)(C)c4ccccc4-5)nc(-c4ccc5ccccc5c4)n3)cc21] |
| | 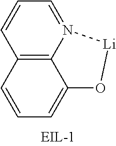 |

1. A plurality of host materials comprising at least one first host compound and at least one second host compound, wherein the first host compound is represented by the following Formula 1, the second host compound is represented by the following Formula 2, and at least one of the first host compound and the second host compound contains deuterium. 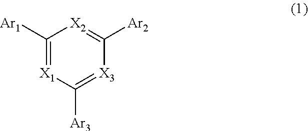
wherein,
X₁ to X₃ each independently represent N or CRₐ; provided that two of X₁ to X₃ represent N;
Rₐ represents hydrogen, deuterium, or a carbazole group represented by the following Formula 1-1;
Ar₁ to Ar₃ each independently represent hydrogen, deuterium, a substituted or unsubstituted (C6-C30)aryl, or a carbazole group represented by the following Formula 1-1;
Provided that at least one of Rₐ and Ar₁ to Ar₃ represent(s) a carbazole group represented by the following Formula 1-1; 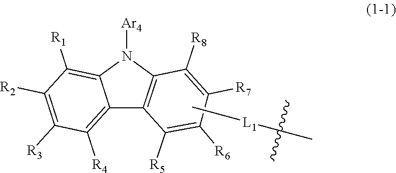
wherein,
L₁ represents a single bond, or a substituted or unsubstituted (C6-C30)arylene;
one of R₅ to R₈ is connected to L₁, and R₁ to R₄, and R₅ to R₈ not connected to L₁ each independently represent hydrogen, deuterium, a substituted or unsubstituted (C1-C30)alkyl, a substituted or unsubstituted (C6-C30)aryl, or a substituted or unsubstituted (3-to 30-membered)heteroaryl; and
Ar₄ represents a substituted or unsubstituted (C6-C30)aryl, or a substituted or unsubstituted (3- to 30-membered)heteroaryl; 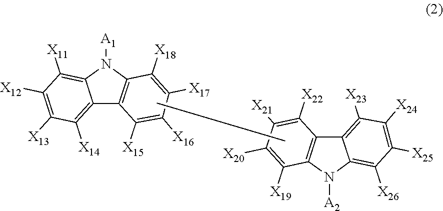
wherein,
A₁ and A₂ each independently represent a substituted or unsubstituted (C6-C30)aryl, a substituted or unsubstituted dibenzofuranyl, a substituted or unsubstituted dibenzothiophenyl, or a substituted or unsubstituted carbazolyl;
any one of X₁₅ to X₁₈ and any one of X₁₉ to X₂₂ are connected to each other to form a single bond; and
X₁₁ to X₁₄, X₂₃ to X₂₆, and X₁₅ to X₂₂, which do not form a single bond, each independently represent hydrogen, deuterium, a substituted or unsubstituted (C6-C30)aryl, or a substituted or unsubstituted (3- to 30-membered)heteroaryl; or may be linked to an adjacent substituent to form a ring(s).

2. The plurality of host materials according to claim 1, wherein (C6-C30)aryl in Ar₁ to Ar₃ of Formula 1 is each independently phenyl, biphenyl, terphenyl, quarterphenyl, naphthyl, phenylnaphthyl, naphthylphenyl, triphenylenyl, phenanthrenyl or a combination thereof, and
wherein the substituent of the substituted (C6-C30)aryl in Ar₁ to Ar₃ is at least one selected from the group consisting of deuterium and (3- to 30-membered)heteroaryl.

3. The plurality of host materials according to claim 1, wherein R₁ to R₈ in Formula 1-1 each independently represent hydrogen, deuterium, phenyl unsubstituted or substituted with deuterium, biphenyl unsubstituted or substituted with deuterium, phenanthrenyl unsubstituted or substituted with deuterium, terphenyl unsubstituted or substituted with deuterium, quarterphenyl unsubstituted or substituted with deuterium, naphthyl unsubstituted or substituted with deuterium, phenylnaphthyl unsubstituted or substituted with deuterium, naphthylphenyl unsubstituted or substituted with deuterium, dibenzofuranyl unsubstituted or substituted with deuterium, carbazolyl unsubstituted or substituted with deuterium, dibenzothiophenyl unsubstituted or substituted with deuterium, or the combination thereof.

4. The plurality of host materials according to claim 1, wherein Ar₄ in Formula 1-1 represents a substituted or unsubstituted phenyl, a substituted or unsubstituted biphenyl, a substituted or unsubstituted terphenyl, a substituted or unsubstituted dibenzofuranyl, or a substituted or unsubstituted dibenzothiophenyl, and
wherein the substituent of the substituted phenyl, the substituted biphenyl, the substituted terphenyl, the substituted dibenzofuranyl, and the substituted dibenzothiophenyl is each independently at least one selected from the group consisting of deuterium, dibenzofuranyl unsubstituted or substituted with deuterium, dibenzothiophenyl unsubstituted or substituted with deuterium, and carbazolyl unsubstituted or substituted with deuterium.

5. The plurality of host materials according to claim 1, wherein Formula 1-1 is represented by the following Formula A-1: 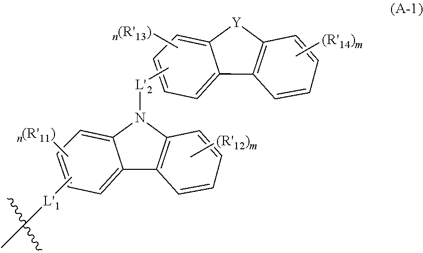
wherein,
L′₁ represents a single bond or a substituted or unsubstituted (C6-C12)arylene;
L′₂ represents a substituted or unsubstituted (C6-C12)arylene;
Y represents —O—, —S—, or —N(R′₁)—;
R′₁ represents a substituted or unsubstituted (C6-C12)aryl, or a substituted or unsubstituted dibenzofuranyl, a substituted or unsubstituted carbazolyl, or a substituted or unsubstituted dibenzothiophenyl;
R′₁₁ to R′₁₄ each independently represent hydrogen, deuterium, a substituted or unsubstituted (C6-C12)aryl, or a substituted or unsubstituted dibenzofuranyl, a substituted or unsubstituted carbazolyl, or a substituted or unsubstituted dibenzothiophenyl;
n is an integer of 1 to 3, m is an integer of 1 to 4;
when m and n are 2 or more, each of R′₁₁ to R′₁₄ may be the same or different from each other.

6. The plurality of host materials according to claim 1, wherein the first host compound does not contain deuterium, and the second host compound contains deuterium.

7. The plurality of host materials according to claim 1, wherein the deuterium substitution rate in Formula 1 is 30% to 100%.

8. The plurality of host materials according to claim 1, wherein at least one of X₁₁, X₁₈, X₁₉ and X₂₆ is(are) deuterium.

9. The plurality of host materials according to claim 1, wherein the deuterium substitution rate in Formula 2 is 40% to 100%.

10. The plurality of host materials according to claim 1, wherein the deuterium substitution rate in Formula 2 is 25% to 100%.

11. The plurality of host materials according to claim 1, wherein Formula 2 is represented by any one of the following formulas 2-1 to 2-8. 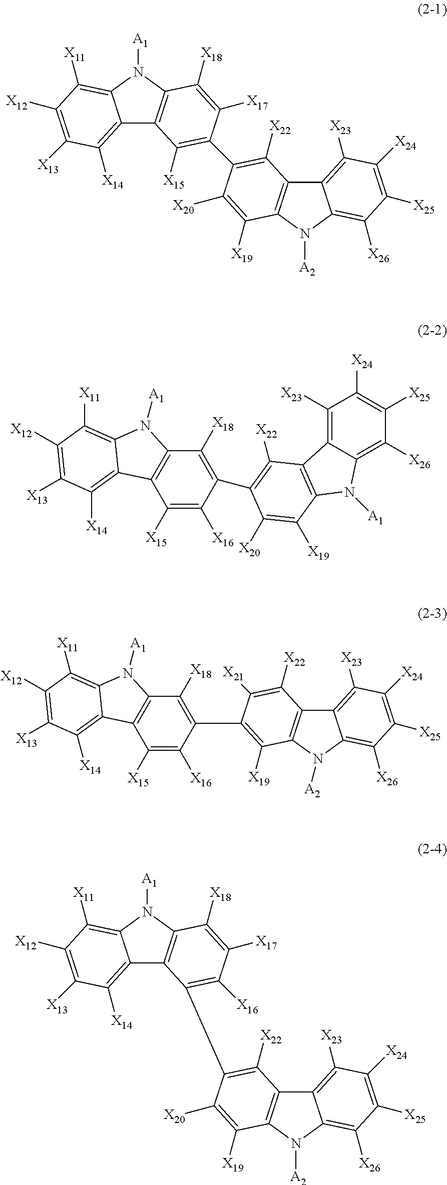 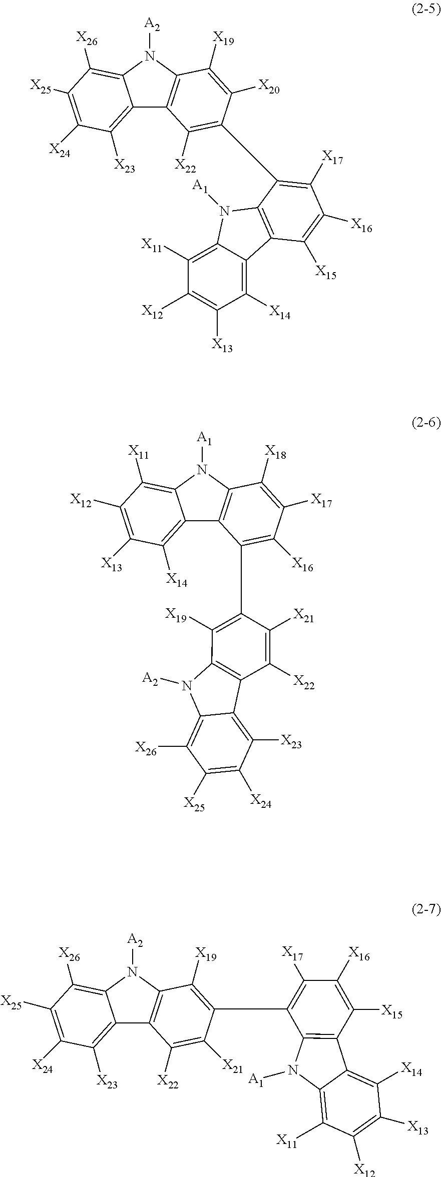 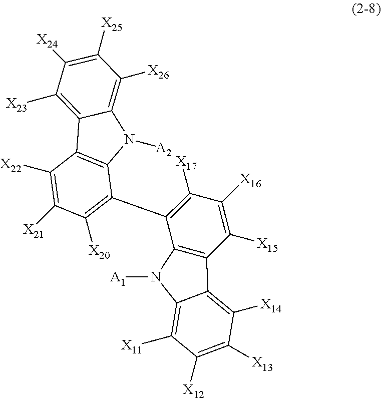
wherein,
A₁, A₂ and X₁₁ to X₂₆ are as defined in claim 1.

12. The plurality of host materials according to claim 1, wherein A₁ and A₂ in Formula 2 each independently represent phenyl unsubstituted or substituted with deuterium, biphenyl unsubstituted or substituted with deuterium, terphenyl unsubstituted or substituted with deuterium, naphthyl unsubstituted or substituted with deuterium, fluorenyl unsubstituted or substituted with at least one of deuterium (C1-C30)alkyl and (C6-C30)aryl, benzofluorenyl unsubstituted or substituted with at least one of deuterium (C1-C30)alkyl and (C6-C30)aryl, triphenylenyl unsubstituted or substituted with deuterium, fluoranthenyl unsubstituted or substituted with deuterium, phenanthrenyl unsubstituted or substituted with deuterium, dibenzofuranyl unsubstituted or substituted with deuterium, carbazolyl unsubstituted or substituted with deuterium, dibenzothiophenyl unsubstituted or substituted with deuterium, or the combination thereof.

13. The plurality of host materials according to claim 1, wherein the compound represented by Formula 1 is at least one selected from the following compounds:

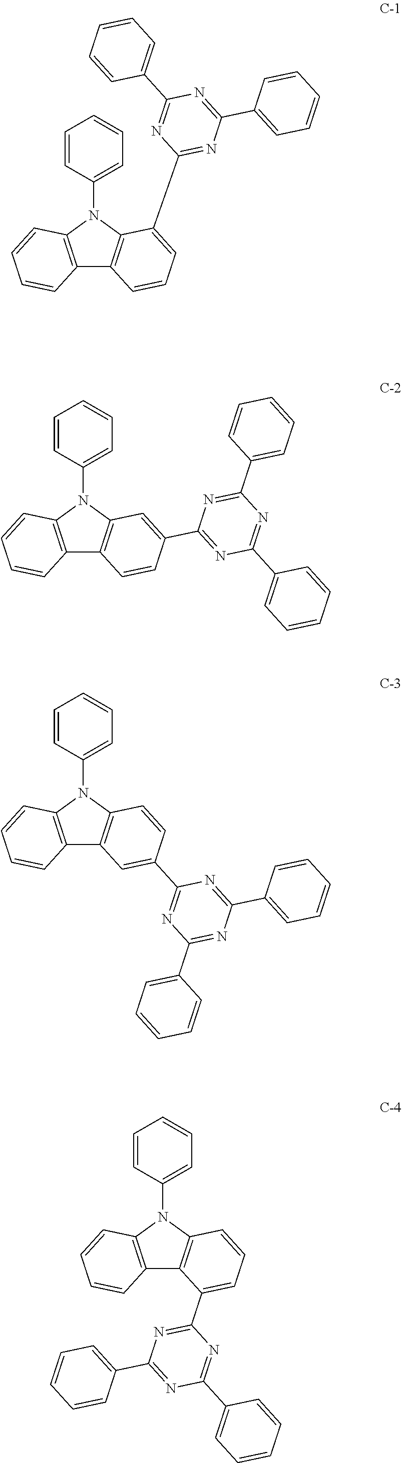 [SMILES: c1ccc(-c2nc(-c3ccccc3)nc(-c3cccc4c5ccccc5n(-c5ccccc5)c34)n2)cc1 ; c1ccc(-c2nc(-c3ccccc3)nc(-c3ccc4c5ccccc5n(-c5ccccc5)c4c3)n2)cc1 ; c1ccc(-c2nc(-c3ccccc3)nc(-c3ccc4c(c3)c3ccccc3n4-c3ccccc3)n2)cc1 ; c1ccc(-c2nc(-c3ccccc3)nc(-c3cccc4c3c3ccccc3n4-c3ccccc3)n2)cc1]

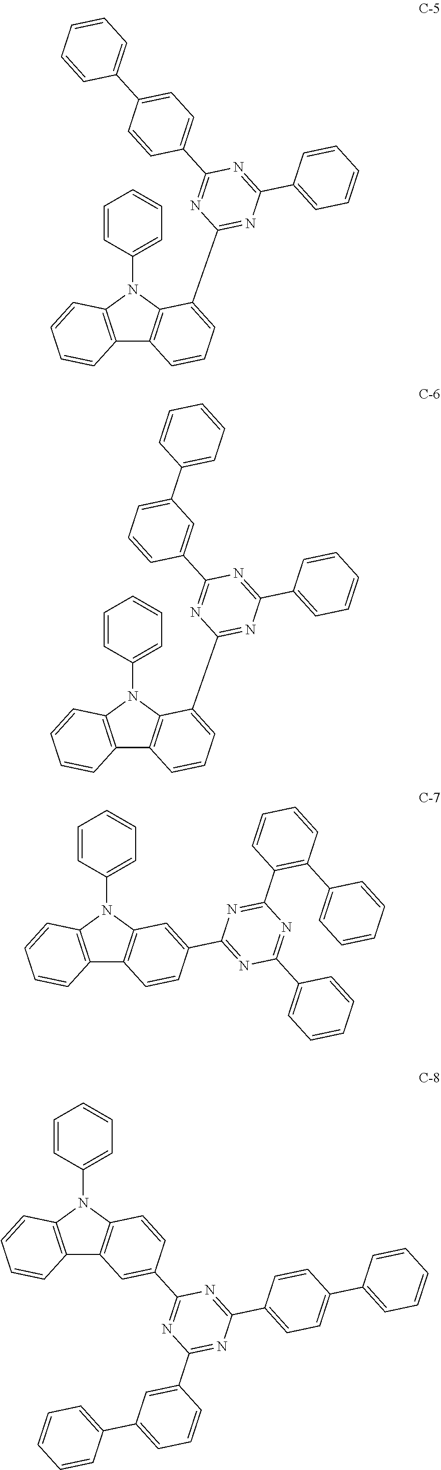

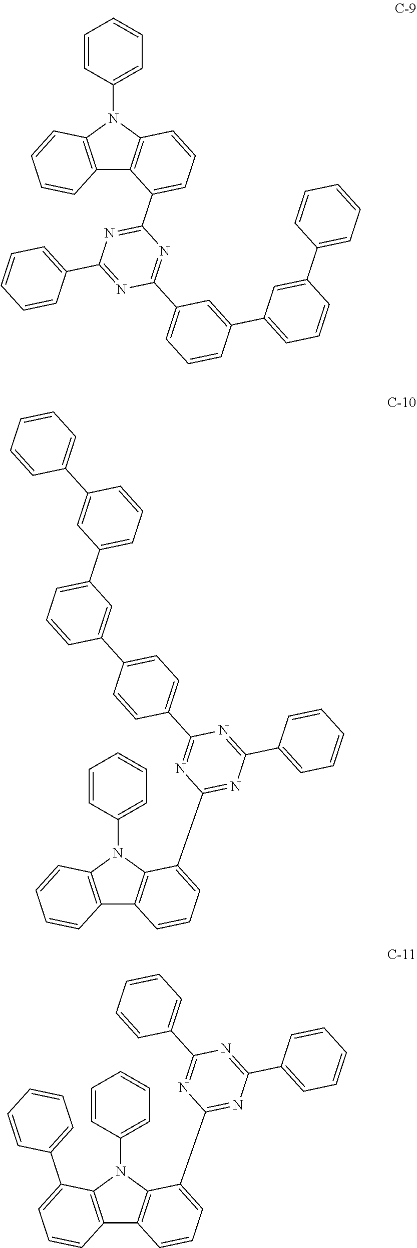

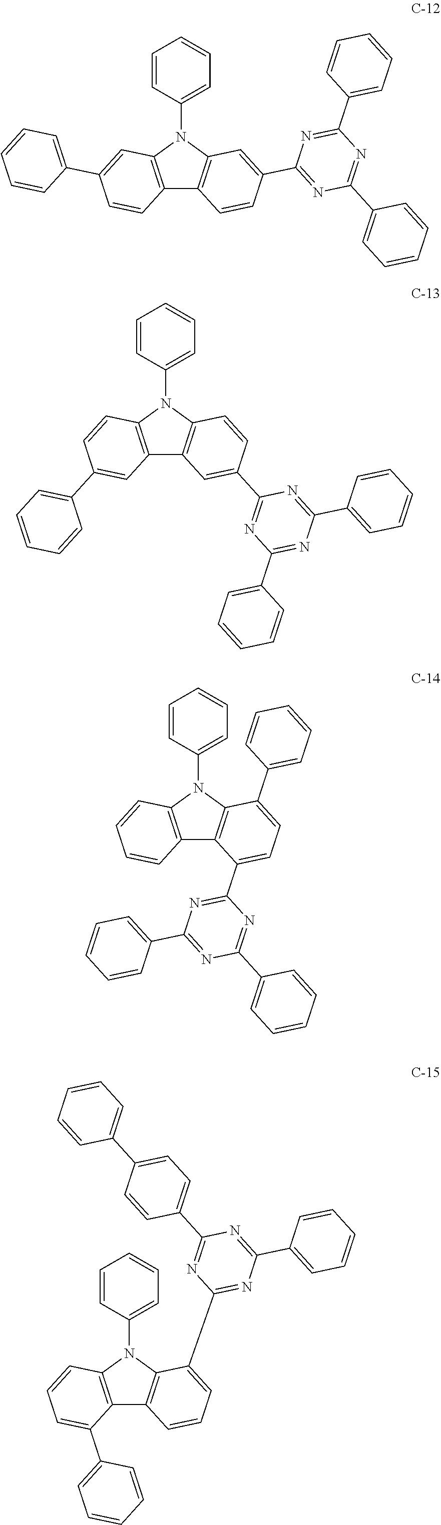 [SMILES: c1ccc(-c2ccc3c4ccc(-c5nc(-c6ccccc6)nc(-c6ccccc6)n5)cc4n(-c4ccccc4)c3c2)cc1 ; c1ccc(-c2ccc3c(c2)c2cc(-c4nc(-c5ccccc5)nc(-c5ccccc5)n4)ccc2n3-c2ccccc2)cc1 ; c1ccc(-c2nc(-c3ccccc3)nc(-c3ccc(-c4ccccc4)c4c3c3ccccc3n4-c3ccccc3)n2)cc1 ; c1ccc(-c2ccc(-c3nc(-c4ccccc4)nc(-c4cccc5c6c(-c7ccccc7)cccc6n(-c6ccccc6)c45)n3)cc2)cc1]

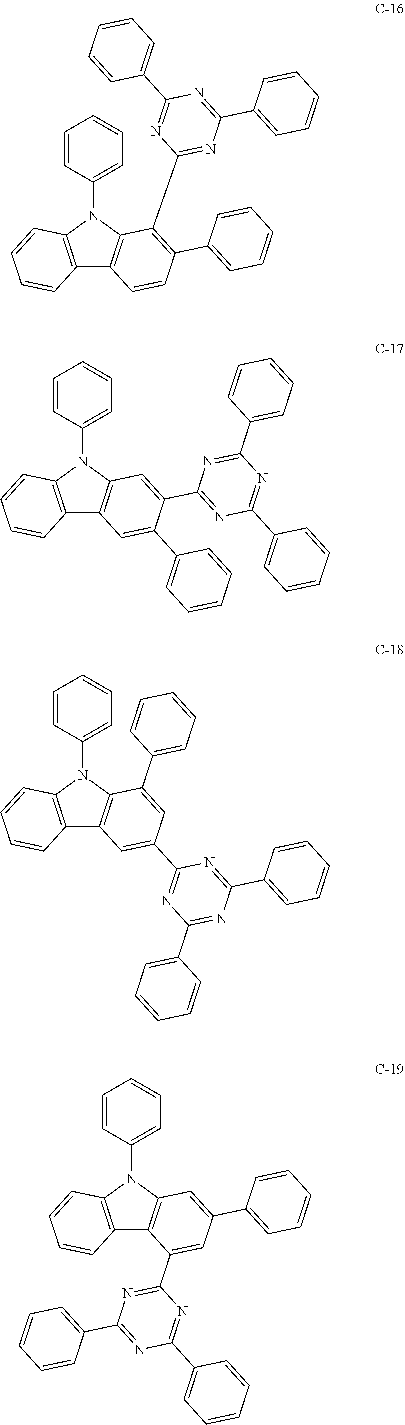 [SMILES: c1ccc(-c2nc(-c3ccccc3)nc(-c3c(-c4ccccc4)ccc4c5ccccc5n(-c5ccccc5)c34)n2)cc1 ; c1ccc(-c2nc(-c3ccccc3)nc(-c3cc4c(cc3-c3ccccc3)c3ccccc3n4-c3ccccc3)n2)cc1 ; c1ccc(-c2nc(-c3ccccc3)nc(-c3cc(-c4ccccc4)c4c(c3)c3ccccc3n4-c3ccccc3)n2)cc1 ; c1ccc(-c2cc(-c3nc(-c4ccccc4)nc(-c4ccccc4)n3)c3c4ccccc4n(-c4ccccc4)c3c2)cc1]

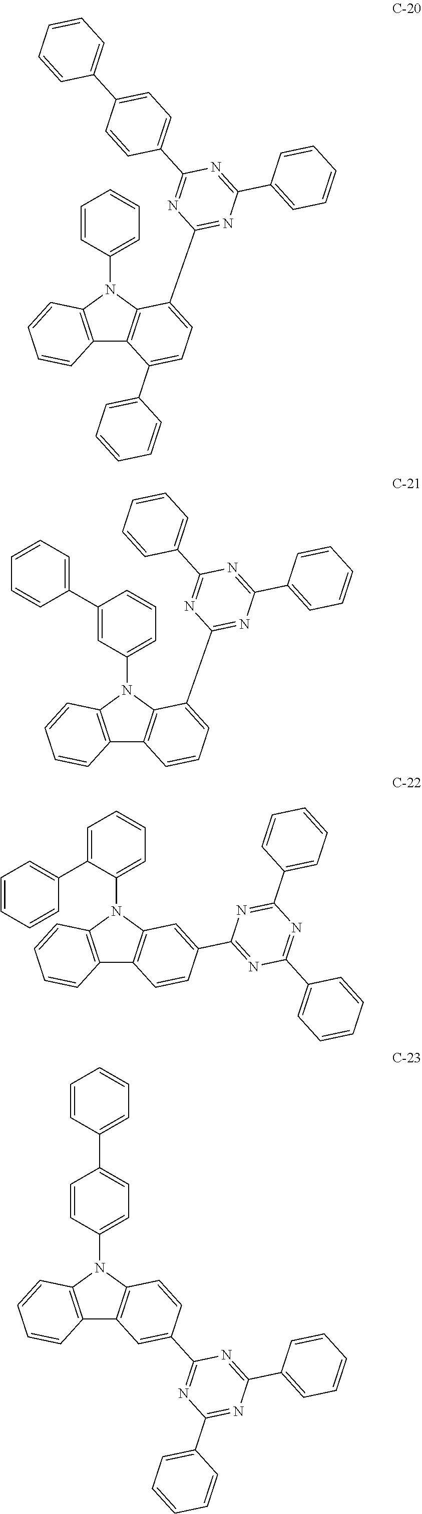

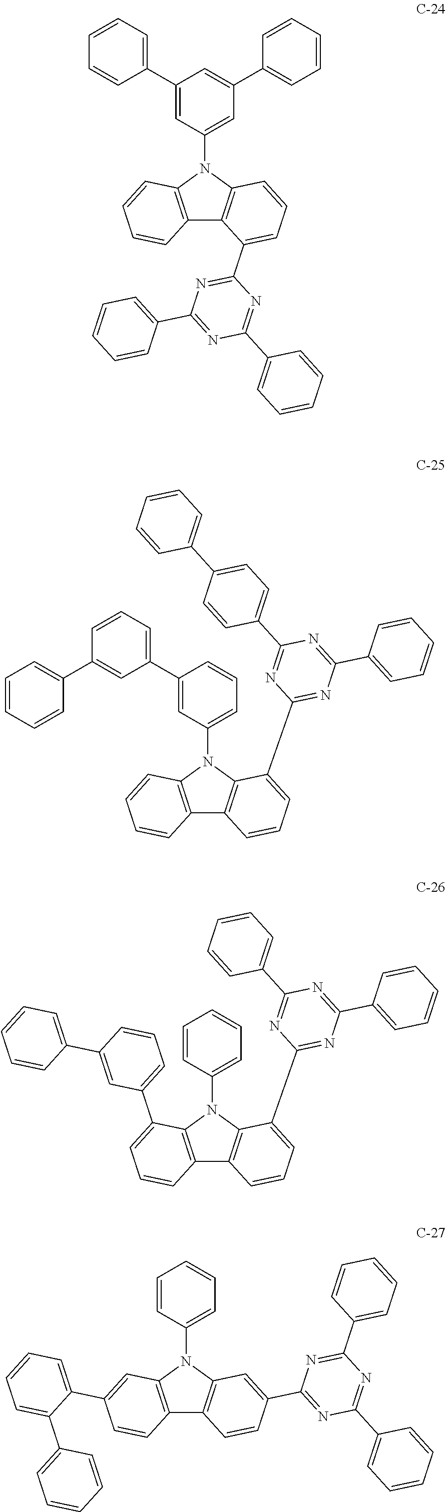 [SMILES: c1ccc(-c2cc(-c3ccccc3)cc(-n3c4ccccc4c4c(-c5nc(-c6ccccc6)nc(-c6ccccc6)n5)cccc43)c2)cc1 ; c1ccc(-c2ccc(-c3nc(-c4ccccc4)nc(-c4cccc5c6ccccc6n(-c6cccc(-c7cccc(-c8ccccc8)c7)c6)c45)n3)cc2)cc1 ; c1ccc(-c2cccc(-c3cccc4c5cccc(-c6nc(-c7ccccc7)nc(-c7ccccc7)n6)c5n(-c5ccccc5)c34)c2)cc1 ; c1ccc(-c2nc(-c3ccccc3)nc(-c3ccc4c5ccc(-c6ccccc6-c6ccccc6)cc5n(-c5ccccc5)c4c3)n2)cc1]

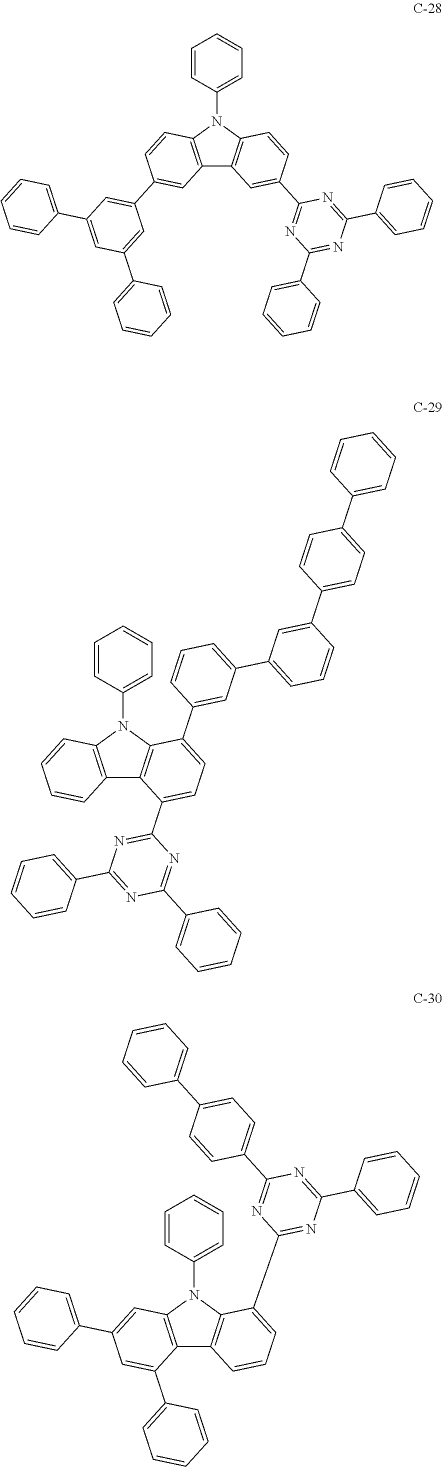 [SMILES: c1ccc(-c2cc(-c3ccccc3)cc(-c3ccc4c(c3)c3cc(-c5nc(-c6ccccc6)nc(-c6ccccc6)n5)ccc3n4-c3ccccc3)c2)cc1 ; c1ccc(-c2ccc(-c3cccc(-c4cccc(-c5ccc(-c6nc(-c7ccccc7)nc(-c7ccccc7)n6)c6c7ccccc7n(-c7ccccc7)c56)c4)c3)cc2)cc1 ; c1ccc(-c2ccc(-c3nc(-c4ccccc4)nc(-c4cccc5c6c(-c7ccccc7)cc(-c7ccccc7)cc6n(-c6ccccc6)c45)n3)cc2)cc1]

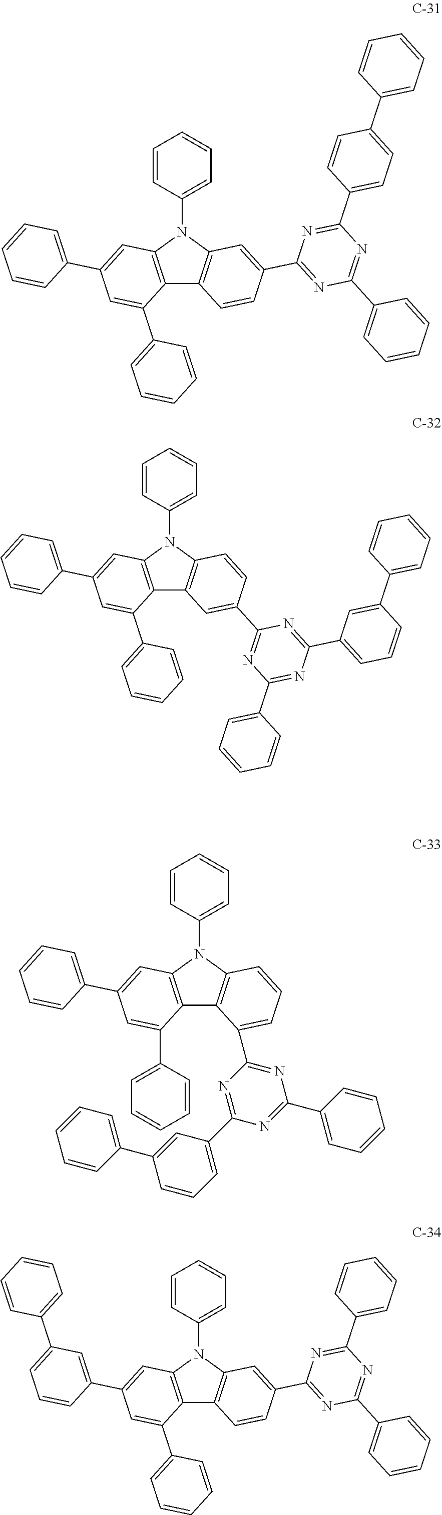

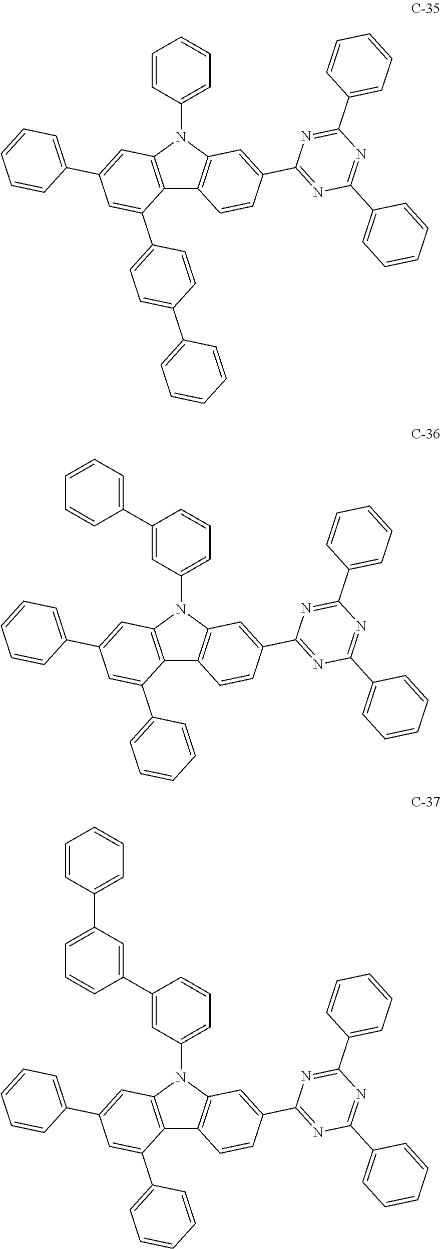 [SMILES: c1ccc(-c2ccc(-c3cc(-c4ccccc4)cc4c3c3ccc(-c5nc(-c6ccccc6)nc(-c6ccccc6)n5)cc3n4-c3ccccc3)cc2)cc1 ; c1ccc(-c2cccc(-n3c4cc(-c5nc(-c6ccccc6)nc(-c6ccccc6)n5)ccc4c4c(-c5ccccc5)cc(-c5ccccc5)cc43)c2)cc1 ; c1ccc(-c2cccc(-c3cccc(-n4c5cc(-c6nc(-c7ccccc7)nc(-c7ccccc7)n6)ccc5c5c(-c6ccccc6)cc(-c6ccccc6)cc54)c3)c2)cc1]

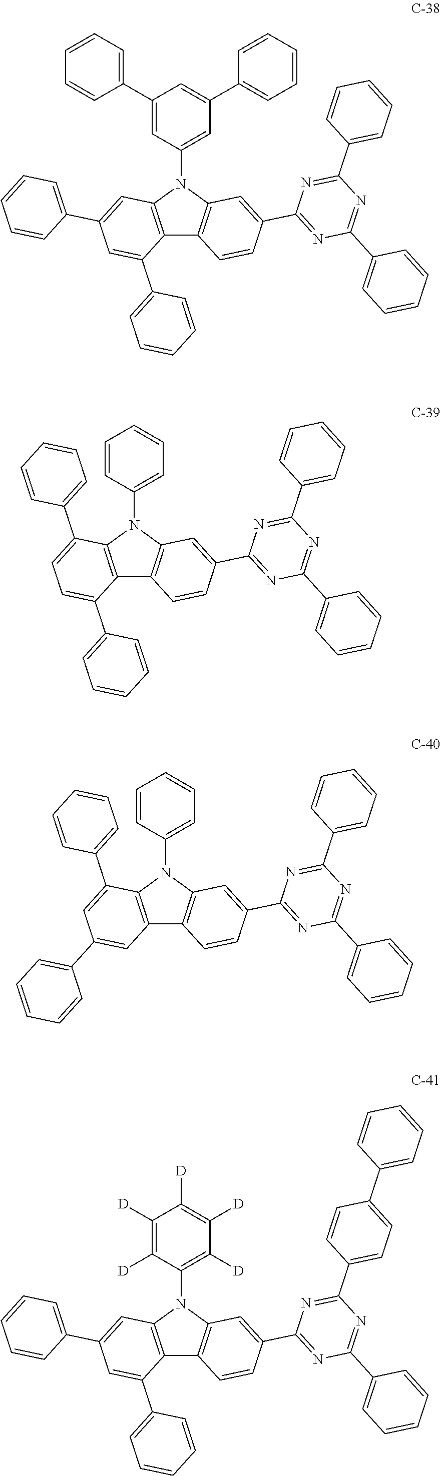 [SMILES: c1ccc(-c2cc(-c3ccccc3)cc(-n3c4cc(-c5nc(-c6ccccc6)nc(-c6ccccc6)n5)ccc4c4c(-c5ccccc5)cc(-c5ccccc5)cc43)c2)cc1 ; c1ccc(-c2nc(-c3ccccc3)nc(-c3ccc4c5c(-c6ccccc6)ccc(-c6ccccc6)c5n(-c5ccccc5)c4c3)n2)cc1 ; c1ccc(-c2cc(-c3ccccc3)c3c(c2)c2ccc(-c4nc(-c5ccccc5)nc(-c5ccccc5)n4)cc2n3-c2ccccc2)cc1 ; [2H]c1c([2H])c([2H])c(-n2c3cc(-c4nc(-c5ccccc5)nc(-c5ccc(-c6ccccc6)cc5)n4)ccc3c3c(-c4ccccc4)cc(-c4ccccc4)cc32)c([2H])c1[2H]]

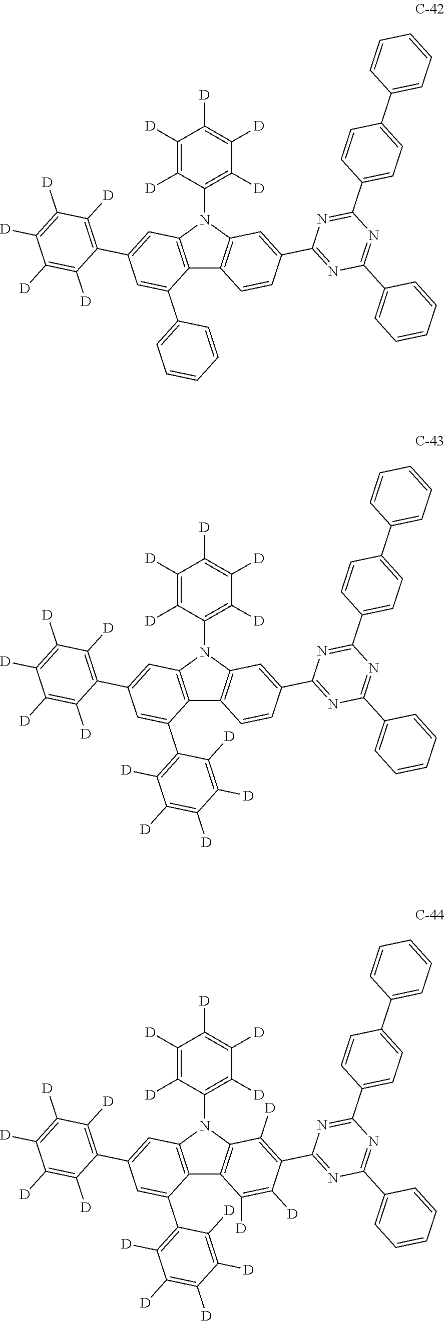

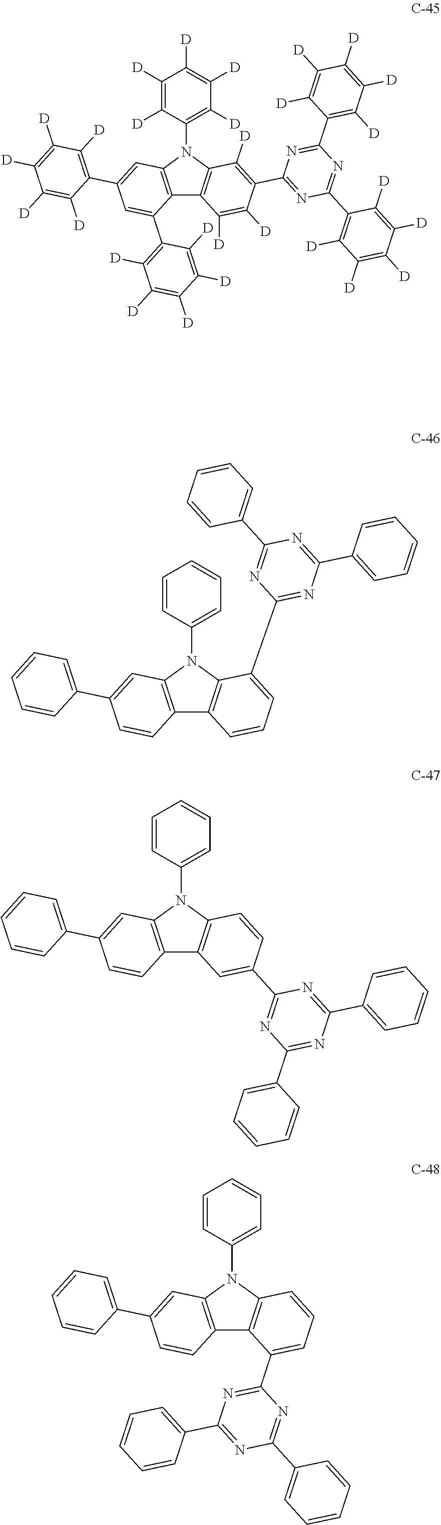

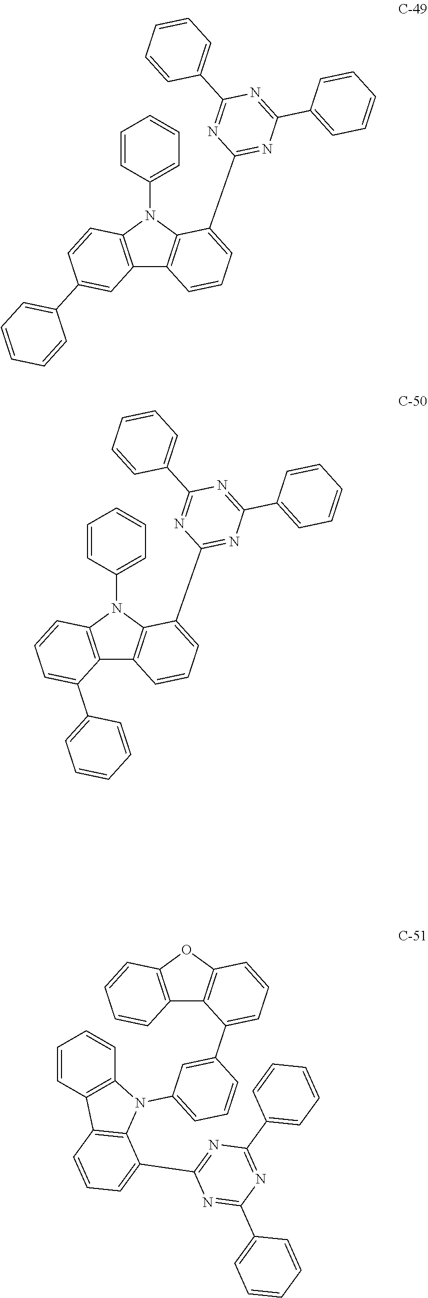 [SMILES: c1ccc(-c2ccc3c(c2)c2cccc(-c4nc(-c5ccccc5)nc(-c5ccccc5)n4)c2n3-c2ccccc2)cc1 ; c1ccc(-c2nc(-c3ccccc3)nc(-c3cccc4c5c(-c6ccccc6)cccc5n(-c5ccccc5)c34)n2)cc1 ; c1ccc(-c2nc(-c3ccccc3)nc(-c3cccc4c5ccccc5n(-c5cccc(-c6cccc7oc8ccccc8c67)c5)c34)n2)cc1]

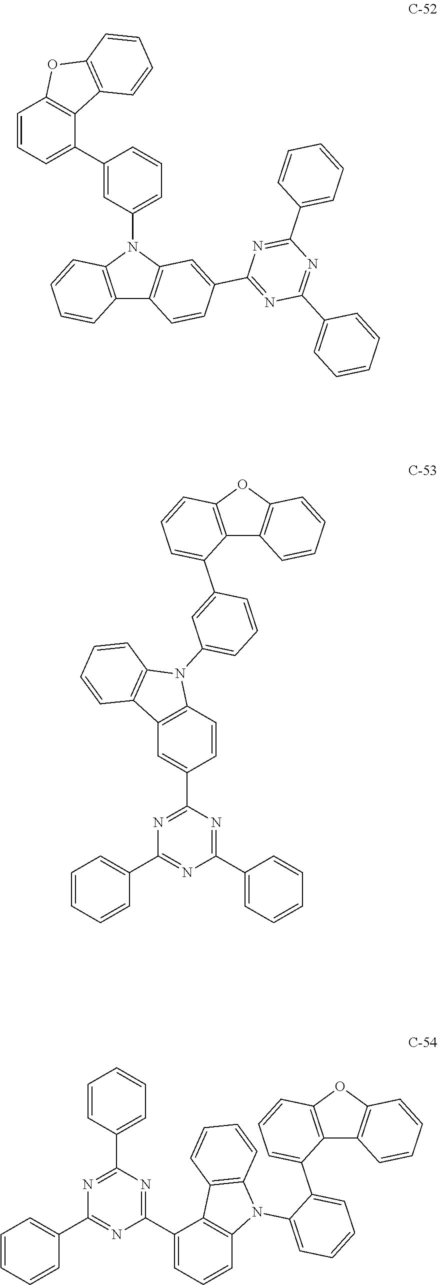 [SMILES: c1ccc(-c2nc(-c3ccccc3)nc(-c3ccc4c5ccccc5n(-c5cccc(-c6cccc7oc8ccccc8c67)c5)c4c3)n2)cc1 ; c1ccc(-c2nc(-c3ccccc3)nc(-c3ccc4c(c3)c3ccccc3n4-c3cccc(-c4cccc5oc6ccccc6c45)c3)n2)cc1 ; c1ccc(-c2nc(-c3ccccc3)nc(-c3cccc4c3c3ccccc3n4-c3ccccc3-c3cccc4oc5ccccc5c34)n2)cc1]

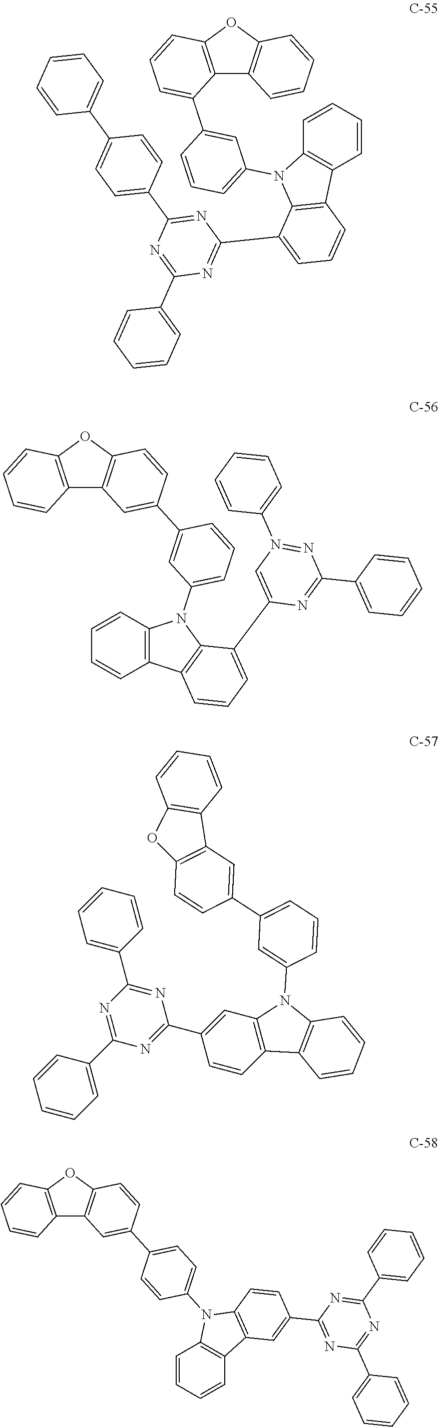

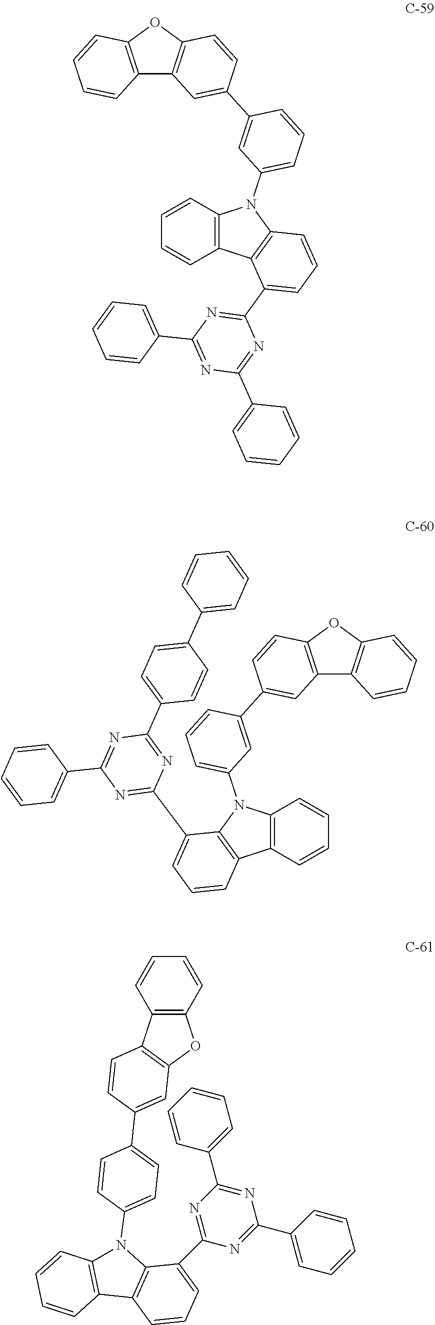 [SMILES: c1ccc(-c2nc(-c3ccccc3)nc(-c3cccc4c3c3ccccc3n4-c3cccc(-c4ccc5oc6ccccc6c5c4)c3)n2)cc1 ; c1ccc(-c2ccc(-c3nc(-c4ccccc4)nc(-c4cccc5c6ccccc6n(-c6cccc(-c7ccc8oc9ccccc9c8c7)c6)c45)n3)cc2)cc1 ; c1ccc(-c2nc(-c3ccccc3)nc(-c3cccc4c5ccccc5n(-c5ccc(-c6ccc7c(c6)oc6ccccc67)cc5)c34)n2)cc1]

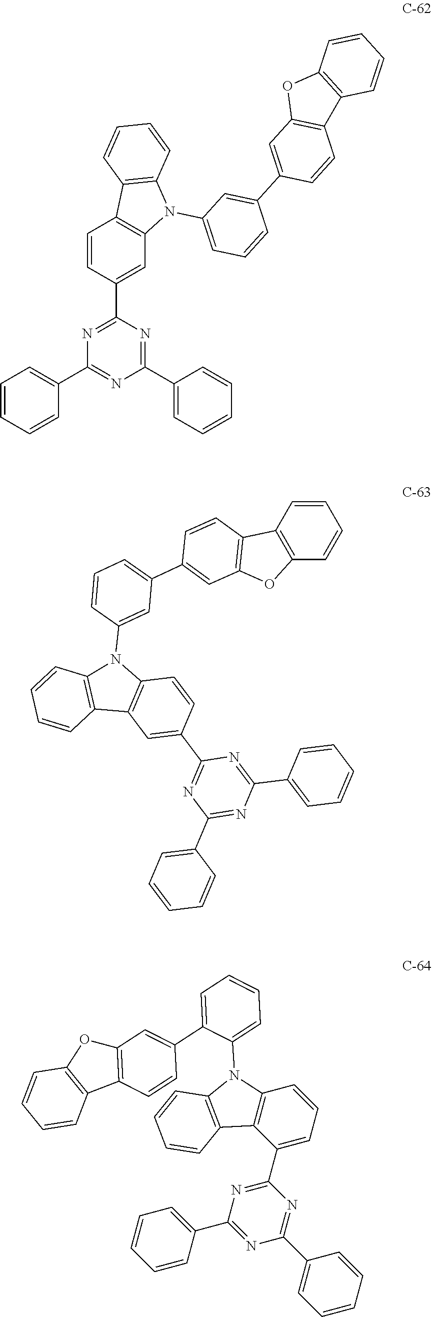 [SMILES: c1ccc(-c2nc(-c3ccccc3)nc(-c3ccc4c5ccccc5n(-c5cccc(-c6ccc7c(c6)oc6ccccc67)c5)c4c3)n2)cc1 ; c1ccc(-c2nc(-c3ccccc3)nc(-c3ccc4c(c3)c3ccccc3n4-c3cccc(-c4ccc5c(c4)oc4ccccc45)c3)n2)cc1 ; c1ccc(-c2nc(-c3ccccc3)nc(-c3cccc4c3c3ccccc3n4-c3ccccc3-c3ccc4c(c3)oc3ccccc34)n2)cc1]

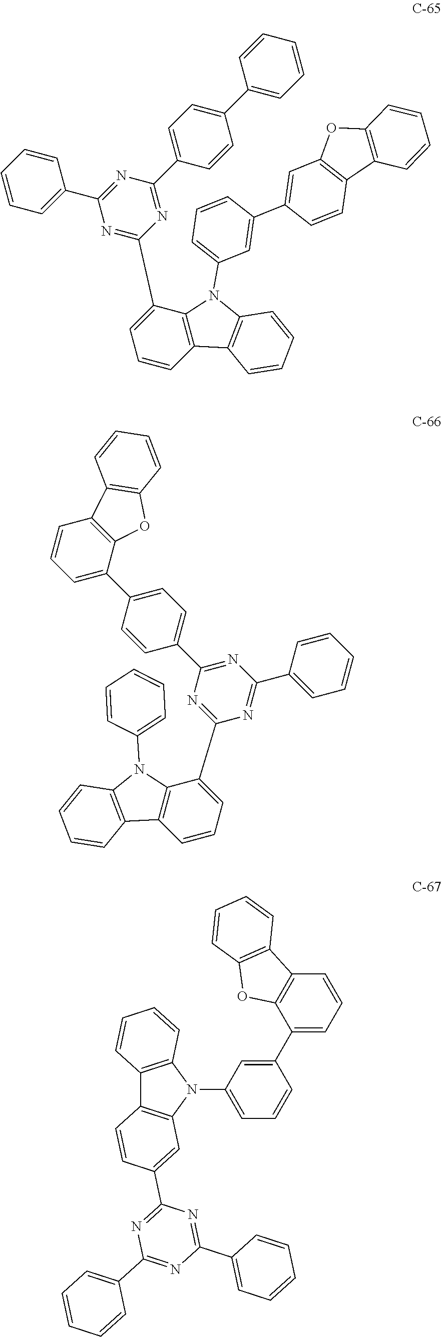 [SMILES: c1ccc(-c2ccc(-c3nc(-c4ccccc4)nc(-c4cccc5c6ccccc6n(-c6cccc(-c7ccc8c(c7)oc7ccccc78)c6)c45)n3)cc2)cc1 ; c1ccc(-c2nc(-c3ccc(-c4cccc5c4oc4ccccc45)cc3)nc(-c3cccc4c5ccccc5n(-c5ccccc5)c34)n2)cc1 ; c1ccc(-c2nc(-c3ccccc3)nc(-c3ccc4c5ccccc5n(-c5cccc(-c6cccc7c6oc6ccccc67)c5)c4c3)n2)cc1]

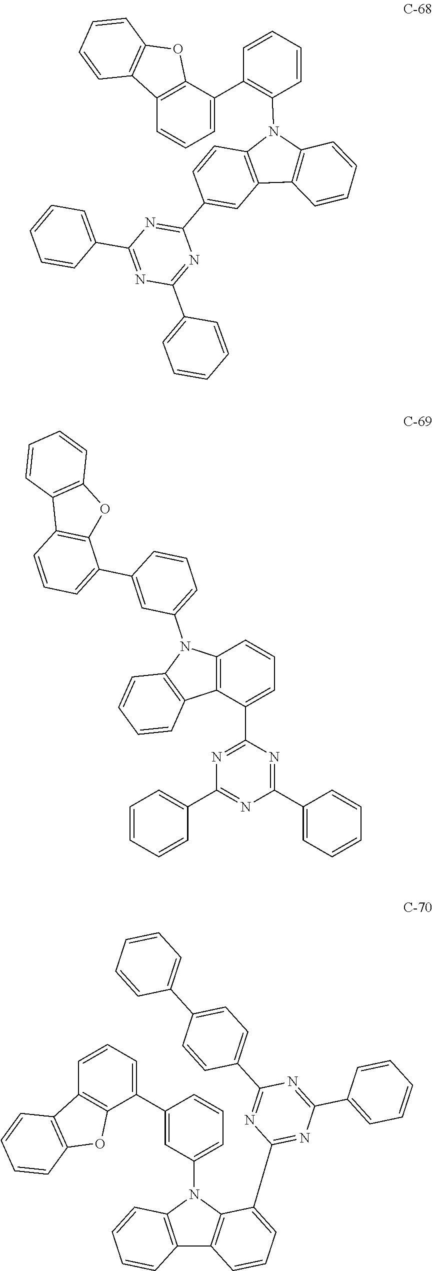 [SMILES: c1ccc(-c2nc(-c3ccccc3)nc(-c3ccc4c(c3)c3ccccc3n4-c3ccccc3-c3cccc4c3oc3ccccc34)n2)cc1 ; c1ccc(-c2nc(-c3ccccc3)nc(-c3cccc4c3c3ccccc3n4-c3cccc(-c4cccc5c4oc4ccccc45)c3)n2)cc1 ; c1ccc(-c2ccc(-c3nc(-c4ccccc4)nc(-c4cccc5c6ccccc6n(-c6cccc(-c7cccc8c7oc7ccccc78)c6)c45)n3)cc2)cc1]

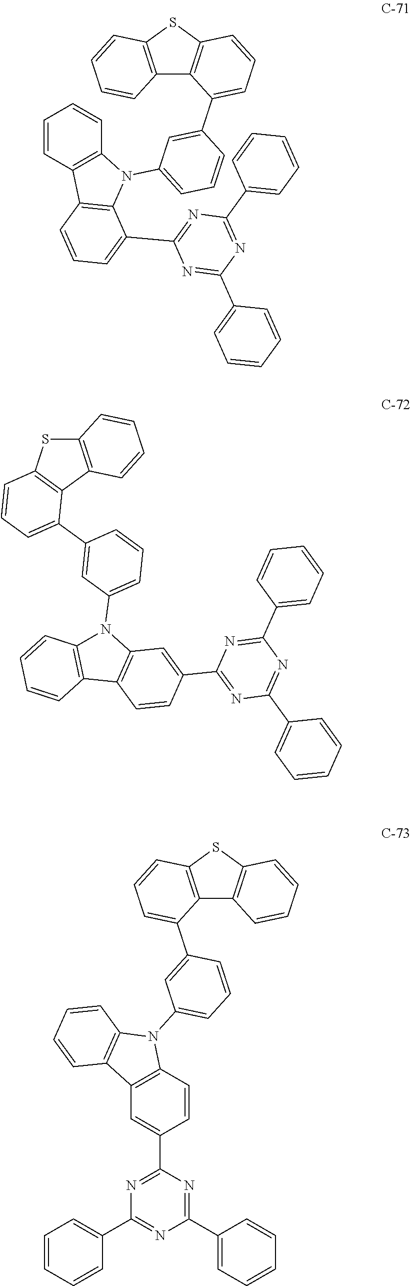 [SMILES: c1ccc(-c2nc(-c3ccccc3)nc(-c3cccc4c5ccccc5n(-c5cccc(-c6cccc7sc8ccccc8c67)c5)c34)n2)cc1 ; c1ccc(-c2nc(-c3ccccc3)nc(-c3ccc4c5ccccc5n(-c5cccc(-c6cccc7sc8ccccc8c67)c5)c4c3)n2)cc1 ; c1ccc(-c2nc(-c3ccccc3)nc(-c3ccc4c(c3)c3ccccc3n4-c3cccc(-c4cccc5sc6ccccc6c45)c3)n2)cc1]

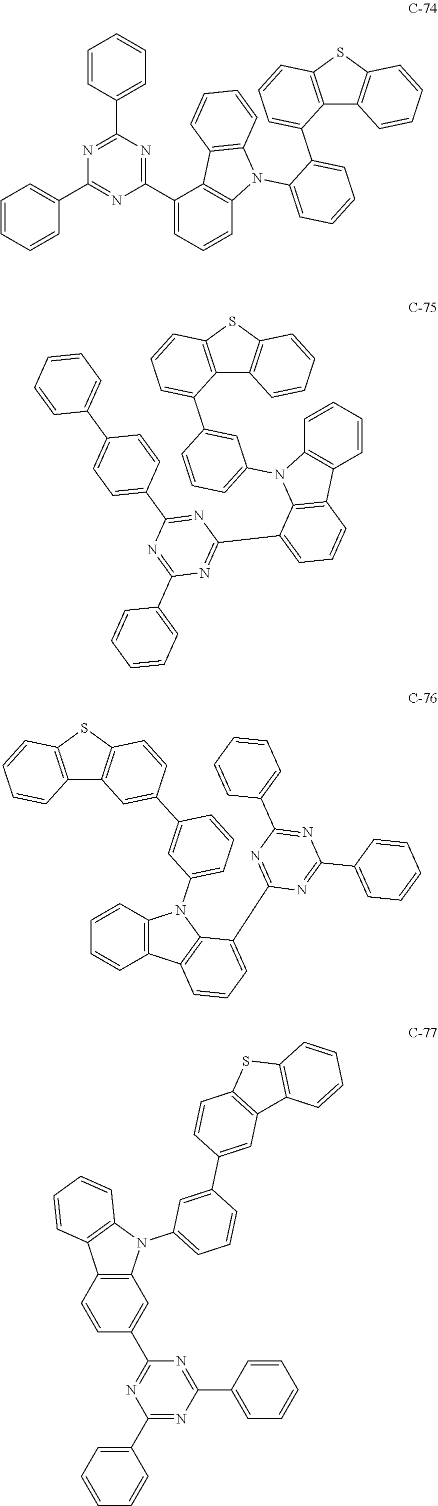

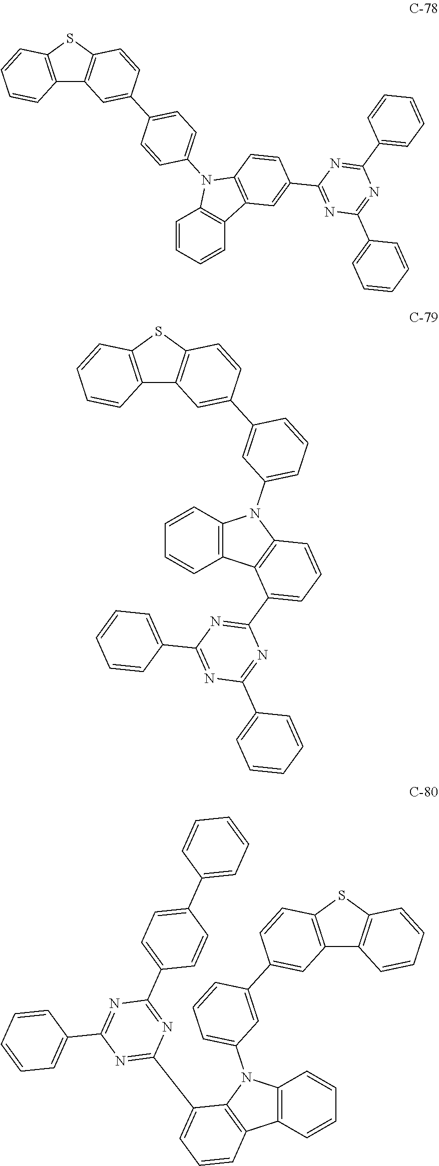 [SMILES: c1ccc(-c2nc(-c3ccccc3)nc(-c3ccc4c(c3)c3ccccc3n4-c3ccc(-c4ccc5sc6ccccc6c5c4)cc3)n2)cc1 ; c1ccc(-c2nc(-c3ccccc3)nc(-c3cccc4c3c3ccccc3n4-c3cccc(-c4ccc5sc6ccccc6c5c4)c3)n2)cc1 ; c1ccc(-c2ccc(-c3nc(-c4ccccc4)nc(-c4cccc5c6ccccc6n(-c6cccc(-c7ccc8sc9ccccc9c8c7)c6)c45)n3)cc2)cc1]

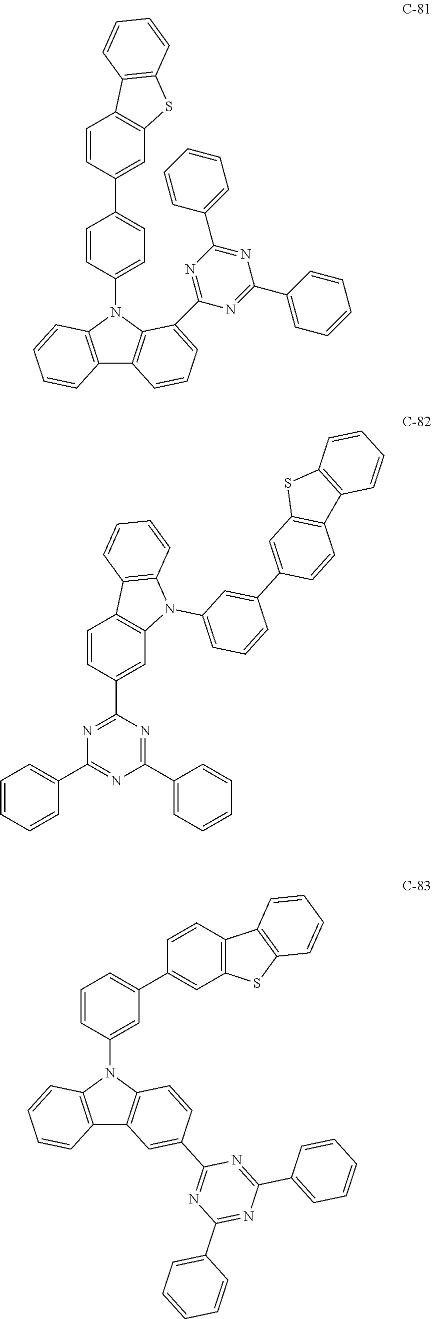 [SMILES: c1ccc(-c2nc(-c3ccccc3)nc(-c3cccc4c5ccccc5n(-c5ccc(-c6ccc7c(c6)sc6ccccc67)cc5)c34)n2)cc1 ; c1ccc(-c2nc(-c3ccccc3)nc(-c3ccc4c5ccccc5n(-c5cccc(-c6ccc7c(c6)sc6ccccc67)c5)c4c3)n2)cc1 ; c1ccc(-c2nc(-c3ccccc3)nc(-c3ccc4c(c3)c3ccccc3n4-c3cccc(-c4ccc5c(c4)sc4ccccc45)c3)n2)cc1]

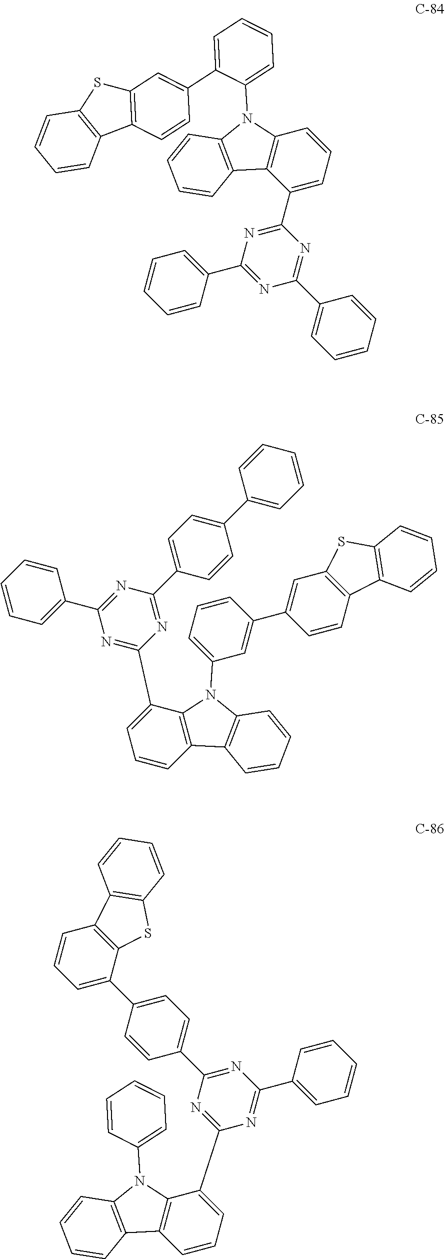 [SMILES: c1ccc(-c2nc(-c3ccccc3)nc(-c3cccc4c3c3ccccc3n4-c3ccccc3-c3ccc4c(c3)sc3ccccc34)n2)cc1 ; c1ccc(-c2ccc(-c3nc(-c4ccccc4)nc(-c4cccc5c6ccccc6n(-c6cccc(-c7ccc8c(c7)sc7ccccc78)c6)c45)n3)cc2)cc1 ; c1ccc(-c2nc(-c3ccc(-c4cccc5c4sc4ccccc45)cc3)nc(-c3cccc4c5ccccc5n(-c5ccccc5)c34)n2)cc1]

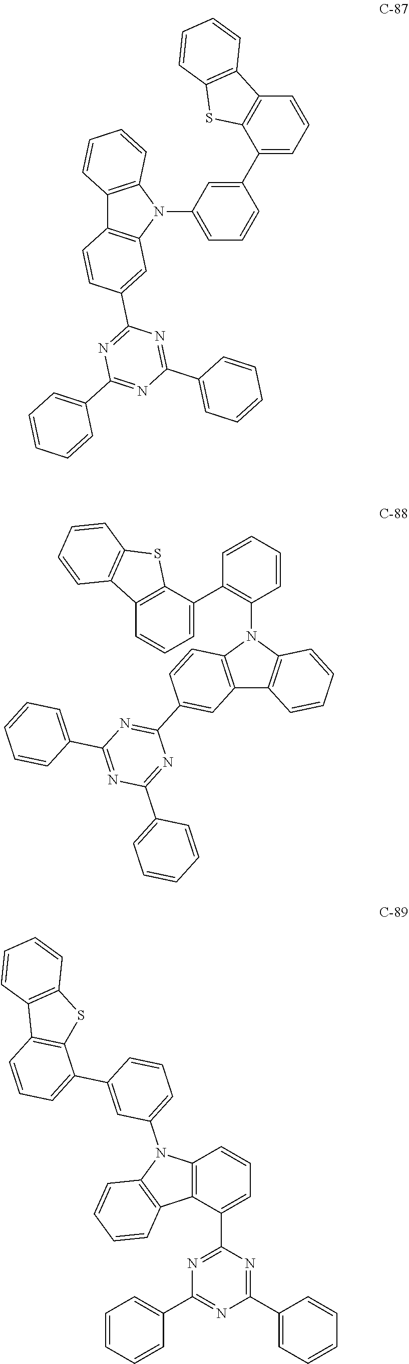 [SMILES: c1ccc(-c2nc(-c3ccccc3)nc(-c3ccc4c5ccccc5n(-c5cccc(-c6cccc7c6sc6ccccc67)c5)c4c3)n2)cc1 ; c1ccc(-c2nc(-c3ccccc3)nc(-c3ccc4c(c3)c3ccccc3n4-c3ccccc3-c3cccc4c3sc3ccccc34)n2)cc1 ; c1ccc(-c2nc(-c3ccccc3)nc(-c3cccc4c3c3ccccc3n4-c3cccc(-c4cccc5c4sc4ccccc45)c3)n2)cc1]

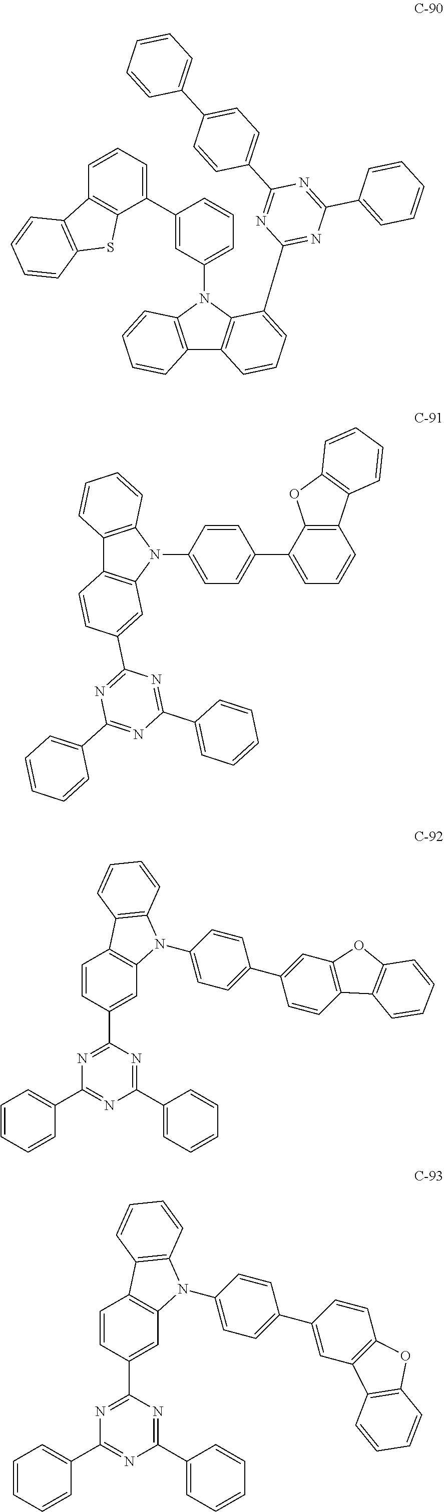 [SMILES: c1ccc(-c2ccc(-c3nc(-c4ccccc4)nc(-c4cccc5c6ccccc6n(-c6cccc(-c7cccc8c7sc7ccccc78)c6)c45)n3)cc2)cc1 ; c1ccc(-c2nc(-c3ccccc3)nc(-c3ccc4c5ccccc5n(-c5ccc(-c6cccc7c6oc6ccccc67)cc5)c4c3)n2)cc1 ; c1ccc(-c2nc(-c3ccccc3)nc(-c3ccc4c5ccccc5n(-c5ccc(-c6ccc7c(c6)oc6ccccc67)cc5)c4c3)n2)cc1 ; c1ccc(-c2nc(-c3ccccc3)nc(-c3ccc4c5ccccc5n(-c5ccc(-c6ccc7oc8ccccc8c7c6)cc5)c4c3)n2)cc1]

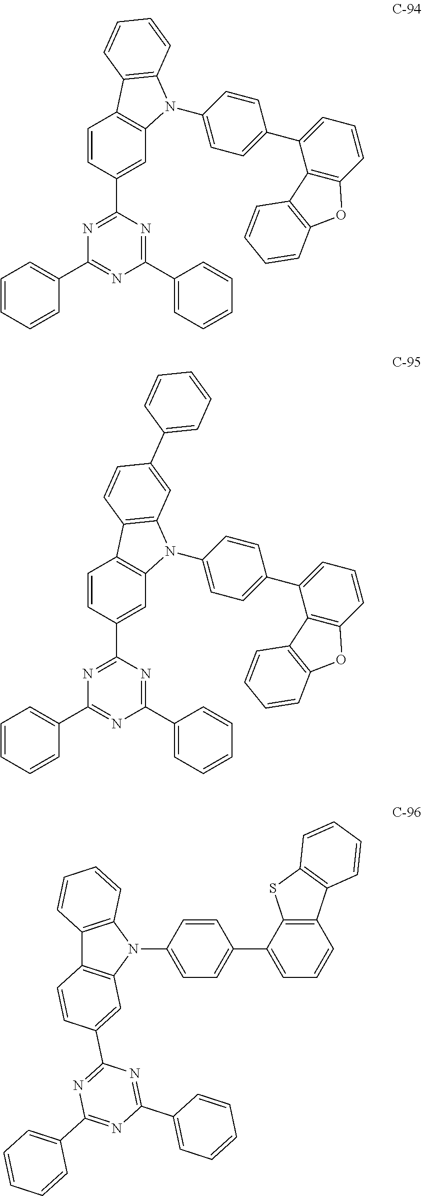

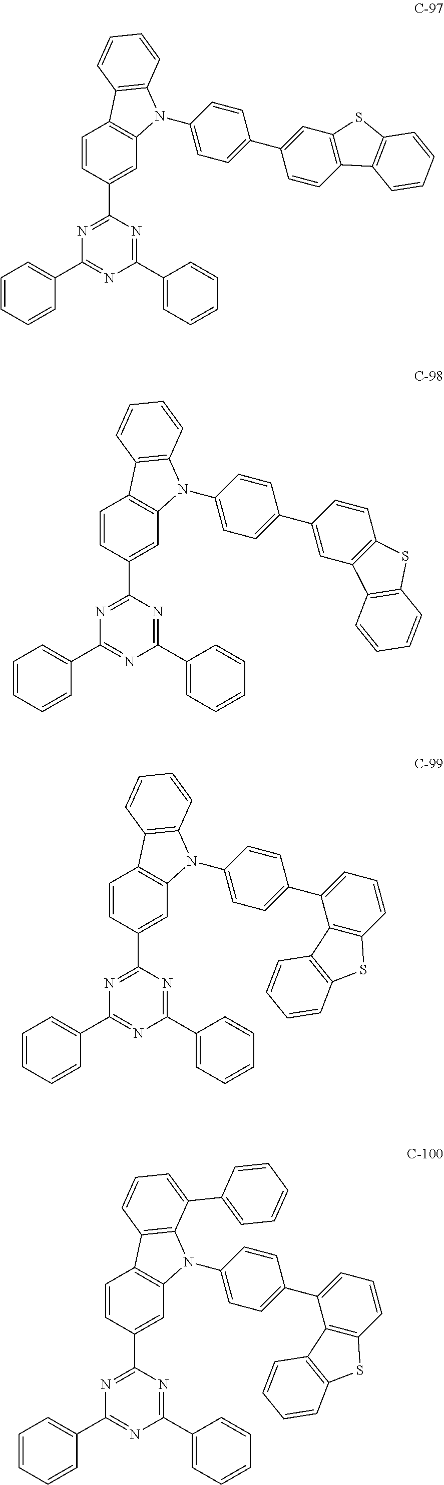 [SMILES: c1ccc(-c2nc(-c3ccccc3)nc(-c3ccc4c5ccccc5n(-c5ccc(-c6ccc7c(c6)sc6ccccc67)cc5)c4c3)n2)cc1 ; c1ccc(-c2nc(-c3ccccc3)nc(-c3ccc4c5ccccc5n(-c5ccc(-c6ccc7sc8ccccc8c7c6)cc5)c4c3)n2)cc1 ; c1ccc(-c2nc(-c3ccccc3)nc(-c3ccc4c5ccccc5n(-c5ccc(-c6cccc7sc8ccccc8c67)cc5)c4c3)n2)cc1 ; c1ccc(-c2nc(-c3ccccc3)nc(-c3ccc4c5cccc(-c6ccccc6)c5n(-c5ccc(-c6cccc7sc8ccccc8c67)cc5)c4c3)n2)cc1]

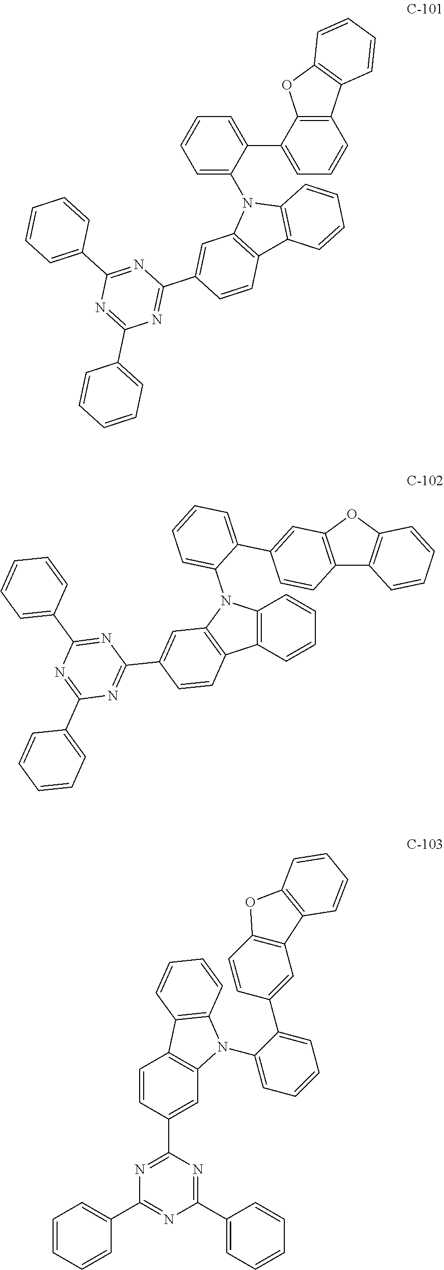 [SMILES: c1ccc(-c2nc(-c3ccccc3)nc(-c3ccc4c5ccccc5n(-c5ccccc5-c5cccc6c5oc5ccccc56)c4c3)n2)cc1 ; c1ccc(-c2nc(-c3ccccc3)nc(-c3ccc4c5ccccc5n(-c5ccccc5-c5ccc6c(c5)oc5ccccc56)c4c3)n2)cc1 ; c1ccc(-c2nc(-c3ccccc3)nc(-c3ccc4c5ccccc5n(-c5ccccc5-c5ccc6oc7ccccc7c6c5)c4c3)n2)cc1]

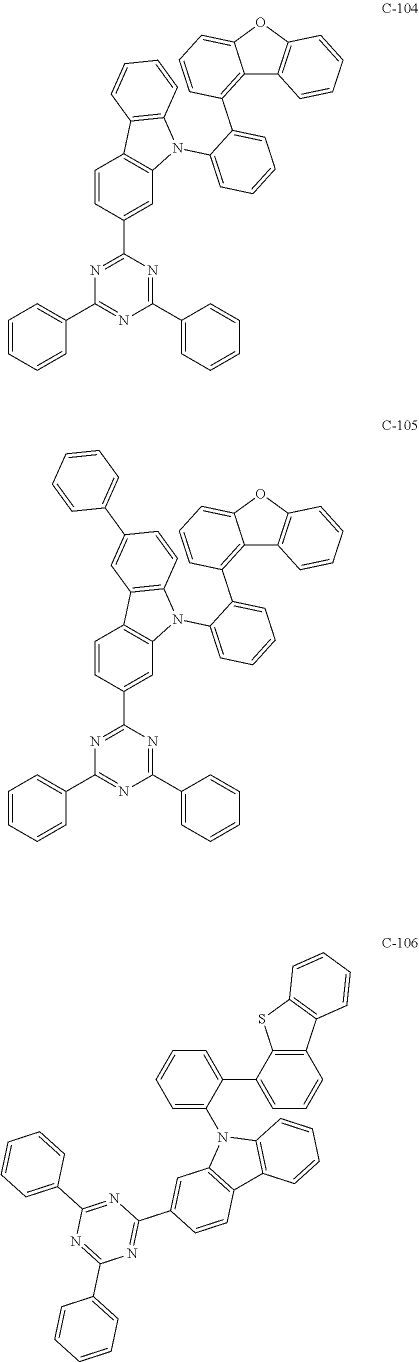 [SMILES: c1ccc(-c2nc(-c3ccccc3)nc(-c3ccc4c5ccccc5n(-c5ccccc5-c5cccc6oc7ccccc7c56)c4c3)n2)cc1 ; c1ccc(-c2ccc3c(c2)c2ccc(-c4nc(-c5ccccc5)nc(-c5ccccc5)n4)cc2n3-c2ccccc2-c2cccc3oc4ccccc4c23)cc1 ; c1ccc(-c2nc(-c3ccccc3)nc(-c3ccc4c5ccccc5n(-c5ccccc5-c5cccc6c5sc5ccccc56)c4c3)n2)cc1]

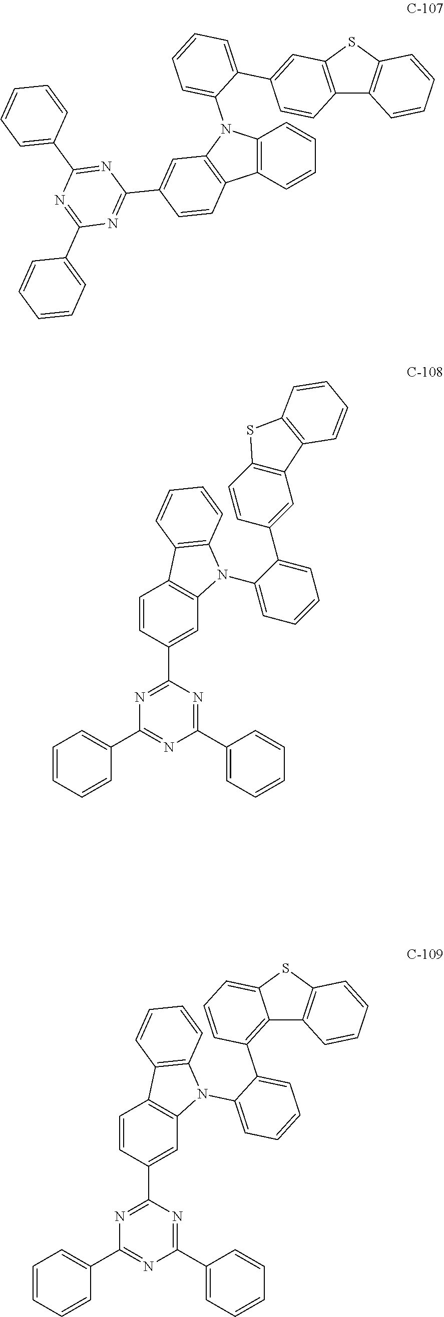 [SMILES: c1ccc(-c2nc(-c3ccccc3)nc(-c3ccc4c5ccccc5n(-c5ccccc5-c5ccc6c(c5)sc5ccccc56)c4c3)n2)cc1 ; c1ccc(-c2nc(-c3ccccc3)nc(-c3ccc4c5ccccc5n(-c5ccccc5-c5ccc6sc7ccccc7c6c5)c4c3)n2)cc1 ; c1ccc(-c2nc(-c3ccccc3)nc(-c3ccc4c5ccccc5n(-c5ccccc5-c5cccc6sc7ccccc7c56)c4c3)n2)cc1]

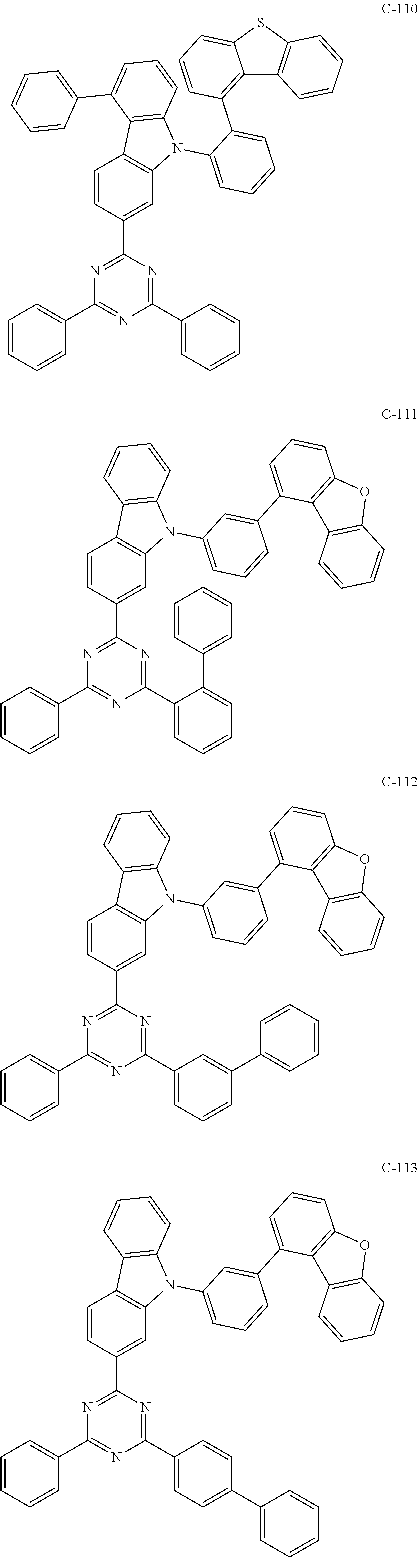 [SMILES: c1ccc(-c2nc(-c3ccccc3)nc(-c3ccc4c5c(-c6ccccc6)cccc5n(-c5ccccc5-c5cccc6sc7ccccc7c56)c4c3)n2)cc1 ; c1ccc(-c2nc(-c3ccc4c5ccccc5n(-c5cccc(-c6cccc7oc8ccccc8c67)c5)c4c3)nc(-c3ccccc3-c3ccccc3)n2)cc1 ; c1ccc(-c2cccc(-c3nc(-c4ccccc4)nc(-c4ccc5c6ccccc6n(-c6cccc(-c7cccc8oc9ccccc9c78)c6)c5c4)n3)c2)cc1 ; c1ccc(-c2ccc(-c3nc(-c4ccccc4)nc(-c4ccc5c6ccccc6n(-c6cccc(-c7cccc8oc9ccccc9c78)c6)c5c4)n3)cc2)cc1]

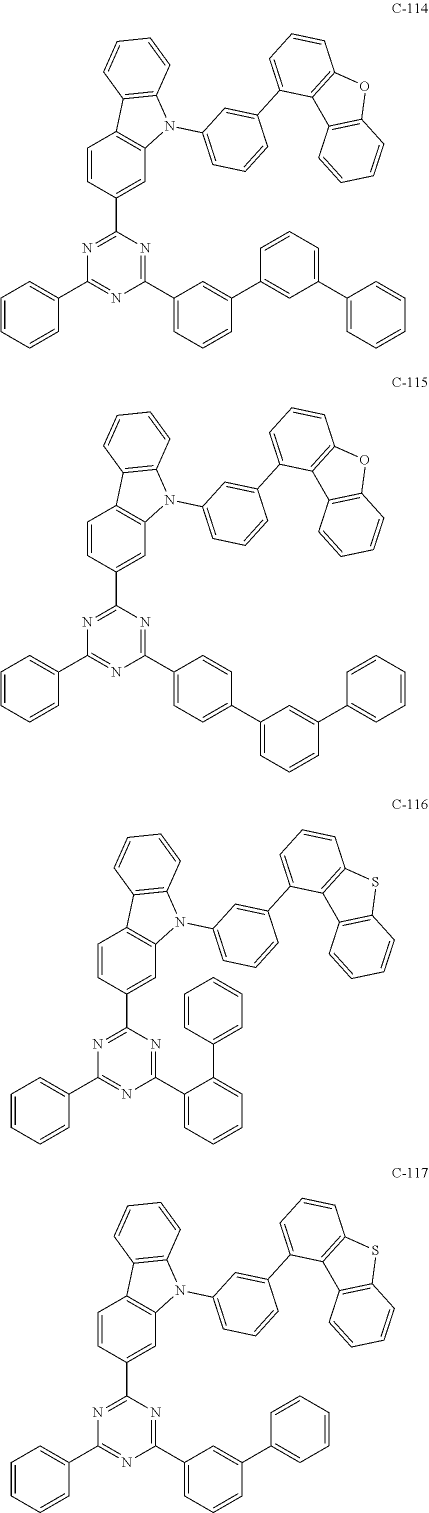 [SMILES: c1ccc(-c2cccc(-c3cccc(-c4nc(-c5ccccc5)nc(-c5ccc6c7ccccc7n(-c7cccc(-c8cccc9oc%10ccccc%10c89)c7)c6c5)n4)c3)c2)cc1 ; c1ccc(-c2cccc(-c3ccc(-c4nc(-c5ccccc5)nc(-c5ccc6c7ccccc7n(-c7cccc(-c8cccc9oc%10ccccc%10c89)c7)c6c5)n4)cc3)c2)cc1 ; c1ccc(-c2nc(-c3ccc4c5ccccc5n(-c5cccc(-c6cccc7sc8ccccc8c67)c5)c4c3)nc(-c3ccccc3-c3ccccc3)n2)cc1 ; c1ccc(-c2cccc(-c3nc(-c4ccccc4)nc(-c4ccc5c6ccccc6n(-c6cccc(-c7cccc8sc9ccccc9c78)c6)c5c4)n3)c2)cc1]

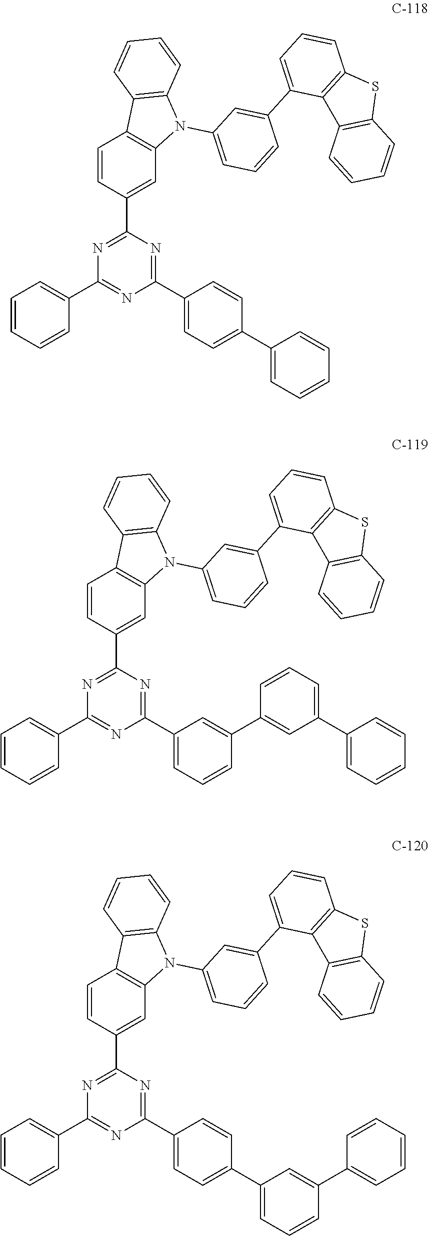 [SMILES: c1ccc(-c2ccc(-c3nc(-c4ccccc4)nc(-c4ccc5c6ccccc6n(-c6cccc(-c7cccc8sc9ccccc9c78)c6)c5c4)n3)cc2)cc1 ; c1ccc(-c2cccc(-c3cccc(-c4nc(-c5ccccc5)nc(-c5ccc6c7ccccc7n(-c7cccc(-c8cccc9sc%10ccccc%10c89)c7)c6c5)n4)c3)c2)cc1 ; c1ccc(-c2cccc(-c3ccc(-c4nc(-c5ccccc5)nc(-c5ccc6c7ccccc7n(-c7cccc(-c8cccc9sc%10ccccc%10c89)c7)c6c5)n4)cc3)c2)cc1]

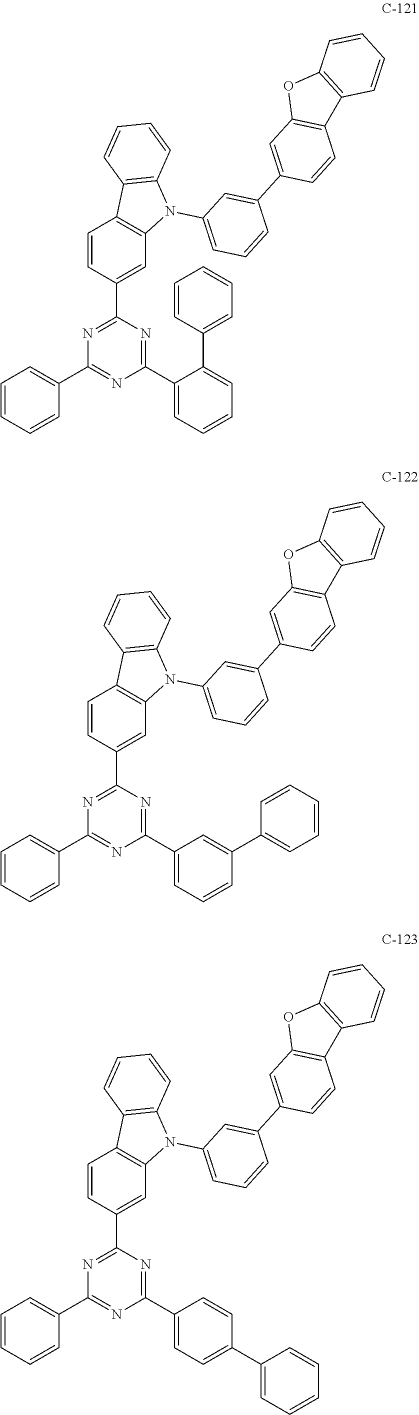 [SMILES: c1ccc(-c2nc(-c3ccc4c5ccccc5n(-c5cccc(-c6ccc7c(c6)oc6ccccc67)c5)c4c3)nc(-c3ccccc3-c3ccccc3)n2)cc1 ; c1ccc(-c2cccc(-c3nc(-c4ccccc4)nc(-c4ccc5c6ccccc6n(-c6cccc(-c7ccc8c(c7)oc7ccccc78)c6)c5c4)n3)c2)cc1 ; c1ccc(-c2ccc(-c3nc(-c4ccccc4)nc(-c4ccc5c6ccccc6n(-c6cccc(-c7ccc8c(c7)oc7ccccc78)c6)c5c4)n3)cc2)cc1]

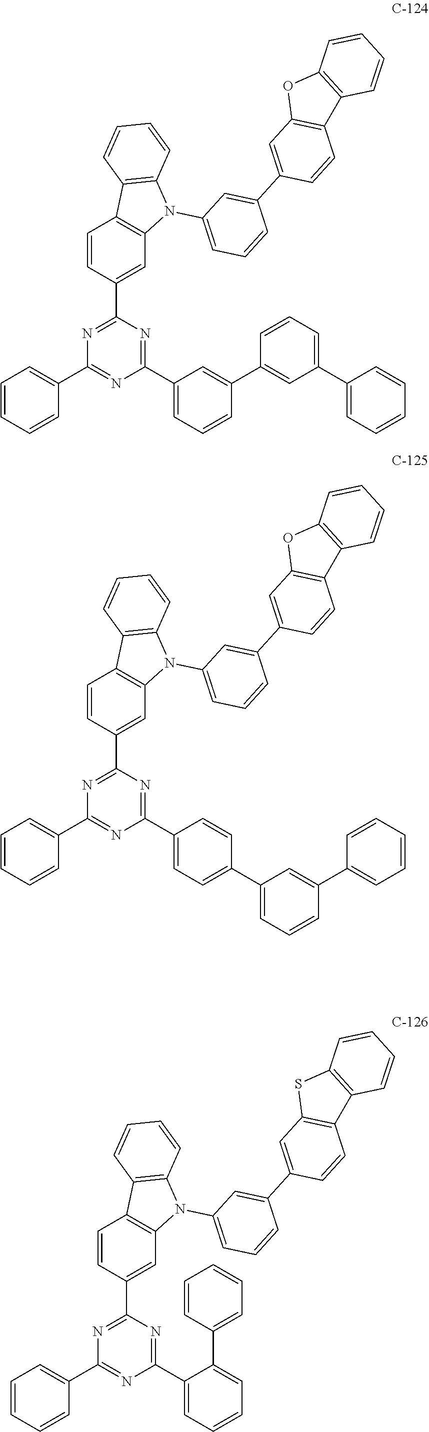 [SMILES: c1ccc(-c2cccc(-c3cccc(-c4nc(-c5ccccc5)nc(-c5ccc6c7ccccc7n(-c7cccc(-c8ccc9c(c8)oc8ccccc89)c7)c6c5)n4)c3)c2)cc1 ; c1ccc(-c2cccc(-c3ccc(-c4nc(-c5ccccc5)nc(-c5ccc6c7ccccc7n(-c7cccc(-c8ccc9c(c8)oc8ccccc89)c7)c6c5)n4)cc3)c2)cc1 ; c1ccc(-c2nc(-c3ccc4c5ccccc5n(-c5cccc(-c6ccc7c(c6)sc6ccccc67)c5)c4c3)nc(-c3ccccc3-c3ccccc3)n2)cc1]

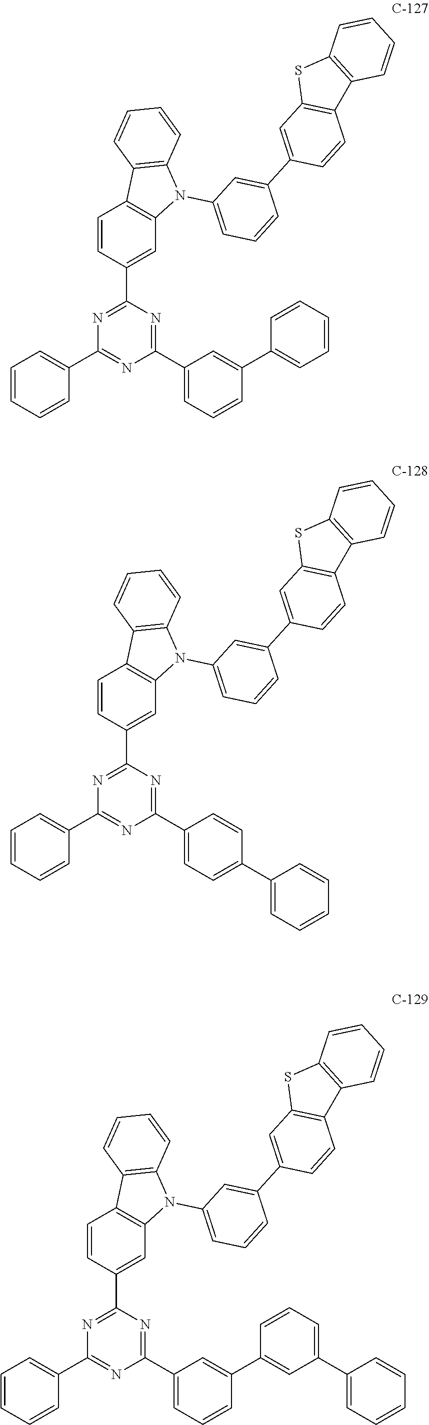 [SMILES: c1ccc(-c2cccc(-c3nc(-c4ccccc4)nc(-c4ccc5c6ccccc6n(-c6cccc(-c7ccc8c(c7)sc7ccccc78)c6)c5c4)n3)c2)cc1 ; c1ccc(-c2ccc(-c3nc(-c4ccccc4)nc(-c4ccc5c6ccccc6n(-c6cccc(-c7ccc8c(c7)sc7ccccc78)c6)c5c4)n3)cc2)cc1 ; c1ccc(-c2cccc(-c3cccc(-c4nc(-c5ccccc5)nc(-c5ccc6c7ccccc7n(-c7cccc(-c8ccc9c(c8)sc8ccccc89)c7)c6c5)n4)c3)c2)cc1]

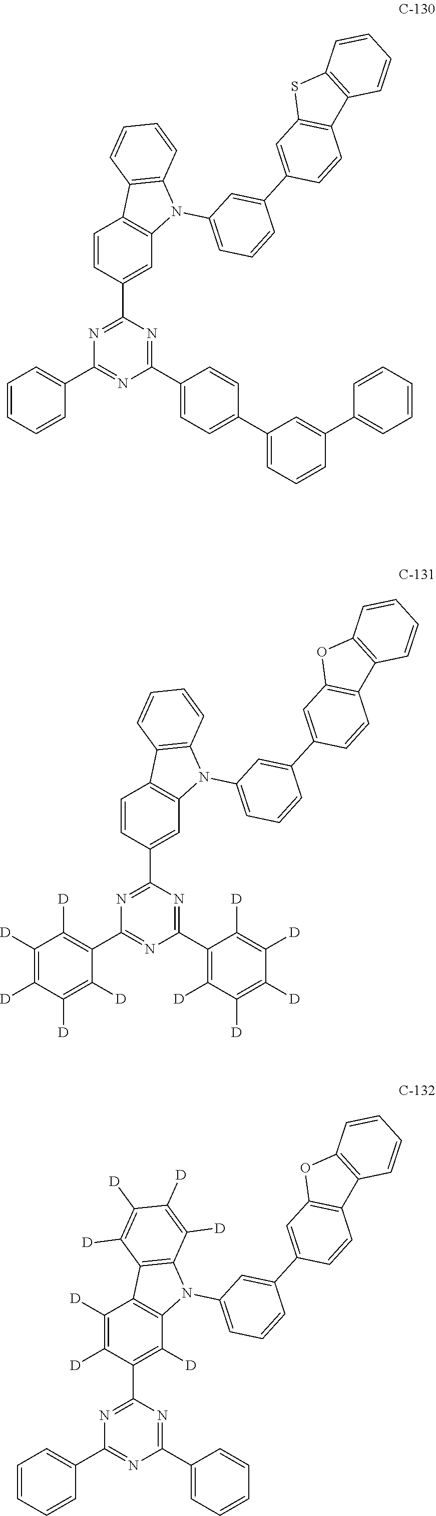 [SMILES: c1ccc(-c2cccc(-c3ccc(-c4nc(-c5ccccc5)nc(-c5ccc6c7ccccc7n(-c7cccc(-c8ccc9c(c8)sc8ccccc89)c7)c6c5)n4)cc3)c2)cc1 ; [2H]c1c([2H])c([2H])c(-c2nc(-c3ccc4c5ccccc5n(-c5cccc(-c6ccc7c(c6)oc6ccccc67)c5)c4c3)nc(-c3c([2H])c([2H])c([2H])c([2H])c3[2H])n2)c([2H])c1[2H] ; [2H]c1c([2H])c([2H])c2c(c1[2H])c1c([2H])c([2H])c(-c3nc(-c4ccccc4)nc(-c4ccccc4)n3)c([2H])c1n2-c1cccc(-c2ccc3c(c2)oc2ccccc23)c1]

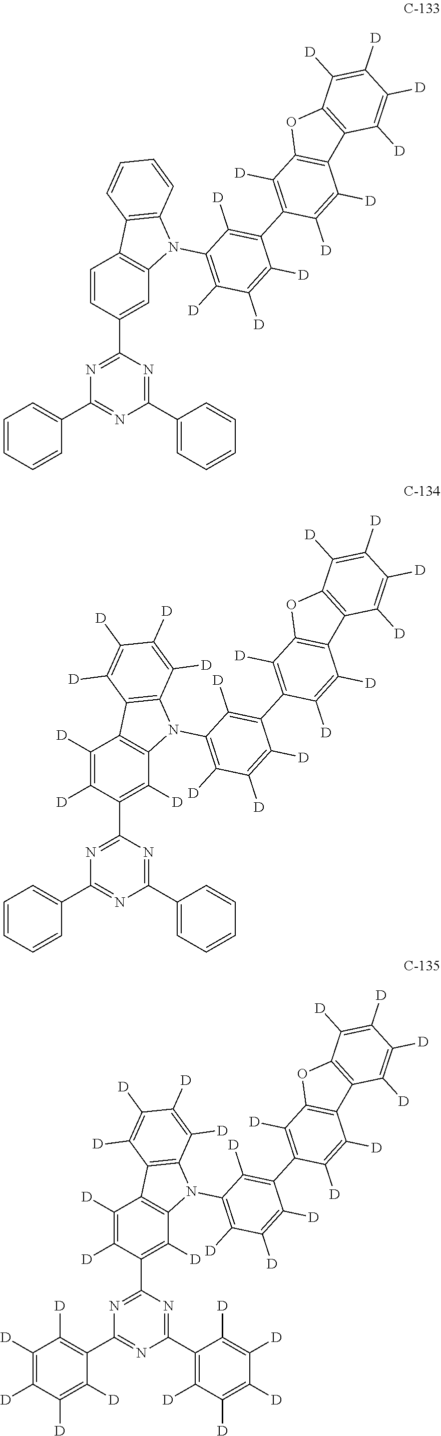

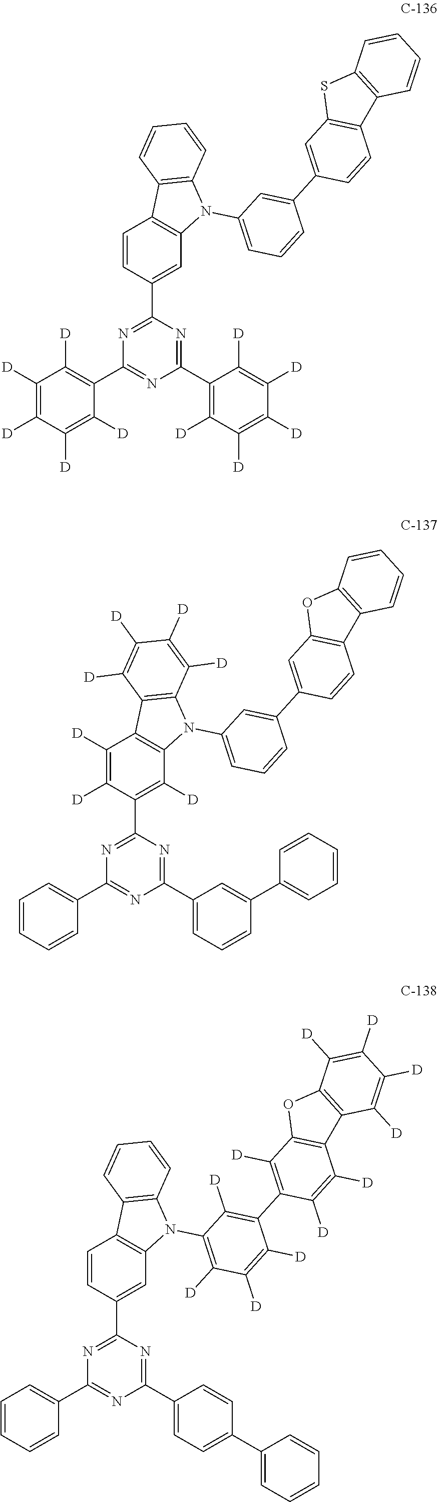 [SMILES: [2H]c1c([2H])c([2H])c(-c2nc(-c3ccc4c5ccccc5n(-c5cccc(-c6ccc7c(c6)sc6ccccc67)c5)c4c3)nc(-c3c([2H])c([2H])c([2H])c([2H])c3[2H])n2)c([2H])c1[2H] ; [2H]c1c([2H])c([2H])c2c(c1[2H])c1c([2H])c([2H])c(-c3nc(-c4ccccc4)nc(-c4cccc(-c5ccccc5)c4)n3)c([2H])c1n2-c1cccc(-c2ccc3c(c2)oc2ccccc23)c1 ; [2H]c1c([2H])c(-c2c([2H])c([2H])c3c(oc4c([2H])c([2H])c([2H])c([2H])c43)c2[2H])c([2H])c(-n2c3ccccc3c3ccc(-c4nc(-c5ccccc5)nc(-c5ccc(-c6ccccc6)cc5)n4)cc32)c1[2H]]

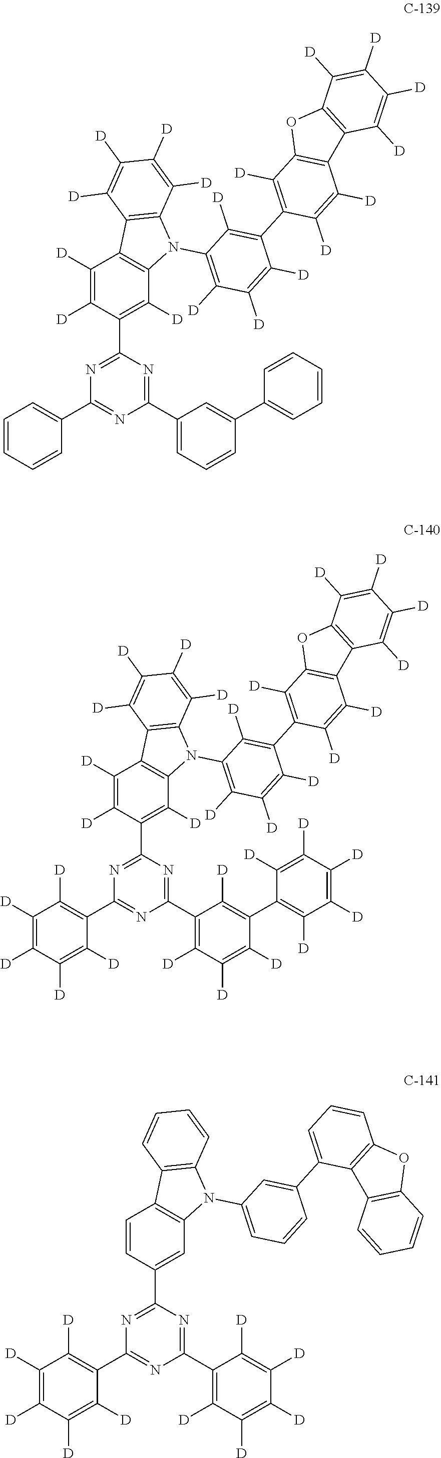

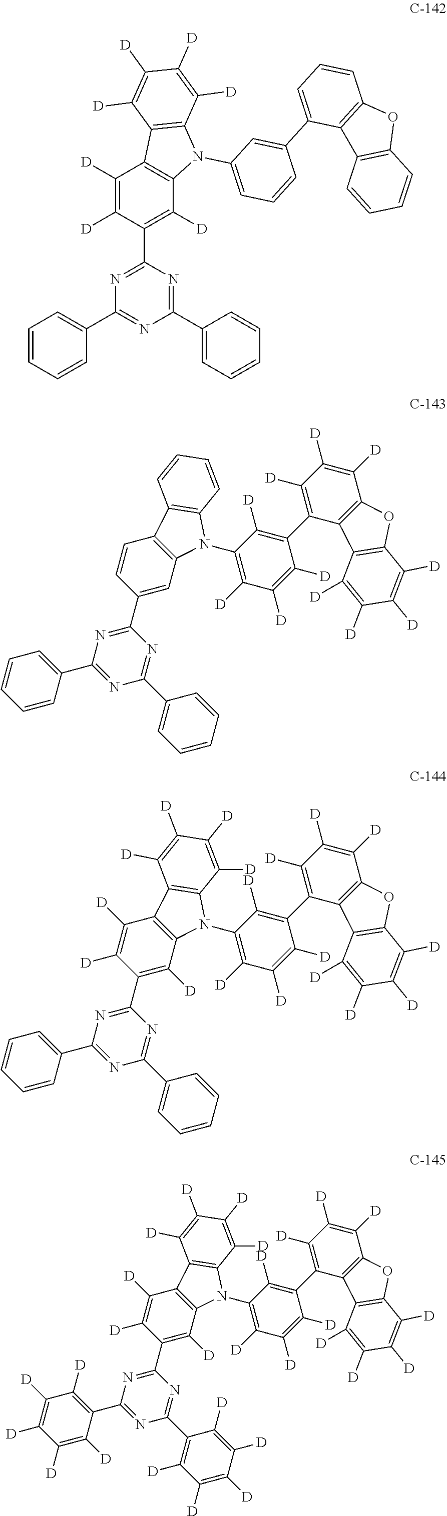

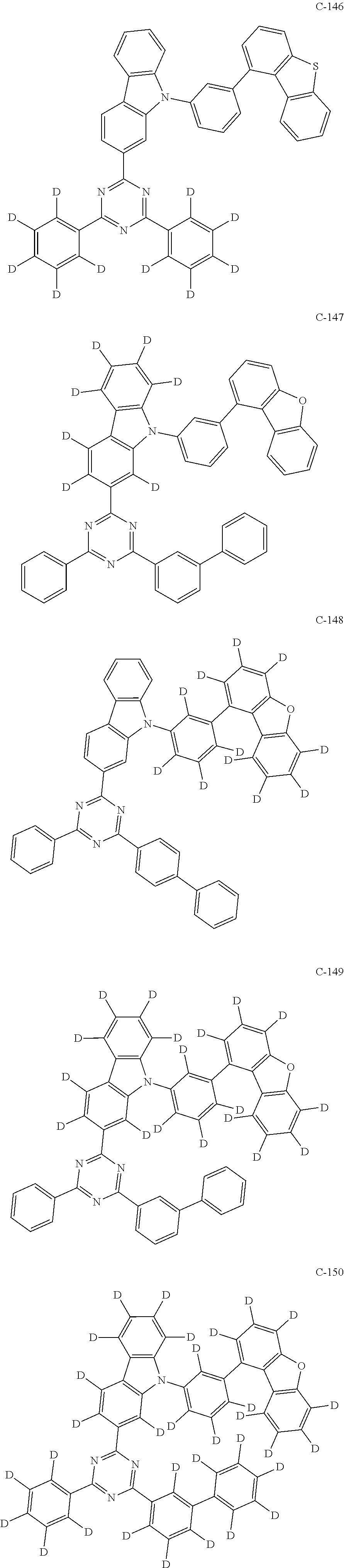

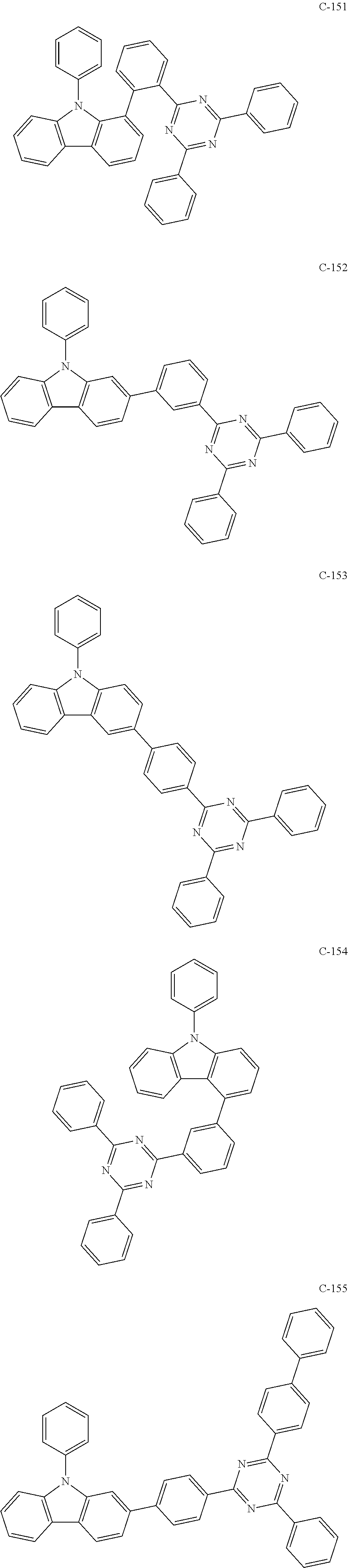

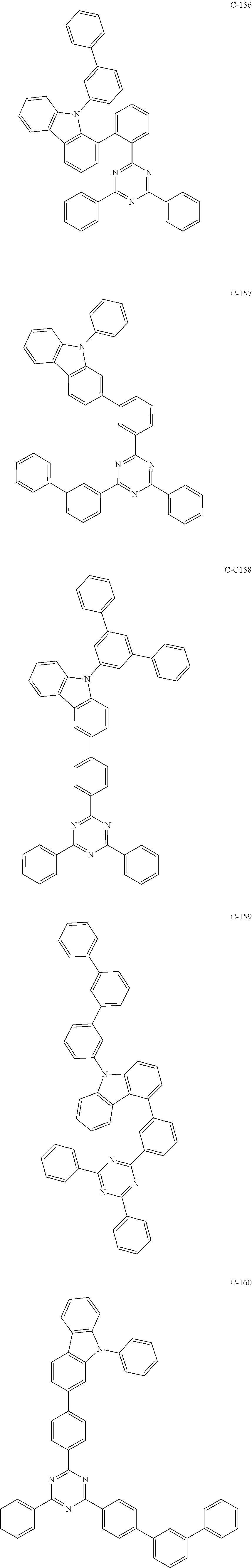 [SMILES: c1ccc(-c2cccc(-n3c4ccccc4c4cccc(-c5ccccc5-c5nc(-c6ccccc6)nc(-c6ccccc6)n5)c43)c2)cc1 ; c1ccc(-c2cccc(-c3nc(-c4ccccc4)nc(-c4cccc(-c5ccc6c7ccccc7n(-c7ccccc7)c6c5)c4)n3)c2)cc1 ; c1ccc(-c2cc(-c3ccccc3)cc(-n3c4ccccc4c4cc(-c5ccc(-c6nc(-c7ccccc7)nc(-c7ccccc7)n6)cc5)ccc43)c2)cc1 ; c1ccc(-c2cccc(-c3cccc(-n4c5ccccc5c5c(-c6cccc(-c7nc(-c8ccccc8)nc(-c8ccccc8)n7)c6)cccc54)c3)c2)cc1 ; c1ccc(-c2cccc(-c3ccc(-c4nc(-c5ccccc5)nc(-c5ccc(-c6ccc7c8ccccc8n(-c8ccccc8)c7c6)cc5)n4)cc3)c2)cc1]

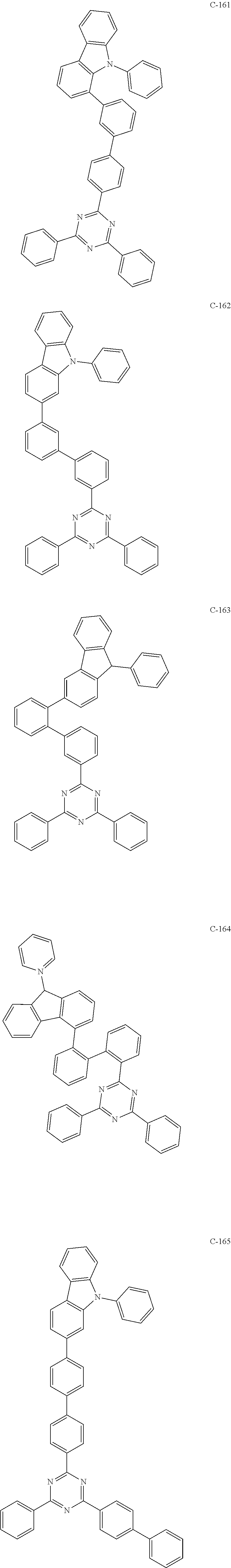

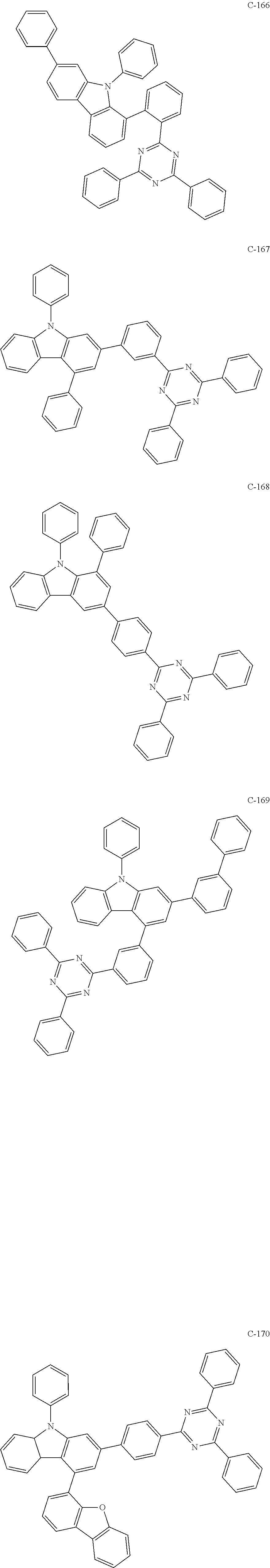 [SMILES: c1ccc(-c2ccc3c4cccc(-c5ccccc5-c5nc(-c6ccccc6)nc(-c6ccccc6)n5)c4n(-c4ccccc4)c3c2)cc1 ; c1ccc(-c2nc(-c3ccccc3)nc(-c3cccc(-c4cc(-c5ccccc5)c5c6ccccc6n(-c6ccccc6)c5c4)c3)n2)cc1 ; c1ccc(-c2nc(-c3ccccc3)nc(-c3ccc(-c4cc(-c5ccccc5)c5c(c4)c4ccccc4n5-c4ccccc4)cc3)n2)cc1 ; c1ccc(-c2cccc(-c3cc(-c4cccc(-c5nc(-c6ccccc6)nc(-c6ccccc6)n5)c4)c4c5ccccc5n(-c5ccccc5)c4c3)c2)cc1 ; C1=CC2c3c(-c4cccc5c4oc4ccccc45)cc(-c4ccc(-c5nc(-c6ccccc6)nc(-c6ccccc6)n5)cc4)cc3N(c3ccccc3)C2C=C1]

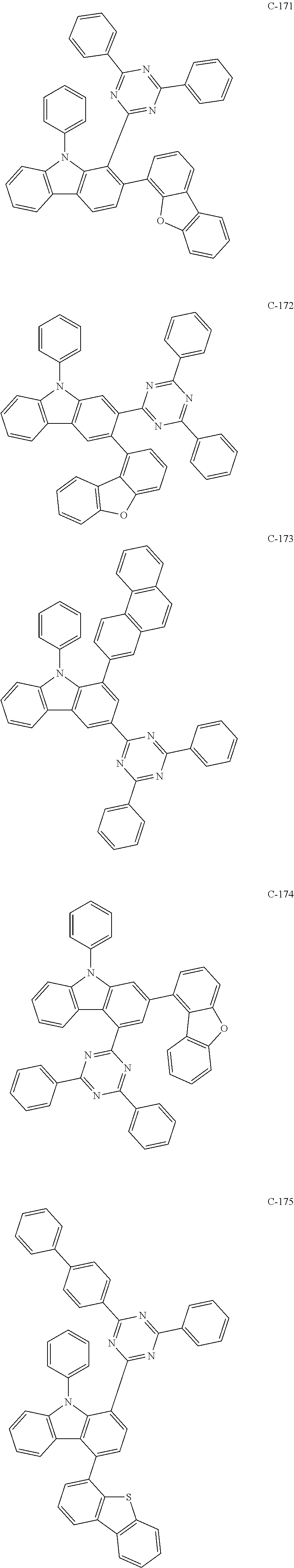 [SMILES: c1ccc(-c2nc(-c3ccccc3)nc(-c3c(-c4cccc5c4oc4ccccc45)ccc4c5ccccc5n(-c5ccccc5)c34)n2)cc1 ; c1ccc(-c2nc(-c3ccccc3)nc(-c3cc4c(cc3-c3cccc5oc6ccccc6c35)c3ccccc3n4-c3ccccc3)n2)cc1 ; c1ccc(-c2nc(-c3ccccc3)nc(-c3cc(-c4ccc5c(ccc6ccccc65)c4)c4c(c3)c3ccccc3n4-c3ccccc3)n2)cc1 ; c1ccc(-c2nc(-c3ccccc3)nc(-c3cc(-c4cccc5oc6ccccc6c45)cc4c3c3ccccc3n4-c3ccccc3)n2)cc1 ; c1ccc(-c2ccc(-c3nc(-c4ccccc4)nc(-c4ccc(-c5cccc6c5sc5ccccc56)c5c6ccccc6n(-c6ccccc6)c45)n3)cc2)cc1]

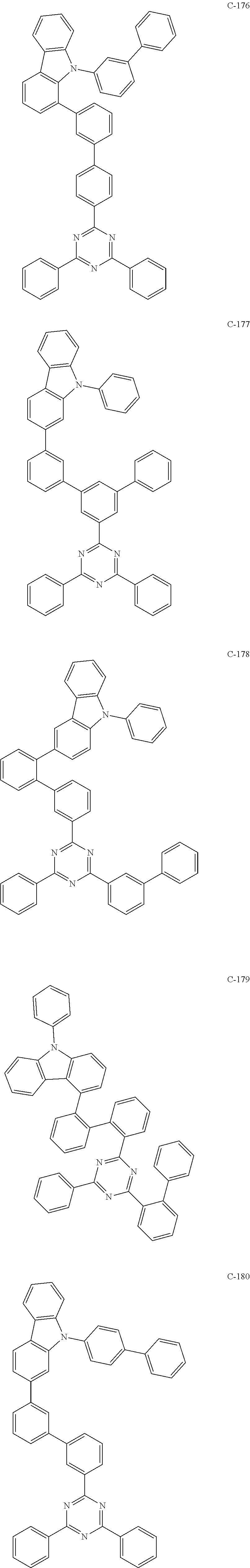 [SMILES: c1ccc(-c2cccc(-n3c4ccccc4c4cccc(-c5cccc(-c6ccc(-c7nc(-c8ccccc8)nc(-c8ccccc8)n7)cc6)c5)c43)c2)cc1 ; c1ccc(-c2cc(-c3cccc(-c4ccc5c6ccccc6n(-c6ccccc6)c5c4)c3)cc(-c3nc(-c4ccccc4)nc(-c4ccccc4)n3)c2)cc1 ; c1ccc(-c2cccc(-c3nc(-c4ccccc4)nc(-c4cccc(-c5ccccc5-c5ccc6c(c5)c5ccccc5n6-c5ccccc5)c4)n3)c2)cc1 ; c1ccc(-c2nc(-c3ccccc3-c3ccccc3)nc(-c3ccccc3-c3ccccc3-c3cccc4c3c3ccccc3n4-c3ccccc3)n2)cc1 ; c1ccc(-c2ccc(-n3c4ccccc4c4ccc(-c5cccc(-c6cccc(-c7nc(-c8ccccc8)nc(-c8ccccc8)n7)c6)c5)cc43)cc2)cc1]

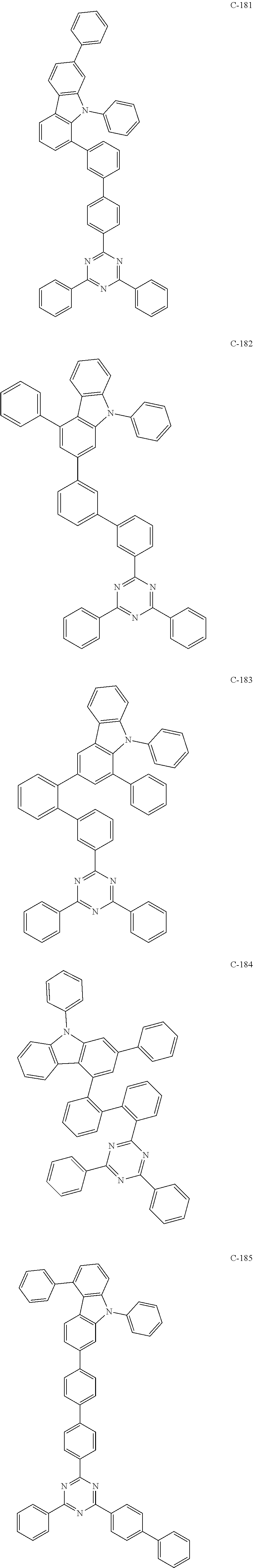 [SMILES: c1ccc(-c2ccc3c4cccc(-c5cccc(-c6ccc(-c7nc(-c8ccccc8)nc(-c8ccccc8)n7)cc6)c5)c4n(-c4ccccc4)c3c2)cc1 ; c1ccc(-c2nc(-c3ccccc3)nc(-c3cccc(-c4cccc(-c5cc(-c6ccccc6)c6c7ccccc7n(-c7ccccc7)c6c5)c4)c3)n2)cc1 ; c1ccc(-c2nc(-c3ccccc3)nc(-c3cccc(-c4ccccc4-c4cc(-c5ccccc5)c5c(c4)c4ccccc4n5-c4ccccc4)c3)n2)cc1 ; c1ccc(-c2cc(-c3ccccc3-c3ccccc3-c3nc(-c4ccccc4)nc(-c4ccccc4)n3)c3c4ccccc4n(-c4ccccc4)c3c2)cc1 ; c1ccc(-c2ccc(-c3nc(-c4ccccc4)nc(-c4ccc(-c5ccc(-c6ccc7c8c(-c9ccccc9)cccc8n(-c8ccccc8)c7c6)cc5)cc4)n3)cc2)cc1]

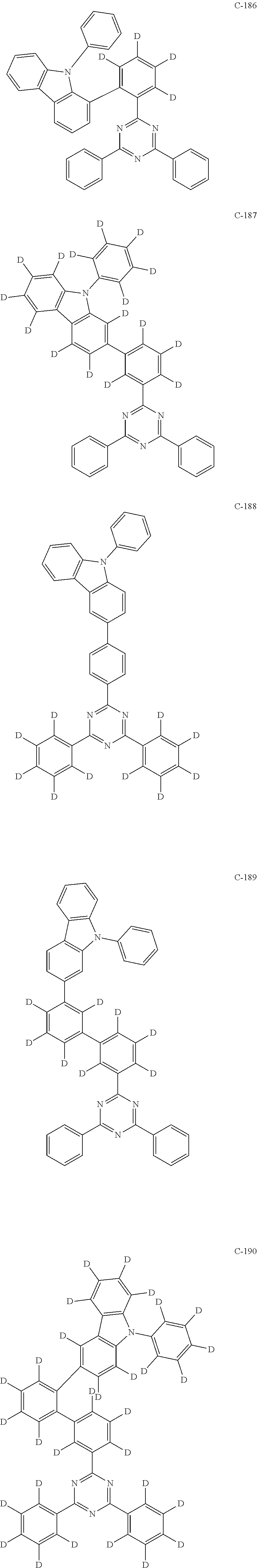

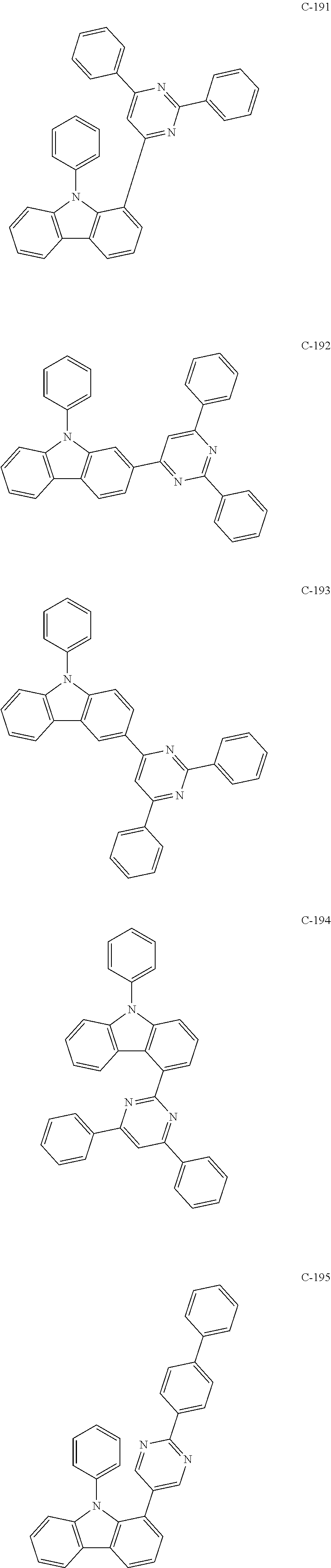 [SMILES: c1ccc(-c2cc(-c3cccc4c5ccccc5n(-c5ccccc5)c34)nc(-c3ccccc3)n2)cc1 ; c1ccc(-c2cc(-c3ccc4c5ccccc5n(-c5ccccc5)c4c3)nc(-c3ccccc3)n2)cc1 ; c1ccc(-c2cc(-c3ccc4c(c3)c3ccccc3n4-c3ccccc3)nc(-c3ccccc3)n2)cc1 ; c1ccc(-c2cc(-c3ccccc3)nc(-c3cccc4c3c3ccccc3n4-c3ccccc3)n2)cc1 ; c1ccc(-c2ccc(-c3ncc(-c4cccc5c6ccccc6n(-c6ccccc6)c45)cn3)cc2)cc1]

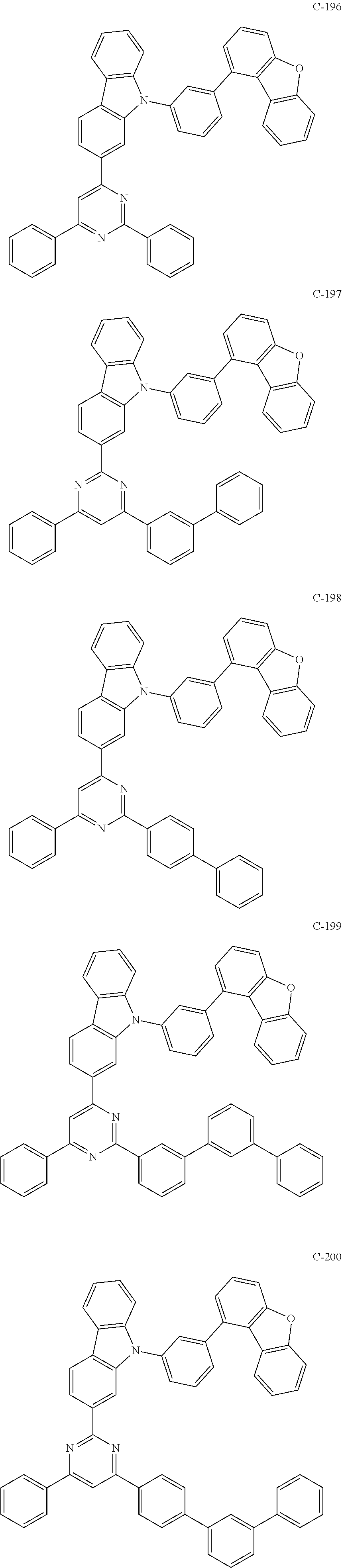

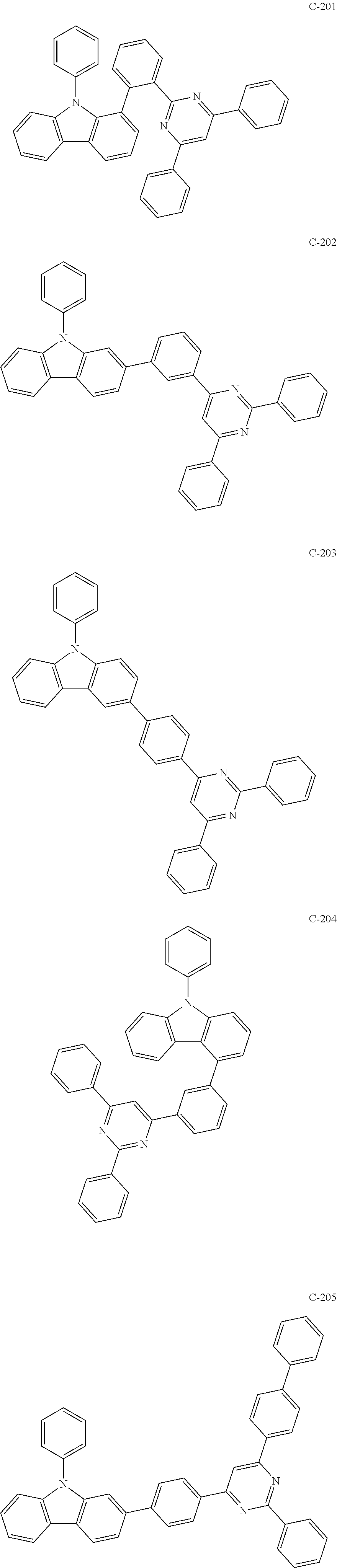

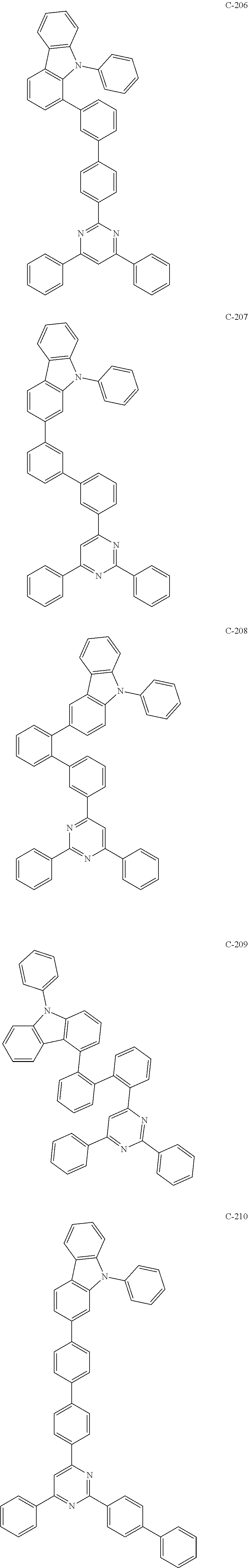 [SMILES: c1ccc(-c2cc(-c3ccccc3)nc(-c3ccc(-c4cccc(-c5cccc6c7ccccc7n(-c7ccccc7)c56)c4)cc3)n2)cc1 ; c1ccc(-c2cc(-c3cccc(-c4cccc(-c5ccc6c7ccccc7n(-c7ccccc7)c6c5)c4)c3)nc(-c3ccccc3)n2)cc1 ; c1ccc(-c2cc(-c3cccc(-c4ccccc4-c4ccc5c(c4)c4ccccc4n5-c4ccccc4)c3)nc(-c3ccccc3)n2)cc1 ; c1ccc(-c2cc(-c3ccccc3-c3ccccc3-c3cccc4c3c3ccccc3n4-c3ccccc3)nc(-c3ccccc3)n2)cc1 ; c1ccc(-c2ccc(-c3nc(-c4ccccc4)cc(-c4ccc(-c5ccc(-c6ccc7c8ccccc8n(-c8ccccc8)c7c6)cc5)cc4)n3)cc2)cc1]

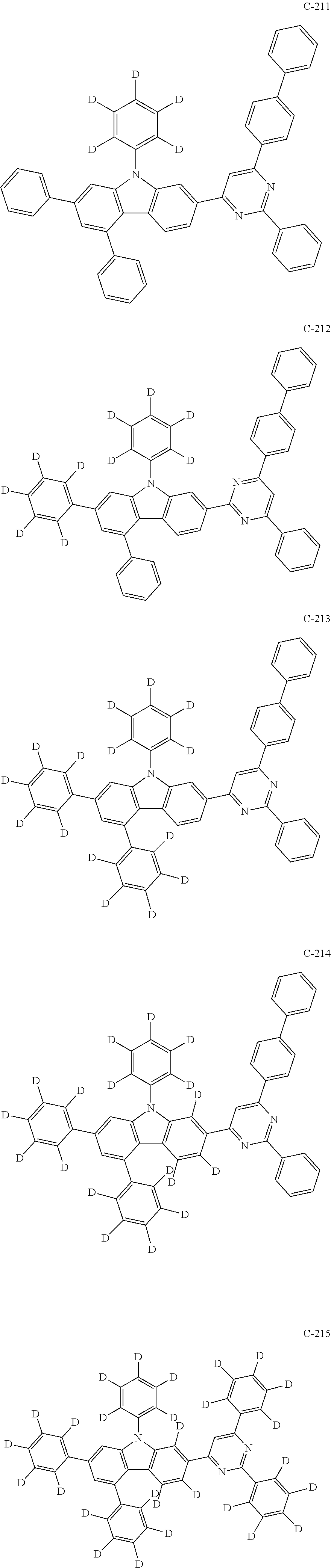

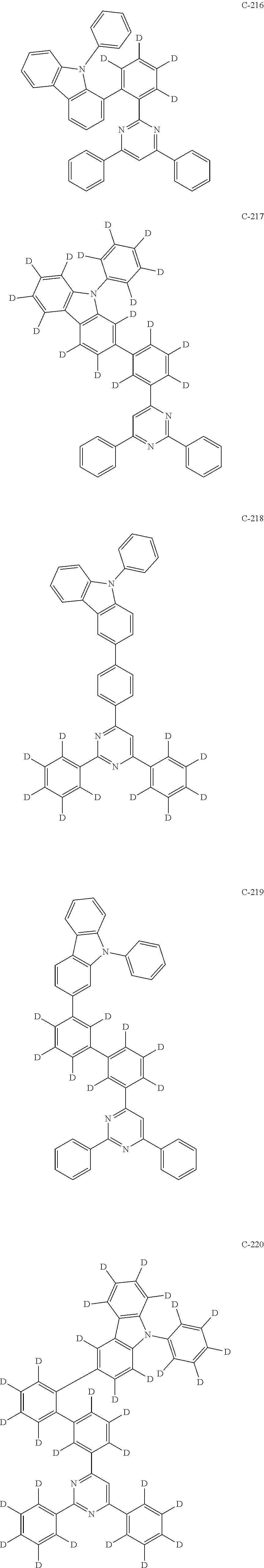

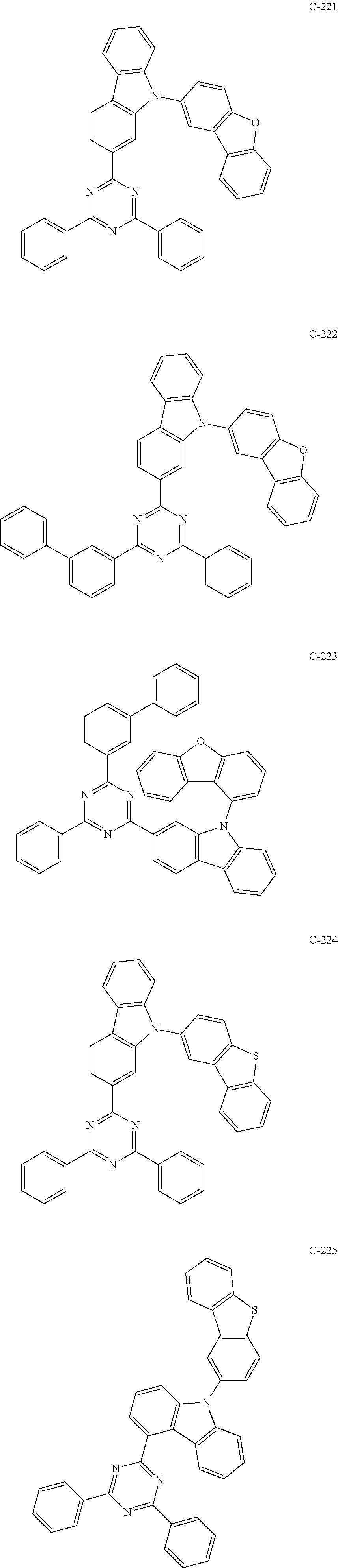 [SMILES: c1ccc(-c2nc(-c3ccccc3)nc(-c3ccc4c5ccccc5n(-c5ccc6oc7ccccc7c6c5)c4c3)n2)cc1 ; c1ccc(-c2cccc(-c3nc(-c4ccccc4)nc(-c4ccc5c6ccccc6n(-c6ccc7oc8ccccc8c7c6)c5c4)n3)c2)cc1 ; c1ccc(-c2cccc(-c3nc(-c4ccccc4)nc(-c4ccc5c6ccccc6n(-c6cccc7oc8ccccc8c67)c5c4)n3)c2)cc1 ; c1ccc(-c2nc(-c3ccccc3)nc(-c3ccc4c5ccccc5n(-c5ccc6sc7ccccc7c6c5)c4c3)n2)cc1 ; c1ccc(-c2nc(-c3ccccc3)nc(-c3cccc4c3c3ccccc3n4-c3ccc4sc5ccccc5c4c3)n2)cc1]

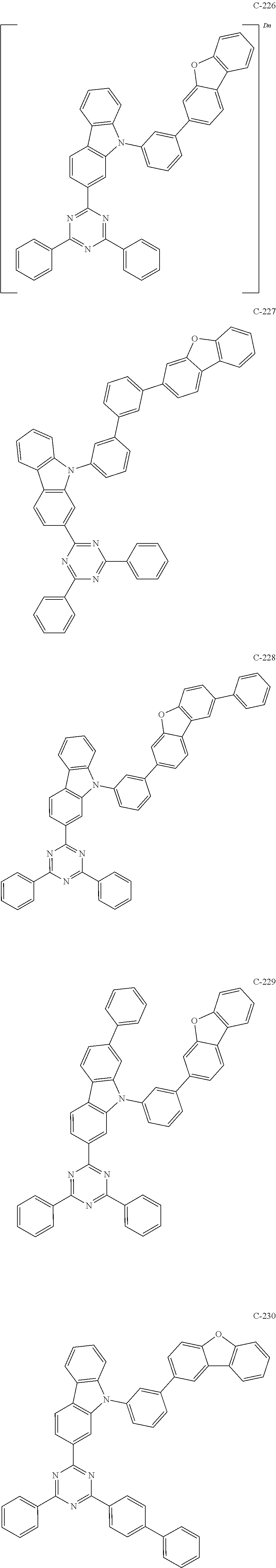

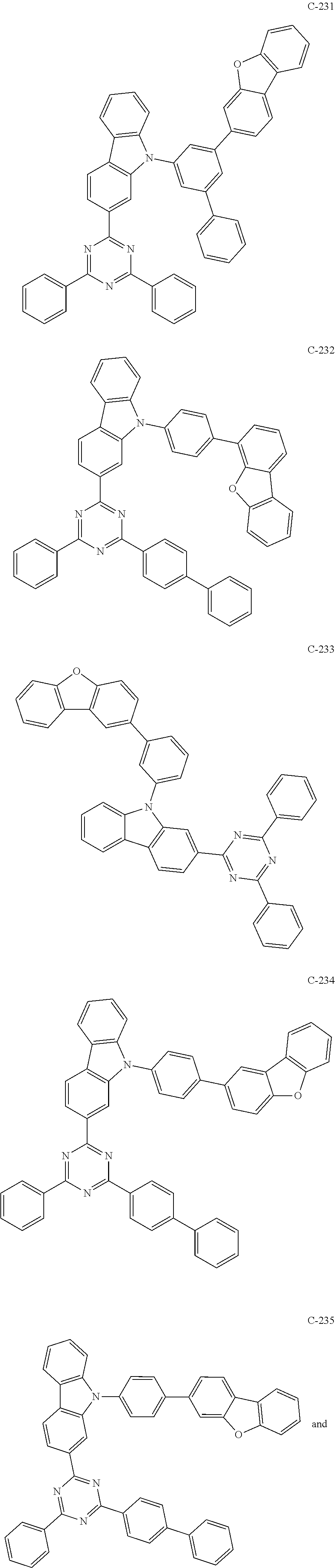 [SMILES: c1ccc(-c2cc(-c3ccc4c(c3)oc3ccccc34)cc(-n3c4ccccc4c4ccc(-c5nc(-c6ccccc6)nc(-c6ccccc6)n5)cc43)c2)cc1 ; c1ccc(-c2ccc(-c3nc(-c4ccccc4)nc(-c4ccc5c6ccccc6n(-c6ccc(-c7cccc8c7oc7ccccc78)cc6)c5c4)n3)cc2)cc1 ; c1ccc(-c2nc(-c3ccccc3)nc(-c3ccc4c5ccccc5n(-c5cccc(-c6ccc7oc8ccccc8c7c6)c5)c4c3)n2)cc1 ; c1ccc(-c2ccc(-c3nc(-c4ccccc4)nc(-c4ccc5c6ccccc6n(-c6ccc(-c7ccc8oc9ccccc9c8c7)cc6)c5c4)n3)cc2)cc1 ; c1ccc(-c2ccc(-c3nc(-c4ccccc4)nc(-c4ccc5c6ccccc6n(-c6ccc(-c7ccc8c(c7)oc7ccccc78)cc6)c5c4)n3)cc2)cc1]

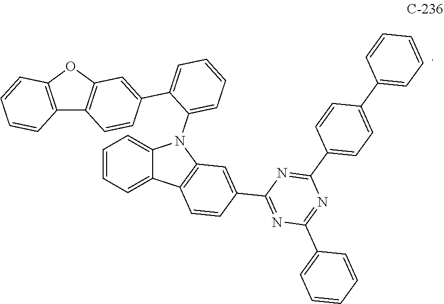 [SMILES: c1ccc(-c2ccc(-c3nc(-c4ccccc4)nc(-c4ccc5c6ccccc6n(-c6ccccc6-c6ccc7c(c6)oc6ccccc67)c5c4)n3)cc2)cc1]

14. The plurality of host materials according to claim 1, wherein the compound represented by Formula 2 is at least one selected from the following compounds: 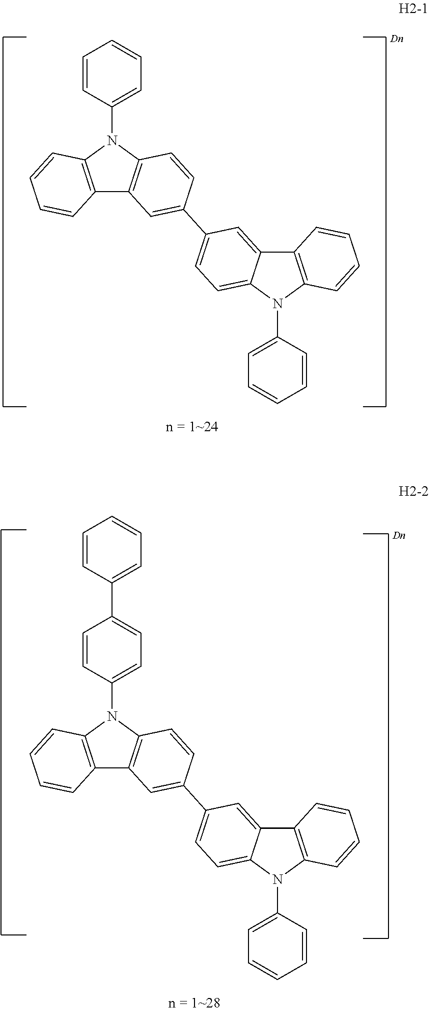 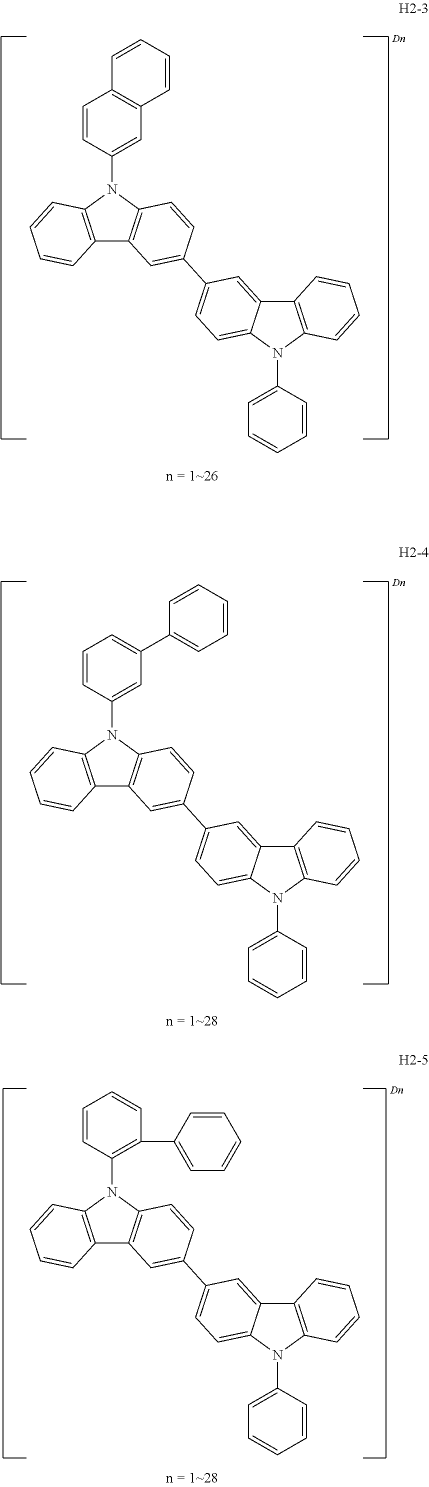 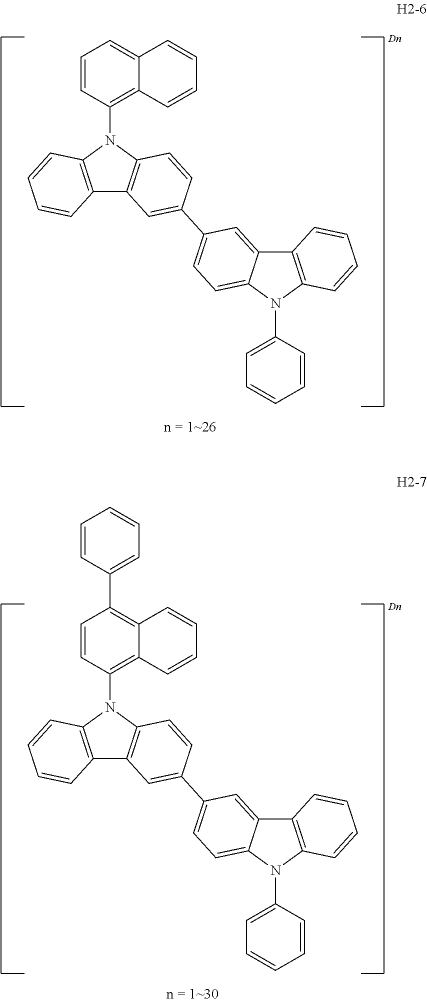 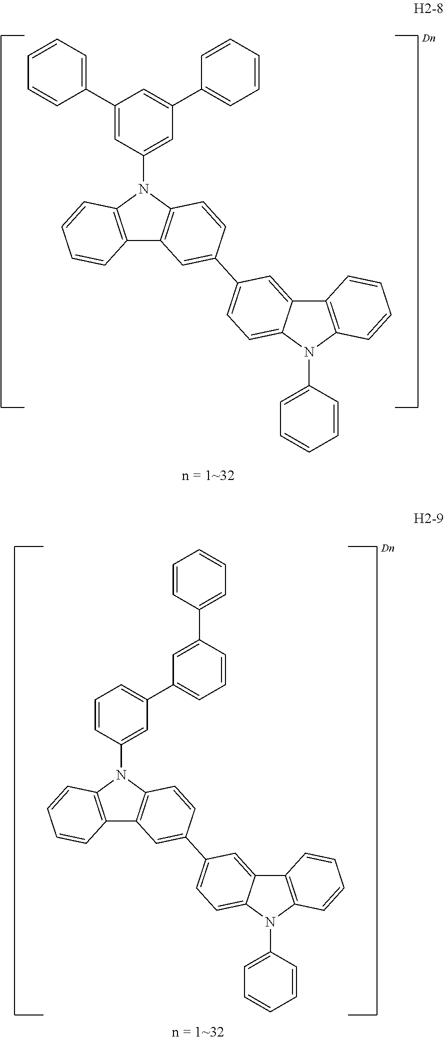 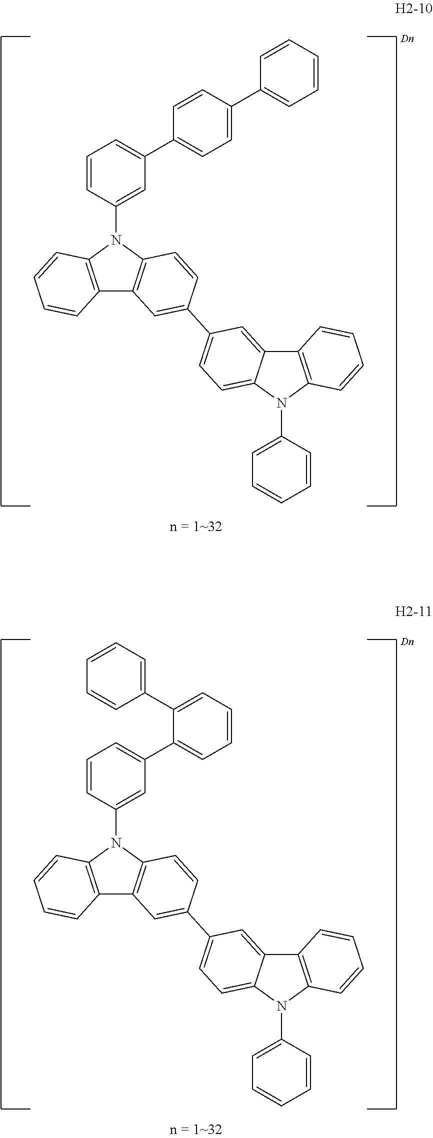 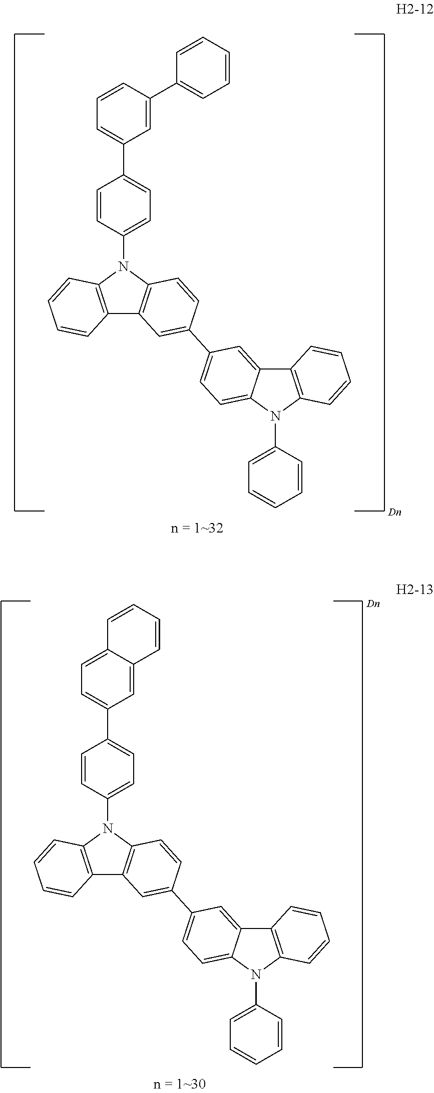 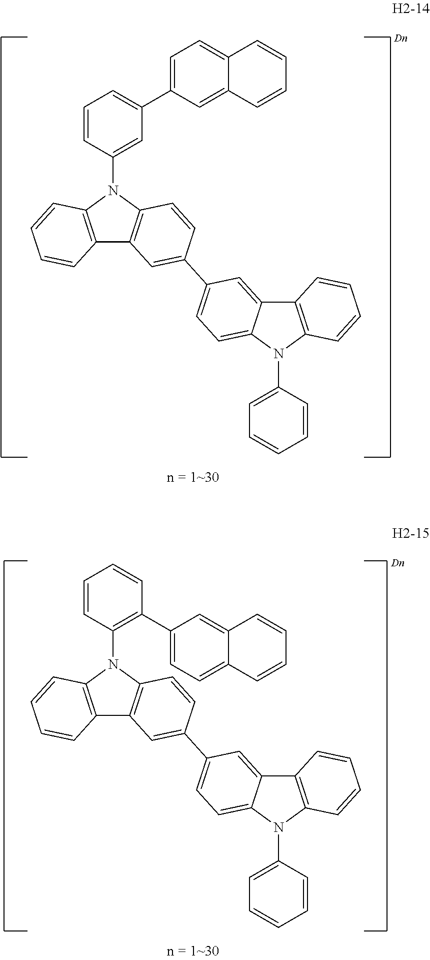 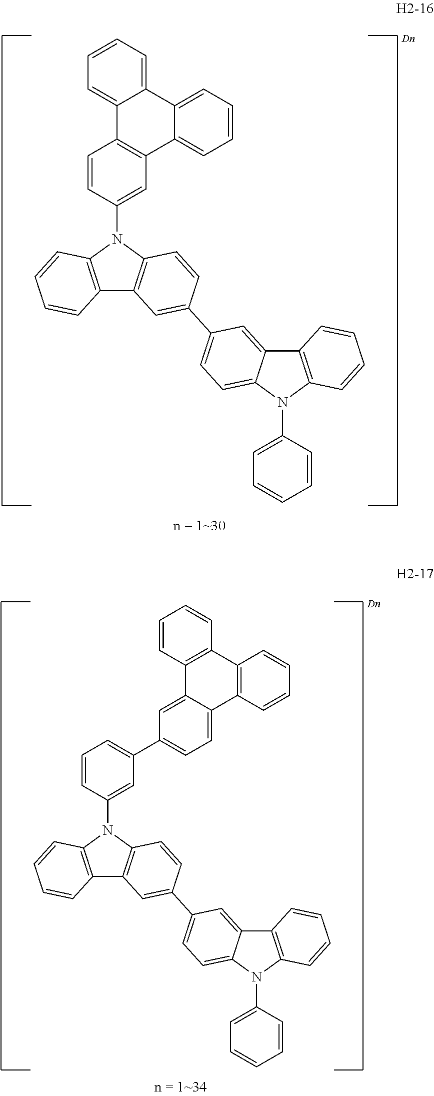 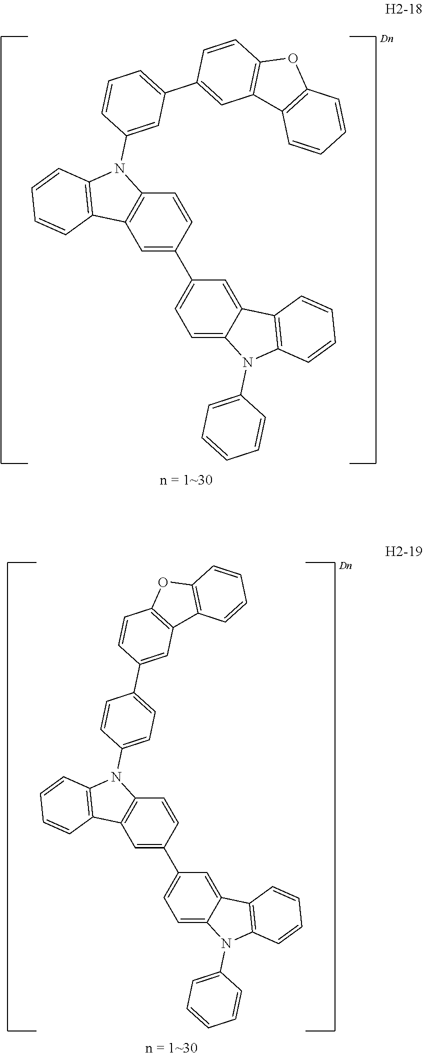 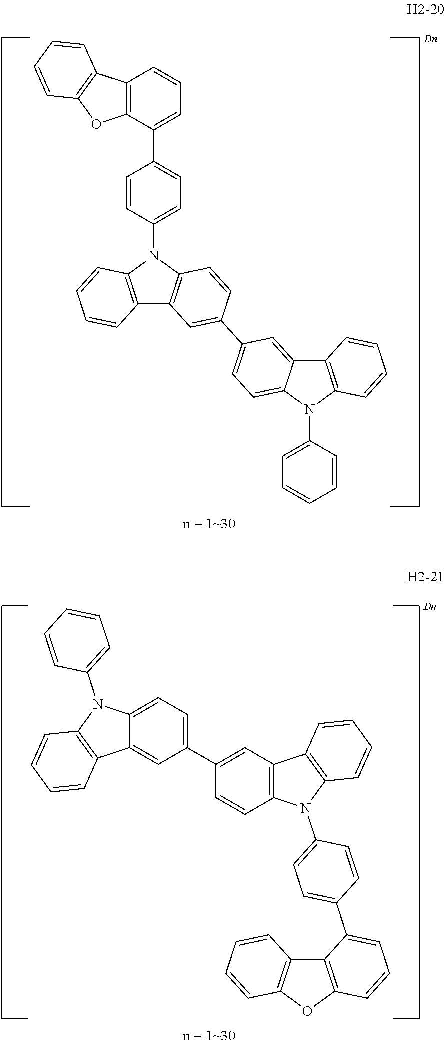 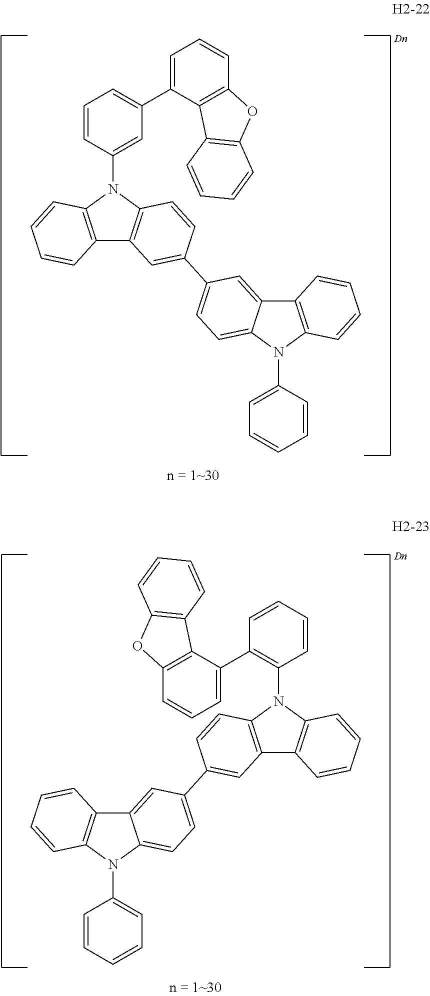 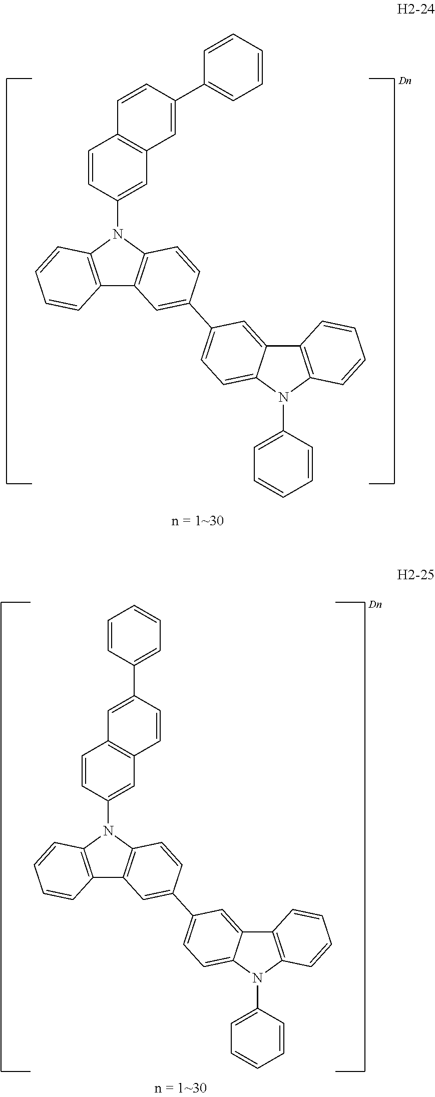 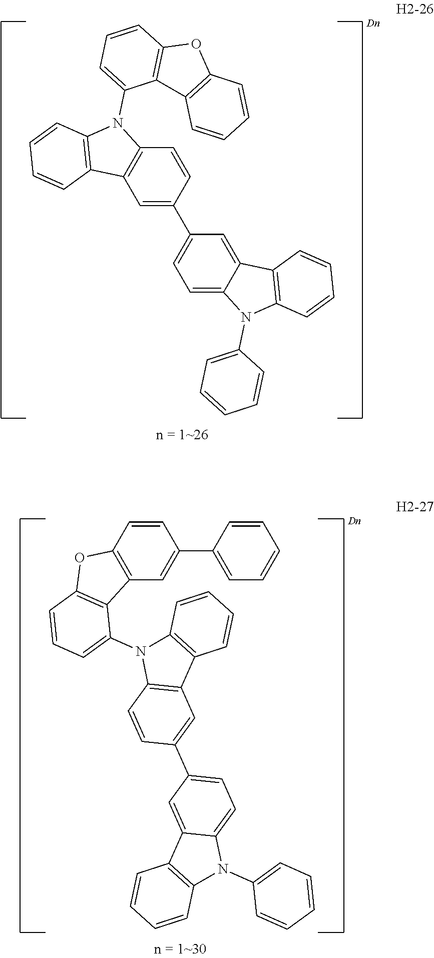 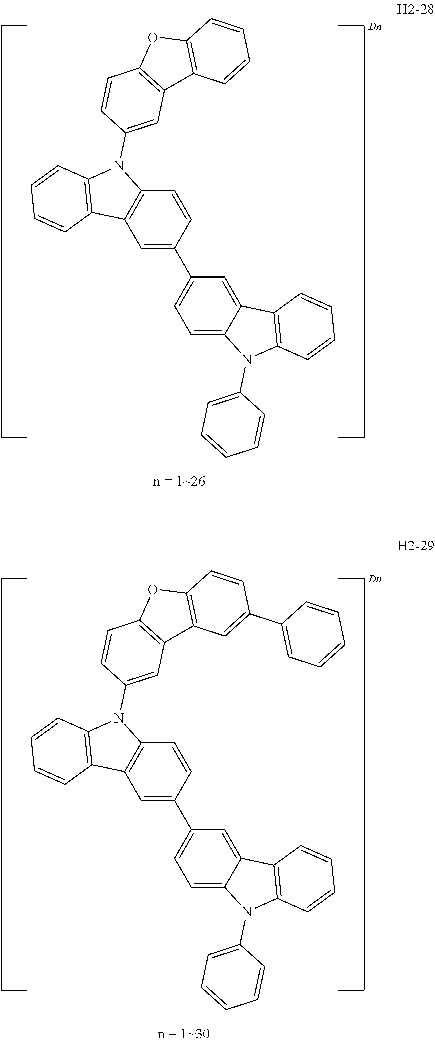 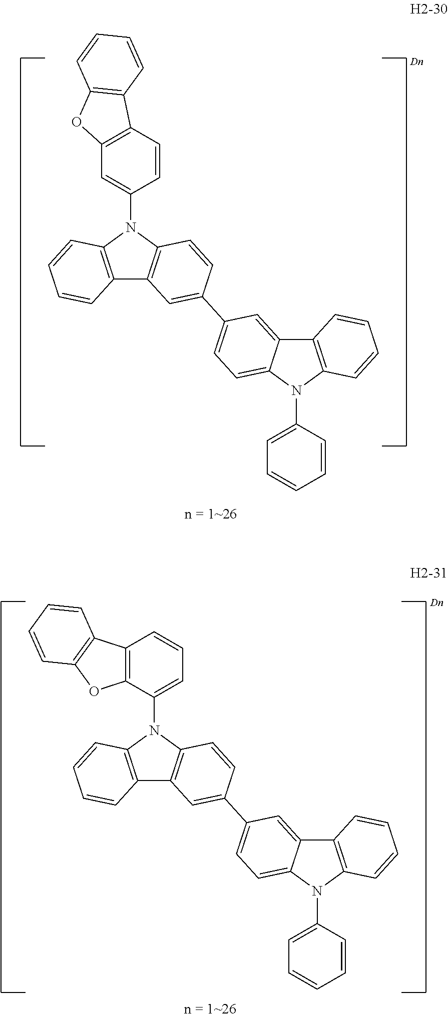 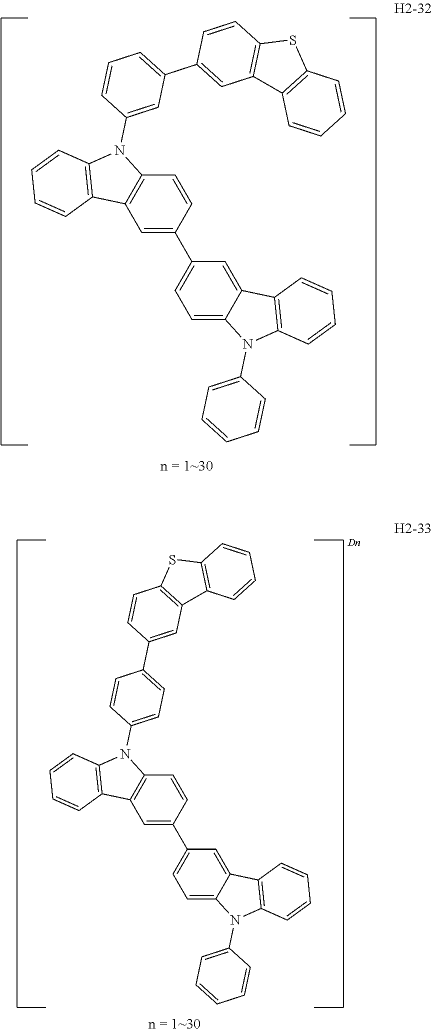 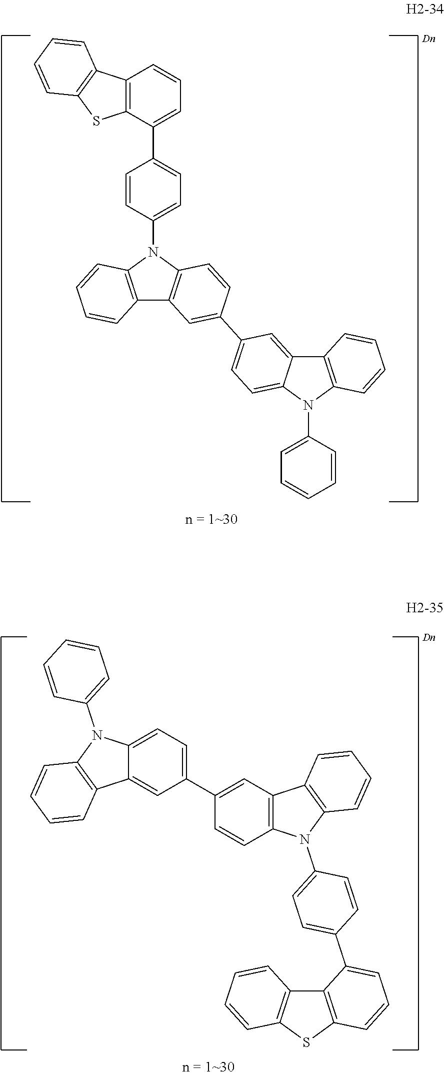 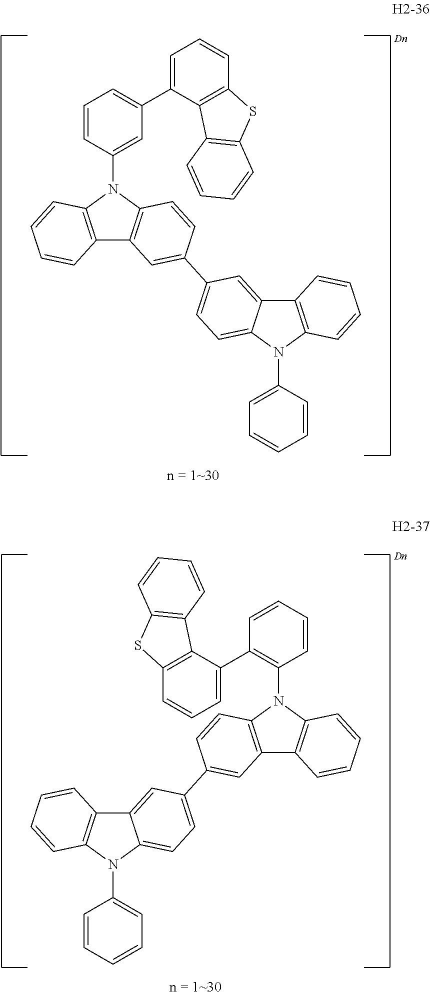 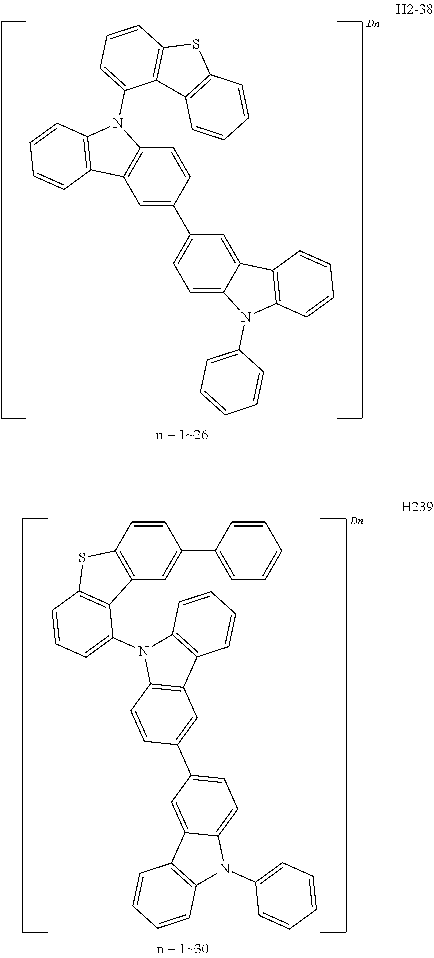 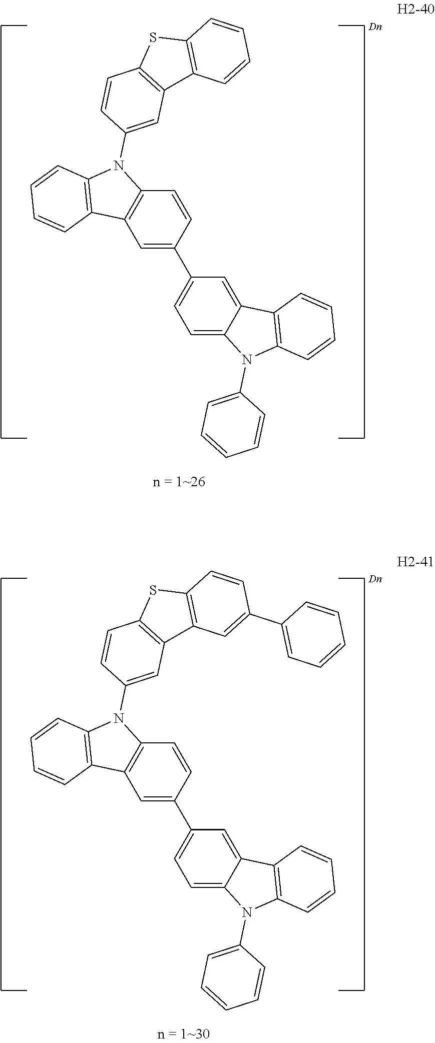 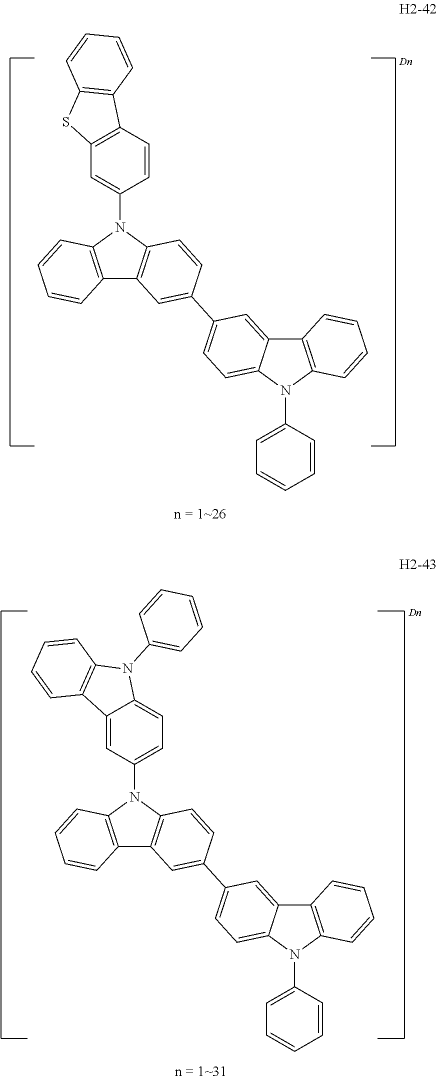 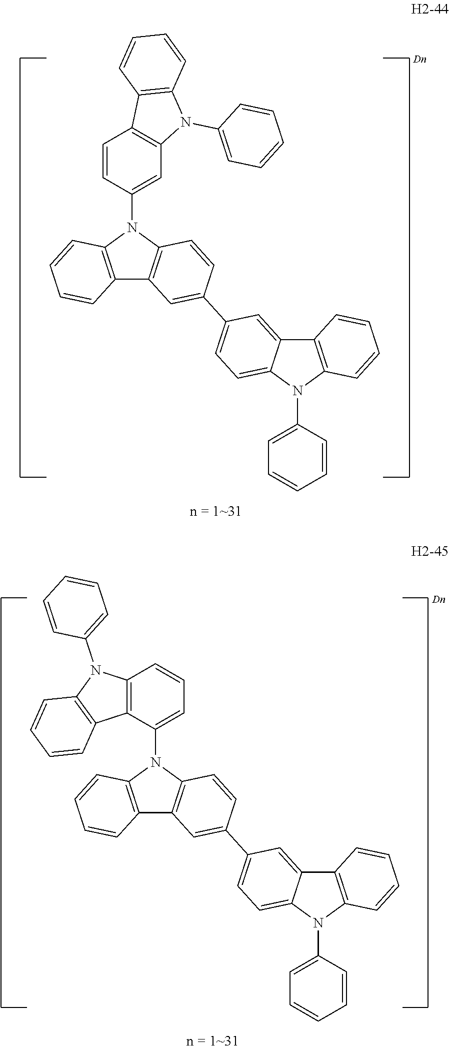 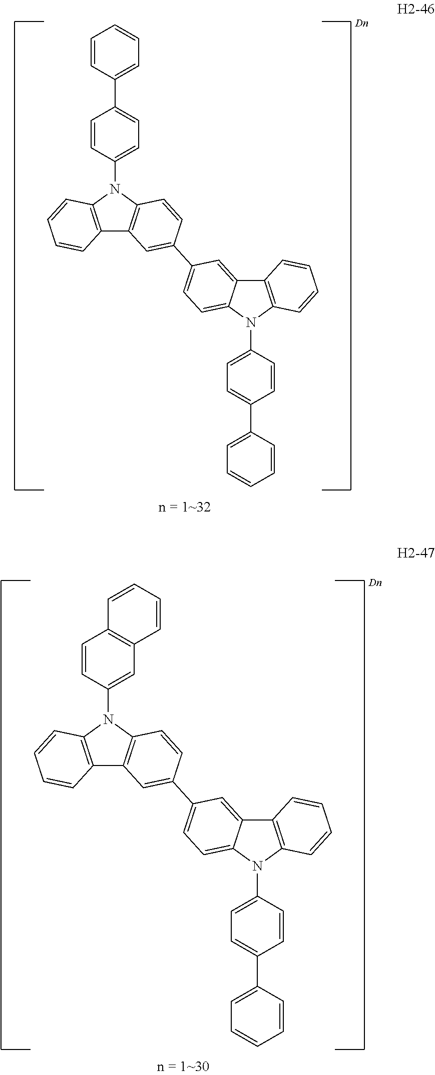 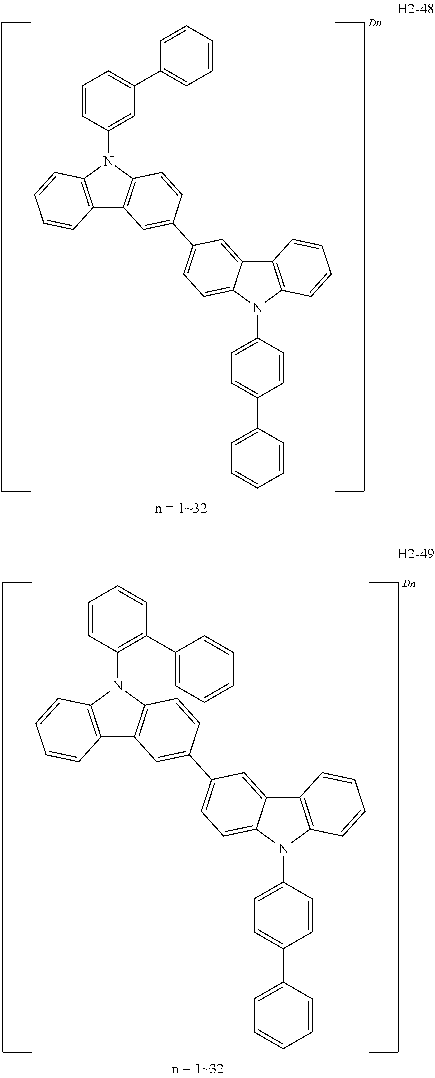 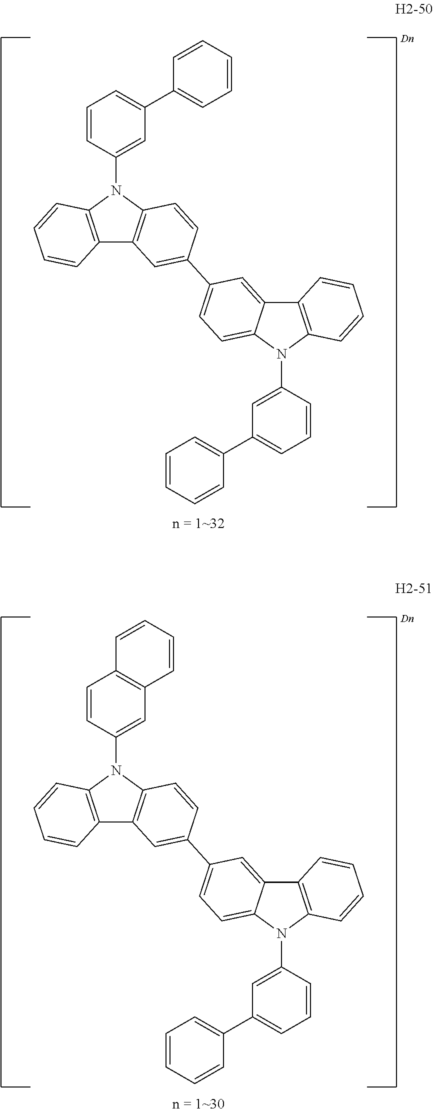 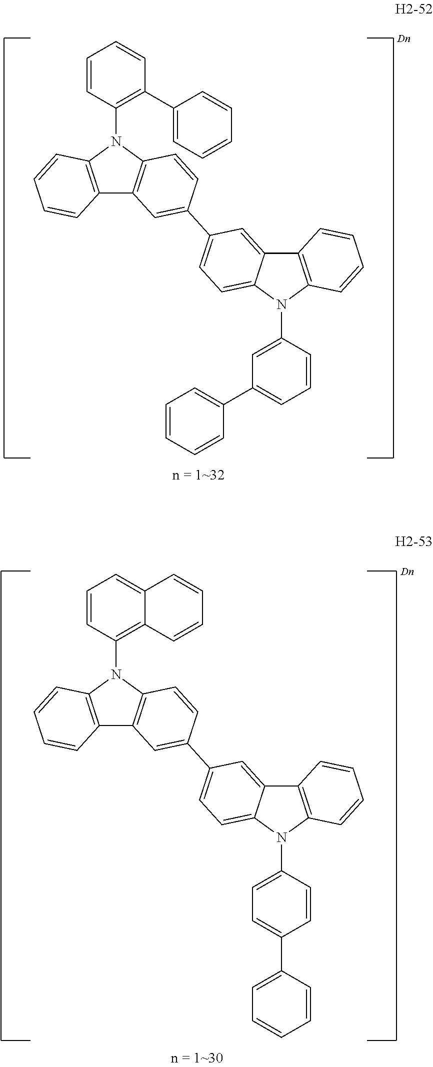 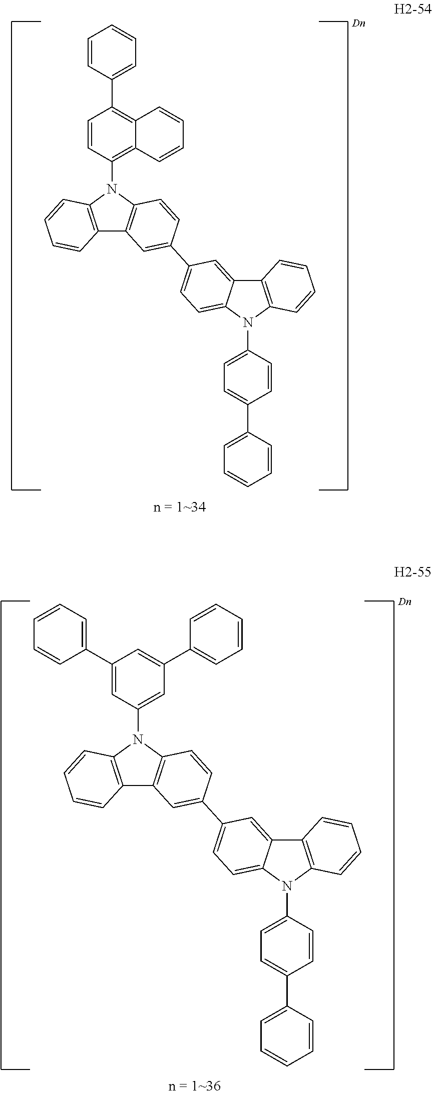 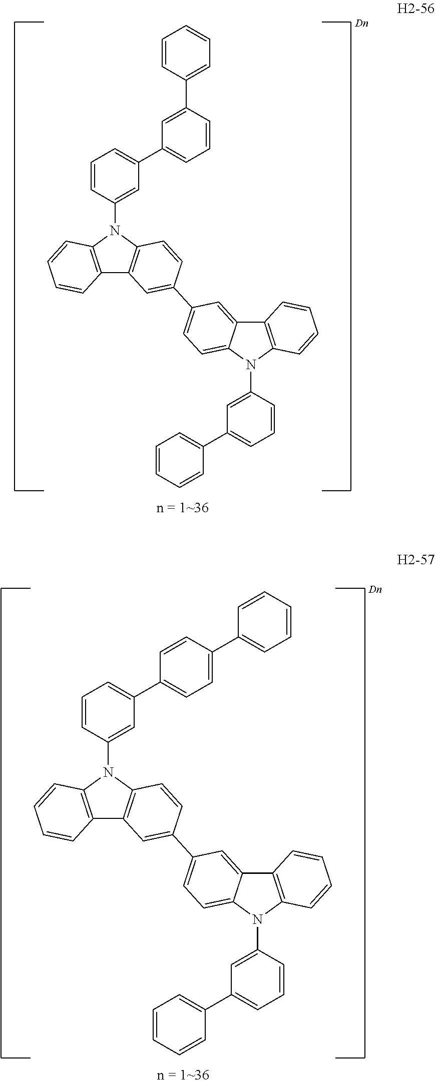 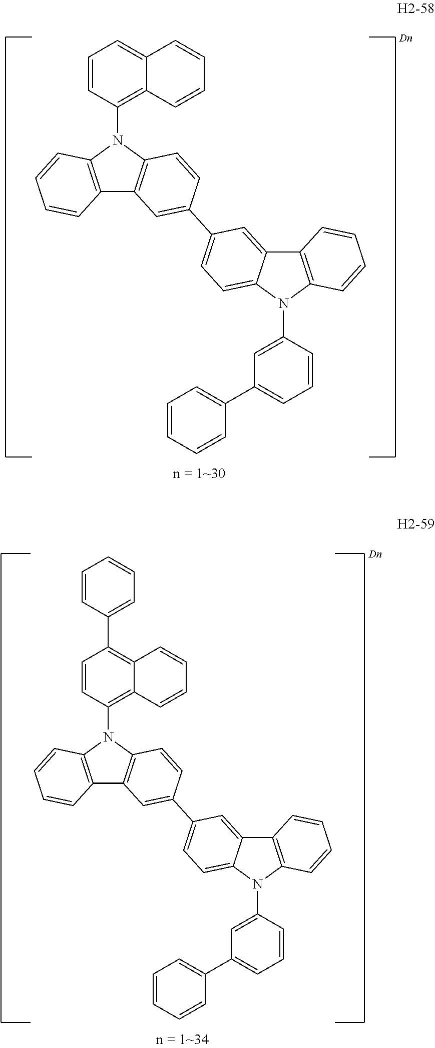 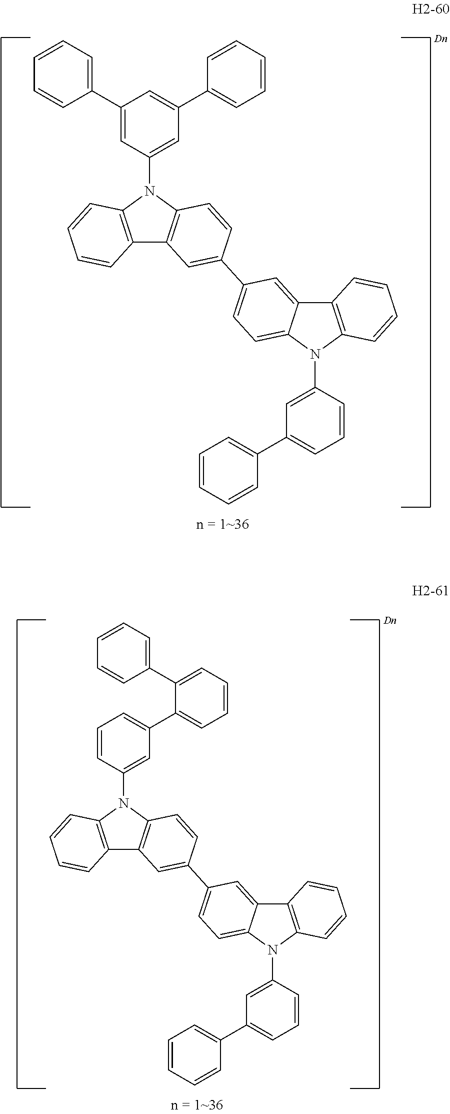 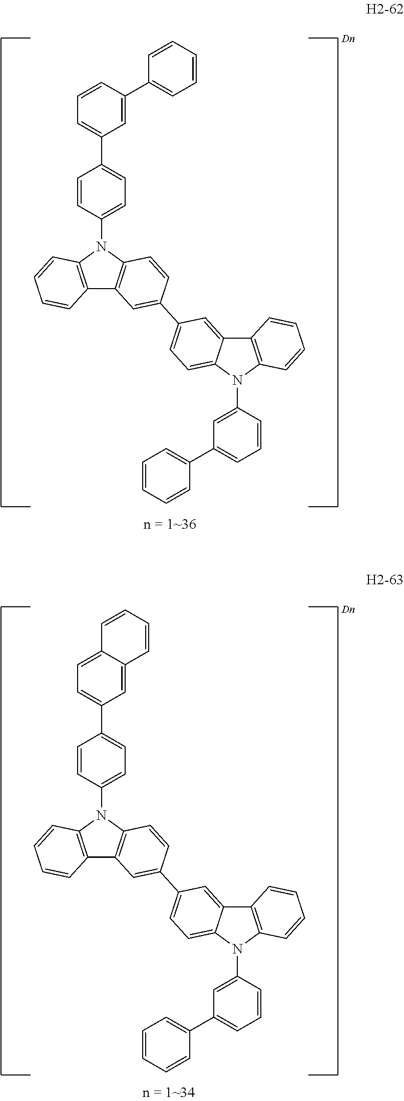 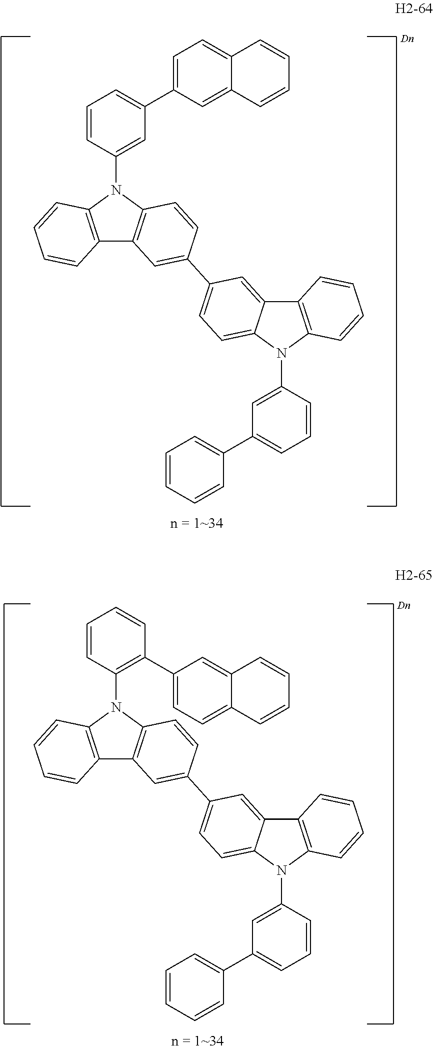 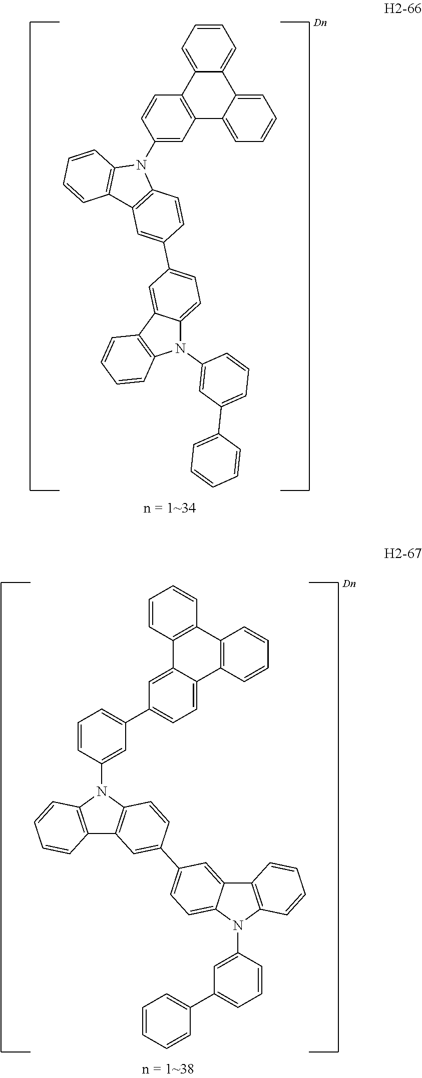 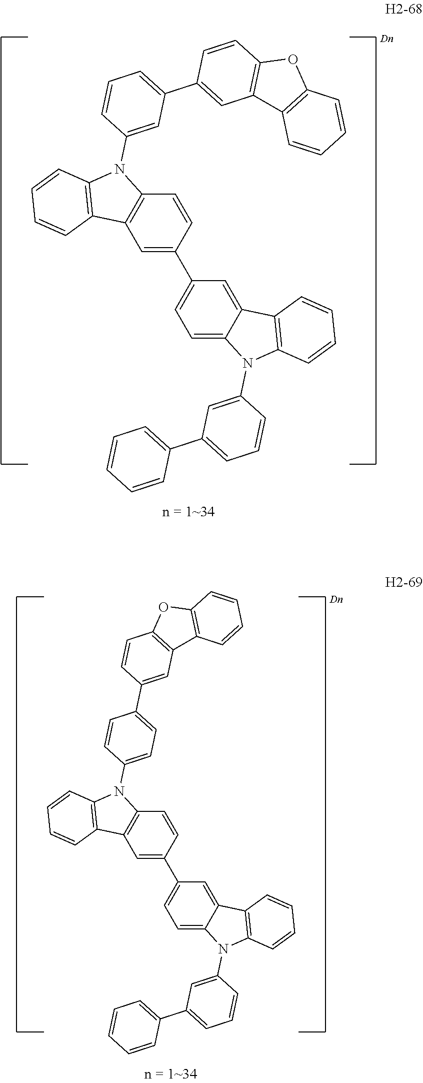 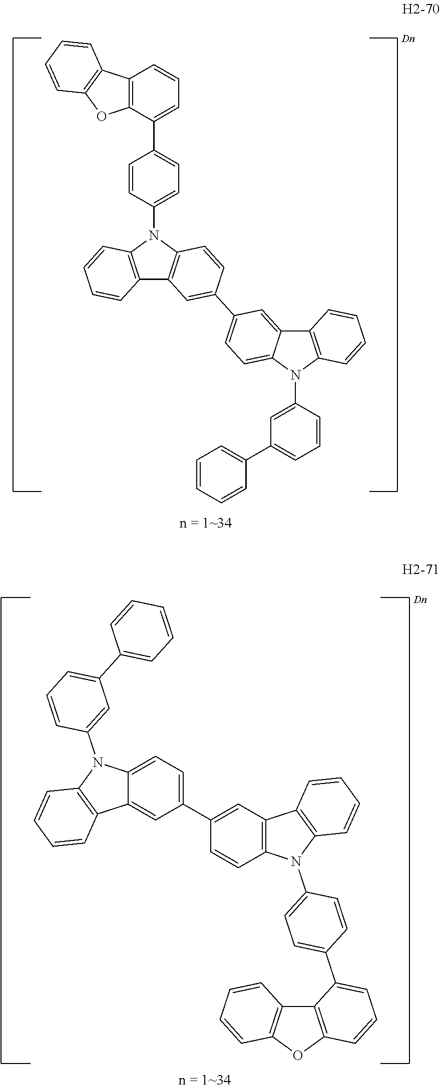 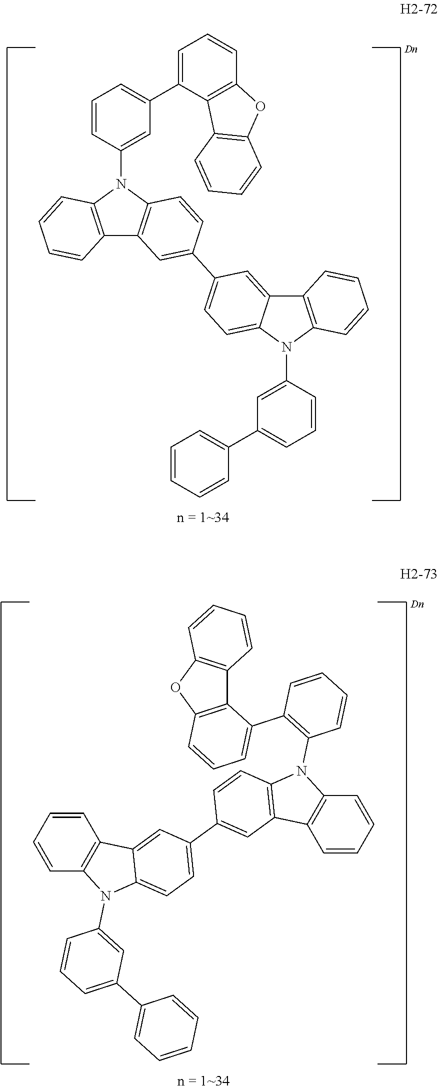 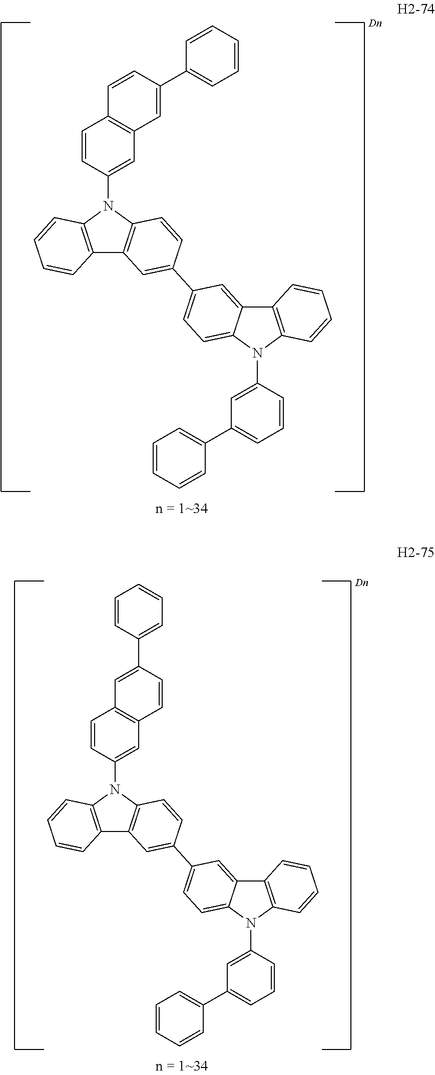 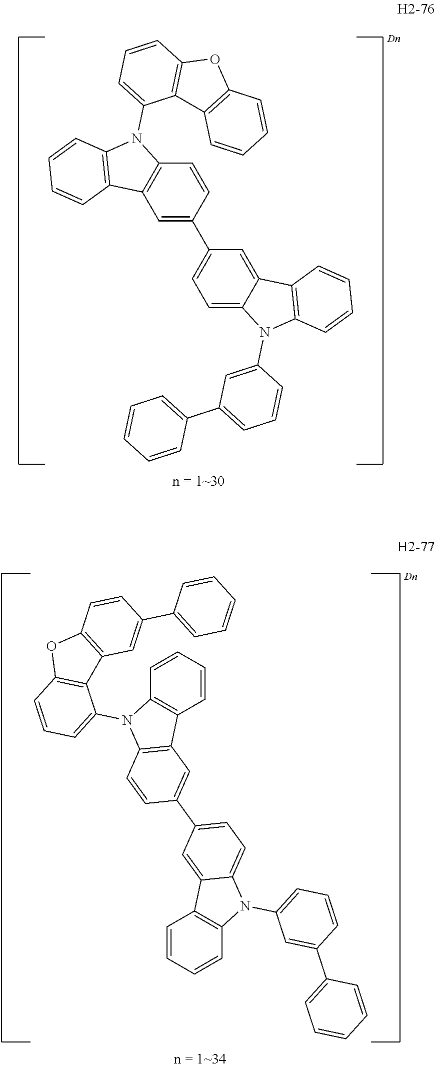 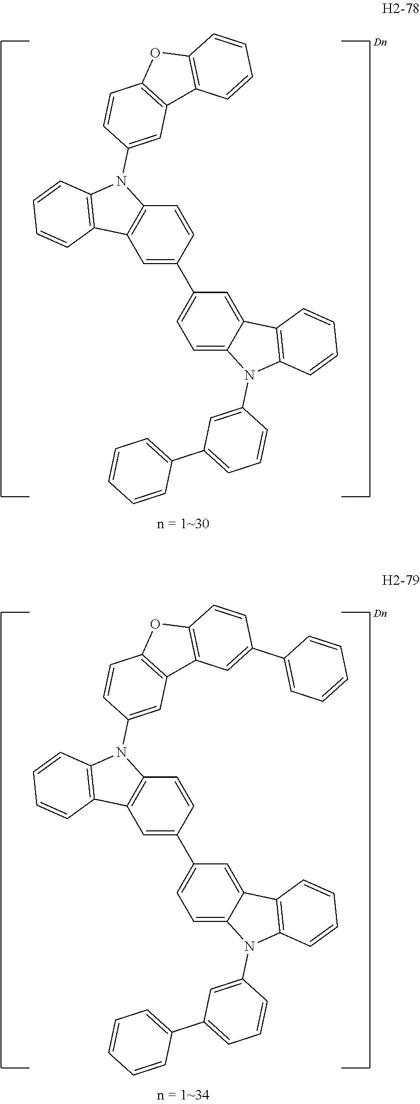 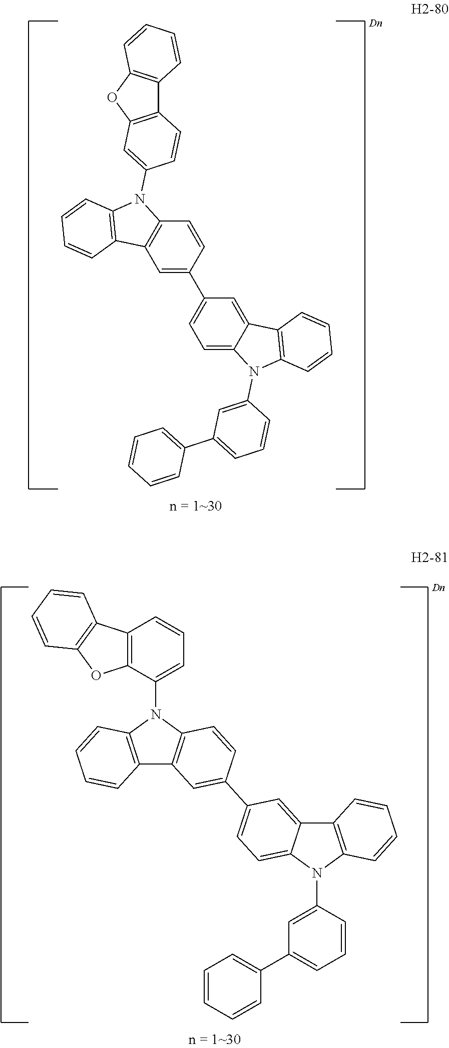 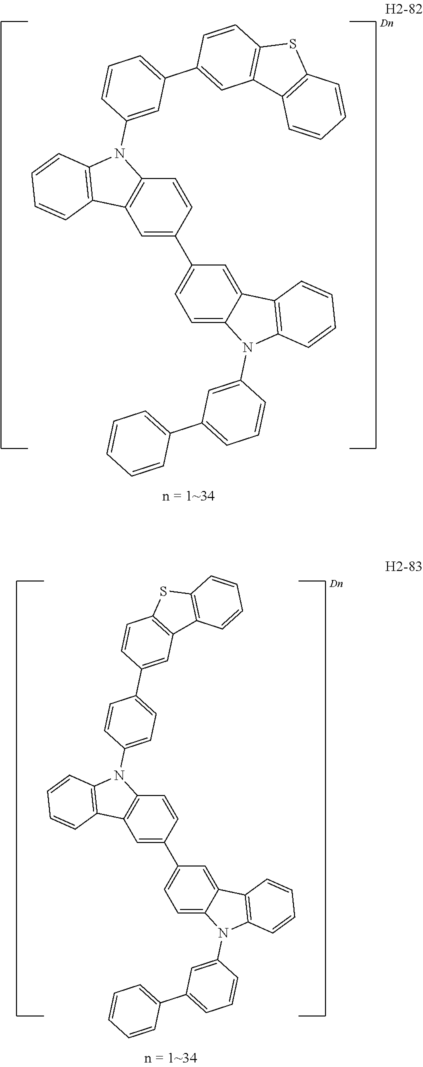 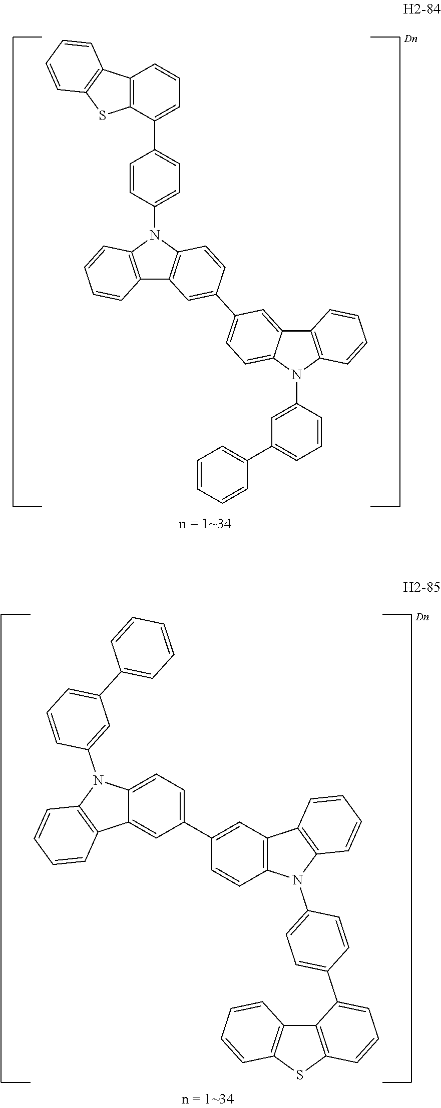 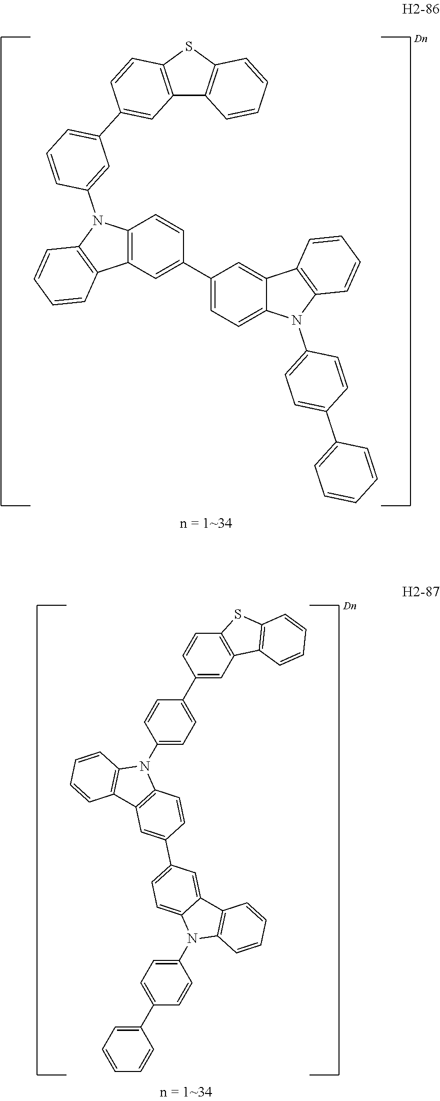 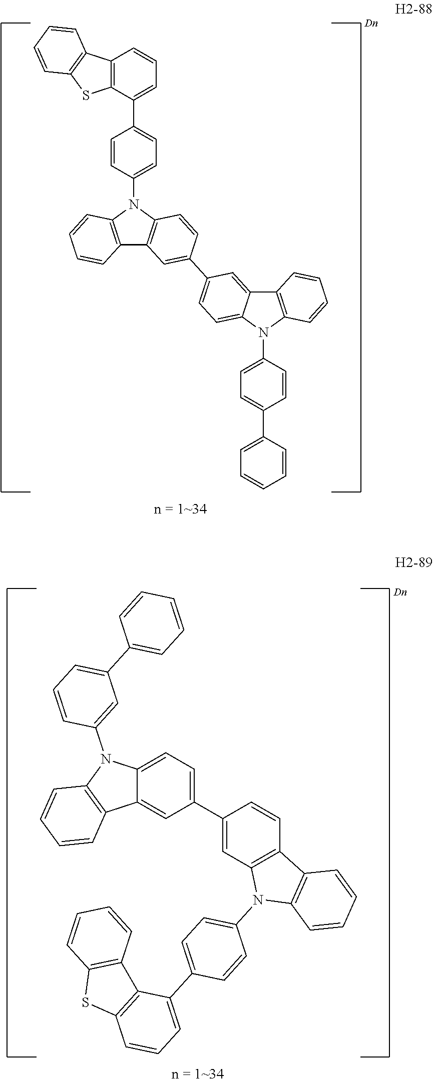 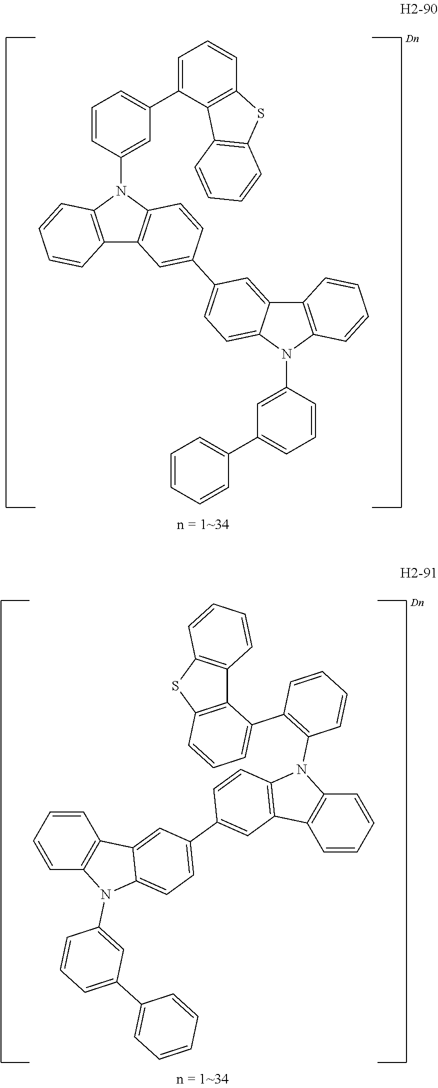 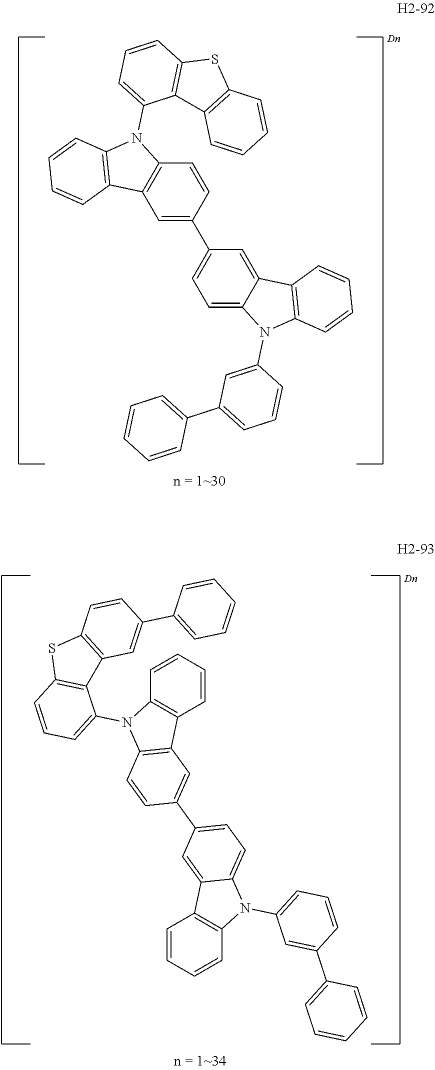 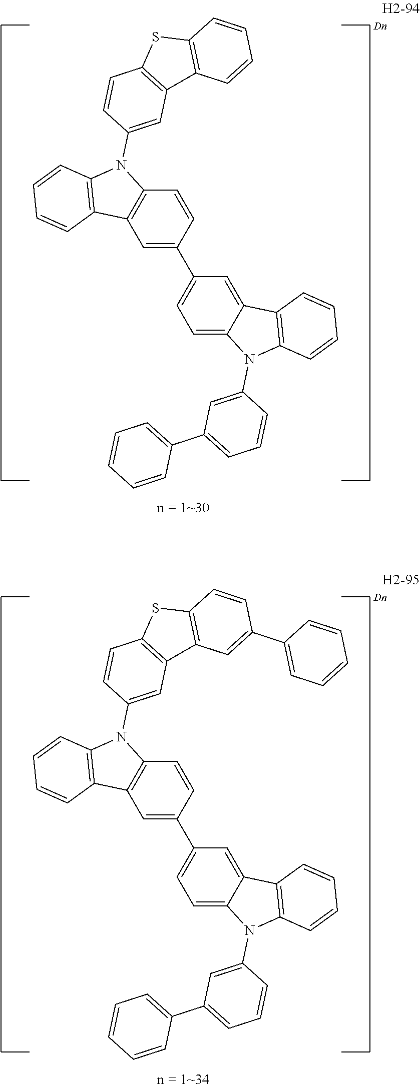 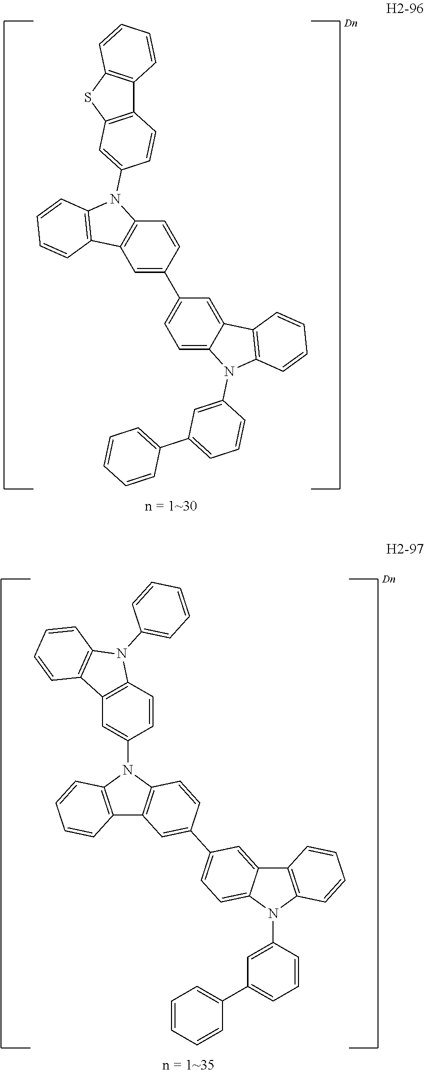 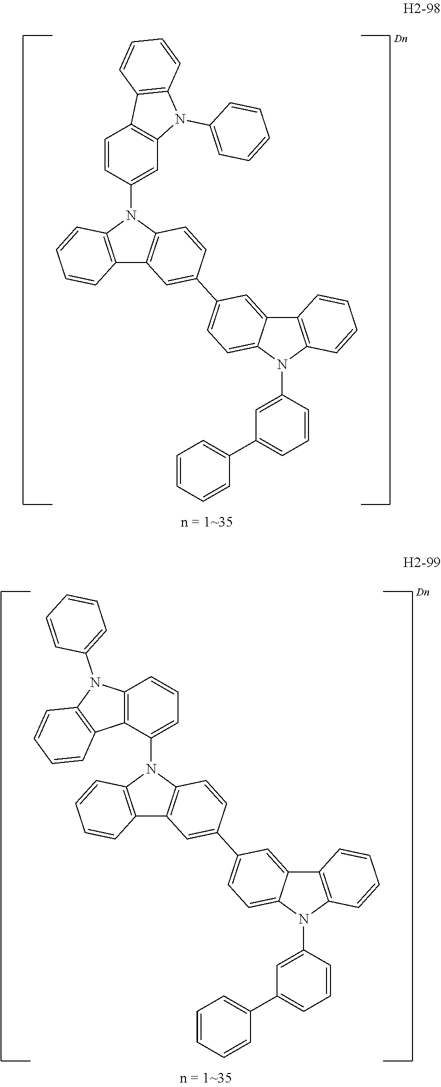 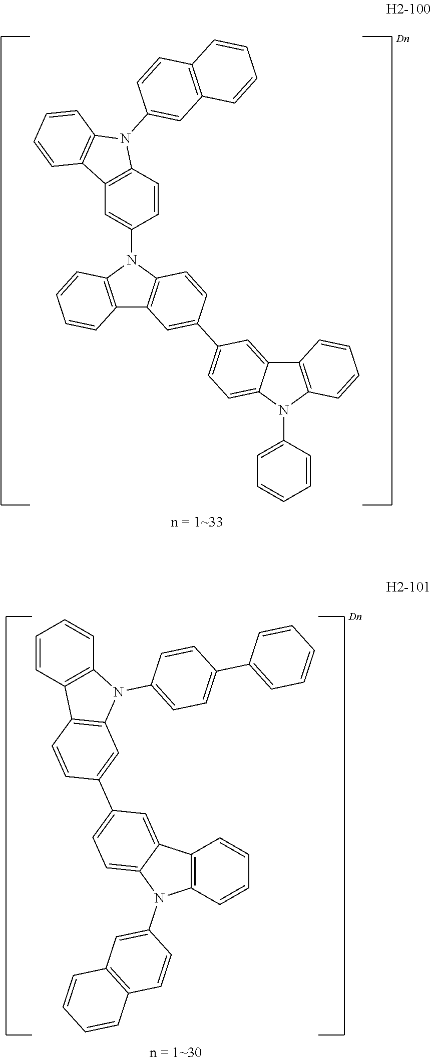 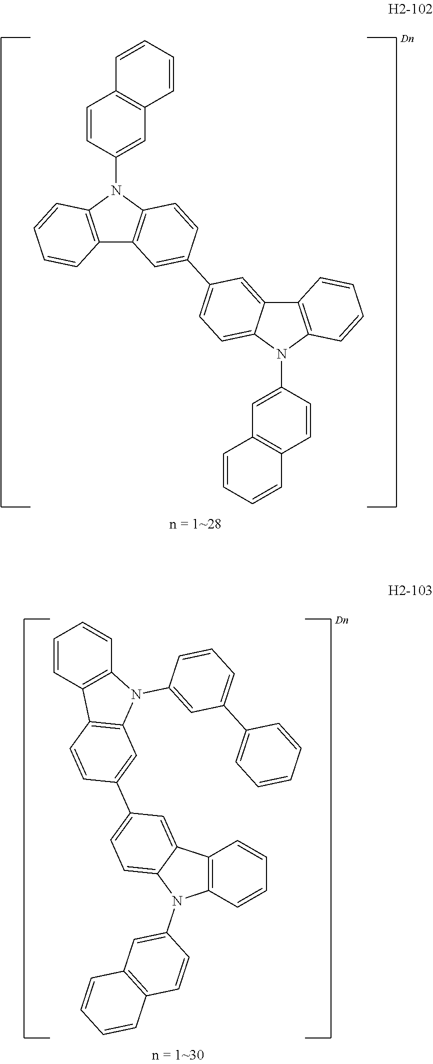 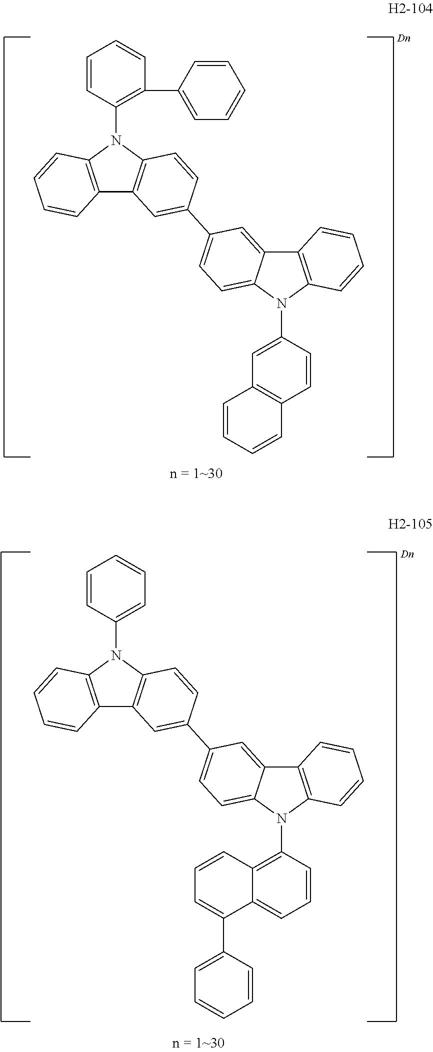 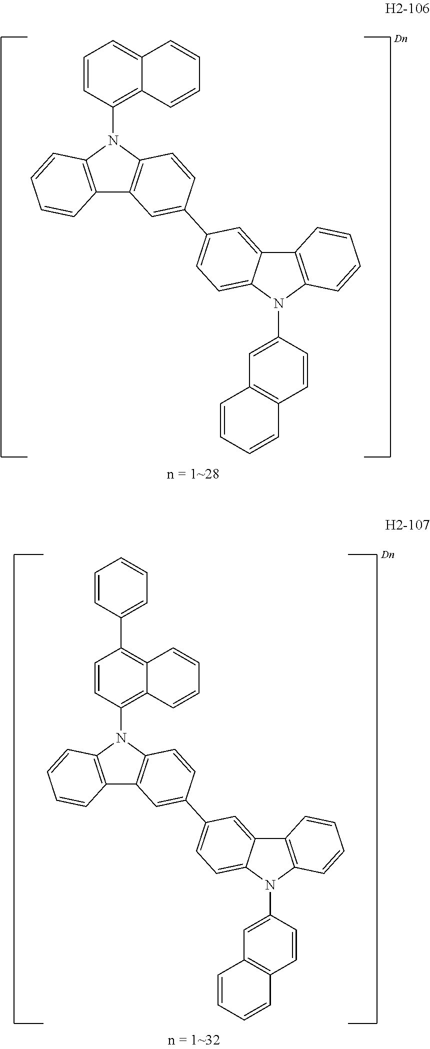 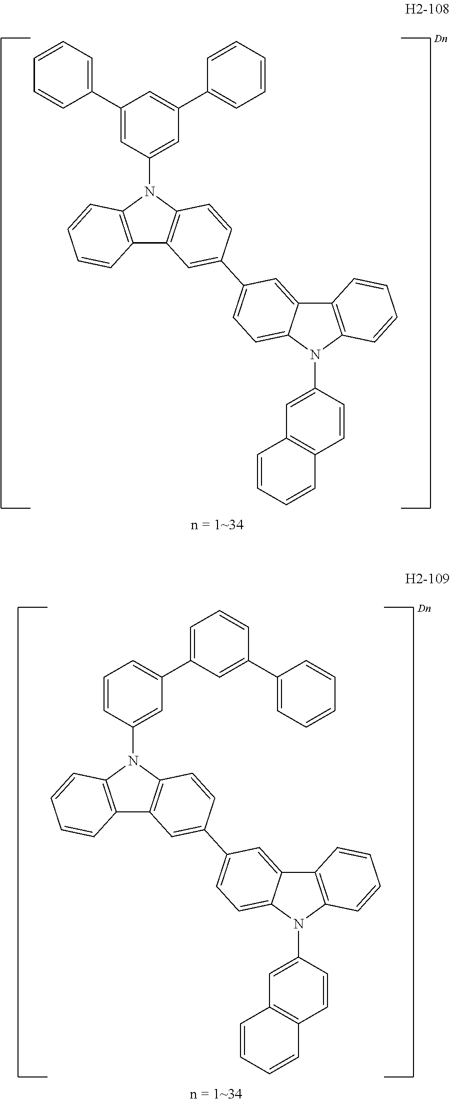 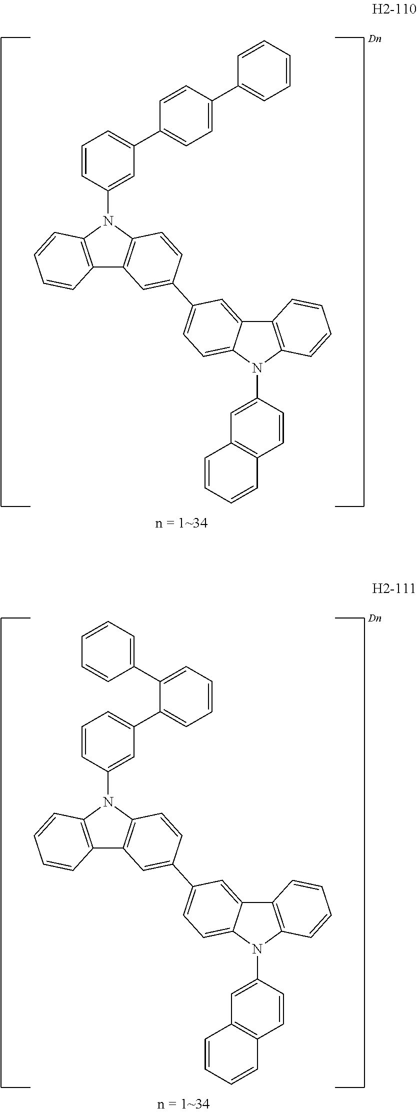 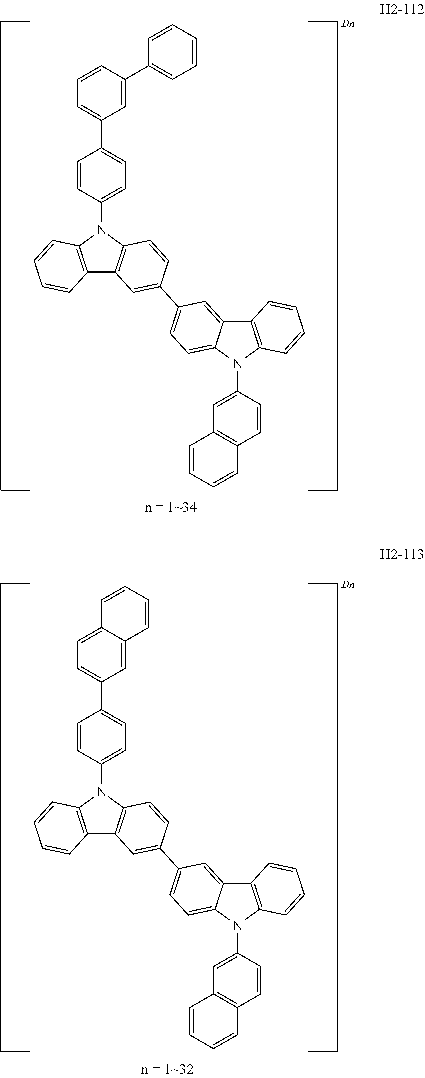 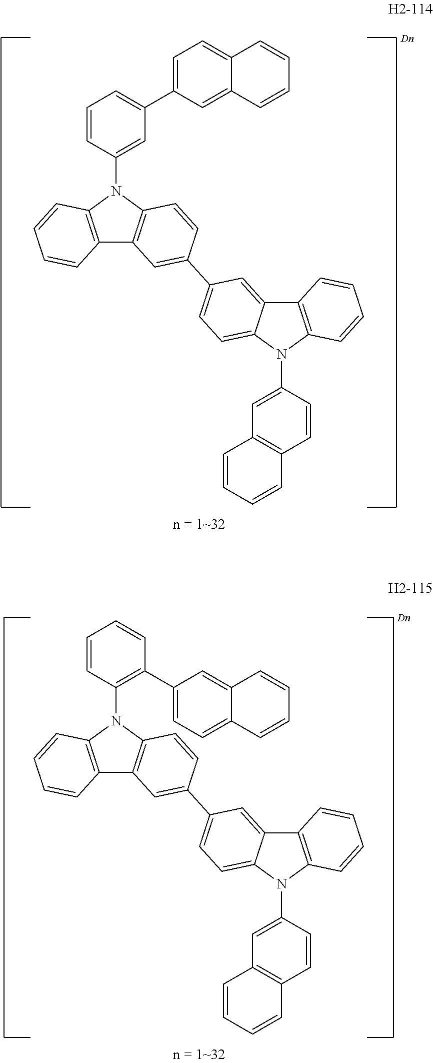 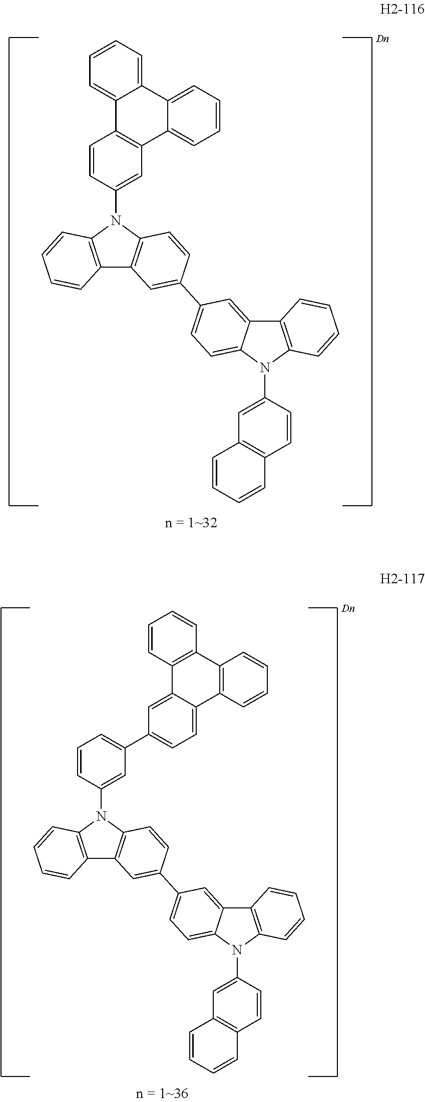 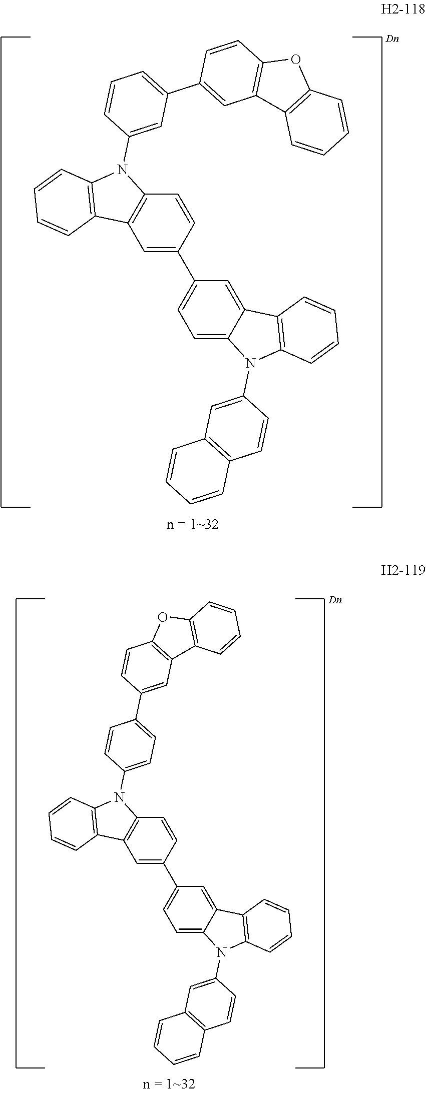 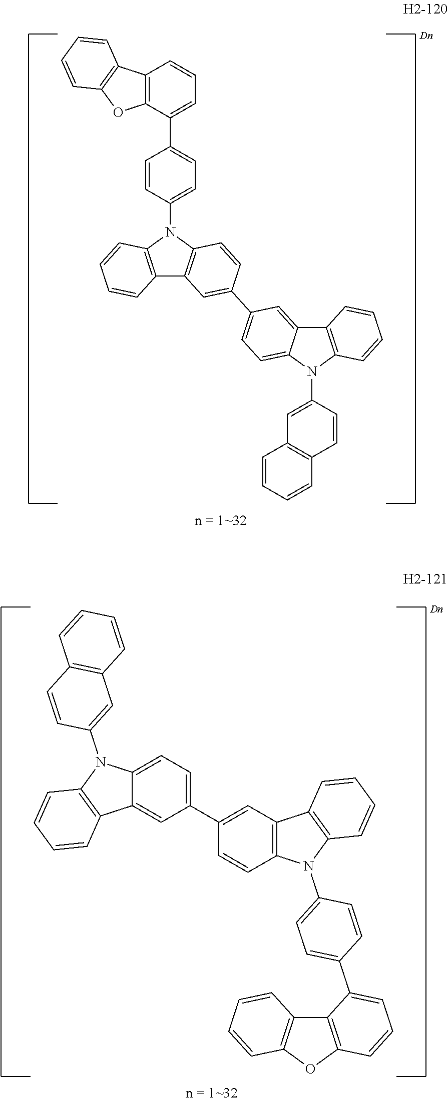 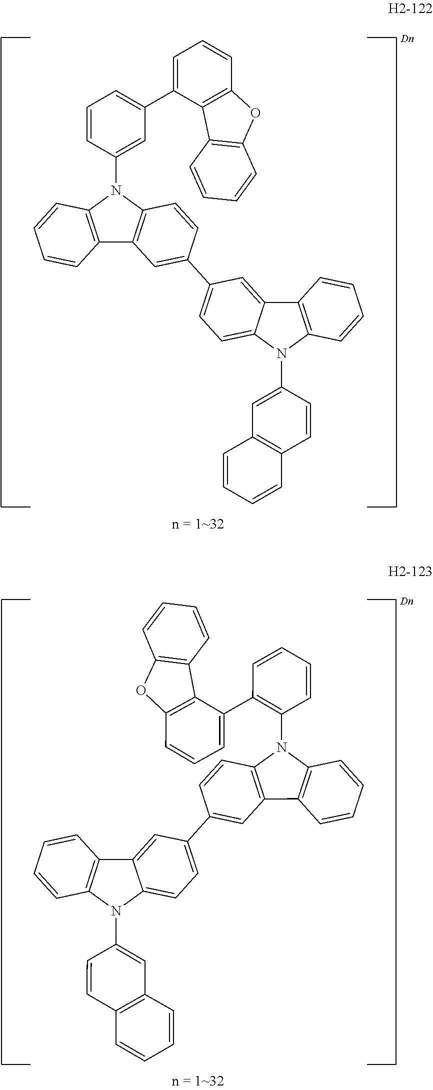 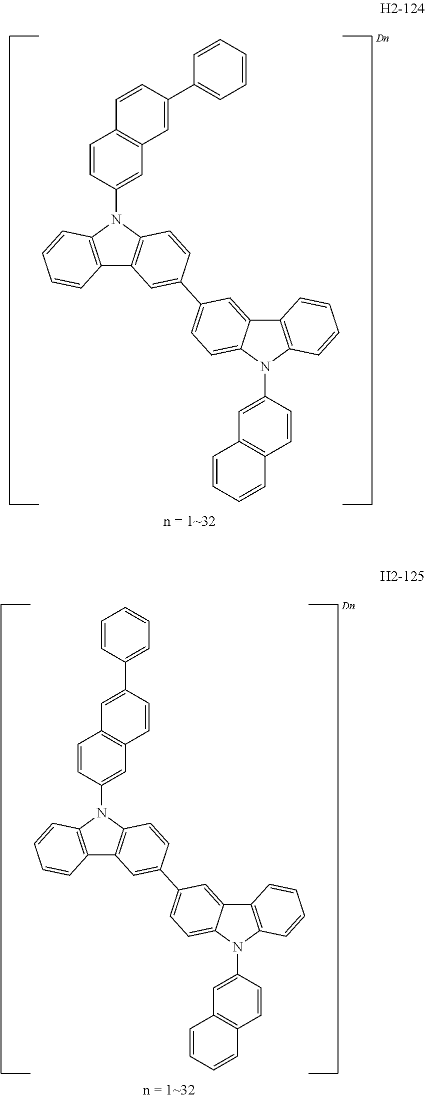 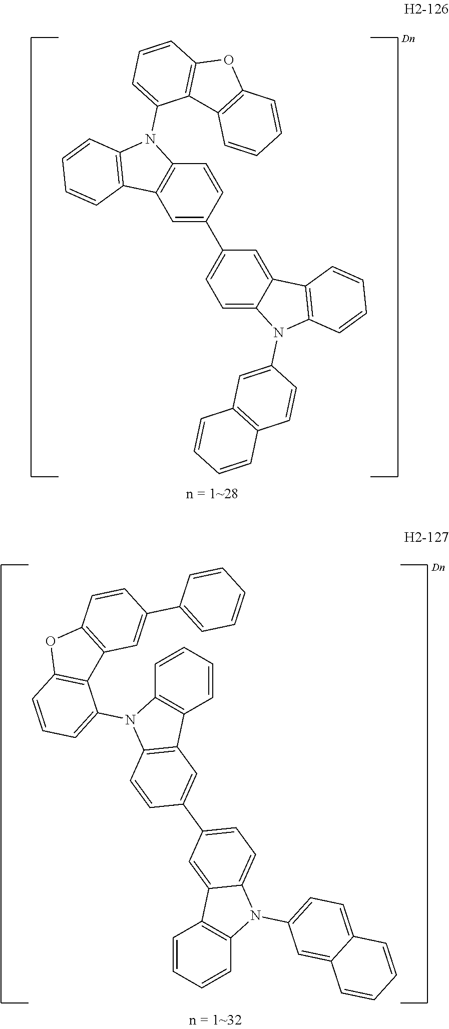 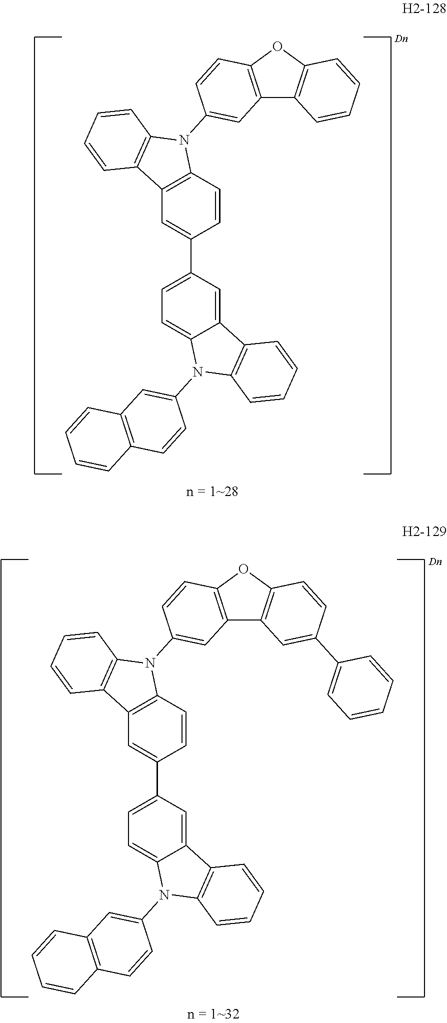 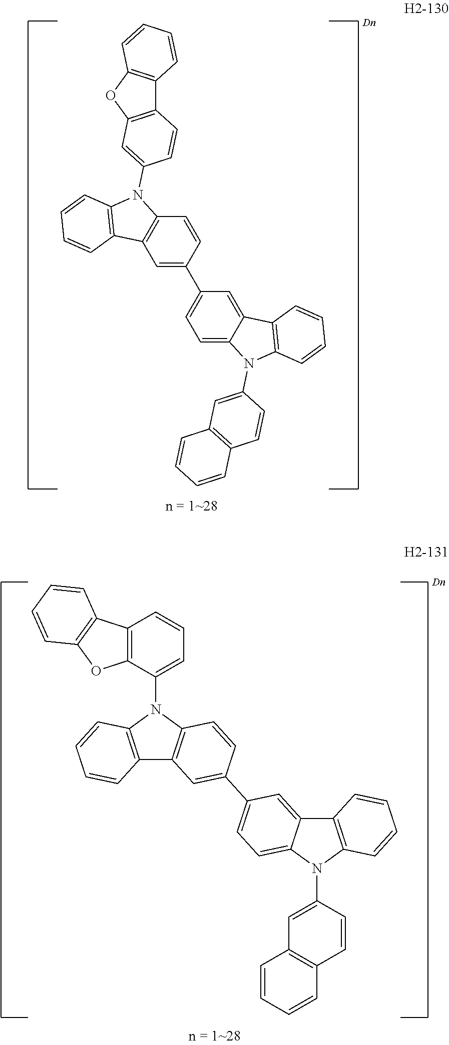 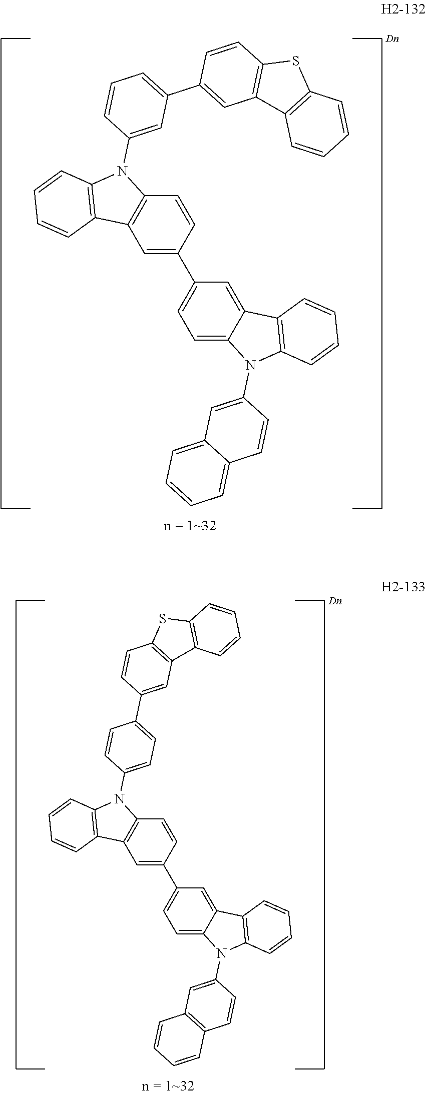 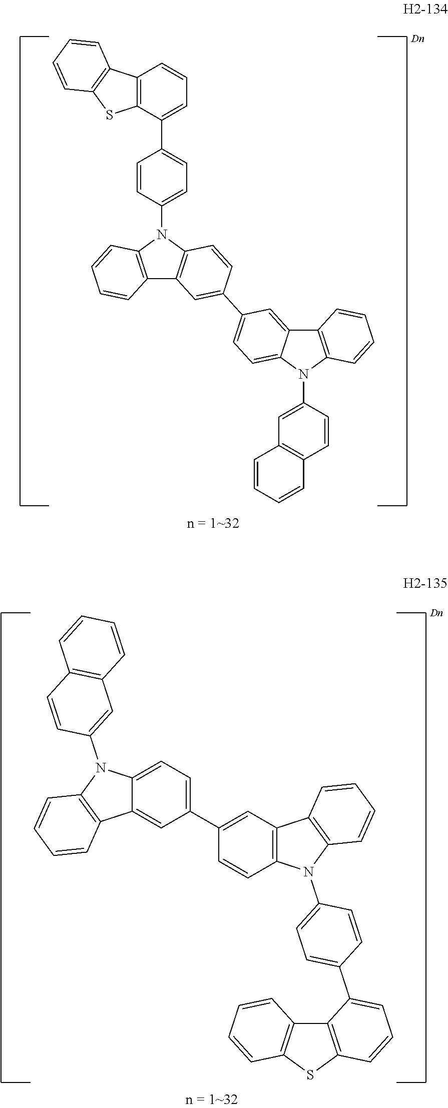 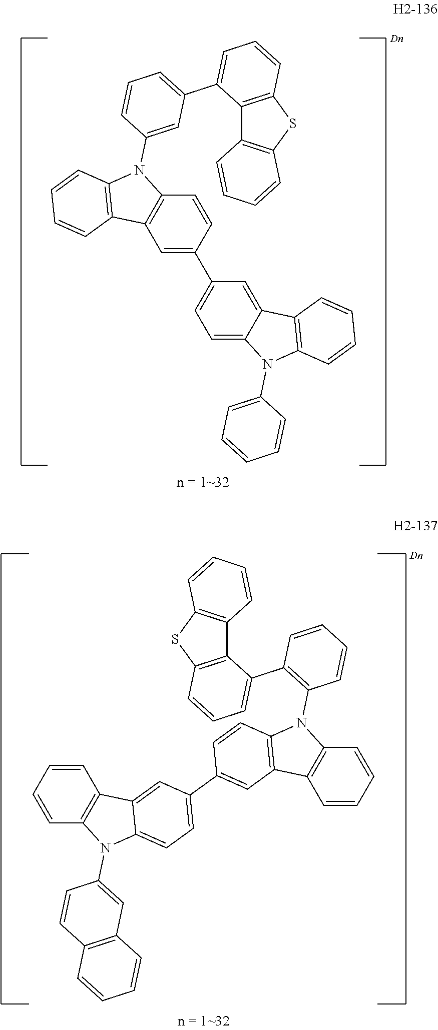 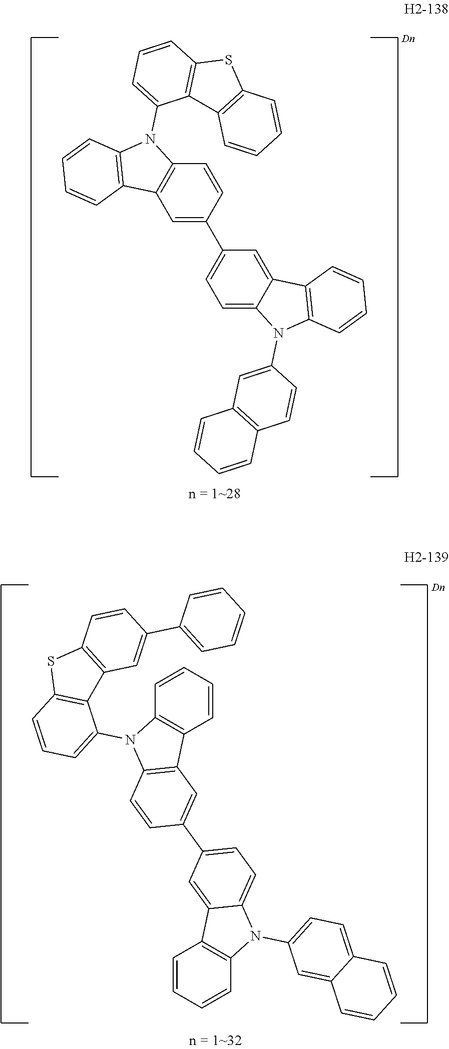 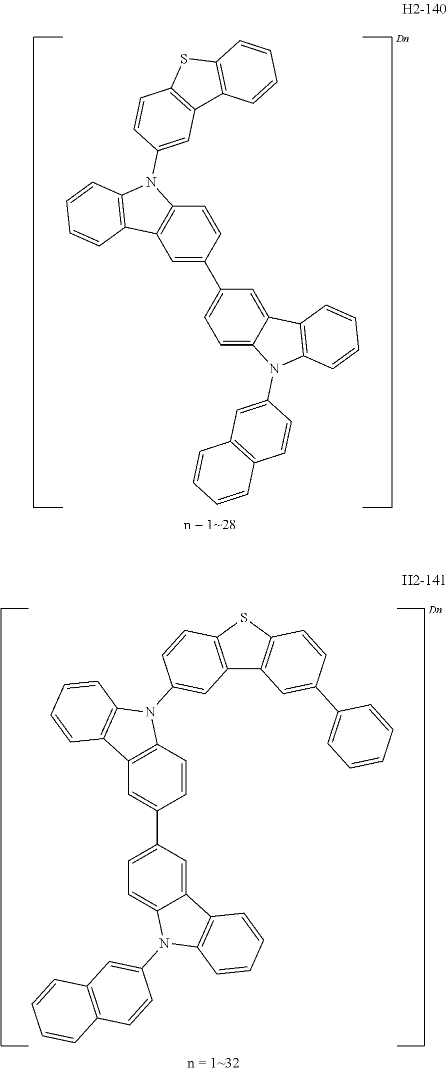 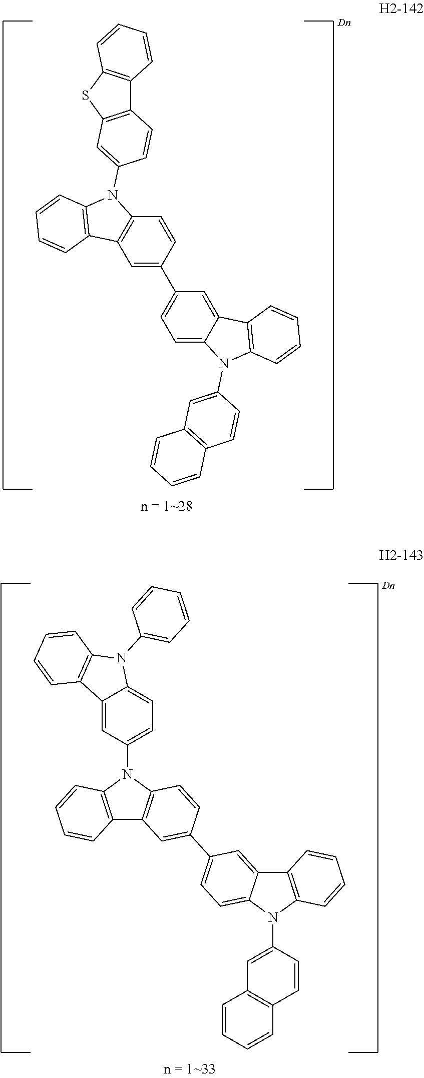 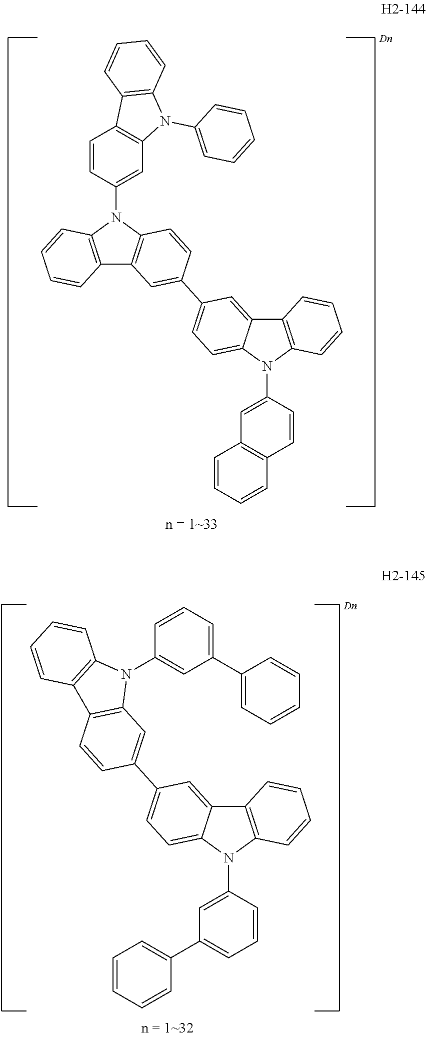 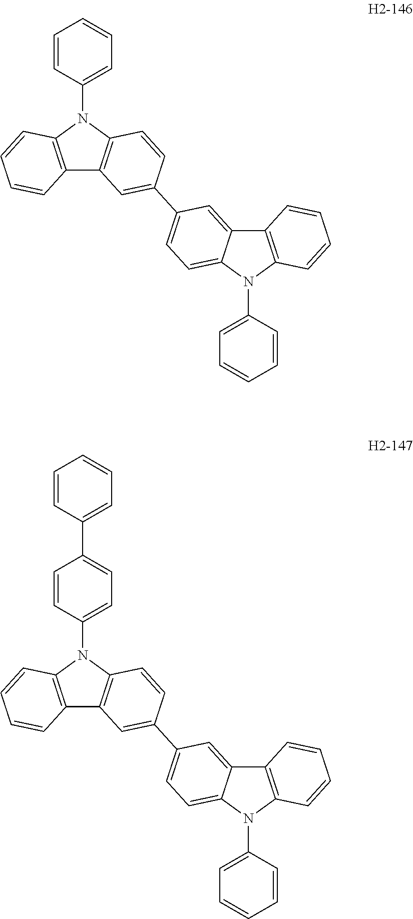 [SMILES: c1ccc(-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccccc5)ccc43)cc2)cc1] 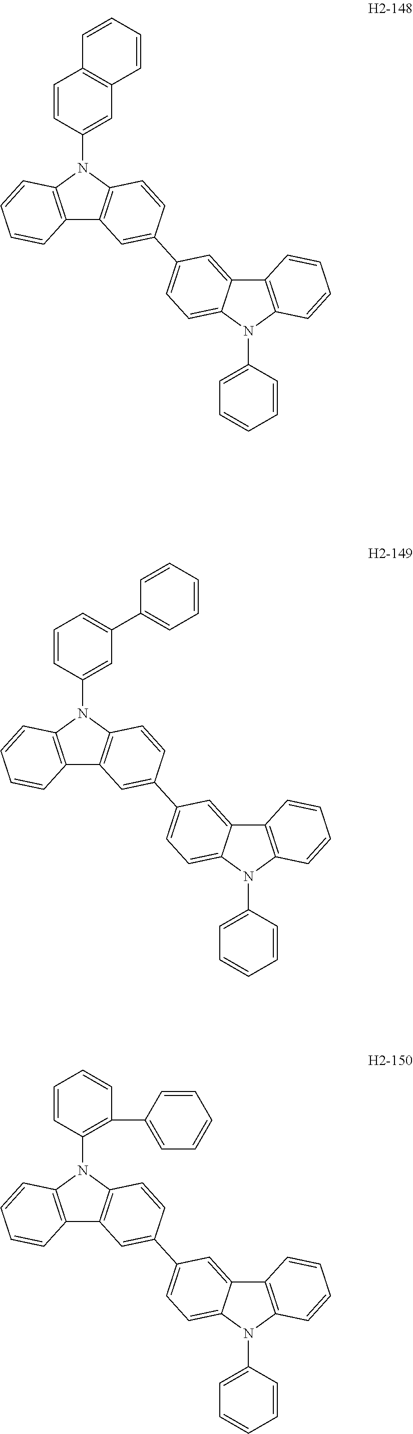 [SMILES: c1ccc(-n2c3ccccc3c3cc(-c4ccc5c(c4)c4ccccc4n5-c4ccc5ccccc5c4)ccc32)cc1 ; c1ccc(-c2cccc(-n3c4ccccc4c4cc(-c5ccc6c(c5)c5ccccc5n6-c5ccccc5)ccc43)c2)cc1 ; c1ccc(-c2ccccc2-n2c3ccccc3c3cc(-c4ccc5c(c4)c4ccccc4n5-c4ccccc4)ccc32)cc1] 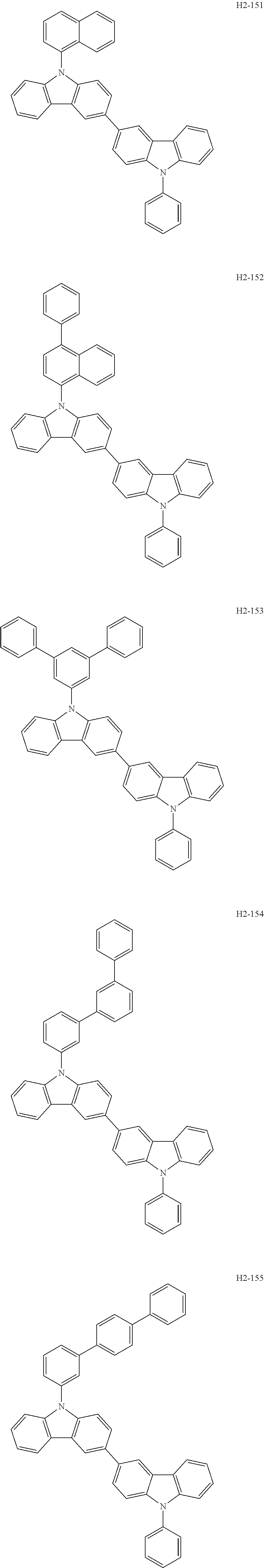 [SMILES: c1ccc(-n2c3ccccc3c3cc(-c4ccc5c(c4)c4ccccc4n5-c4cccc5ccccc45)ccc32)cc1 ; c1ccc(-c2ccc(-n3c4ccccc4c4cc(-c5ccc6c(c5)c5ccccc5n6-c5ccccc5)ccc43)c3ccccc23)cc1 ; c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-c2cccc(-c3cccc(-n4c5ccccc5c5cc(-c6ccc7c(c6)c6ccccc6n7-c6ccccc6)ccc54)c3)c2)cc1 ; c1ccc(-c2ccc(-c3cccc(-n4c5ccccc5c5cc(-c6ccc7c(c6)c6ccccc6n7-c6ccccc6)ccc54)c3)cc2)cc1] 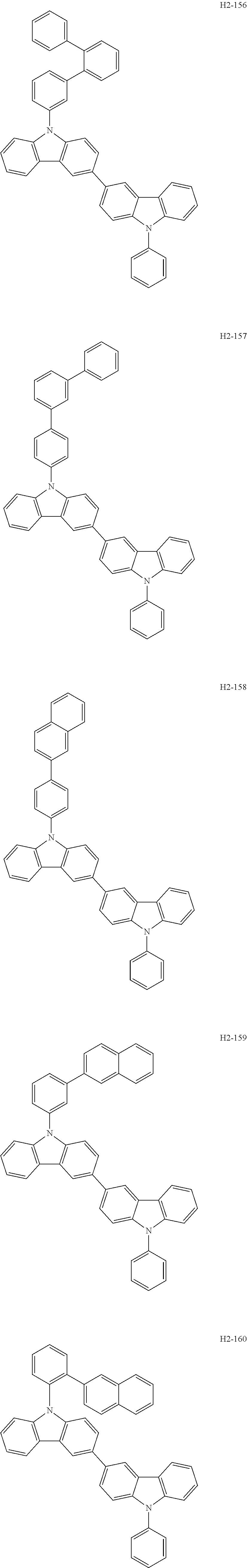 [SMILES: c1ccc(-c2ccccc2-c2cccc(-n3c4ccccc4c4cc(-c5ccc6c(c5)c5ccccc5n6-c5ccccc5)ccc43)c2)cc1 ; c1ccc(-c2cccc(-c3ccc(-n4c5ccccc5c5cc(-c6ccc7c(c6)c6ccccc6n7-c6ccccc6)ccc54)cc3)c2)cc1 ; c1ccc(-n2c3ccccc3c3cc(-c4ccc5c(c4)c4ccccc4n5-c4ccc(-c5ccc6ccccc6c5)cc4)ccc32)cc1 ; c1ccc(-n2c3ccccc3c3cc(-c4ccc5c(c4)c4ccccc4n5-c4cccc(-c5ccc6ccccc6c5)c4)ccc32)cc1 ; c1ccc(-n2c3ccccc3c3cc(-c4ccc5c(c4)c4ccccc4n5-c4ccccc4-c4ccc5ccccc5c4)ccc32)cc1] 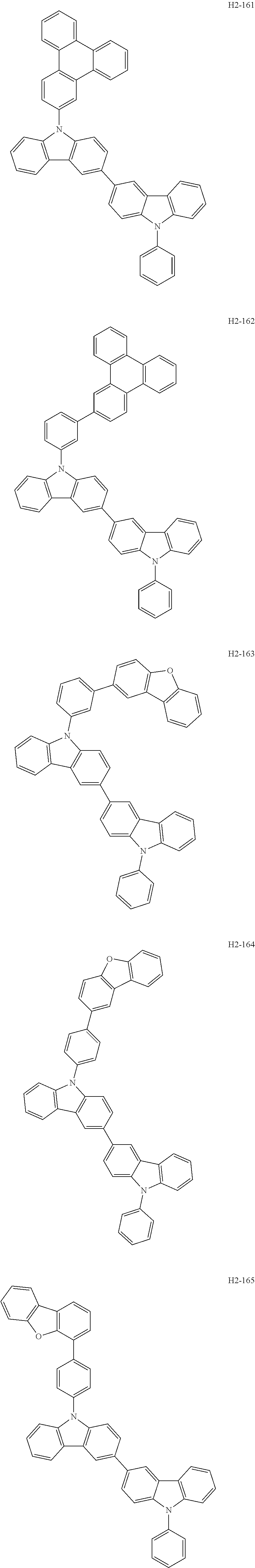 [SMILES: c1ccc(-n2c3ccccc3c3cc(-c4ccc5c(c4)c4ccccc4n5-c4ccc5c6ccccc6c6ccccc6c5c4)ccc32)cc1 ; c1ccc(-n2c3ccccc3c3cc(-c4ccc5c(c4)c4ccccc4n5-c4cccc(-c5ccc6c7ccccc7c7ccccc7c6c5)c4)ccc32)cc1 ; c1ccc(-n2c3ccccc3c3cc(-c4ccc5c(c4)c4ccccc4n5-c4cccc(-c5ccc6oc7ccccc7c6c5)c4)ccc32)cc1 ; c1ccc(-n2c3ccccc3c3cc(-c4ccc5c(c4)c4ccccc4n5-c4ccc(-c5ccc6oc7ccccc7c6c5)cc4)ccc32)cc1 ; c1ccc(-n2c3ccccc3c3cc(-c4ccc5c(c4)c4ccccc4n5-c4ccc(-c5cccc6c5oc5ccccc56)cc4)ccc32)cc1] 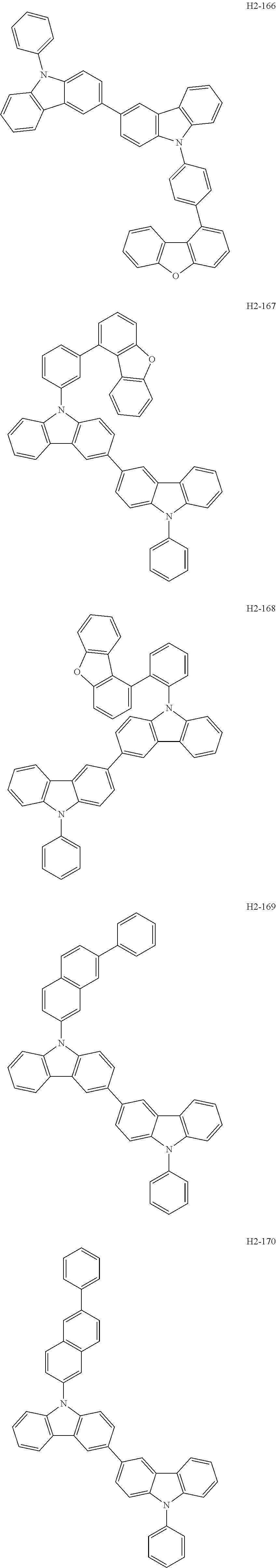 [SMILES: c1ccc(-n2c3ccccc3c3cc(-c4ccc5c(c4)c4ccccc4n5-c4ccc(-c5cccc6oc7ccccc7c56)cc4)ccc32)cc1 ; c1ccc(-n2c3ccccc3c3cc(-c4ccc5c(c4)c4ccccc4n5-c4cccc(-c5cccc6oc7ccccc7c56)c4)ccc32)cc1 ; c1ccc(-n2c3ccccc3c3cc(-c4ccc5c(c4)c4ccccc4n5-c4ccccc4-c4cccc5oc6ccccc6c45)ccc32)cc1 ; c1ccc(-c2ccc3ccc(-n4c5ccccc5c5cc(-c6ccc7c(c6)c6ccccc6n7-c6ccccc6)ccc54)cc3c2)cc1 ; c1ccc(-c2ccc3cc(-n4c5ccccc5c5cc(-c6ccc7c(c6)c6ccccc6n7-c6ccccc6)ccc54)ccc3c2)cc1] 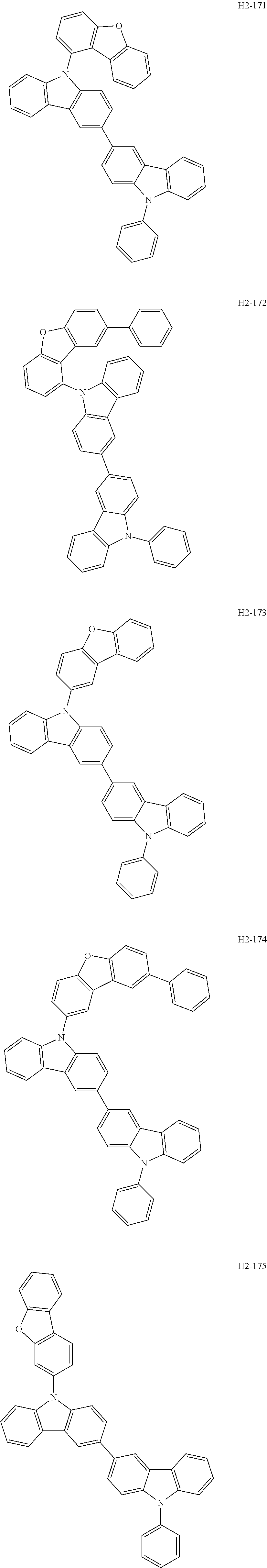 [SMILES: c1ccc(-n2c3ccccc3c3cc(-c4ccc5c(c4)c4ccccc4n5-c4cccc5oc6ccccc6c45)ccc32)cc1 ; c1ccc(-c2ccc3oc4cccc(-n5c6ccccc6c6cc(-c7ccc8c(c7)c7ccccc7n8-c7ccccc7)ccc65)c4c3c2)cc1 ; c1ccc(-n2c3ccccc3c3cc(-c4ccc5c(c4)c4ccccc4n5-c4ccc5oc6ccccc6c5c4)ccc32)cc1 ; c1ccc(-c2ccc3oc4ccc(-n5c6ccccc6c6cc(-c7ccc8c(c7)c7ccccc7n8-c7ccccc7)ccc65)cc4c3c2)cc1 ; c1ccc(-n2c3ccccc3c3cc(-c4ccc5c(c4)c4ccccc4n5-c4ccc5c(c4)oc4ccccc45)ccc32)cc1] 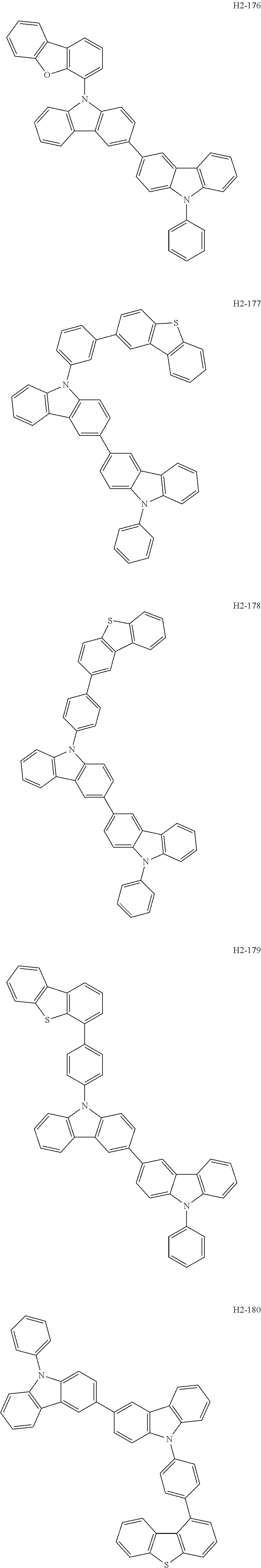 [SMILES: c1ccc(-n2c3ccccc3c3cc(-c4ccc5c(c4)c4ccccc4n5-c4cccc5c4oc4ccccc45)ccc32)cc1 ; c1ccc(-n2c3ccccc3c3cc(-c4ccc5c(c4)c4ccccc4n5-c4cccc(-c5ccc6sc7ccccc7c6c5)c4)ccc32)cc1 ; c1ccc(-n2c3ccccc3c3cc(-c4ccc5c(c4)c4ccccc4n5-c4ccc(-c5ccc6sc7ccccc7c6c5)cc4)ccc32)cc1 ; c1ccc(-n2c3ccccc3c3cc(-c4ccc5c(c4)c4ccccc4n5-c4ccc(-c5cccc6c5sc5ccccc56)cc4)ccc32)cc1 ; c1ccc(-n2c3ccccc3c3cc(-c4ccc5c(c4)c4ccccc4n5-c4ccc(-c5cccc6sc7ccccc7c56)cc4)ccc32)cc1] 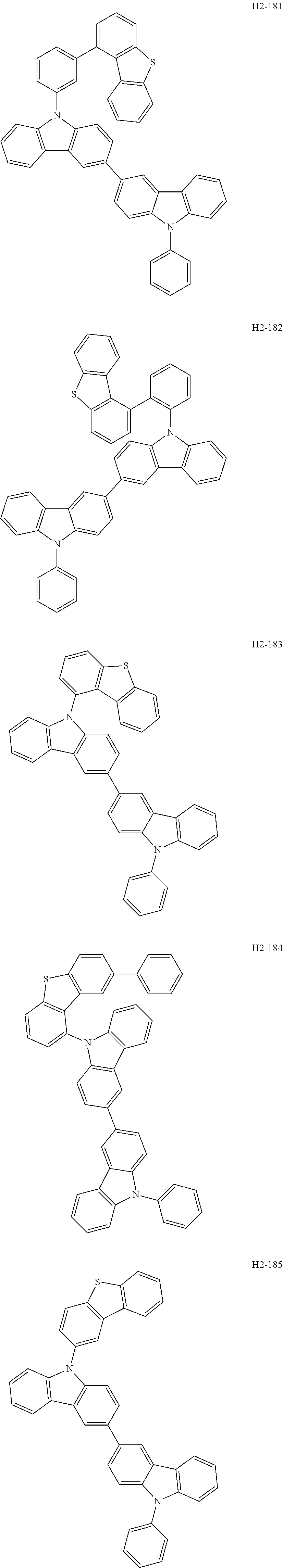 [SMILES: c1ccc(-n2c3ccccc3c3cc(-c4ccc5c(c4)c4ccccc4n5-c4cccc(-c5cccc6sc7ccccc7c56)c4)ccc32)cc1 ; c1ccc(-n2c3ccccc3c3cc(-c4ccc5c(c4)c4ccccc4n5-c4ccccc4-c4cccc5sc6ccccc6c45)ccc32)cc1 ; c1ccc(-n2c3ccccc3c3cc(-c4ccc5c(c4)c4ccccc4n5-c4cccc5sc6ccccc6c45)ccc32)cc1 ; c1ccc(-c2ccc3sc4cccc(-n5c6ccccc6c6cc(-c7ccc8c(c7)c7ccccc7n8-c7ccccc7)ccc65)c4c3c2)cc1 ; c1ccc(-n2c3ccccc3c3cc(-c4ccc5c(c4)c4ccccc4n5-c4ccc5sc6ccccc6c5c4)ccc32)cc1] 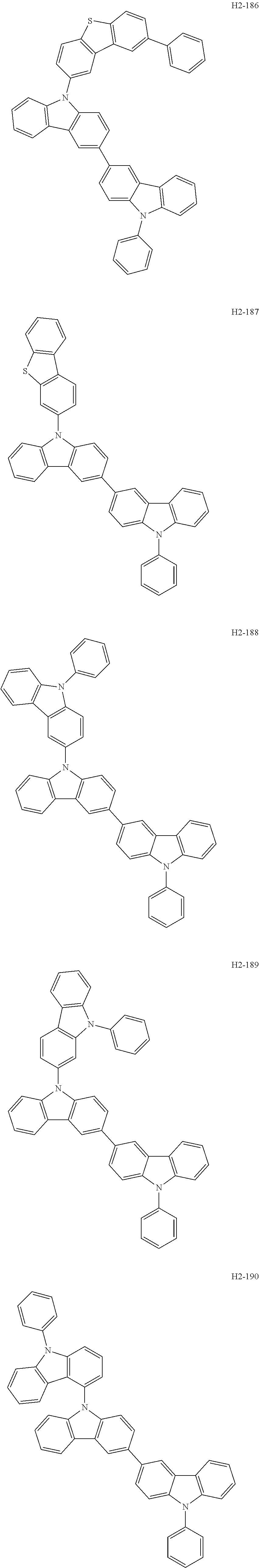 [SMILES: c1ccc(-c2ccc3sc4ccc(-n5c6ccccc6c6cc(-c7ccc8c(c7)c7ccccc7n8-c7ccccc7)ccc65)cc4c3c2)cc1 ; c1ccc(-n2c3ccccc3c3cc(-c4ccc5c(c4)c4ccccc4n5-c4ccc5c(c4)sc4ccccc45)ccc32)cc1 ; c1ccc(-n2c3ccccc3c3cc(-c4ccc5c(c4)c4ccccc4n5-c4ccc5c(c4)c4ccccc4n5-c4ccccc4)ccc32)cc1 ; c1ccc(-n2c3ccccc3c3cc(-c4ccc5c(c4)c4ccccc4n5-c4ccc5c6ccccc6n(-c6ccccc6)c5c4)ccc32)cc1 ; c1ccc(-n2c3ccccc3c3cc(-c4ccc5c(c4)c4ccccc4n5-c4cccc5c4c4ccccc4n5-c4ccccc4)ccc32)cc1] 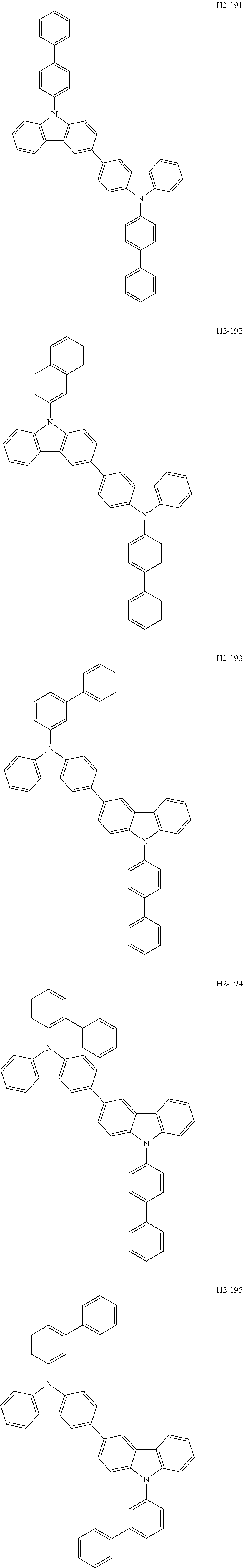 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-n3c4ccccc4c4cc(-c5ccc6c(c5)c5ccccc5n6-c5ccc6ccccc6c5)ccc43)cc2)cc1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5-c5ccccc5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1] 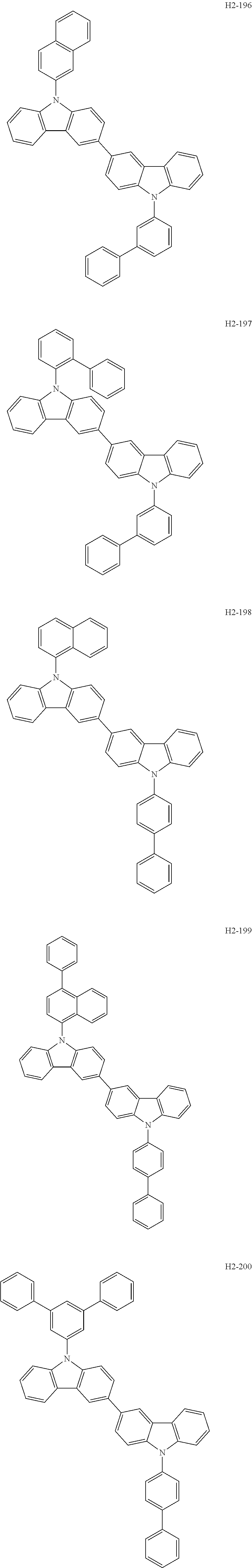 [SMILES: c1ccc(-c2cccc(-n3c4ccccc4c4cc(-c5ccc6c(c5)c5ccccc5n6-c5ccc6ccccc6c5)ccc43)c2)cc1 ; c1ccc(-c2cccc(-n3c4ccccc4c4cc(-c5ccc6c(c5)c5ccccc5n6-c5ccccc5-c5ccccc5)ccc43)c2)cc1 ; c1ccc(-c2ccc(-n3c4ccccc4c4cc(-c5ccc6c(c5)c5ccccc5n6-c5cccc6ccccc56)ccc43)cc2)cc1 ; c1ccc(-c2ccc(-n3c4ccccc4c4cc(-c5ccc6c(c5)c5ccccc5n6-c5ccc(-c6ccccc6)c6ccccc56)ccc43)cc2)cc1 ; c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1] 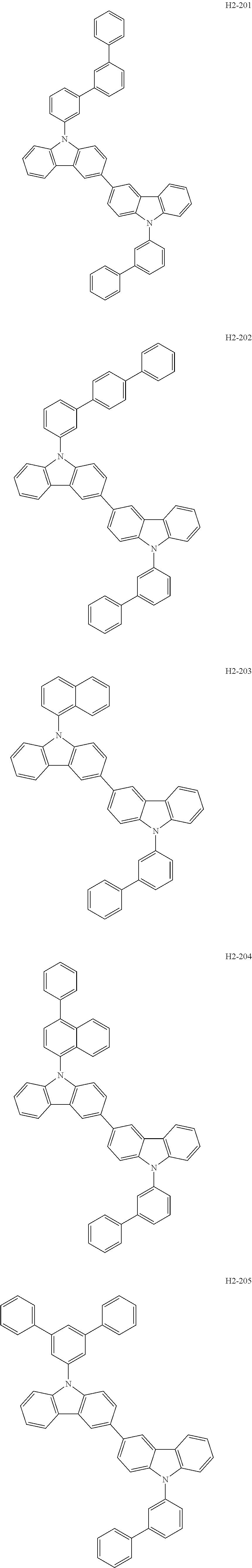 [SMILES: c1ccc(-c2cccc(-c3cccc(-n4c5ccccc5c5cc(-c6ccc7c(c6)c6ccccc6n7-c6cccc(-c7ccccc7)c6)ccc54)c3)c2)cc1 ; c1ccc(-c2ccc(-c3cccc(-n4c5ccccc5c5cc(-c6ccc7c(c6)c6ccccc6n7-c6cccc(-c7ccccc7)c6)ccc54)c3)cc2)cc1 ; c1ccc(-c2cccc(-n3c4ccccc4c4cc(-c5ccc6c(c5)c5ccccc5n6-c5cccc6ccccc56)ccc43)c2)cc1 ; c1ccc(-c2cccc(-n3c4ccccc4c4cc(-c5ccc6c(c5)c5ccccc5n6-c5ccc(-c6ccccc6)c6ccccc56)ccc43)c2)cc1 ; c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1] 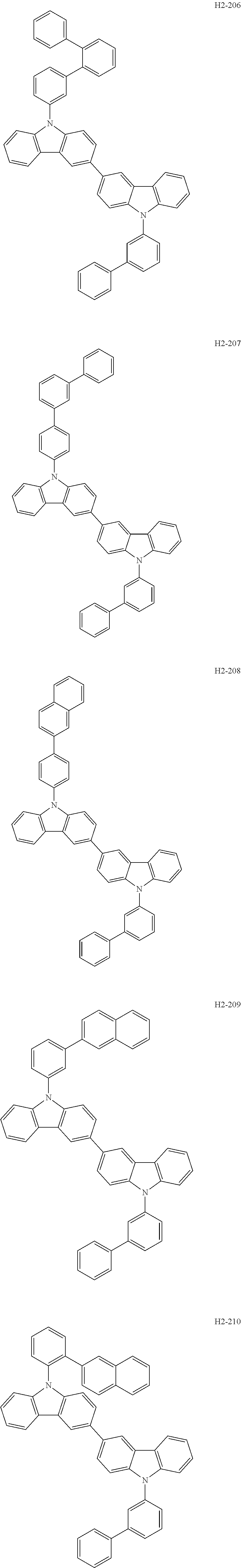 [SMILES: c1ccc(-c2cccc(-n3c4ccccc4c4cc(-c5ccc6c(c5)c5ccccc5n6-c5cccc(-c6ccccc6-c6ccccc6)c5)ccc43)c2)cc1 ; c1ccc(-c2cccc(-c3ccc(-n4c5ccccc5c5cc(-c6ccc7c(c6)c6ccccc6n7-c6cccc(-c7ccccc7)c6)ccc54)cc3)c2)cc1 ; c1ccc(-c2cccc(-n3c4ccccc4c4cc(-c5ccc6c(c5)c5ccccc5n6-c5ccc(-c6ccc7ccccc7c6)cc5)ccc43)c2)cc1 ; c1ccc(-c2cccc(-n3c4ccccc4c4cc(-c5ccc6c(c5)c5ccccc5n6-c5cccc(-c6ccc7ccccc7c6)c5)ccc43)c2)cc1 ; c1ccc(-c2cccc(-n3c4ccccc4c4cc(-c5ccc6c(c5)c5ccccc5n6-c5ccccc5-c5ccc6ccccc6c5)ccc43)c2)cc1] 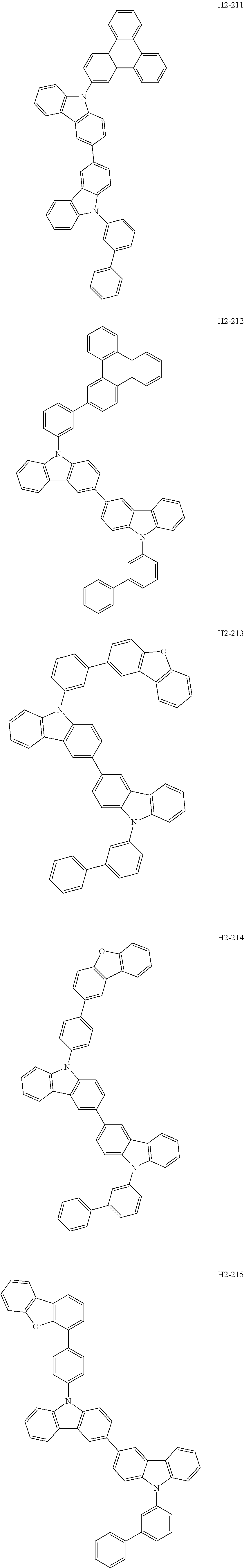 [SMILES: C1=CC2c3ccccc3-c3ccccc3C2C=C1n1c2ccccc2c2cc(-c3ccc4c(c3)c3ccccc3n4-c3cccc(-c4ccccc4)c3)ccc21 ; c1ccc(-c2cccc(-n3c4ccccc4c4cc(-c5ccc6c(c5)c5ccccc5n6-c5cccc(-c6ccc7c8ccccc8c8ccccc8c7c6)c5)ccc43)c2)cc1 ; c1ccc(-c2cccc(-n3c4ccccc4c4cc(-c5ccc6c(c5)c5ccccc5n6-c5cccc(-c6ccc7oc8ccccc8c7c6)c5)ccc43)c2)cc1 ; c1ccc(-c2cccc(-n3c4ccccc4c4cc(-c5ccc6c(c5)c5ccccc5n6-c5ccc(-c6ccc7oc8ccccc8c7c6)cc5)ccc43)c2)cc1 ; c1ccc(-c2cccc(-n3c4ccccc4c4cc(-c5ccc6c(c5)c5ccccc5n6-c5ccc(-c6cccc7c6oc6ccccc67)cc5)ccc43)c2)cc1] 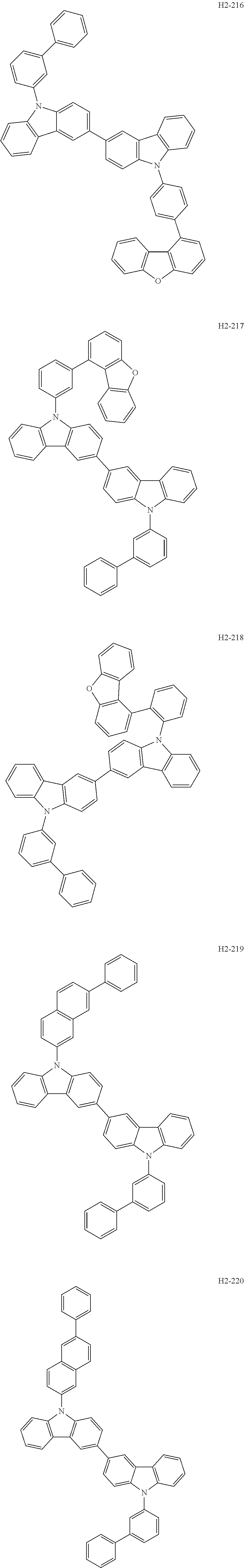 [SMILES: c1ccc(-c2cccc(-n3c4ccccc4c4cc(-c5ccc6c(c5)c5ccccc5n6-c5ccc(-c6cccc7oc8ccccc8c67)cc5)ccc43)c2)cc1 ; c1ccc(-c2cccc(-n3c4ccccc4c4cc(-c5ccc6c(c5)c5ccccc5n6-c5cccc(-c6cccc7oc8ccccc8c67)c5)ccc43)c2)cc1 ; c1ccc(-c2cccc(-n3c4ccccc4c4cc(-c5ccc6c(c5)c5ccccc5n6-c5ccccc5-c5cccc6oc7ccccc7c56)ccc43)c2)cc1 ; c1ccc(-c2cccc(-n3c4ccccc4c4cc(-c5ccc6c(c5)c5ccccc5n6-c5ccc6ccc(-c7ccccc7)cc6c5)ccc43)c2)cc1 ; c1ccc(-c2cccc(-n3c4ccccc4c4cc(-c5ccc6c(c5)c5ccccc5n6-c5ccc6cc(-c7ccccc7)ccc6c5)ccc43)c2)cc1] 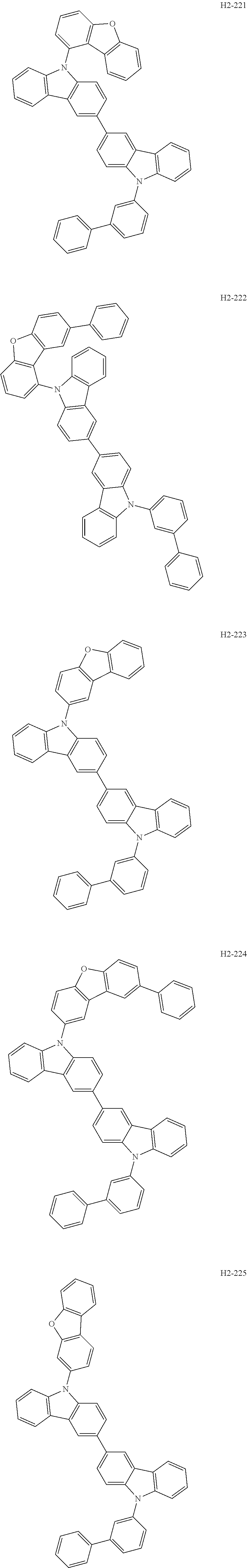 [SMILES: c1ccc(-c2cccc(-n3c4ccccc4c4cc(-c5ccc6c(c5)c5ccccc5n6-c5cccc6oc7ccccc7c56)ccc43)c2)cc1 ; c1ccc(-c2cccc(-n3c4ccccc4c4cc(-c5ccc6c(c5)c5ccccc5n6-c5cccc6oc7ccc(-c8ccccc8)cc7c56)ccc43)c2)cc1 ; c1ccc(-c2cccc(-n3c4ccccc4c4cc(-c5ccc6c(c5)c5ccccc5n6-c5ccc6oc7ccccc7c6c5)ccc43)c2)cc1 ; c1ccc(-c2cccc(-n3c4ccccc4c4cc(-c5ccc6c(c5)c5ccccc5n6-c5ccc6oc7ccc(-c8ccccc8)cc7c6c5)ccc43)c2)cc1 ; c1ccc(-c2cccc(-n3c4ccccc4c4cc(-c5ccc6c(c5)c5ccccc5n6-c5ccc6c(c5)oc5ccccc56)ccc43)c2)cc1] 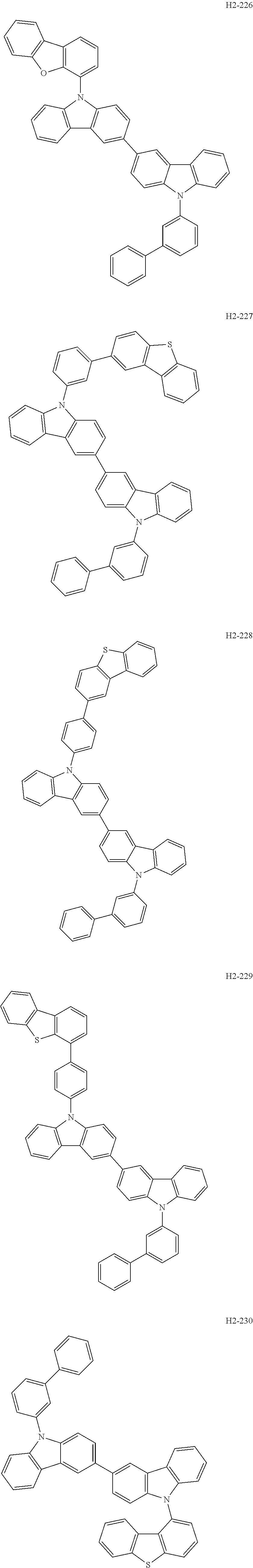 [SMILES: c1ccc(-c2cccc(-n3c4ccccc4c4cc(-c5ccc6c(c5)c5ccccc5n6-c5cccc6c5oc5ccccc56)ccc43)c2)cc1 ; c1ccc(-c2cccc(-n3c4ccccc4c4cc(-c5ccc6c(c5)c5ccccc5n6-c5cccc(-c6ccc7sc8ccccc8c7c6)c5)ccc43)c2)cc1 ; c1ccc(-c2cccc(-n3c4ccccc4c4cc(-c5ccc6c(c5)c5ccccc5n6-c5ccc(-c6ccc7sc8ccccc8c7c6)cc5)ccc43)c2)cc1 ; c1ccc(-c2cccc(-n3c4ccccc4c4cc(-c5ccc6c(c5)c5ccccc5n6-c5ccc(-c6cccc7c6sc6ccccc67)cc5)ccc43)c2)cc1 ; c1ccc(-c2cccc(-n3c4ccccc4c4cc(-c5ccc6c(c5)c5ccccc5n6-c5cccc6sc7ccccc7c56)ccc43)c2)cc1] 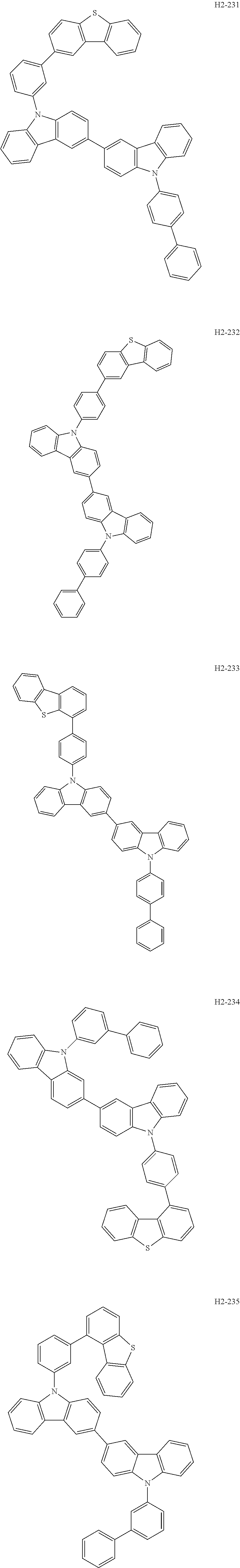 [SMILES: c1ccc(-c2ccc(-n3c4ccccc4c4cc(-c5ccc6c(c5)c5ccccc5n6-c5cccc(-c6ccc7sc8ccccc8c7c6)c5)ccc43)cc2)cc1 ; c1ccc(-c2ccc(-n3c4ccccc4c4cc(-c5ccc6c(c5)c5ccccc5n6-c5ccc(-c6ccc7sc8ccccc8c7c6)cc5)ccc43)cc2)cc1 ; c1ccc(-c2ccc(-n3c4ccccc4c4cc(-c5ccc6c(c5)c5ccccc5n6-c5ccc(-c6cccc7c6sc6ccccc67)cc5)ccc43)cc2)cc1 ; c1ccc(-c2cccc(-n3c4ccccc4c4ccc(-c5ccc6c(c5)c5ccccc5n6-c5ccc(-c6cccc7sc8ccccc8c67)cc5)cc43)c2)cc1 ; c1ccc(-c2cccc(-n3c4ccccc4c4cc(-c5ccc6c(c5)c5ccccc5n6-c5cccc(-c6cccc7sc8ccccc8c67)c5)ccc43)c2)cc1] 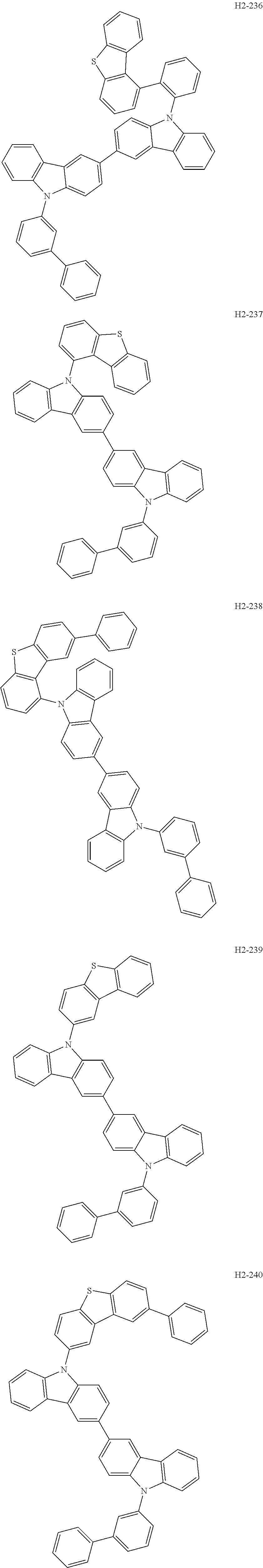 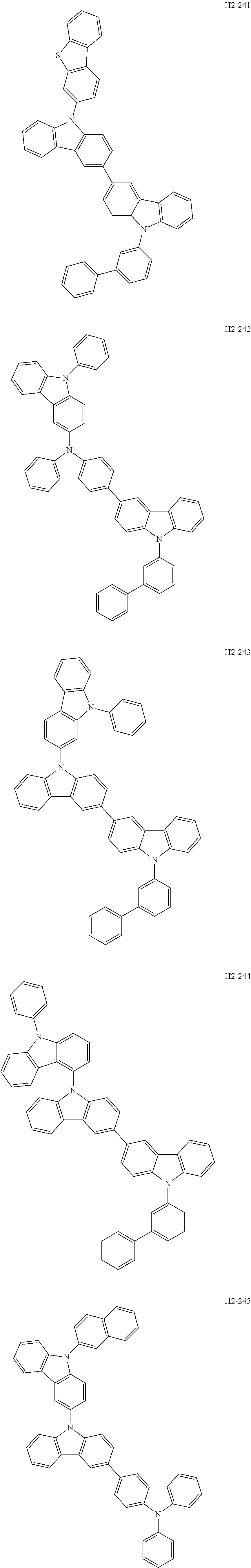 [SMILES: c1ccc(-c2cccc(-n3c4ccccc4c4cc(-c5ccc6c(c5)c5ccccc5n6-c5ccc6c(c5)sc5ccccc56)ccc43)c2)cc1 ; c1ccc(-c2cccc(-n3c4ccccc4c4cc(-c5ccc6c(c5)c5ccccc5n6-c5ccc6c(c5)c5ccccc5n6-c5ccccc5)ccc43)c2)cc1 ; c1ccc(-c2cccc(-n3c4ccccc4c4cc(-c5ccc6c(c5)c5ccccc5n6-c5ccc6c7ccccc7n(-c7ccccc7)c6c5)ccc43)c2)cc1 ; c1ccc(-c2cccc(-n3c4ccccc4c4cc(-c5ccc6c(c5)c5ccccc5n6-c5cccc6c5c5ccccc5n6-c5ccccc5)ccc43)c2)cc1 ; c1ccc(-n2c3ccccc3c3cc(-c4ccc5c(c4)c4ccccc4n5-c4ccc5c(c4)c4ccccc4n5-c4ccc5ccccc5c4)ccc32)cc1] 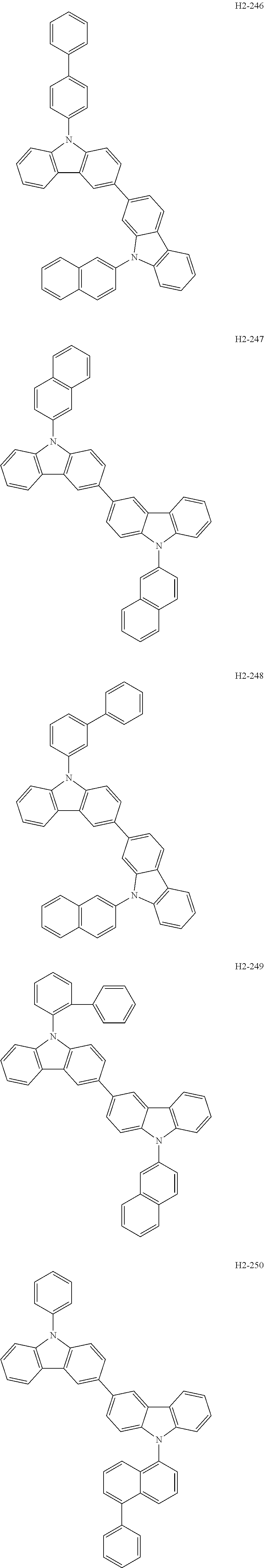 [SMILES: c1ccc(-c2ccc(-n3c4ccccc4c4cc(-c5ccc6c7ccccc7n(-c7ccc8ccccc8c7)c6c5)ccc43)cc2)cc1 ; c1ccc2cc(-n3c4ccccc4c4cc(-c5ccc6c(c5)c5ccccc5n6-c5ccc6ccccc6c5)ccc43)ccc2c1 ; c1ccc(-c2cccc(-n3c4ccccc4c4cc(-c5ccc6c7ccccc7n(-c7ccc8ccccc8c7)c6c5)ccc43)c2)cc1 ; c1ccc(-c2ccccc2-n2c3ccccc3c3cc(-c4ccc5c(c4)c4ccccc4n5-c4ccc5ccccc5c4)ccc32)cc1 ; c1ccc(-c2cccc3c(-n4c5ccccc5c5cc(-c6ccc7c(c6)c6ccccc6n7-c6ccccc6)ccc54)cccc23)cc1] 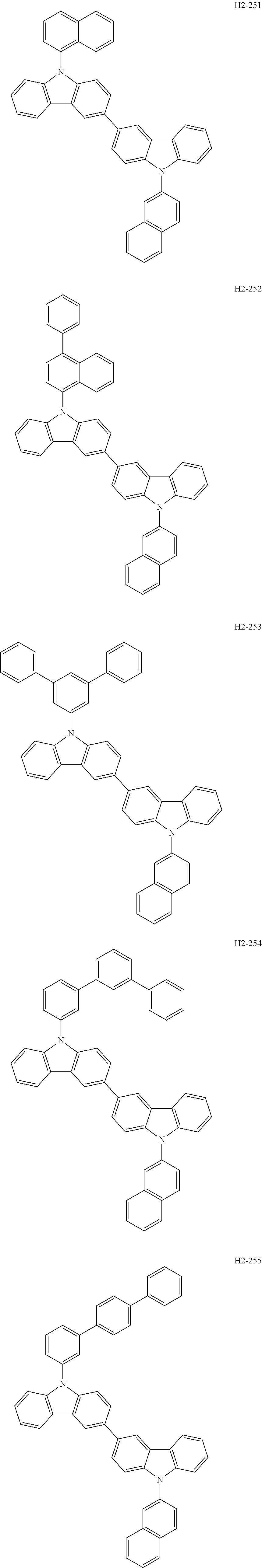 [SMILES: c1ccc2cc(-n3c4ccccc4c4cc(-c5ccc6c(c5)c5ccccc5n6-c5cccc6ccccc56)ccc43)ccc2c1 ; c1ccc(-c2ccc(-n3c4ccccc4c4cc(-c5ccc6c(c5)c5ccccc5n6-c5ccc6ccccc6c5)ccc43)c3ccccc23)cc1 ; c1ccc(-c2cc(-c3ccccc3)cc(-n3c4ccccc4c4cc(-c5ccc6c(c5)c5ccccc5n6-c5ccc6ccccc6c5)ccc43)c2)cc1 ; c1ccc(-c2cccc(-c3cccc(-n4c5ccccc5c5cc(-c6ccc7c(c6)c6ccccc6n7-c6ccc7ccccc7c6)ccc54)c3)c2)cc1 ; c1ccc(-c2ccc(-c3cccc(-n4c5ccccc5c5cc(-c6ccc7c(c6)c6ccccc6n7-c6ccc7ccccc7c6)ccc54)c3)cc2)cc1] 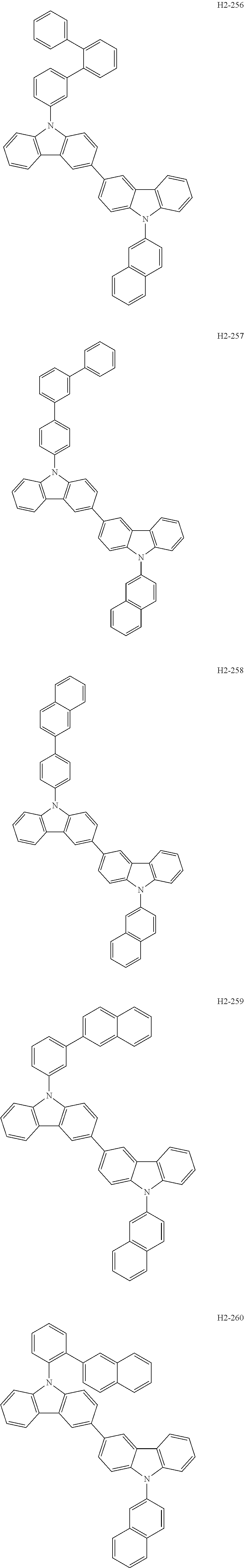 [SMILES: c1ccc(-c2ccccc2-c2cccc(-n3c4ccccc4c4cc(-c5ccc6c(c5)c5ccccc5n6-c5ccc6ccccc6c5)ccc43)c2)cc1 ; c1ccc(-c2cccc(-c3ccc(-n4c5ccccc5c5cc(-c6ccc7c(c6)c6ccccc6n7-c6ccc7ccccc7c6)ccc54)cc3)c2)cc1 ; c1ccc2cc(-c3ccc(-n4c5ccccc5c5cc(-c6ccc7c(c6)c6ccccc6n7-c6ccc7ccccc7c6)ccc54)cc3)ccc2c1 ; c1cc(-c2ccc3ccccc3c2)cc(-n2c3ccccc3c3cc(-c4ccc5c(c4)c4ccccc4n5-c4ccc5ccccc5c4)ccc32)c1 ; c1ccc(-n2c3ccccc3c3cc(-c4ccc5c(c4)c4ccccc4n5-c4ccc5ccccc5c4)ccc32)c(-c2ccc3ccccc3c2)c1] 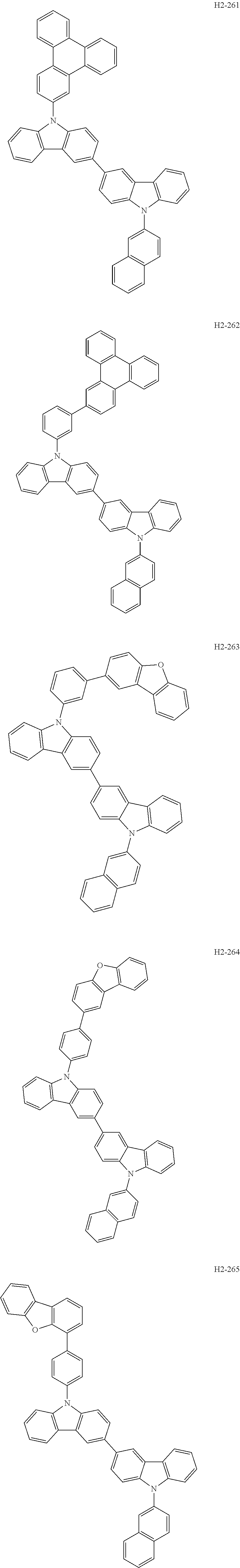 [SMILES: c1ccc2cc(-n3c4ccccc4c4cc(-c5ccc6c(c5)c5ccccc5n6-c5ccc6c7ccccc7c7ccccc7c6c5)ccc43)ccc2c1 ; c1cc(-c2ccc3c4ccccc4c4ccccc4c3c2)cc(-n2c3ccccc3c3cc(-c4ccc5c(c4)c4ccccc4n5-c4ccc5ccccc5c4)ccc32)c1 ; c1cc(-c2ccc3oc4ccccc4c3c2)cc(-n2c3ccccc3c3cc(-c4ccc5c(c4)c4ccccc4n5-c4ccc5ccccc5c4)ccc32)c1 ; c1ccc2cc(-n3c4ccccc4c4cc(-c5ccc6c(c5)c5ccccc5n6-c5ccc(-c6ccc7oc8ccccc8c7c6)cc5)ccc43)ccc2c1 ; c1ccc2cc(-n3c4ccccc4c4cc(-c5ccc6c(c5)c5ccccc5n6-c5ccc(-c6cccc7c6oc6ccccc67)cc5)ccc43)ccc2c1] 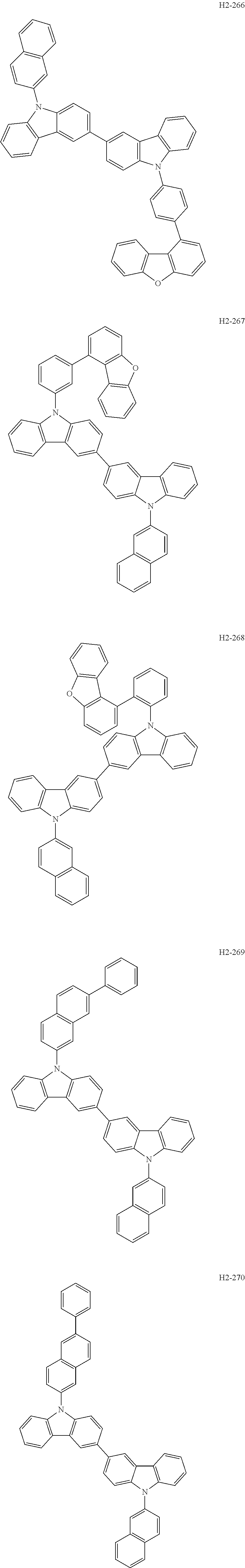 [SMILES: c1ccc2cc(-n3c4ccccc4c4cc(-c5ccc6c(c5)c5ccccc5n6-c5ccc(-c6cccc7oc8ccccc8c67)cc5)ccc43)ccc2c1 ; c1cc(-c2cccc3oc4ccccc4c23)cc(-n2c3ccccc3c3cc(-c4ccc5c(c4)c4ccccc4n5-c4ccc5ccccc5c4)ccc32)c1 ; c1ccc(-n2c3ccccc3c3cc(-c4ccc5c(c4)c4ccccc4n5-c4ccc5ccccc5c4)ccc32)c(-c2cccc3oc4ccccc4c23)c1 ; c1ccc(-c2ccc3ccc(-n4c5ccccc5c5cc(-c6ccc7c(c6)c6ccccc6n7-c6ccc7ccccc7c6)ccc54)cc3c2)cc1 ; c1ccc(-c2ccc3cc(-n4c5ccccc5c5cc(-c6ccc7c(c6)c6ccccc6n7-c6ccc7ccccc7c6)ccc54)ccc3c2)cc1] 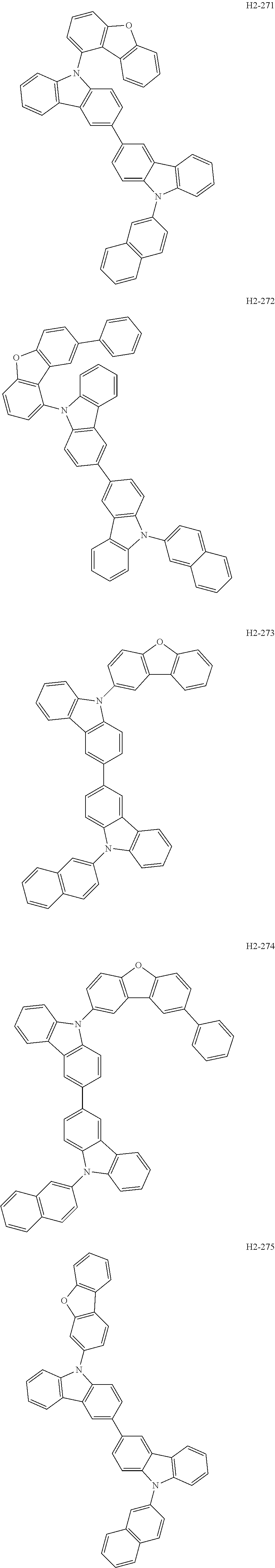 [SMILES: c1ccc2cc(-n3c4ccccc4c4cc(-c5ccc6c(c5)c5ccccc5n6-c5cccc6oc7ccccc7c56)ccc43)ccc2c1 ; c1ccc(-c2ccc3oc4cccc(-n5c6ccccc6c6cc(-c7ccc8c(c7)c7ccccc7n8-c7ccc8ccccc8c7)ccc65)c4c3c2)cc1 ; c1ccc2cc(-n3c4ccccc4c4cc(-c5ccc6c(c5)c5ccccc5n6-c5ccc6oc7ccccc7c6c5)ccc43)ccc2c1 ; c1ccc(-c2ccc3oc4ccc(-n5c6ccccc6c6cc(-c7ccc8c(c7)c7ccccc7n8-c7ccc8ccccc8c7)ccc65)cc4c3c2)cc1 ; c1ccc2cc(-n3c4ccccc4c4cc(-c5ccc6c(c5)c5ccccc5n6-c5ccc6c(c5)oc5ccccc56)ccc43)ccc2c1] 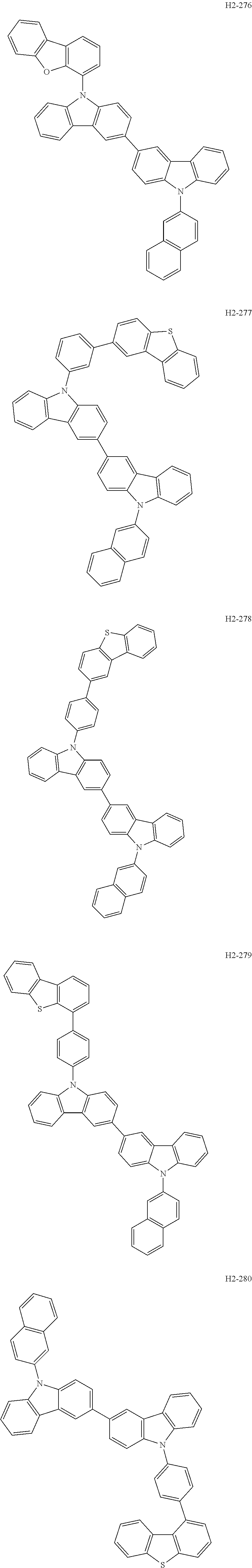 [SMILES: c1ccc2cc(-n3c4ccccc4c4cc(-c5ccc6c(c5)c5ccccc5n6-c5cccc6c5oc5ccccc56)ccc43)ccc2c1 ; c1cc(-c2ccc3sc4ccccc4c3c2)cc(-n2c3ccccc3c3cc(-c4ccc5c(c4)c4ccccc4n5-c4ccc5ccccc5c4)ccc32)c1 ; c1ccc2cc(-n3c4ccccc4c4cc(-c5ccc6c(c5)c5ccccc5n6-c5ccc(-c6ccc7sc8ccccc8c7c6)cc5)ccc43)ccc2c1 ; c1ccc2cc(-n3c4ccccc4c4cc(-c5ccc6c(c5)c5ccccc5n6-c5ccc(-c6cccc7c6sc6ccccc67)cc5)ccc43)ccc2c1 ; c1ccc2cc(-n3c4ccccc4c4cc(-c5ccc6c(c5)c5ccccc5n6-c5ccc(-c6cccc7sc8ccccc8c67)cc5)ccc43)ccc2c1] 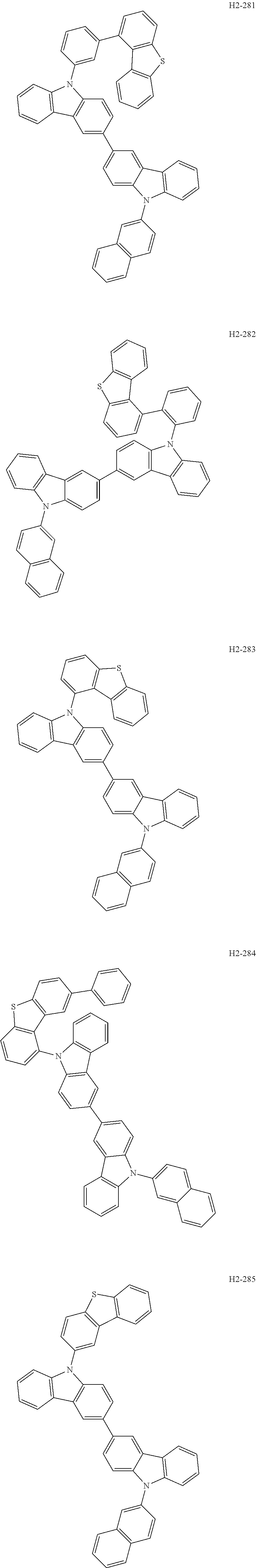 [SMILES: c1cc(-c2cccc3sc4ccccc4c23)cc(-n2c3ccccc3c3cc(-c4ccc5c(c4)c4ccccc4n5-c4ccc5ccccc5c4)ccc32)c1 ; c1ccc(-n2c3ccccc3c3cc(-c4ccc5c(c4)c4ccccc4n5-c4ccc5ccccc5c4)ccc32)c(-c2cccc3sc4ccccc4c23)c1 ; c1ccc2cc(-n3c4ccccc4c4cc(-c5ccc6c(c5)c5ccccc5n6-c5cccc6sc7ccccc7c56)ccc43)ccc2c1 ; c1ccc(-c2ccc3sc4cccc(-n5c6ccccc6c6cc(-c7ccc8c(c7)c7ccccc7n8-c7ccc8ccccc8c7)ccc65)c4c3c2)cc1 ; c1ccc2cc(-n3c4ccccc4c4cc(-c5ccc6c(c5)c5ccccc5n6-c5ccc6sc7ccccc7c6c5)ccc43)ccc2c1] 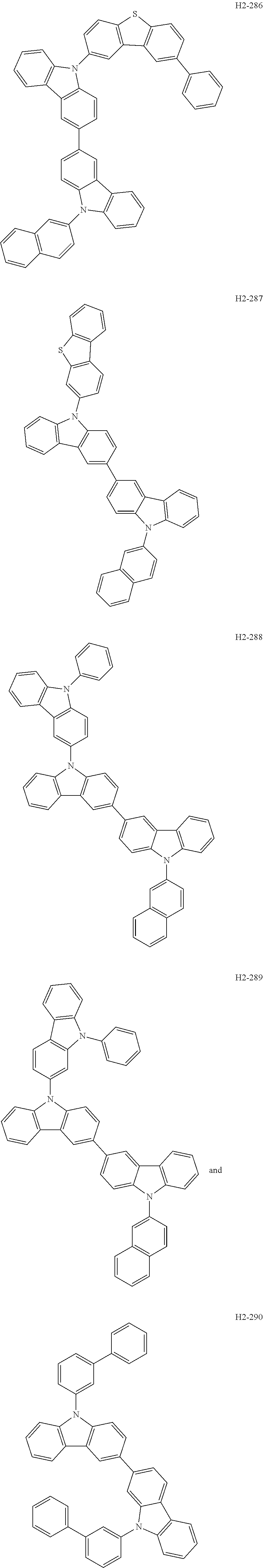 [SMILES: c1ccc(-c2ccc3sc4ccc(-n5c6ccccc6c6cc(-c7ccc8c(c7)c7ccccc7n8-c7ccc8ccccc8c7)ccc65)cc4c3c2)cc1 ; c1ccc2cc(-n3c4ccccc4c4cc(-c5ccc6c(c5)c5ccccc5n6-c5ccc6c(c5)sc5ccccc56)ccc43)ccc2c1 ; c1ccc(-n2c3ccccc3c3cc(-n4c5ccccc5c5cc(-c6ccc7c(c6)c6ccccc6n7-c6ccc7ccccc7c6)ccc54)ccc32)cc1 ; c1ccc(-n2c3ccccc3c3ccc(-n4c5ccccc5c5cc(-c6ccc7c(c6)c6ccccc6n7-c6ccc7ccccc7c6)ccc54)cc32)cc1 ; c1ccc(-c2cccc(-n3c4ccccc4c4cc(-c5ccc6c7ccccc7n(-c7cccc(-c8ccccc8)c7)c6c5)ccc43)c2)cc1]
In the above compounds, Dn means that n hydrogens are substituted with deuterium.

15. An organic electroluminescent compound represented by the following Formula 11: 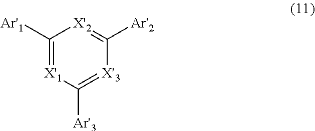
Wherein,
X′1 to X′₃ each independently represent N or CR′ₐ; provided that at least two of X′1 to X′₃ represent N;
R′ₐ represents hydrogen or deuterium;
Ar′₁ to Ar′₃ each independently represent (C6-C30)aryl unsubstituted or substituted with at least one of deuterium and (C6-C30)aryl, or are represented by the following Formula A; provided that at least one of Ar′₁ to Ar′₃ is(are) represented by the following Formula A; 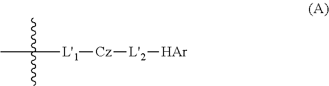
Wherein,
L′₁ represents a single bond, or a substituted or unsubstituted (C6-C12)arylene;
Cz represents a substituted or unsubstituted carbazolylene;
L′₂ represents a substituted or unsubstituted (C6-C12)arylene; and
HAr represents a substituted or unsubstituted dibenzofuranyl, a substituted or unsubstituted carbazolyl, or a substituted or unsubstituted dibenzothiophenyl.

16. The organic electroluminescent compound according to claim 15, wherein Formula A is represented by the following Formula A-1: 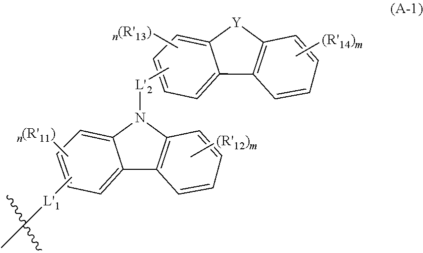
wherein,
L′₁ and L′₂ are as defined in claim 15;
Y represents —O—, —S—, or —N(R′₁)—;
R′₁ represents a substituted or unsubstituted (C6-C12)aryl, or a substituted or unsubstituted dibenzofuranyl, a substituted or unsubstituted carbazolyl, or a substituted or unsubstituted dibenzothiophenyl;
R′₁₁ to R′₁₄ each independently represent hydrogen, deuterium, a substituted or unsubstituted (C6-C12)aryl, or a substituted or unsubstituted dibenzofuranyl, a substituted or unsubstituted carbazolyl, or a substituted or unsubstituted dibenzothiophenyl;
n is an integer of 1 to 3, m is an integer of 1 to 4;
when m and n are 2 or more, each of R′₁₁ to R′₁₄ may be the same or different from each other.

17. The organic electroluminescent compound according to claim 15, wherein the compound represented by the Formula 11 is selected from the following compounds:

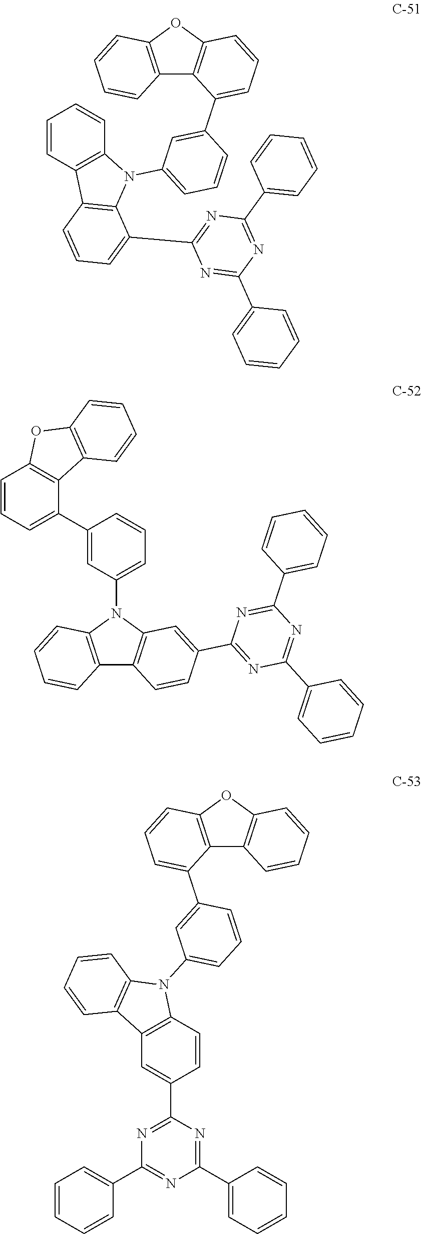 [SMILES: c1ccc(-c2nc(-c3ccccc3)nc(-c3cccc4c5ccccc5n(-c5cccc(-c6cccc7oc8ccccc8c67)c5)c34)n2)cc1 ; c1ccc(-c2nc(-c3ccccc3)nc(-c3ccc4c5ccccc5n(-c5cccc(-c6cccc7oc8ccccc8c67)c5)c4c3)n2)cc1 ; c1ccc(-c2nc(-c3ccccc3)nc(-c3ccc4c(c3)c3ccccc3n4-c3cccc(-c4cccc5oc6ccccc6c45)c3)n2)cc1]

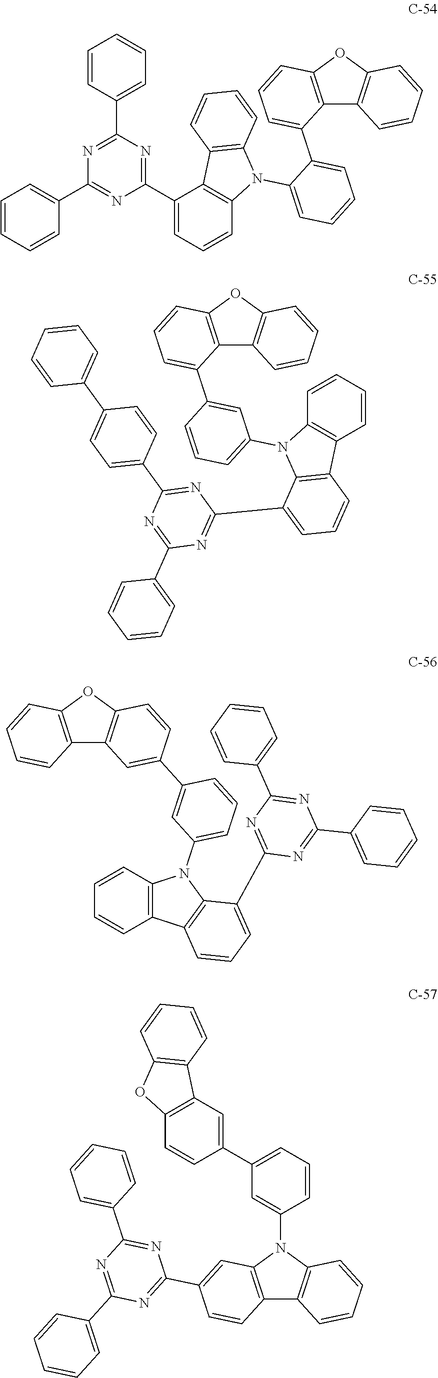 [SMILES: c1ccc(-c2nc(-c3ccccc3)nc(-c3cccc4c3c3ccccc3n4-c3ccccc3-c3cccc4oc5ccccc5c34)n2)cc1 ; c1ccc(-c2ccc(-c3nc(-c4ccccc4)nc(-c4cccc5c6ccccc6n(-c6cccc(-c7cccc8oc9ccccc9c78)c6)c45)n3)cc2)cc1 ; c1ccc(-c2nc(-c3ccccc3)nc(-c3cccc4c5ccccc5n(-c5cccc(-c6ccc7oc8ccccc8c7c6)c5)c34)n2)cc1 ; c1ccc(-c2nc(-c3ccccc3)nc(-c3ccc4c5ccccc5n(-c5cccc(-c6ccc7oc8ccccc8c7c6)c5)c4c3)n2)cc1]

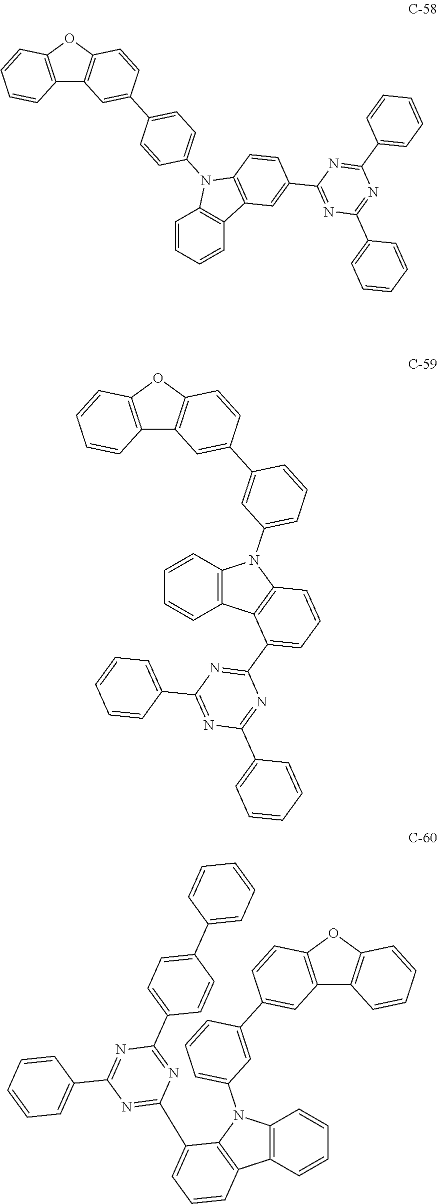 [SMILES: c1ccc(-c2nc(-c3ccccc3)nc(-c3ccc4c(c3)c3ccccc3n4-c3ccc(-c4ccc5oc6ccccc6c5c4)cc3)n2)cc1 ; c1ccc(-c2nc(-c3ccccc3)nc(-c3cccc4c3c3ccccc3n4-c3cccc(-c4ccc5oc6ccccc6c5c4)c3)n2)cc1 ; c1ccc(-c2ccc(-c3nc(-c4ccccc4)nc(-c4cccc5c6ccccc6n(-c6cccc(-c7ccc8oc9ccccc9c8c7)c6)c45)n3)cc2)cc1]

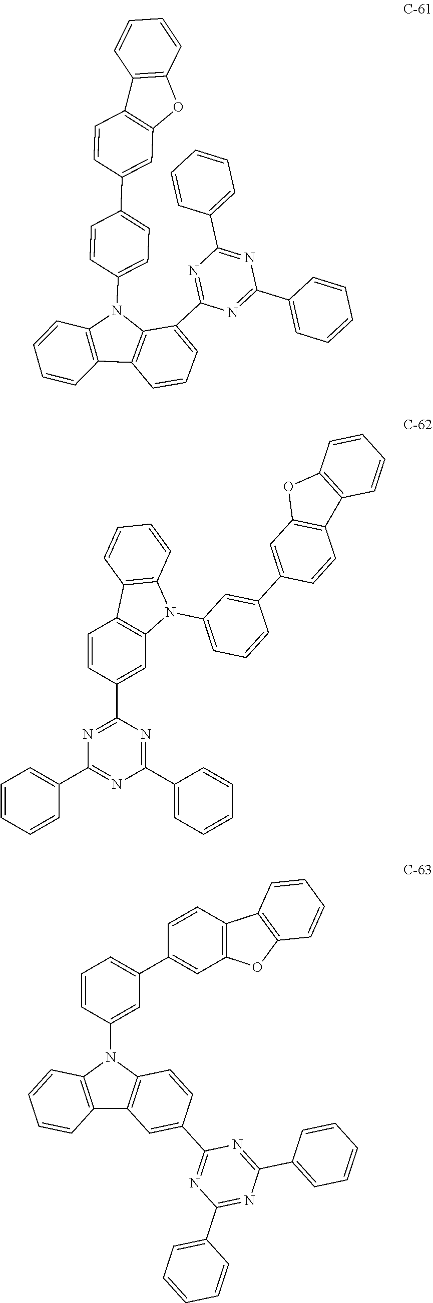 [SMILES: c1ccc(-c2nc(-c3ccccc3)nc(-c3cccc4c5ccccc5n(-c5ccc(-c6ccc7c(c6)oc6ccccc67)cc5)c34)n2)cc1 ; c1ccc(-c2nc(-c3ccccc3)nc(-c3ccc4c5ccccc5n(-c5cccc(-c6ccc7c(c6)oc6ccccc67)c5)c4c3)n2)cc1 ; c1ccc(-c2nc(-c3ccccc3)nc(-c3ccc4c(c3)c3ccccc3n4-c3cccc(-c4ccc5c(c4)oc4ccccc45)c3)n2)cc1]

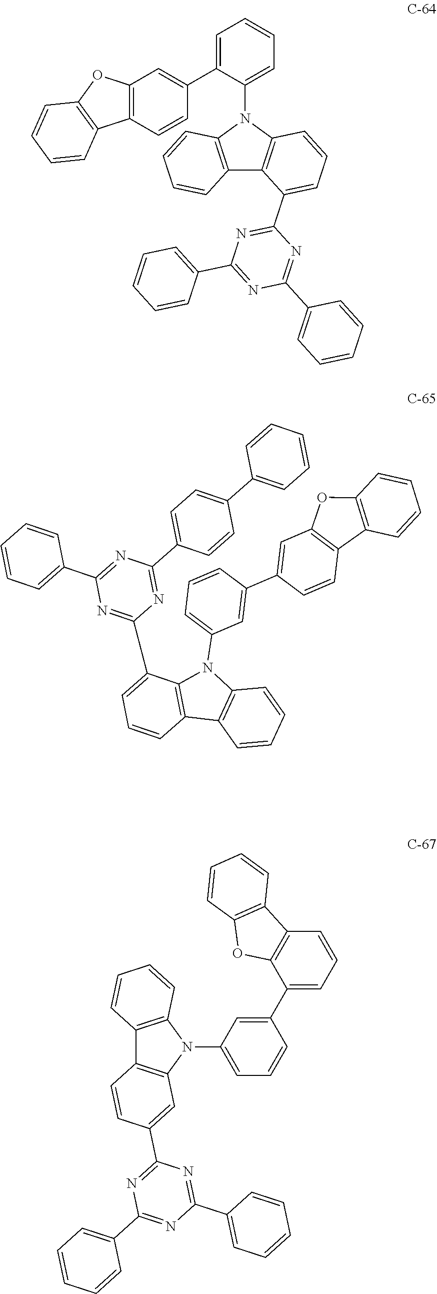 [SMILES: c1ccc(-c2nc(-c3ccccc3)nc(-c3cccc4c3c3ccccc3n4-c3ccccc3-c3ccc4c(c3)oc3ccccc34)n2)cc1 ; c1ccc(-c2ccc(-c3nc(-c4ccccc4)nc(-c4cccc5c6ccccc6n(-c6cccc(-c7ccc8c(c7)oc7ccccc78)c6)c45)n3)cc2)cc1 ; c1ccc(-c2nc(-c3ccccc3)nc(-c3ccc4c5ccccc5n(-c5cccc(-c6cccc7c6oc6ccccc67)c5)c4c3)n2)cc1]

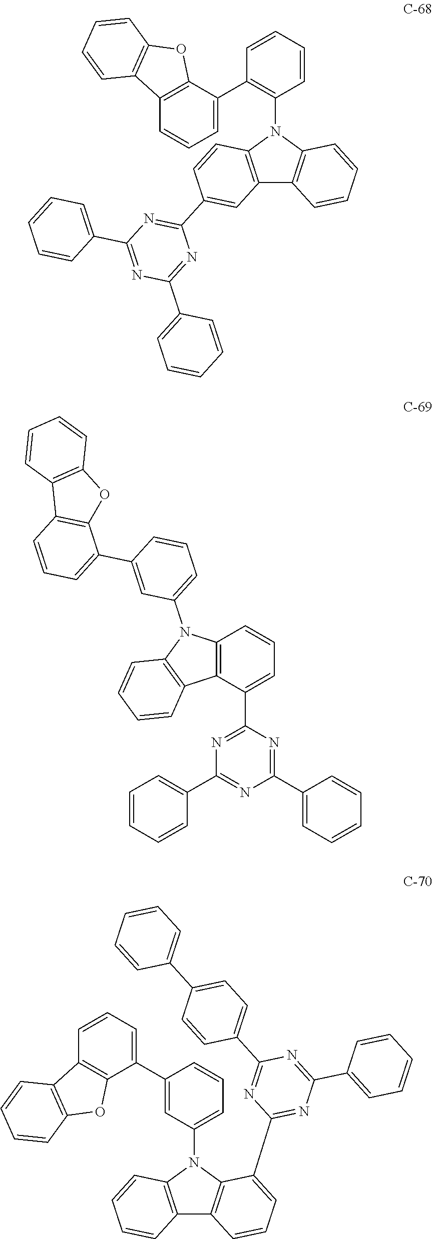 [SMILES: c1ccc(-c2nc(-c3ccccc3)nc(-c3ccc4c(c3)c3ccccc3n4-c3ccccc3-c3cccc4c3oc3ccccc34)n2)cc1 ; c1ccc(-c2nc(-c3ccccc3)nc(-c3cccc4c3c3ccccc3n4-c3cccc(-c4cccc5c4oc4ccccc45)c3)n2)cc1 ; c1ccc(-c2ccc(-c3nc(-c4ccccc4)nc(-c4cccc5c6ccccc6n(-c6cccc(-c7cccc8c7oc7ccccc78)c6)c45)n3)cc2)cc1]

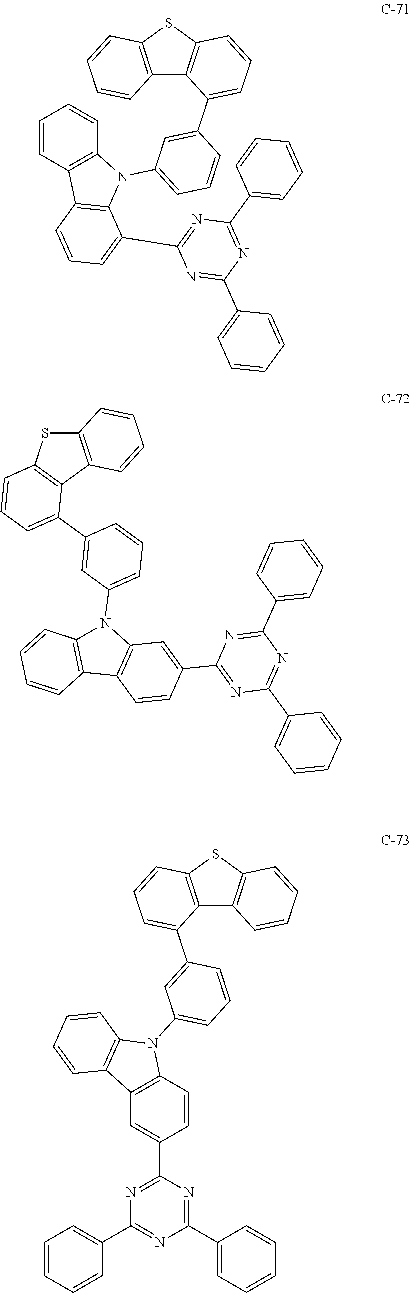 [SMILES: c1ccc(-c2nc(-c3ccccc3)nc(-c3cccc4c5ccccc5n(-c5cccc(-c6cccc7sc8ccccc8c67)c5)c34)n2)cc1 ; c1ccc(-c2nc(-c3ccccc3)nc(-c3ccc4c5ccccc5n(-c5cccc(-c6cccc7sc8ccccc8c67)c5)c4c3)n2)cc1 ; c1ccc(-c2nc(-c3ccccc3)nc(-c3ccc4c(c3)c3ccccc3n4-c3cccc(-c4cccc5sc6ccccc6c45)c3)n2)cc1]

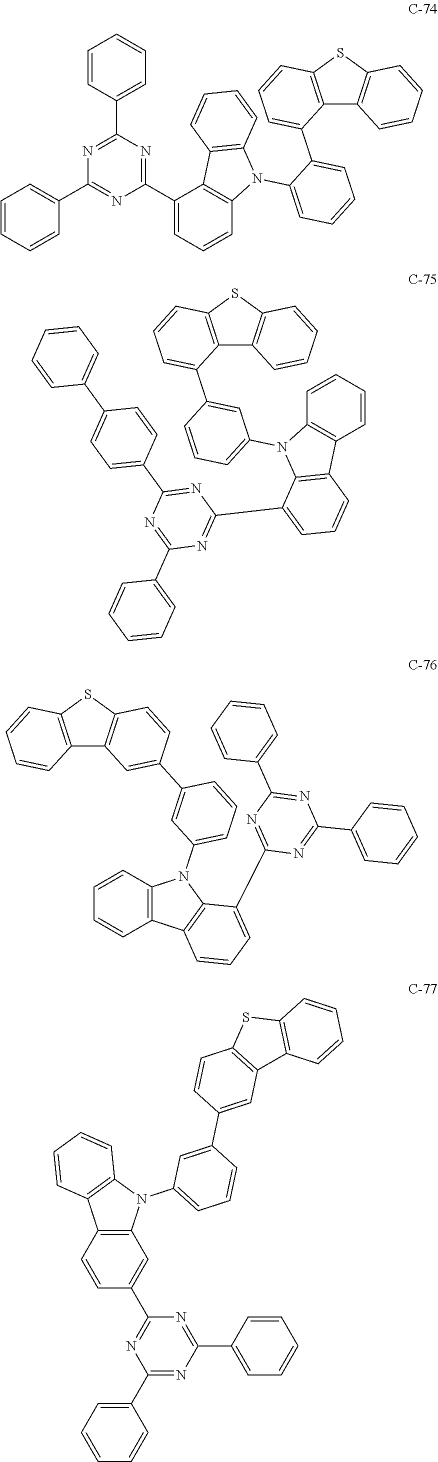 [SMILES: c1ccc(-c2nc(-c3ccccc3)nc(-c3cccc4c3c3ccccc3n4-c3ccccc3-c3cccc4sc5ccccc5c34)n2)cc1 ; c1ccc(-c2ccc(-c3nc(-c4ccccc4)nc(-c4cccc5c6ccccc6n(-c6cccc(-c7cccc8sc9ccccc9c78)c6)c45)n3)cc2)cc1 ; c1ccc(-c2nc(-c3ccccc3)nc(-c3cccc4c5ccccc5n(-c5cccc(-c6ccc7sc8ccccc8c7c6)c5)c34)n2)cc1 ; c1ccc(-c2nc(-c3ccccc3)nc(-c3ccc4c5ccccc5n(-c5cccc(-c6ccc7sc8ccccc8c7c6)c5)c4c3)n2)cc1]

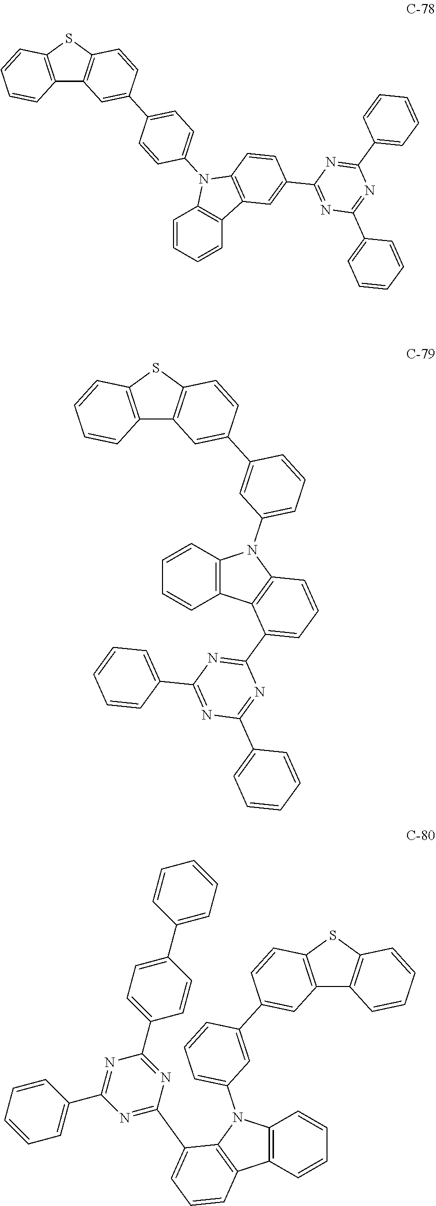 [SMILES: c1ccc(-c2nc(-c3ccccc3)nc(-c3ccc4c(c3)c3ccccc3n4-c3ccc(-c4ccc5sc6ccccc6c5c4)cc3)n2)cc1 ; c1ccc(-c2nc(-c3ccccc3)nc(-c3cccc4c3c3ccccc3n4-c3cccc(-c4ccc5sc6ccccc6c5c4)c3)n2)cc1 ; c1ccc(-c2ccc(-c3nc(-c4ccccc4)nc(-c4cccc5c6ccccc6n(-c6cccc(-c7ccc8sc9ccccc9c8c7)c6)c45)n3)cc2)cc1]

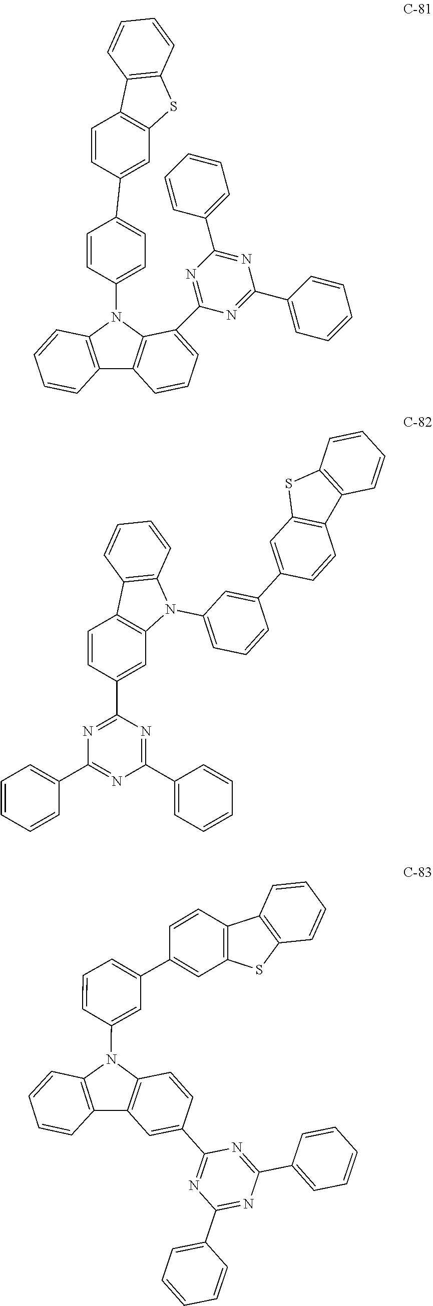 [SMILES: c1ccc(-c2nc(-c3ccccc3)nc(-c3cccc4c5ccccc5n(-c5ccc(-c6ccc7c(c6)sc6ccccc67)cc5)c34)n2)cc1 ; c1ccc(-c2nc(-c3ccccc3)nc(-c3ccc4c5ccccc5n(-c5cccc(-c6ccc7c(c6)sc6ccccc67)c5)c4c3)n2)cc1 ; c1ccc(-c2nc(-c3ccccc3)nc(-c3ccc4c(c3)c3ccccc3n4-c3cccc(-c4ccc5c(c4)sc4ccccc45)c3)n2)cc1]

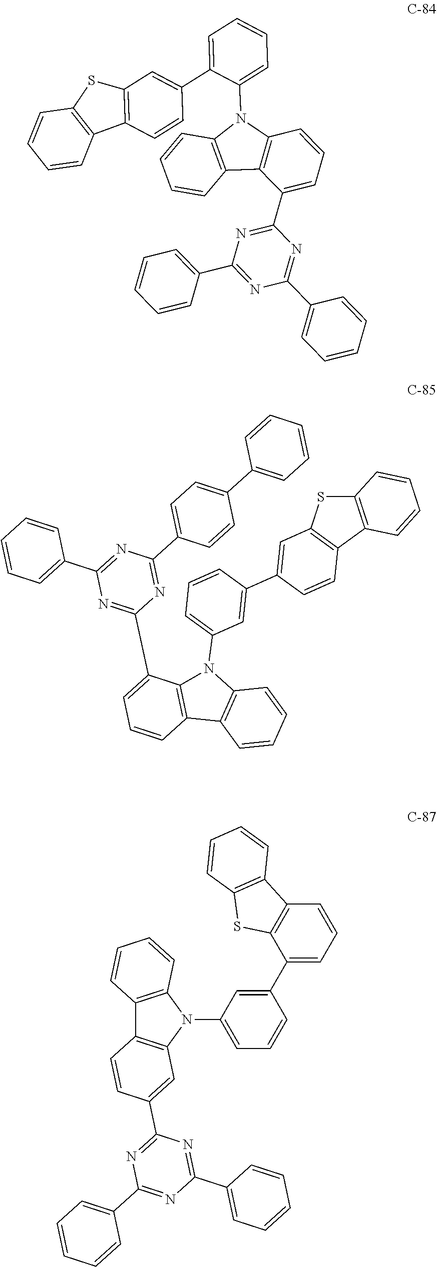 [SMILES: c1ccc(-c2nc(-c3ccccc3)nc(-c3cccc4c3c3ccccc3n4-c3ccccc3-c3ccc4c(c3)sc3ccccc34)n2)cc1 ; c1ccc(-c2ccc(-c3nc(-c4ccccc4)nc(-c4cccc5c6ccccc6n(-c6cccc(-c7ccc8c(c7)sc7ccccc78)c6)c45)n3)cc2)cc1 ; c1ccc(-c2nc(-c3ccccc3)nc(-c3ccc4c5ccccc5n(-c5cccc(-c6cccc7c6sc6ccccc67)c5)c4c3)n2)cc1]

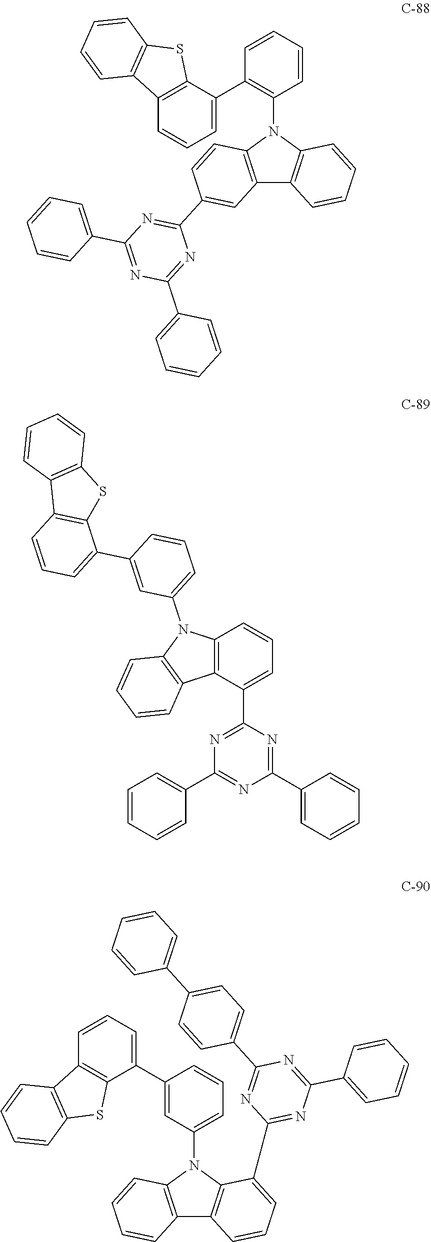 [SMILES: c1ccc(-c2nc(-c3ccccc3)nc(-c3ccc4c(c3)c3ccccc3n4-c3ccccc3-c3cccc4c3sc3ccccc34)n2)cc1 ; c1ccc(-c2nc(-c3ccccc3)nc(-c3cccc4c3c3ccccc3n4-c3cccc(-c4cccc5c4sc4ccccc45)c3)n2)cc1 ; c1ccc(-c2ccc(-c3nc(-c4ccccc4)nc(-c4cccc5c6ccccc6n(-c6cccc(-c7cccc8c7sc7ccccc78)c6)c45)n3)cc2)cc1]

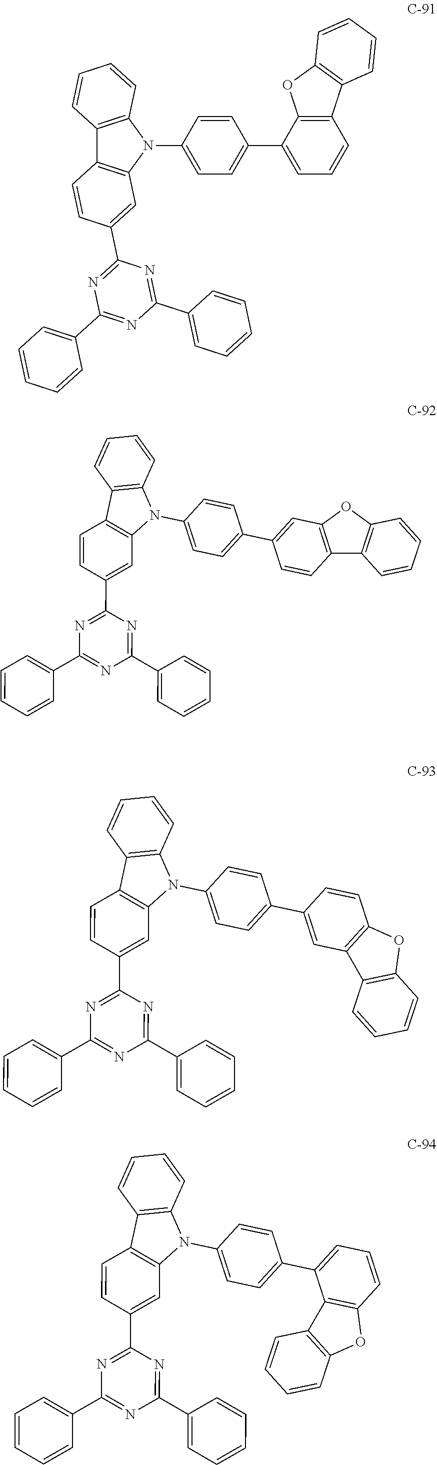 [SMILES: c1ccc(-c2nc(-c3ccccc3)nc(-c3ccc4c5ccccc5n(-c5ccc(-c6cccc7c6oc6ccccc67)cc5)c4c3)n2)cc1 ; c1ccc(-c2nc(-c3ccccc3)nc(-c3ccc4c5ccccc5n(-c5ccc(-c6ccc7c(c6)oc6ccccc67)cc5)c4c3)n2)cc1 ; c1ccc(-c2nc(-c3ccccc3)nc(-c3ccc4c5ccccc5n(-c5ccc(-c6ccc7oc8ccccc8c7c6)cc5)c4c3)n2)cc1 ; c1ccc(-c2nc(-c3ccccc3)nc(-c3ccc4c5ccccc5n(-c5ccc(-c6cccc7oc8ccccc8c67)cc5)c4c3)n2)cc1]

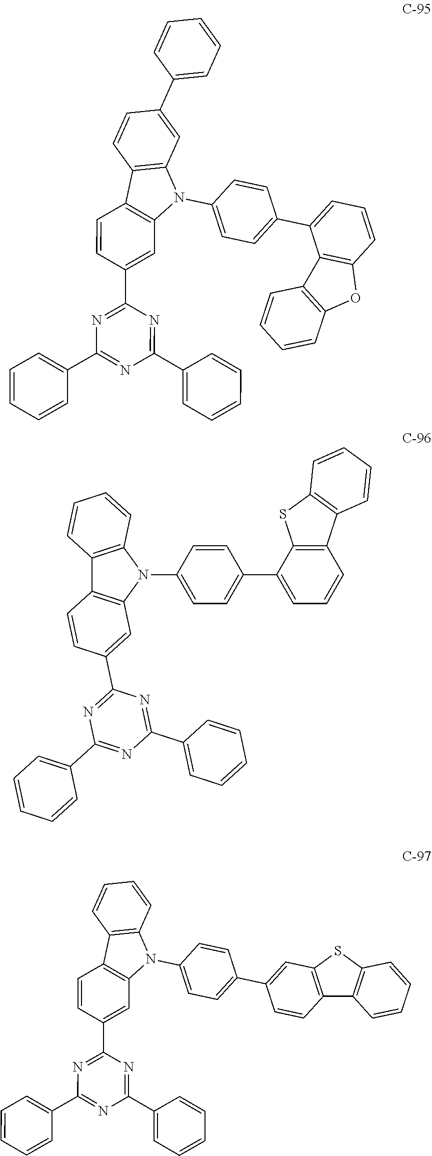 [SMILES: c1ccc(-c2ccc3c4ccc(-c5nc(-c6ccccc6)nc(-c6ccccc6)n5)cc4n(-c4ccc(-c5cccc6oc7ccccc7c56)cc4)c3c2)cc1 ; c1ccc(-c2nc(-c3ccccc3)nc(-c3ccc4c5ccccc5n(-c5ccc(-c6cccc7c6sc6ccccc67)cc5)c4c3)n2)cc1 ; c1ccc(-c2nc(-c3ccccc3)nc(-c3ccc4c5ccccc5n(-c5ccc(-c6ccc7c(c6)sc6ccccc67)cc5)c4c3)n2)cc1]

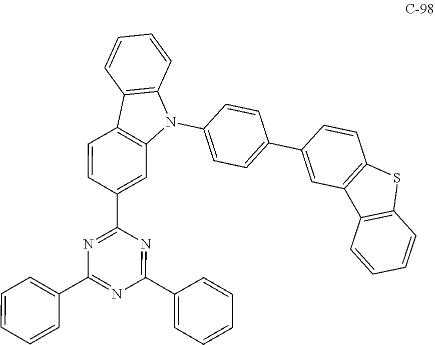 [SMILES: c1ccc(-c2nc(-c3ccccc3)nc(-c3ccc4c5ccccc5n(-c5ccc(-c6ccc7sc8ccccc8c7c6)cc5)c4c3)n2)cc1]

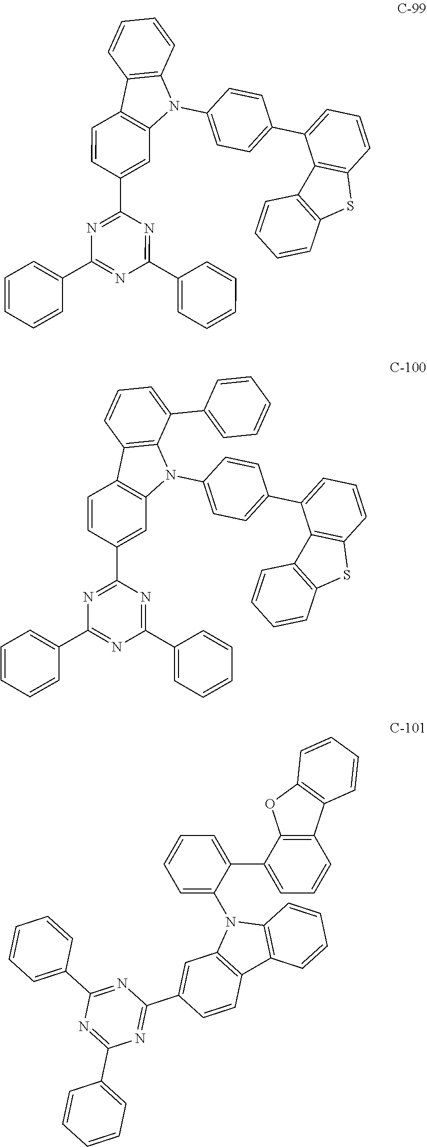 [SMILES: c1ccc(-c2nc(-c3ccccc3)nc(-c3ccc4c5ccccc5n(-c5ccc(-c6cccc7sc8ccccc8c67)cc5)c4c3)n2)cc1 ; c1ccc(-c2nc(-c3ccccc3)nc(-c3ccc4c5cccc(-c6ccccc6)c5n(-c5ccc(-c6cccc7sc8ccccc8c67)cc5)c4c3)n2)cc1 ; c1ccc(-c2nc(-c3ccccc3)nc(-c3ccc4c5ccccc5n(-c5ccccc5-c5cccc6c5oc5ccccc56)c4c3)n2)cc1]

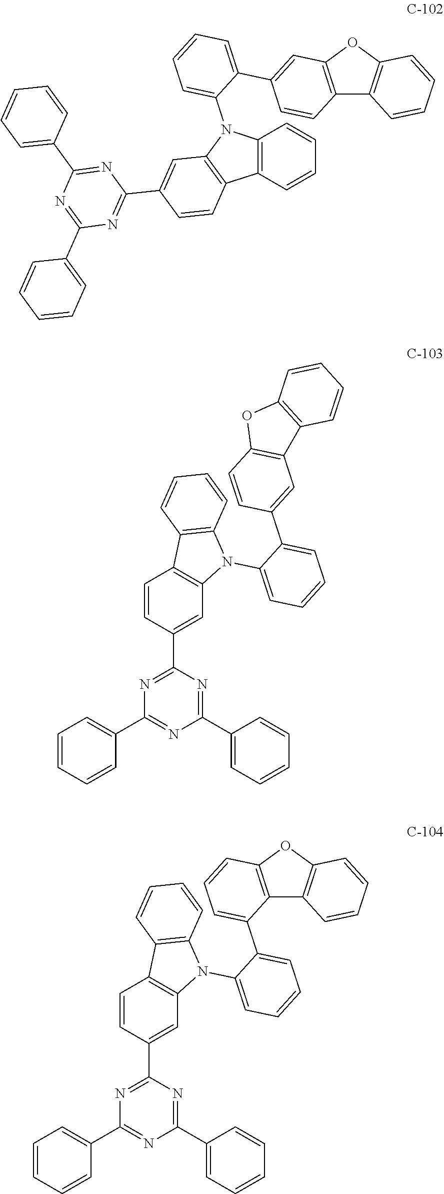 [SMILES: c1ccc(-c2nc(-c3ccccc3)nc(-c3ccc4c5ccccc5n(-c5ccccc5-c5ccc6c(c5)oc5ccccc56)c4c3)n2)cc1 ; c1ccc(-c2nc(-c3ccccc3)nc(-c3ccc4c5ccccc5n(-c5ccccc5-c5ccc6oc7ccccc7c6c5)c4c3)n2)cc1 ; c1ccc(-c2nc(-c3ccccc3)nc(-c3ccc4c5ccccc5n(-c5ccccc5-c5cccc6oc7ccccc7c56)c4c3)n2)cc1]

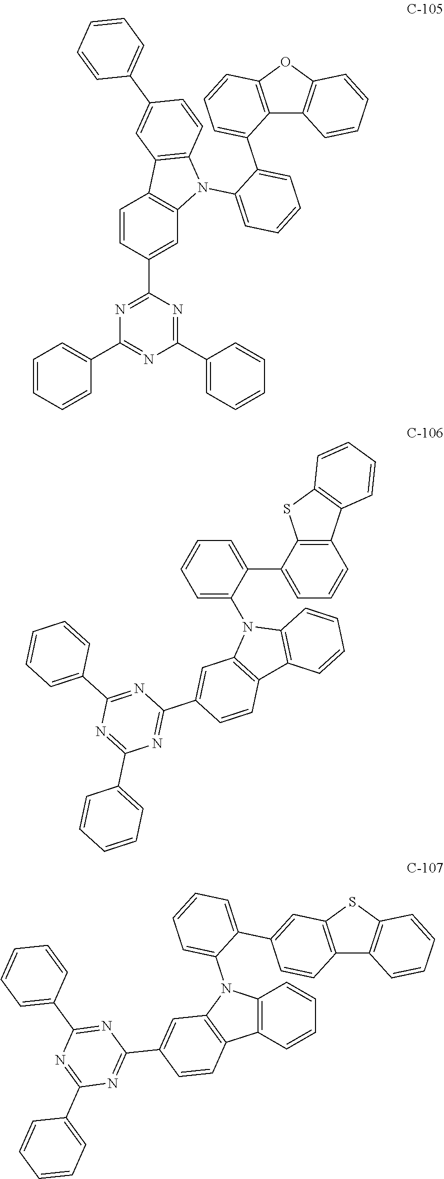 [SMILES: c1ccc(-c2ccc3c(c2)c2ccc(-c4nc(-c5ccccc5)nc(-c5ccccc5)n4)cc2n3-c2ccccc2-c2cccc3oc4ccccc4c23)cc1 ; c1ccc(-c2nc(-c3ccccc3)nc(-c3ccc4c5ccccc5n(-c5ccccc5-c5cccc6c5sc5ccccc56)c4c3)n2)cc1 ; c1ccc(-c2nc(-c3ccccc3)nc(-c3ccc4c5ccccc5n(-c5ccccc5-c5ccc6c(c5)sc5ccccc56)c4c3)n2)cc1]

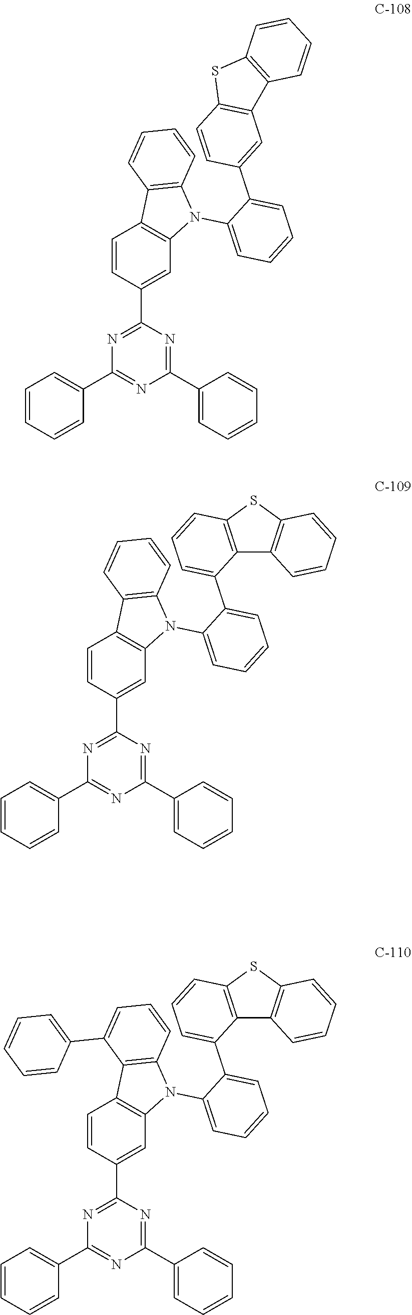 [SMILES: c1ccc(-c2nc(-c3ccccc3)nc(-c3ccc4c5ccccc5n(-c5ccccc5-c5ccc6sc7ccccc7c6c5)c4c3)n2)cc1 ; c1ccc(-c2nc(-c3ccccc3)nc(-c3ccc4c5ccccc5n(-c5ccccc5-c5cccc6sc7ccccc7c56)c4c3)n2)cc1 ; c1ccc(-c2nc(-c3ccccc3)nc(-c3ccc4c5c(-c6ccccc6)cccc5n(-c5ccccc5-c5cccc6sc7ccccc7c56)c4c3)n2)cc1]

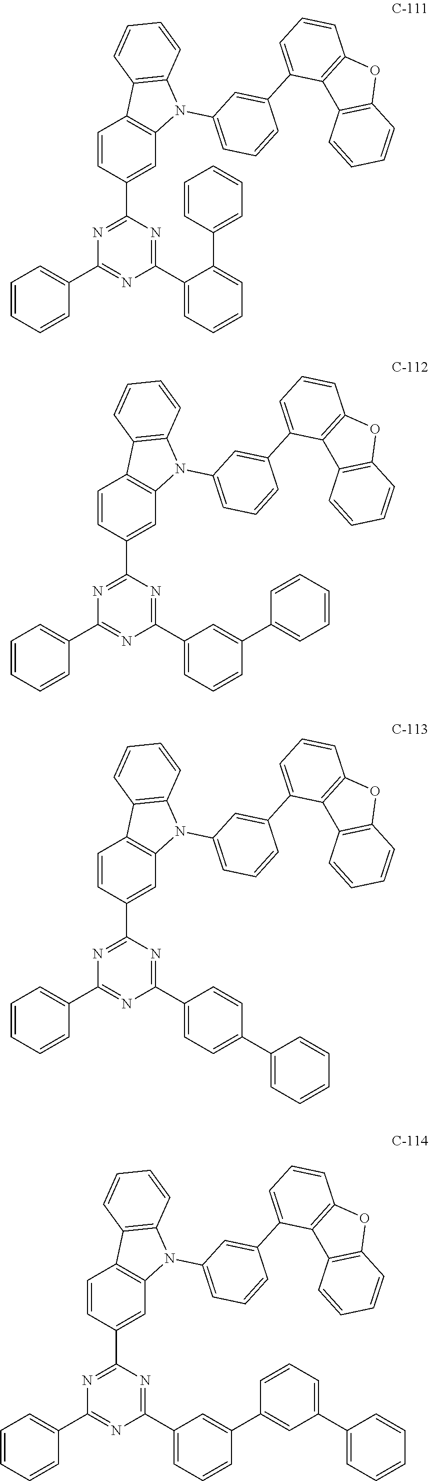 [SMILES: c1ccc(-c2nc(-c3ccc4c5ccccc5n(-c5cccc(-c6cccc7oc8ccccc8c67)c5)c4c3)nc(-c3ccccc3-c3ccccc3)n2)cc1 ; c1ccc(-c2cccc(-c3nc(-c4ccccc4)nc(-c4ccc5c6ccccc6n(-c6cccc(-c7cccc8oc9ccccc9c78)c6)c5c4)n3)c2)cc1 ; c1ccc(-c2ccc(-c3nc(-c4ccccc4)nc(-c4ccc5c6ccccc6n(-c6cccc(-c7cccc8oc9ccccc9c78)c6)c5c4)n3)cc2)cc1 ; c1ccc(-c2cccc(-c3cccc(-c4nc(-c5ccccc5)nc(-c5ccc6c7ccccc7n(-c7cccc(-c8cccc9oc%10ccccc%10c89)c7)c6c5)n4)c3)c2)cc1]

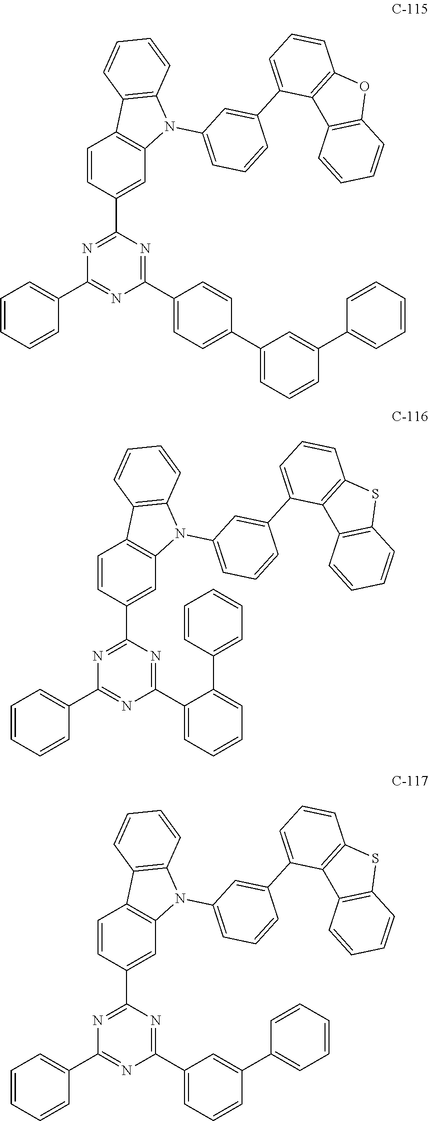

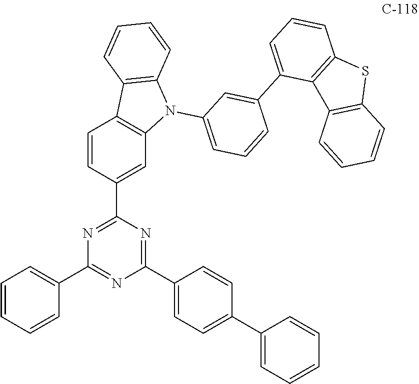 [SMILES: c1ccc(-c2ccc(-c3nc(-c4ccccc4)nc(-c4ccc5c6ccccc6n(-c6cccc(-c7cccc8sc9ccccc9c78)c6)c5c4)n3)cc2)cc1]

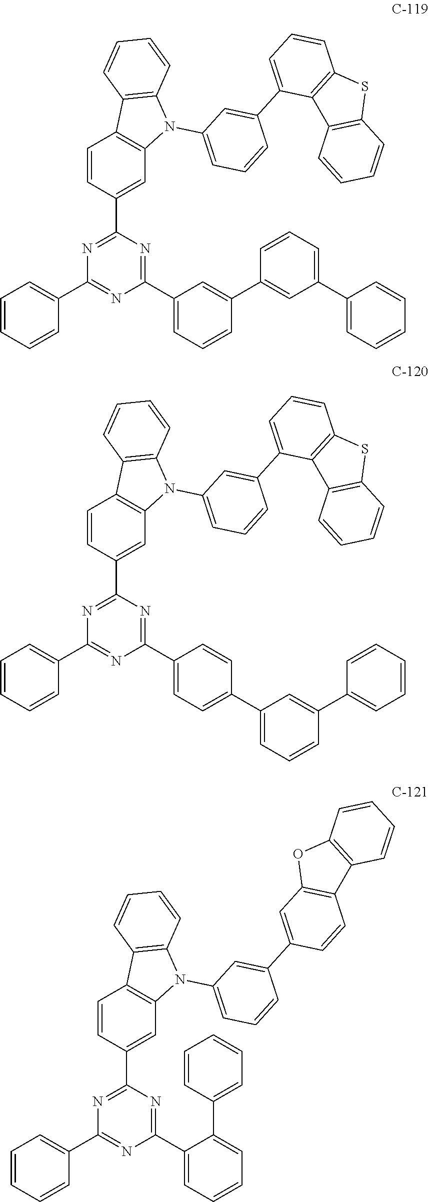 [SMILES: c1ccc(-c2cccc(-c3cccc(-c4nc(-c5ccccc5)nc(-c5ccc6c7ccccc7n(-c7cccc(-c8cccc9sc%10ccccc%10c89)c7)c6c5)n4)c3)c2)cc1 ; c1ccc(-c2cccc(-c3ccc(-c4nc(-c5ccccc5)nc(-c5ccc6c7ccccc7n(-c7cccc(-c8cccc9sc%10ccccc%10c89)c7)c6c5)n4)cc3)c2)cc1 ; c1ccc(-c2nc(-c3ccc4c5ccccc5n(-c5cccc(-c6ccc7c(c6)oc6ccccc67)c5)c4c3)nc(-c3ccccc3-c3ccccc3)n2)cc1]

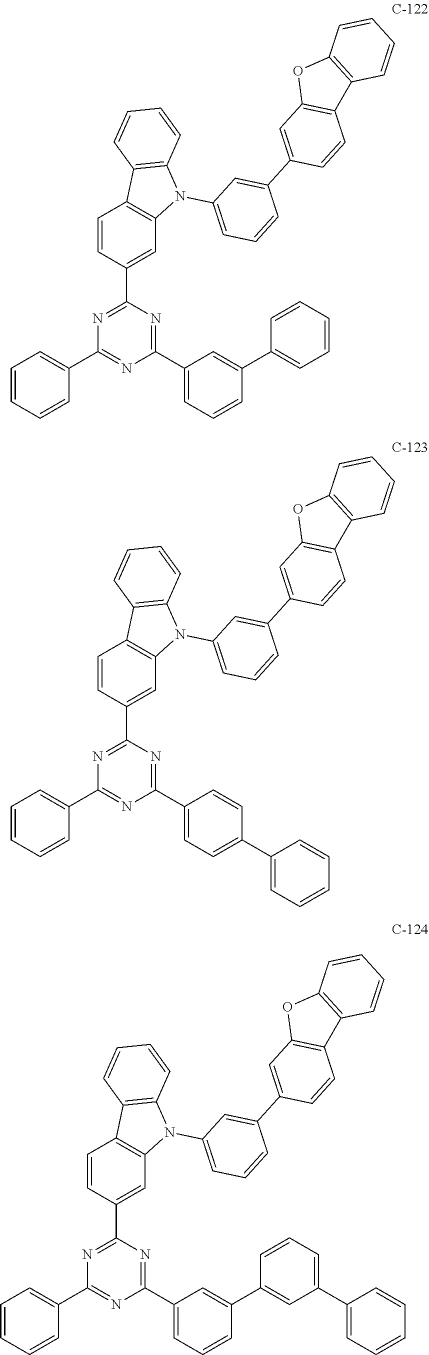 [SMILES: c1ccc(-c2cccc(-c3nc(-c4ccccc4)nc(-c4ccc5c6ccccc6n(-c6cccc(-c7ccc8c(c7)oc7ccccc78)c6)c5c4)n3)c2)cc1 ; c1ccc(-c2ccc(-c3nc(-c4ccccc4)nc(-c4ccc5c6ccccc6n(-c6cccc(-c7ccc8c(c7)oc7ccccc78)c6)c5c4)n3)cc2)cc1 ; c1ccc(-c2cccc(-c3cccc(-c4nc(-c5ccccc5)nc(-c5ccc6c7ccccc7n(-c7cccc(-c8ccc9c(c8)oc8ccccc89)c7)c6c5)n4)c3)c2)cc1]

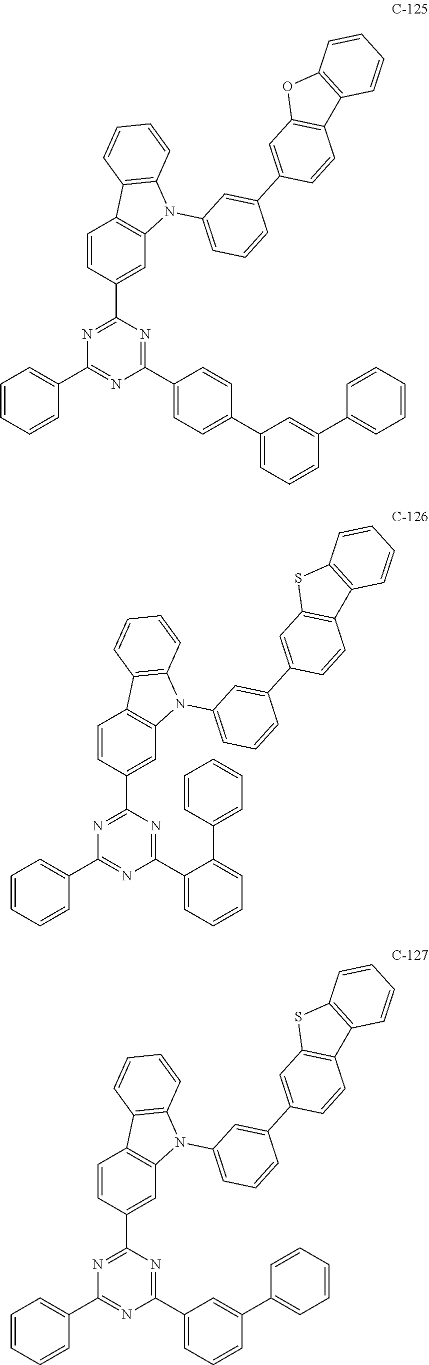 [SMILES: c1ccc(-c2cccc(-c3ccc(-c4nc(-c5ccccc5)nc(-c5ccc6c7ccccc7n(-c7cccc(-c8ccc9c(c8)oc8ccccc89)c7)c6c5)n4)cc3)c2)cc1 ; c1ccc(-c2nc(-c3ccc4c5ccccc5n(-c5cccc(-c6ccc7c(c6)sc6ccccc67)c5)c4c3)nc(-c3ccccc3-c3ccccc3)n2)cc1 ; c1ccc(-c2cccc(-c3nc(-c4ccccc4)nc(-c4ccc5c6ccccc6n(-c6cccc(-c7ccc8c(c7)sc7ccccc78)c6)c5c4)n3)c2)cc1]

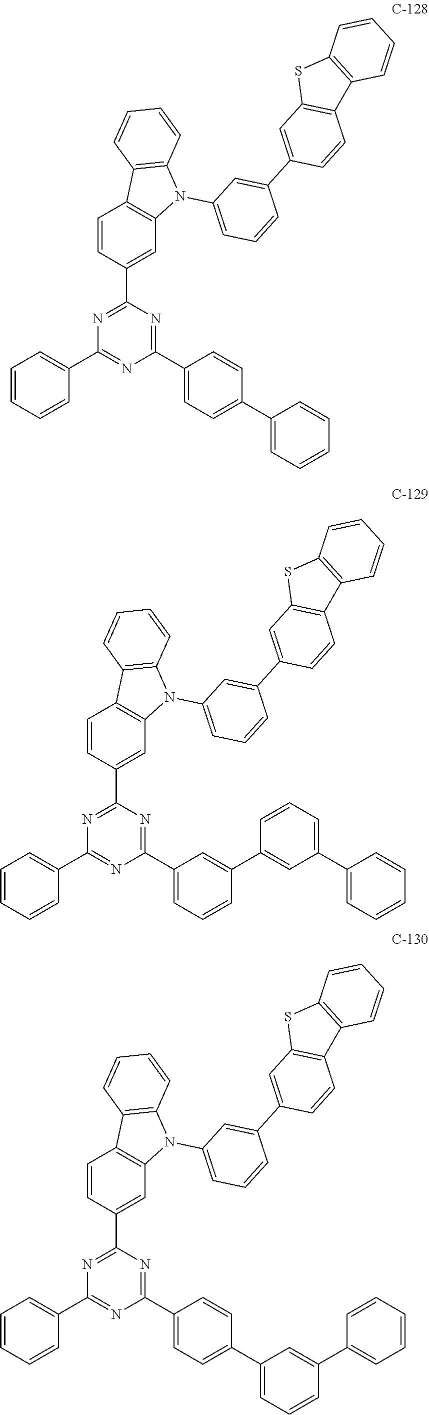 [SMILES: c1ccc(-c2ccc(-c3nc(-c4ccccc4)nc(-c4ccc5c6ccccc6n(-c6cccc(-c7ccc8c(c7)sc7ccccc78)c6)c5c4)n3)cc2)cc1 ; c1ccc(-c2cccc(-c3cccc(-c4nc(-c5ccccc5)nc(-c5ccc6c7ccccc7n(-c7cccc(-c8ccc9c(c8)sc8ccccc89)c7)c6c5)n4)c3)c2)cc1 ; c1ccc(-c2cccc(-c3ccc(-c4nc(-c5ccccc5)nc(-c5ccc6c7ccccc7n(-c7cccc(-c8ccc9c(c8)sc8ccccc89)c7)c6c5)n4)cc3)c2)cc1]

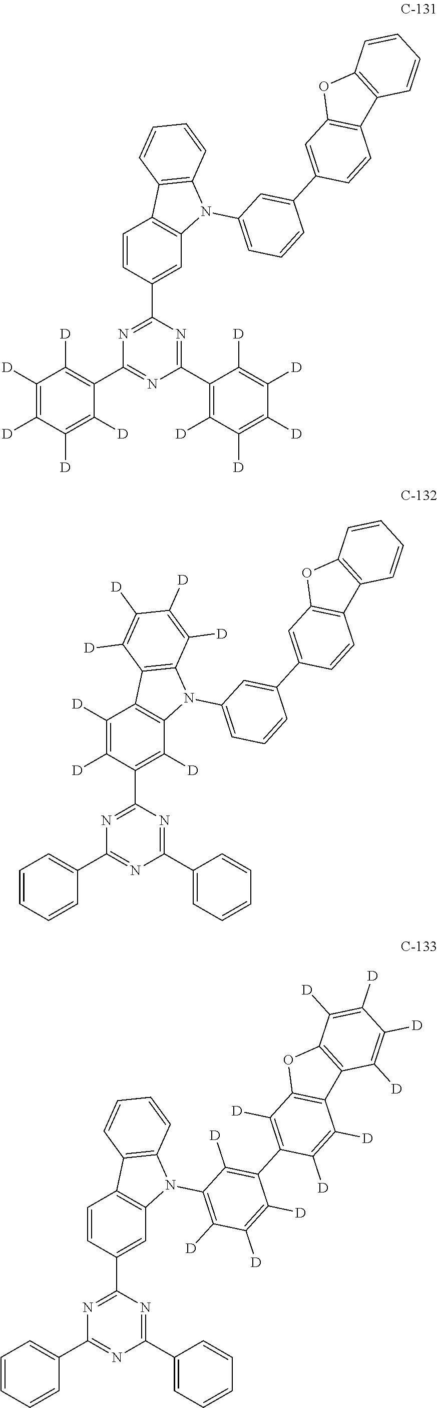 [SMILES: [2H]c1c([2H])c([2H])c(-c2nc(-c3ccc4c5ccccc5n(-c5cccc(-c6ccc7c(c6)oc6ccccc67)c5)c4c3)nc(-c3c([2H])c([2H])c([2H])c([2H])c3[2H])n2)c([2H])c1[2H] ; [2H]c1c([2H])c([2H])c2c(c1[2H])c1c([2H])c([2H])c(-c3nc(-c4ccccc4)nc(-c4ccccc4)n3)c([2H])c1n2-c1cccc(-c2ccc3c(c2)oc2ccccc23)c1 ; [2H]c1c([2H])c(-c2c([2H])c([2H])c3c(oc4c([2H])c([2H])c([2H])c([2H])c43)c2[2H])c([2H])c(-n2c3ccccc3c3ccc(-c4nc(-c5ccccc5)nc(-c5ccccc5)n4)cc32)c1[2H]]

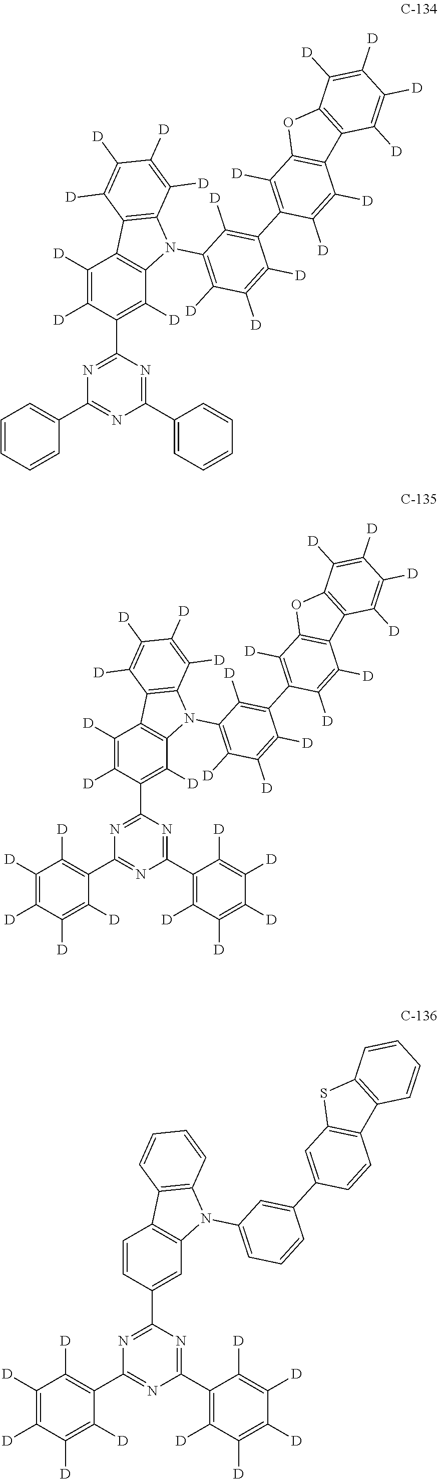

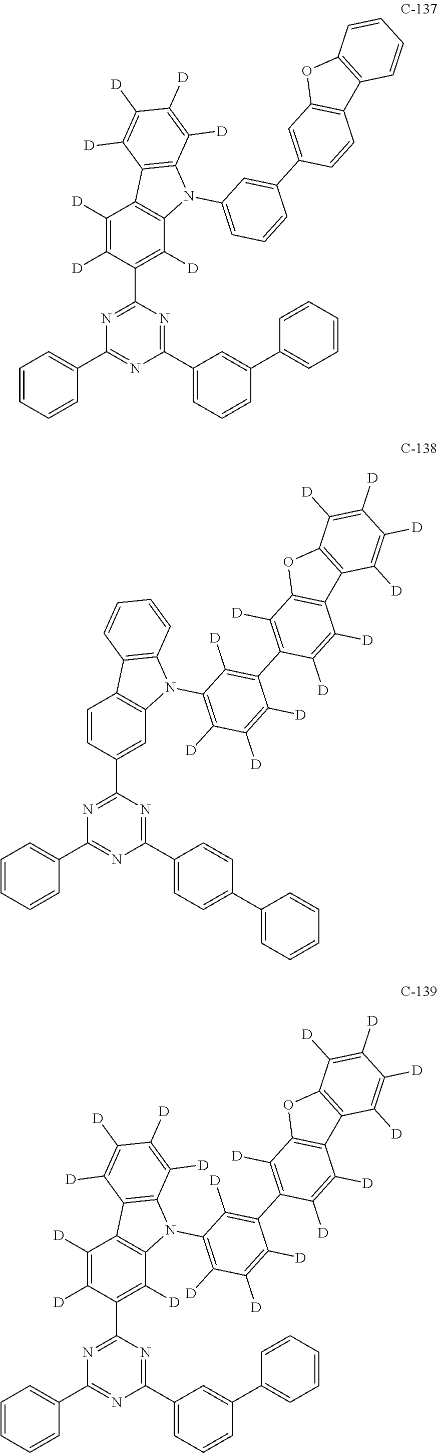 [SMILES: [2H]c1c([2H])c([2H])c2c(c1[2H])c1c([2H])c([2H])c(-c3nc(-c4ccccc4)nc(-c4cccc(-c5ccccc5)c4)n3)c([2H])c1n2-c1cccc(-c2ccc3c(c2)oc2ccccc23)c1 ; [2H]c1c([2H])c(-c2c([2H])c([2H])c3c(oc4c([2H])c([2H])c([2H])c([2H])c43)c2[2H])c([2H])c(-n2c3ccccc3c3ccc(-c4nc(-c5ccccc5)nc(-c5ccc(-c6ccccc6)cc5)n4)cc32)c1[2H] ; [2H]c1c([2H])c(-c2c([2H])c([2H])c3c(oc4c([2H])c([2H])c([2H])c([2H])c43)c2[2H])c([2H])c(-n2c3c([2H])c([2H])c([2H])c([2H])c3c3c([2H])c([2H])c(-c4nc(-c5ccccc5)nc(-c5cccc(-c6ccccc6)c5)n4)c([2H])c32)c1[2H]]

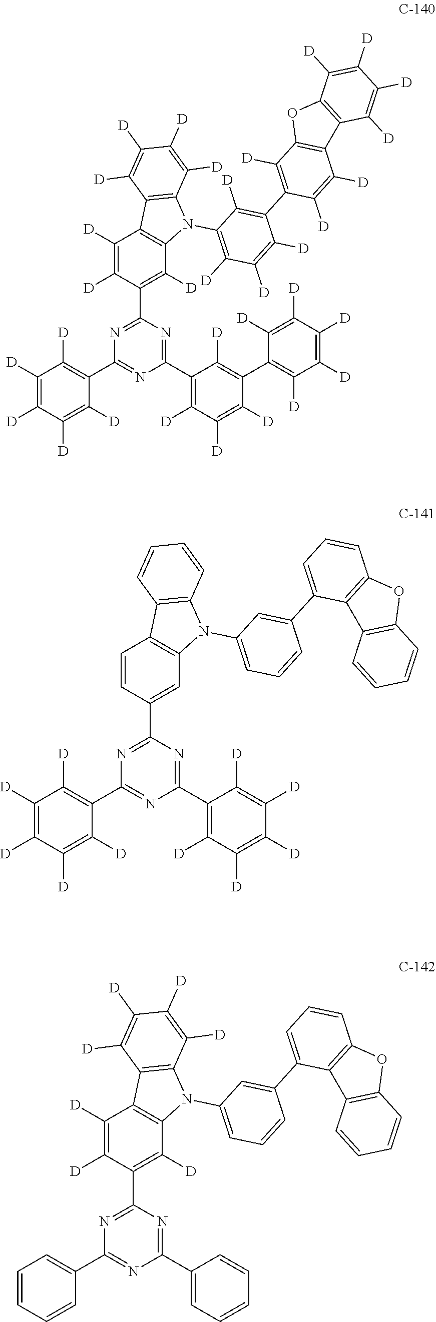

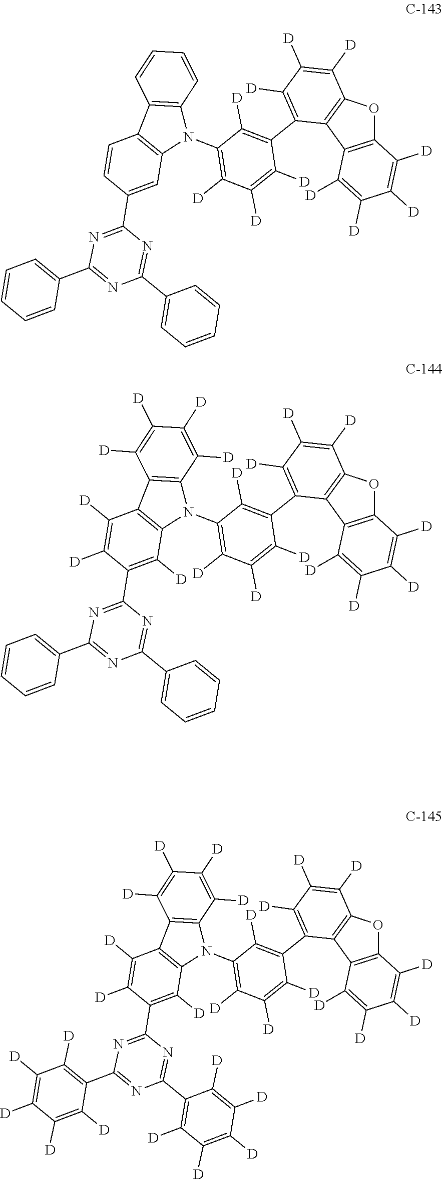

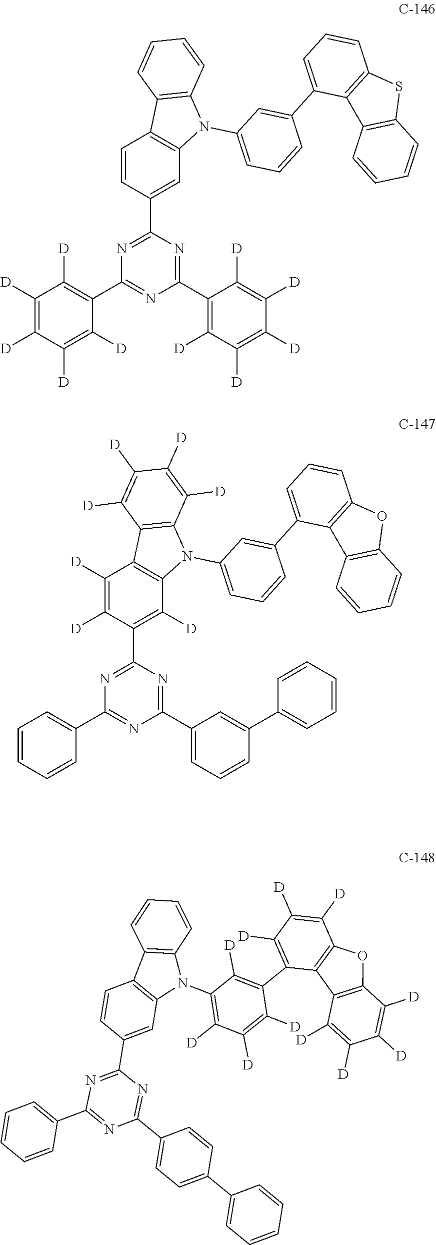 [SMILES: [2H]c1c([2H])c([2H])c(-c2nc(-c3ccc4c5ccccc5n(-c5cccc(-c6cccc7sc8ccccc8c67)c5)c4c3)nc(-c3c([2H])c([2H])c([2H])c([2H])c3[2H])n2)c([2H])c1[2H] ; [2H]c1c([2H])c([2H])c2c(c1[2H])c1c([2H])c([2H])c(-c3nc(-c4ccccc4)nc(-c4cccc(-c5ccccc5)c4)n3)c([2H])c1n2-c1cccc(-c2cccc3oc4ccccc4c23)c1 ; [2H]c1c([2H])c(-c2c([2H])c([2H])c([2H])c3oc4c([2H])c([2H])c([2H])c([2H])c4c23)c([2H])c(-n2c3ccccc3c3ccc(-c4nc(-c5ccccc5)nc(-c5ccc(-c6ccccc6)cc5)n4)cc32)c1[2H]]

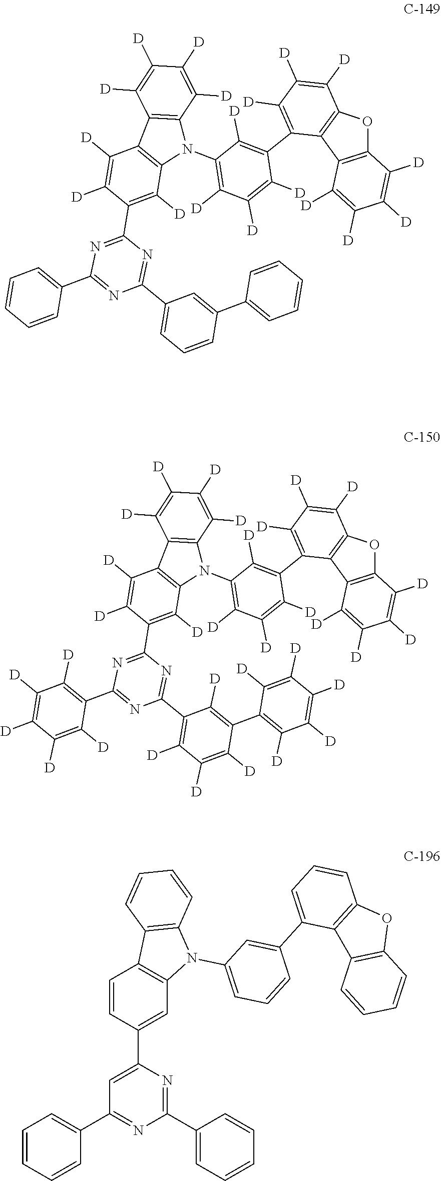

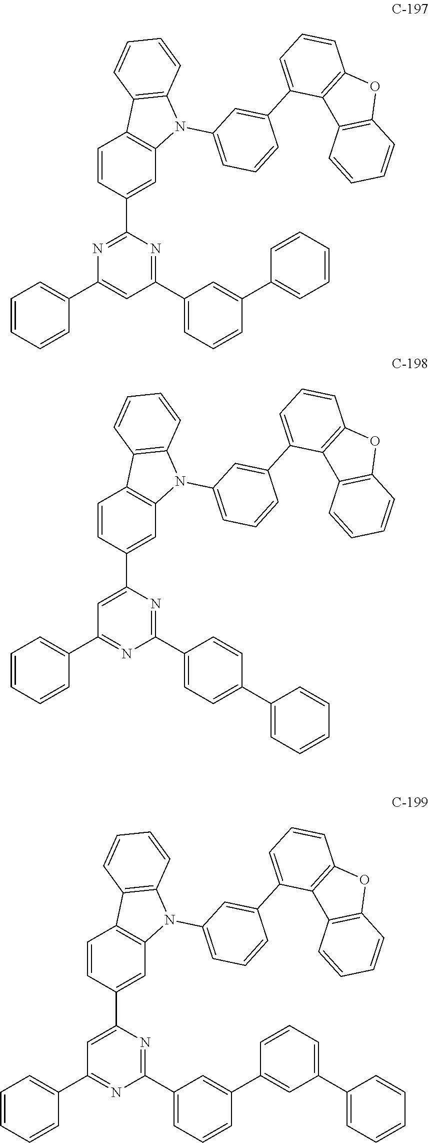 [SMILES: c1ccc(-c2cccc(-c3cc(-c4ccccc4)nc(-c4ccc5c6ccccc6n(-c6cccc(-c7cccc8oc9ccccc9c78)c6)c5c4)n3)c2)cc1 ; c1ccc(-c2ccc(-c3nc(-c4ccccc4)cc(-c4ccc5c6ccccc6n(-c6cccc(-c7cccc8oc9ccccc9c78)c6)c5c4)n3)cc2)cc1 ; c1ccc(-c2cccc(-c3cccc(-c4nc(-c5ccccc5)cc(-c5ccc6c7ccccc7n(-c7cccc(-c8cccc9oc%10ccccc%10c89)c7)c6c5)n4)c3)c2)cc1]

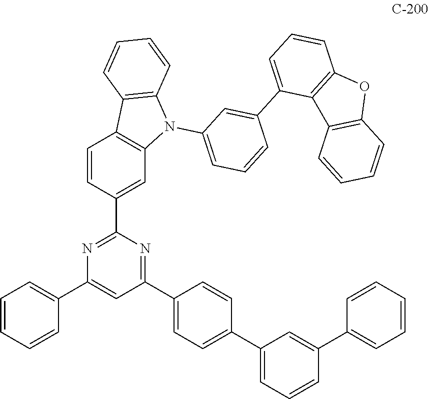 [SMILES: c1ccc(-c2cccc(-c3ccc(-c4cc(-c5ccccc5)nc(-c5ccc6c7ccccc7n(-c7cccc(-c8cccc9oc%10ccccc%10c89)c7)c6c5)n4)cc3)c2)cc1]

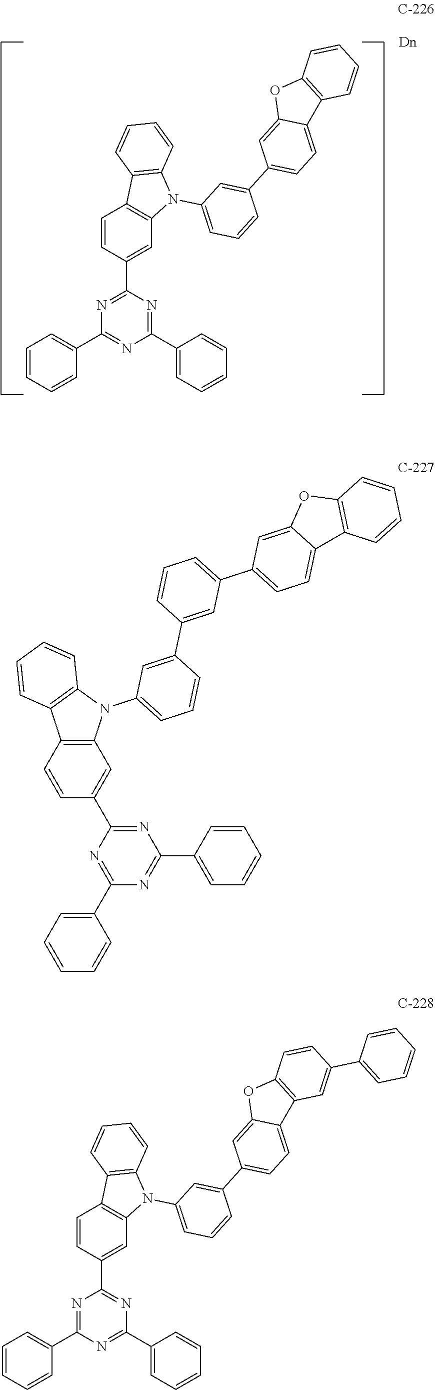

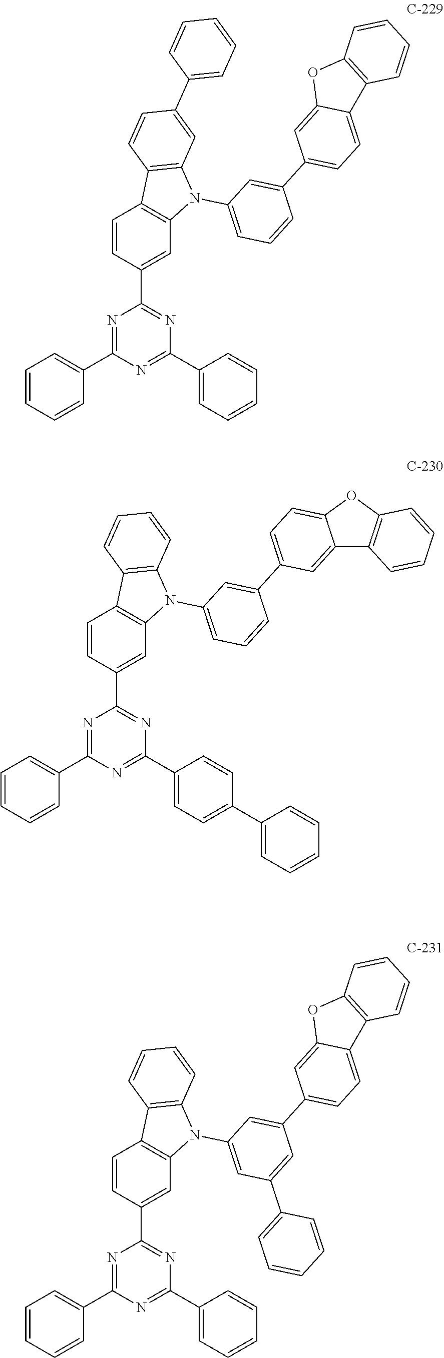 [SMILES: c1ccc(-c2ccc3c4ccc(-c5nc(-c6ccccc6)nc(-c6ccccc6)n5)cc4n(-c4cccc(-c5ccc6c(c5)oc5ccccc56)c4)c3c2)cc1 ; c1ccc(-c2ccc(-c3nc(-c4ccccc4)nc(-c4ccc5c6ccccc6n(-c6cccc(-c7ccc8oc9ccccc9c8c7)c6)c5c4)n3)cc2)cc1 ; c1ccc(-c2cc(-c3ccc4c(c3)oc3ccccc34)cc(-n3c4ccccc4c4ccc(-c5nc(-c6ccccc6)nc(-c6ccccc6)n5)cc43)c2)cc1]

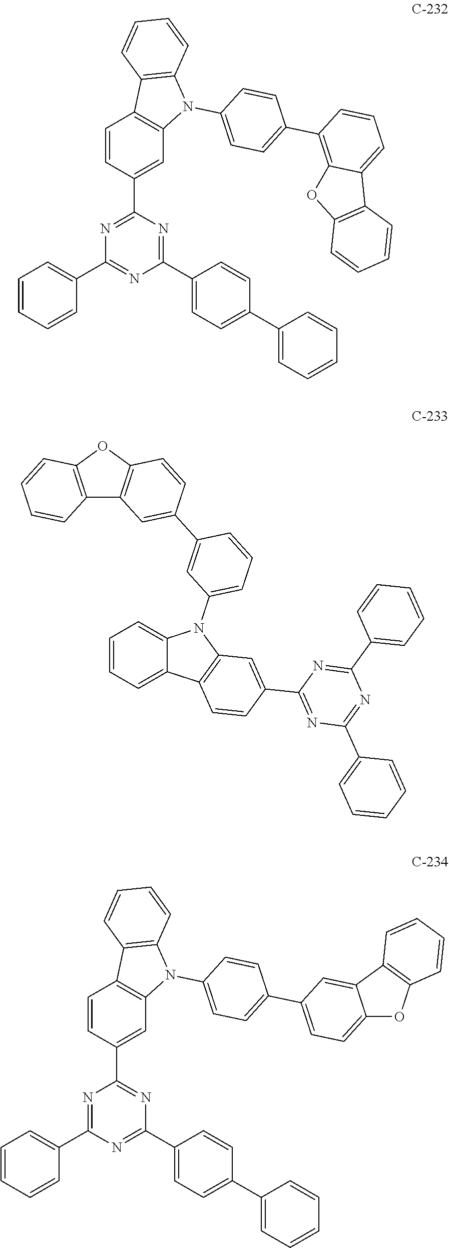 [SMILES: c1ccc(-c2ccc(-c3nc(-c4ccccc4)nc(-c4ccc5c6ccccc6n(-c6ccc(-c7cccc8c7oc7ccccc78)cc6)c5c4)n3)cc2)cc1 ; c1ccc(-c2nc(-c3ccccc3)nc(-c3ccc4c5ccccc5n(-c5cccc(-c6ccc7oc8ccccc8c7c6)c5)c4c3)n2)cc1 ; c1ccc(-c2ccc(-c3nc(-c4ccccc4)nc(-c4ccc5c6ccccc6n(-c6ccc(-c7ccc8oc9ccccc9c8c7)cc6)c5c4)n3)cc2)cc1]

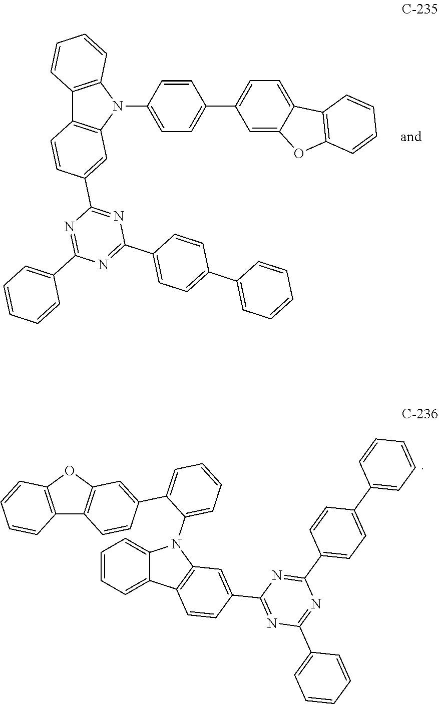 [SMILES: c1ccc(-c2ccc(-c3nc(-c4ccccc4)nc(-c4ccc5c6ccccc6n(-c6ccc(-c7ccc8c(c7)oc7ccccc78)cc6)c5c4)n3)cc2)cc1 ; c1ccc(-c2ccc(-c3nc(-c4ccccc4)nc(-c4ccc5c6ccccc6n(-c6ccccc6-c6ccc7c(c6)oc6ccccc67)c5c4)n3)cc2)cc1]

18. A plurality of host materials comprising at least one first host compound and at least one second host compound, wherein the first host compound is represented by the following Formula 12, the second host compound is represented by the following Formula 13, and the first host compound and the second host compound do not contain deuterium. 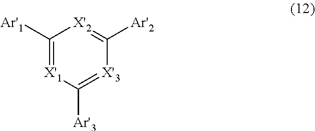
Wherein,
X′1 to X′₃ each independently represent N or CH; provided that at least two of X′₁ to X′₃ represent N;
Ar′₁ to Ar′₃ each independently represent (C6-C30)aryl unsubstituted or substituted with (C6-C30)aryl, or are represented by the following Formula A; provided that at least one of Ar′₁ to Ar′₃ is(are) represented by the following Formula A; 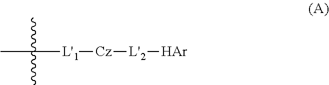
Wherein,
L′₁ represents a single bond, or a substituted or unsubstituted (C6-C12)arylene;
Cz represents a substituted or unsubstituted carbazolyene;
L′₂ represents a substituted or unsubstituted (C6-C12)arylene; and
HAr represents a substituted or unsubstituted dibenzofuranyl, a substituted or unsubstituted carbazolyl, or a substituted or unsubstituted dibenzothiophenyl; 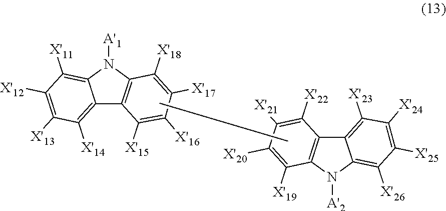
wherein,
A′₁ and A′₂ each independently represent a substituted or unsubstituted (C6-C30)aryl, a substituted or unsubstituted dibenzofuranyl, a substituted or unsubstituted dibenzothiophenyl, or a substituted or unsubstituted carbazoly;
any one of X′₁₅ to X′₁₈ and any one of X′₁₉ to X′₂₂ are connected to each other to form a single bond;
X′₁₁ to X′₁₄, X′₂₃ to X′₂₆, and X′₁₅ to X′₂₂, which do not form a single bond, each independently represent hydrogen, a substituted or unsubstituted (C6-C30)aryl, or a substituted or unsubstituted (3- to 30-membered)heteroaryl; or may be linked to an adjacent substituent to form a ring(s).

19. The plurality of host materials according to claim 18, wherein the compound represented by the Formula 13 is at least one selected from the following compounds.

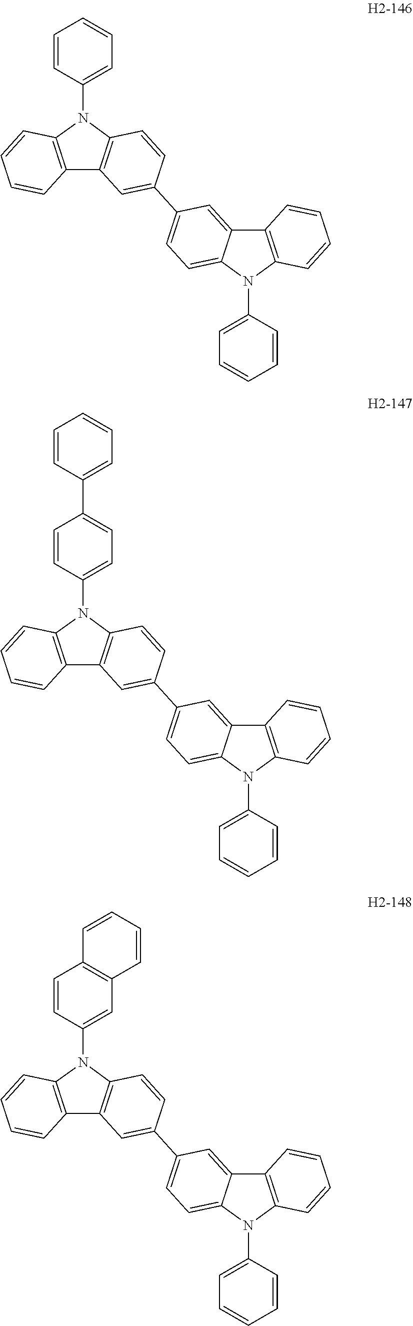 [SMILES: c1ccc(-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccccc5)ccc43)cc2)cc1 ; c1ccc(-n2c3ccccc3c3cc(-c4ccc5c(c4)c4ccccc4n5-c4ccc5ccccc5c4)ccc32)cc1]

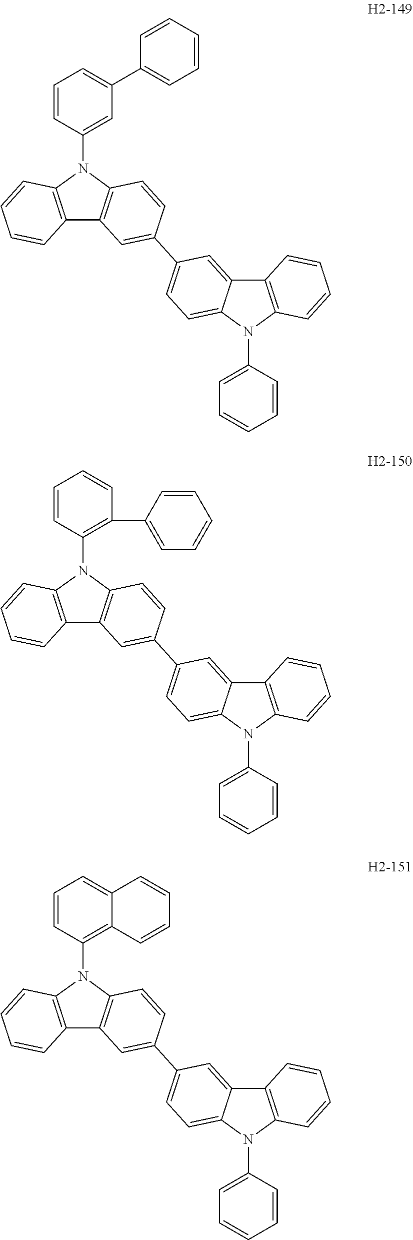 [SMILES: c1ccc(-c2cccc(-n3c4ccccc4c4cc(-c5ccc6c(c5)c5ccccc5n6-c5ccccc5)ccc43)c2)cc1 ; c1ccc(-c2ccccc2-n2c3ccccc3c3cc(-c4ccc5c(c4)c4ccccc4n5-c4ccccc4)ccc32)cc1 ; c1ccc(-n2c3ccccc3c3cc(-c4ccc5c(c4)c4ccccc4n5-c4cccc5ccccc45)ccc32)cc1]

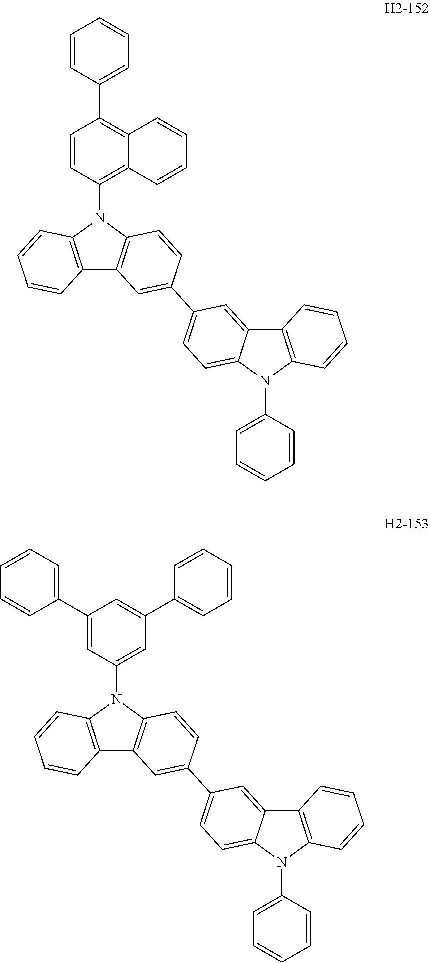 [SMILES: c1ccc(-c2ccc(-n3c4ccccc4c4cc(-c5ccc6c(c5)c5ccccc5n6-c5ccccc5)ccc43)c3ccccc23)cc1 ; c1ccc(-c2cc(-c3ccccc3)cc(-n3c4ccccc4c4cc(-c5ccc6c(c5)c5ccccc5n6-c5ccccc5)ccc43)c2)cc1]

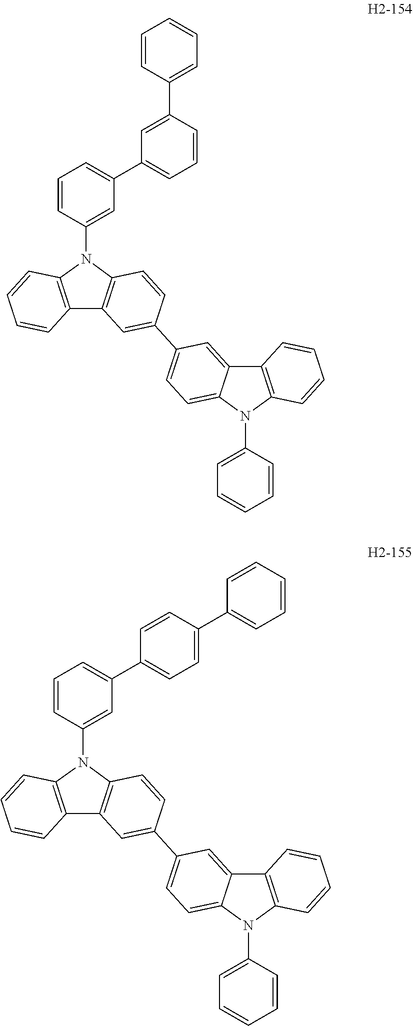 [SMILES: c1ccc(-c2cccc(-c3cccc(-n4c5ccccc5c5cc(-c6ccc7c(c6)c6ccccc6n7-c6ccccc6)ccc54)c3)c2)cc1 ; c1ccc(-c2ccc(-c3cccc(-n4c5ccccc5c5cc(-c6ccc7c(c6)c6ccccc6n7-c6ccccc6)ccc54)c3)cc2)cc1]

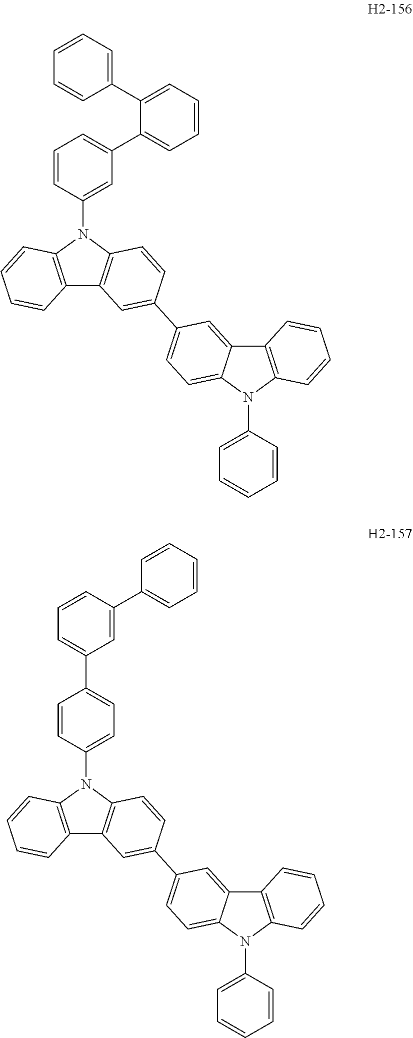 [SMILES: c1ccc(-c2ccccc2-c2cccc(-n3c4ccccc4c4cc(-c5ccc6c(c5)c5ccccc5n6-c5ccccc5)ccc43)c2)cc1 ; c1ccc(-c2cccc(-c3ccc(-n4c5ccccc5c5cc(-c6ccc7c(c6)c6ccccc6n7-c6ccccc6)ccc54)cc3)c2)cc1]

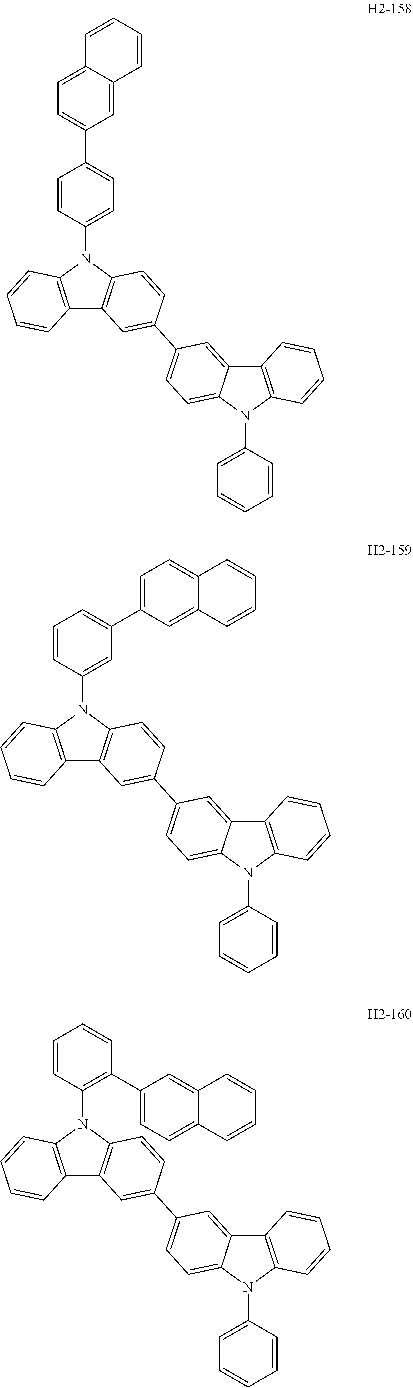 [SMILES: c1ccc(-n2c3ccccc3c3cc(-c4ccc5c(c4)c4ccccc4n5-c4ccc(-c5ccc6ccccc6c5)cc4)ccc32)cc1 ; c1ccc(-n2c3ccccc3c3cc(-c4ccc5c(c4)c4ccccc4n5-c4cccc(-c5ccc6ccccc6c5)c4)ccc32)cc1 ; c1ccc(-n2c3ccccc3c3cc(-c4ccc5c(c4)c4ccccc4n5-c4ccccc4-c4ccc5ccccc5c4)ccc32)cc1]

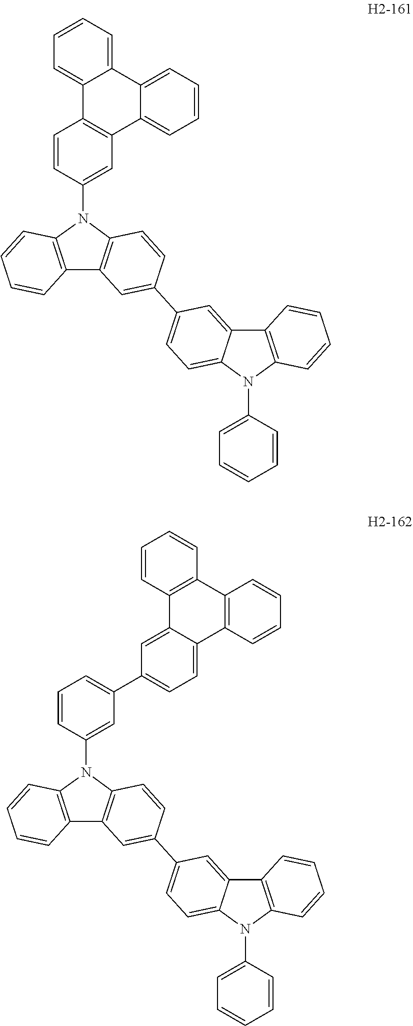 [SMILES: c1ccc(-n2c3ccccc3c3cc(-c4ccc5c(c4)c4ccccc4n5-c4ccc5c6ccccc6c6ccccc6c5c4)ccc32)cc1 ; c1ccc(-n2c3ccccc3c3cc(-c4ccc5c(c4)c4ccccc4n5-c4cccc(-c5ccc6c7ccccc7c7ccccc7c6c5)c4)ccc32)cc1]

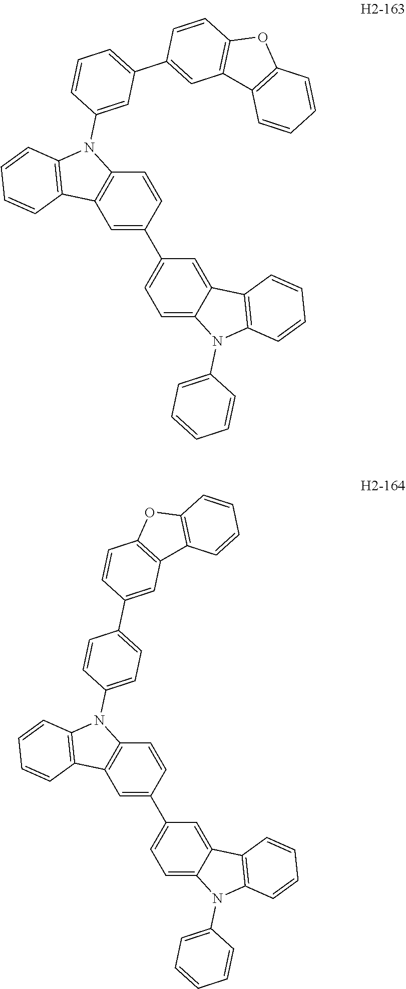 [SMILES: c1ccc(-n2c3ccccc3c3cc(-c4ccc5c(c4)c4ccccc4n5-c4cccc(-c5ccc6oc7ccccc7c6c5)c4)ccc32)cc1 ; c1ccc(-n2c3ccccc3c3cc(-c4ccc5c(c4)c4ccccc4n5-c4ccc(-c5ccc6oc7ccccc7c6c5)cc4)ccc32)cc1]

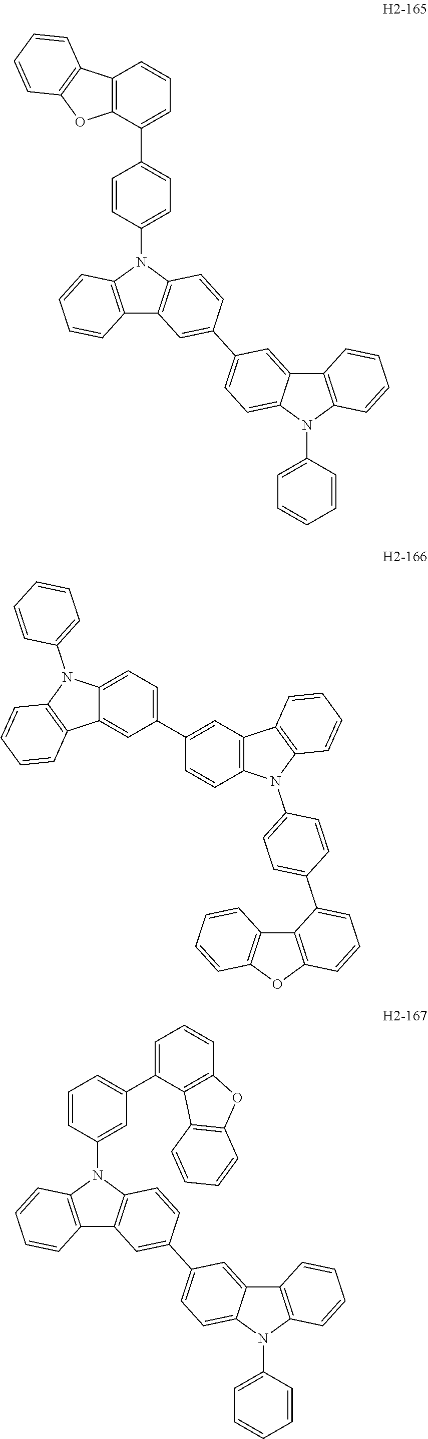 [SMILES: c1ccc(-n2c3ccccc3c3cc(-c4ccc5c(c4)c4ccccc4n5-c4ccc(-c5cccc6c5oc5ccccc56)cc4)ccc32)cc1 ; c1ccc(-n2c3ccccc3c3cc(-c4ccc5c(c4)c4ccccc4n5-c4ccc(-c5cccc6oc7ccccc7c56)cc4)ccc32)cc1 ; c1ccc(-n2c3ccccc3c3cc(-c4ccc5c(c4)c4ccccc4n5-c4cccc(-c5cccc6oc7ccccc7c56)c4)ccc32)cc1]

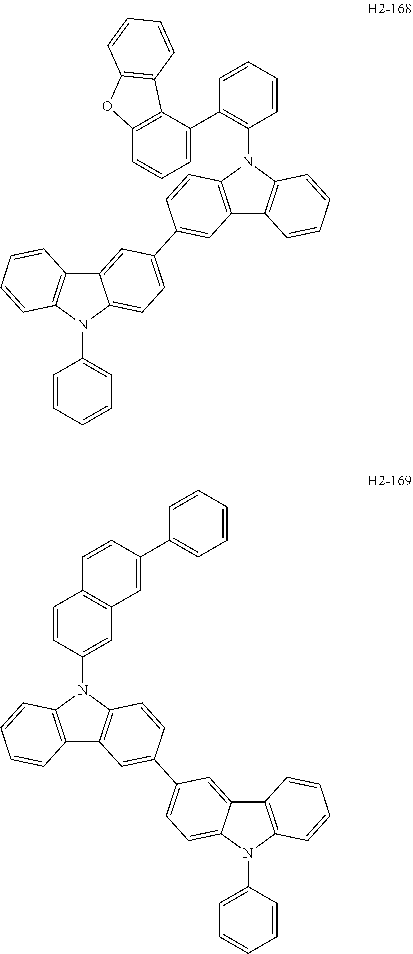 [SMILES: c1ccc(-n2c3ccccc3c3cc(-c4ccc5c(c4)c4ccccc4n5-c4ccccc4-c4cccc5oc6ccccc6c45)ccc32)cc1 ; c1ccc(-c2ccc3ccc(-n4c5ccccc5c5cc(-c6ccc7c(c6)c6ccccc6n7-c6ccccc6)ccc54)cc3c2)cc1]

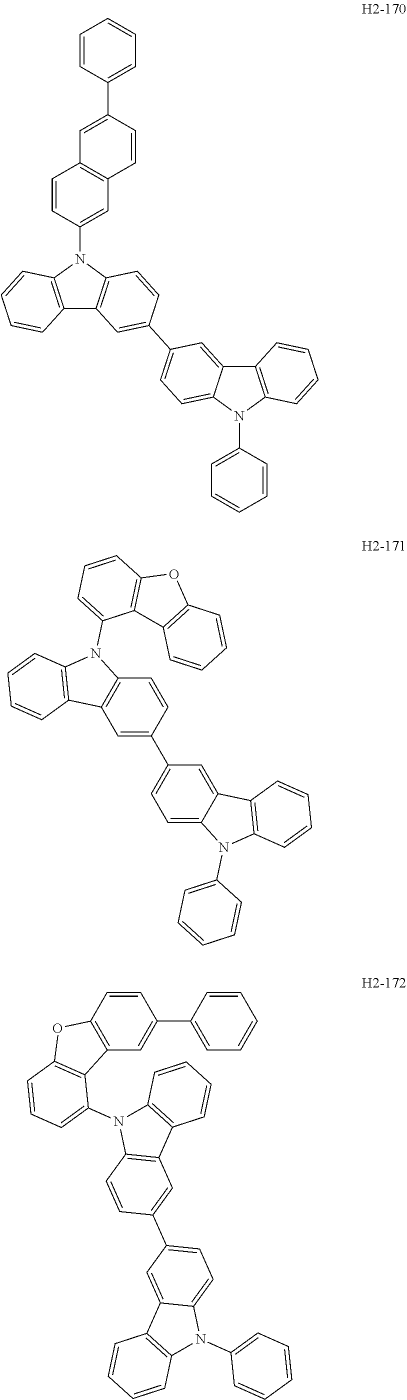 [SMILES: c1ccc(-c2ccc3cc(-n4c5ccccc5c5cc(-c6ccc7c(c6)c6ccccc6n7-c6ccccc6)ccc54)ccc3c2)cc1 ; c1ccc(-n2c3ccccc3c3cc(-c4ccc5c(c4)c4ccccc4n5-c4cccc5oc6ccccc6c45)ccc32)cc1 ; c1ccc(-c2ccc3oc4cccc(-n5c6ccccc6c6cc(-c7ccc8c(c7)c7ccccc7n8-c7ccccc7)ccc65)c4c3c2)cc1]

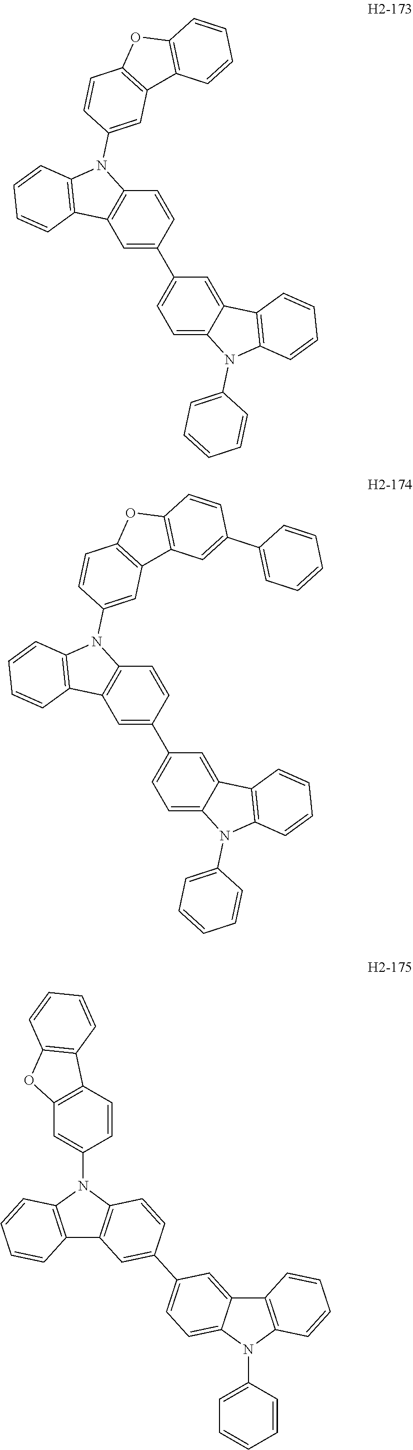

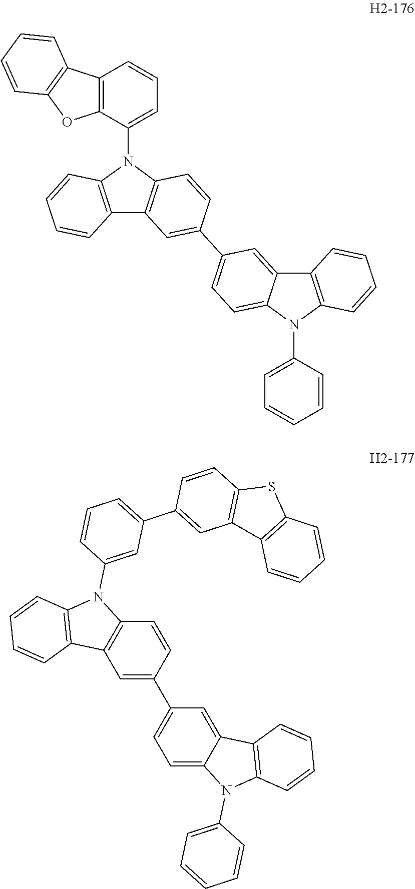 [SMILES: c1ccc(-n2c3ccccc3c3cc(-c4ccc5c(c4)c4ccccc4n5-c4cccc5c4oc4ccccc45)ccc32)cc1 ; c1ccc(-n2c3ccccc3c3cc(-c4ccc5c(c4)c4ccccc4n5-c4cccc(-c5ccc6sc7ccccc7c6c5)c4)ccc32)cc1]

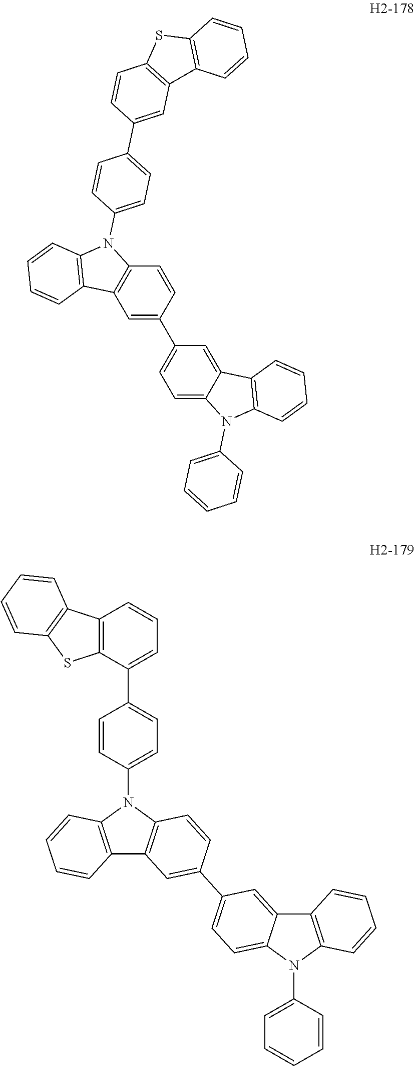 [SMILES: c1ccc(-n2c3ccccc3c3cc(-c4ccc5c(c4)c4ccccc4n5-c4ccc(-c5ccc6sc7ccccc7c6c5)cc4)ccc32)cc1 ; c1ccc(-n2c3ccccc3c3cc(-c4ccc5c(c4)c4ccccc4n5-c4ccc(-c5cccc6c5sc5ccccc56)cc4)ccc32)cc1]

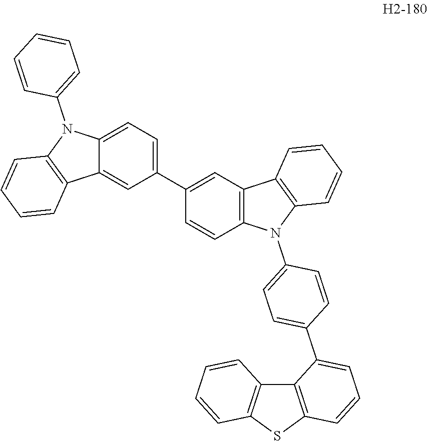 [SMILES: c1ccc(-n2c3ccccc3c3cc(-c4ccc5c(c4)c4ccccc4n5-c4ccc(-c5cccc6sc7ccccc7c56)cc4)ccc32)cc1]

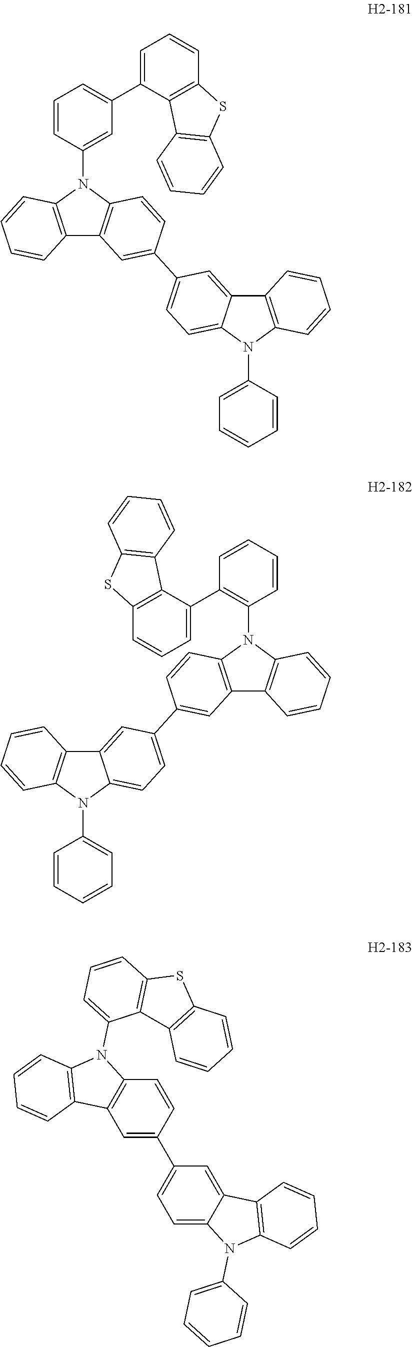 [SMILES: c1ccc(-n2c3ccccc3c3cc(-c4ccc5c(c4)c4ccccc4n5-c4cccc(-c5cccc6sc7ccccc7c56)c4)ccc32)cc1 ; c1ccc(-n2c3ccccc3c3cc(-c4ccc5c(c4)c4ccccc4n5-c4ccccc4-c4cccc5sc6ccccc6c45)ccc32)cc1 ; c1ccc(-n2c3ccccc3c3cc(-c4ccc5c(c4)c4ccccc4n5-c4cccc5sc6ccccc6c45)ccc32)cc1]

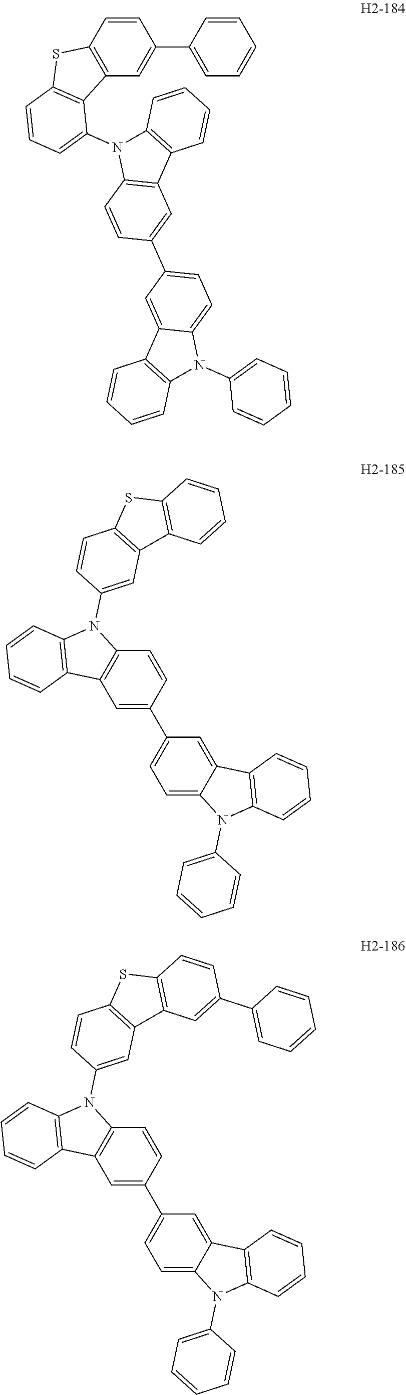

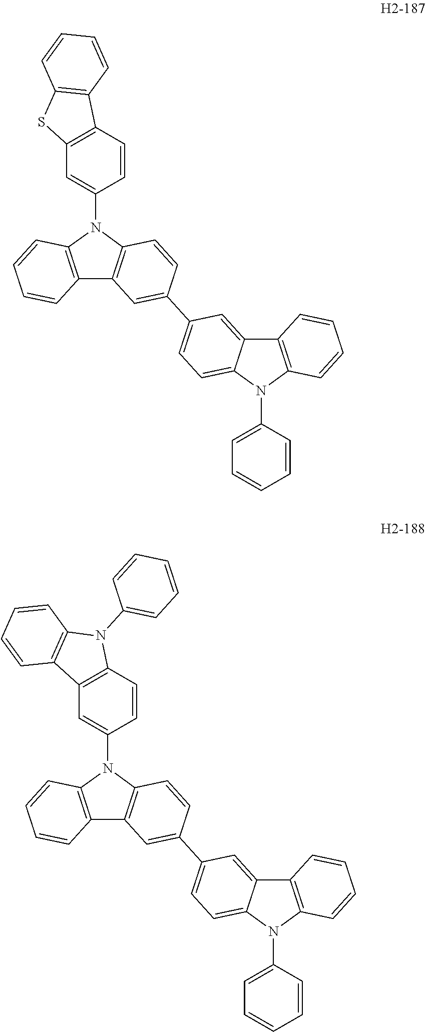 [SMILES: c1ccc(-n2c3ccccc3c3cc(-c4ccc5c(c4)c4ccccc4n5-c4ccc5c(c4)sc4ccccc45)ccc32)cc1 ; c1ccc(-n2c3ccccc3c3cc(-c4ccc5c(c4)c4ccccc4n5-c4ccc5c(c4)c4ccccc4n5-c4ccccc4)ccc32)cc1]

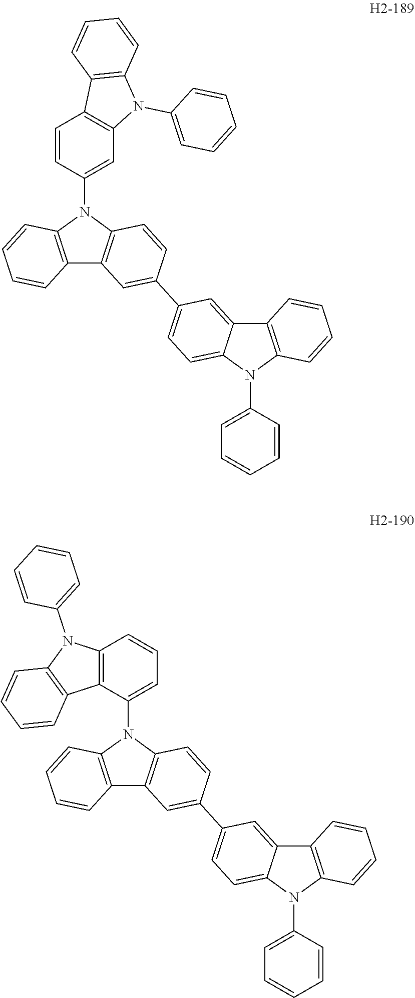 [SMILES: c1ccc(-n2c3ccccc3c3cc(-c4ccc5c(c4)c4ccccc4n5-c4ccc5c6ccccc6n(-c6ccccc6)c5c4)ccc32)cc1 ; c1ccc(-n2c3ccccc3c3cc(-c4ccc5c(c4)c4ccccc4n5-c4cccc5c4c4ccccc4n5-c4ccccc4)ccc32)cc1]

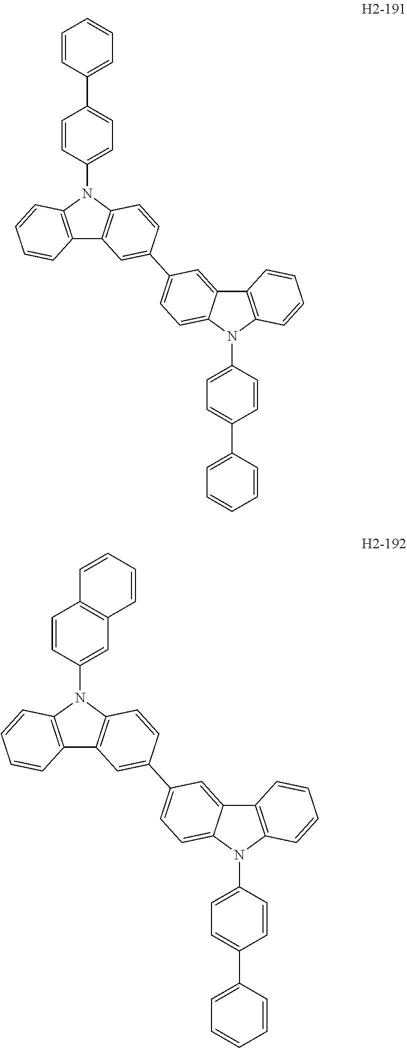 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-n3c4ccccc4c4cc(-c5ccc6c(c5)c5ccccc5n6-c5ccc6ccccc6c5)ccc43)cc2)cc1]

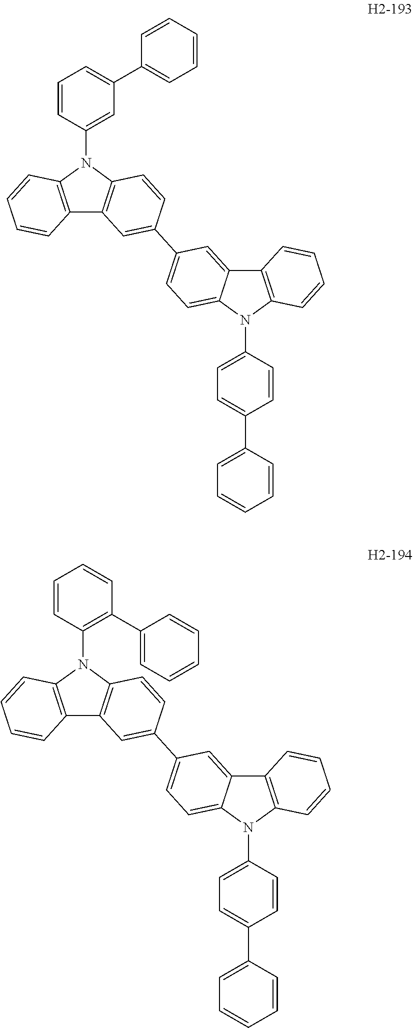 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5-c5ccccc5)ccc43)cc2)cc1]

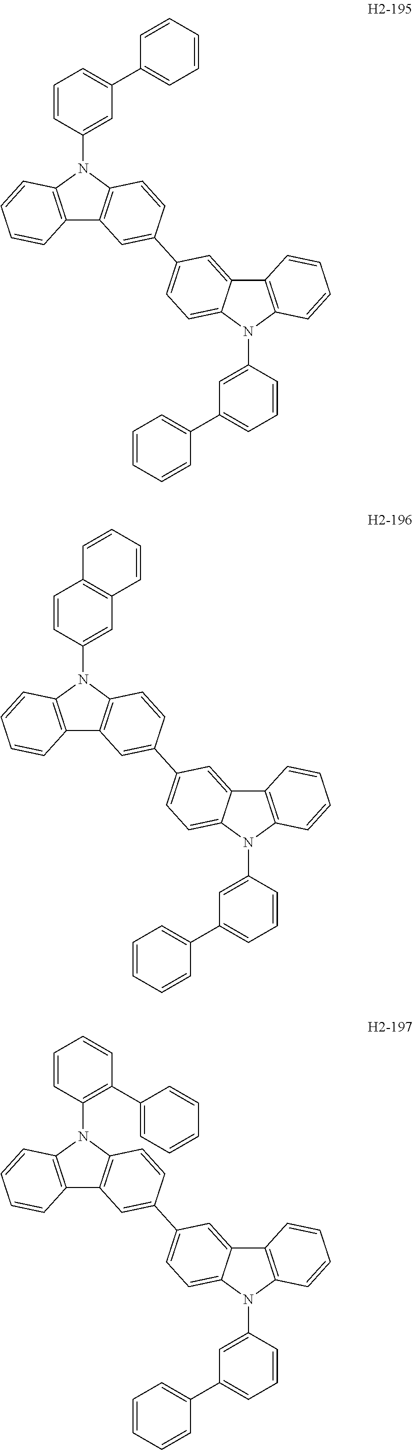 [SMILES: c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2cccc(-n3c4ccccc4c4cc(-c5ccc6c(c5)c5ccccc5n6-c5ccc6ccccc6c5)ccc43)c2)cc1 ; c1ccc(-c2cccc(-n3c4ccccc4c4cc(-c5ccc6c(c5)c5ccccc5n6-c5ccccc5-c5ccccc5)ccc43)c2)cc1]

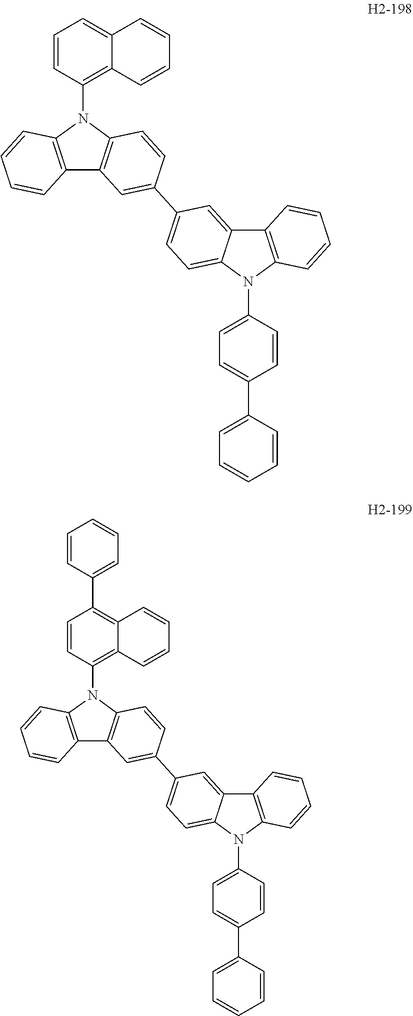 [SMILES: c1ccc(-c2ccc(-n3c4ccccc4c4cc(-c5ccc6c(c5)c5ccccc5n6-c5cccc6ccccc56)ccc43)cc2)cc1 ; c1ccc(-c2ccc(-n3c4ccccc4c4cc(-c5ccc6c(c5)c5ccccc5n6-c5ccc(-c6ccccc6)c6ccccc56)ccc43)cc2)cc1]

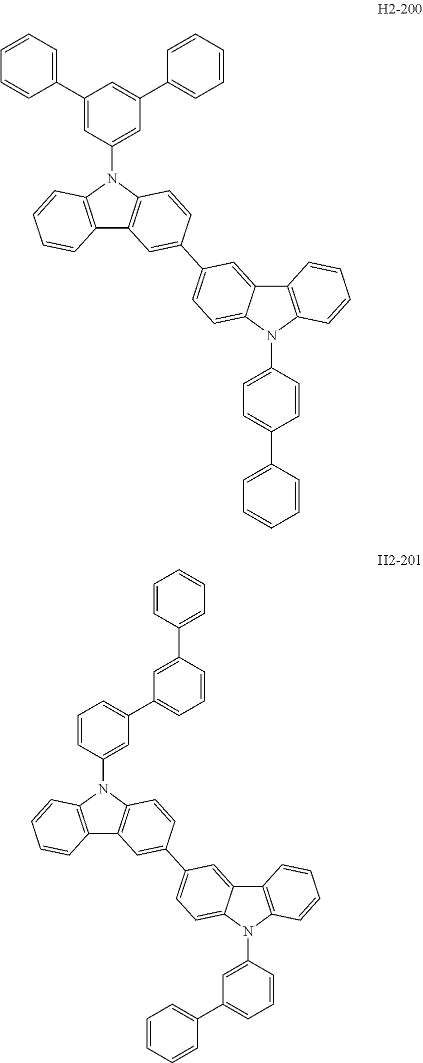 [SMILES: c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1 ; c1ccc(-c2cccc(-c3cccc(-n4c5ccccc5c5cc(-c6ccc7c(c6)c6ccccc6n7-c6cccc(-c7ccccc7)c6)ccc54)c3)c2)cc1]

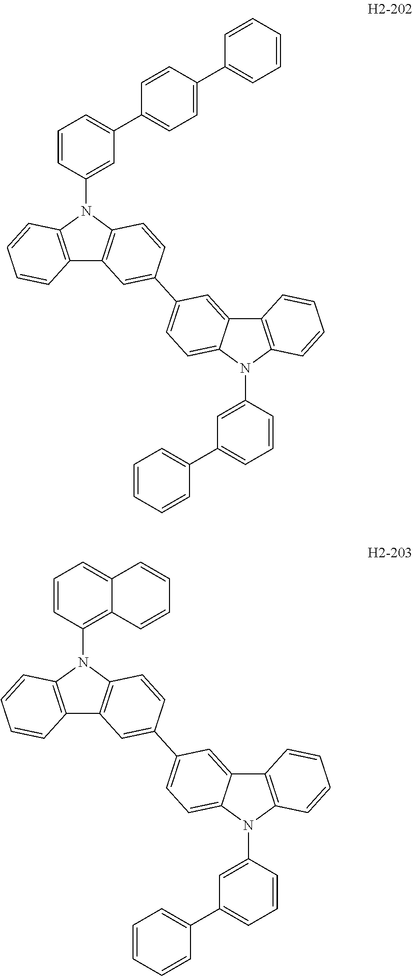 [SMILES: c1ccc(-c2ccc(-c3cccc(-n4c5ccccc5c5cc(-c6ccc7c(c6)c6ccccc6n7-c6cccc(-c7ccccc7)c6)ccc54)c3)cc2)cc1 ; c1ccc(-c2cccc(-n3c4ccccc4c4cc(-c5ccc6c(c5)c5ccccc5n6-c5cccc6ccccc56)ccc43)c2)cc1]

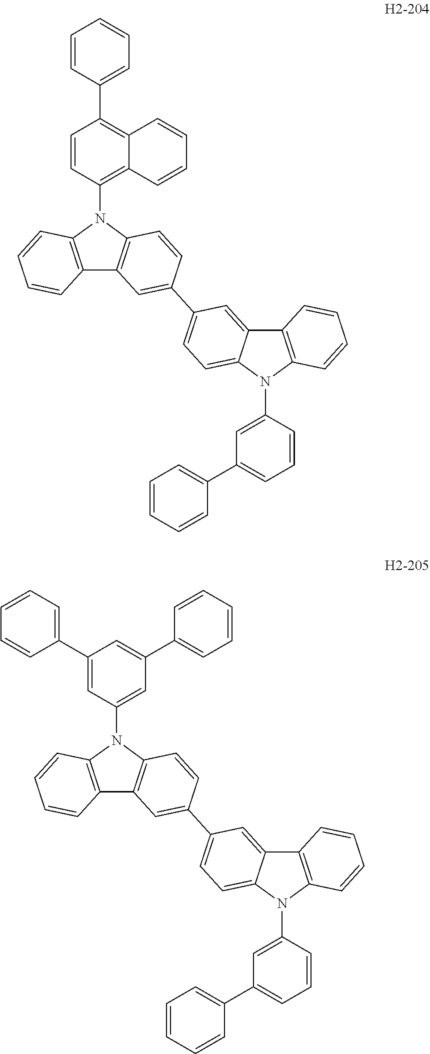 [SMILES: c1ccc(-c2cccc(-n3c4ccccc4c4cc(-c5ccc6c(c5)c5ccccc5n6-c5ccc(-c6ccccc6)c6ccccc56)ccc43)c2)cc1 ; c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1]

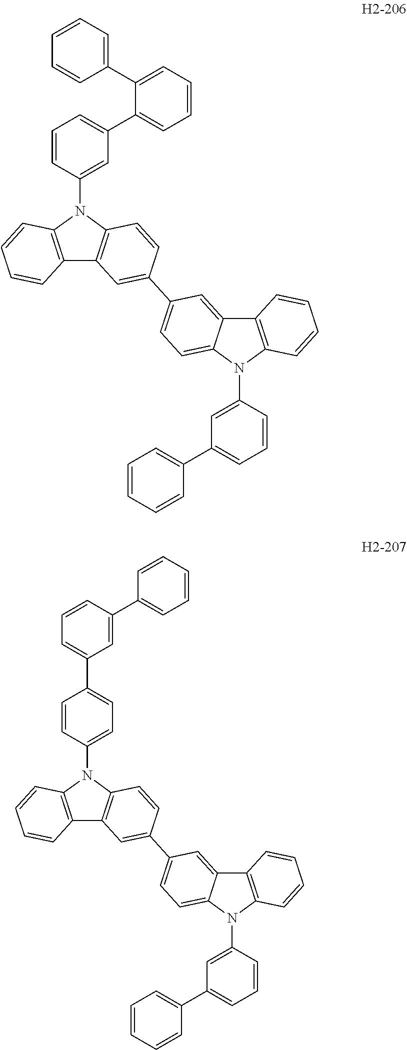 [SMILES: c1ccc(-c2cccc(-n3c4ccccc4c4cc(-c5ccc6c(c5)c5ccccc5n6-c5cccc(-c6ccccc6-c6ccccc6)c5)ccc43)c2)cc1 ; c1ccc(-c2cccc(-c3ccc(-n4c5ccccc5c5cc(-c6ccc7c(c6)c6ccccc6n7-c6cccc(-c7ccccc7)c6)ccc54)cc3)c2)cc1]

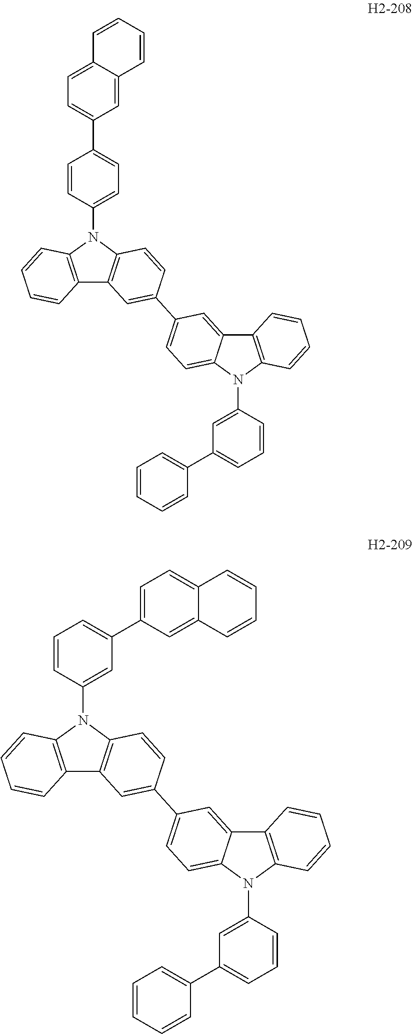 [SMILES: c1ccc(-c2cccc(-n3c4ccccc4c4cc(-c5ccc6c(c5)c5ccccc5n6-c5ccc(-c6ccc7ccccc7c6)cc5)ccc43)c2)cc1 ; c1ccc(-c2cccc(-n3c4ccccc4c4cc(-c5ccc6c(c5)c5ccccc5n6-c5cccc(-c6ccc7ccccc7c6)c5)ccc43)c2)cc1]

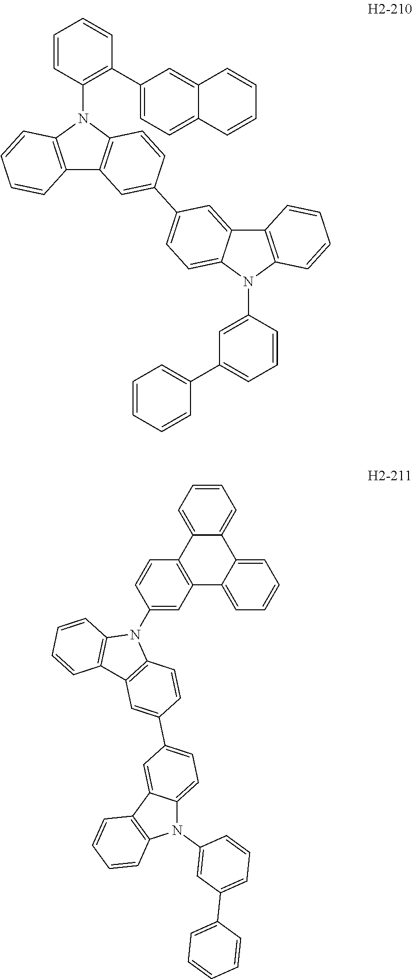 [SMILES: c1ccc(-c2cccc(-n3c4ccccc4c4cc(-c5ccc6c(c5)c5ccccc5n6-c5ccccc5-c5ccc6ccccc6c5)ccc43)c2)cc1 ; c1ccc(-c2cccc(-n3c4ccccc4c4cc(-c5ccc6c(c5)c5ccccc5n6-c5ccc6c7ccccc7c7ccccc7c6c5)ccc43)c2)cc1]

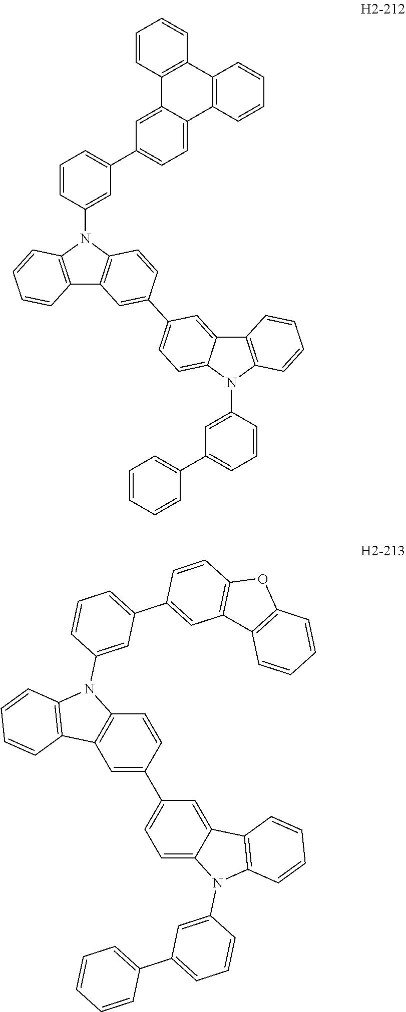 [SMILES: c1ccc(-c2cccc(-n3c4ccccc4c4cc(-c5ccc6c(c5)c5ccccc5n6-c5cccc(-c6ccc7c8ccccc8c8ccccc8c7c6)c5)ccc43)c2)cc1 ; c1ccc(-c2cccc(-n3c4ccccc4c4cc(-c5ccc6c(c5)c5ccccc5n6-c5cccc(-c6ccc7oc8ccccc8c7c6)c5)ccc43)c2)cc1]

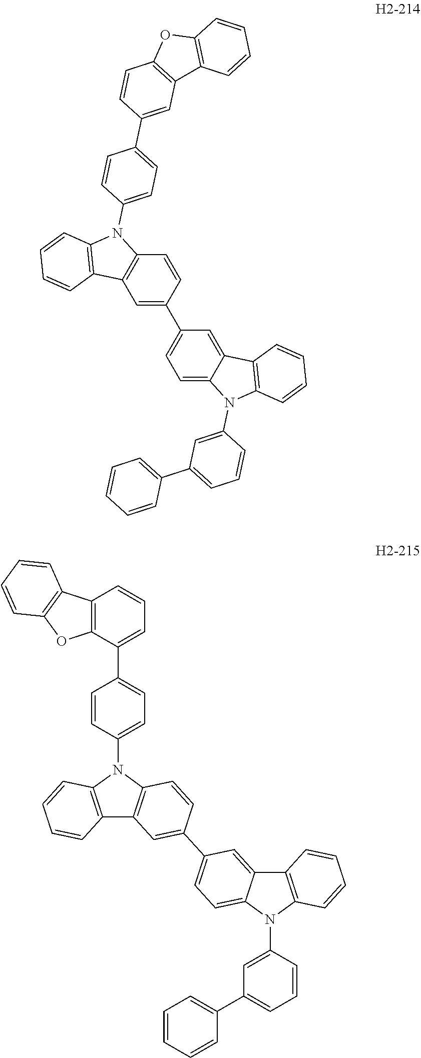 [SMILES: c1ccc(-c2cccc(-n3c4ccccc4c4cc(-c5ccc6c(c5)c5ccccc5n6-c5ccc(-c6ccc7oc8ccccc8c7c6)cc5)ccc43)c2)cc1 ; c1ccc(-c2cccc(-n3c4ccccc4c4cc(-c5ccc6c(c5)c5ccccc5n6-c5ccc(-c6cccc7c6oc6ccccc67)cc5)ccc43)c2)cc1]

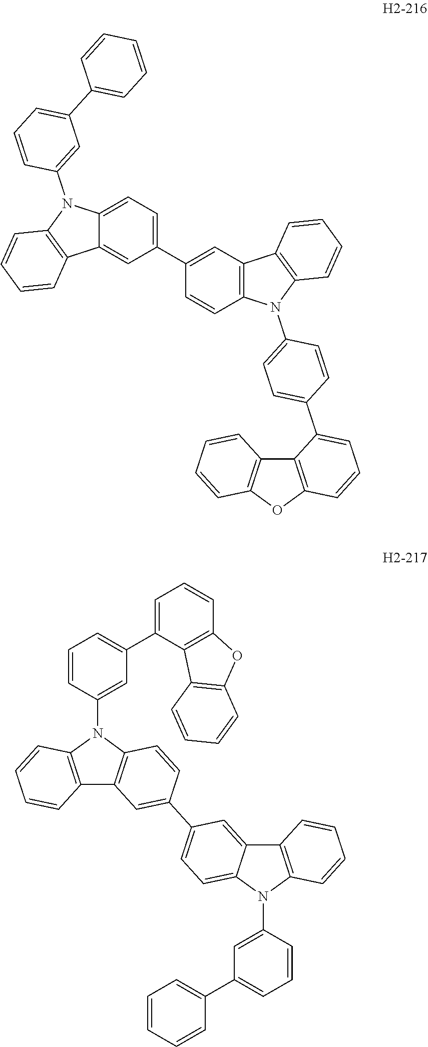 [SMILES: c1ccc(-c2cccc(-n3c4ccccc4c4cc(-c5ccc6c(c5)c5ccccc5n6-c5ccc(-c6cccc7oc8ccccc8c67)cc5)ccc43)c2)cc1 ; c1ccc(-c2cccc(-n3c4ccccc4c4cc(-c5ccc6c(c5)c5ccccc5n6-c5cccc(-c6cccc7oc8ccccc8c67)c5)ccc43)c2)cc1]

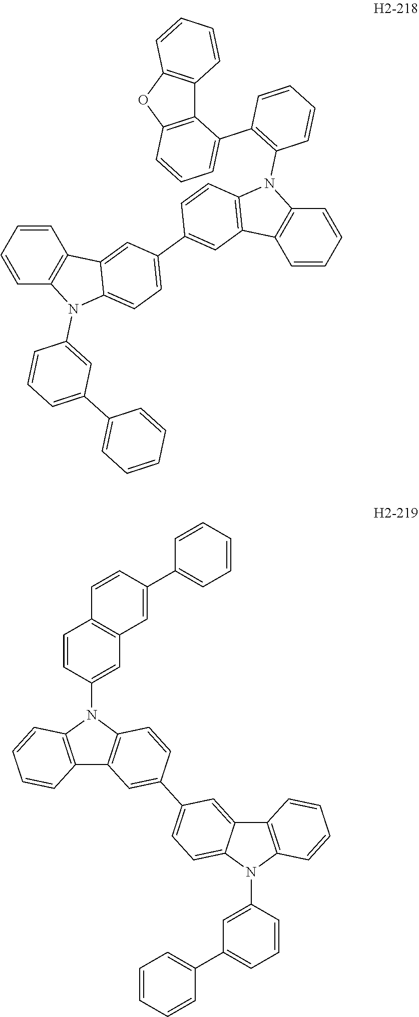 [SMILES: c1ccc(-c2cccc(-n3c4ccccc4c4cc(-c5ccc6c(c5)c5ccccc5n6-c5ccccc5-c5cccc6oc7ccccc7c56)ccc43)c2)cc1 ; c1ccc(-c2cccc(-n3c4ccccc4c4cc(-c5ccc6c(c5)c5ccccc5n6-c5ccc6ccc(-c7ccccc7)cc6c5)ccc43)c2)cc1]

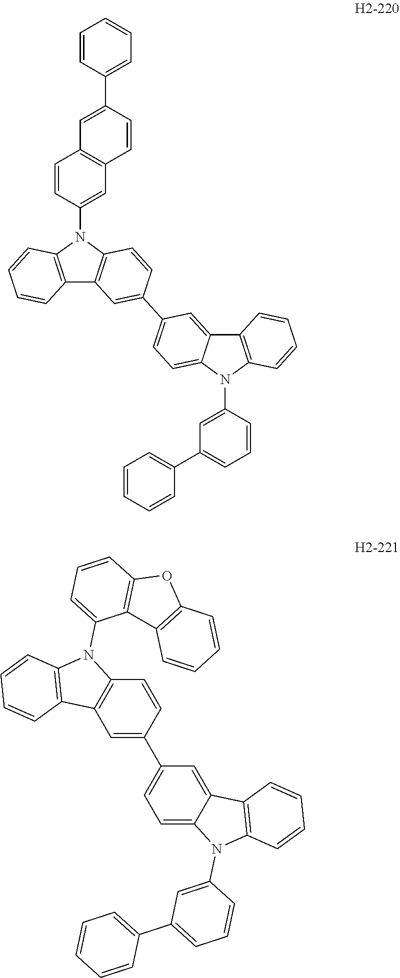 [SMILES: c1ccc(-c2cccc(-n3c4ccccc4c4cc(-c5ccc6c(c5)c5ccccc5n6-c5ccc6cc(-c7ccccc7)ccc6c5)ccc43)c2)cc1 ; c1ccc(-c2cccc(-n3c4ccccc4c4cc(-c5ccc6c(c5)c5ccccc5n6-c5cccc6oc7ccccc7c56)ccc43)c2)cc1]

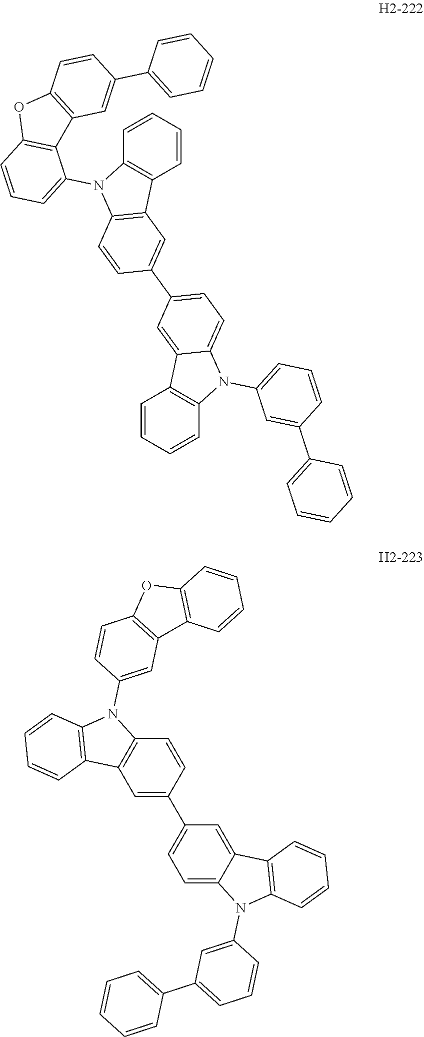 [SMILES: c1ccc(-c2cccc(-n3c4ccccc4c4cc(-c5ccc6c(c5)c5ccccc5n6-c5cccc6oc7ccc(-c8ccccc8)cc7c56)ccc43)c2)cc1 ; c1ccc(-c2cccc(-n3c4ccccc4c4cc(-c5ccc6c(c5)c5ccccc5n6-c5ccc6oc7ccccc7c6c5)ccc43)c2)cc1]

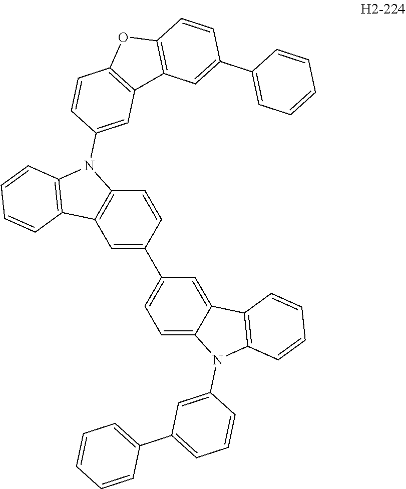 [SMILES: c1ccc(-c2cccc(-n3c4ccccc4c4cc(-c5ccc6c(c5)c5ccccc5n6-c5ccc6oc7ccc(-c8ccccc8)cc7c6c5)ccc43)c2)cc1]

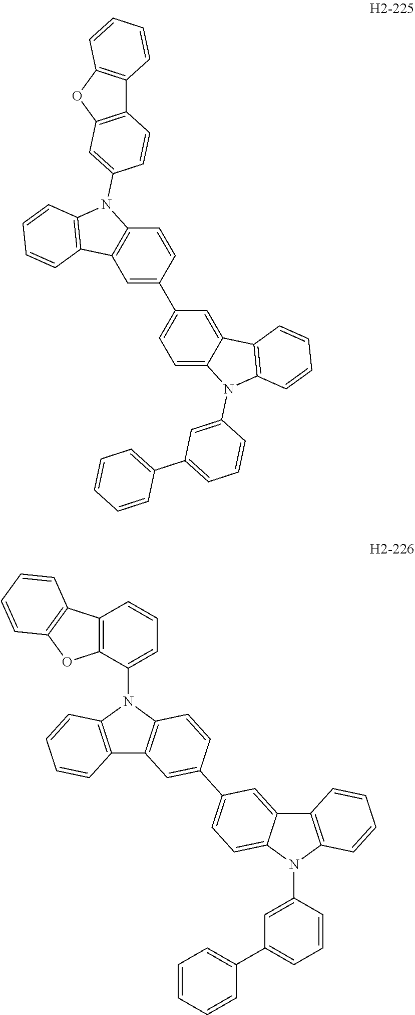 [SMILES: c1ccc(-c2cccc(-n3c4ccccc4c4cc(-c5ccc6c(c5)c5ccccc5n6-c5ccc6c(c5)oc5ccccc56)ccc43)c2)cc1 ; c1ccc(-c2cccc(-n3c4ccccc4c4cc(-c5ccc6c(c5)c5ccccc5n6-c5cccc6c5oc5ccccc56)ccc43)c2)cc1]

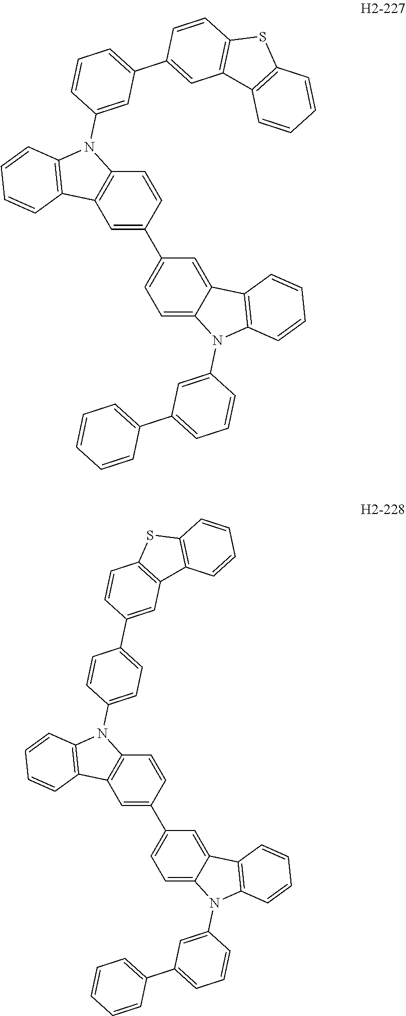 [SMILES: c1ccc(-c2cccc(-n3c4ccccc4c4cc(-c5ccc6c(c5)c5ccccc5n6-c5cccc(-c6ccc7sc8ccccc8c7c6)c5)ccc43)c2)cc1 ; c1ccc(-c2cccc(-n3c4ccccc4c4cc(-c5ccc6c(c5)c5ccccc5n6-c5ccc(-c6ccc7sc8ccccc8c7c6)cc5)ccc43)c2)cc1]

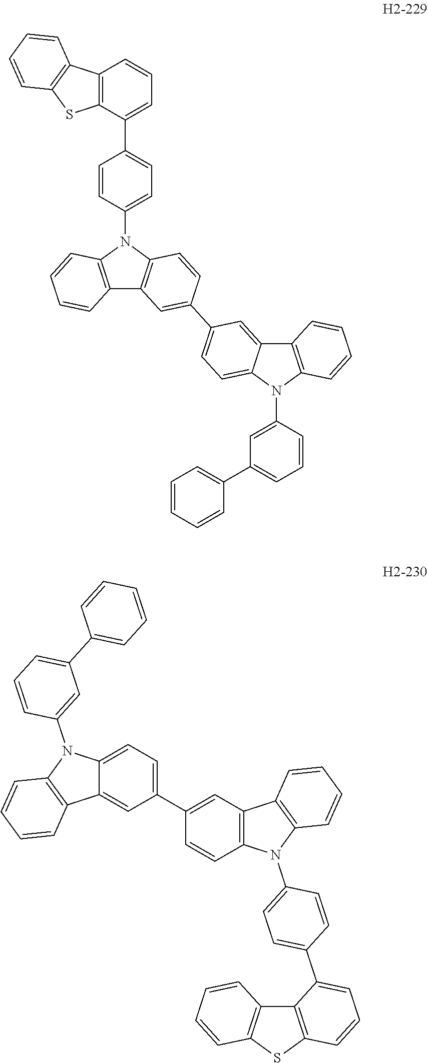 [SMILES: c1ccc(-c2cccc(-n3c4ccccc4c4cc(-c5ccc6c(c5)c5ccccc5n6-c5ccc(-c6cccc7c6sc6ccccc67)cc5)ccc43)c2)cc1 ; c1ccc(-c2cccc(-n3c4ccccc4c4cc(-c5ccc6c(c5)c5ccccc5n6-c5ccc(-c6cccc7sc8ccccc8c67)cc5)ccc43)c2)cc1]

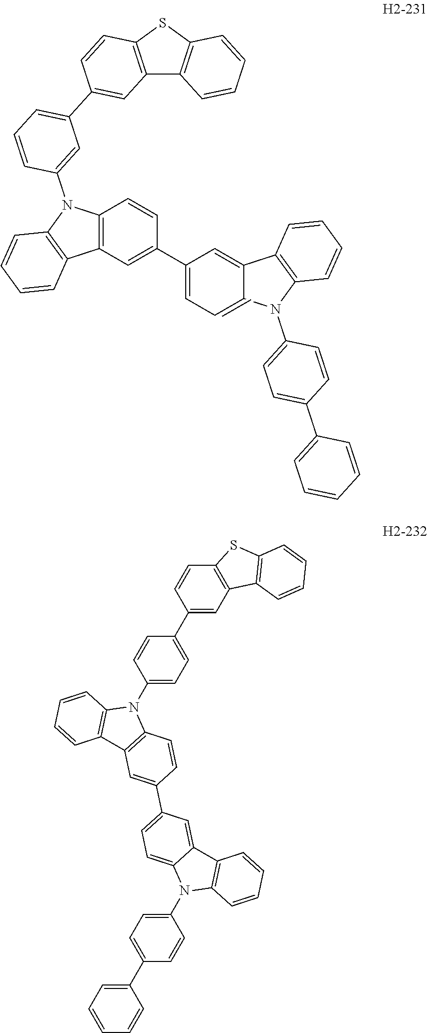

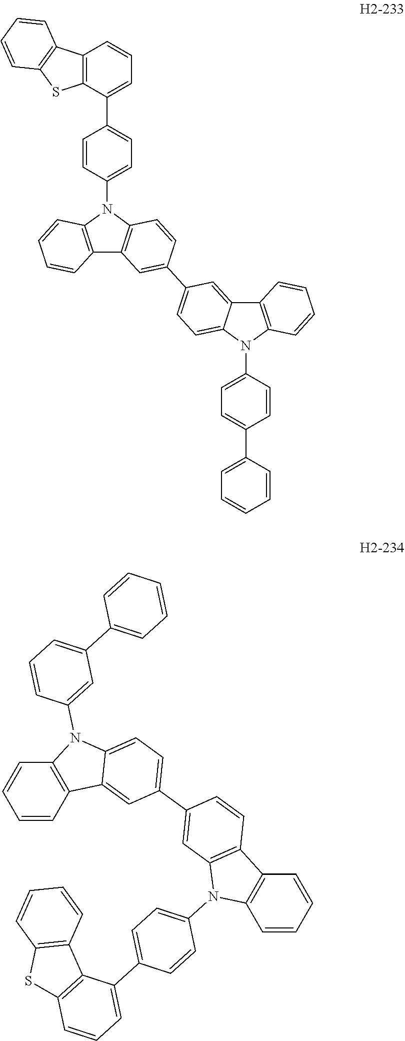 [SMILES: c1ccc(-c2ccc(-n3c4ccccc4c4cc(-c5ccc6c(c5)c5ccccc5n6-c5ccc(-c6cccc7c6sc6ccccc67)cc5)ccc43)cc2)cc1 ; c1ccc(-c2cccc(-n3c4ccccc4c4cc(-c5ccc6c7ccccc7n(-c7ccc(-c8cccc9sc%10ccccc%10c89)cc7)c6c5)ccc43)c2)cc1]

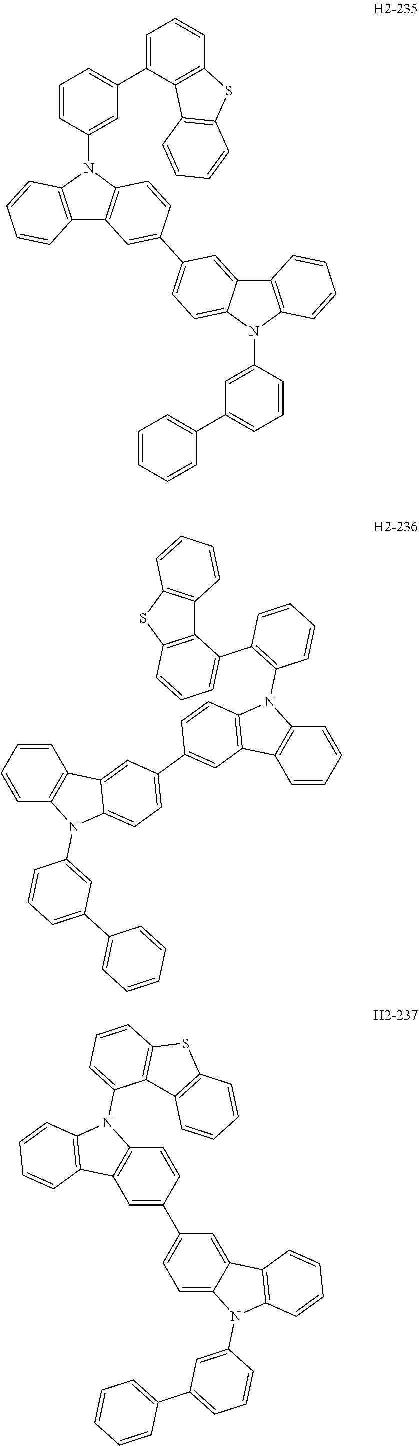

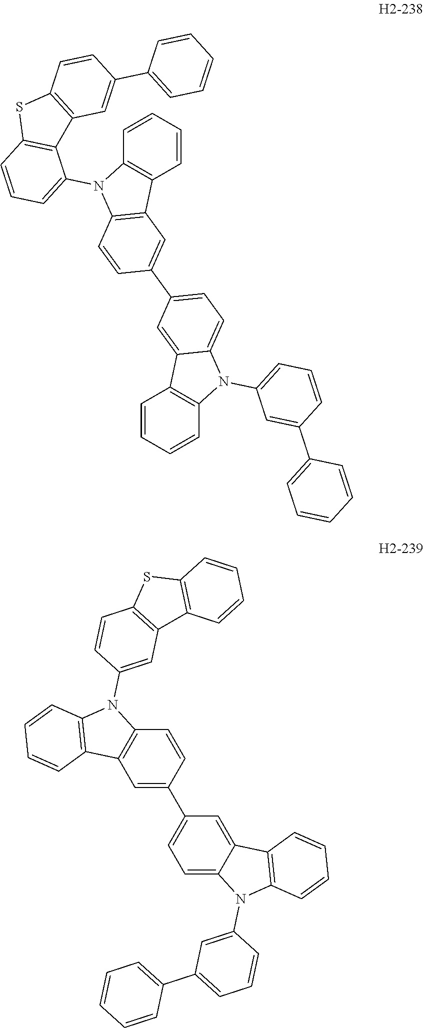 [SMILES: c1ccc(-c2cccc(-n3c4ccccc4c4cc(-c5ccc6c(c5)c5ccccc5n6-c5cccc6sc7ccc(-c8ccccc8)cc7c56)ccc43)c2)cc1 ; c1ccc(-c2cccc(-n3c4ccccc4c4cc(-c5ccc6c(c5)c5ccccc5n6-c5ccc6sc7ccccc7c6c5)ccc43)c2)cc1]

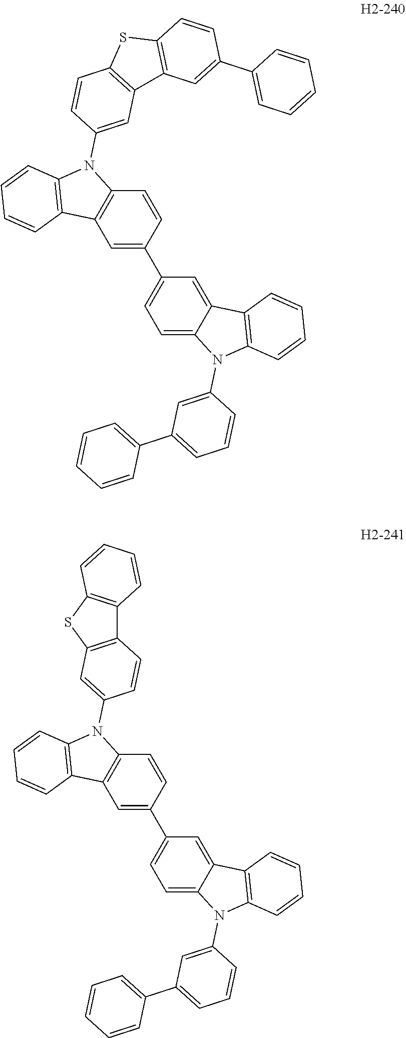 [SMILES: c1ccc(-c2cccc(-n3c4ccccc4c4cc(-c5ccc6c(c5)c5ccccc5n6-c5ccc6sc7ccc(-c8ccccc8)cc7c6c5)ccc43)c2)cc1 ; c1ccc(-c2cccc(-n3c4ccccc4c4cc(-c5ccc6c(c5)c5ccccc5n6-c5ccc6c(c5)sc5ccccc56)ccc43)c2)cc1]

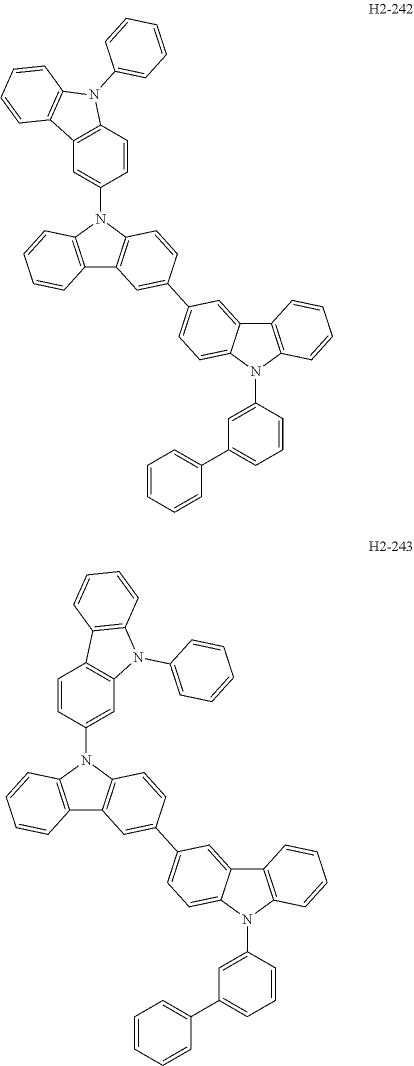 [SMILES: c1ccc(-c2cccc(-n3c4ccccc4c4cc(-c5ccc6c(c5)c5ccccc5n6-c5ccc6c(c5)c5ccccc5n6-c5ccccc5)ccc43)c2)cc1 ; c1ccc(-c2cccc(-n3c4ccccc4c4cc(-c5ccc6c(c5)c5ccccc5n6-c5ccc6c7ccccc7n(-c7ccccc7)c6c5)ccc43)c2)cc1]

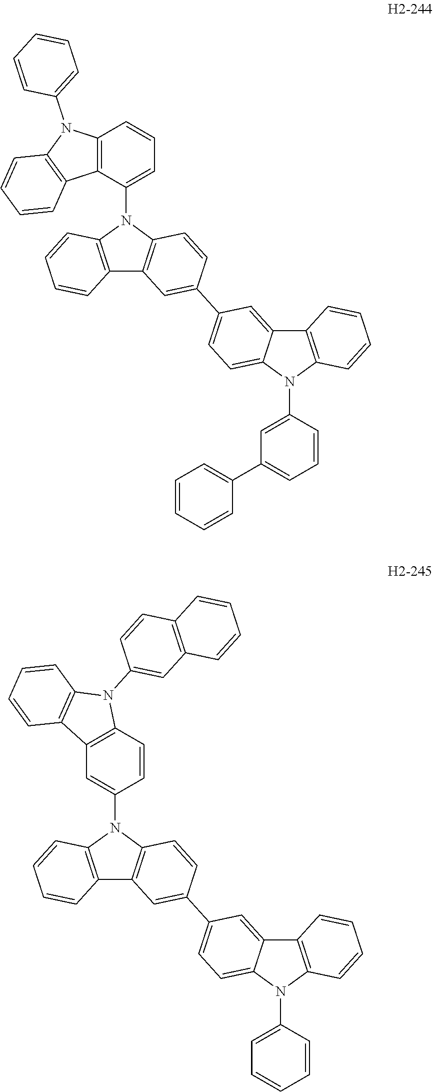 [SMILES: c1ccc(-c2cccc(-n3c4ccccc4c4cc(-c5ccc6c(c5)c5ccccc5n6-c5cccc6c5c5ccccc5n6-c5ccccc5)ccc43)c2)cc1 ; c1ccc(-n2c3ccccc3c3cc(-c4ccc5c(c4)c4ccccc4n5-c4ccc5c(c4)c4ccccc4n5-c4ccc5ccccc5c4)ccc32)cc1]

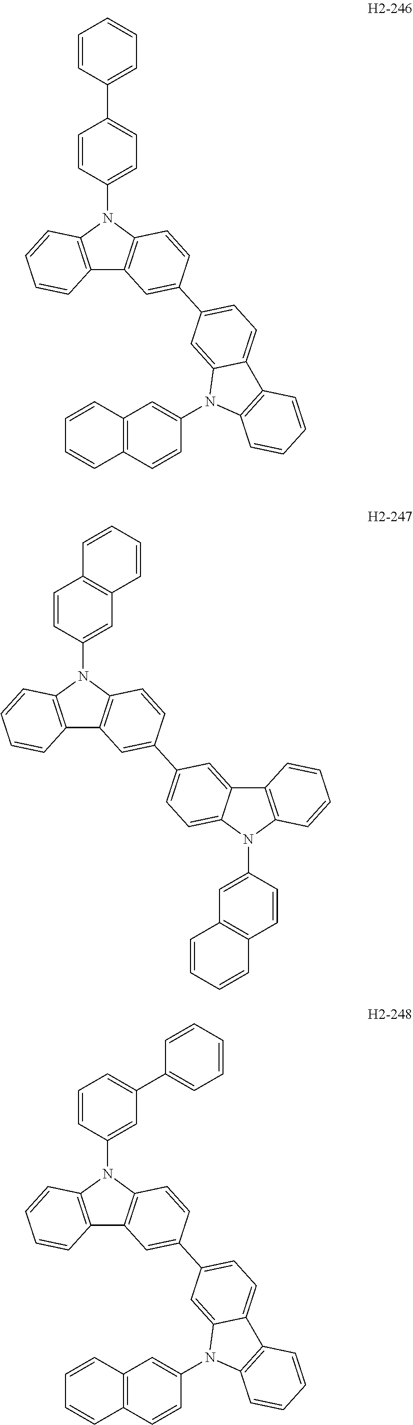

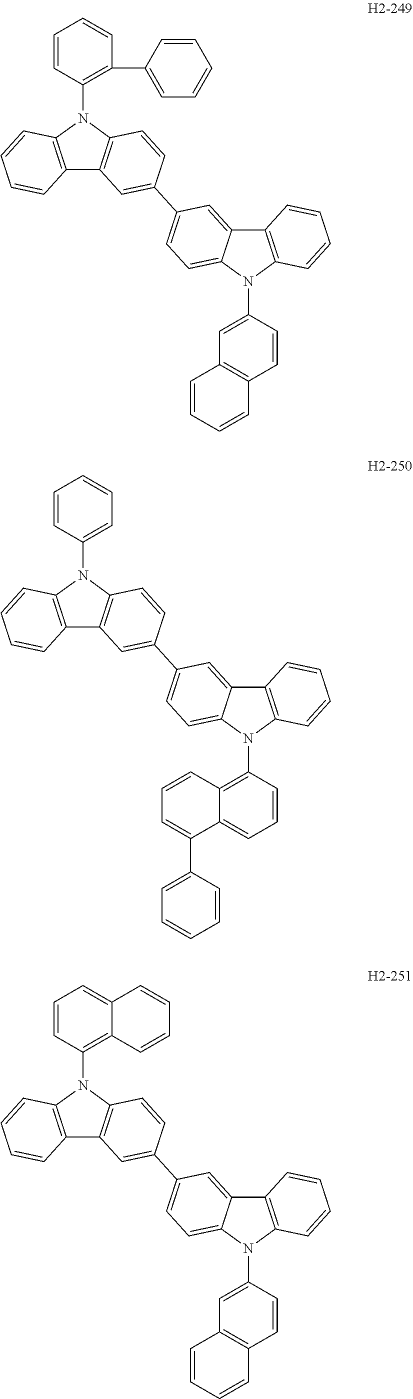 [SMILES: c1ccc(-c2ccccc2-n2c3ccccc3c3cc(-c4ccc5c(c4)c4ccccc4n5-c4ccc5ccccc5c4)ccc32)cc1 ; c1ccc(-c2cccc3c(-n4c5ccccc5c5cc(-c6ccc7c(c6)c6ccccc6n7-c6ccccc6)ccc54)cccc23)cc1 ; c1ccc2cc(-n3c4ccccc4c4cc(-c5ccc6c(c5)c5ccccc5n6-c5cccc6ccccc56)ccc43)ccc2c1]

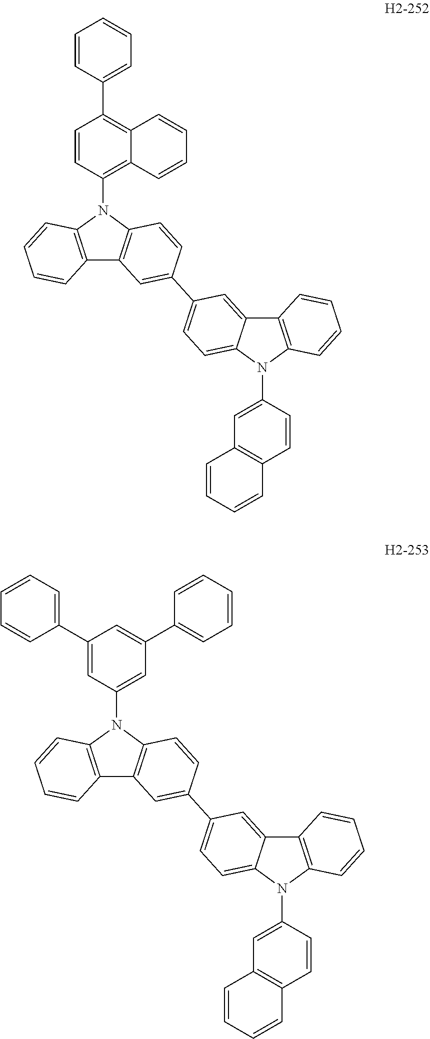 [SMILES: c1ccc(-c2ccc(-n3c4ccccc4c4cc(-c5ccc6c(c5)c5ccccc5n6-c5ccc6ccccc6c5)ccc43)c3ccccc23)cc1 ; c1ccc(-c2cc(-c3ccccc3)cc(-n3c4ccccc4c4cc(-c5ccc6c(c5)c5ccccc5n6-c5ccc6ccccc6c5)ccc43)c2)cc1]

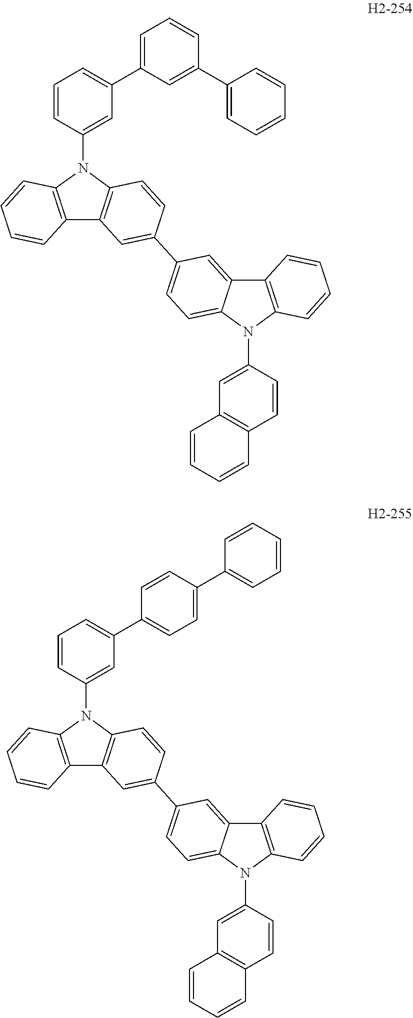 [SMILES: c1ccc(-c2cccc(-c3cccc(-n4c5ccccc5c5cc(-c6ccc7c(c6)c6ccccc6n7-c6ccc7ccccc7c6)ccc54)c3)c2)cc1 ; c1ccc(-c2ccc(-c3cccc(-n4c5ccccc5c5cc(-c6ccc7c(c6)c6ccccc6n7-c6ccc7ccccc7c6)ccc54)c3)cc2)cc1]

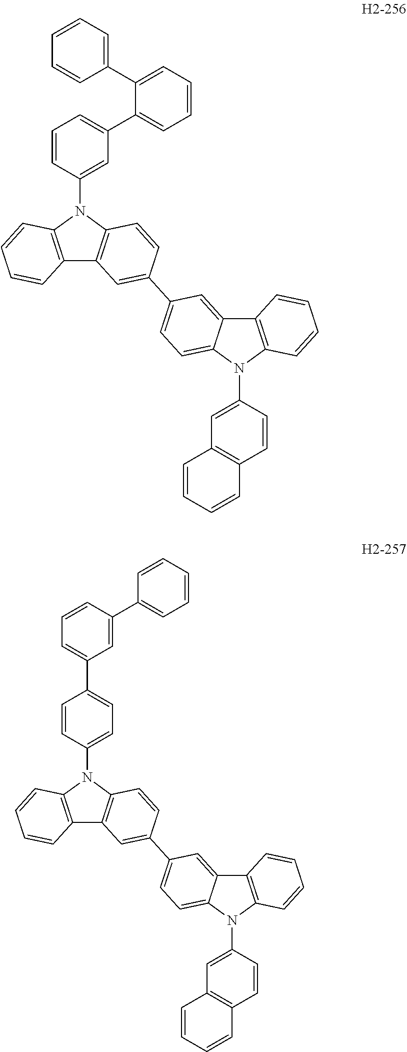 [SMILES: c1ccc(-c2ccccc2-c2cccc(-n3c4ccccc4c4cc(-c5ccc6c(c5)c5ccccc5n6-c5ccc6ccccc6c5)ccc43)c2)cc1 ; c1ccc(-c2cccc(-c3ccc(-n4c5ccccc5c5cc(-c6ccc7c(c6)c6ccccc6n7-c6ccc7ccccc7c6)ccc54)cc3)c2)cc1]

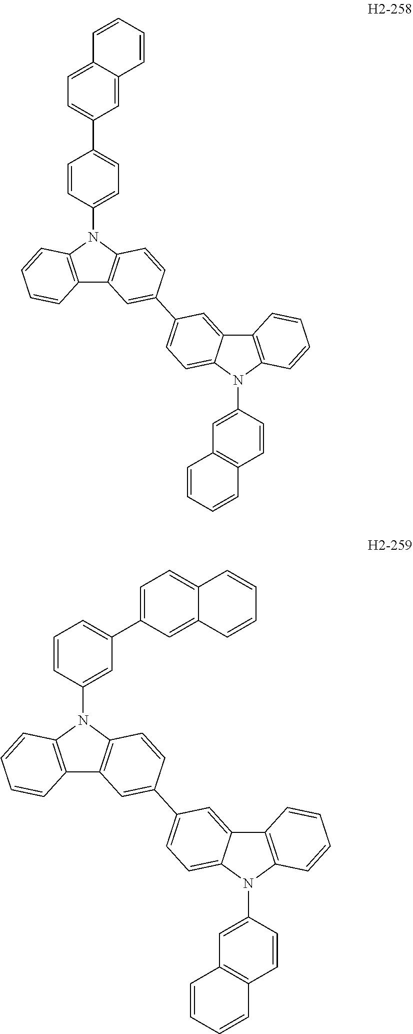 [SMILES: c1ccc2cc(-c3ccc(-n4c5ccccc5c5cc(-c6ccc7c(c6)c6ccccc6n7-c6ccc7ccccc7c6)ccc54)cc3)ccc2c1 ; c1cc(-c2ccc3ccccc3c2)cc(-n2c3ccccc3c3cc(-c4ccc5c(c4)c4ccccc4n5-c4ccc5ccccc5c4)ccc32)c1]

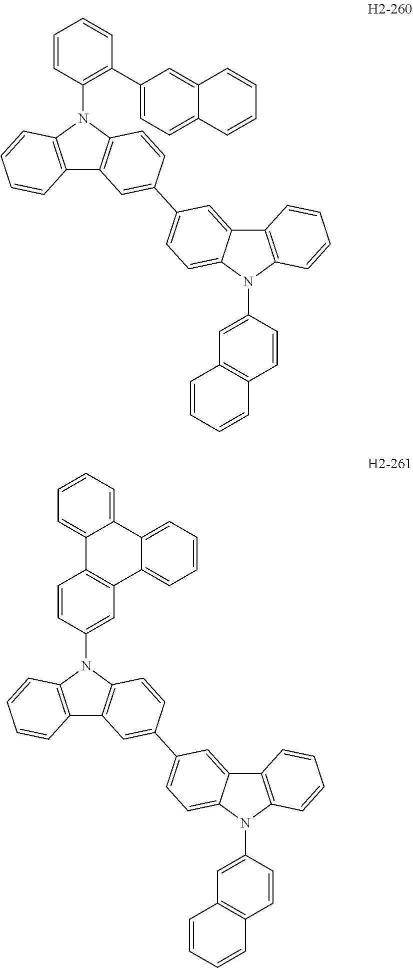 [SMILES: c1ccc(-n2c3ccccc3c3cc(-c4ccc5c(c4)c4ccccc4n5-c4ccc5ccccc5c4)ccc32)c(-c2ccc3ccccc3c2)c1 ; c1ccc2cc(-n3c4ccccc4c4cc(-c5ccc6c(c5)c5ccccc5n6-c5ccc6c7ccccc7c7ccccc7c6c5)ccc43)ccc2c1]

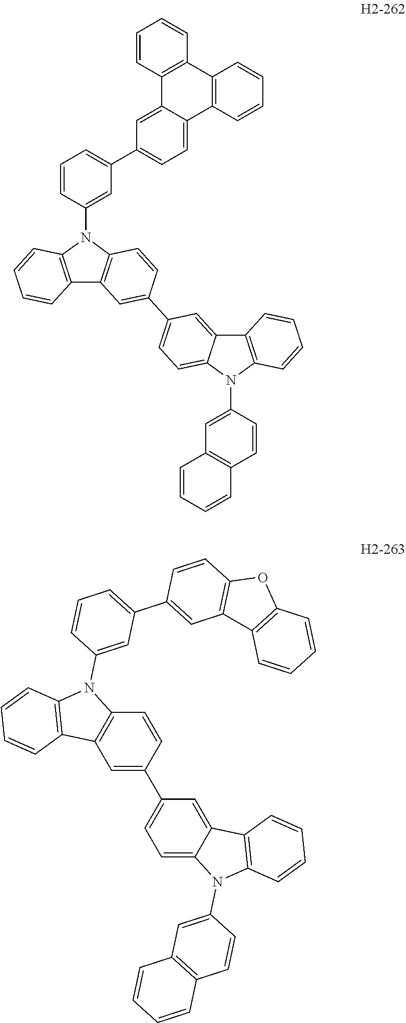 [SMILES: c1cc(-c2ccc3c4ccccc4c4ccccc4c3c2)cc(-n2c3ccccc3c3cc(-c4ccc5c(c4)c4ccccc4n5-c4ccc5ccccc5c4)ccc32)c1 ; c1cc(-c2ccc3oc4ccccc4c3c2)cc(-n2c3ccccc3c3cc(-c4ccc5c(c4)c4ccccc4n5-c4ccc5ccccc5c4)ccc32)c1]

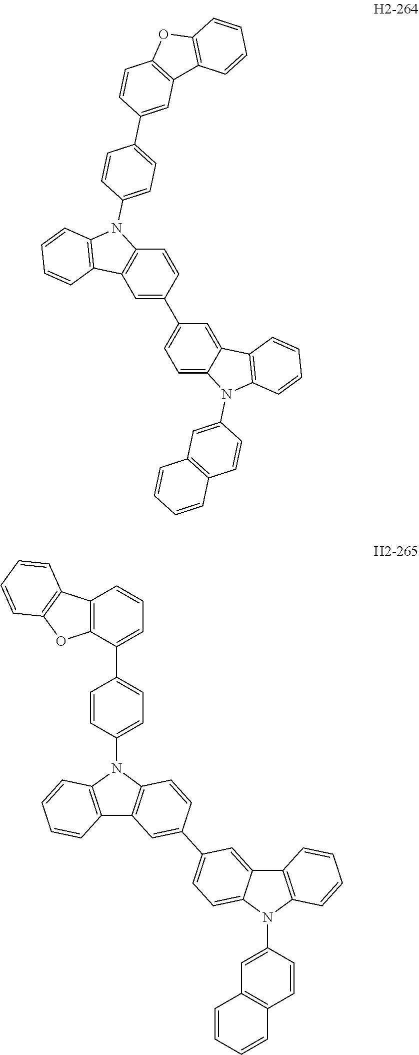 [SMILES: c1ccc2cc(-n3c4ccccc4c4cc(-c5ccc6c(c5)c5ccccc5n6-c5ccc(-c6ccc7oc8ccccc8c7c6)cc5)ccc43)ccc2c1 ; c1ccc2cc(-n3c4ccccc4c4cc(-c5ccc6c(c5)c5ccccc5n6-c5ccc(-c6cccc7c6oc6ccccc67)cc5)ccc43)ccc2c1]

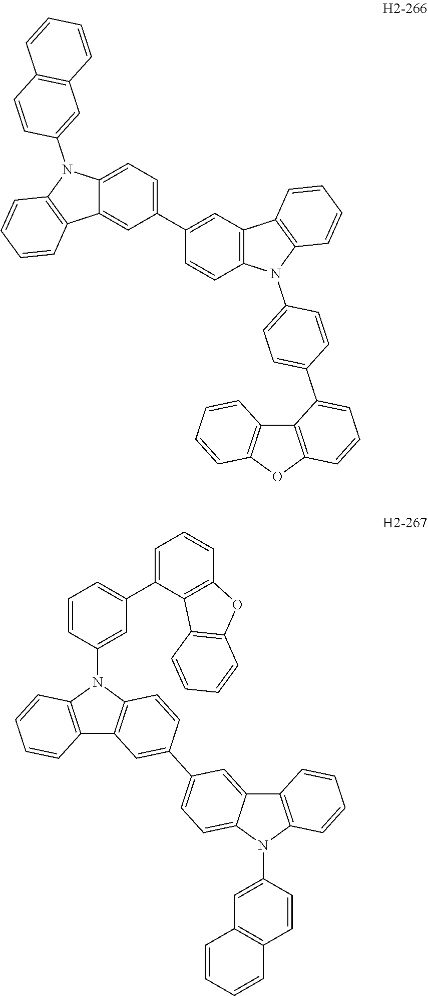 [SMILES: c1ccc2cc(-n3c4ccccc4c4cc(-c5ccc6c(c5)c5ccccc5n6-c5ccc(-c6cccc7oc8ccccc8c67)cc5)ccc43)ccc2c1 ; c1cc(-c2cccc3oc4ccccc4c23)cc(-n2c3ccccc3c3cc(-c4ccc5c(c4)c4ccccc4n5-c4ccc5ccccc5c4)ccc32)c1]

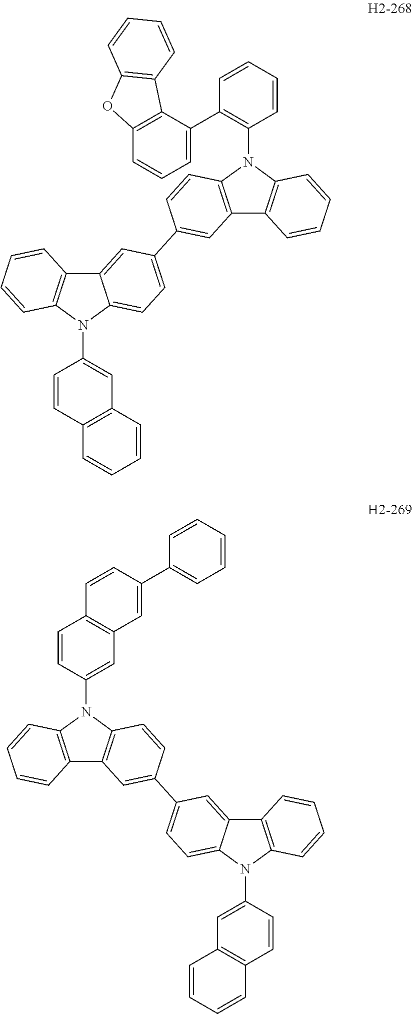 [SMILES: c1ccc(-n2c3ccccc3c3cc(-c4ccc5c(c4)c4ccccc4n5-c4ccc5ccccc5c4)ccc32)c(-c2cccc3oc4ccccc4c23)c1 ; c1ccc(-c2ccc3ccc(-n4c5ccccc5c5cc(-c6ccc7c(c6)c6ccccc6n7-c6ccc7ccccc7c6)ccc54)cc3c2)cc1]

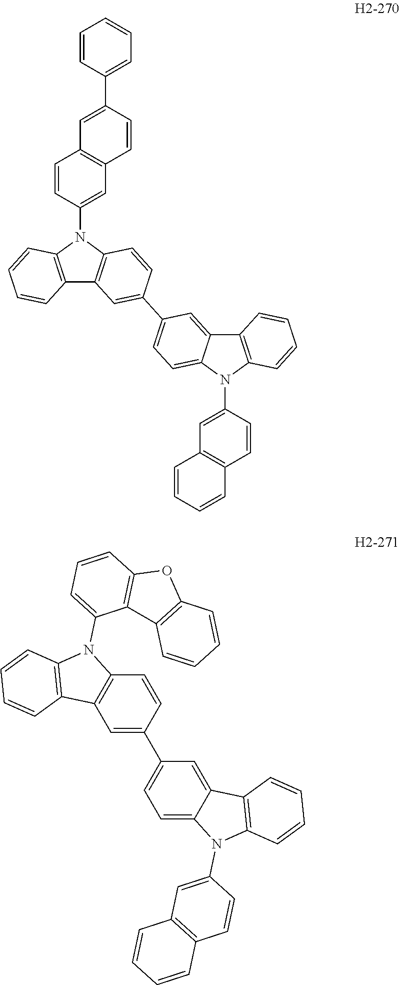 [SMILES: c1ccc(-c2ccc3cc(-n4c5ccccc5c5cc(-c6ccc7c(c6)c6ccccc6n7-c6ccc7ccccc7c6)ccc54)ccc3c2)cc1 ; c1ccc2cc(-n3c4ccccc4c4cc(-c5ccc6c(c5)c5ccccc5n6-c5cccc6oc7ccccc7c56)ccc43)ccc2c1]

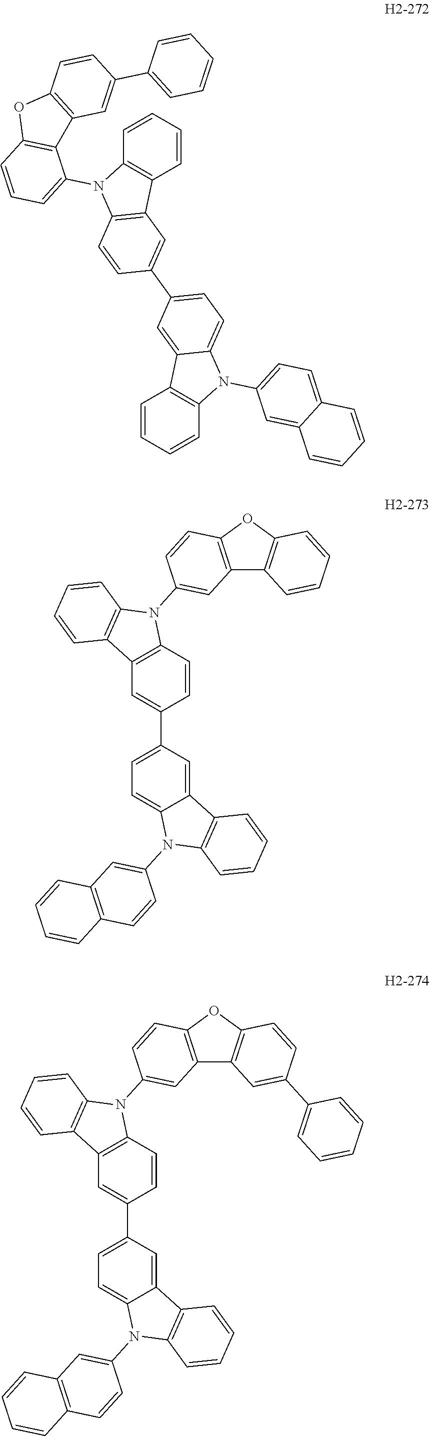 [SMILES: c1ccc(-c2ccc3oc4cccc(-n5c6ccccc6c6cc(-c7ccc8c(c7)c7ccccc7n8-c7ccc8ccccc8c7)ccc65)c4c3c2)cc1 ; c1ccc2cc(-n3c4ccccc4c4cc(-c5ccc6c(c5)c5ccccc5n6-c5ccc6oc7ccccc7c6c5)ccc43)ccc2c1 ; c1ccc(-c2ccc3oc4ccc(-n5c6ccccc6c6cc(-c7ccc8c(c7)c7ccccc7n8-c7ccc8ccccc8c7)ccc65)cc4c3c2)cc1]

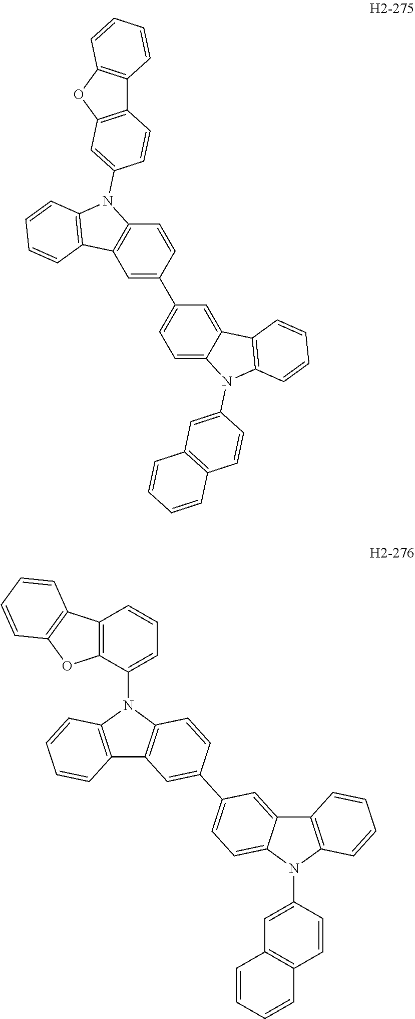 [SMILES: c1ccc2cc(-n3c4ccccc4c4cc(-c5ccc6c(c5)c5ccccc5n6-c5ccc6c(c5)oc5ccccc56)ccc43)ccc2c1 ; c1ccc2cc(-n3c4ccccc4c4cc(-c5ccc6c(c5)c5ccccc5n6-c5cccc6c5oc5ccccc56)ccc43)ccc2c1]

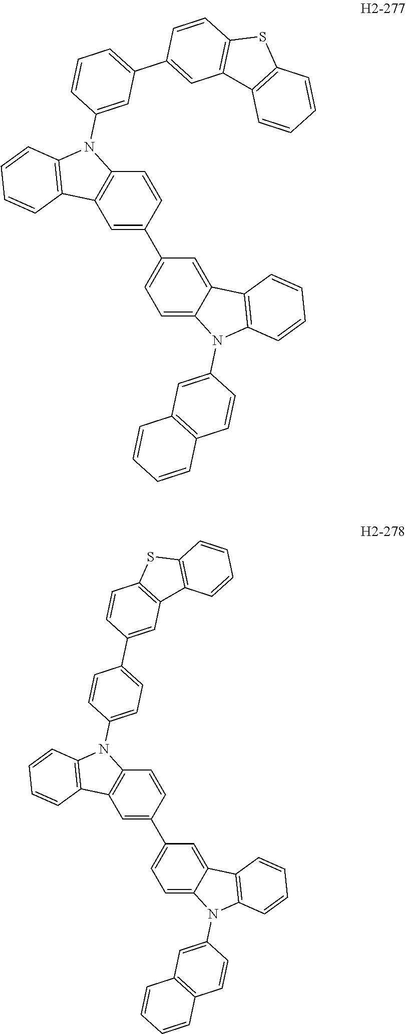 [SMILES: c1cc(-c2ccc3sc4ccccc4c3c2)cc(-n2c3ccccc3c3cc(-c4ccc5c(c4)c4ccccc4n5-c4ccc5ccccc5c4)ccc32)c1 ; c1ccc2cc(-n3c4ccccc4c4cc(-c5ccc6c(c5)c5ccccc5n6-c5ccc(-c6ccc7sc8ccccc8c7c6)cc5)ccc43)ccc2c1]

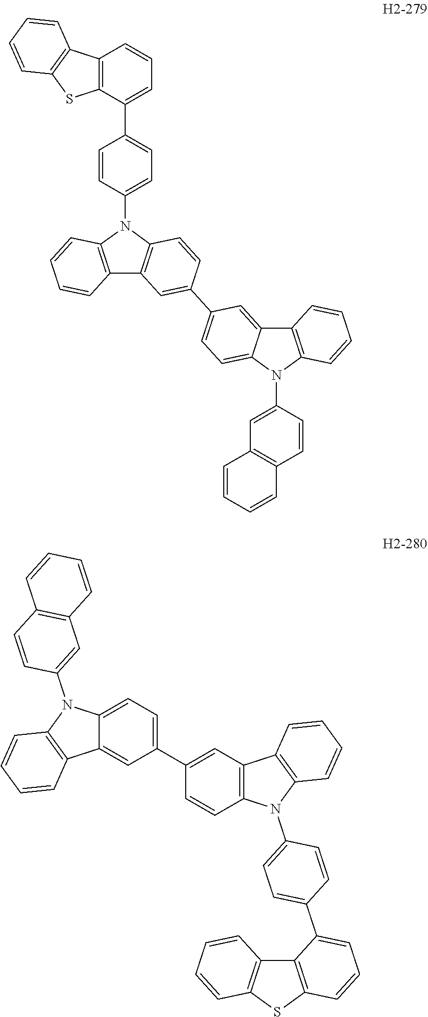 [SMILES: c1ccc2cc(-n3c4ccccc4c4cc(-c5ccc6c(c5)c5ccccc5n6-c5ccc(-c6cccc7c6sc6ccccc67)cc5)ccc43)ccc2c1 ; c1ccc2cc(-n3c4ccccc4c4cc(-c5ccc6c(c5)c5ccccc5n6-c5ccc(-c6cccc7sc8ccccc8c67)cc5)ccc43)ccc2c1]

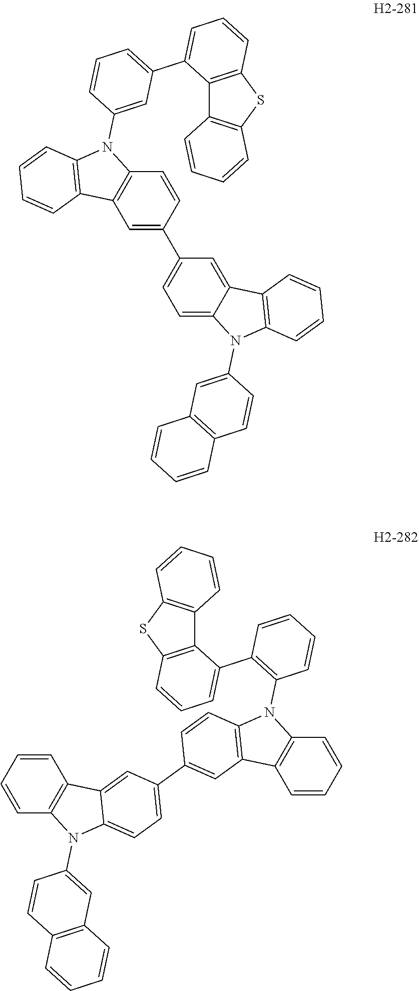 [SMILES: c1cc(-c2cccc3sc4ccccc4c23)cc(-n2c3ccccc3c3cc(-c4ccc5c(c4)c4ccccc4n5-c4ccc5ccccc5c4)ccc32)c1 ; c1ccc(-n2c3ccccc3c3cc(-c4ccc5c(c4)c4ccccc4n5-c4ccc5ccccc5c4)ccc32)c(-c2cccc3sc4ccccc4c23)c1]

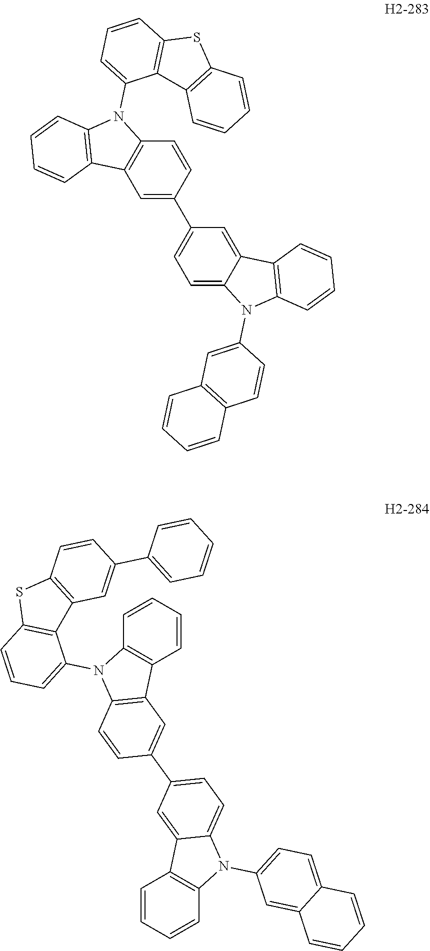 [SMILES: c1ccc2cc(-n3c4ccccc4c4cc(-c5ccc6c(c5)c5ccccc5n6-c5cccc6sc7ccccc7c56)ccc43)ccc2c1 ; c1ccc(-c2ccc3sc4cccc(-n5c6ccccc6c6cc(-c7ccc8c(c7)c7ccccc7n8-c7ccc8ccccc8c7)ccc65)c4c3c2)cc1]

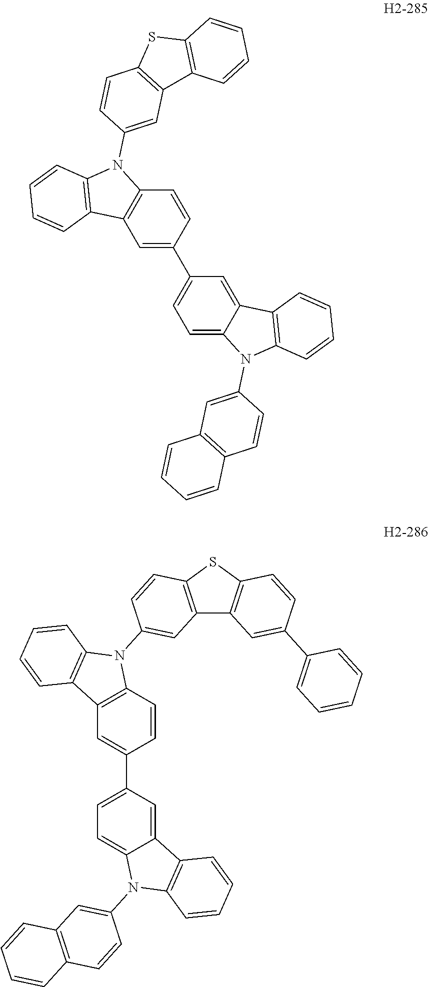 [SMILES: c1ccc2cc(-n3c4ccccc4c4cc(-c5ccc6c(c5)c5ccccc5n6-c5ccc6sc7ccccc7c6c5)ccc43)ccc2c1 ; c1ccc(-c2ccc3sc4ccc(-n5c6ccccc6c6cc(-c7ccc8c(c7)c7ccccc7n8-c7ccc8ccccc8c7)ccc65)cc4c3c2)cc1]

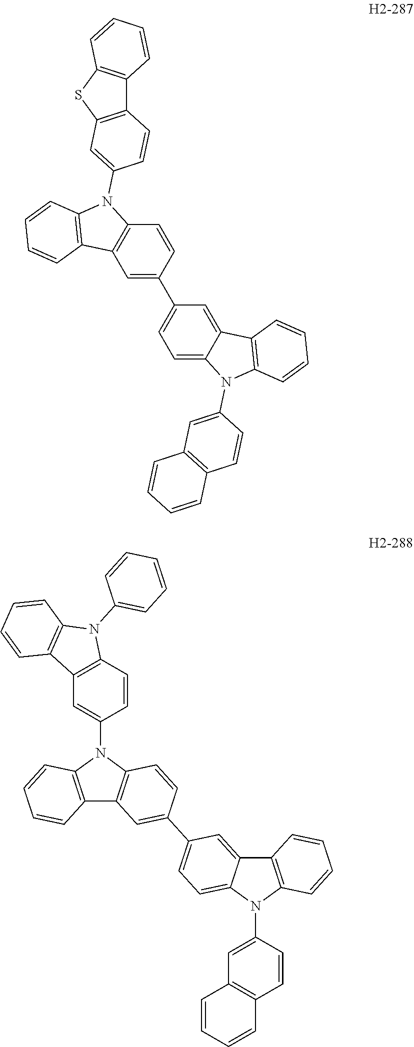 [SMILES: c1ccc2cc(-n3c4ccccc4c4cc(-c5ccc6c(c5)c5ccccc5n6-c5ccc6c(c5)sc5ccccc56)ccc43)ccc2c1 ; c1ccc(-n2c3ccccc3c3cc(-n4c5ccccc5c5cc(-c6ccc7c(c6)c6ccccc6n7-c6ccc7ccccc7c6)ccc54)ccc32)cc1]

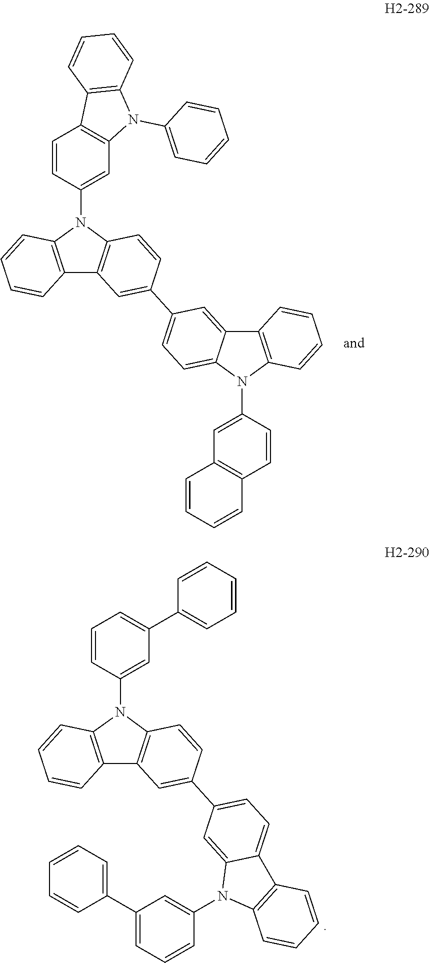

20. An organic electroluminescent device comprising a first electrode; a second electrode; and at least one light-emitting layer between the first electrode and the second electrode, and wherein at least one layer of the light-emitting layer contains a plurality of host materials according to claim 1.

21. An organic electroluminescent device comprising a first electrode; a second electrode; and at least one light-emitting layer between the first electrode and the second electrode, and wherein at least one layer of the light-emitting layer contains an organic electroluminescent compound according to claim 15.

22. An organic electroluminescent device comprising a first electrode; a second electrode; and at least one light-emitting layer between the first electrode and the second electrode, and wherein at least one layer of the light-emitting layer contains a plurality of host materials according to claim 18.

23. A plurality of host materials comprising three or more host materials comprising at least one first host compound and at least one second host compound, wherein the structural backbone of the first host compounds and the second host compounds is the same, but the number of hydrogen and deuterium atoms in each compound is different.

24. The plurality of host materials according to claim 23, wherein the first host compound does not contain deuterium and the second host compound contains deuterium.

25. The plurality of host materials according to claim 24, wherein the structural backbone of the first host compound and the second host compound is represented by Formula 2. 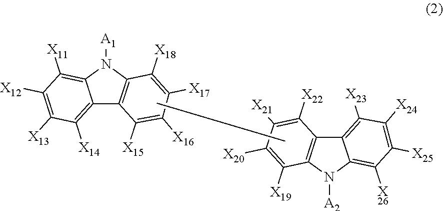
wherein,
A₁ and A₂ each independently represent a substituted or unsubstituted (C6-C30)aryl, a substituted or unsubstituted dibenzofuranyl, a substituted or unsubstituted dibenzothiophenyl, or a substituted or unsubstituted carbazoly;
any one of X₁₅ to X₁₈ and any one of X₁₉ to X₂₂ are connected to each other to form a single bond; and
X₁₁ to X₁₄, X₂₃ to X₂₆, and X₁₅ to X₂₂, which do not form a single bond, each independently represent hydrogen, deuterium, a substituted or unsubstituted (C6-C30)aryl, or a substituted or unsubstituted (3- to 30-membered)heteroaryl; or may be linked to an adjacent substituent to form a ring(s).

26. The plurality of host materials according to claim 23, wherein the plurality of host materials further comprises a third host compound, wherein the third host compound is represented by Formula 1-11: 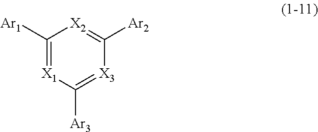
wherein,
X₁ to X₃ each independently represent N or CRₐ; provided that at least two of X₁ to X₃ are N;
Rₐ represents hydrogen or deuterium; and
Ar₁ to Ar₃ each independently represent, a substituted or unsubstituted (C6-C30)aryl or a substituted or unsubstituted (3- to 30-membered)heteroaryl.
